(19) United States
(12) Patent Application Publication
(10) Pub. No.: US 2026/0053785 A1
(43) Pub. Date: Feb. 26, 2026

(54) COMBINATION THERAPY FOR TREATMENT OF CANCER

(71) Applicant: PMV Pharmaceuticals, Inc., Princeton, NJ (US)

(72) Inventors: Melissa DUMBLE, Watchung, NJ (US); Anna PUZIO-KUTER, Hillsborough, NJ (US); Binh VU, North Caldwell, NJ (US); Tamer AHMED, Edison, NJ (US)

(21) Appl. No.: 19/373,216

(22) Filed: Oct. 29, 2025

Related U.S. Application Data
(63) Continuation of application No. PCT/US24/28982, filed on May 10, 2024.

(51) Int. Cl. A61K 31/454; A61K 31/4439; A61K 31/496; A61P 35/00

(52) U.S. Cl. CPC A61K 31/454; A61K 31/4439; A61K 31/496; A61P 35/00

(57) ABSTRACT

Mutations in oncogenes and tumor suppressors contribute to the development and progression of cancer. The present disclosure describes methods of recovering wild-type function to p53 mutants by treating a tumor with a compound and a second agent. The compounds of the present invention can bind to mutant p53 and restore the ability of the p53 mutant to bind DNA and activate downstream effectors involved in tumor suppression. The disclosed compounds can be used in combination with an MDM2, PI3K, or AKT inhibitor to reduce the progression of cancers that contain a p53 mutation.

### CROSS-REFERENCE TO RELATED APPLICATIONS

This application is a continuation of International Application No. PCT/US2024/028982, filed May 10, 2024, which claims the benefit of U.S. Provisional Application No. 63/501,924, filed May 12, 2023, each of which is incorporated herein by reference in its entirety.

### BACKGROUND

Cancer, an uncontrolled proliferation of cells, is a multifactorial disease characterized by tumor formation, growth, and in some instances, metastasis. Cells carrying an activated oncogene, damaged genome, or other cancer-promoting alterations can be prevented from replicating through an elaborate tumor suppression network. A central component of this tumor suppression network is p53, one of the most potent tumor suppressors in the cell. Both the wild type and mutant conformations of p53 are implicated in the progression of cancer.

### INCORPORATION BY REFERENCE

Each patent, publication, and non-patent literature cited in the application is hereby incorporated by reference in its entirety as if each was incorporated by reference individually.

### SUMMARY OF THE INVENTION

Provided herein is a method of treating a solid tumor in a subject in need thereof, the method comprising: (i) administering to the subject a therapeutically-effective amount of a compound, wherein the compound binds to a mutant p53 protein and reconforms the mutant p53 protein to a conformation of p53 that exhibits anti-tumor activity; and (ii) administering to the subject a therapeutically-effective amount of an MDM2 inhibitor.

Also provided herein is a method of treating a solid tumor in a subject in need thereof, the method comprising: (i) administering to the subject a therapeutically-effective amount of a compound that increases anti-cancer activity of a mutant p53 protein in the subject; and (ii) administering to the subject a therapeutically-effective amount of an MDM2 inhibitor.

Further provided herein is a method of treating a solid tumor, the method comprising: (i) administering to a subject in need thereof a therapeutically-effective amount of a compound that binds to a mutant p53 protein and reconforms the mutant p53 protein to a conformation of p53 that exhibits anti-cancer activity; and (ii) administering to the subject a therapeutically-effective amount of an MDM2 inhibitor, wherein if in a controlled study of treatment of the cancer in a first patient population and a second patient population: (a) a first median survival time of the first patient population is determined, wherein the first patient population is treated with the therapeutically-effective amount of the compound that binds to a mutant p53 protein and reconforms the mutant p53 protein to a conformation of p53 that exhibits anti-cancer activity; and (b) a second median survival time of the second patient population is determined, wherein the second patient population is treated with the therapeutically-effective amount of the compound that binds to a mutant p53 protein and reconforms the mutant p53 protein to a conformation of p53 that exhibits anti-tumor activity and the therapeutically-effective amount of the MDM2 inhibitor; then the second median survival time is at least about 50% greater than is the first median survival time.

Provided herein is a method of treating a solid tumor in a subject in need thereof, the method comprising: (i) administering to the subject a therapeutically-effective amount of a compound, wherein the compound binds to a mutant p53 protein and reconforms the mutant p53 protein to a conformation of p53 that exhibits anti-tumor activity; and (ii) administering to the subject a therapeutically-effective amount of a PI3K inhibitor.

Also provided herein is a method of treating a solid tumor in a subject in need thereof, the method comprising: (i) administering to the subject a therapeutically-effective amount of a compound that increases anti-cancer activity of a mutant p53 protein in the subject; and (ii) administering to the subject a therapeutically-effective amount of a PI3K inhibitor.

Further provided herein is a method of treating a solid tumor, the method comprising: (i) administering to a subject in need thereof a therapeutically-effective amount of a compound that binds to a mutant p53 protein and reconforms the mutant p53 protein to a conformation of p53 that exhibits anti-cancer activity; and (ii) administering to the subject a therapeutically-effective amount of a PI3K inhibitor, wherein if in a controlled study of treatment of the cancer in a first patient population and a second patient population: (a) a first median survival time of the first patient population is determined, wherein the first patient population is treated with the therapeutically-effective amount of the compound that binds to a mutant p53 protein and reconforms the mutant p53 protein to a conformation of p53 that exhibits anti-cancer activity; and (b) a second median survival time of the second patient population is determined, wherein the second patient population is treated with the therapeutically-effective amount of the compound that binds to a mutant p53 protein and reconforms the mutant p53 protein to a conformation of p53 that exhibits anti-tumor activity and the therapeutically-effective amount of the PI3K inhibitor; then the second median survival time is at least about 50% greater than is the first median survival time.

Provided herein is a method of treating a solid tumor in a subject in need thereof, the method comprising: (i) administering to the subject a therapeutically-effective amount of a compound, wherein the compound binds to a mutant p53 protein and reconforms the mutant p53 protein to a conformation of p53 that exhibits anti-tumor activity; and (ii) administering to the subject a therapeutically-effective amount of an AKT inhibitor.

Also provided herein is a method of treating a solid tumor in a subject in need thereof, the method comprising: (i) administering to the subject a therapeutically-effective amount of a compound that increases anti-cancer activity of a mutant p53 protein in the subject; and (ii) administering to the subject a therapeutically-effective amount of an AKT inhibitor.

Further provided herein is a method of treating a solid tumor, the method comprising: (i) administering to a subject in need thereof a therapeutically-effective amount of a compound that binds to a mutant p53 protein and reconforms the mutant p53 protein to a conformation of p53 that exhibits anti-cancer activity; and (ii) administering to the subject a therapeutically-effective amount of an AKT inhibitor, wherein if in a controlled study of treatment of the cancer in a first patient population and a second patient population: (a) a first median survival time of the first patient population is determined, wherein the first patient population is treated with the therapeutically-effective amount of the compound that binds to a mutant p53 protein and reconforms the mutant p53 protein to a conformation of p53 that exhibits anti-cancer activity; and (b) a second median survival time of the second patient population is determined, wherein the second patient population is treated with the therapeutically-effective amount of the compound that binds to a mutant p53 protein and reconforms the mutant p53 protein to a conformation of p53 that exhibits anti-tumor activity and the therapeutically-effective amount of the AKT inhibitor; then the second median survival time is at least about 50% greater than is the first median survival time.

### BRIEF DESCRIPTION OF THE FIGURES

FIG. 1 shows changes in % survival in a mouse xenograft model of gastric cancer (NUGC3) over 90 days upon receiving treatment with vehicle (QD); Compound 1, 50 mg/kg (QD); MDM2 inhibitor RG7388 20 mg/kg (QD); Compound 1, 100 mg/kg (QD); Compound 1, 50 mg/kg (QD) simultaneously with MDM2 inhibitor RG7388, 20 mg/kg (QD); or Compound 1, 100 mg/kg (QD) simultaneously with MDM2 inhibitor RG7388, 20 mg/kg (QD).

FIG. 2 shows changes in tumor volume (mm³) in a mouse xenograft model of gastric cancer (NUGC3-71) over 15 days upon receiving treatment with vehicle (QD); MDM2 inhibitor AMG232 20 mg/kg (QD); Compound 1, 50 mg/kg (QD); Compound 1, 100 mg/kg (QD); Compound 1, 50 mg/kg (QD)+MDM2 inhibitor AMG232 20 mg/kg (QD); or Compound 1, 100 mg/kg (QD)+MDM2 inhibitor AMG232 20 mg/kg (QD); Compound 1, 50 mg/kg (QD) 6 hours apart from treatment with MDM2 inhibitor AMG232, 20 mg/kg (QD); or Compound 1, 100 mg/kg (QD) 6 hours apart from treatment with MDM2 inhibitor AMG232, 20 mg/kg (QD).

FIG. 3A shows changes in tumor volume (mm³) in a mouse xenograft model of gastric cancer (NUGC3-71) over 15 days upon receiving treatment with vehicle (QD); MDM2 inhibitor RAIN-32 (milademetan) 20 mg/kg (QD); Compound 1, 50 mg/kg (QD); Compound 1, 100 mg/kg (QD); Compound 1, 50 mg/kg (QD)+MDM2 inhibitor RAIN-32 20 mg/kg (QD); or Compound 1, 100 mg/kg (QD)+MDM2 inhibitor RAIN-32 20 mg/kg (QD); Compound 1, 50 mg/kg (QD) 6 hours apart from treatment with MDM2 inhibitor RAIN-32, 20 mg/kg (QD); or Compound 1, 100 mg/kg (QD) 6 hours apart from treatment with MDM2 inhibitor RAIN-32, 20 mg/kg (QD).

FIG. 3B shows the changes in percent body weight change in mice implanted with NUGC3-71 mouse syngeneic gastric cancer tumors upon receiving treatment with vehicle (QD); MDM2 inhibitor RAIN-32 20 mg/kg (QD); Compound 1, 50 mg/kg (QD); Compound 1, 100 mg/kg (QD); Compound 1, 50 mg/kg (QD)+MDM2 inhibitor RAIN-32 20 mg/kg (QD); or Compound 1, 100 mg/kg (QD)+MDM2 inhibitor RAIN-32 20 mg/kg (QD); Compound 1, 50 mg/kg (QD) 6 hours apart from treatment with MDM2 inhibitor RAIN-32, 20 mg/kg (QD); or Compound 1, 100 mg/kg (QD) 6 hours apart from treatment with MDM2 inhibitor RAIN-32, 20 mg/kg (QD).

FIG. 4A shows the changes in tumor volume (mm³) in eight individual mice implanted with NUGC3-70 cancer tumors upon receiving treatment with Compound 1, 100 mg/kg (QD).

FIG. 4B shows the changes in tumor volume (mm³) in eight individual mice implanted with NUGC3-70 cancer tumors upon receiving treatment with Compound 1, 100 mg/kg (QD) dosed simultaneous with MDM2 inhibitor AMG232 20 mg/kg (QD).

FIG. 4C shows the changes in tumor volume (mm³) in eight individual mice implanted with NUGC3-70 cancer tumors upon receiving treatment with Compound 1, 100 mg/kg (QD) dosed 6 hours apart from treatment with MDM2 inhibitor AMG232, 20 mg/kg (QD).

FIG. 5A shows changes in tumor volume (mm³) in a mouse xenograft model of gastric cancer (NUGC3-71) over 80 days upon receiving treatment with vehicle (QD); MDM2 inhibitor AMG232 20 mg/kg (QD); Compound 1, 50 mg/kg (QD); Compound 1, 100 mg/kg (QD); Compound 1, 50 mg/kg (QD)+MDM2 inhibitor AMG232 20 mg/kg (QD); or Compound 1, 100 mg/kg (QD)+MDM2 inhibitor AMG232 20 mg/kg (QD); Compound 1, 50 mg/kg (QD) 6 hours apart from treatment with MDM2 inhibitor AMG232, 20 mg/kg (QD); or Compound 1, 100 mg/kg (QD) 6 hours apart from treatment with MDM2 inhibitor AMG232, 20 mg/kg (QD).

FIG. 5B shows changes in % survival in a mouse xenograft model of gastric cancer (NUGC3-70) over 80 days upon receiving treatment with vehicle (QD); MDM2 inhibitor AMG232 20 mg/kg (QD); Compound 1, 50 mg/kg (QD); Compound 1, 100 mg/kg (QD); Compound 1, 50 mg/kg (QD)+MDM2 inhibitor AMG232 20 mg/kg (QD); or Compound 1, 100 mg/kg (QD)+MDM2 inhibitor AMG232 20 mg/kg (QD); Compound 1, 50 mg/kg (QD) 6 hours apart from treatment with MDM2 inhibitor AMG232, 20 mg/kg (QD); or Compound 1, 100 mg/kg (QD) 6 hours apart from treatment with MDM2 inhibitor AMG232, 20 mg/kg (QD).

FIG. 5C shows the changes in percent body weight change in mice implanted with NUGC3-71 mouse syngeneic gastric cancer tumors upon receiving treatment with vehicle (QD); MDM2 inhibitor AMG232 20 mg/kg (QD); Compound 1, 50 mg/kg (QD); Compound 1, 100 mg/kg (QD); Compound 1, 50 mg/kg (QD)+MDM2 inhibitor AMG232 20 mg/kg (QD); or Compound 1, 100 mg/kg (QD)+MDM2 inhibitor AMG232 20 mg/kg (QD); Compound 1, 50 mg/kg (QD) 6 hours apart from treatment with MDM2 inhibitor AMG232, 20 mg/kg (QD); or Compound 1, 100 mg/kg (QD) 6 hours apart from treatment with MDM2 inhibitor AMG232, 20 mg/kg (QD).

FIG. 6A shows changes in percent viability (mm³) in an MTT cell model of gastric cancer (NUGC3) upon receiving treatment with vehicle; MDM2 inhibitor nutlin-3a 0-10 mM together with Compound 1 0.01-10 mM.

FIG. 6B shows changes in percent viability (mm³) in an MTT cell model of gastric cancer (NUGC3) upon receiving treatment with vehicle; MDM2 inhibitor RG7112 0-1 mM together with Compound 1 0-10 mM.

FIG. 6C shows changes in percent viability (mm³) in an MTT cell model of gastric cancer (NUGC3) upon receiving treatment with vehicle; MDM2 inhibitor RG7338 0-10 mM together with Compound 1 0.01-10 mM.

FIG. 7A shows changes in percent viability (mm³) in an MTT cell model of pancreatic cancer (T3M4) upon receiving treatment with vehicle; MDM2 inhibitor nutlin-3a 0-10 mM together with Compound 1 0.01-10 mM.

FIG. 7B shows changes in percent viability (mm³) in an MTT cell model of pancreatic cancer (T3M4) upon receiving treatment with vehicle; MDM2 inhibitor RG7112 0-1 mM together with Compound 1 0-10 mM.

FIG. 7C shows changes in percent viability (mm³) in an MTT cell model of pancreatic cancer (T3M4) upon receiving treatment with vehicle; MDM2 inhibitor RG7338 0-10 mM together with Compound 1 0.01-10 mM.

FIGS. 8A-8L show highest single agent (HSA) synergy scores between PI3K inhibitor alpelisib (0.078-10 mM) and Compound 1 (0.039-10 mM) as calculated by Combenefit software in p53 Y220C cell models of cancer.

FIGS. 9A-9B show changes in percent viability (FIG. 9A) and Loewe synergy scores as calculated by Combenefit software (FIG. 9B) in a CTG cell model of lung cancer (HCC2935) treated with vehicle, or PI3K inhibitor alpelisib (0.078-10 mM) together with Compound 1 (0.039-10 mM).

FIGS. 9C-9D show changes in percent viability (FIG. 9C) and Loewe synergy scores as calculated by Combenefit software (FIG. 9D) in a CTG cell model of lung cancer (HCC2935) treated with vehicle, or PI3K inhibitor inavolisib (0.078-10 mM) together with Compound 1 (0.039-10 mM).

FIGS. 9E-9F show changes in percent viability (FIG. 9E) and Loewe synergy scores as calculated by Combenefit software (FIG. 9F) in a CTG cell model of lung cancer (HCC2935) treated with vehicle, or PI3K inhibitor serabelisib (0.07810 mM) together with Compound 1 (0.039-10 mM).

FIGS. 9G-9H show changes in percent viability (FIG. 9G) and Loewe synergy scores as calculated by Combenefit software (FIG. 9H) in a CTG cell model of lung cancer (HCC2935) treated with vehicle, or PI3K inhibitor GSK2636771 (GSK263) (0.078-10 mM) together with Compound 1 (0.039-10 mM).

FIGS. 9I-9J show changes in percent viability (FIG. 9I) and Loewe synergy scores as calculated by Combenefit software (FIG. 9J) in a CTG cell model of lung cancer (HCC2935) treated with vehicle, or PI3K inhibitor eganelisib (0.078-10 mM) together with Compound 1 (0.039-10 mM).

FIGS. 9K-9L show changes in percent viability (FIG. 9K) and Loewe synergy scores as calculated by Combenefit software (FIG. 9L) in a CTG cell model of lung cancer (HCC2935) treated with vehicle, or PI3K inhibitor idelalisib (0.078-10 mM) together with Compound 1 (0.039-10 mM).

FIGS. 9M-9N show changes in percent viability (FIG. 9M) and Loewe synergy scores as calculated by Combenefit software (FIG. 9N) in a CTG cell model of lung cancer (HCC2935) treated with vehicle, or PI3K inhibitor duvelisib (0.078-10 mM) together with Compound 1 (0.039-10 mM).

FIGS. 9O-9P show changes in percent viability (FIG. 9O) and Loewe synergy scores as calculated by Combenefit software (FIG. 9P) in a CTG cell model of lung cancer (HCC2935) treated with vehicle, or AKT inhibitor MK2206 (0.078-10 mM) together with Compound 1 (0.039-10 mM).

FIGS. 9Q-9R show changes in percent viability (FIG. 9Q) and Loewe synergy scores as calculated by Combenefit software (FIG. 9R) in a CTG cell model of lung cancer (HCC2935) treated with vehicle, or AKT inhibitor ipataserib (0.078-10 mM) together with Compound 1 (0.039-10 mM).

FIGS. 9S-9T show changes in percent viability (FIG. 9S) and Loewe synergy scores as calculated by Combenefit software (FIG. 9T) in a CTG cell model of lung cancer (HCC2935) treated with vehicle, or AKT inhibitor capivasertib (0.03-10 mM) together with Compound 1 (0.039-10 mM).

FIGS. 9U-AC show percent viability in HCC2935 cells treated with Compound 1 (0.04-10 mM) in combination with alpelisib (FIG. 9U), inavolisib (FIG. 9V), serabelisib (FIG. 9W), GSK2636771 (GSK263) (FIG. 9X), eganelisib (FIG. 9Y), idelalisib (FIG. 9Z), duvelisib (FIG. 9AA), MK2206 (FIG. 9AB), and ipatasertib (FIG. 9AC).

FIGS. 10A-10B show changes in percent viability (FIG. 10A) and Loewe synergy scores as calculated by Combenefit software (FIG. 10B) in a CTG cell model of gastric cancer (SNUNCC19) treated with vehicle, or PI3K inhibitor alpelisib (0.078-10 mM) together with Compound 1 (0.039-10 mM).

FIGS. 10C-10D show changes in percent viability (FIG. 10C) and Loewe synergy scores as calculated by Combenefit software (FIG. 10D) in a CTG cell model of gastric cancer (SNUNCC19) treated with vehicle, or PI3K inhibitor inavolisib (0.078-10 mM) together with Compound 1 (0.039-10 mM).

FIGS. 10E-10F show changes in percent viability (FIG. 10E) and Loewe synergy scores as calculated by Combenefit software (FIG. 10F) in a CTG cell model of gastric cancer (SNUNCC19) treated with vehicle, or PI3K inhibitor serabelisib (0.078-10 mM) together with Compound 1 (0.039-10 mM).

FIGS. 10G-10H show changes in percent viability (FIG. 10G) and Loewe synergy scores as calculated by Combenefit software (FIG. 10H) in a CTG cell model of gastric cancer (SNUNCC19) treated with vehicle, or PI3K inhibitor GSK2636771 (GSK263) (0.078-10 mM) together with Compound 1 (0.039-10 mM).

FIGS. 10I-10J show changes in percent viability (FIG. 10I) and Loewe synergy scores as calculated by Combenefit software (FIG. 10J) in a CTG cell model of gastric cancer (SNUNCC19) treated with vehicle, or PI3K inhibitor eganelisib (0.078-10 mM) together with Compound 1 (0.039-10 mM).

FIGS. 10K-10L show changes in percent viability (FIG. 10K) and Loewe synergy scores as calculated by Combenefit software (FIG. 10L) in a CTG cell model of gastric cancer (SNUNCC19) treated with vehicle, or PI3K inhibitor idelalisib (0.078-10 mM) together with Compound 1 (0.039-10 mM).

FIGS. 10M-10N show changes in percent viability (FIG. 10M) and Loewe synergy scores as calculated by Combenefit software (FIG. 10N) in a CTG cell model of gastric cancer (SNUNCC19) treated with vehicle, or PI3K inhibitor duvelisib (0.078-10 mM) together with Compound 1 (0.039-10 mM).

FIGS. 10O-10P show changes in percent viability (FIG. 10O) and Loewe synergy scores as calculated by Combenefit software (FIG. 10P) in a CTG cell model of gastric cancer (SNUNCC19) treated with vehicle, or AKT inhibitor MK2206 (0.078-10 mM) together with Compound 1 (0.039-10 mM).

FIGS. 10Q-10R show changes in percent viability (FIG. 10Q) and Loewe synergy scores as calculated by Combenefit software (FIG. 10R) in a CTG cell model of gastric cancer (SNUNCC19) treated with vehicle, or AKT inhibitor ipataserib (0.078-10 mM) together with Compound 1 (0.039-10 mM).

FIGS. 10S-10T show changes in percent viability (FIG. 10S) and Loewe synergy scores as calculated by Combenefit software (FIG. 10T) in a CTG cell model of gastric cancer (SNUNCC19) treated with vehicle, or AKT inhibitor capivasertib (0.078-10 mM) together with Compound 1 (0.039-10 mM).

FIGS. 10U-10Z show percent viability in SNUNCC19 cells treated with Compound 1 (0.04-10 mM) in combination with GSK2636771 (GSK263) (FIG. 10U), eganelisib (FIG. 10V), idelalisib (FIG. 10W), duvelisib (FIG. 10X), MK2206 (FIG. 10Y), and ipatasertib (FIG. 10Z).

FIGS. 11A-11B show changes in percent viability (FIG. 11A) and Loewe synergy scores as calculated by Combenefit software (FIG. 11B) in a CTG cell model of pancreatic cancer (T3M4) treated with vehicle, or AKT inhibitor capivasertib (0.03-10 mM) together with Compound 1 (0.039-10 mM).

FIG. 12 provides a list of PI3K and AKT inhibitors.

FIG. 13 shows the changes in tumor volume (mm³) in mice implanted with NUGC3-74 cancer tumors upon receiving treatment with Compound 1, 50 or 100 mg/kg (QD) dosed 6 hours apart from treatment with PI3K inhibitor alpelisib, 20 mg/kg (QD×21).

FIG. 14 shows the changes in tumor volume (mm³) in eight individual mice implanted with T3M4-19 cancer tumors upon receiving treatment with Compound 1, 100 mg/kg (QD) dosed 6 hours apart from treatment with PI3K inhibitor alpelisib, 20 mg/kg (QD×13).

### DETAILED DESCRIPTION

The present invention provides compounds and methods for restoring wild-type function to mutant p53. The compounds of the present invention can bind to mutant p53 and restore the ability of the p53 mutant to bind DNA. The restoration of activity of the p53 mutant can allow for the activation of downstream effectors of p53 leading to inhibition of cancer progression. The invention further provides methods of treatment of a cancerous lesion or a tumor harboring a p53 mutation with combinations of p53 activating compounds provided herein with agents that modulate additional targets, including, for example, MDM2, PI3K, and AKT.

In some embodiments, the present disclosure provides a method comprising treating cancer by administering a combination comprising a p53 activating compound provided herein and an MDM2 inhibitor. MDM2, also known as mouse double minute 2 homolog, is a negative regulator of p53, and functions as an E3 ubiquitin ligase that targets p53 for degradation by the proteosome. MDM2 can additionally inhibit the function of p53 by binding and blocking the p53 transcriptional activation domain and facilitating the transport of p53 from the nucleus to the cytoplasm. Inhibition of the MDM2-p53 interaction can suppress each of the aforementioned mechanisms by which MDM2 negatively regulates p53.

The present disclosure also contemplates methods of treating cancer comprising administration of a p53 activating compound provided herein and a PI3K inhibitor. PI3K kinases are members of a conserved family of intracellular lipid kinases that phosphorylate the 3′-OH group on phosphatidylinositols or phosphoinositides. PI3K kinases are signaling enzymes that can relay signals from cell surface receptors to downstream effectors. The PI3K family comprises at least 15 kinases with distinct substrate specificities, expression patterns, and modes of regulation. The class I PI3K kinases (p110α, p110β, p110δ, and p110γ) can be activated by tyrosine kinases or G-protein coupled receptors to generate PIP3, which engages downstream effectors such as those in the Akt/PDK1 pathway, mTOR, the Tec family kinases, and the Rho family GTPases. The PI3K signaling pathway is known to be one of the most highly mutated in human cancers.

In some embodiments, the present disclosure provides a method comprising treating cancer by administering a combination comprising a p53 activating compound provided herein and an AKT inhibitor. AKT, also known as protein kinase B (PKB), is a growth regulator in PI3K signaling. Isoform Akt1 is a member of the PI3K/AKT/mTOR signaling pathway, and can serve to inhibit apoptotic processes and promote cell survival.

Cancer is a collection of related diseases characterized by uncontrolled proliferation of cells with the potential to metastasize throughout the body. Cancer can be classified into five broad categories including, for example: carcinomas, which can arise from cells that cover internal and external parts of the body such as the lung, breast, and colon; sarcomas, which can arise from cells that are located in bone, cartilage, fat, connective tissue, muscle, and other supportive tissues; lymphomas, which can arise in the lymph nodes and immune system tissues; leukemia, which can arise in the bone marrow and accumulate in the bloodstream; and adenomas, which can arise in the thyroid, the pituitary gland, the adrenal gland, and other glandular tissues.

Although different cancers can develop in virtually any of the body's tissues, and contain unique features, the basic processes that cause cancer can be similar in all forms of the disease. Cancer begins when a cell breaks free from the normal restraints on cell division and begins to grow and divide out of control. Genetic mutations in the cell can preclude the ability of the cell to repair damaged DNA or initiate apoptosis, and can result in uncontrolled growth and division of cells.

The ability of tumor cell populations to multiply is determined not only by the rate of cell proliferation but also by the rate of cell attrition. Programmed cell death, or apoptosis, represents a major mechanism of cellular attrition. Cancer cells can evade apoptosis through a variety of strategies, for example, through the suppression of p53 function, thereby suppressing expression of pro-apoptotic proteins.

Oncogenes and tumor suppressor genes can regulate the proliferation of cells. Genetic mutations can affect oncogenes and tumor suppressors, potentially activating or suppressing activity abnormally, further facilitating uncontrolled cell division. Whereas oncogenes assist in cellular growth, tumor suppressor genes slow cell division by repairing damaged DNA and activating apoptosis. Cellular oncogenes that can be mutated in cancer include, for example, Cdk1, Cdk2, Cdk3, Cdk4, Cdk6, EGFR, PDGFR, VEGF, HER2, Raf kinase, K-Ras, and myc. Tumor suppressor genes that can be mutated in cancer include, for example, BRCA1, BRCA2, cyclin-dependent kinase inhibitor 1C, Retinoblastoma protein (pRb), PTEN, p16, p27, p53, and p73.

### Tumor Suppressor p53.

The tumor suppressor protein p53 is a 393 amino acid transcription factor that can regulate cell growth in response to cellular stresses including, for example, UV radiation, hypoxia, oncogene activation, and DNA damage. p53 has various mechanisms for inhibiting the progression of cancer including, for example, initiation of apoptosis, maintenance of genomic stability, cell cycle arrest, induction of senescence, and inhibition of angiogenesis. Due to the critical role of p53 in tumor suppression, p53 is inactivated in almost all cancers either by direct mutation or through perturbation of associated signaling pathways involved in tumor suppression. Homozygous loss of the p53 gene occurs in almost all types of cancer, including carcinomas of the breast, colon, and lung. The presence of certain p53 mutations in several types of human cancer can correlate with less favorable patient prognosis.

In the absence of stress signals, p53 levels are maintained at low levels via the interaction of p53 with Mdm2, an E3 ubiquitin ligase. In an unstressed cell, Mdm2 can target p53 for degradation by the proteasome. Under stress conditions, the interaction between Mdm2 and p53 is disrupted, and p53 accumulates. The critical event leading to the activation of p53 is phosphorylation of the N-terminal domain of p53 by protein kinases, thereby transducing upstream stress signals. The phosphorylation of p53 leads to a conformational change, which can promote DNA binding by p53 and allow transcription of downstream effectors. The activation of p53 can induce, for example, the intrinsic apoptotic pathway, the extrinsic apoptotic pathway, cell cycle arrest, senescence, and DNA repair. p53 can activate proteins involved in the above pathways including, for example, Fas/Apo1, KILLER/DR5, Bax, Puma, Noxa, Bid, caspase-3, caspase-6, caspase-7, caspase-8, caspase-9, and p21 (WAF1). Additionally, p53 can repress the transcription of a variety of genes including, for example, c-MYC, Cyclin B, VEGF, RAD51, and hTERT.

Each chain of the p53 tetramer is composed of several functional domains including the transactivation domain (amino acids 1-100), the DNA-binding domain (amino acids 101-306), and the tetramerization domain (amino acids 307-355), which are highly mobile and largely unstructured. Most p53 cancer mutations are located in the DNA-binding core domain of the protein, which contains a central β-sandwich of anti-parallel β-sheets that serves as a basic scaffold for the DNA-binding surface. The DNA-binding surface is composed of two β-turn loops, L2 and L3, which are stabilized by a zinc ion, for example, at Arg175 and Arg248, and a loop-sheet-helix motif. Altogether, these structural elements form an extended DNA-binding surface that is rich in positively-charged amino acids, and makes specific contact with various p53 response elements.

Due to the prevalence of p53 mutations in virtually every type of cancer, the reactivation of wild type p53 function in a cancerous cell can be an effective therapy. Mutations in p53 located in the DNA-binding domain of the protein or periphery of the DNA-binding surface result in aberrant protein folding required for DNA recognition and binding. Mutations in p53 can occur, for example, at amino acids Val143, His168, Arg175, Tyr220, Gly245, Arg248, Arg249, Phe270, Arg273, and Arg282. p53 mutations that can abrogate the activity of p53 include, for example, R175H, Y220C, G245S, R248Q, R248W, R273H, and R282H. These p53 mutations can either distort the structure of the DNA-binding site or thermodynamically destabilize the folded protein at body temperature. Wild-type function of p53 mutants can be recovered by binding of the p53 mutant to a compound that can shift the folding-unfolding equilibrium towards the folded state, thereby reducing the rate of unfolding and destabilization.

Non-limiting examples of amino acids include: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val).

### Mechanism of Compounds of the Invention.

The compounds of the present invention can selectively bind to a p53 mutant and can recover wild-type activity of the p53 mutant including, for example, DNA binding function and activation of downstream targets involved in tumor suppression. In some embodiments, a compound of the invention selectively binds to the p53 Y220C mutant. The Y220C mutant is a temperature sensitive mutant, which binds to DNA at lower temperature and is denatured at body temperature. A compound of the invention can stabilize the Y220C mutant to reduce the likelihood of denaturation of the protein at body temperature.

In some embodiments, the compounds of the disclosure stabilize a mutant p53 and allows the mutant p53 to bind to DNA, thereby shifting the equilibrium of wild type and mutant p53 proteins to wild type p53. In some embodiments, the compounds of the disclosure reactivate the mutant p53 protein to provide wild type p53 activity. In some embodiments, the compounds of the disclosure reactivate the mutant p53 protein to provide pro-apoptotic p53 activity. In some embodiments, the compounds of the disclosure reactivate the mutant p53 protein to block angiogenesis. In some embodiments, the compounds of the disclosure reactivate the mutant p53 protein to induce cellular senescence. In some embodiments, the compounds of the disclosure reactivate the mutant p53 protein to induce cell cycle arrest.

In some embodiments, the compounds of the disclosure can reconform mutant p53 to a conformation of p53 that exhibits anti-cancer activity. In some embodiments, the mutant p53 is reconformed to a wild type conformation p53. In some embodiments, the mutant p53 is reconformed to a pro-apoptotic conformation of p53. In some embodiments, the mutant p53 is reconformed to a conformation of p53 that blocks angiogenesis. In some embodiments, the mutant p53 is reconformed to a conformation of p53 that induces cellular senescence. In some embodiments, the mutant p53 is reconformed to a conformation of p53 that induces cell-cycle arrest.

Located in the periphery of the p53 β-sandwich connecting β-strands S7 and S8, the aromatic ring of Y220 is an integral part of the hydrophobic core of the β-sandwich. The Y220C mutation can be highly destabilizing, due to the formation of an internal surface cavity. A compound of the invention can bind to and occupy this surface crevice to stabilize the β-sandwich, thereby restoring wild-type p53 DNA-binding activity.

To determine the ability of a compound of the invention to bind and stabilize mutant p53, assays can be employed to detect, for example, a conformational change in the p53 mutant or activation of wild-type p53 targets. Conformational changes in p53 can be measured by, for example, differential scanning fluorimetry (DSF), isothermal titration calorimetry (ITC), nuclear magnetic resonance spectrometry (NMR), or X-ray crystallography. Additionally, antibodies specific for the wild type of mutant conformation of p53 can be used to detect a conformational change via, for example, immunoprecipitation (IP), immunofluorescence (IF), or immunoblotting.

Methods used to detect the ability of the p53 mutant to bind DNA can include, for example, DNA affinity immunoblotting, modified enzyme-linked immunosorbent assay (ELISA), electrophoretic mobility shift assay (EMSA), fluorescence resonance energy transfer (FRET), homogeneous time-resolved fluorescence (HTRF), and a chromatin immunoprecipitation (ChIP) assay.

To determine whether a compound described herein is able to reactivate the transcriptional activity of p53, the activation of downstream targets in the p53 signaling cascade can be measured. Activation of p53 effector proteins can be detected by, for example, immunohistochemistry (IHC-P), reverse transcription polymerase chain reaction (RT-PCR), and western blotting. The activation of p53 can also be measured by the induction of apoptosis via the caspase cascade and using methods including, for example, Annexin V staining, TUNEL assays, pro-caspase and caspase levels, and cytochrome c levels. Another consequence of p53 activation is senescence, which can be measured using methods such as β-galactosidase staining.

A p53 mutant that can be used to determine the effectiveness of a compound of the invention to increase the DNA binding ability of a p53 mutant is a p53 truncation mutant, which contains only amino acids 94-312, encompassing the DNA-binding domain of p53. For example, the sequence of the p53 Y220C mutant used for testing compound efficacy can be:

(SEQ ID NO. 1)
SSSVPSQ KTYQGSYGFR LGFLHSGTAK SVTCTYSPAL
NKMFCQLAKT CPVQLWVDST PPPGTRVRAM AIYKQSQHMT
EVVRRCPHHE RCSDSDGLAP PQHLIRVEGN LRVEYLDDRN
TFRHSVVVPC EPPEVGSDCT TIHYNYMCNS SCMGGMNRRP
ILTIITLEDS SGNLLGRNSF EVHVCACPGR DRRTEEENLR
KKGEPHHELP PGSTKRALSN NT

A compound of the invention can increase the ability of a p53 mutant to bind DNA by at least or up to about 0.1%, at least or up to about 0.2%, at least or up to about 0.3%, at least or up to about 0.4%, at least or up to about 0.5%, at least or up to about 0.6%, at least or up to about 0.7%, at least or up to about 0.8%, at least or up to about 0.9%, at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 110%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 21%, at least or up to about 22%, at least or up to about 23%, at least or up to about 24%, at least or up to about 25%, at least or up to about 26%, at least or up to about 27%, at least or up to about 28%, at least or up to about 29%, at least or up to about 30%, at least or up to about 31%, at least or up to about 32%, at least or up to about 33%, at least or up to about 34%, at least or up to about 35%, at least or up to about 36%, at least or up to about 37%, at least or up to about 38%, at least or up to about 39%, at least or up to about 40%, at least or up to about 41%, at least or up to about 42%, at least or up to about 43%, at least or up to about 44%, at least or up to about 45%, at least or up to about 46%, at least or up to about 47%, at least or up to about 48%, at least or up to about 49%, at least or up to about 50%, at least or up to about 51%, at least or up to about 52%, at least or up to about 53%, at least or up to about 54%, at least or up to about 55%, at least or up to about 56%, at least or up to about 57%, at least or up to about 58%, at least or up to about 59%, at least or up to about 60%, at least or up to about 61%, at least or up to about 62%, at least or up to about 63%, at least or up to about 64%, at least or up to about 65%, at least or up to about 66%, at least or up to about 67%, at least or up to about 68%, at least or up to about 69%, at least or up to about 70%, at least or up to about 71%, at least or up to about 72%, at least or up to about 73%, at least or up to about 74%, at least or up to about 75%, at least or up to about 76%, at least or up to about 77%, at least or up to about 78%, at least or up to about 79%, at least or up to about 80%, at least or up to about 81%, at least or up to about 82%, at least or up to about 83%, at least or up to about 84%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, at least or up to about 100%, at least or up to about 125%, at least or up to about 150%, at least or up to about 175%, at least or up to about 200%, at least or up to about 225%, or at least or up to about 250% as compared to the ability of the p53 mutant to bind DNA in the absence of a compound of the invention.

A compound described herein can increase the activity of the p53 mutant that is, for example, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 16-fold, at least or up to about 17-fold, at least or up to about 18-fold, at least or up to about 19-fold, at least or up to about 20-fold, at least or up to about 25-fold, at least or up to about 30-fold, at least or up to about 35-fold, at least or up to about 40-fold, at least or up to about 45-fold, at least or up to about 50-fold, at least or up to about 55-fold, at least or up to about 60-fold, at least or up to about 65-fold, at least or up to about 70-fold, at least or up to about 75-fold, at least or up to about 80-fold, at least or up to about 85-fold, at least or up to about 90-fold, at least or up to about 95-fold, at least or up to about 100-fold, at least or up to about 110-fold, at least or up to about 120-fold, at least or up to about 130-fold, at least or up to about 140-fold, at least or up to about 150-fold, at least or up to about 160-fold, at least or up to about 170-fold, at least or up to about 180-fold, at least or up to about 190-fold, at least or up to about 200-fold, at least or up to about 250-fold, at least or up to about 300-fold, at least or up to about 350-fold, at least or up to about 400-fold, at least or up to about 450-fold, at least or up to about 500-fold, at least or up to about 550-fold, at least or up to about 600-fold, at least or up to about 650-fold, at least or up to about 700-fold, at least or up to about 750-fold, at least or up to about 800-fold, at least or up to about 850-fold, at least or up to about 900-fold, at least or up to about 950-fold, at least or up to about 1,000-fold, at least or up to about 1,500-fold, at least or up to about 2,000-fold, at least or up to about 3,000-fold, at least or up to about 4,000-fold, at least or up to about 5,000-fold, at least or up to about 6,000-fold, at least or up to about 7,000-fold, at least or up to about 8,000-fold, at least or up to about 9,000-fold, or at least or up to about 10,000-fold greater than the activity of the p53 mutant in the absence of the compound.

A compound of the invention can be used, for example, to induce apoptosis, cell cycle arrest, or senescence in a cell. In some embodiments, the cell is a cancer cell. In some embodiments, the cell carries a mutation in p53.

### Compounds of the Invention.

In some embodiments, a compound of the disclosure comprises a substituted heterocyclyl group, wherein the compound binds a mutant p53 protein and increases wild-type p53 activity of the mutant protein. In some embodiments, a compound of the disclosure comprises a heterocyclyl group comprising a halo substituent, wherein the compound binds a mutant p53 protein and increases wild-type p53 activity of the mutant protein. In some embodiments, the compound further comprises a 5,6-fused bicyclic ring. In some embodiments, the 5,6-fused bicyclic ring is an indole group. In some embodiments, the 5,6-fused bicyclic ring is a benzothiophene group. In some embodiments, the 5,6-fused bicyclic ring is an indazole group. In some embodiments, the 5,6-fused bicyclic ring is an imidazopyridine group. In some embodiments, the 5,6-fused bicyclic ring is an imidazo[1,2-a]pyridine group. In some embodiments, the 5,6-fused bicyclic ring is an indolizine group. In some embodiments, the 5,6-fused bicyclic ring is a pyrazolopyridine group. In some embodiments, the 5,6-fused bicyclic ring is an pyrazolo[1,5-a]pyridine.

In some embodiments, the indole group has a 2,2,2,-trifluoroethyl substituent at a 1-position of the indole group. In some embodiments, the indole group has a 2,2,2,-trifluoroethyl substituent at a 1-position of the benzothiophene group. In some embodiments, the indole group has a 2,2,2,-trifluoroethyl substituent at a 3-position of the imidazo[1,2-a]pyridine group. In some embodiments, the indole group has a 2,2,2,-trifluoroethyl substituent at a 3-position of the indolizine group. In some embodiments, the indole group has a 2,2,2,-trifluoroethyl substituent at a 3-position of the pyrazolo[1,5-a]pyridine group.

In some embodiments, the indole group has a trifluoromethylthio substituent at a 1-position of the indole group. In some embodiments, the indole group has a trifluoromethylthio substituent at a 1-position of the benzothiophene group. In some embodiments, the indole group has a trifluoromethylthio substituent at a 3-position of the imidazo[1,2-a]pyridine group. In some embodiments, the indole group has a trifluoromethylthio substituent at a 3-position of the indolizine group. In some embodiments, the indole group has a trifluoromethylthio substituent at a 3-position of the pyrazolo[1,5-a]pyridine group.

In some embodiments, the indole group has a propargyl substituent at a 2-position of the indole group. In some embodiments, the indole group has a propargyl substituent at a 2-position of the benzothiophene group. In some embodiments, the indole group has a propargyl substituent at a 2-position of the imidazo[1,2-a]pyridine group. In some embodiments, the indole group has a propargyl substituent at a 2-position of the indolizine group. In some embodiments, the indole group has a propargyl substituent at a 2-position of the pyrazolo[1,5-a]pyridine group.

In some embodiments, the propargyl substituent is attached to the indole group via an sp carbon atom of the propargyl substituent. In some embodiments, the propargyl substituent is attached to a nitrogen atom of an aniline group via a methylene group of the propargyl substituent. In some embodiments, the indole group comprises an amino substituent at a 4-position of the indole group. In some embodiments, the amino substituent is attached to the heterocyclyl group. In some embodiments, the heterocyclyl group is a piperidine group. In some embodiments, the halo substituent is a fluoro group. In some embodiments, the halo substituent is a chloro group. In some embodiments, the compound has oral bioavailability that is at least about 50% greater than that of an analogous compound that lacks the halo substituent on the heterocyclyl group.

In some embodiments, the compound is of the formula:

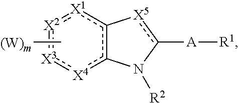

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each W is independently -Q¹-N(R³)R⁴, -Q¹-OR⁴, or -Q¹-R⁴;
   wherein at least one of X¹, X², X³, and X⁴ is a carbon atom connected to Q¹;
A is a linking group;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, halogen, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is independently alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,

or a pharmaceutically-acceptable salt thereof.

In some embodiments, A is alkylene, alkenylene, or alkynylene, each of which is substituted or unsubstituted. In some embodiments, A is alkylene. In some embodiments, A is alkenylene. In some embodiments, A is alkynylene.

In some embodiments, A is arylene, heteroarylene, or heterocyclylene, each of which is substituted or unsubstituted. In some embodiments, A is arylene. In some embodiments, A is heteroarylene. In some embodiments, A is heterocyclylene. In some embodiments, A is substituted arylene. In some embodiments, A is substituted heteroarylene. In some embodiments, A is substituted heterocyclylene.

In some embodiments, R¹ is alkyl, alkenyl, —C(O)R¹⁶, —C(O)OR¹⁶, or —C(O)NR¹⁶R¹⁷, each of which is unsubstituted or substituted. In some embodiments, R¹ is substituted alkyl. In some embodiments, R¹ is alkyl substituted with NR¹⁶R¹⁷.

In some embodiments, the compound of the formula is:

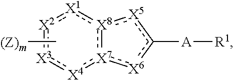

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
X⁶ is CR², NR², O, or S;
X⁷ is C or N;
X⁸ is C or N;
each Z is independently -Q¹-N(R³)J, -Q¹-O-J, or -Q¹-J;
   wherein at least one of X¹, X², X³, and X⁴ is a carbon atom connected to Q¹;
A is a linking group;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
each J is independently a cyclic group;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and J together with the nitrogen atom to which R³ and J are bound form a ring, wherein the ring is substituted or unsubstituted;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound of the formula is:

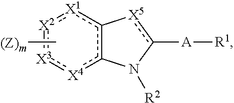

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each Z is independently -Q¹-N(R³)J, -Q¹-O-J, or -Q¹-J;
wherein at least one of X¹, X², X³, and X⁴ is a carbon atom connected to Q¹;
A is a linking group;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
each J is independently a cyclic group;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and J together with the nitrogen atom to which R³ and J are bound form a ring, wherein the ring is substituted or unsubstituted;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, a compound of the invention is a compound of the formula

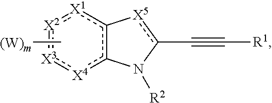

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each W is independently -Q¹-N(R³)R⁴, -Q¹-OR⁴, or -Q¹-R⁴;
   wherein at least one of X¹, X², X³, and X⁴ is a carbon atom connected to Q¹;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ and R⁴ is independently alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or C(O)R²³, —C(O)OR²³, —C(O)NR²³R²⁴, —OR²³, —SR²³, —NR²³R²⁴, —NR²³C(O)R²⁴, —OC(O)R²³, hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,

or a pharmaceutically-acceptable salt thereof.

In some embodiments, the compound is of the formula:

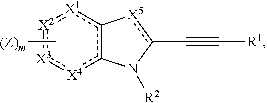

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each Z is independently -Q¹-N(R³)J, -Q¹-O-J, or -Q¹-J;
   wherein at least one of X¹, X², X³, and X⁴ is a carbon atom connected to Q¹;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
each J is independently a cyclic group;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ is independently alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and J together with the nitrogen atom to which R³ and J are bound form a ring, wherein the ring is substituted or unsubstituted;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the pattern of dashed bonds is chosen to provide an aromatic system, for example, an indole, a benzothiophene, a indolizine, a pyrrolopyridine, a pyrrolopyrimidine, or a pyrrolopyrazine.

In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Q¹ is a bond. In some embodiments, Q¹ is C₁-alkylene. In some embodiments, m is 1. In some embodiments, m is 2. In some embodiments, m is 3. In some embodiments, m is 4.

In some embodiments, R¹ is alkyl, alkenyl, each of which is unsubstituted or substituted, or —C(O)R¹⁶, —C(O)OR¹⁶, or —C(O)NR¹⁶R¹⁷. In some embodiments, R¹ is substituted alkyl. In some embodiments, R¹ is alkyl substituted with NR¹⁶R¹⁷.

In some embodiments, J is aryl, heteroaryl, or heterocyclyl, each of which is substituted or unsubstituted. In some embodiments, J is substituted aryl. In some embodiments, J is aryl substituted with fluoro-. In some embodiments, J is aryl substituted with chloro-. In some embodiments, J is substituted heteroaryl, In some embodiments, J is heteroaryl substituted with fluoro-. In some embodiments, J is heteroaryl substituted with chloro-. In some embodiments, J is substituted heterocyclyl. In some embodiments, J is heterocyclyl substituted with fluoro-. In some embodiments, J is heterocyclyl substituted with chloro-.

In some embodiments, J is piperidinyl, piperazinyl, tetahydropyranyl, morpholinyl, or pyrrolidinyl, each of which is independently substituted or unsubstituted. In some embodiments, J is piperidinyl, piperazinyl, tetahydropyranyl, morpholinyl, or pyrrolidinyl, each of which is independently substituted with at least halo-. In some embodiments, J is piperidinyl substituted with halo-. In some embodiments, J is methylpiperidinyl substituted with halo-. In some embodiments, J is 3-fluoro-1-methylpiperidinyl. In some embodiments, J is 3-fluoro-1-(2-hydroxy-3-methoxypropyl)piperidinyl. In some embodiments, J is tetrahydropyranyl substituted with at least halo-.

In some embodiments, each R¹⁶ and R¹⁷ is independently alkyl, alkenyl, aryl, heteroaryl, heterocyclyl, each of which is independently substituted or unsubstituted, or hydrogen. In some embodiments, R¹⁶ is hydrogen or alkyl. In some embodiments, R¹⁷ is aryl, heteroaryl, or heterocyclyl, each of which is independently substituted or unsubstituted. In some embodiments, R¹⁷ is substituted aryl. In some embodiments, R¹⁷ is substituted phenyl. In some embodiments, R¹⁷ is phenyl substituted with a sulfoxide group, sulfonyl group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with at least methoxy. In some embodiments, R¹⁷ is phenyl substituted with a substituted sulfoxide group. In some embodiments, R¹⁷ is phenyl substituted with a carboxyl group. In some embodiments, R¹⁷ is phenyl substituted with a substituted amide group.

In some embodiments, the compound is of the formula:

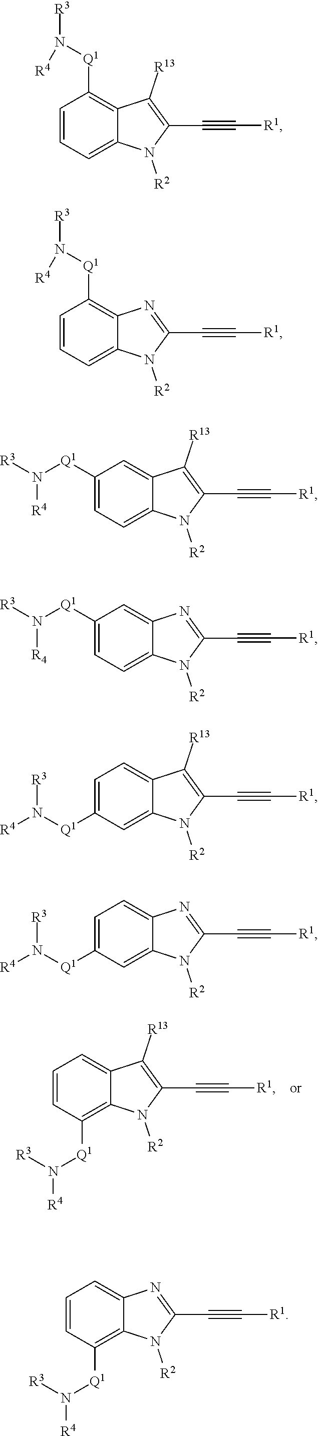

In some embodiments, Q¹ is C═, C═S, C═CR¹⁴R¹⁵, C═NR¹⁴, alkylene, alkenylene, or alkynylene, each of which is independently substituted or unsubstituted, or a bond. In some embodiments, Q¹ is alkylene, alkenylene, or alkynylene. In some embodiments, Q¹ is C₁-alkylene or a bond. In some embodiments, Q¹ is C₁-alkylene. In some embodiments, Q¹ is a bond.

In some embodiments, W is -Q¹-N(R³)R⁴. In some embodiments, W is -Q¹-OR⁴. In some embodiments, W is -Q¹-R⁴.

In some embodiments, Z is -Q¹-N(R³)J. In some embodiments, Z is -Q¹-O-J. In some embodiments, Z is -Q¹-J.

In some embodiments, R² is hydrogen or alkyl. In some embodiments, R² is alkyl. In some embodiments, R2 is substituted C₁-C₅-alkyl. In some embodiments, R² is trifluoroethyl. In some embodiments, R² is cycloalkyl. In some embodiments, R² is cyclopropyl.

In some embodiments, R¹³ is alkyl, alkenyl, hydrogen, or halogen. In some embodiments, R¹³ is hydrogen.

In some embodiments, R² is C₁-C₅-alkyl, and R¹³ is C₁-C₅-alkyl. In some embodiments, R2 is C₁-C₅-alkyl, and R¹³ is hydrogen. In some embodiments, R2 is substituted C₁-C₅-alkylene. In some embodiments, R² is methyl, ethyl, propyl, iso-propyl, butyl, or tert-butyl, each of which is substituted or unsubstituted. In some embodiments, R¹³ is methyl, ethyl, propyl, iso-propyl, butyl or tert-butyl. In some embodiments, R2 is hydrogen, and R¹³ is hydrogen. In some embodiments, R2 is trifluoroethyl, and R¹³ is hydrogen.

In some embodiments, the compound is of the formula:

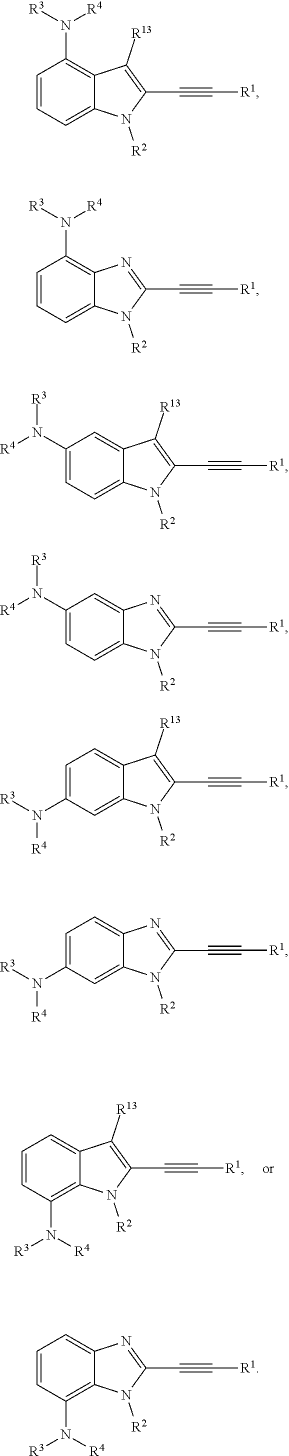

In some embodiments, the compound is of the formula:

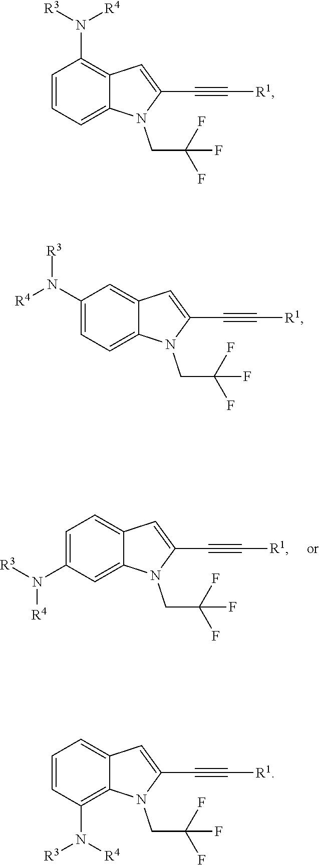

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkylene, alkenyl, alkenylene, alkynyl, aryl, heteroaryl, or heterocyclyl, each of which is independently substituted or unsubstituted. In some embodiments, each R³ and R⁴ is independently substituted or unsubstituted C₁-C₆-alkylene. In some embodiments, R³ is H, and R⁴ is substituted or unsubstituted C₁-C₄ alkylene. In some embodiments, R³ is H, and R⁴ is substituted or unsubstituted heterocyclyl. In some embodiments, R³ is H, and R⁴ is substituted or unsubstituted piperidinyl. In some embodiments, R³ is H, and R⁴ is substituted or unsubstituted cycloalkyl. In some embodiments, R³ is H, and R⁴ is cycloalkyl substituted with an amino group. In some embodiments, R³ is H, and R⁴ is substituted or unsubstituted cyclobutyl. In some embodiments, R³ is H, and R⁴ is cyclobutyl substituted with an amino group. In some embodiments, R³ is H, and R⁴ is substituted or unsubstituted cyclohexyl. In some embodiments, R³ is H, and R⁴ is cyclohexyl substituted with an amino group.

In some embodiments, the compound is of the formula:

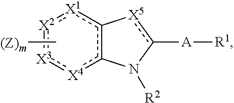

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each Z is independently -Q¹-N(R³)J, -Q¹-O-J, or -Q¹-J;
   wherein at least one of X¹, X², X³, and X⁴ is a carbon atom connected to Q¹;
A is C₃₋₉cycloalkylene, C₆₋₁₀arylene, C₁₋₉heterocyclylene, C₁₋₆alkylene, C₂₋₆alkenylene, or C₂₋₆alkynylene each of which is independently unsubstituted or substituted with 1 to 7 R^{A};
each Q¹ is independently C═O, C═S, C═CR¹⁴R¹⁵, C═NR¹⁴, or a bond; or C₁₋₆alkylene, C₂₋₆alkenylene, or C₂₋₆alkynylene, each of which is independently unsubstituted or substituted with 1 to 7 R^{B};
m is 1, 2, 3, or 4;
each J is independently C₃₋₉cycloalkyl, C₆₋₁₀aryl, C₁₋₉heteroaryl, or C₁₋₉heterocyclyl, each of which is independently unsubstituted or substituted with 1 to 7 R^{C};
R¹ is C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{D}, or —(CH₂)_{w}C(O)R¹⁶, —(CH₂)_{w}C(O)OR¹⁶, —(CH₂)_{w}C(O)NR¹⁶R¹⁷, —(CH₂)_{w}OR¹⁶, —(CH₂)_{w}SR¹⁶, —(CH₂)_{w}NR¹⁶R¹⁷, —(CH₂)_{w}NR¹⁶C(O)R¹⁷, —(CH₂)_{w}OC(O)R¹⁶, —(CH₂)_{w}P(O)R¹⁶R¹⁷, or —(CH₂)_{w}SiR¹⁶R¹⁷R¹⁹, wherein w is 0, 1, 2, or 3;
each R³ is independently C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{E}, or —C(O)R¹⁹, —C(O)OR¹⁹, —C(O)NR¹⁹R²⁰, —SOR¹⁹, —SO₂R¹⁹, or hydrogen, or R³ and J together with the nitrogen atom to which R³ and J are bound form a ring, wherein the ring is unsubstituted or substituted with 1 to 7 R^{E};
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{F}, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{G}, or C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{H};
each R^{A}, R^{B}, R^{C}, R^{D}, R^{E}, R^{F}, R^{G}, and R^{H} is independently selected from —CN, halogen, —C(O)R²³, —C(O)OR²³, —NR²³R²⁴, —OR²³, —C(O)NR²³R²⁴, —SR²³, —S(O)R²³, —SO₂R²³, —NR²³C(O)R²³, and —OC(O)R²³; or C₁₋₆alkyl, C₁₋₆heteroalkyl, C₆₋₁₀aryl, and C₁₋₉heterocyclyl, C₁₋₆alkylene-C₆₋₁₀aryl, and C₁₋₆alkylene-C₁₋₉heterocyclyl, each of which is independently unsubstituted or substituted with —CN, halogen, methyl, ethyl, propyl, butyl, —C(O)R²³, —C(O)OR²³, —C(O)NR²³R²⁴, —OR²³, —SR²³, —S(O)R²³, —S(O)₂R²³, —NR²³R²⁴, —NR²³C(O)R²⁴, or —OC(O)R²³; and
each R²³ and R²⁴ is independently hydrogen, or C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with —CN, halogen, C₁₋₆alkyl, or C₁₋₆haloalkyl,
   or a pharmaceutically-acceptable salt thereof.

In some embodiments, each Z is -Q¹-N(R³)J.

In some embodiments, A is C₆₋₁₀arylene, C₁₋₉heterocyclylene, or C₂₋₆alkynylene, each of which is independently unsubstituted or substituted with 1 to 7 R^{A}. In some embodiments, A is C₆₋₁₀arylene. In some embodiments, A is phenylene that is unsubstituted or substituted with 1 to 4 R^{A}. In some embodiments, A is pyrid-diyl that is unsubstituted or substituted with 1 to 3 R^{A}. In some embodiments, A is pyrid-2,6-diyl that is unsubstituted or substituted with 1 to 3 R^{A}. In some embodiments, A is pyrid-2,5-diyl that is unsubstituted or substituted with 1 to 3 R^{A}. In some embodiments, A is pyrid-2,4-diyl that is unsubstituted or substituted with 1 to 3 R^{A}. In some embodiments, A is pyrid-2,3-diyl that is unsubstituted or substituted with 1 to 3 R^{A}. In some embodiments, A is pyrid-3,5-diyl that is unsubstituted or substituted with 1 to 3 R^{A}. In some embodiments, A is pyrid-3,4-diyl that is unsubstituted or substituted with 1 to 3 R^{A}.

In some embodiments, A is C₁₋₉heterocyclylene. In some embodiments, A is 5-membered heteroaryl with 1, 2, or 3 heteroatoms selected from N, O, or S. In some embodiments, A is 1H-imidazoldiyl that is unsubstituted or substituted with 1 or 2 R^{A}. In some embodiments, A is 1H-imidazol-2,5-diyl that is unsubstituted or substituted with 1 or 2 R^{A}. In some embodiments, A is 1H-pyrazoldiyl that is unsubstituted or substituted with 1 or 2 R^{A}. In some embodiments, A is 1H-pyrazol-3,5-diyl that is unsubstituted or substituted with 1 or 2 R^{A}. In some embodiments, A is 1H-pyrazol-1,4-diyl that is unsubstituted or substituted with 1 or 2 R^{A}. In some embodiments, A is thiophendiyl that is unsubstituted or substituted with 1 or 2 R^{A}. In some embodiments, A is thiophen-2,5-diyl that is unsubstituted or substituted with 1 or 2 R^{A}. In some embodiments, A is thiazoldiyl that is unsubstituted or substituted with 1 or 2 R^{A}. In some embodiments, A is thiazol-2,5-diyl that is unsubstituted or substituted with 1 or 2 R^{A}. In some embodiments, A is thiazol-4,5-diyl that is unsubstituted or substituted with 1 or 2 R^{A}. In some embodiments, A is 1,3,5-thiadiazoldiyl that is unsubstituted or substituted with 1 or 2 R^{A}. In some embodiments, A is 1,3,5-thiadiazol-2,5-diyl that is unsubstituted or substituted with 1 or 2 R^{A}.

In some embodiments, A is C₂₋₆alkynylene. In some embodiments, A is ethyndiyl.

In some embodiments, each Q¹ is independently a bond, or C₁₋₆alkylene that is independently unsubstituted or substituted with 1 to 7 R^{B}. In some embodiments, m is 1. In some embodiments, Q¹ is a bond.

In some embodiments, R¹ is —(CH₂)_{w}NR¹⁶R¹⁷ or —(CH₂)_{w}NR¹⁶C(O)R¹⁷, wherein w is 0 or 1. In some embodiments, R¹ is —CH₂NR¹⁶R¹⁷.

In some embodiments, the compound is of the formula:

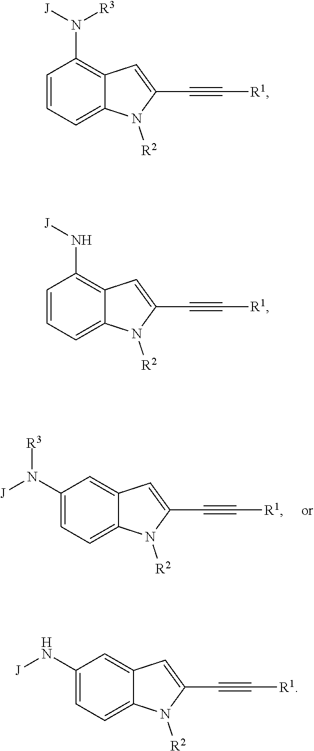

In some embodiments, the compound is of the formula:

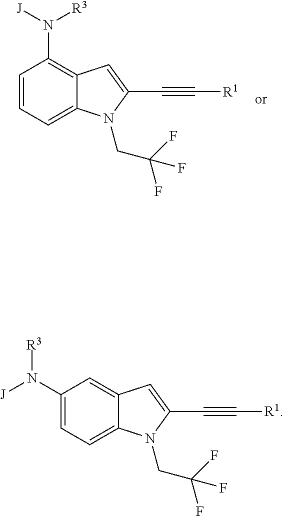

R¹ can be a group substituted with one or more substituents selected from a hydroxyl group, sulfhydryl group, halogens, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group. In some embodiments, R¹ is alkyl, alkenyl, —C(O)R¹⁶, —C(O)OR¹⁶, or —C(O)NR¹⁶R¹⁷.

In some embodiments, R¹ is substituted or unsubstituted C₁-C₃ alkyl. In some embodiments, R¹ is C₁-C₃-alkyl substituted with an amine group. In some embodiments, R¹ is C₁-alkyl substituted with NR¹⁶R¹⁷. In some embodiments, each R¹⁶ and R¹⁷ is independently aryl, heteroaryl, or heterocyclyl, each of which is independently substituted or unsubstituted, or hydrogen. In some embodiments, R¹⁶ is H, and R¹⁷ is substituted aryl. In some embodiments, R¹⁶ is H, and R¹⁷ is substituted phenyl. In some embodiments, R¹⁶ is H, and R¹⁷ is phenyl substituted with alkyl, alkoxy, halo, sulfonamide, a sulfone, or a carboxy group. In some embodiments, R¹⁶ is H, and R¹⁷ is phenyl substituted with alkyl, alkoxy, halo, sulfonamide, a sulfone, or a carbamoyl group. In some embodiments, R¹⁶ is H, and R¹⁷ is phenyl substituted with 1, 2, 3 groups independently selected from methoxy, methanesulfonyl, and methylcarbamoyl. In some embodiments, R¹⁶ is H, and R¹⁷ is substituted heteroaryl. In some embodiments, R¹⁶ is H, and R¹⁷ is substituted heterocyclyl.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is C₁-alkylene, R¹⁶ is aryl, and R¹⁷ is alkyl. In some embodiments, Q¹ is C₁-alkylene, R¹⁶ is aryl, and R¹⁷ is hydrogen. In some embodiments, Q¹ is C₁-alkylene, R¹⁶ is heteroaryl, and R¹⁷ is alkyl. In some embodiments, Q¹ is C₁-alkylene, R¹⁶ is heteroaryl, and R¹⁷ is hydrogen. In some embodiments, Q¹ is C₁-alkylene, R¹⁶ is substituted heteroaryl, and R¹⁷ is hydrogen. In some embodiments, Q¹ is C₁-alkylene, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano.

In some embodiments, R² is alkyl, and R¹³ is alkyl, each of which is substituted or substituted. In some embodiments, R2 is hydrogen, and R¹³ is unsubstituted or substituted alkyl. In some embodiments, R² is methyl, ethyl, propyl, iso-propyl, butyl, or tert-butyl, each of which is substituted or unsubstituted. In some embodiments, R¹³ is methyl, ethyl, propyl, iso-propyl, butyl or tert-butyl. In some embodiments, R² is hydrogen, and R¹³ is hydrogen. In some embodiments, R² is hydrogen, and R¹³ is alkyl. In some embodiments, R² is trifluoroethyl, and R¹³ is hydrogen.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen, and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen. In some embodiments, R³ is alkyl, alkenyl, alkynyl, aryl, heteroaryl, or heterocyclyl, each of which is independently substituted or unsubstituted, or hydrogen. In some embodiments, R³ is substituted alkyl. In some embodiments, R³ is H.

In some embodiments, R³ is H, and R⁴ is unsubstituted or substituted alkyl. In some embodiments, R³ is H, and R⁴ is unsubstituted or substituted cycloalkyl. In some embodiments, R³ is H, and R⁴ is substituted cyclohexyl. In some embodiments, R³ is H, and R⁴ is substituted cyclobutyl.

In some embodiments, at least one of R³ and R⁴ is alkyl, alkylene, alkenyl, alkenylene, alkynyl, aryl, heteroaryl, or heterocyclyl, each of which is substituted at least with halo-. In some embodiments, R⁴ or J is cycloalkyl, aryl, heteroaryl, or heterocyclyl, each of which is independently substituted or unsubstituted. In some embodiments, R⁴ or J is substituted or unsubstituted aryl. In some embodiments, R⁴ or J is substituted or unsubstituted phenyl. In some embodiments, R⁴ or J is substituted or unsubstituted cycloalkyl. In some embodiments, R⁴ or J is substituted or unsubstituted cyclopropyl. In some embodiments, R⁴ or J is substituted cyclopropyl. In some embodiments, R⁴ or J is substituted cyclohexyl. In some embodiments, R⁴ or J is cyclohexyl substituted with an amino group.

In some embodiments, R³ is H, and R⁴ or J is unsubstituted or substituted heterocyclyl. In some embodiments, R⁴ or J is heterocyclyl. In some embodiments, R⁴ or J is piperidinyl, piperazinyl, tetahydropyranyl, morpholinyl, or pyrrolidinyl, each of which is independently substituted or unsubstituted. In some embodiments, R³ is H, and R⁴ or J is substituted piperidinyl. In some embodiments, R³ is H, and R⁴ or J is piperidine substituted with alkyl, carboxy, heterocyclyl, or an amide group. In some embodiments, R³ is H, and R⁴ or J is unsubstituted or substituted methyl piperidinyl. In some embodiments, R³ is H, and R⁴ or J is 3-fluoro-1-methylpiperidinyl. In some embodiments, R³ is H, and R⁴ or J is piperidinyl substituted with methoxypropanol. In some embodiments, R³ is H, and R⁴ or J is 3-fluoro-1-(2-hydroxy-3-methoxypropyl)piperidinyl. In some embodiments, R³ is H, and R⁴ or J is unsubstituted or substituted tetrahydropyranyl. In some embodiments, R³ is H, and R⁴ or J is unsubstituted tetrahydropyranyl. In some embodiments, R³ is H, and R⁴ or J is tetrahydropyranyl substituted with alkyl. In some embodiments, R³ is H, and R⁴ or J is tetrahydrothiopyran-1,1-diooxide.

In some embodiments, R³ is H, and J is unsubstituted or substituted heterocyclyl. In some embodiments, J is heterocyclyl. In some embodiments, J is piperidinyl, piperazinyl, tetahydropyranyl, morpholinyl, or pyrrolidinyl, each of which is independently substituted or unsubstituted. In some embodiments, R³ is H, and J is substituted piperidinyl. In some embodiments, R³ is H, and J is piperidine substituted with alkyl, carboxy, heterocyclyl, or an amide group. In some embodiments, R³ is H, and J is unsubstituted or substituted methyl piperidinyl. In some embodiments, R³ is H, and J is 3-fluoro-1-methylpiperidinyl. In some embodiments, R³ is H, and J is piperidinyl substituted with methoxypropanol. In some embodiments, R³ is H, and J is 3-fluoro-1-(2-hydroxy-3-methoxypropyl)piperidinyl. In some embodiments, R³ is H, and J is unsubstituted or substituted tetrahydropyranyl. In some embodiments, R³ is H, and J is unsubstituted tetrahydropyranyl. In some embodiments, R³ is H, and J is tetrahydropyranyl substituted with alkyl. In some embodiments, R³ is H, and J is tetrahydrothiopyran-1,1-diooxide.

In some embodiments, R³ is H, and R⁴ is unsubstituted or substituted heterocyclyl. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted. In some embodiments, R³ is H, and R⁴ is substituted piperidinyl. In some embodiments, R³ is H, and R⁴ is piperidine substituted with alkyl, carboxy, heterocyclyl, or an amide group. In some embodiments, R³ is H, and R⁴ is unsubstituted or substituted methyl piperidinyl. In some embodiments, R³ is H, and R⁴ is 3-fluoro-1-methylpiperidinyl. In some embodiments, R³ is H, and R⁴ is piperidinyl substituted with methoxypropanol. In some embodiments, R³ is H, and R⁴ is 3-fluoro-1-(2-hydroxy-3-methoxypropyl)piperidinyl. In some embodiments, R³ is H, and R⁴ is unsubstituted or substituted tetrahydropyranyl. In some embodiments, R³ is H, and R⁴ is unsubstituted tetrahydropyranyl. In some embodiments, R³ is H, and R⁴ is tetrahydropyranyl substituted with alkyl. In some embodiments, R³ is H, and R⁴ is tetrahydrothiopyran-1,1-diooxide.

In some embodiments, R⁴ or J is cycloalkyl, aryl, heteroaryl, or heterocyclyl, each of which is substituted at least with halo-. In some embodiments, R⁴ or J is C₄-C₆-cycloalkyl substituted with at least halo-. In some embodiments, R⁴ or J is cyclohexyl substituted with at least halo-. In some embodiments, R⁴ or J is aryl substituted with at least halo-. In some embodiments, R⁴ or J is phenyl substituted with at least halo-. In some embodiments, R⁴ or J is aryl substituted with fluoro-. In some embodiments, R⁴ or J is phenyl substituted with fluoro-. In some embodiments, R⁴ or J is aryl substituted with chloro-. In some embodiments, R⁴ or J is phenyl substituted with chloro-. In some embodiments, R⁴ or J is heteroaryl substituted with at least halo-. In some embodiments, R⁴ or J is heteroaryl substituted with fluoro-. In some embodiments, R⁴ or J is heteroaryl substituted with chloro-. In some embodiments, R⁴ or J is C₄-C₆-heterocyclyl substituted with at least halo-. In some embodiments, R⁴ or J is heterocyclyl substituted with fluoro-. In some embodiments, R⁴ or J is heterocyclyl substituted with chloro-. In some embodiments, J is a cyclic group that is substituted or unsubstituted; R¹ is alkyl, or alkenyl, each of which is unsubstituted or substituted, —C(O)R¹⁶, —C(O)OR¹⁶, or —C(O)NR¹⁶R¹⁷; R² is substituted or unsubstituted alkyl; and R³ is H.

In some embodiments, R⁴ is cycloalkyl, aryl, heteroaryl, or heterocyclyl, each of which is substituted at least with halo-. In some embodiments, R⁴ is C₄-C₆-cycloalkyl substituted with at least halo-. In some embodiments, R⁴ is cyclohexyl substituted with at least halo-. In some embodiments, R⁴ is aryl substituted with at least halo-. In some embodiments, R⁴ is phenyl substituted with at least halo-. In some embodiments, R⁴ is aryl substituted with fluoro-. In some embodiments, R⁴ is phenyl substituted with fluoro-. In some embodiments, R⁴ is aryl substituted with chloro-. In some embodiments, R⁴ is phenyl substituted with chloro-. In some embodiments, R⁴ heteroaryl substituted with at least halo-. In some embodiments, R⁴ is heteroaryl substituted with fluoro-. In some embodiments, R⁴ is heteroaryl substituted with chloro-. In some embodiments, R⁴ is C₄-C₆-heterocyclyl substituted with at least halo-. In some embodiments, R⁴ is heterocyclyl substituted with fluoro-. In some embodiments, R⁴ is heterocyclyl substituted with fluoro- and methyl. In some embodiments, R⁴ is 6-membered heterocyclyl substituted with fluoro- and methyl. In some embodiments, R⁴ is heterocyclyl substituted with chloro-. In some embodiments, R⁴ is a cyclic group that is substituted or unsubstituted; R¹ is alkyl, or alkenyl, each of which is unsubstituted or substituted, —C(O)R¹⁶, —C(O)OR¹⁶, or —C(O)NR¹⁶R¹⁷; R² is substituted or unsubstituted alkyl; and R³ is H.

In some embodiments, J is cycloalkyl, aryl, heteroaryl, or heterocyclyl, each of which is substituted at least with halo-. In some embodiments, J is C₄-C₆-cycloalkyl substituted with at least halo-. In some embodiments, J is cyclohexyl substituted with at least halo-. In some embodiments, J is aryl substituted with at least halo-. In some embodiments, J is phenyl substituted with at least halo-. In some embodiments, J is aryl substituted with fluoro-. In some embodiments, J is phenyl substituted with fluoro-. In some embodiments, J is aryl substituted with chloro-. In some embodiments, J is phenyl substituted with chloro-. In some embodiments, J heteroaryl substituted with at least halo-. In some embodiments, J is heteroaryl substituted with fluoro-. In some embodiments, J is heteroaryl substituted with chloro-. In some embodiments, J is C₄-C₆-heterocyclyl substituted with at least halo-. In some embodiments, J is heterocyclyl substituted with fluoro-. In some embodiments, J is heterocyclyl substituted with fluoro- and methyl. In some embodiments, J is 6-membered heterocyclyl substituted with fluoro- and methyl. In some embodiments, J is heterocyclyl substituted with chloro-. In some embodiments, J is a cyclic group that is substituted or unsubstituted; R¹ is alkyl, or alkenyl, each of which is unsubstituted or substituted, —C(O)R¹⁶, —C(O)OR¹⁶, or —C(O)NR¹⁶R¹⁷; R² is substituted or unsubstituted alkyl; and R³ is H.

In some embodiments, J is C₃₋₉cycloalkyl or C₁₋₉heterocyclyl, each of which is independently unsubstituted or substituted with 1 to 7 R^{C}. In some embodiments, J is 6-membered cycloalkyl or 6-membered heterocycloalkyl, each of which is independently unsubstituted or substituted with 1 to 7 R^{C}. In some embodiments, J is cyclohexyl or piperdinyl, each of which is independently unsubstituted or substituted with 1 to 7 R^{C}; and R³ is H.

In some embodiments, R⁴ or J is piperidinyl, piperazinyl, tetahydropyranyl, morpholinyl, or pyrrolidinyl, each of which is independently substituted with at least halo-. In some embodiments, R⁴ or J is piperidinyl substituted with halo-. In some embodiments, R⁴ or J is methylpiperidinyl substituted with halo-. In some embodiments, R⁴ or J is 3-fluoro-1-methylpiperidinyl. In some embodiments, R⁴ or J is 3-fluoro-1-(2-hydroxy-3-methoxypropyl)piperidinyl. In some embodiments, R⁴ or J is tetrahydropyranyl substituted with at least halo-.

In some embodiments, R⁴ is piperidinyl, piperazinyl, tetahydropyranyl, morpholinyl, or pyrrolidinyl, each of which is independently substituted with at least halo-. In some embodiments, R⁴ is piperidinyl substituted with halo-. In some embodiments, R⁴ is methylpiperidinyl substituted with halo-. In some embodiments, R⁴ is 3-fluoro-1-methylpiperidinyl. In some embodiments, R⁴ is 3-fluoro-1-(2-hydroxy-3-methoxypropyl)piperidinyl. In some embodiments, R⁴ is tetrahydropyranyl substituted with at least halo-.

In some embodiments, J is piperidinyl, piperazinyl, tetahydropyranyl, morpholinyl, or pyrrolidinyl, each of which is independently substituted with at least halo-. In some embodiments, J is piperidinyl substituted with halo-. In some embodiments, J is methylpiperidinyl substituted with halo-. In some embodiments, J is 3-fluoro-1-methylpiperidinyl. In some embodiments, J is 3-fluoro-1-(2-hydroxy-3-methoxypropyl)piperidinyl. In some embodiments, J is tetrahydropyranyl substituted with at least halo-.

In some embodiments, R⁴ or J is a ring that is:

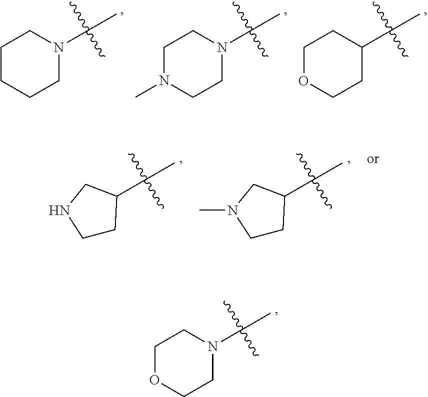

wherein the ring is substituted or unsubstituted. In some embodiments, the ring is substituted with halo-. In some embodiments, the ring is substituted with fluoro. In some embodiments, R³ is H, and R⁴ is a ring that is

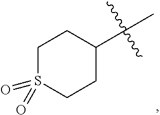 [SMILES: *C1CCS(=O)(=O)CC1]

wherein the ring is substituted or unsubstituted. In some embodiments, the ring is substituted with halo-. In some embodiments, the ring is substituted with fluoro. In some embodiments, R³ is H, and R⁴ is a ring that is

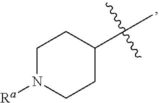

wherein the ring is substituted or unsubstituted. In some embodiments, Ra is alkylene. In some embodiments, Ra is methyl. In some embodiments, the ring is substituted with halo. In some embodiments, the ring is substituted with fluoro. In some embodiments, R³ is H, and R⁴ is a ring that is

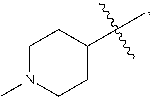 [SMILES: *C1CCN(C)CC1]

wherein the ring is substituted or unsubstituted. In some embodiments, the ring is substituted with halo. In some embodiments, the ring is substituted with fluoro. In some embodiments, R³ is H, and R⁴ is a ring that is

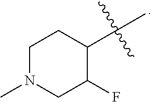 [SMILES: *C1CCN(C)CC1F]

In some embodiments, the R⁴ or J is substituted with one or more substituents selected from a hydroxyl group, sulfhydryl group, halogens, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamoyl group, carbamate group, amide group, urethane group, and ester group.

In some embodiments, R³ and R⁴ together with the nitrogen atom to which R³ and R⁴ are bound form a ring, wherein the ring is substituted or unsubstituted. In some embodiments, R³ and R⁴ together with the nitrogen atom to which R³ and R⁴ are bound form a substituted heterocycle. In some embodiments, R³ and R⁴ together with the nitrogen atom to which R³ and R⁴ are bound form a heterocycle substituted with a hydroxyl group, halogen, amino group, or alkyl group. In some embodiments, R³ and R⁴ together with the nitrogen atom to which R³ and R⁴ are bound form a heterocycle, wherein the heterocycle is substituted by a substituted or unsubstituted heterocycle.

In some embodiments, R³ and R⁴ together with the nitrogen atom to which R³ and R⁴ are bound form a ring of a following formula:

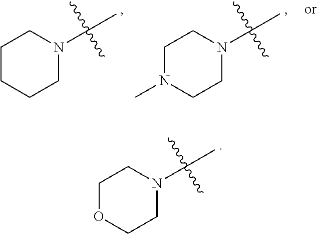

In some embodiments, the compound is of the formula:

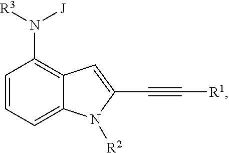

or a pharmaceutically-acceptable salt thereof, wherein J is a cyclic group that is substituted or unsubstituted; R¹ is alkyl, or alkenyl, each of which is unsubstituted or substituted, —C(O)R¹⁶, —C(O)OR¹⁶, or —C(O)NR¹⁶R¹⁷; R² is substituted or unsubstituted alkyl; and R³ is H.

In some embodiments, the compound is of the formula:

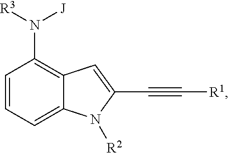

or a pharmaceutically-acceptable salt thereof,

wherein:
J is a cyclic group that is substituted or unsubstituted;
R¹ is alkyl, or alkenyl, each of which is unsubstituted or substituted, —C(O)R¹⁶, —C(O)OR¹⁶, or —C(O)NR¹⁶R¹⁷;
R¹⁶ and R¹⁷ are each independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R²¹ and R²² is independently alkyl, alkenyl, alkynyl, aryl, heteroaryl, or heterocyclyl, each of which is independently substituted or unsubstituted, or hydrogen; and
R² is substituted or unsubstituted alkyl; and
R³ is H.

In some embodiments, R² is substituted ethyl. In some embodiments, R² is trifluoroethyl. In some embodiments, R² is 2,2,2-trifluoroethyl.

In some embodiments, J is aryl, heteroaryl, or heterocyclyl, each of which is substituted or unsubstituted. In some embodiments, J is substituted heterocyclyl.

In some embodiments, R³ is H, J is piperidinyl, piperazinyl, tetahydropyranyl, morpholinyl, or pyrrolidinyl, each of which is independently substituted or unsubstituted.

In some embodiments, R¹ is alkyl substituted with NR¹⁶R¹⁷; and each R¹⁶ and R¹⁷ is independently alkyl, alkenyl, aryl, heteroaryl, heterocyclyl, each of which is independently substituted or unsubstituted; or hydrogen.

In some embodiments, the compound is of the formula:

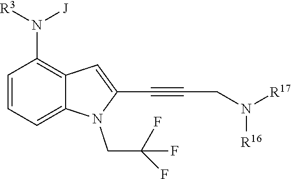

In some embodiments, R¹⁶ is hydrogen or alkyl. In some embodiments, R¹⁷ is aryl, heteroaryl, or heterocyclyl, each of which is independently substituted or unsubstituted. In some embodiments, R¹⁷ is substituted aryl. In some embodiments, R¹⁷ is phenyl substituted with a sulfoxide group, sulfonyl group, carboxyl group, amide group, amino group, alkyl, alkoxy, hydroxy, or heterocyclyl, each of which is independently substituted or unsubstituted, or halo or cyano.

In some embodiments, the compound is of the formula:

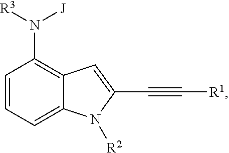

or a pharmaceutically-acceptable salt thereof, wherein:
J is C₃₋₉cycloalkyl, C₆₋₁₀aryl, C₁₋₉heteroaryl, or C₁₋₉heterocyclyl, each of which is independently unsubstituted or substituted with 1 to 7 R^{C};
R¹ is C₁₋₆alkyl, C₂₋₆alkenyl, each of which is independently unsubstituted or substituted with 1 to 7 R^{D}, or —(CH₂)_{w}C(O)R¹⁶, —(CH₂)_{w}C(O)OR¹⁶, —(CH₂)_{w}NR¹⁶R¹⁷, or —(CH₂)_{w}C(O)NR¹⁶R¹⁷, wherein w is 0, 1, 2, or 3;
R² is C₁₋₆alkyl that is independently unsubstituted or substituted with 1 to 7 R^{F};
each R¹⁶ and R¹⁷ is independently C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{F}, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R²¹ and R²² is independently C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with 1 to 7 R^{H};
each R^{C}, R^{D}, R^{F}, and R^{H} is independently selected from —CN, halogen, —C(O)R²³, —C(O)OR²³, —NR²³R²⁴, —OR²³, —C(O)NR²³R²⁴, —SR²³, —S(O)R²³, —SO₂R²³, —NR²³C(O)R²³, and —OC(O)R²³; or C₁₋₆alkyl, C₁₋₆heteroalkyl C₆₋₁₀aryl, and C₁₋₉heterocyclyl, C₁₋₆alkylene-C₆₋₁₀aryl, and C₁₋₆alkylene-C₁₋₉heterocyclyl, each of which is independently unsubstituted or substituted with —CN, halogen, methyl, ethyl, propyl, butyl, —C(O)R²³, —C(O)OR²³, —C(O)NR²³R²⁴, —OR²³, —SR²³, —S(O)R²³, —SO₂R²³, —NR²³R²⁴, —NR²³C(O)R²⁴, or —OC(O)R²³;
each R²³ and R²⁴ is independently hydrogen, or C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with —CN, halogen, C₁₋₆alkyl, or C₁₋₆haloalkyl.

In some embodiments, R² is ethyl substituted with 1 to 3 R^{F}. In some embodiments, R² is trifluoroethyl. In some embodiments, R² is 2,2,2-trifluoroethyl.

In some embodiments, J is C₆₋₁₀aryl, C₁₋₉heteroaryl, or C₁₋₉heterocyclyl, each of which is independently unsubstituted or substituted with 1 to 7 R^{C}. In some embodiments, C₁-9heterocyclyl that is unsubstituted or substituted with 1 to 7 R^{C}. In some embodiments, 6-membered heterocyclyl that is unsubstituted or substituted with 1 to 7 R^{C}.

In some embodiments, R³ is H, and J is piperidinyl, piperazinyl, tetahydropyranyl, morpholinyl, or pyrrolidinyl, each of which is independently unsubstituted or substituted with 1 to 7 R^{C}.

In some embodiments, R¹ is C₁₋₆alkyl substituted with NR¹⁶R¹⁷; and each R¹⁶ and R¹⁷ is independently C₁₋₆alkyl, C₂₋₆alkynyl, C₁₋₉heteroaryl, or C₁₋₉heterocyclyl, each of which is independently unsubstituted or substituted with 1 to 7 R^{D}; or hydrogen. In some embodiments, R¹ is —(CH₂)_{w}NR¹⁶R¹⁷, wherein w is 0, 1, 2, or 3. In some embodiments, R¹ is —CH₂NR¹⁶R¹⁷.

In some embodiments, the compound is of the formula:

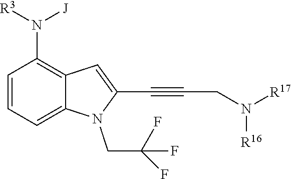

In some embodiments, R¹⁶ is hydrogen or C₁₋₆alkyl that is unsubstituted or substituted with 1 to 7 R^{F}. In some embodiments, R¹⁷ is C₆₋₁₀aryl, C₁₋₉heteroaryl, or C₁₋₉heterocyclyl each of which is independently unsubstituted or substituted with 1 to 7 R^{F}. In some embodiments, R¹⁷ is C₆₋₁₀aryl that is substituted with 1 to 7 R^{F}. In some embodiments, R¹⁷ is phenyl substituted with 1 to 5 R^{F}. In some embodiments, R¹⁷ is phenyl substituted 1 to 5 groups independently selected from halogen, cyano, —S(O)R²³, —SO₂R²³, —C(O)OR²³, —C(O)NR²³R²⁴, —NR²³C(O)R²⁴, —NR²³R²⁴, or —OR²³; or C₁₋₆alkyl or C₁₋₉heterocyclyl, each of which is independently unsubstituted or substituted with —CN, halogen, methyl, ethyl, propyl, butyl, —C(O)R²³, —C(O)OR²³, —C(O)NR²³R²⁴, —OR²³, —SR²³, —S(O)R²³, —SO₂R²³, —NR²³R²⁴, —NR²³C(O)R²⁴, or —OC(O)R²³. In some embodiments, R¹⁷ is phenyl substituted 1 to 5 groups independently selected from halogen, cyano, —S(O)R²³, —S(O)₂R²³, —C(O)OR²³, —C(O)NR²³R²⁴, —NR²³C(O)R²⁴, —NR²³R²⁴, or —OR²³.

In some embodiments, the compound is of the formula:

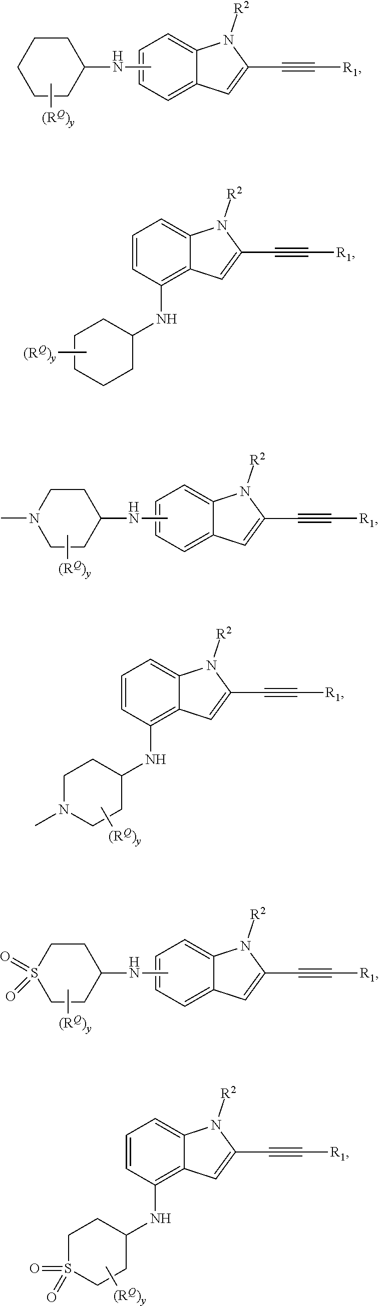

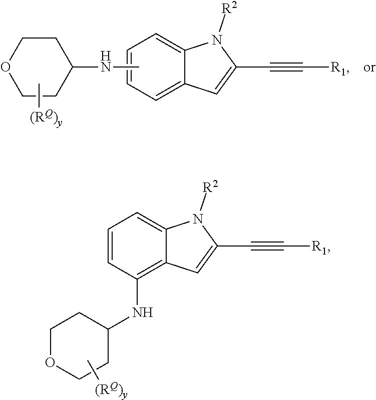

wherein:
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R^{Q} is independently alkyl, alkenyl, alkynyl, aryl, heteroaryl, or heterocyclyl, each of which is independently substituted or unsubstituted, or hydrogen or halogen;
y is 0, 1, 2, 3, or 4;
each R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, or aryl, each of which is independently substituted or unsubstituted; or —NR²¹R²², halo or hydrogen.

In some embodiments, R¹ is substituted C₁-C₃-alkyl. In some embodiments, R¹ is C₁-C₃-alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group. In some embodiments, R¹ is methyl substituted with NR¹⁶R¹⁷, wherein R¹⁶ is hydrogen, and R¹⁷ is substituted aryl. In some embodiments, R¹ is methyl substituted with NR¹⁶R¹⁷, wherein R¹⁶ is hydrogen, and R¹⁷ is substituted phenyl. In some embodiments, R¹ is methyl substituted with NR¹⁶R¹⁷, wherein R¹⁶ is hydrogen, and R¹⁷ is phenyl substituted with a sulfoxide group, a sulfonyl group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is a substituted amide group. In some embodiments, R¹⁷ is phenyl substituted with an amide group. In some embodiments, R¹⁷ is phenyl substituted with at least a methoxy group. In some embodiments, R¹⁷ is substituted with methoxy and sulfonamide. In some embodiments, R¹⁷ is substituted with methoxy and an amide group. In some embodiments, R¹⁷ is substituted with methoxy and sulfonyl. In some embodiments, R¹⁷ is phenyl substituted with —C(O)NH(C₁-C₆alkyl), —S(O)₂(C₁-C₆alkyl), or C₁-C₆alkoxy. In some embodiments, R¹⁷ is phenyl substituted with —C(O)NH(C₁-C₆alkyl) and C₁-C₆alkoxy. In some embodiments, R¹⁷ is phenyl substituted with —S(O)₂(C₁-C₆alkyl) and C₁-C₆alkoxy. In some embodiments, R¹⁷ is phenyl substituted with —C(O)NH(C₁-C₆hydroxyalkyl) and C₁-C₆alkoxy. In some embodiments, R¹⁷ is phenyl substituted with —C(O)NH(C₁-C₆alkoxy) and C₁-C₆alkoxy.

In some embodiments, R² is hydrogen or alkyl. In some embodiments, R² is substituted C₁-C₅-alkylene. In some embodiments, R2 is trifluoroethyl. In some embodiments, R¹³ is alkyl, alkenyl, hydrogen, or halogen. In some embodiments, R2 is alkyl, and R¹³ is alkyl. In some embodiments, R2 is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen.

In some embodiments, the compound is of the formula:

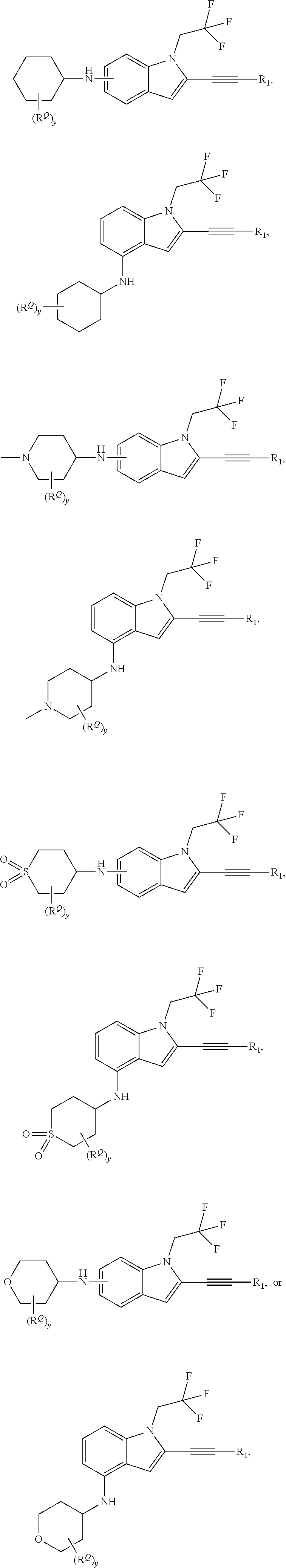

or a pharmaceutically-acceptable salt thereof, wherein the variables are as defined above.

In some embodiments, each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹ hydrogen, or halogen. In some embodiments, each R^{Q} is —NR²¹R²² or halogen. In some embodiments, each R^{Q} is NH₂ or halogen.

In some embodiments, y is 1. In some embodiments, y is 2. In some embodiments, y is 3. In some embodiments, y is 4.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, —NR²¹R²², or aryl, each of which is independently substituted or unsubstituted; halo or hydrogen.

In some embodiments, R¹ is substituted alkyl. In some embodiments, R¹ is substituted C₁-C₃-alkyl. In some embodiments, R¹ is alkyl substituted with NR¹⁶R¹⁷. In some embodiments, R¹ is C₁-C₃-alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, R¹⁶ is alkyl, alkenyl, aryl, heteroaryl, heterocyclyl, or hydrogen, and R¹⁷ is aryl, heteroaryl, or heterocyclyl. In some embodiments, R¹⁶ is hydrogen, and R¹⁷ is phenyl, indolyl, piperidinyl, imidazolyl, thiazolyl, morpholinyl, pyrrolyl, or pyridinyl, each of which is substituted or unsubstituted.

In some embodiments, the compound is of the formula:

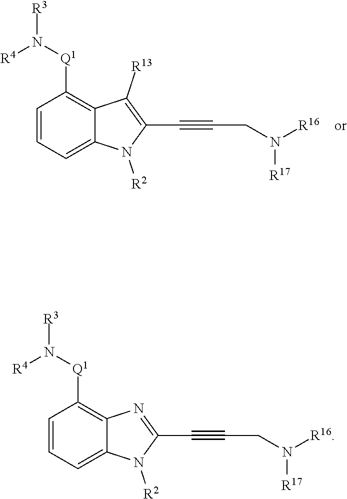

In some embodiments, the compound is of the formula:

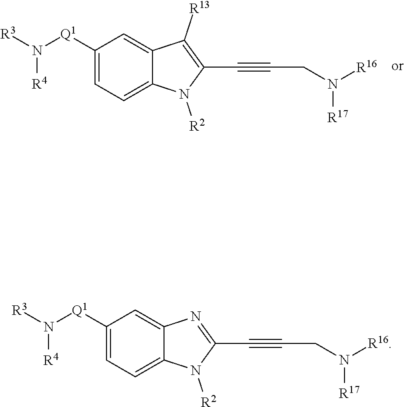

In some embodiments, the compound is of the formula:

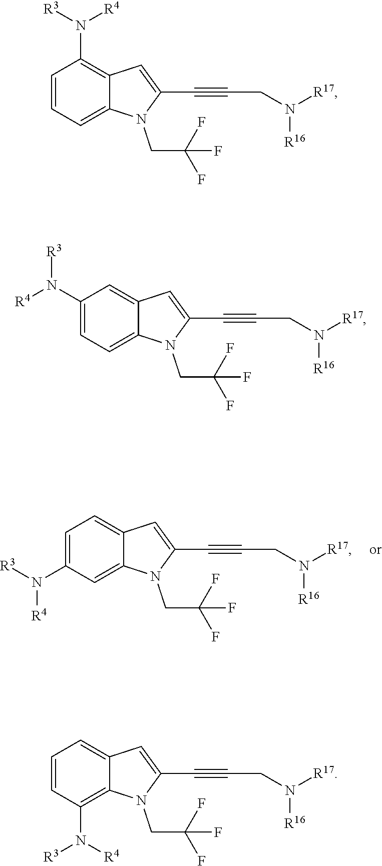

In some embodiments, each R¹⁶ and R¹⁷ is independently alkyl, alkenyl, aryl, heteroaryl, heterocyclyl, or hydrogen. In some embodiments, R¹⁶ is aryl, and R¹⁷ is alkyl. In some embodiments, R¹⁶ is aryl, and R¹⁷ is hydrogen. In some embodiments, R¹⁶ is heteroaryl, and R¹⁷ is alkyl. In some embodiments, R¹⁶ is heteroaryl, and R¹⁷ is hydrogen. In some embodiments, R¹⁶ is substituted heteroaryl, and R¹⁷ is hydrogen. In some embodiments, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁶ is hydrogen. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano. In some embodiments, R¹⁶ is alkyl, alkenyl, aryl, heteroaryl, heterocyclyl, or hydrogen, and R¹⁷ is aryl, heteroaryl, or heterocyclyl. In some embodiments, R¹⁶ is hydrogen, and R¹⁷ is phenyl, indolyl, piperidinyl, imidazolyl, thiazolyl, morpholinyl, pyrrolyl, or pyridinyl, each of which is substituted or unsubstituted. In some embodiments, R¹⁶ is hydrogen, and R¹⁷ is substituted phenyl. In some embodiments, R¹⁶ is hydrogen, and R¹⁷ is phenyl substituted with a sulfoxide group, sulfonyl group, carboxyl group, amide group, amino group, alkyl, alkoxy, hydroxy, halo, cyano, or heterocyclyl, each of which is independently substituted or unsubstituted. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is a substituted amide group. In some embodiments, R¹⁷ is substituted with methoxy and sulfonamide.

In some embodiments, R¹⁷ is phenyl substituted with 1, 2, 3, or 4 R^{Z}, wherein each R^{Z} is independently selected from alkyl, alkenyl, alkynyl, aryl, heteroaryl, heterocyclyl, halo, —CN, —NR²¹R²², —OR²¹, —C(O)R²², —P(O)R²¹R²², —C(O)OR²¹, —C(O)NR²¹R²², —S(O)R²¹, —SO₂R²², —NS(O)R²¹, —NS(O)₂R²¹, —S(O)NR²¹R²², and —S(O)₂NR²¹R²².

In some embodiments, R¹⁷ is phenyl substituted with 1, 2, 3, or 4 R^{Z}, wherein each R^{Z} is independently selected from C₁₋₆alkyl, C₂₋₆alkenyl, C₂₋₆alkynyl, C₆₋₁₀aryl, C₁₋₉heteroaryl, C₁₋₉heterocyclyl, halo, —CN, —NR²¹R²², —OR²¹, —C(O)R²¹, —P(O)R²¹R²², —C(O)OR²¹, —C(O)NR²¹R²², —S(O)R²¹, —SO₂R²¹, —NS(O)R²¹, —NS(O)₂R²¹, —S(O)NR²¹R²², and —S(O)₂NR²¹R²².

In some embodiments, each R³ and R⁴ is independently unsubstituted or substituted alkyl. In some embodiments, R³ is hydrogen and R⁴ is —C(O)R¹⁹, —C(O)OR¹⁹, alkyl, alkenyl, alkynyl, aryl, heteroaryl, or heterocyclyl, each of which is independently substituted or unsubstituted. In some embodiments, R³ is hydrogen, and R⁴ is alkyl, aryl, heteroaryl, or heterocyclyl, each of which is independently substituted or unsubstituted. In some embodiments, R³ is H, and R⁴ is substituted heterocyclyl. In some embodiments, R³ is H, and R⁴ is substituted or unsubstituted C₄-C₆-heterocyclyl. In some embodiments, R³ is H, and R⁴ is substituted alkyl. In some embodiments, R³ is H, and R⁴ is substituted C₁-C₆-alkyl. In some embodiments, R³ is H, and R⁴ is substituted or unsubstituted cycloalkyl. In some embodiments, R³ is H, and R⁴ is substituted or unsubstituted C₄-C₆-cycloalkyl. In some embodiments, R³ is H, and R⁴ is C₄-C₆-cycloalkyl substituted with an amino group.

In some embodiments, each R³ and R⁴ is independently:
C₁₋₆alkyl, C₁₋₆heteroalkyl, C₂₋₆alkenyl, C₂₋₆alkynyl, C₃₋₉cycloalkyl, C₆₋₁₀aryl, C₁₋₉heteroaryl, or C₁₋₉heterocyclyl, each of which is independently unsubstituted or substituted with 1 to 7 RX; or
H, —C(O)R²⁴, —C(O)OR²⁴, —C(O)NR²⁴R²⁵, —SOR²⁴, or —SO₂R²⁴, or
R³ and R⁴ together with the nitrogen atom to which R³ and R⁴ are bound form C₁₋₉heterocyclyl, that is unsubstituted or substituted with 1 to 7 R^{X};

wherein
each R^{X} is independently selected from —CN, halogen, —C(O)R²⁴, —C(O)OR²⁴, —NR²⁴R²⁵, —OR²⁴, —C(O)NR²⁴R²⁵, —SR²⁴, —S(O)R²⁴, —SO₂R²⁴, —NR²⁴C(O)R²⁵, and —OC(O)R²⁴; or C₁₋₆alkyl, C₁₋₆heteroalkyl C₆₋₁₀aryl, and C₁₋₉heterocyclyl, C₁₋₆alkylene-C₆₋₁₀aryl, and C₁₋₆alkylene-C₁₋₉heterocyclyl, each of which is independently unsubstituted or substituted with —CN, halogen, methyl, ethyl, propyl, butyl, —C(O)R²⁸, —C(O)OR²⁸, —C(O)NR²⁸R²⁹, —OR²⁸, —SR²⁸, —S(O)R²⁸, —SO₂R²⁸, —NR²⁸R²⁹, —NR²⁸C(O)R²⁹, or —OC(O)R²⁸;
each R²⁴ and R²⁵ is independently C₁₋₆alkyl, C₂₋₆alkenyl, C₂₋₆alkynyl, C₆₋₁₀aryl, C₁₋₉heteroaryl, or C₁₋₉heterocyclyl, each of which is unsubstituted or substituted with halogen, methyl or ethyl; or H, —C(O)R²⁸, —C(O)OR²⁸, —C(O)NR²⁸R²⁹, —OR²⁸, —SR²⁸, —NR²⁸R²⁹, —NR²⁸C(O)R²⁹, or —OC(O)R²⁸; or R²⁴ and R²⁵ together with the nitrogen atom to which R²⁴ and R²⁵ are bound form a 5- or 6-membered ring, wherein the ring is unsubstituted or substituted with halogen, methyl, or ethyl; and
each R²⁶, R²⁷, R²⁸, and R²⁹ is independently C₁₋₆alkyl, C₂₋₆alkenyl, C₂₋₆alkynyl, C₆₋₁₀aryl, C₁₋₉heteroaryl, or C₁₋₉heterocyclyl, each of which is independently unsubstituted or substituted with halogen, cyano, hydroxy, amino, methyl, ethyl, propyl, or butyl; or hydrogen.

In some embodiments, R³ is C₃₋₉cycloalkyl, C₁₋₉heterocyclylalkyl, or C₁₋₆heteroalkyl, each of which is independently unsubstituted or substituted with 1 to 7 R^{X}. In some embodiments, R³ is C₁₋₉heterocyclylalkyl that is unsubstituted or substituted with 1 to 7 R^{X}. In some embodiments, R³ is a 6-membered heterocyclylalkyl that is unsubstituted or substituted with 1 to 7 R^{X}.

In some embodiments, each R^{X} is independently selected from —CN, halogen, —NR²⁴R²⁵, —C(O)R²⁵, —NR²⁵C(O)R²⁶, and —OR²⁴; or C₁₋₆alkyl, C₁₋₉heterocyclyl, and C₁₋₆heteroalkyl, each of which is independently unsubstituted or substituted with halogen, —CN, —C(O)NR²⁸R²⁹, —C(O)OR²⁸, —SO₂R²⁸, methyl, ethyl, or propyl.

In some embodiments, the compound is of the formula:

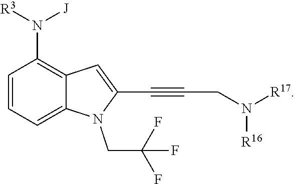

In some embodiments, each R³ and J is independently unsubstituted or substituted alkyl. In some embodiments, R³ is hydrogen and J is —C(O)R¹⁹, —C(O)OR¹⁹, alkyl, alkenyl, alkynyl, aryl, heteroaryl, or heterocyclyl, each of which is independently substituted or unsubstituted. In some embodiments, R³ is hydrogen, and J is alkyl, aryl, heteroaryl, or heterocyclyl, each of which is independently substituted or unsubstituted. In some embodiments, R³ is H, and J is substituted heterocyclyl. In some embodiments, R³ is H, and J is substituted or unsubstituted C₄-C₆-heterocyclyl. In some embodiments, R³ is H, and J is substituted alkyl. In some embodiments, R³ is H, and J is substituted C₁-C₆-alkyl. In some embodiments, R³ is H, and J is substituted or unsubstituted cycloalkyl. In some embodiments, R³ is H, and J is substituted or unsubstituted C₄-C₆-cycloalkyl. In some embodiments, R³ is H, and J is C₄-C₆-cycloalkyl substituted with an amino group.

In some embodiments, the compound is of the formula:

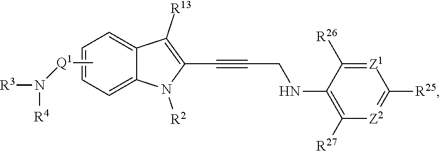

wherein:
Q¹ is alkylene, alkenylene, or alkynylene, each of which is independently substituted or unsubstituted, or C═O, C═S, C═CR¹⁴R¹⁵, C═NR¹⁴, or a bond;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ and R⁴ is independently alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each Z¹ and Z² is independently CR²⁸, CR²⁹, or N;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen;
each R²³ and R²⁴ is independently alkyl, alkenyl, alkynyl, aryl, heteroaryl, or heterocyclyl, each of which is independently substituted or unsubstituted, or hydrogen; and
each R²⁵, R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, Z¹ is N. In some embodiments, Z¹ and Z² are N. In some embodiments, each R²⁵ and R²⁶ is independently a halogen. In some embodiments, R²⁵ is

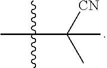 [SMILES: *C(C)(C)C#N]

In some embodiments, R²⁵ is a substituted sulfone group. In some embodiments, R²⁵ is a sulfone group substituted with alkyl. In some embodiments, R²⁵ is a methanesulfonyl group. In some embodiments, R²⁵ is a sulfone group substituted with an amino group. In some embodiments, R²⁵ is a sulfonamide. In some embodiments, R²⁵ is a carboxy group. In some embodiments, R²⁵ is a methoxycarbonyl group.

In some embodiments, the compound is of the formula:

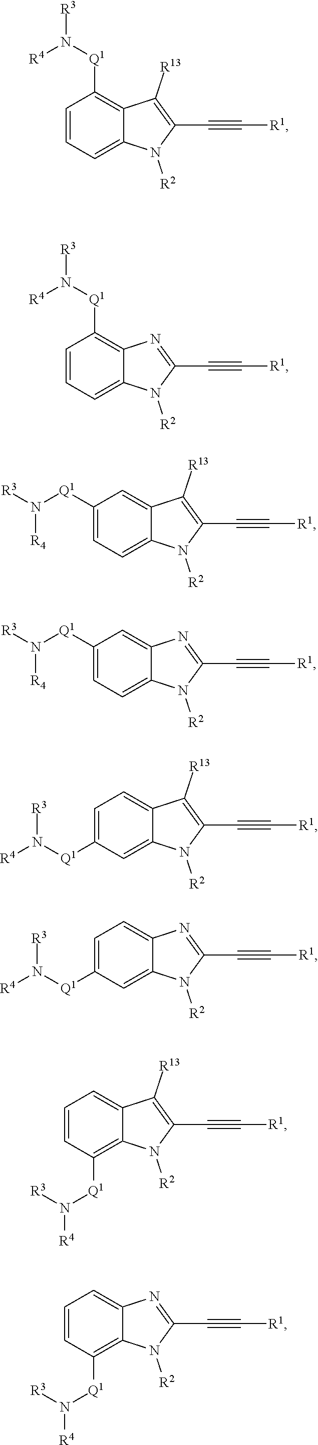

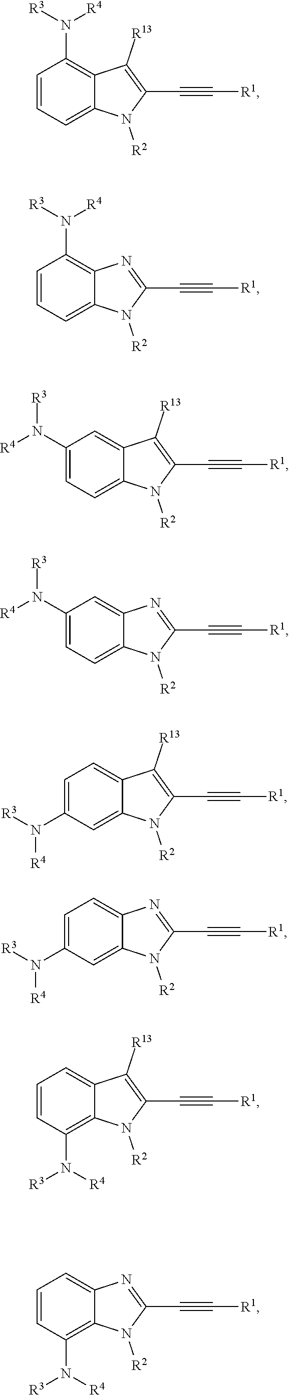

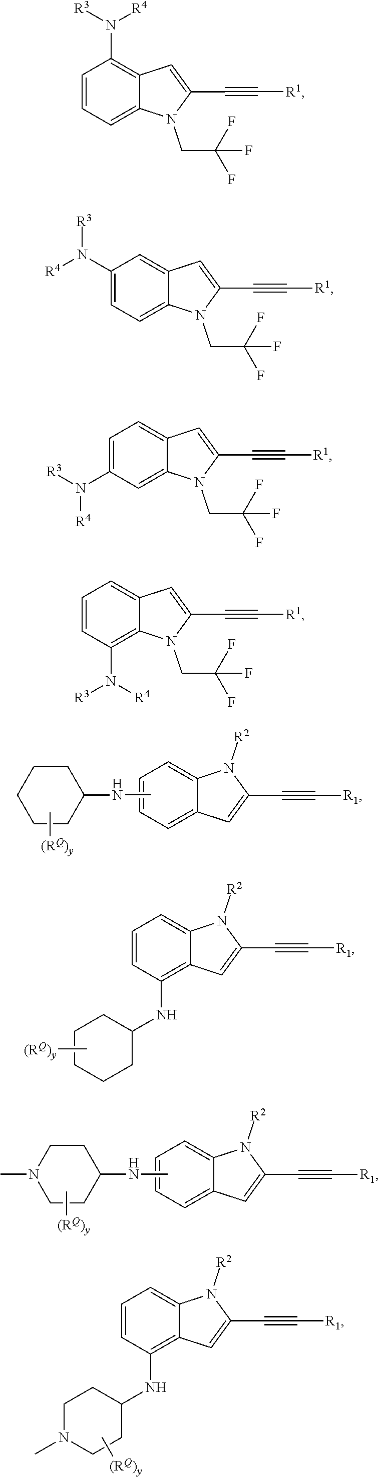

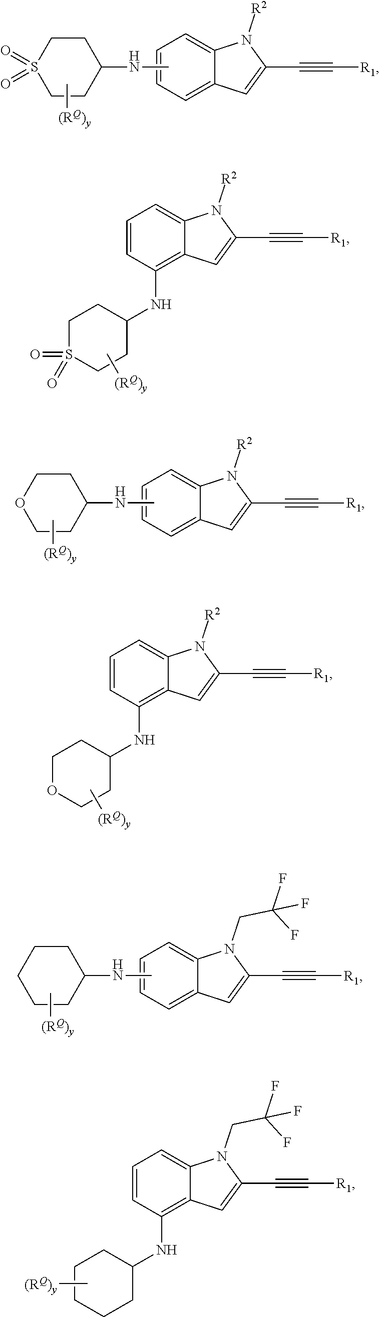

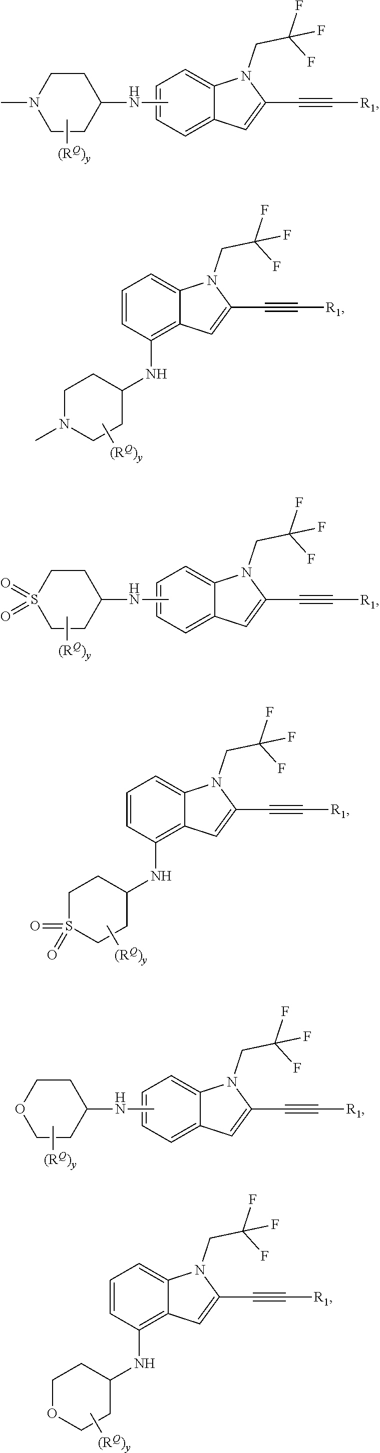

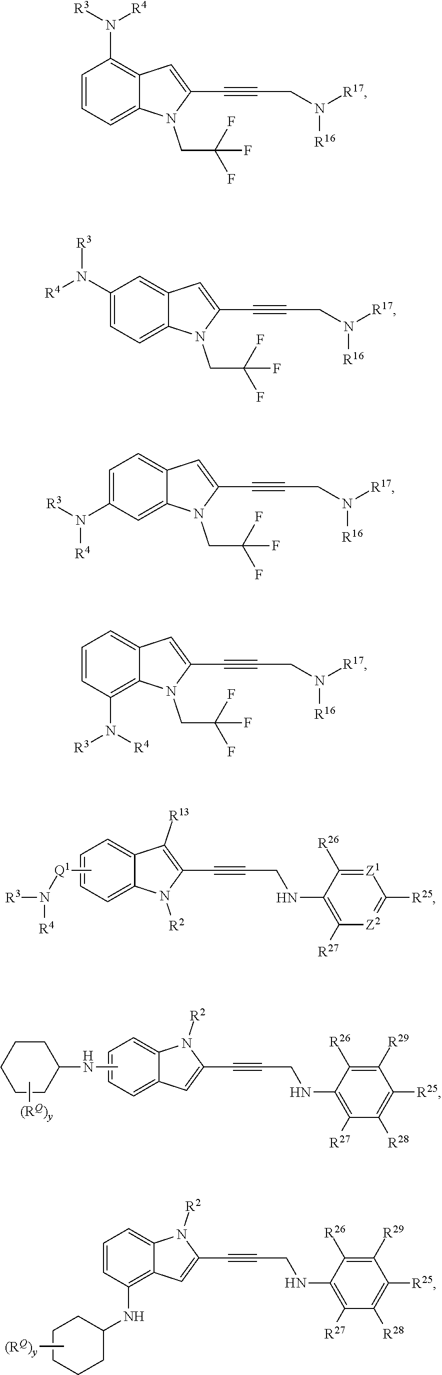

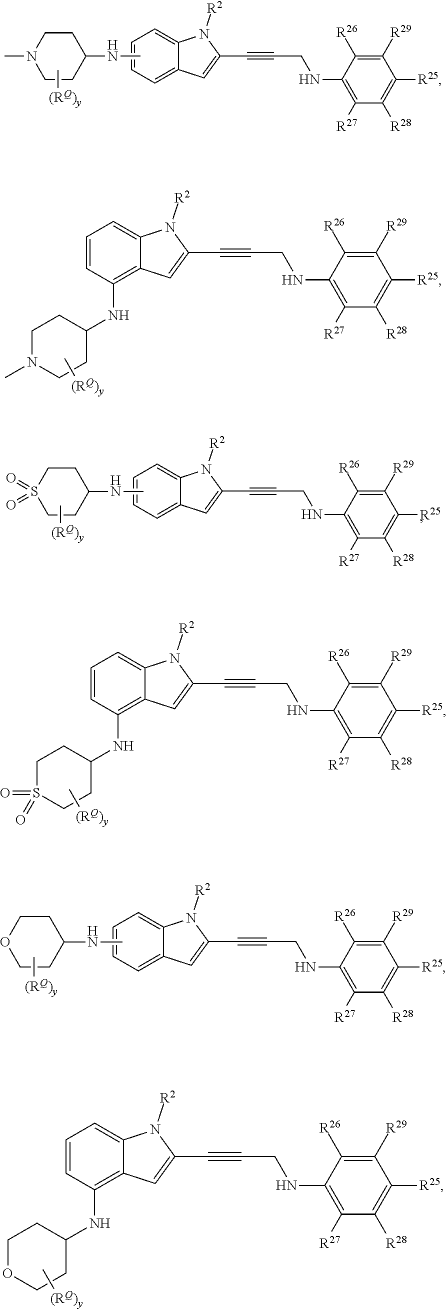

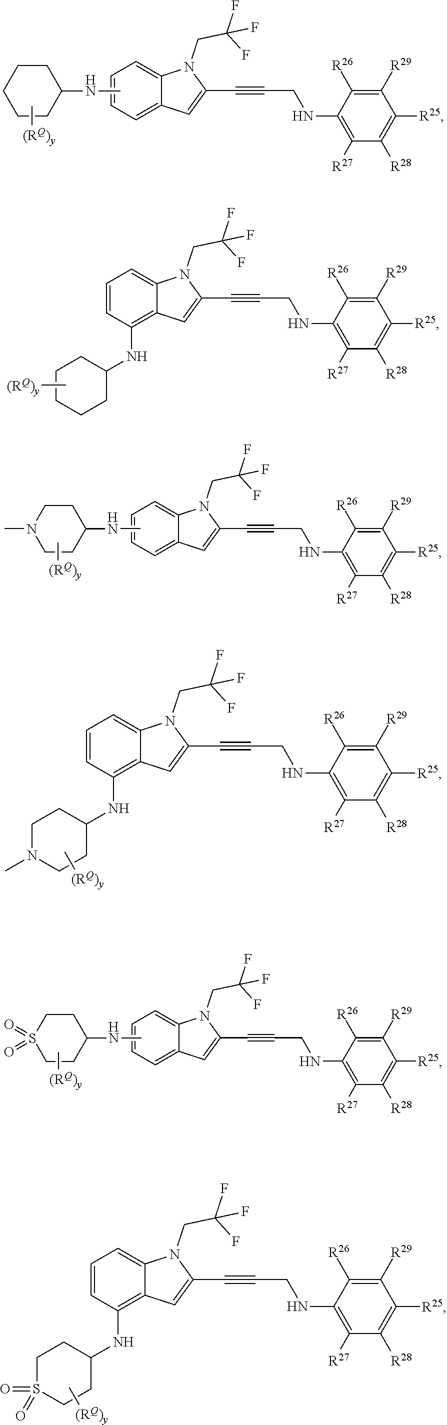

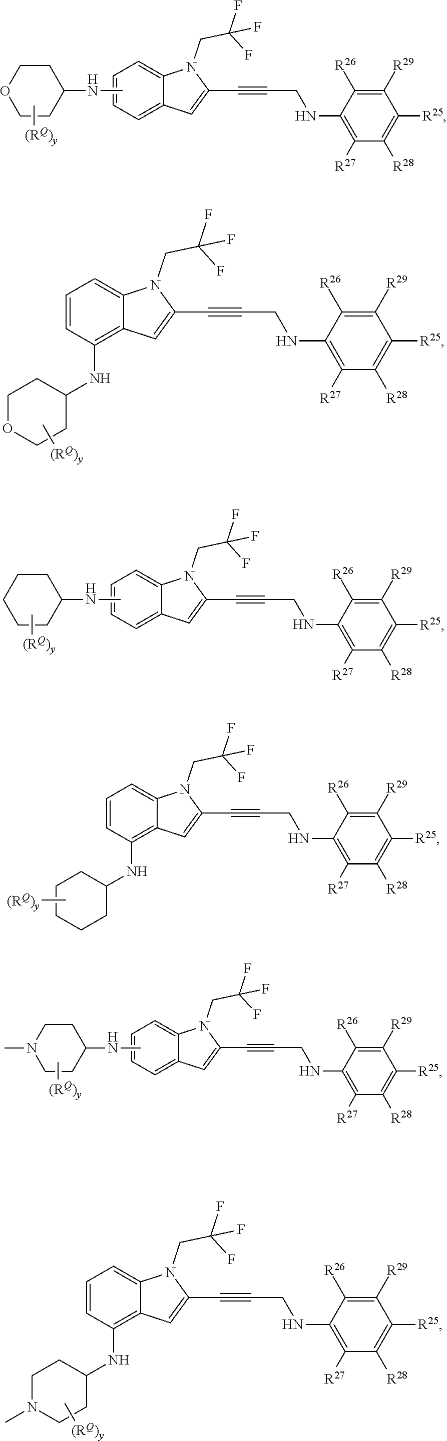

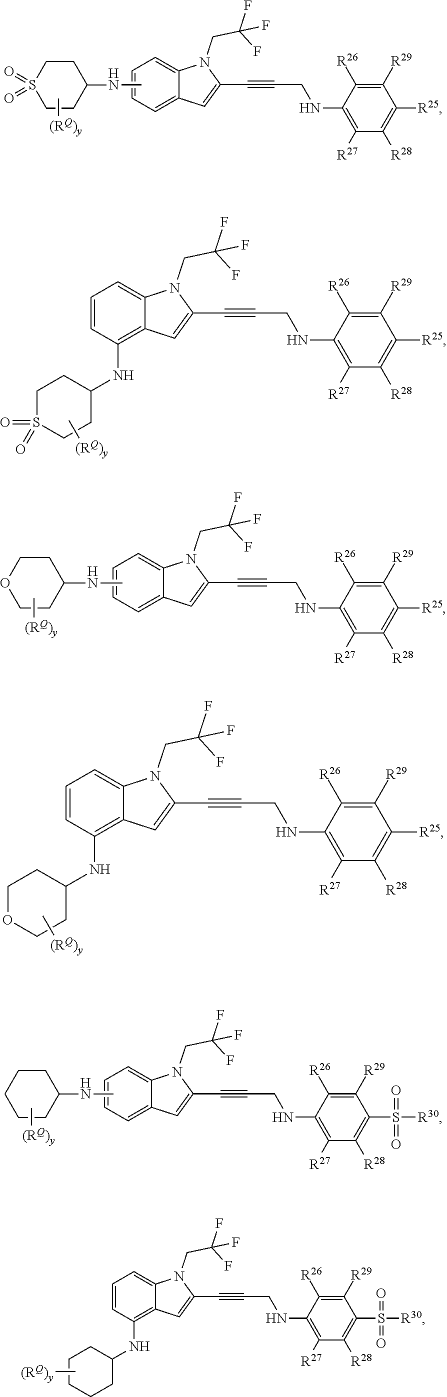

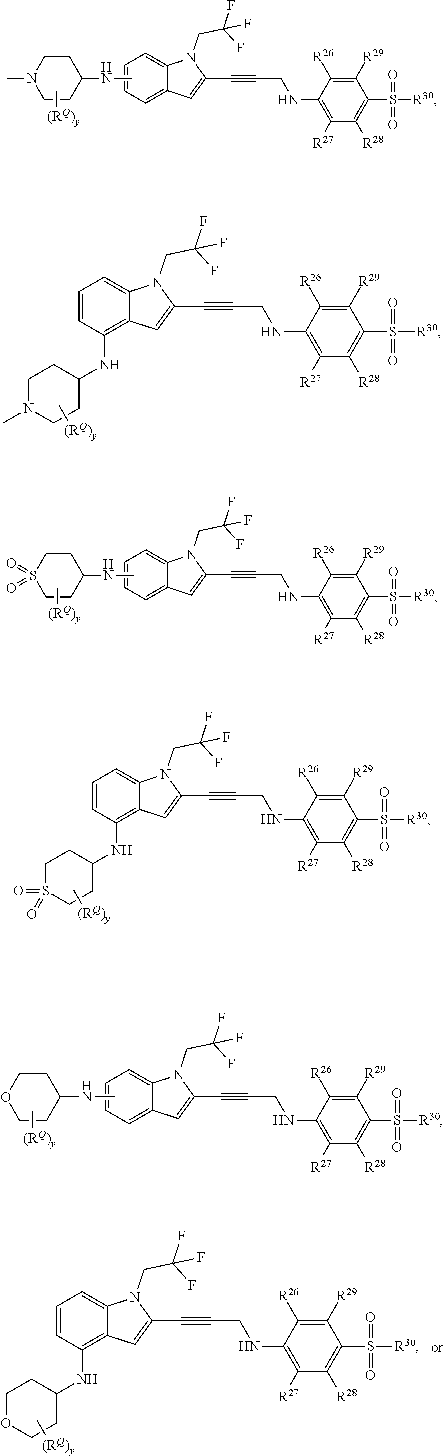

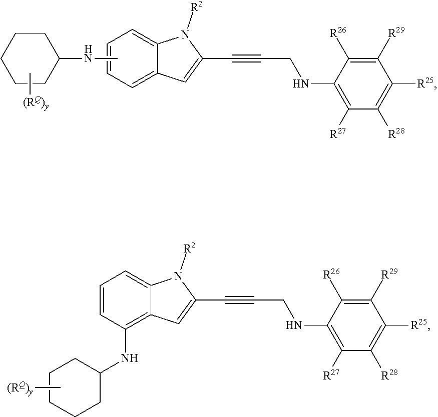

wherein:
R² is alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, cyano, or halogen;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen; and
each R²⁵, R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, the compound is of the formula:

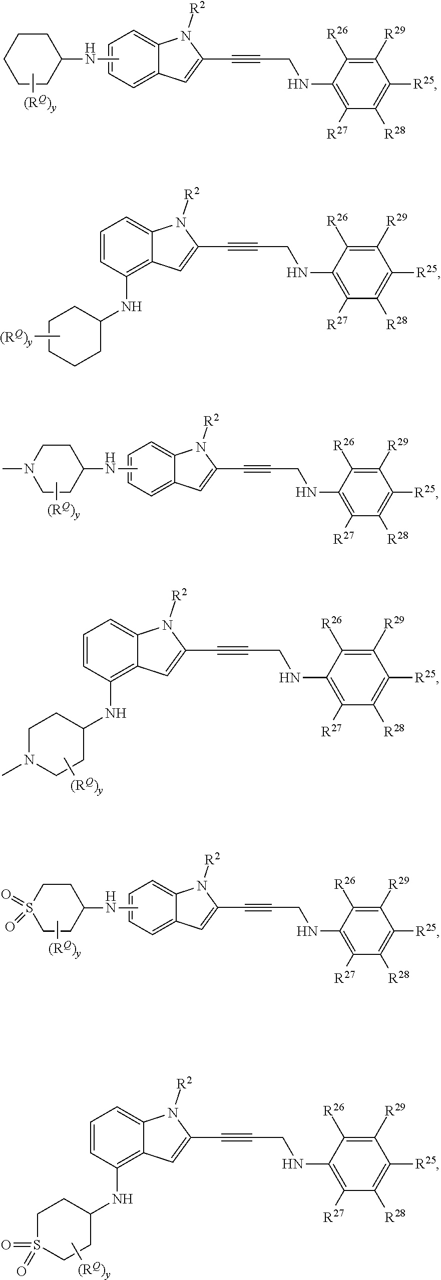

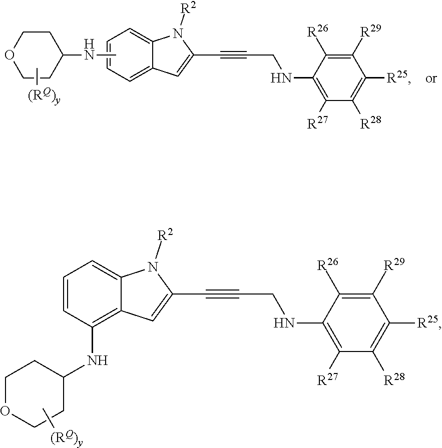

wherein:
R² is alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, cyano, or halogen;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen; and
each R²⁵, R²⁶, R²⁷, R²⁸, and R²⁹ is independently hydrogen or a substituent selected from a hydroxyl group, sulfydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, the compound is of the formula:

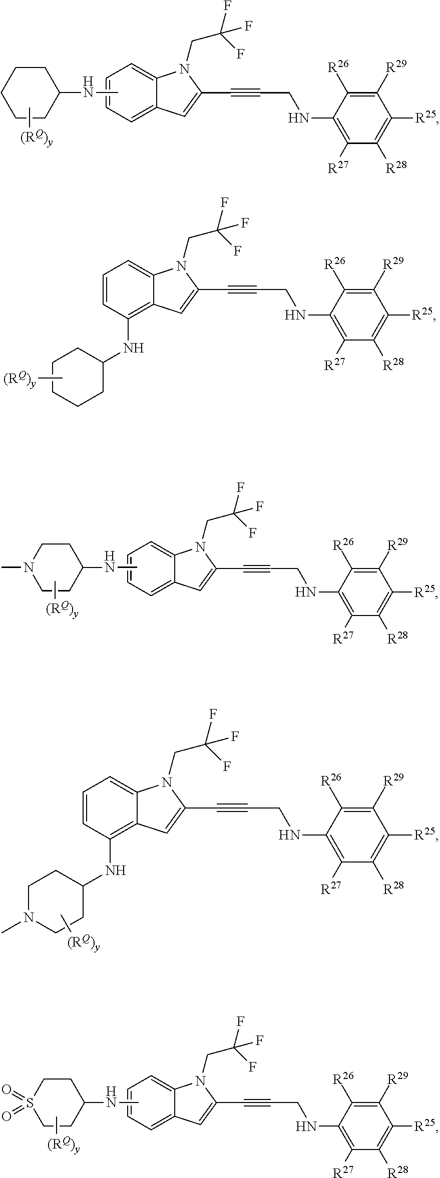

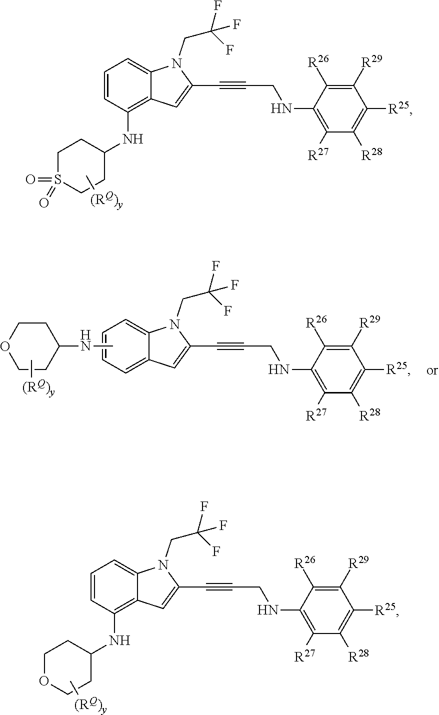

In some embodiments R²⁵ is a substituted sulfone group. In some embodiments, R²⁵ is a sulfone group substituted with alkyl. In some embodiments, R²⁵ is a methanesulfonyl group. In some embodiments, R²⁵ is a sulfone group substituted with an amino group. In some embodiments, R²⁵ is a sulfonamide. In some embodiments, R²⁵ is a carboxy group. In some embodiments, R²⁵ is a methoxycarbonyl group.

In some embodiments, the compound is of the formula:

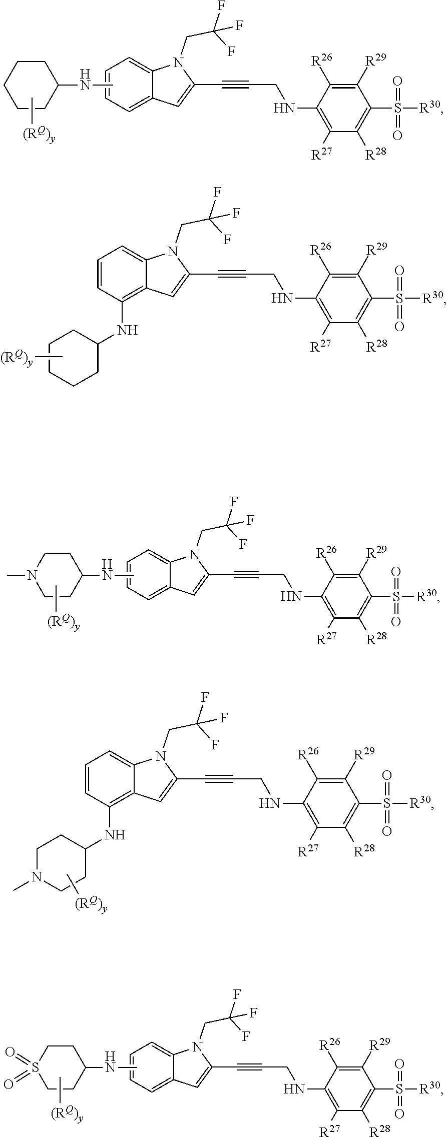

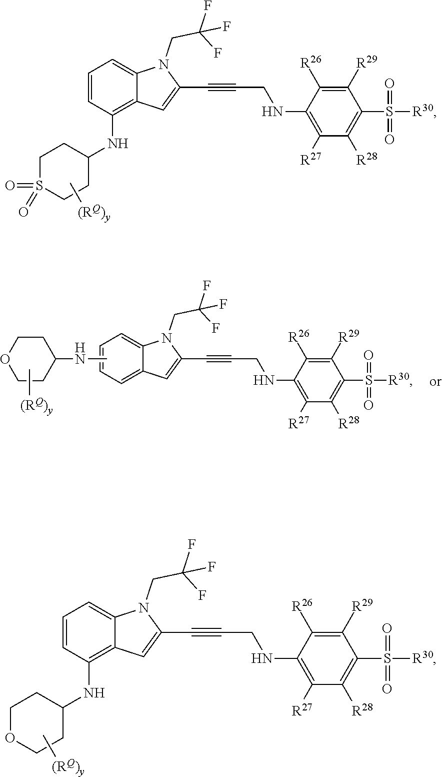

wherein:
each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, cyano, or halogen;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen;
each R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group; and
R³⁰ is alkyl or an amino group, each of which is substituted or unsubstituted,
   or a pharmaceutically-acceptable salt thereof.

In some embodiments, R³⁰ is methyl. In some embodiments, R³⁰ is NH₂. In some embodiments, R³⁰ is NHMe. In some embodiments, R³⁰ is NMe₂.

In some embodiments, the compound is of the formula:

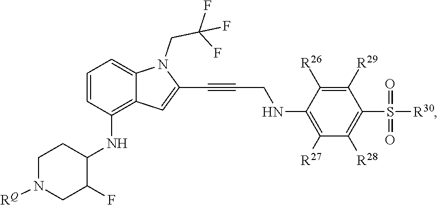

wherein R³⁰ is alkyl or an amino group, each of which is unsubstituted or substituted. In some embodiments, R³⁰ is methyl.

In some embodiments, the compound that increases anti-tumor activity of the mutant p53 protein is

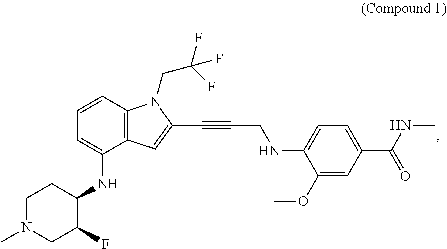

or a pharmaceutically-acceptable salt thereof.

Non-limiting examples of compounds of the current disclosure include the following:

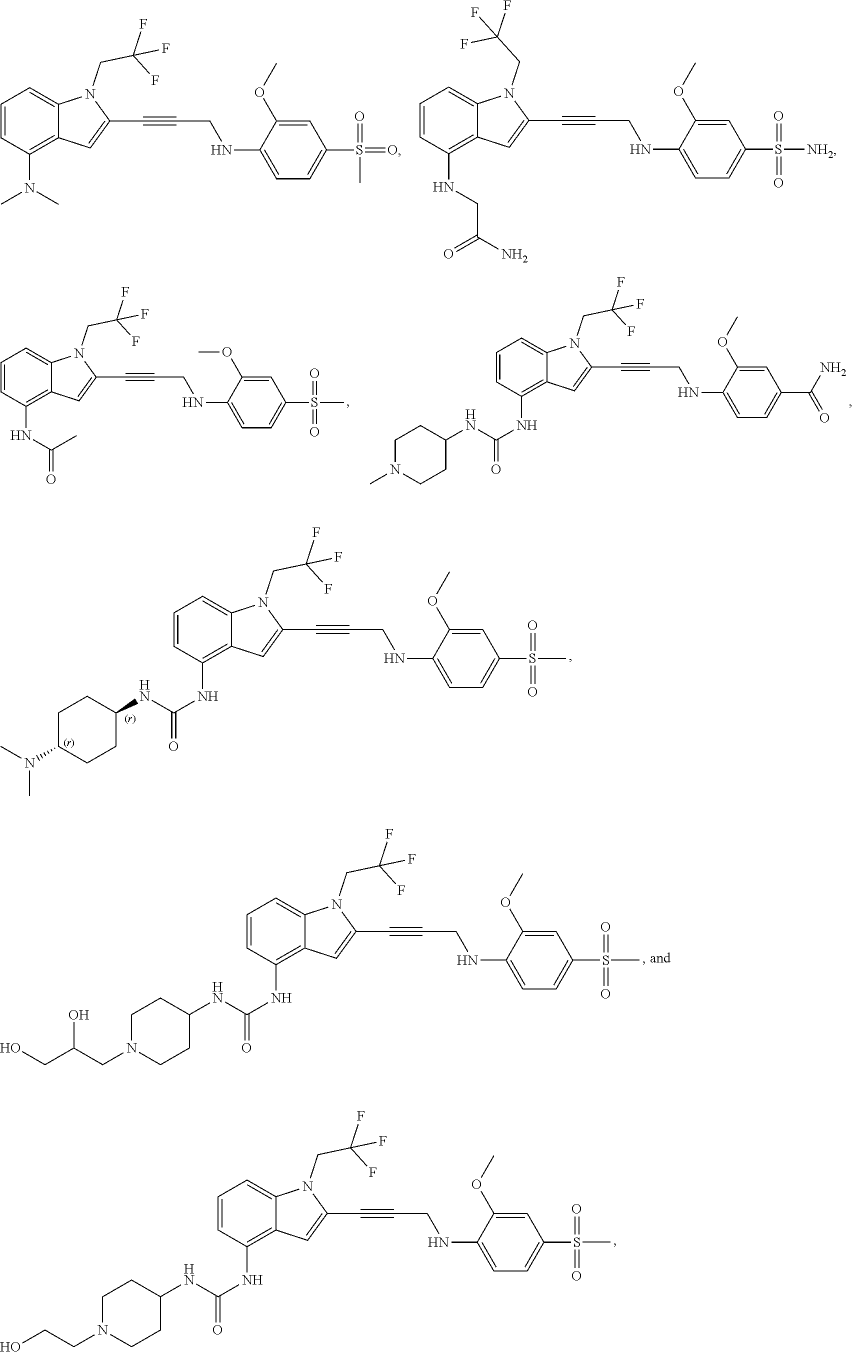

or a pharmaceutically-acceptable salt thereof.

Non-limiting examples of compounds of the current disclosure include the following:

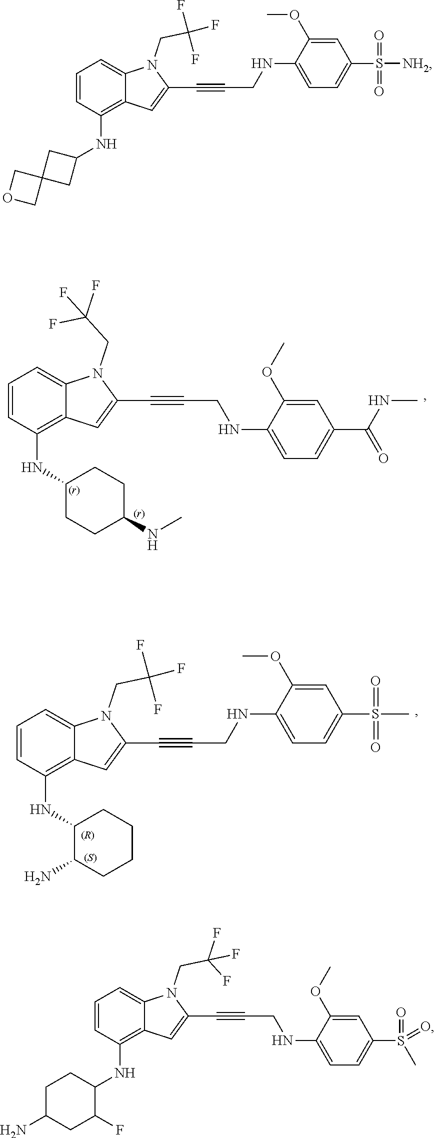

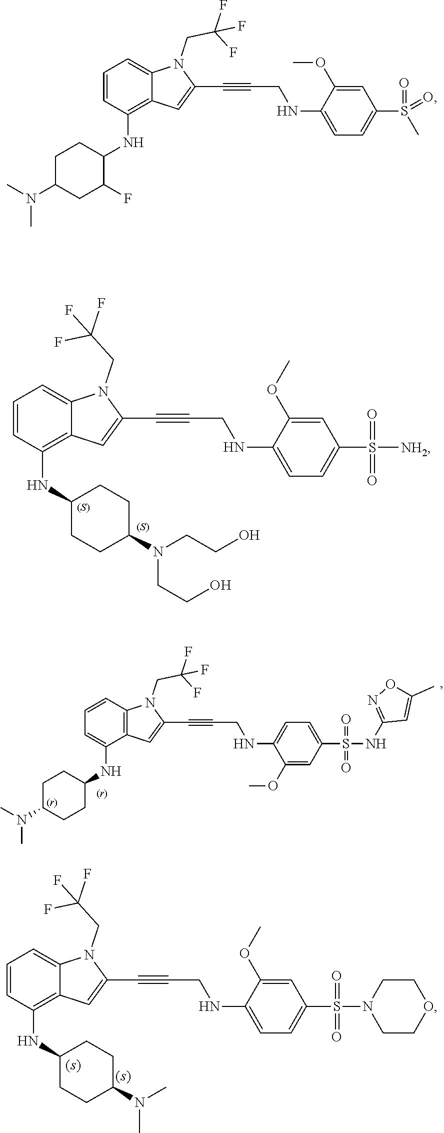

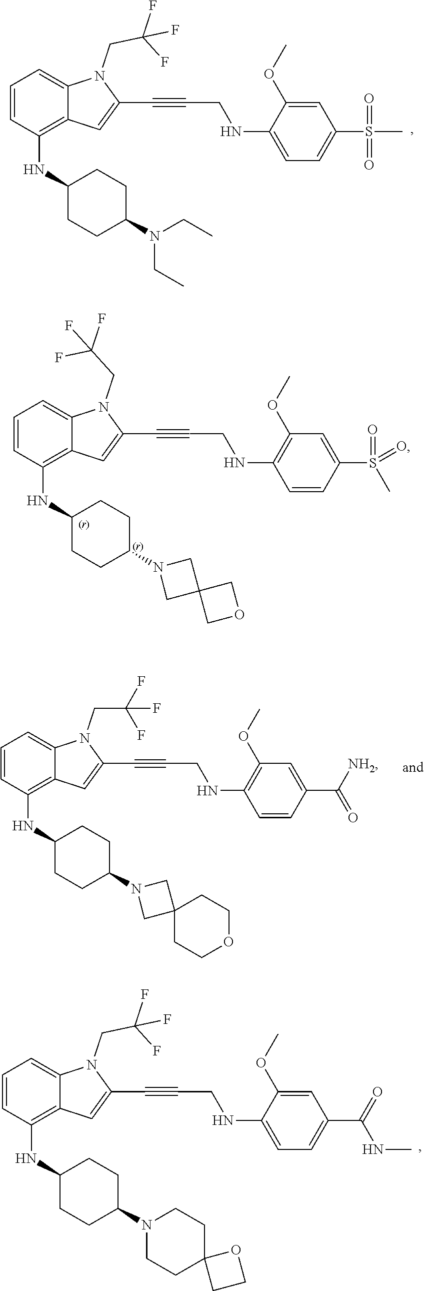

or a pharmaceutically-acceptable salt thereof.

Non-limiting examples of compounds of the current disclosure include the following:

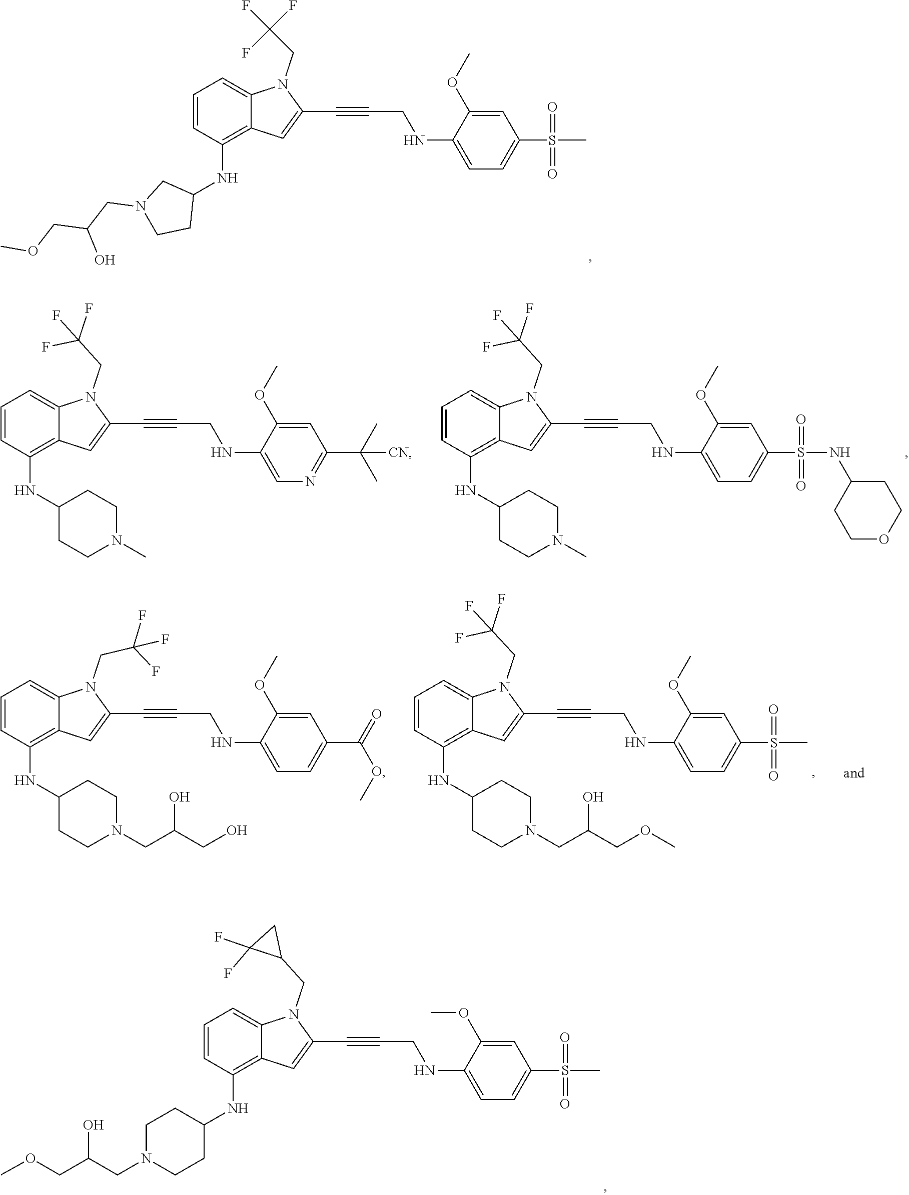

or a pharmaceutically-acceptable salt thereof.

Non-limiting examples of compounds of the current disclosure include the following:

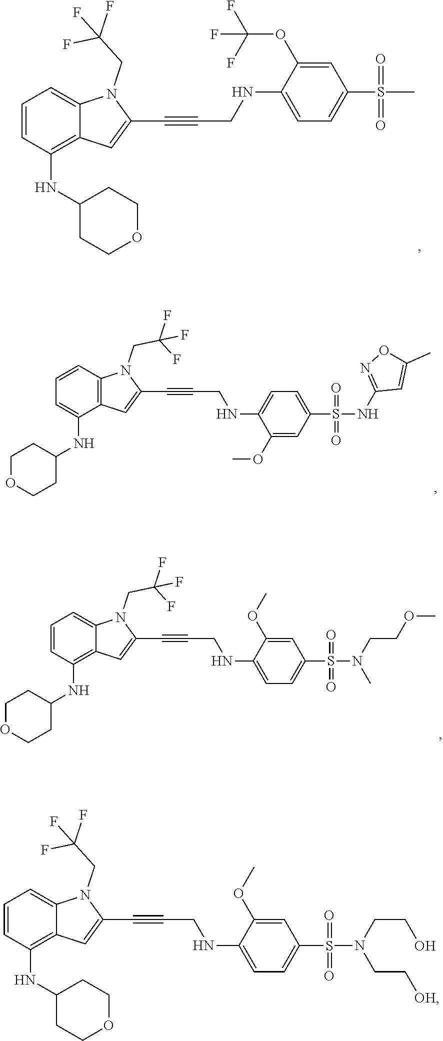 [SMILES: CS(=O)(=O)c1ccc(NCC#Cc2cc3c(NC4CCOCC4)cccc3n2CC(F)(F)F)c(OC(F)(F)F)c1 ; COc1cc(S(=O)(=O)Nc2cc(C)on2)ccc1NCC#Cc1cc2c(NC3CCOCC3)cccc2n1CC(F)(F)F ; COCCN(C)S(=O)(=O)c1ccc(NCC#Cc2cc3c(NC4CCOCC4)cccc3n2CC(F)(F)F)c(OC)c1 ; COc1cc(S(=O)(=O)N(CCO)CCO)ccc1NCC#Cc1cc2c(NC3CCOCC3)cccc2n1CC(F)(F)F]

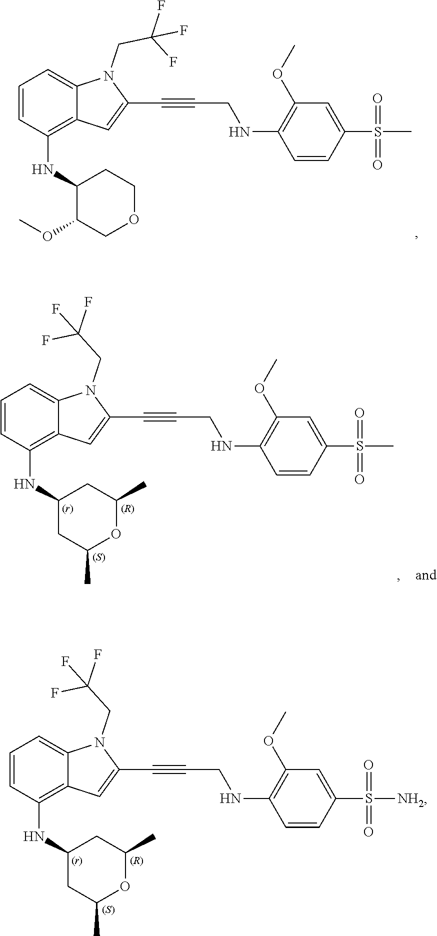

or a pharmaceutically-acceptable salt thereof.

Non-limiting examples of compounds of the current disclosure include the following:

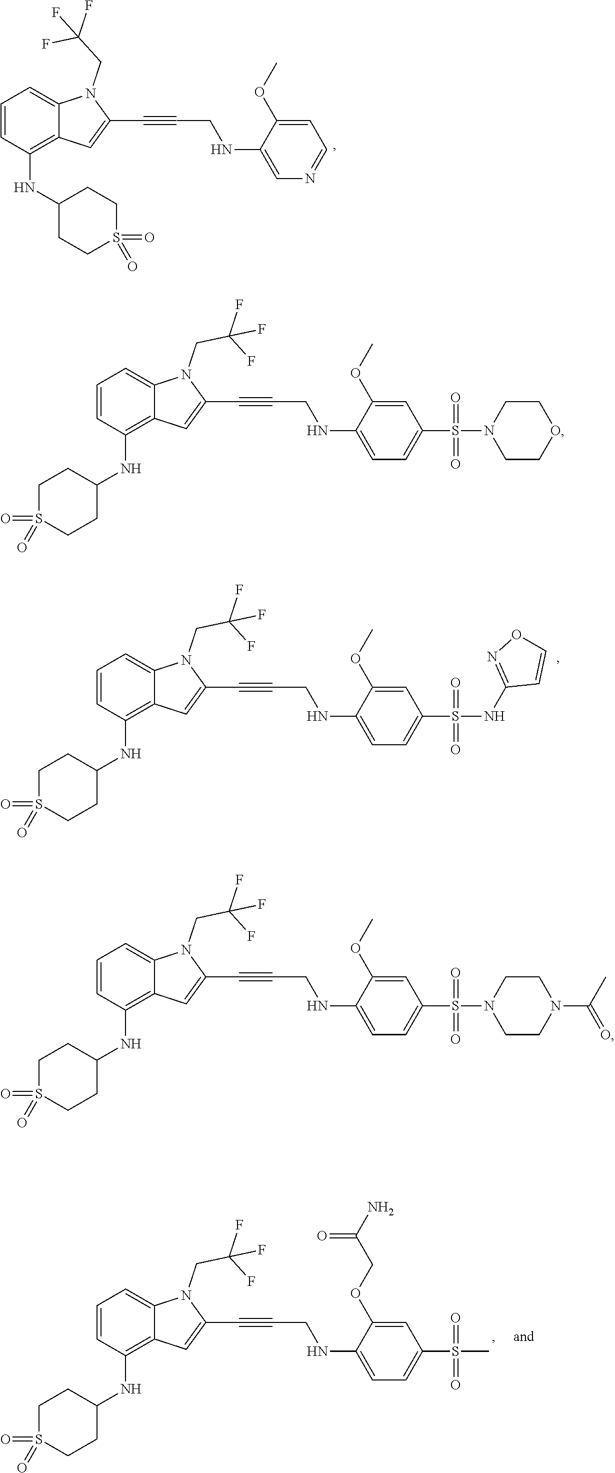 [SMILES: COc1ccncc1NCC#Cc1cc2c(NC3CCS(=O)(=O)CC3)cccc2n1CC(F)(F)F ; COc1cc(S(=O)(=O)N2CCOCC2)ccc1NCC#Cc1cc2c(NC3CCS(=O)(=O)CC3)cccc2n1CC(F)(F)F ; COc1cc(S(=O)(=O)Nc2ccon2)ccc1NCC#Cc1cc2c(NC3CCS(=O)(=O)CC3)cccc2n1CC(F)(F)F ; COc1cc(S(=O)(=O)N2CCN(C(C)=O)CC2)ccc1NCC#Cc1cc2c(NC3CCS(=O)(=O)CC3)cccc2n1CC(F)(F)F ; CS(=O)(=O)c1ccc(NCC#Cc2cc3c(NC4CCS(=O)(=O)CC4)cccc3n2CC(F)(F)F)c(OCC(N)=O)c1]

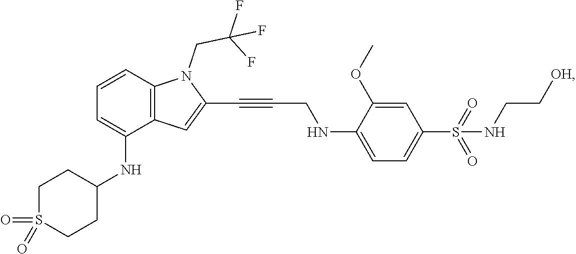 [SMILES: COc1cc(S(=O)(=O)NCCO)ccc1NCC#Cc1cc2c(NC3CCS(=O)(=O)CC3)cccc2n1CC(F)(F)F]

or a pharmaceutically-acceptable salt thereof.

Non-limiting examples of compounds of the current disclosure include the following:

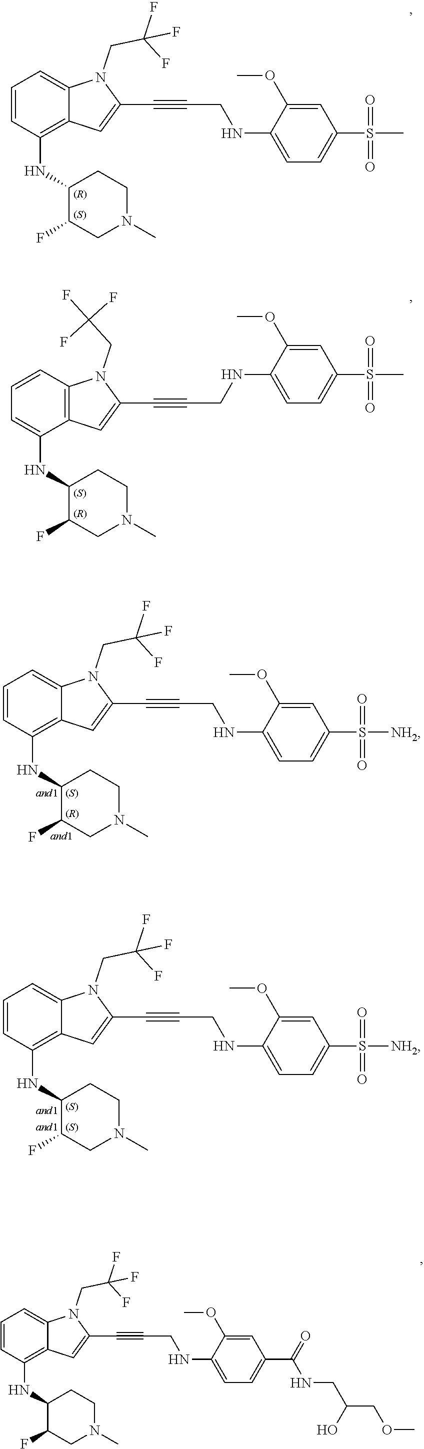

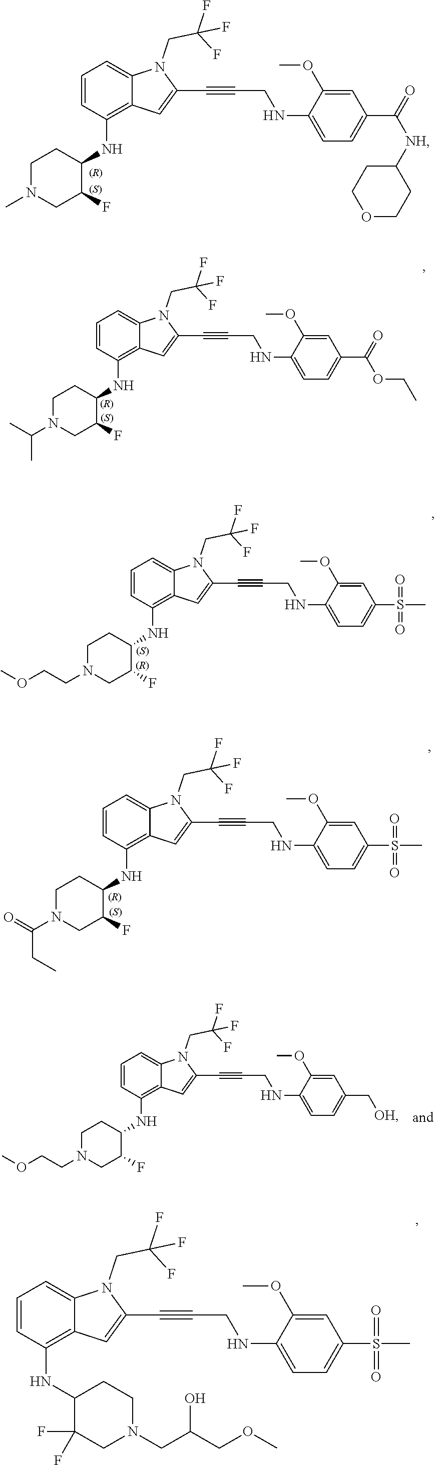

or a pharmaceutically-acceptable salt thereof.

Non-limiting examples of compounds of the current disclosure include the following:

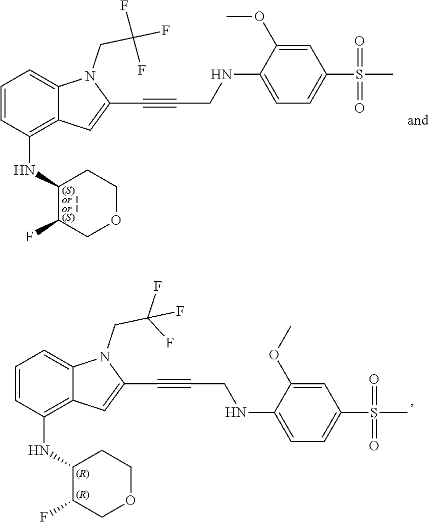

or a pharmaceutically-acceptable salt thereof.

Non-limiting examples of compounds of the current disclosure include the following:

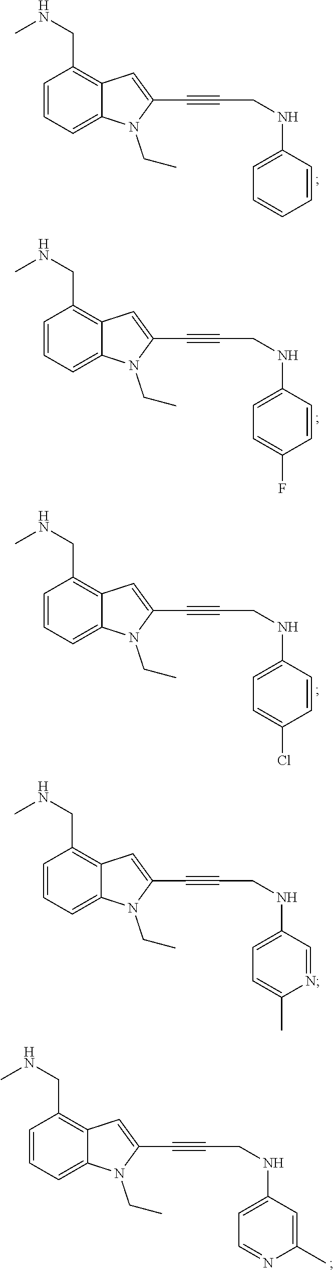

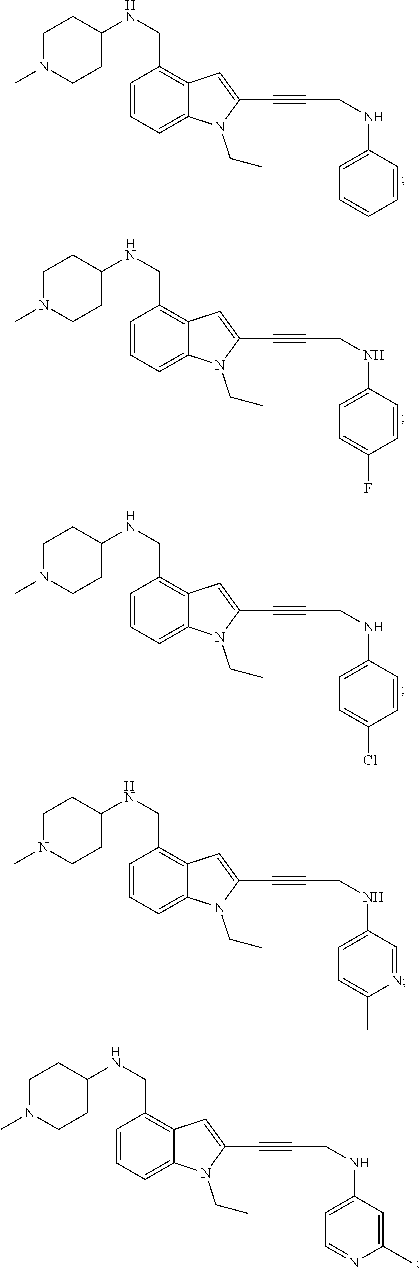

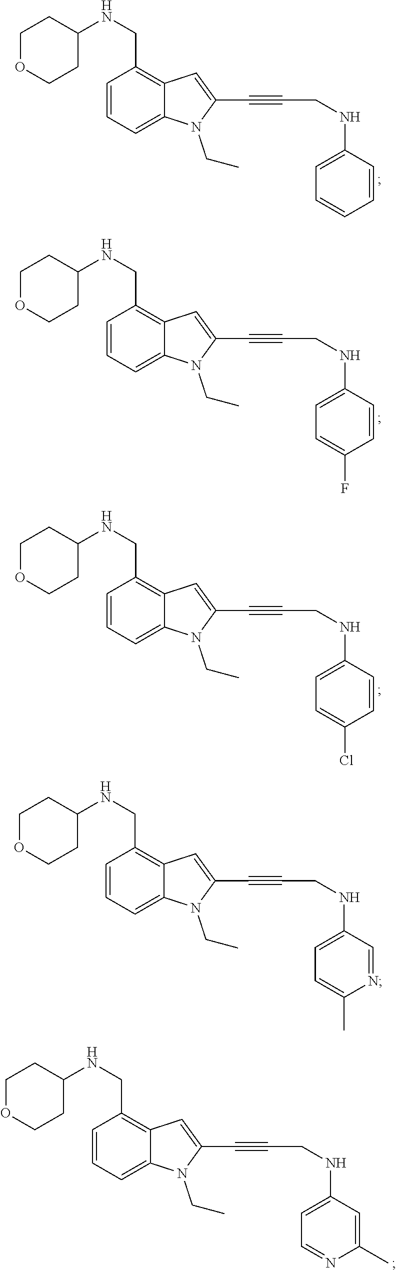 [SMILES: CCn1c(C#CCNc2ccccc2)cc2c(CNC3CCOCC3)cccc21 ; CCn1c(C#CCNc2ccc(F)cc2)cc2c(CNC3CCOCC3)cccc21 ; CCn1c(C#CCNc2ccc(Cl)cc2)cc2c(CNC3CCOCC3)cccc21 ; CCn1c(C#CCNc2ccc(C)nc2)cc2c(CNC3CCOCC3)cccc21 ; CCn1c(C#CCNc2ccnc(C)c2)cc2c(CNC3CCOCC3)cccc21]

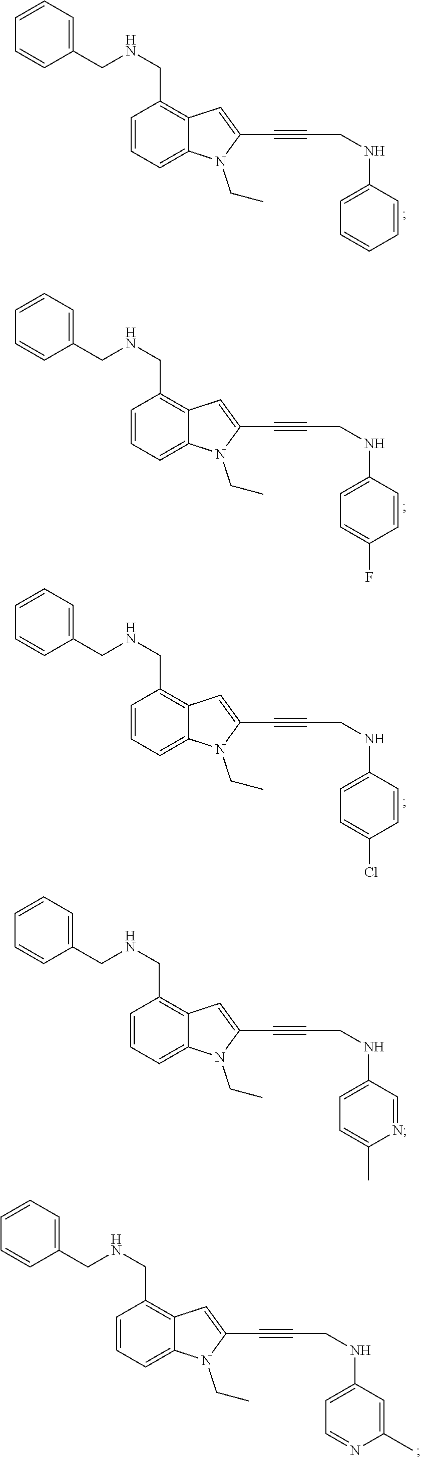 [SMILES: CCn1c(C#CCNc2ccccc2)cc2c(CNCc3ccccc3)cccc21 ; CCn1c(C#CCNc2ccc(F)cc2)cc2c(CNCc3ccccc3)cccc21 ; CCn1c(C#CCNc2ccc(Cl)cc2)cc2c(CNCc3ccccc3)cccc21 ; CCn1c(C#CCNc2ccc(C)nc2)cc2c(CNCc3ccccc3)cccc21 ; CCn1c(C#CCNc2ccnc(C)c2)cc2c(CNCc3ccccc3)cccc21]

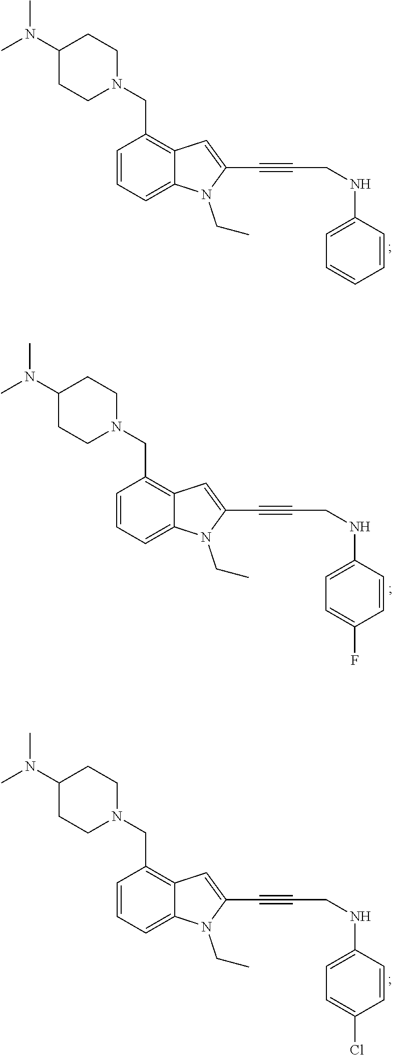 [SMILES: CCn1c(C#CCNc2ccccc2)cc2c(CN3CCC(N(C)C)CC3)cccc21 ; CCn1c(C#CCNc2ccc(F)cc2)cc2c(CN3CCC(N(C)C)CC3)cccc21 ; CCn1c(C#CCNc2ccc(Cl)cc2)cc2c(CN3CCC(N(C)C)CC3)cccc21]

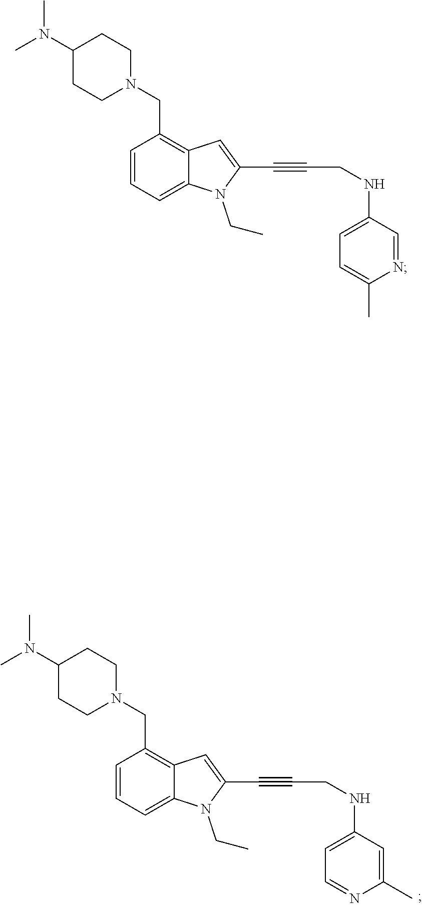

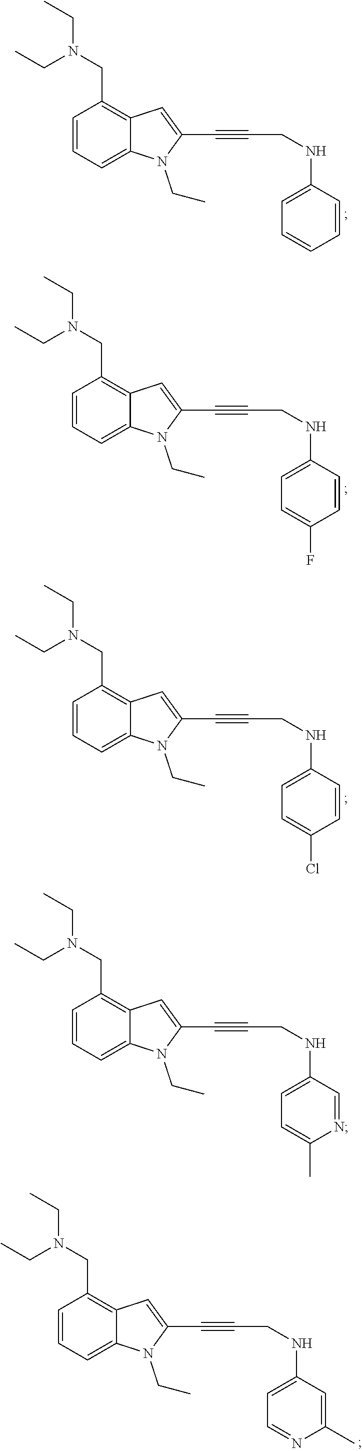 [SMILES: CCN(CC)Cc1cccc2c1cc(C#CCNc1ccccc1)n2CC ; CCN(CC)Cc1cccc2c1cc(C#CCNc1ccc(F)cc1)n2CC ; CCN(CC)Cc1cccc2c1cc(C#CCNc1ccc(Cl)cc1)n2CC ; CCN(CC)Cc1cccc2c1cc(C#CCNc1ccc(C)nc1)n2CC ; CCN(CC)Cc1cccc2c1cc(C#CCNc1ccnc(C)c1)n2CC]

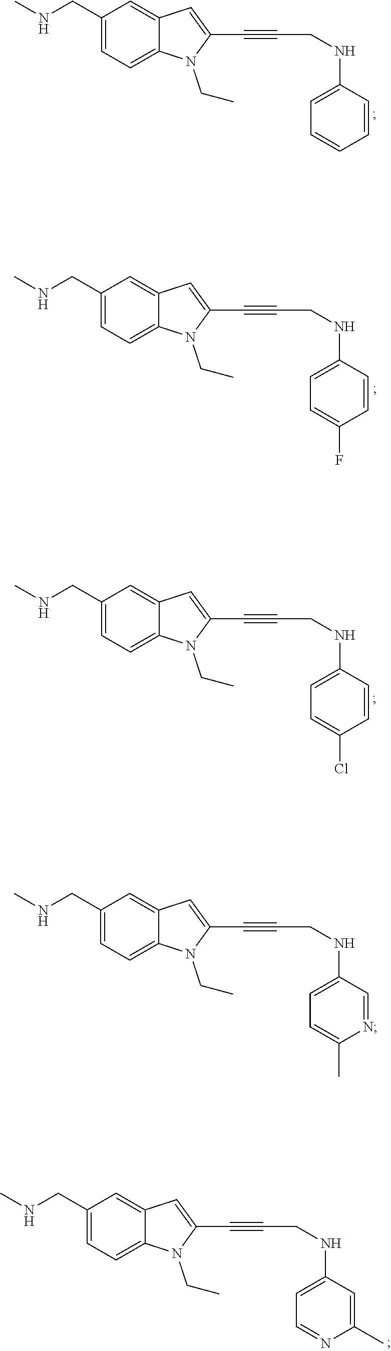

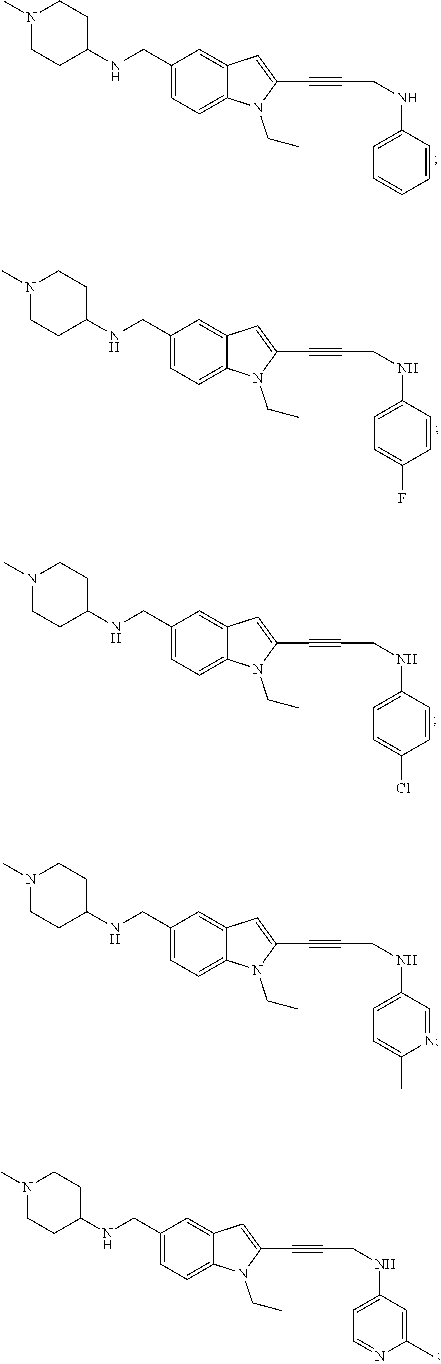

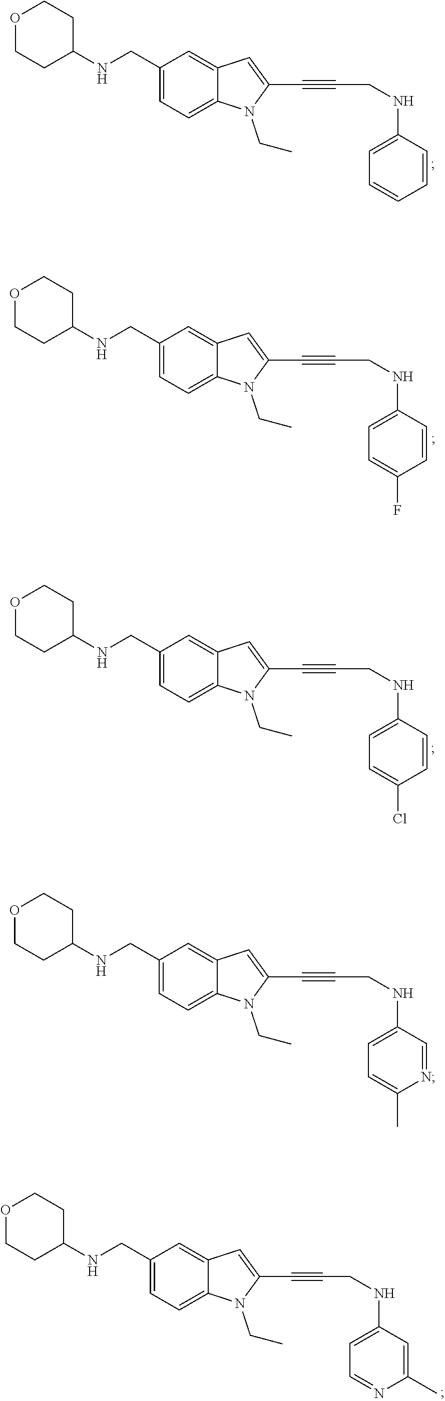 [SMILES: CCn1c(C#CCNc2ccccc2)cc2cc(CNC3CCOCC3)ccc21 ; CCn1c(C#CCNc2ccc(F)cc2)cc2cc(CNC3CCOCC3)ccc21 ; CCn1c(C#CCNc2ccc(Cl)cc2)cc2cc(CNC3CCOCC3)ccc21 ; CCn1c(C#CCNc2ccc(C)nc2)cc2cc(CNC3CCOCC3)ccc21 ; CCn1c(C#CCNc2ccnc(C)c2)cc2cc(CNC3CCOCC3)ccc21]

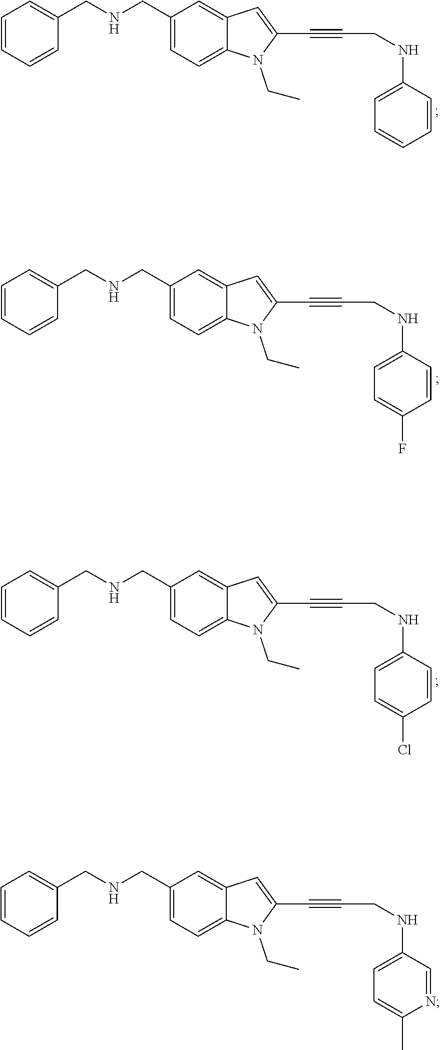 [SMILES: CCn1c(C#CCNc2ccccc2)cc2cc(CNCc3ccccc3)ccc21 ; CCn1c(C#CCNc2ccc(F)cc2)cc2cc(CNCc3ccccc3)ccc21 ; CCn1c(C#CCNc2ccc(Cl)cc2)cc2cc(CNCc3ccccc3)ccc21 ; CCn1c(C#CCNc2ccc(C)nc2)cc2cc(CNCc3ccccc3)ccc21]

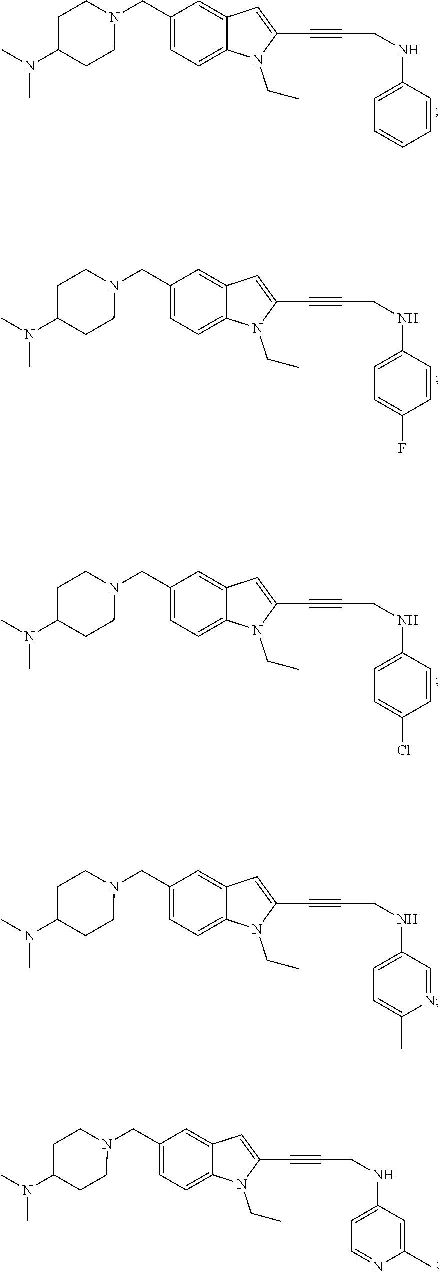

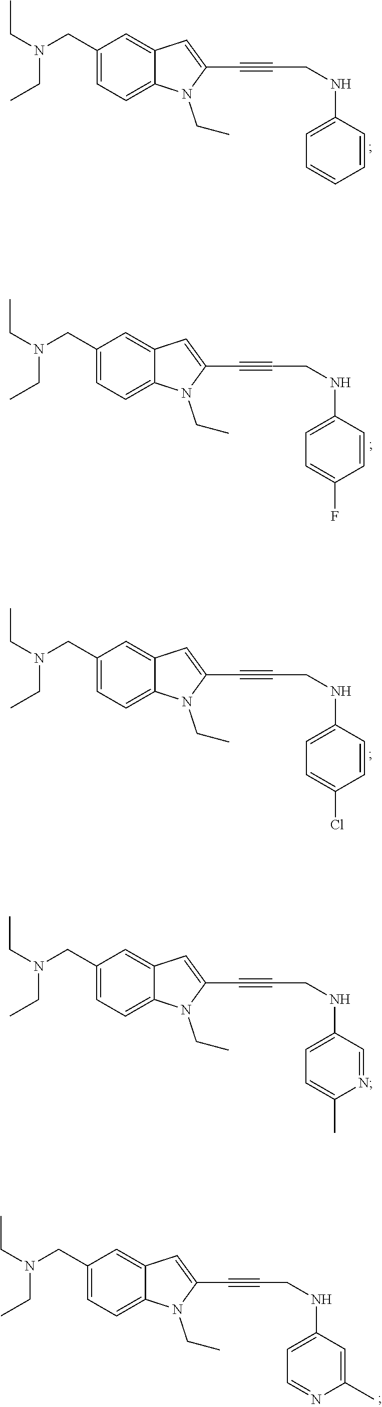 [SMILES: CCN(CC)Cc1ccc2c(c1)cc(C#CCNc1ccccc1)n2CC ; CCN(CC)Cc1ccc2c(c1)cc(C#CCNc1ccc(F)cc1)n2CC ; CCN(CC)Cc1ccc2c(c1)cc(C#CCNc1ccc(Cl)cc1)n2CC ; CCN(CC)Cc1ccc2c(c1)cc(C#CCNc1ccc(C)nc1)n2CC ; CCN(CC)Cc1ccc2c(c1)cc(C#CCNc1ccnc(C)c1)n2CC]

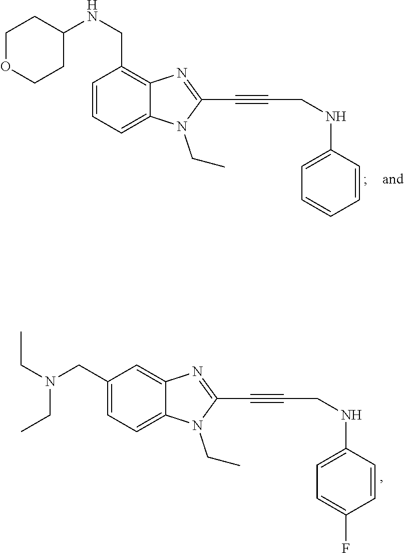 [SMILES: CCn1c(C#CCNc2ccccc2)nc2c(CNC3CCOCC3)cccc21 ; CCN(CC)Cc1ccc2c(c1)nc(C#CCNc1ccc(F)cc1)n2CC]

or a pharmaceutically-acceptable salt of any of the foregoing.

Non-limiting examples of compounds of the current disclosure include the following.

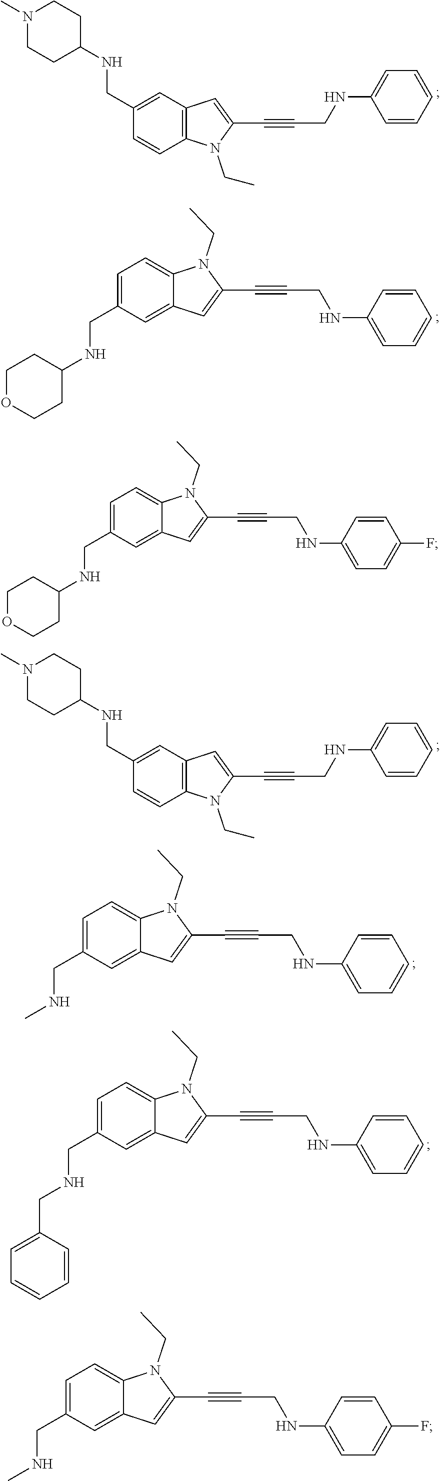

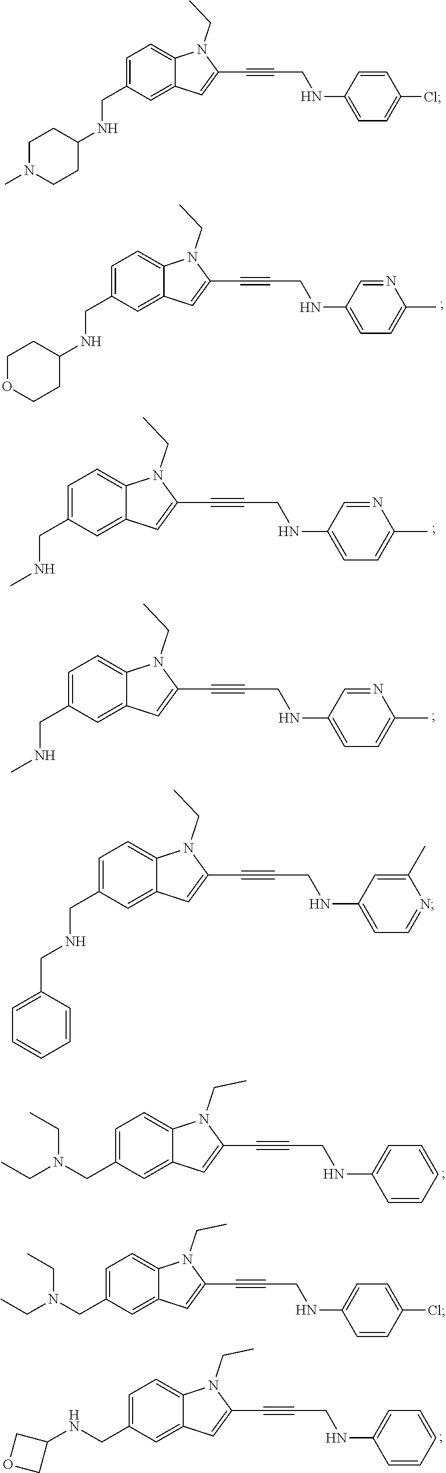

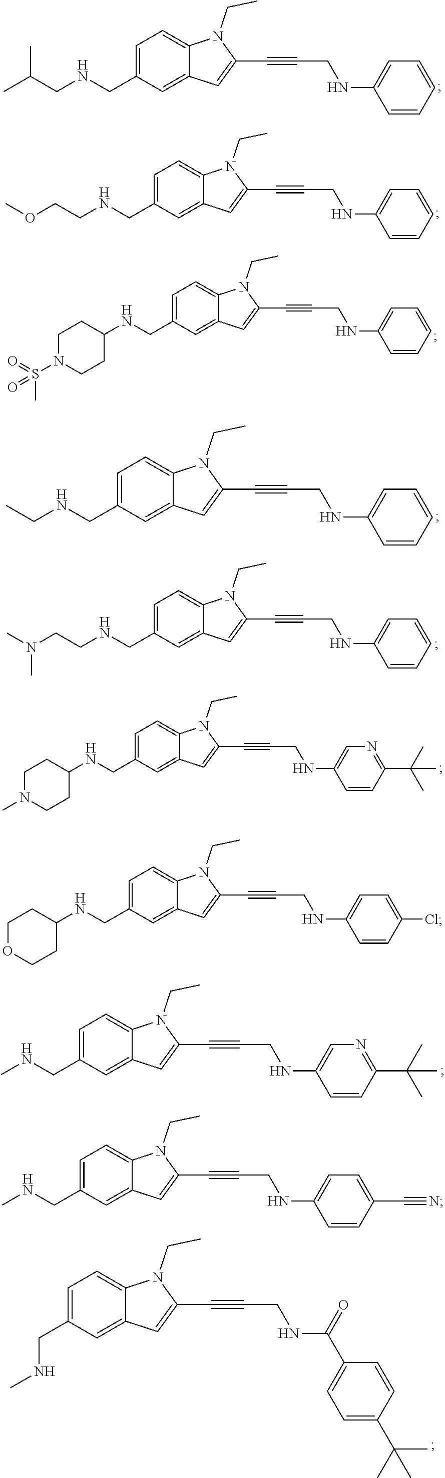

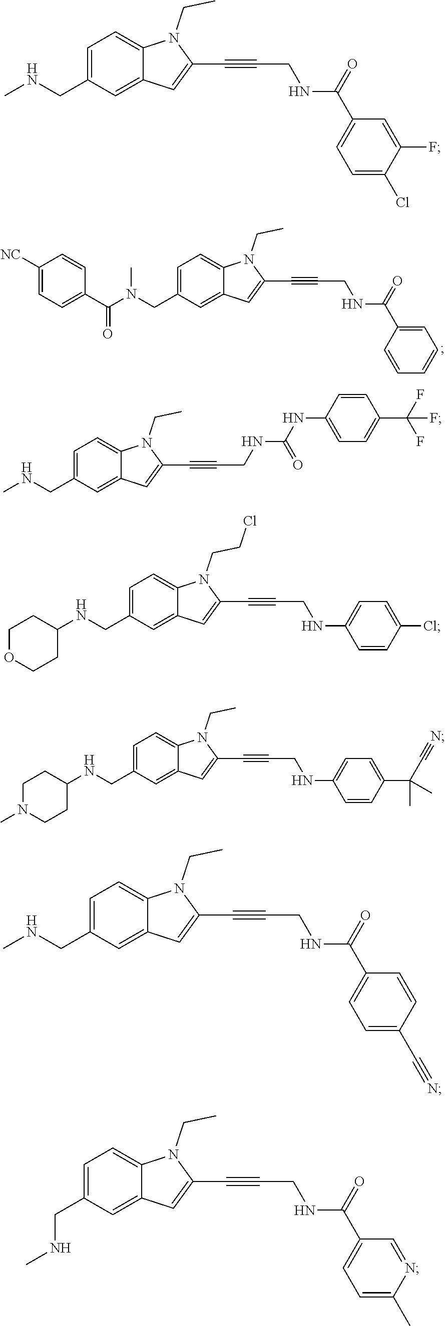

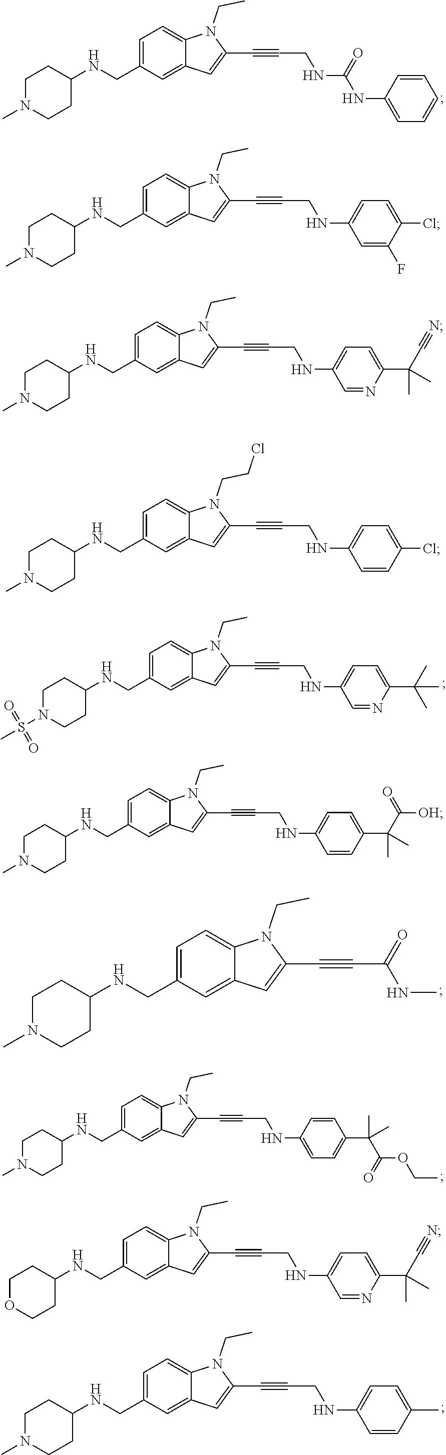

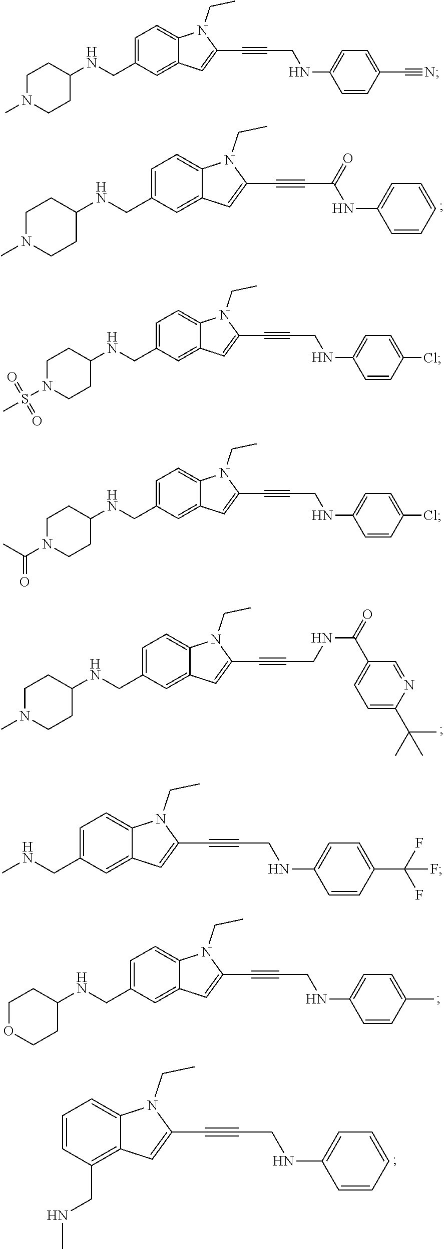

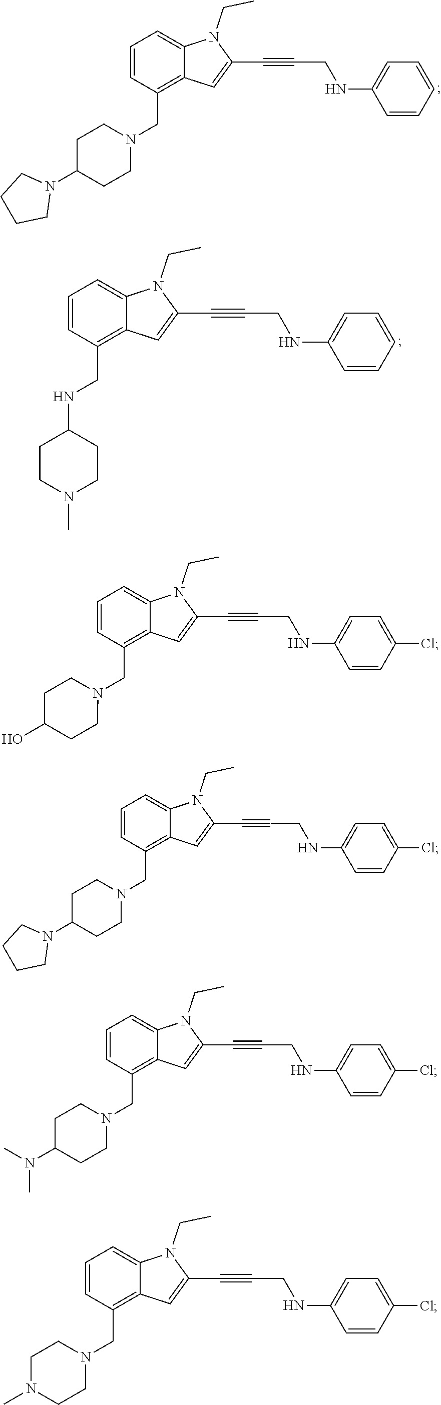 [SMILES: CCn1c(C#CCNc2ccccc2)cc2c(CN3CCC(N4CCCC4)CC3)cccc21 ; CCn1c(C#CCNc2ccccc2)cc2c(CNC3CCN(C)CC3)cccc21 ; CCn1c(C#CCNc2ccc(Cl)cc2)cc2c(CN3CCC(O)CC3)cccc21 ; CCn1c(C#CCNc2ccc(Cl)cc2)cc2c(CN3CCC(N4CCCC4)CC3)cccc21 ; CCn1c(C#CCNc2ccc(Cl)cc2)cc2c(CN3CCC(N(C)C)CC3)cccc21 ; CCn1c(C#CCNc2ccc(Cl)cc2)cc2c(CN3CCN(C)CC3)cccc21]

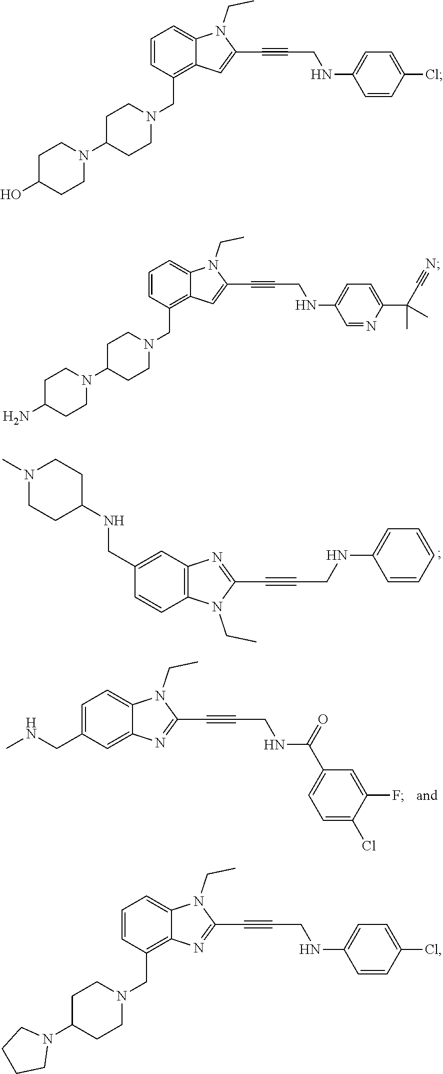

or a pharmaceutically-acceptable salt of any of the foregoing.

Non-limiting examples of compounds of the current disclosure include the following:

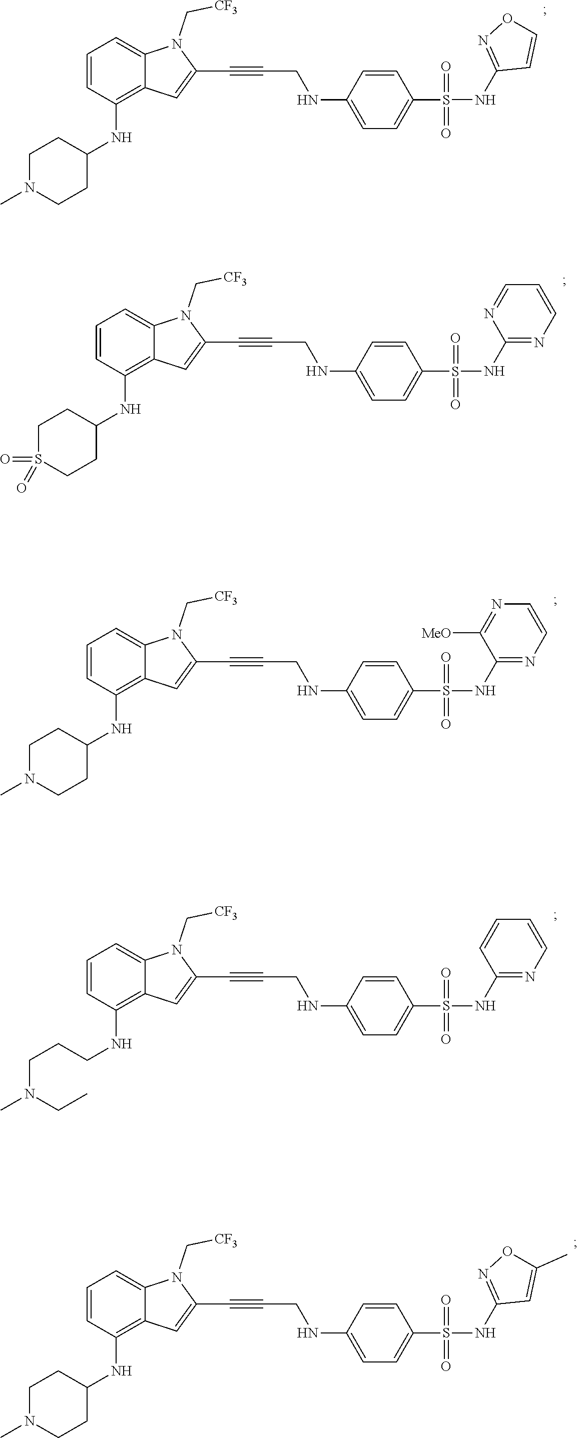

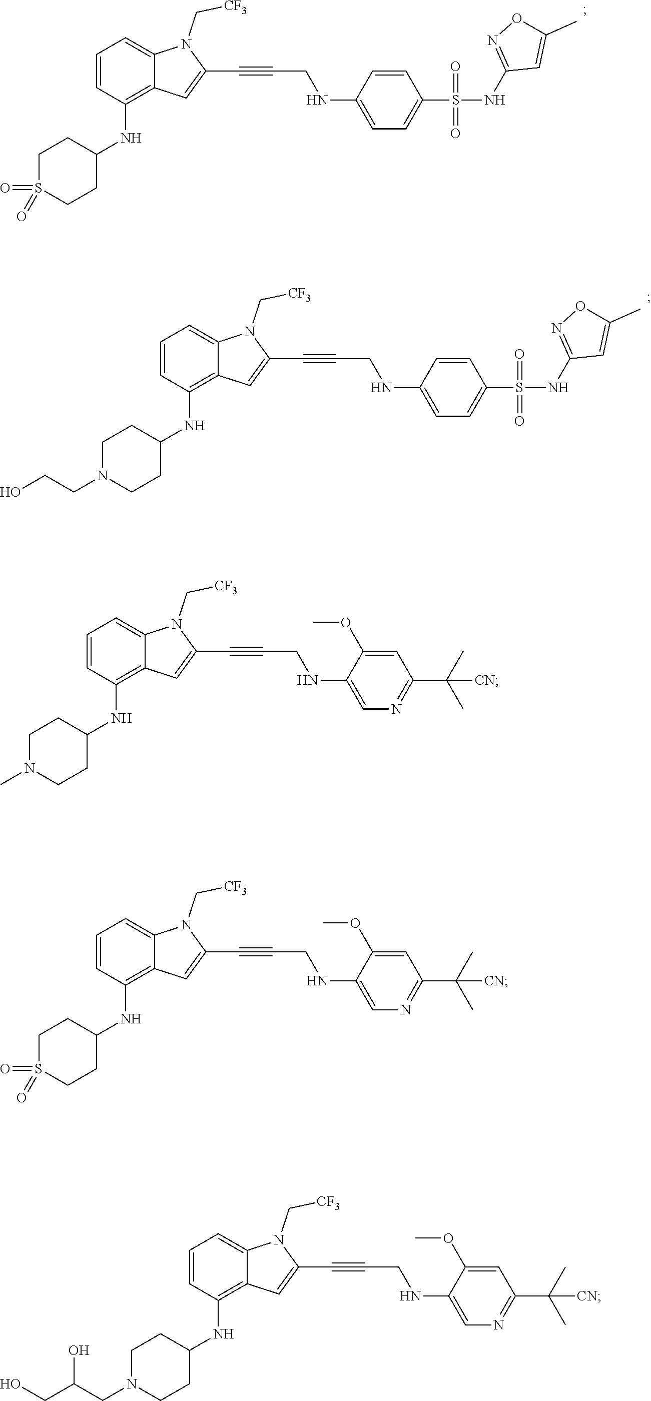

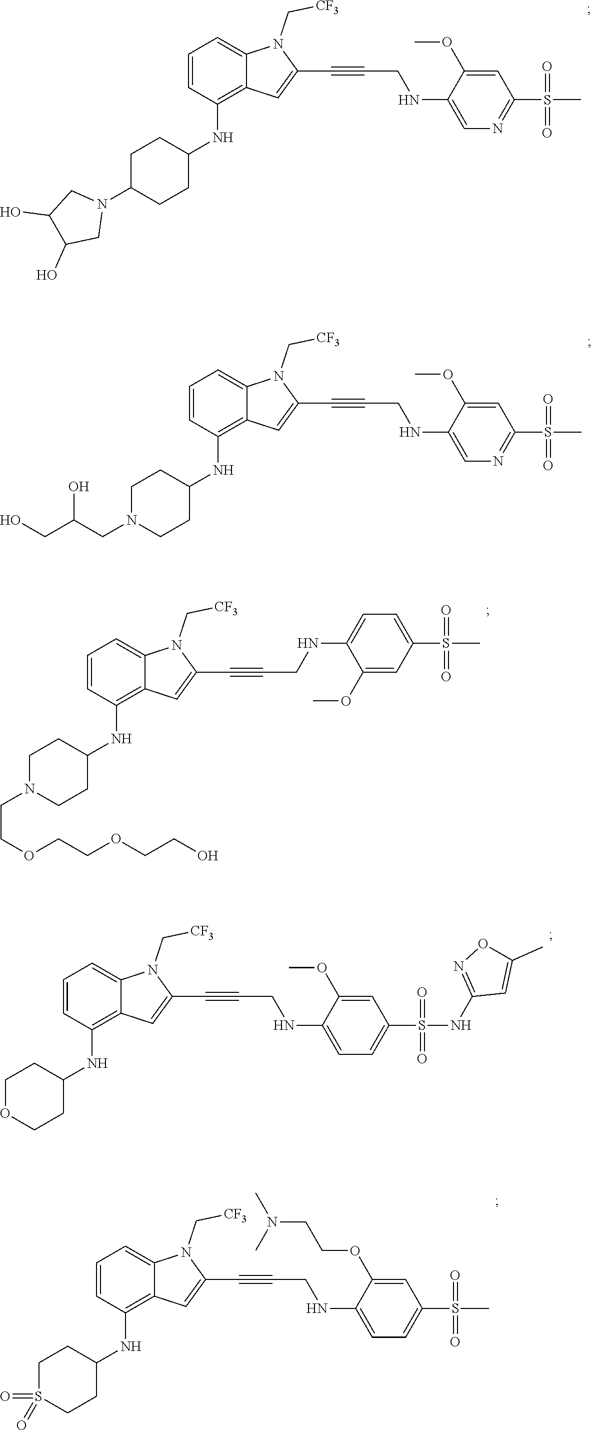 [SMILES: COc1cc(S(C)(=O)=O)ncc1NCC#Cc1cc2c(NC3CCC(N4CC(O)C(O)C4)CC3)cccc2n1CC(F)(F)F ; COc1cc(S(C)(=O)=O)ncc1NCC#Cc1cc2c(NC3CCN(CC(O)CO)CC3)cccc2n1CC(F)(F)F ; COc1cc(S(C)(=O)=O)ccc1NCC#Cc1cc2c(NC3CCN(CCOCCOCCO)CC3)cccc2n1CC(F)(F)F ; COc1cc(S(=O)(=O)Nc2cc(C)on2)ccc1NCC#Cc1cc2c(NC3CCOCC3)cccc2n1CC(F)(F)F ; CN(C)CCOc1cc(S(C)(=O)=O)ccc1NCC#Cc1cc2c(NC3CCS(=O)(=O)CC3)cccc2n1CC(F)(F)F]

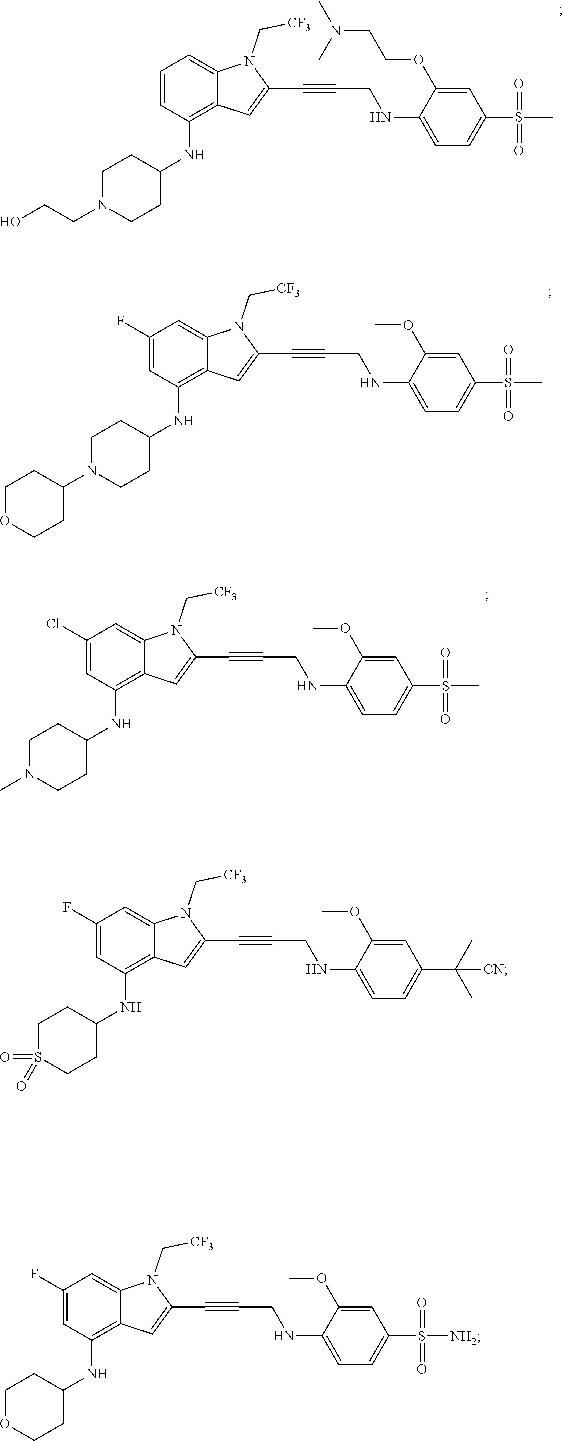

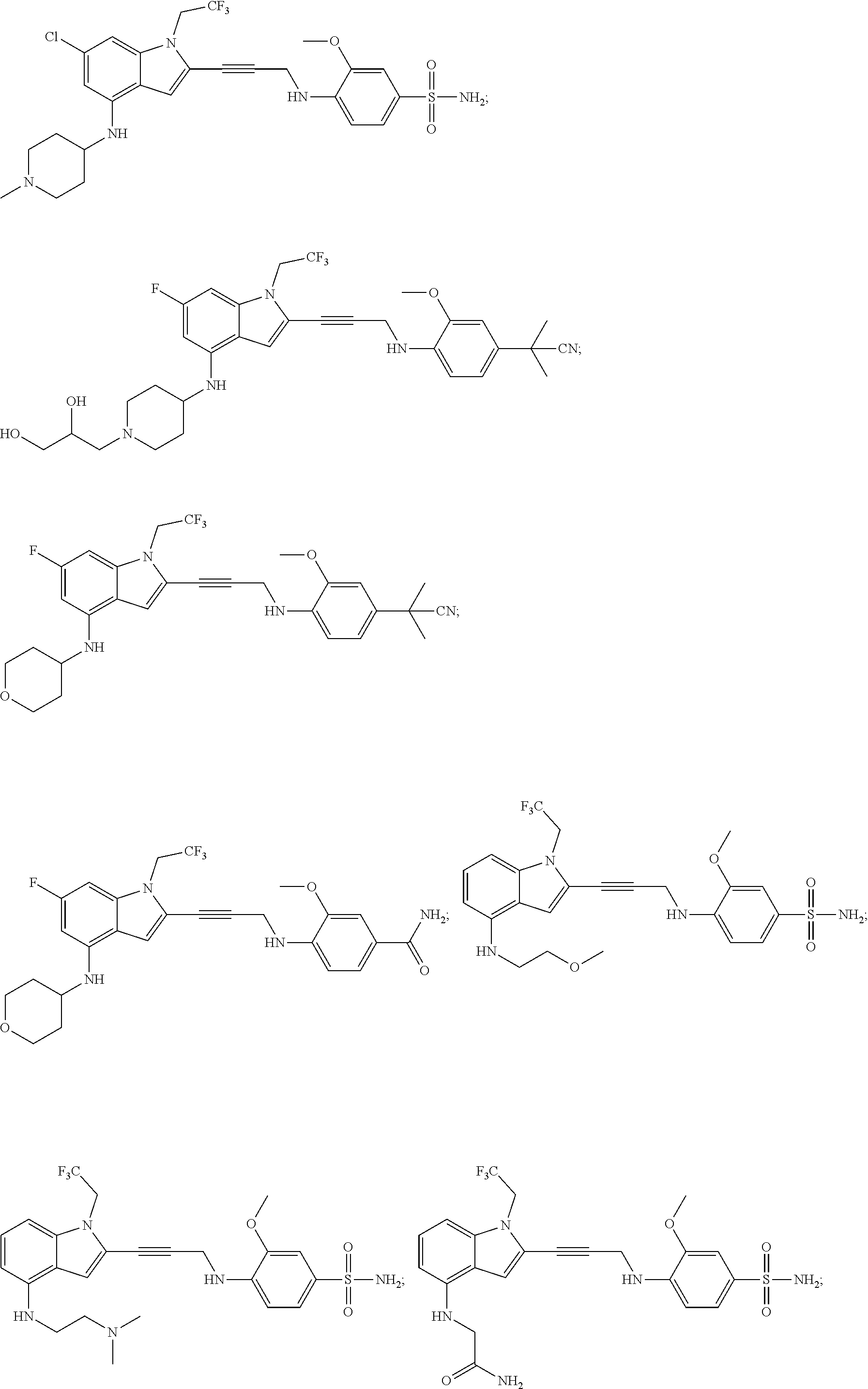

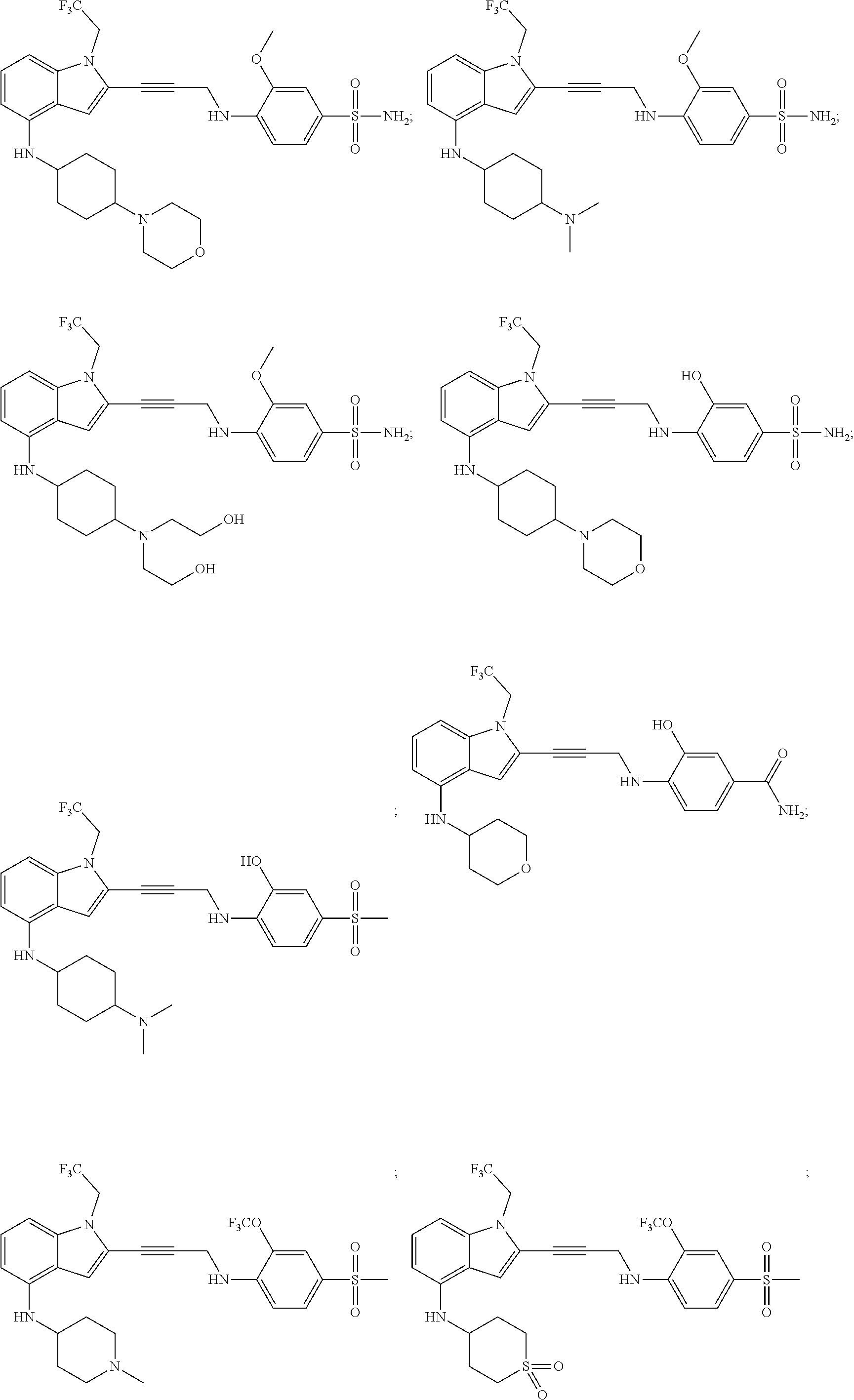

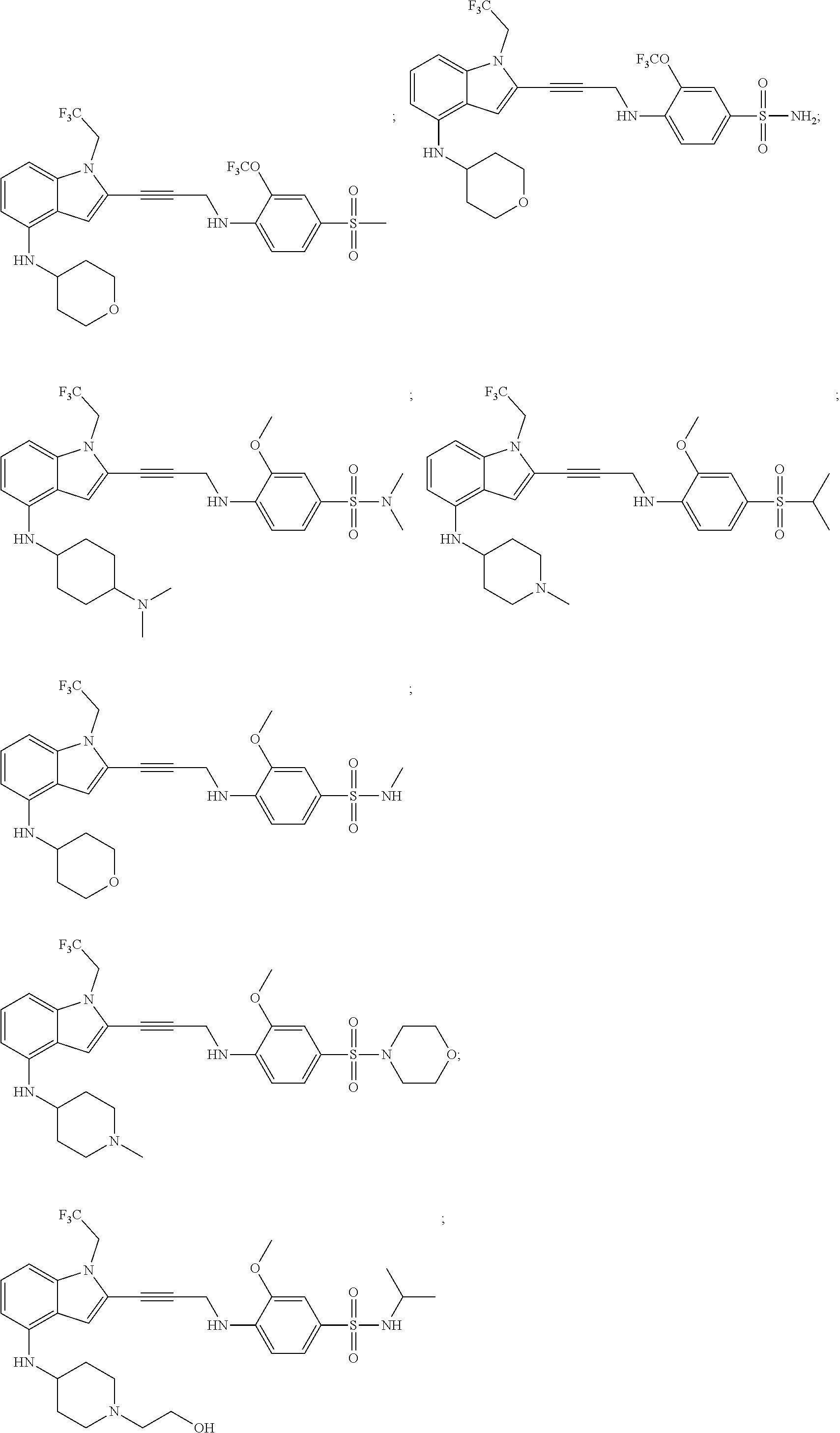

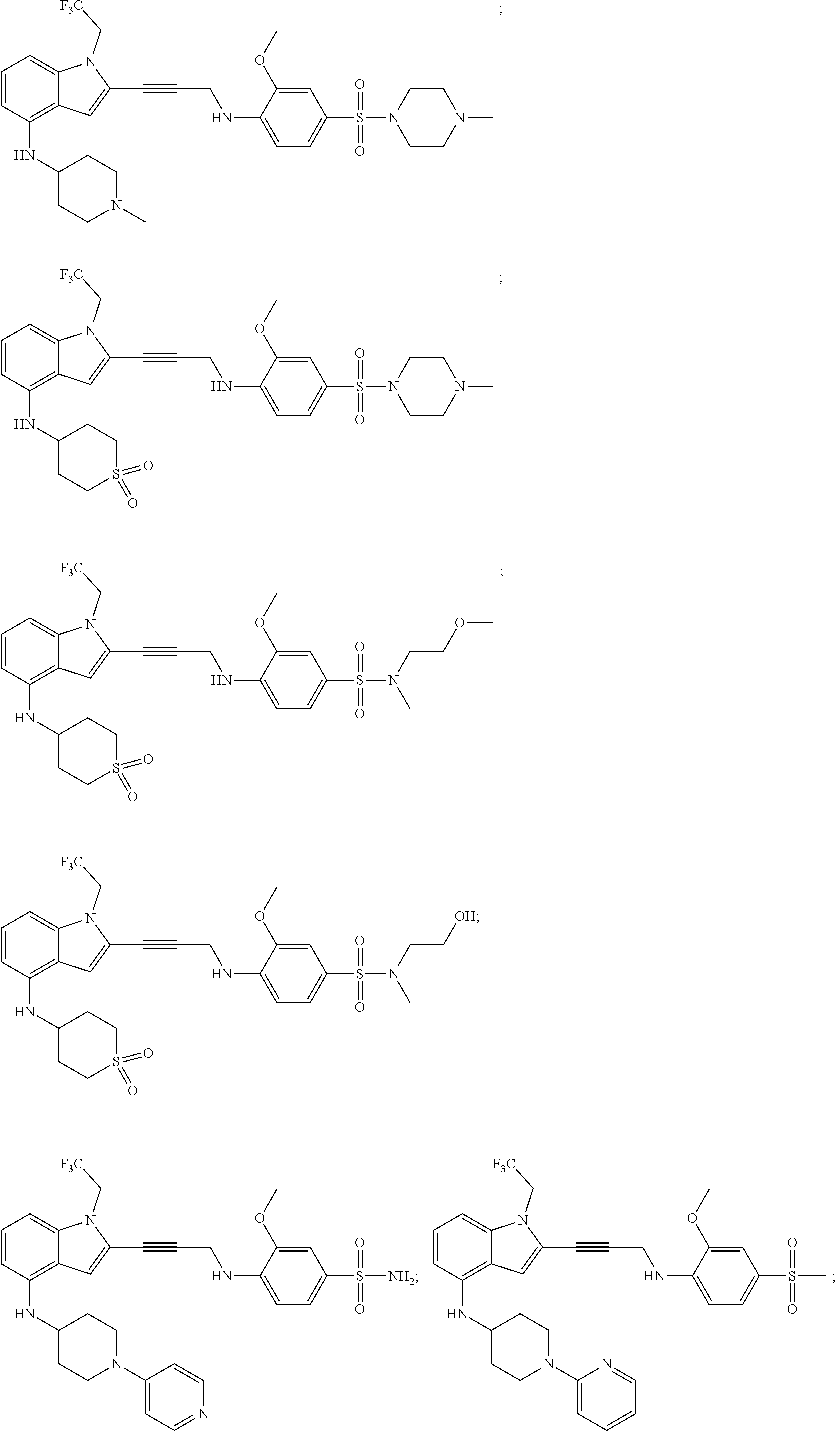

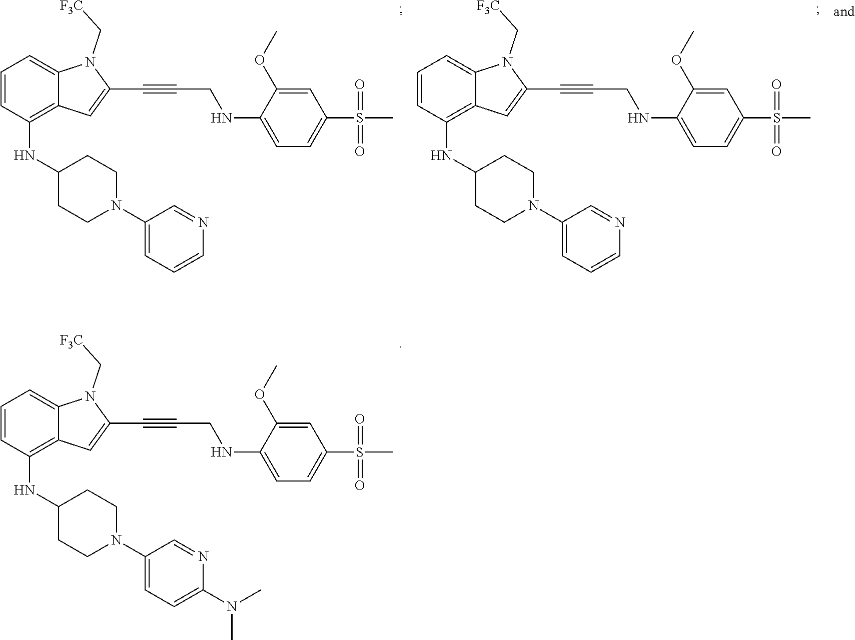

or a pharmaceutically-acceptable salt of any of the forgoing.

Non-limiting examples of compounds of the current disclosure include the following:

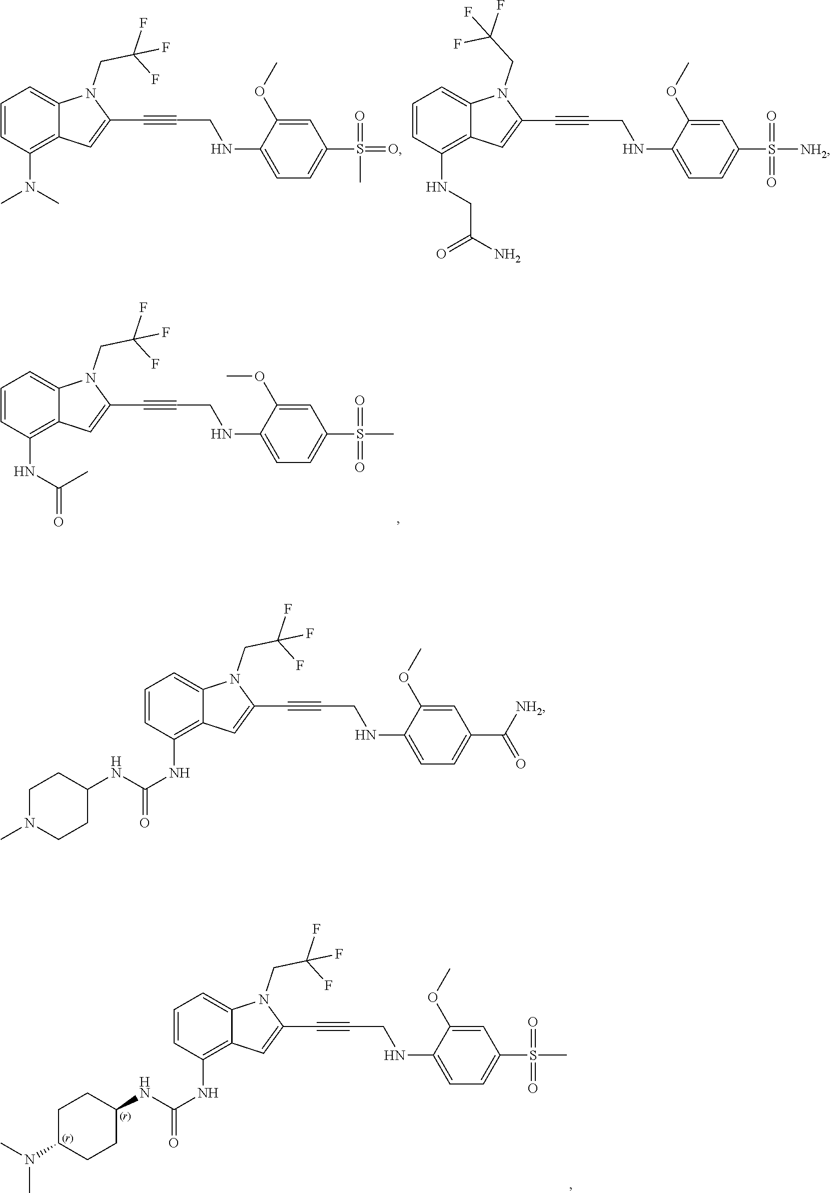

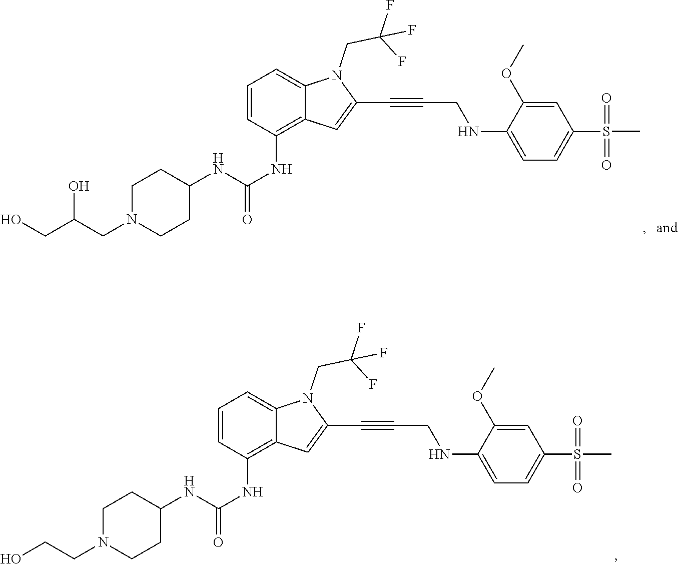 [SMILES: COc1cc(S(C)(=O)=O)ccc1NCC#Cc1cc2c(NC(=O)NC3CCN(CC(O)CO)CC3)cccc2n1CC(F)(F)F ; COc1cc(S(C)(=O)=O)ccc1NCC#Cc1cc2c(NC(=O)NC3CCN(CCO)CC3)cccc2n1CC(F)(F)F]

or a pharmaceutically-acceptable salt thereof.

Non-limiting examples of compounds of the current disclosure include the following:

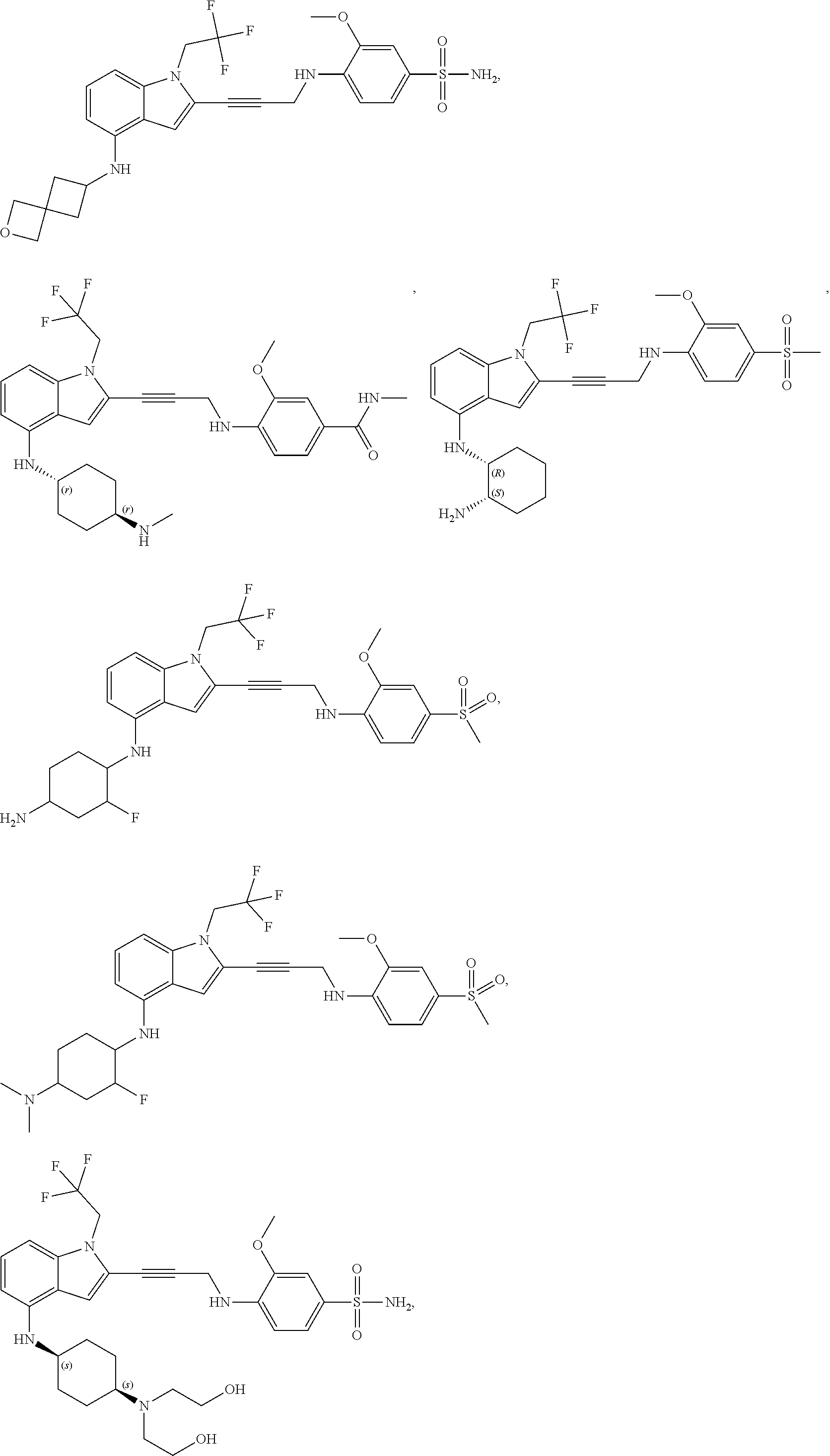

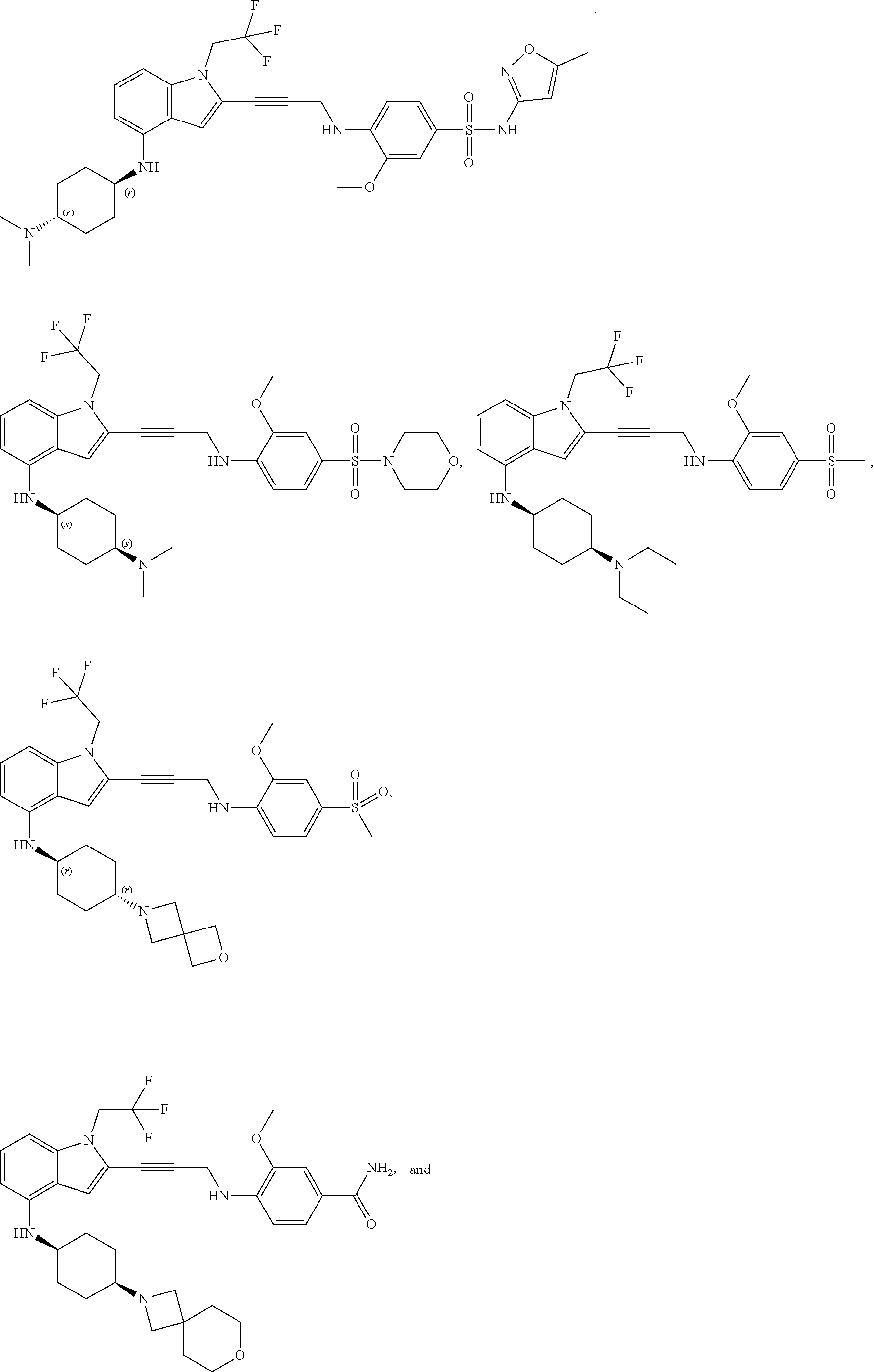

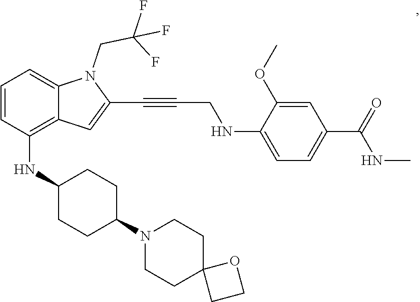 [SMILES: CNC(=O)c1ccc(NCC#Cc2cc3c(N[C@H]4CC[C@@H](N5CCC6(CCO6)CC5)CC4)cccc3n2CC(F)(F)F)c(OC)c1]

or a pharmaceutically-acceptable salt thereof.

Non-limiting examples of compounds of the current disclosure include the following:

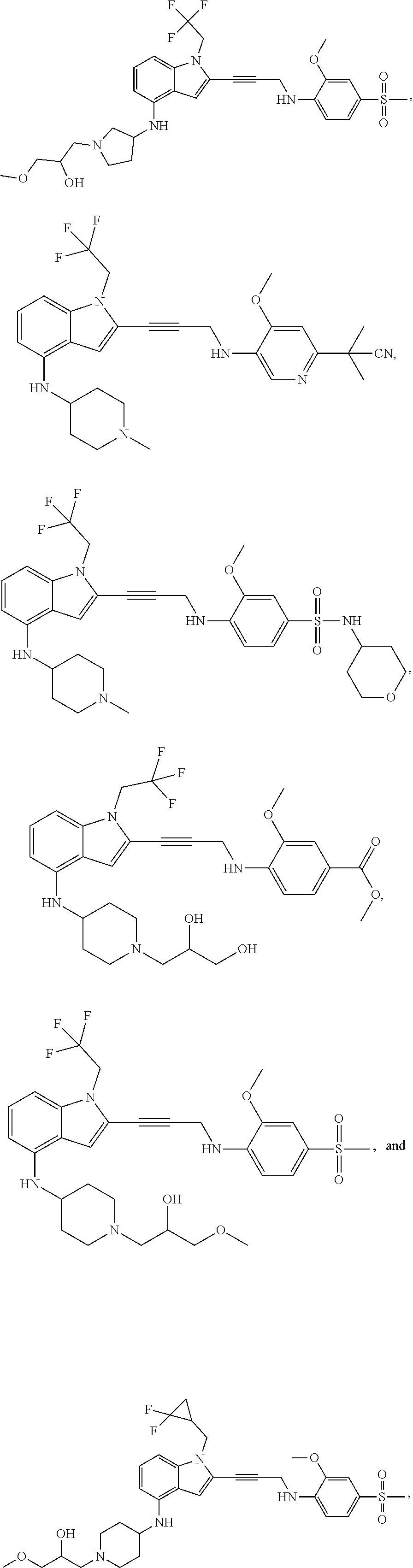

or a pharmaceutically-acceptable salt thereof.

Non-limiting examples of compounds of the current disclosure include the following:

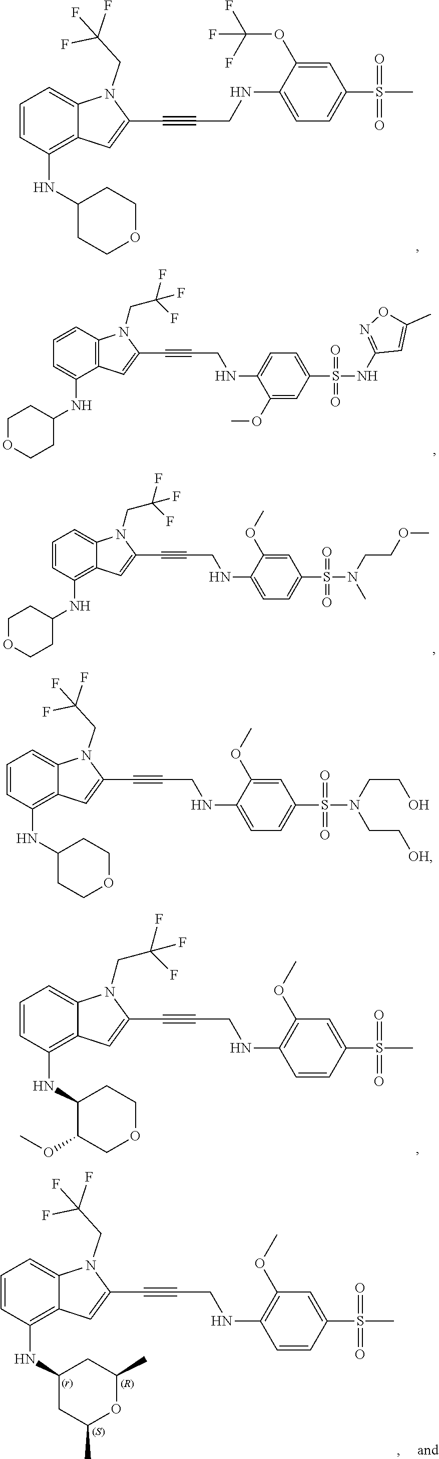

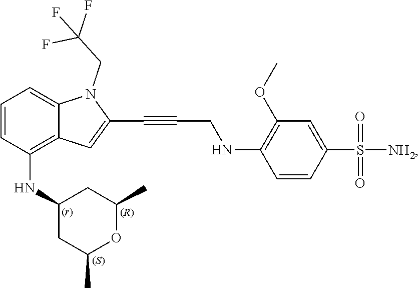

or a pharmaceutically-acceptable salt thereof.

Non-limiting examples of compounds of the current disclosure include the following.

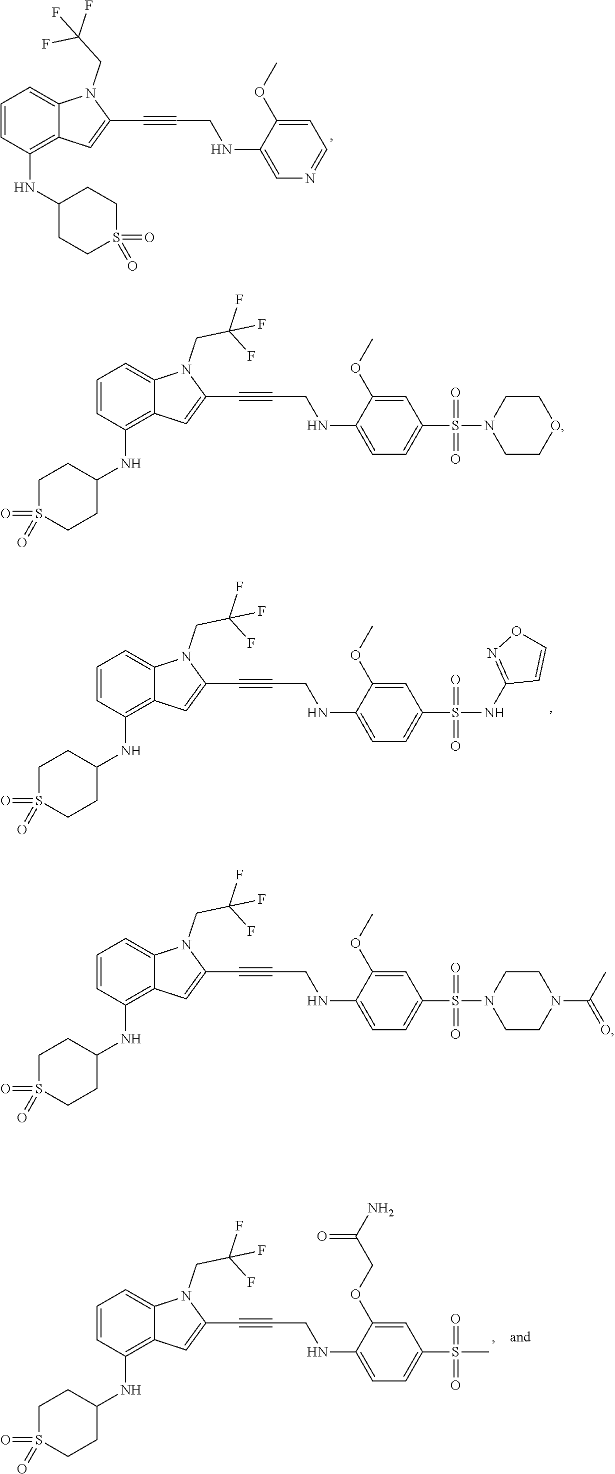 [SMILES: COc1ccncc1NCC#Cc1cc2c(NC3CCS(=O)(=O)CC3)cccc2n1CC(F)(F)F ; COc1cc(S(=O)(=O)N2CCOCC2)ccc1NCC#Cc1cc2c(NC3CCS(=O)(=O)CC3)cccc2n1CC(F)(F)F ; COc1cc(S(=O)(=O)Nc2ccon2)ccc1NCC#Cc1cc2c(NC3CCS(=O)(=O)CC3)cccc2n1CC(F)(F)F ; COc1cc(S(=O)(=O)N2CCN(C(C)=O)CC2)ccc1NCC#Cc1cc2c(NC3CCS(=O)(=O)CC3)cccc2n1CC(F)(F)F ; CS(=O)(=O)c1ccc(NCC#Cc2cc3c(NC4CCS(=O)(=O)CC4)cccc3n2CC(F)(F)F)c(OCC(N)=O)c1]

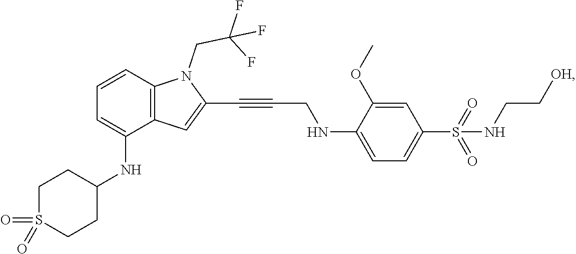 [SMILES: COc1cc(S(=O)(=O)NCCO)ccc1NCC#Cc1cc2c(NC3CCS(=O)(=O)CC3)cccc2n1CC(F)(F)F]

or a pharmaceutically-acceptable salt thereof.

Non-limiting examples of compounds of the current disclosure include the following:

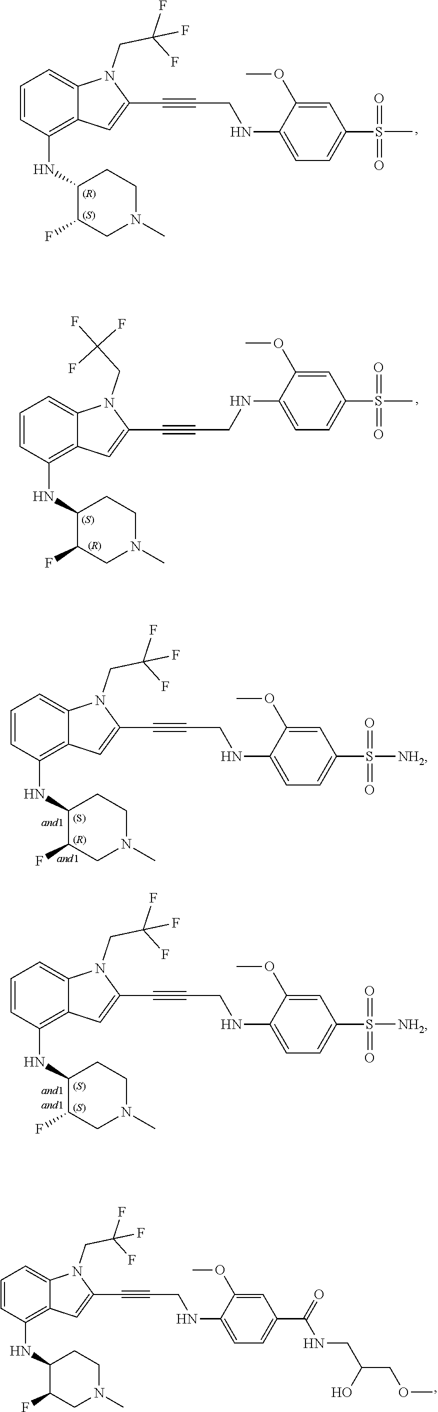

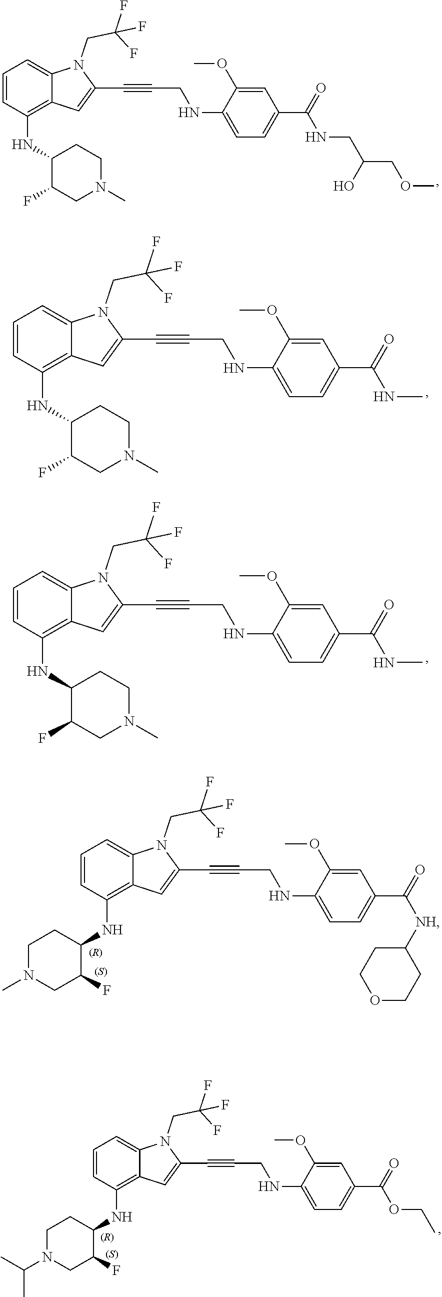

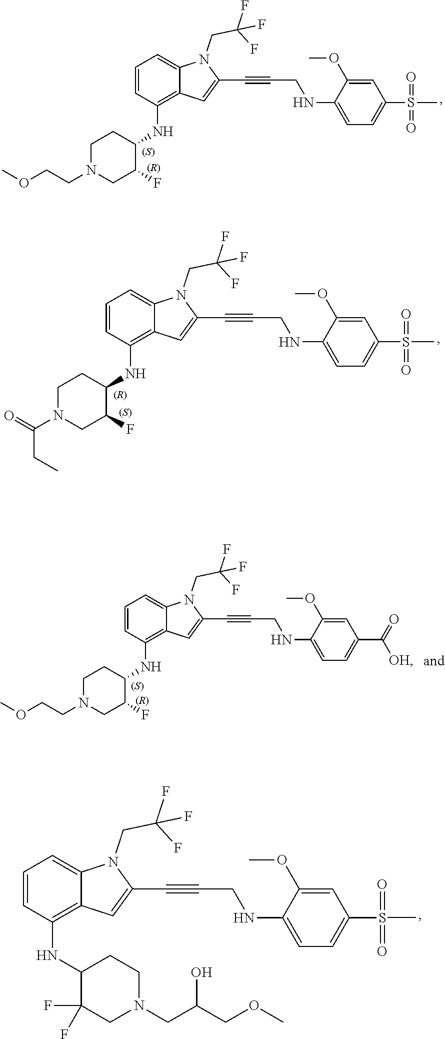

or a pharmaceutically-acceptable salt thereof.

Non-limiting examples of compounds of the current disclosure include the following:

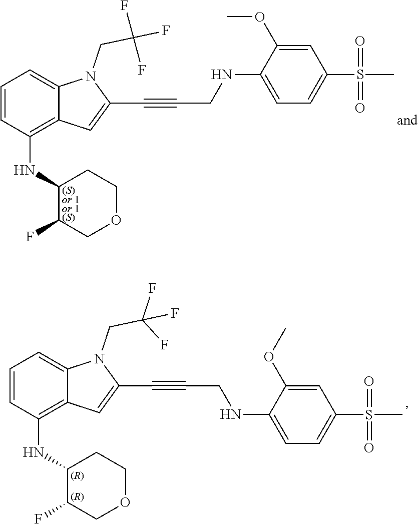

or a pharmaceutically-acceptable salt thereof.

In some embodiments, the disclosure provides a compound comprising: an indole group, wherein the indole group comprises: a) a haloalkyl group at a 1-position of the indole group; b) a first substituent at a 2-position of the indole group, wherein the first substituent is a cyclic group; and c) a second substituent, wherein the second substituent is substituted with at least halo-; or a pharmaceutically-acceptable salt thereof.

In some embodiments, the cyclic group is aryl, heteroaryl, or heterocyclyl, each of which is substituted or unsubstituted. In some embodiments, the cyclic group is unsubstituted aryl. In some embodiments, the cyclic group is substituted aryl. In some embodiments, the cyclic group is substituted phenyl. In some embodiments, the cyclic group is substituted or unsubstituted heteroaryl. In some embodiments, the heteroaryl is an aromatic 5-membered or 6-membered monocyclic ring. In some embodiments, the heteroaryl is thiazolyl, thiadiazolyl, pyrazolyl, thiophenyl, or oxadiazolyl. In some embodiments, the heteroaryl is pyridinyl or pyrimidinyl.

In some embodiments, the second substituent is at a 4-position of the indole group. In some embodiments, the second substituent is a second cyclic group that is substituted or unsubstituted. In some embodiments, the second cyclic group is heterocyclyl. In some embodiments, the heterocyclyl is piperidinyl. In some embodiments, the heterocyclyl is tetrahydropyranyl. In some embodiments, the heterocyclyl is substituted with fluoro-. In some embodiments, the heterocyclyl is substituted with chloro-. In some embodiments, the haloalkyl group is trifluoroethyl.

In some embodiments, the disclosure provides a compound, the compound comprising an indole group, wherein the indole group comprises: a) a substituted or unsubstituted non-cyclic group at a 3-positon of the indole group; and b) a substituted or unsubstituted cyclic group at a 2-position of the indole group, wherein the compound increases a stability of a biologically-active conformation of a p53 mutant relative to a stability of a biologically-active conformation of the p53 mutant in an absence of the compound, or a pharmaceutically-acceptable salt thereof.

In some embodiments, the non-cyclic group is hydrogen. In some embodiments, the non-cyclic group is halo-. In some embodiments, the cyclic group is aryl, heteroaryl, heterocyclyl, or cycloalkylene, each of which is substituted or unsubstituted. In some embodiments, the cyclic group is aryl or heteroaryl, each of which is substituted or unsubstituted. In some embodiments, the cyclic group is substituted aryl. In some embodiments, the cyclic group is substituted phenyl. In some embodiments, the cyclic group is phenyl substituted with alkyl, cycloalkyl, alkoxy, an amine group, a carboxyl group, a carboxylic acid group, a carbamide group, or an amide group, each of which is substituted or unsubstituted; cyano, halo-, or hydrogen.

In some embodiments, the cyclic group is substituted heteroaryl. In some embodiments, the cyclic group is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, the cyclic group is pyridinyl, pyrimidinyl, thiadiazolyl, thiazolyl, pyrazolyl, thiophenyl, or oxadiazolyl, In some embodiments, the cyclic group is 1,3,5-thiadiazol-2-yl. In some embodiments, the cyclic group is 1,3,4-oxadiazol-2-yl or 1,2,4-oxadiazol-2-yl. In some embodiments, the cyclic group is pyridinyl.

In some embodiments, the indole group further comprises a substituent at a 4-position of the indole group. In some embodiments, the substituent is an amino group that is substituted or unsubstituted. In some embodiments, the amino group is substituted with a second cyclic group. In some embodiments, the second cyclic group is a heterocyclyl group substituted with at least halo-. In some embodiments, the heterocyclyl group is substituted with at least fluoro-. In some embodiments, the heterocyclyl group is substituted with at least chloro-. In some embodiments, the heterocyclyl group is piperidinyl. In some embodiments, the heterocyclyl group is tetrahydropyranyl.

Non-limiting examples of compounds of the disclosure include compounds of any of the following formulae:

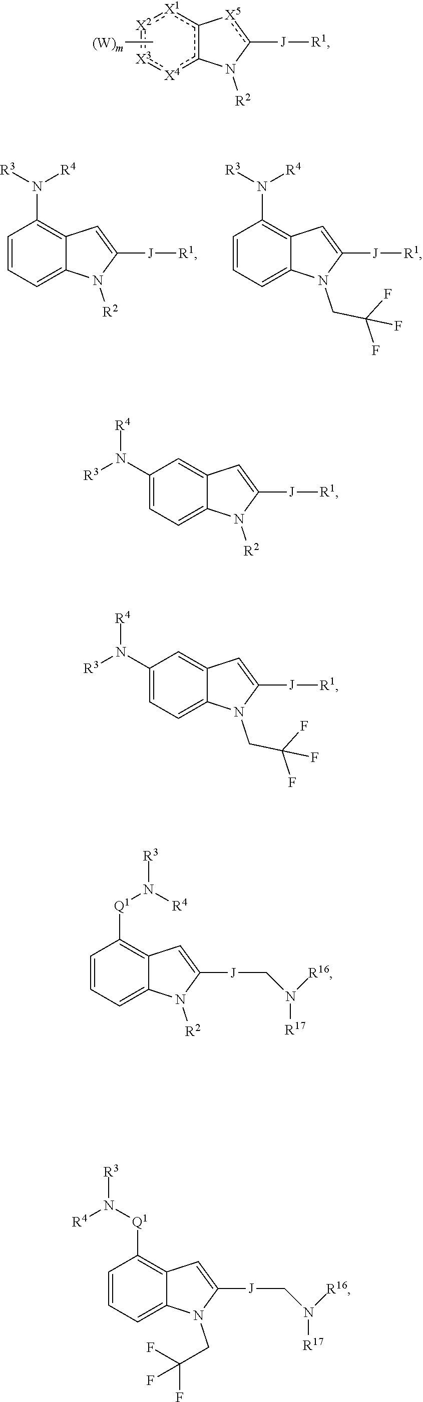

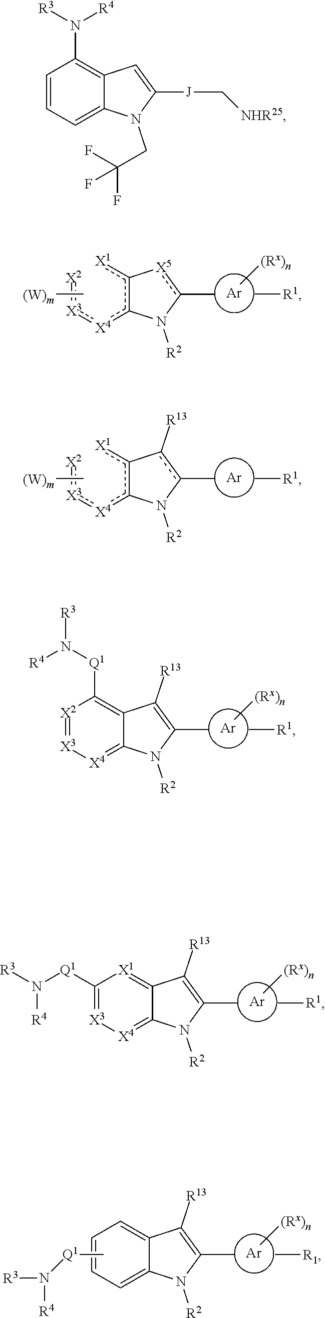

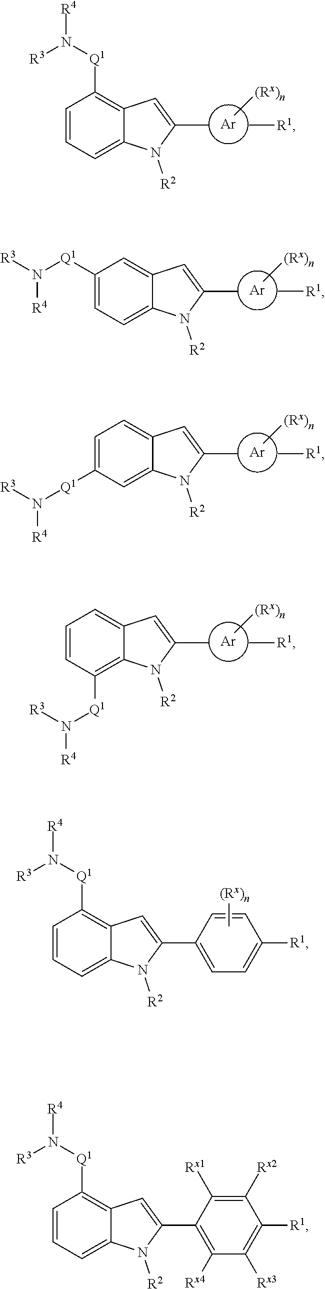

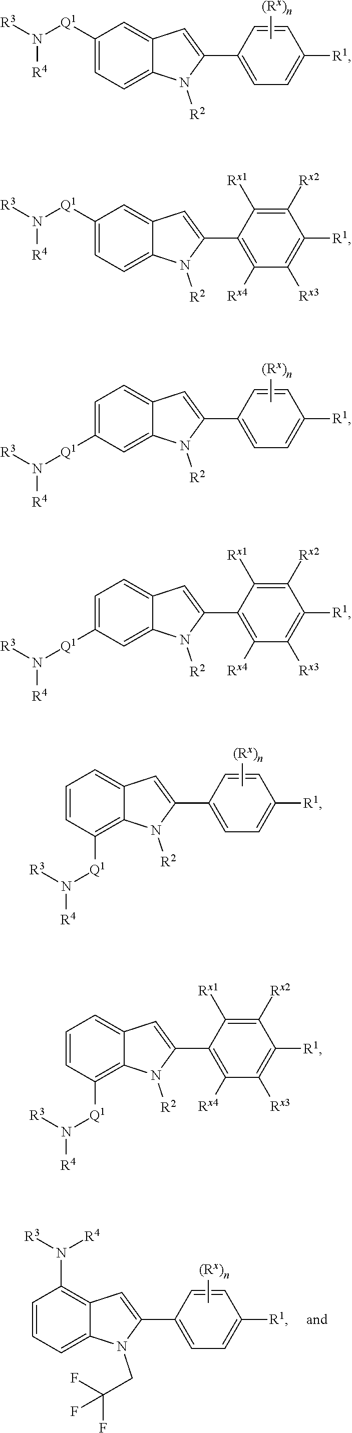

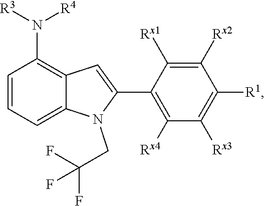

or a pharmaceutically-acceptable salt thereof.

In some embodiments, the disclosure provides a compound of the formula:

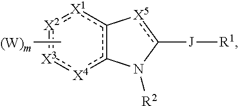

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each W is independently -Q¹-N(R³)R⁴, -Q¹-OR⁴, or -Q¹-R⁴;
   wherein at least one of X¹, X², X³, and X⁴ is a carbon atom connected to Q¹;
J is a substituted or unsubstituted ring;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
R¹ is alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R⁵, R⁶, R⁷, R⁸, R⁹, R¹⁰, R¹¹, R¹², R¹³, R¹⁴, R¹, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

In some embodiments, J is substituted or unsubstituted arylene, heteroarylene, heterocyclylene, cycloalkylene. In some embodiments, J is a 6-carbon monocyclic or 10-carbon bicyclic aromatic ring system wherein 0, 1, 2, 3, or 4 atoms of each ring are optionally substituted. In some embodiments, A is naphthyl. In some embodiments, J is indazolyl.

In some embodiments, J is substituted arylene. In some embodiments, J is substituted phenylene. In some embodiments, J is phenylene substituted with alkyl, cycloalkyl, alkoxy, an amine group, a carboxyl group, a carboxylic acid group, a carbamide group, or an amide group, each of which is substituted or unsubstituted; cyano, halogen, or hydrogen. In some embodiments, J is phenyl substituted with alkyl, wherein alkyl is substituted. In some embodiments, J is phenylene substituted with alkyl, wherein alkyl is substituted with an amino group that is substituted or unsubstituted. In some embodiments, J is phenylene substituted with an amine group that is substituted or unsubstituted. In some embodiments, J is phenylene substituted with a carboxyl group that is substituted or unsubstituted. In some embodiments, J is phenylene substituted with cyano. In some embodiments, J is phenylene substituted with halo-.

In some embodiments, J is substituted or unsubstituted heterocyclylene. In some embodiments, J is substituted heterocyclylene.

In some embodiments, J is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, J is an aromatic 8-membered, 9-membered, 10-membered, 11-membered, or 12-membered bicyclic ring system comprising 1, 2, 3, 4, 5, or 6 heteroatoms, wherein each heteroatom is independently selected from O, N, or S. In some embodiments, J is an aromatic 5-membered, 6-membered, 7-membered, or 8-membered monocyclic ring system comprising 1, 2, or 3 heteroatoms, and the aromatic 5-membered, 6-membered, 7-membered, or 8-membered monocyclic ring system is substituted. In some embodiments, J is an 8-membered, 9-membered, 10-membered, 11-membered, or 12-membered bicyclic ring system having 1, 2, 3, 4, 5, or 6 heteroatoms, and the 8-membered, 9-membered, 10-membered, 11-membered, or 12-membered bicyclic ring system is substituted.

In some embodiments, J is pyridinyl, pyrimidinyl, thiadiazolyl, thiazolyl, pyrazolyl, thiophenyl, or oxadiazolyl, each of which is independently substituted or unsubstituted. In some embodiments, J is 1,3,5-thiadiazol-2-yl. In some embodiments, J is 1,3,4-oxadiazol-2-yl or 1,2,4-oxadiazol-2-yl. In some embodiments, J is 1,3,4-oxadiazol-2-yl.

In some embodiments, m is 1. In some embodiments, m is 2. In some embodiments, W is -Q¹-N(R³)R⁴. In some embodiments, Q¹ is alkylene, alkenylene, or alkynylene, each of which is independently substituted or unsubstituted, or a bond. In some embodiments, Q¹ is a bond.

In some embodiments, R² is hydrogen. In some embodiments, R² is substituted or unsubstituted alkyl. In some embodiments, R² is trifluoroethyl. In some embodiments, R² is cycloalkyl.

In some embodiments, R¹ is alkyl, alkoxy, aryl, heteroaryl, or heterocyclyl, each of which is independently substituted or unsubstituted; or —C(O)R¹⁶, —C(O)OR¹⁶, —C(O)NR¹⁶R¹⁷, —OR¹⁶, —NR¹⁶R¹⁷, —NR¹⁶C(O)R¹⁶, —OC(O)R¹⁶, cyano, halo, or halogen. In some embodiments, R¹ is —NR¹⁶R¹⁷. In some embodiments, R¹ is substituted alkyl.

In some embodiments, each R³ and R⁴ is independently aryl, heteroaryl, or heterocyclyl, each of which is independently substituted or unsubstituted; or hydrogen. In some embodiments, R³ is hydrogen, and R⁴ is heterocyclyl substituted at least with halo-. In some embodiments, R⁴ is heterocyclyl substituted with fluoro. In some embodiments, R⁴ is heterocyclyl substituted with chloro.

In some embodiments, R¹³ is alkyl, alkenyl, hydrogen, or halogen. In some embodiments, R¹³ is hydrogen.

In some embodiments, the compound has the formula:

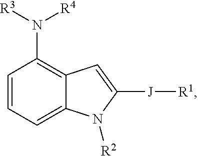

or a pharmaceutically-acceptable salt thereof, wherein the variables are as defined above.

In some embodiments, the compound has the formula:

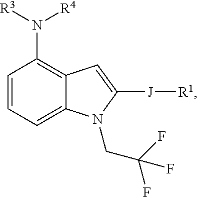

or a pharmaceutically-acceptable salt thereof, wherein the variables are as defined above.

In some embodiments, the compound has the formula:

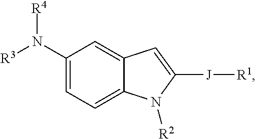

or a pharmaceutically-acceptable salt thereof, wherein the variables are as defined above.

In some embodiments, the compound has the formula:

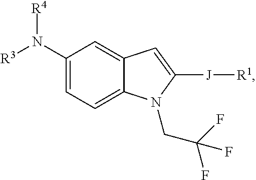

or a pharmaceutically-acceptable salt thereof, wherein the variables are as defined above.

In some embodiments, the disclosure provides a compound of the formula:

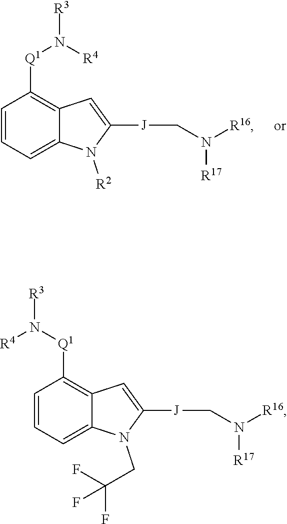

or a pharmaceutically-acceptable salt thereof, wherein the variables are as defined above.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

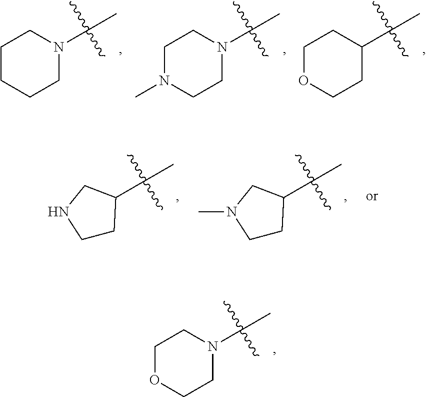

wherein the ring is substituted or unsubstituted. In some embodiments, R³ is H, and R⁴ is a ring that is

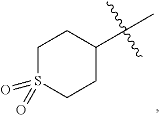 [SMILES: *C1CCS(=O)(=O)CC1]

wherein the ring is substituted or unsubstituted. In some embodiments, R³ is H, and R⁴ is a ring that is

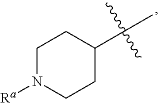

wherein the ring is substituted or unsubstituted. In some embodiments, Ra is alkylene. In some embodiments, Ra is methyl. In some embodiments, R³ is H, and R⁴ is a ring that is

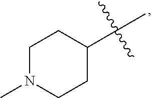 [SMILES: *C1CCN(C)CC1]

wherein the ring is substituted or unsubstituted. In some embodiments, R³ is H, and R⁴ is a ring that is

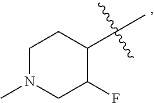 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

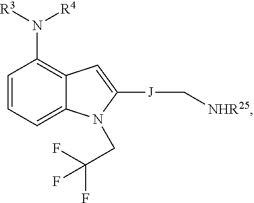

wherein R²⁵ is alkyl, alkenyl, alkynyl, aryl, heteroaryl, or heterocyclyl, each of which is independently substituted or unsubstituted, or —C(O)R¹⁶, —C(O)NR¹⁶R¹⁷, or hydrogen. In some embodiments, R²⁵ is aryl that is substituted or unsubstituted. In some embodiments, R²⁵ is substituted phenyl. In some embodiments, R²⁵ is —C(O)R¹⁶, wherein R¹⁶ is alkyl, aryl, heteroaryl, or heterocyclyl. In some embodiments, R²⁵ is —C(O)R¹⁶, wherein R¹⁶ is substituted phenyl.

In some embodiments, the disclosure provides a compound of the formula:

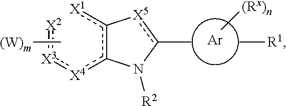

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each W is independently -Q¹-N(R³)R⁴, -Q¹-OR⁴, or -Q¹-R⁴;
   wherein at least one of X¹, X², X³, and X⁴ is a carbon atom connected to Q¹;
Ar is unsubstituted or substituted arylene;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
n is 0, 1, 2, 3, or 4;
each R^{x} and R¹ is independently alkyl, alkenyl, alkynyl, alkoxy, aryl, heteroaryl, or heterocyclyl, each of which is independently substituted or unsubstituted; or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, cyano, halo, or hydrogen; or R¹ and R^{x} together with Ar form a fused ring;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R⁵, R⁶, R⁷, R⁸, R⁹, R¹⁰, R, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

The pattern of dashed bonds can be chosen to provide an aromatic system, for example, an indole, a pyrrolopyridine, a pyrrolopyrimidine, or a pyrrolopyrazine. In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Ar is a 6-carbon monocyclic or 10-carbon bicyclic aromatic ring system wherein 0, 1, 2, 3, or 4 atoms of each ring are optionally substituted. In some embodiments, Ar is phenylene. In some embodiments, Ar is naphthylene. In some embodiments, Ar is indazolylene.

R¹ can be alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, or aryl, each of which is independently substituted or unsubstituted; or —NR²¹R²², halo, or hydrogen. In some embodiments, R¹ is methyl, cyclohexyl, methylene, methoxy, or benzyl. In some embodiments, R¹ is fluoro or chloro. In some embodiments, R¹ is phenyl. In some embodiments, R¹ is hydrogen.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is alkyl substituted with —C(O)NR¹⁶R¹⁷. In some embodiments, R¹ is methyl substituted with —C(O)NR¹⁶R¹⁷. In some embodiments, R¹ is alkyl substituted with —C(O)OR¹⁶. In some embodiments, R¹ is methyl substituted with COOH.

In some embodiments, m is 1, 2, 3, or 4. In some embodiments, m is 1. In some embodiments, X³ is carbon atom connected to Q¹, and m is 1. In some embodiments, n is 1, 2, or 3. In some embodiments, n is 1. In some embodiments, n is 2. In some embodiments, n is 0.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R2 is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen, and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

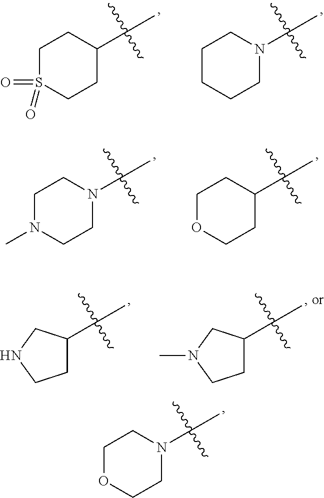

wherein the ring is substituted or unsubstituted. In some embodiments, R³ is H, and R⁴ is a ring that is

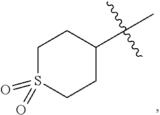 [SMILES: *C1CCS(=O)(=O)CC1]

wherein the ring is substituted or unsubstituted. In some embodiments, R³ is H, and R⁴ is a ring that is

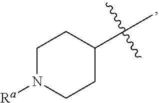

wherein the ring is substituted or unsubstituted. In some embodiments, R^{a} is alkylene. In some embodiments, R^{a} is methyl. In some embodiments, R³ is H, and R⁴ is a ring that is

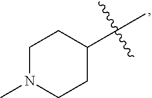 [SMILES: *C1CCN(C)CC1]

wherein the ring is substituted or unsubstituted. In some embodiments, R³ is H, and R⁴ is a ring that is

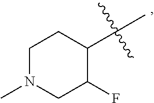 [SMILES: *C1CCN(C)CC1F]

wherein the ring is substituted or unsubstituted. In some embodiments, R³ is H, and R⁴ is a ring that is

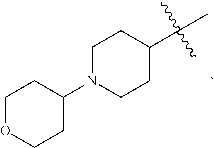 [SMILES: *C1CCN(C2CCOCC2)CC1]

In some embodiments, the disclosure provides a compound of the formula:

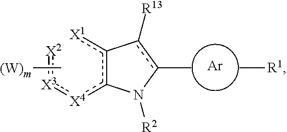

wherein the variables are as defined above.

In some embodiments, the disclosure provides a compound of the formula:

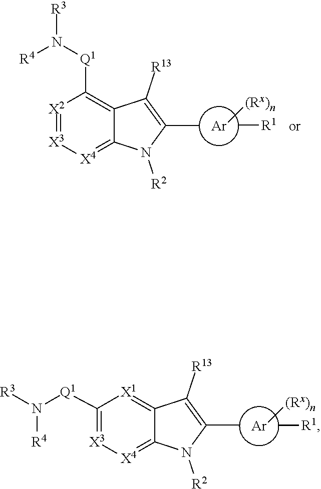

wherein:
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
Ar is unsubstituted or substituted aryl;
Q¹ is C═O, C═S, C═CR¹⁴R¹⁵, C═NR¹⁴, alkylene, alkenylene, or alkynylene, each of which is independently substituted or unsubstituted, or a bond;
n is 0, 1, 2, 3, or 4;
each R^{X} and R¹ is independently alkyl, alkenyl, alkynyl, alkoxy, aryl, heteroaryl, or heterocyclyl, each of which is independently substituted or unsubstituted; or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, cyano, halo, or hydrogen; or R¹ and R^{X} together with Ar form a fused ring;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

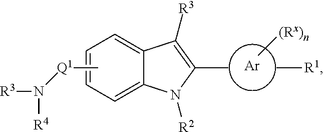

wherein the variables are as defined above.

In some embodiments, Ar is a 6-carbon monocyclic or 10-carbon bicyclic aromatic ring system wherein 0, 1, 2, 3, or 4 atoms of each ring are optionally substituted. In some embodiments, Ar is phenylene. In some embodiments, Ar is naphthylene. In some embodiments, Ar is indazolylene.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is alkyl substituted with —C(O)NR¹⁶R¹⁷. In some embodiments, R¹ is methyl substituted with —C(O)NR¹⁶R¹⁷. In some embodiments, R¹ is alkyl substituted with —C(O)OR¹⁶. In some embodiments, R¹ is methyl substituted with COOH.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R2 is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen, and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

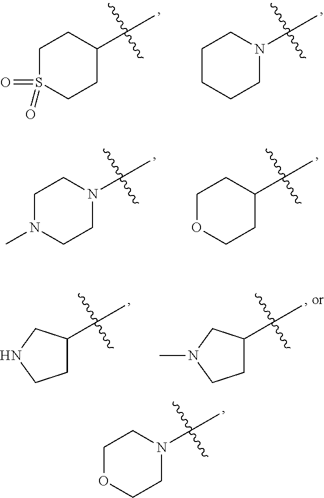

wherein the ring is substituted or unsubstituted. In some embodiments, R³ is H, and R⁴ is a ring that is

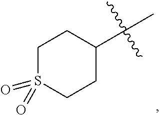 [SMILES: *C1CCS(=O)(=O)CC1]

wherein the ring is substituted or unsubstituted. In some embodiments, R³ is H, and R⁴ is a ring that is

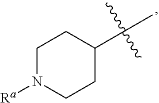

wherein the ring is substituted or unsubstituted. In some embodiments, R^{a} is alkylene. In some embodiments, R^{a} is methyl. In some embodiments, R³ is H, and R⁴ is a ring that is

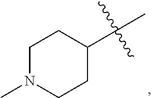 [SMILES: *C1CCN(C)CC1]

wherein the ring is substituted or unsubstituted. In some embodiments, R³ is H, and R⁴ is a ring that is

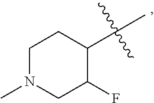 [SMILES: *C1CCN(C)CC1F]

wherein the ring is substituted or unsubstituted. In some embodiments, R³ is H, and R⁴ is a ring that is

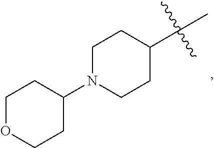 [SMILES: *C1CCN(C2CCOCC2)CC1]

In some embodiments, the disclosure provides a compound of the formula:

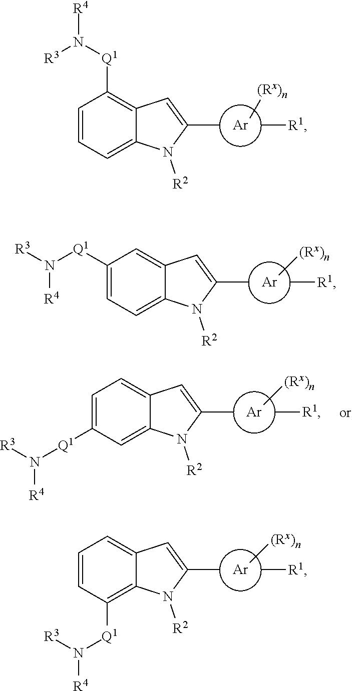

or a pharmaceutically-acceptable salt thereof, wherein the variables are as defined above.

In some embodiments, the disclosure provides a compound of the formula:

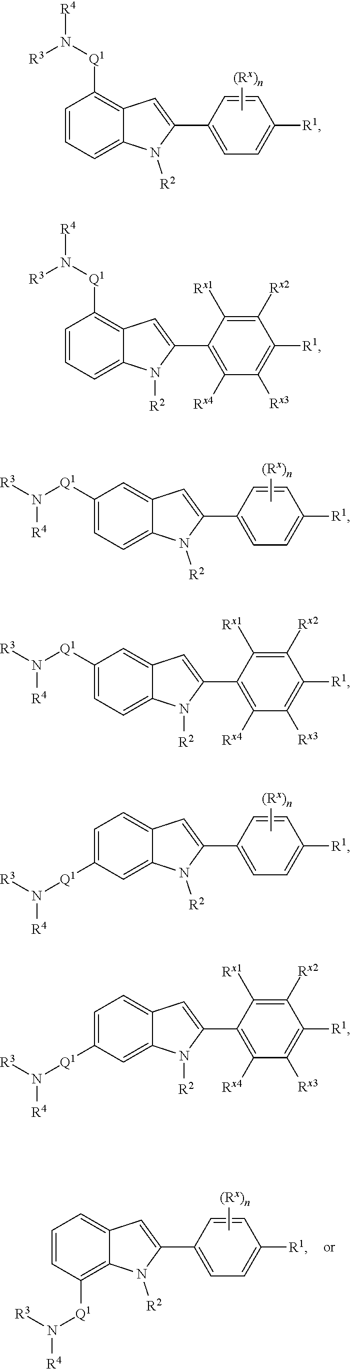

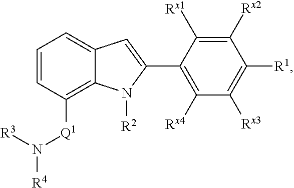

wherein:
Q¹ is alkylene, alkenylene, or alkynylene, each of which is independently substituted or unsubstituted, or C═O, C═S, C═CR¹⁴R¹⁵, C═NR¹⁴, or a bond;
each R¹, R^{x}, R^{x1}, R^{x2}, R^{x3}, and R^{x4} is independently alkyl, alkenyl, alkynyl, alkoxy, aryl, heteroaryl, or heterocyclyl, each of which is independently substituted or unsubstituted; or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, cyano, halo, or hydrogen; or R¹ and R^{x} together with Ar form a fused ring;
each R³ and R⁴ is independently alkyl, alkenyl, alkynyl, aryl, heteroaryl, or heterocyclyl, each of which is independently substituted or unsubstituted, or hydrogen, or —C(O)R¹⁹, —C(O)OR¹⁹, —C(O)NR¹⁹R²⁰, —SOR¹⁹, or —SO₂R¹⁹; or R³ and R⁴ together with the nitrogen atom to which R³ and R⁴ are bound form a ring, wherein the ring is substituted or unsubstituted;
n is 0, 1, 2, 3, or 4;
each R², R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is methyl substituted with NR¹⁶R¹⁷, wherein R¹⁶ is hydrogen, and R¹⁷ is alkyl, aryl, heteroaryl, an amino group, a carboxyl group, or an ester group, any of which is substituted or unsubstituted. In some embodiments, R¹ is methyl substituted with NR¹⁶R¹⁷, wherein R¹⁶ is hydrogen, and R¹⁷ is substituted or unsubstituted alkyl, aryl, or heteroaryl. In some embodiments, R¹ is methyl substituted with NR¹⁶R¹⁷, wherein R¹⁶ is hydrogen, and R¹⁷ is substituted or unsubstituted phenyl. In some embodiments, R¹ is methyl substituted with NR¹⁶R¹⁷, wherein R¹⁶ is hydrogen, and R¹⁷ is substituted or unsubstituted pyridinyl.

In some embodiments, R¹ is —C(O)NR¹⁶R¹⁷. In some embodiments, R¹ is —C(O)NR¹⁶R¹⁷, wherein R¹⁶ and R¹⁷ are hydrogen. In some embodiments, R¹ is —C(O)NR¹⁶R¹⁷, wherein R¹⁶ is hydrogen, and R¹⁷ alkyl. In some embodiments, R¹ is —C(O)NR¹⁶R¹⁷, wherein R¹⁶ is hydrogen, and R¹⁷ methyl. In some embodiments, R¹ is —C(O)OR¹⁶. In some embodiments, R¹ is —C(O)OH. In some embodiments, R¹ is methyl. In some embodiments, R¹ is halogen. In some embodiments, R¹ is chloro or fluoro.

In some embodiments, n is 0, 1, 2, or 3. In some embodiments, n is 1. In some embodiments, n is 2. In some embodiments, n is 0.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen, and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

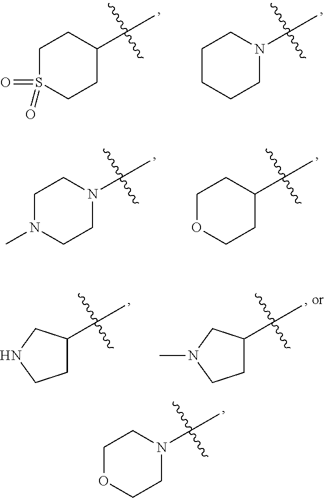

wherein the ring is substituted or unsubstituted. In some embodiments, R³ is H, and R⁴ is a ring that is

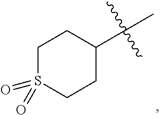 [SMILES: *C1CCS(=O)(=O)CC1]

wherein the ring is substituted or unsubstituted. In some embodiments, R³ is H, and R⁴ is a ring that is

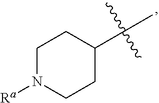

wherein the ring is substituted or unsubstituted. In some embodiments, R^{a} is alkylene. In some embodiments, R^{a} is methyl. In some embodiments, R³ is H, and R⁴ is a ring that is

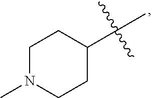 [SMILES: *C1CCN(C)CC1]

wherein the ring is substituted or unsubstituted. In some embodiments, R³ is H, and R⁴ is a ring that is

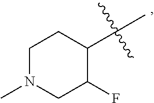 [SMILES: *C1CCN(C)CC1F]

wherein the ring is substituted or unsubstituted. In some embodiments, R³ is H, and R⁴ is a ring that is

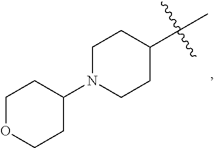 [SMILES: *C1CCN(C2CCOCC2)CC1]

In some embodiments, the disclosure provides a compound of the formula:

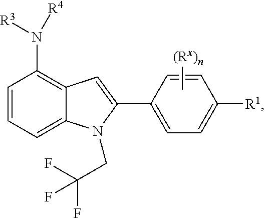

or a pharmaceutically-acceptable salt thereof, wherein the variables are as defined above.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is methyl substituted with NR¹⁶R¹⁷, wherein R¹⁶ is hydrogen, and R¹⁷ is alkyl, aryl, heteroaryl, an amino group, a carboxyl group, or an ester group, any of which is substituted or unsubstituted. In some embodiments, R¹ is methyl substituted with NR¹⁶R¹⁷, wherein R¹⁶ is hydrogen, and R¹⁷ is substituted or unsubstituted alkyl, aryl, or heteroaryl. In some embodiments, R¹ is methyl substituted with NR¹⁶R¹⁷, wherein R¹⁶ is hydrogen, and R¹⁷ is substituted or unsubstituted phenyl. In some embodiments, R¹ is methyl substituted with NR¹⁶R¹⁷, wherein R¹⁶ is hydrogen, and R¹⁷ is substituted or unsubstituted pyridinyl.

In some embodiments, R¹ is —C(O)NR¹⁶R¹⁷. In some embodiments, R¹ is —C(O)NR¹⁶R¹⁷, wherein R¹⁶ and R¹⁷ are hydrogen. In some embodiments, R¹ is —C(O)NR¹⁶R¹⁷, wherein R¹⁶ is hydrogen, and R¹⁷ alkyl. In some embodiments, R¹ is —C(O)NR¹⁶R¹⁷, wherein R¹⁶ is hydrogen, and R¹⁷ methyl. In some embodiments, R¹ is —C(O)OR¹⁶. In some embodiments, R¹ is —C(O)OH. In some embodiments, R¹ is methyl. In some embodiments, R¹ is halogen. In some embodiments, R¹ is chloro or fluoro.

In some embodiments, n is 1, 2, or 3. In some embodiments, n is 1. In some embodiments, n is 2. In some embodiments, n is 0.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen, and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R³ is H, and R⁴ is a ring that is:

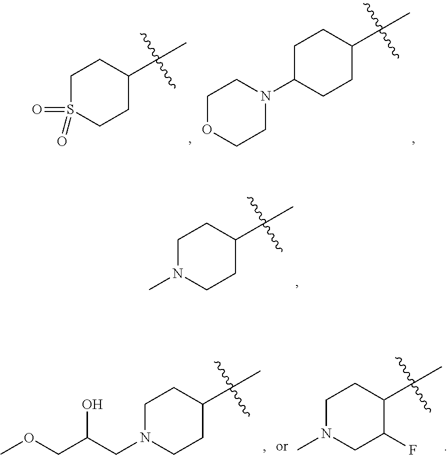

In some embodiments, R³ is H, and R⁴ is a ring that is

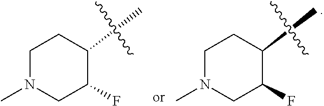

In some embodiments, R³ is H, and R⁴ is a ring that is

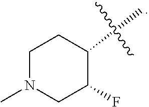

Non-limiting examples of compounds of the disclosure include compounds of any of the following formulae:

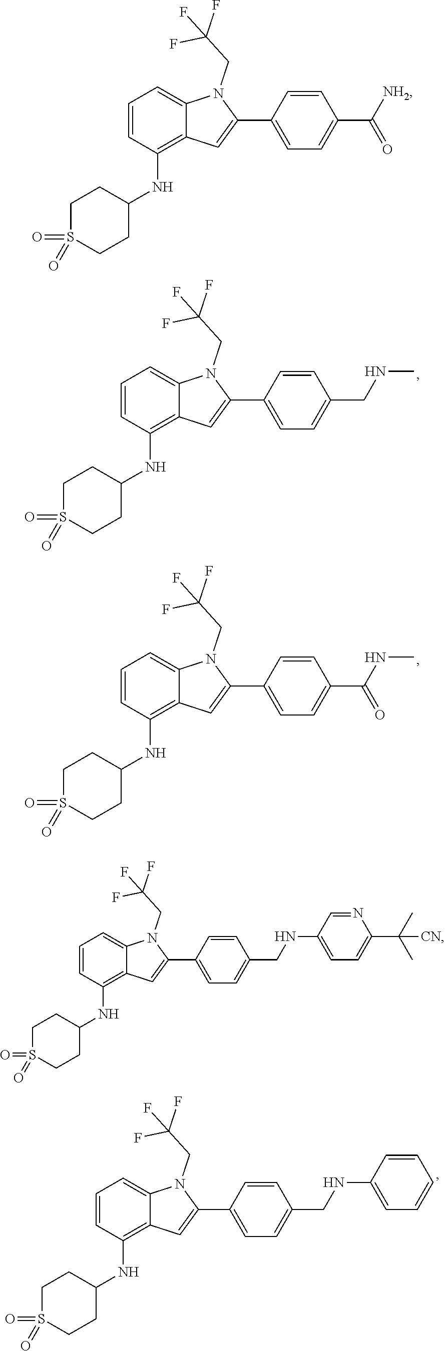 [SMILES: NC(=O)c1ccc(-c2cc3c(NC4CCS(=O)(=O)CC4)cccc3n2CC(F)(F)F)cc1 ; CNCc1ccc(-c2cc3c(NC4CCS(=O)(=O)CC4)cccc3n2CC(F)(F)F)cc1 ; CNC(=O)c1ccc(-c2cc3c(NC4CCS(=O)(=O)CC4)cccc3n2CC(F)(F)F)cc1 ; CC(C)(C#N)c1ccc(NCc2ccc(-c3cc4c(NC5CCS(=O)(=O)CC5)cccc4n3CC(F)(F)F)cc2)cn1 ; O=S1(=O)CCC(Nc2cccc3c2cc(-c2ccc(CNc4ccccc4)cc2)n3CC(F)(F)F)CC1]

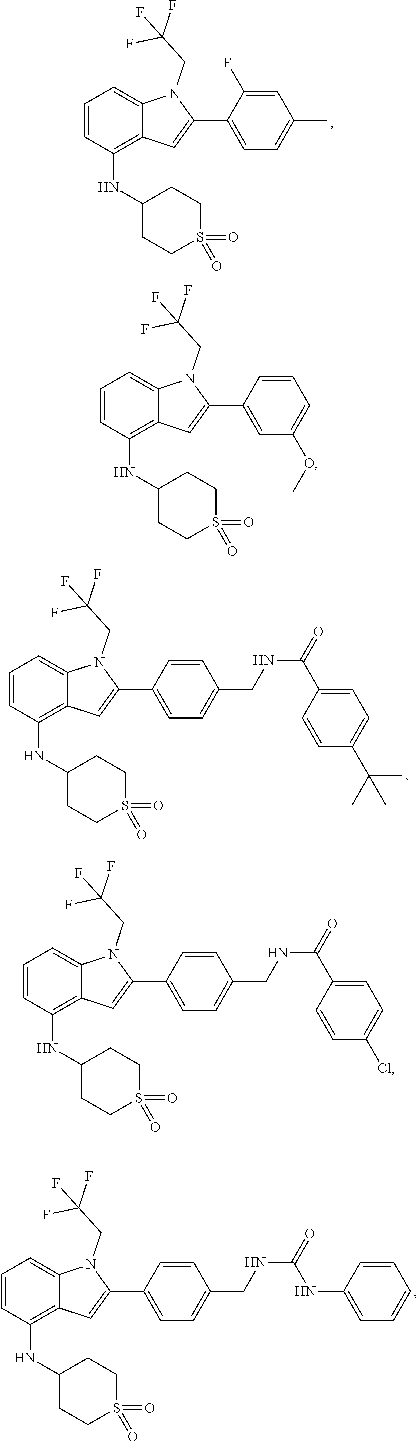

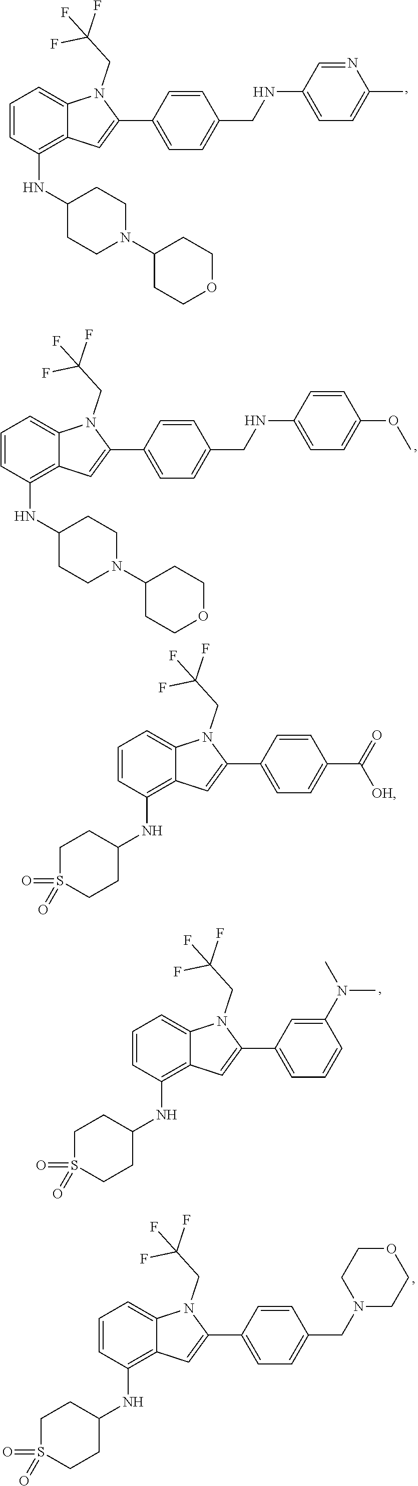

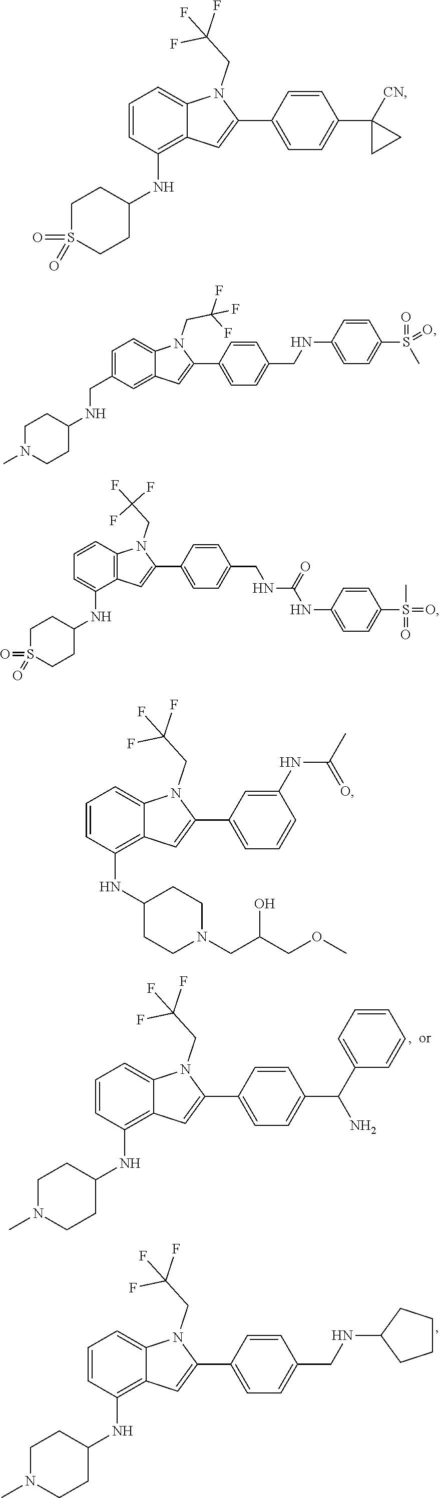

or a pharmaceutically-acceptable salt thereof.

In some embodiments, the disclosure provides a compound of the formula:

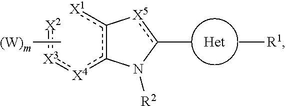

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each W is independently -Q¹-N(R³)R⁴, -Q¹-OR⁴, or -Q¹-R⁴;
   wherein at least one of X¹, X², X³, and X⁴ is a carbon atom connected to Q¹;
Het is substituted or unsubstituted heteroarylene;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
R¹ is alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R7R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R⁵, R⁶, R⁷, R⁸, R⁹, R¹⁰, R, R¹², R¹³, R¹⁴, R, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

The pattern of dashed bonds can be chosen to provide an aromatic system, for example, an indole, a pyrrolopyridine, a pyrrolopyrimidine, or a pyrrolopyrazine. In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Het is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, Het is an aromatic 8-membered, 9-membered, 10-membered, 11-membered, or 12-membered bicyclic ring system comprising 1, 2, 3, 4, 5, or 6 heteroatoms, wherein each heteroatom is independently selected from O, N, or S. In some embodiments, Het is an aromatic 5-membered, 6-membered, 7-membered, or 8-membered monocyclic ring system comprising 1, 2, or 3 heteroatoms, and the aromatic 5-membered, 6-membered, 7-membered, or 8-membered monocyclic ring system is substituted. In some embodiments, Het is an 8-membered, 9-membered, 10-membered, 11-membered, or 12-membered bicyclic ring system having 1, 2, 3, 4, 5, or 6 heteroatoms, and the 8-membered, 9-membered, 10-membered, 11-membered, or 12-membered bicyclic ring system is substituted.

In some embodiments, Het is pyridinyl, pyrimidinyl, thiadiazolyl, thiazolyl, pyrazolyl, thiophenyl, or oxadiazolyl, each of which is independently substituted or unsubstituted. In some embodiments, Het is 1,3,5-thiadiazol-2-yl. In some embodiments, Het is 1,3,4-oxadiazol-2-yl or 1,2,4-oxadiazol-2-yl. In some embodiments, Het is 1,3,4-oxadiazol-2-yl.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, —NR²¹R²², or aryl, each of which is independently substituted or unsubstituted; halo or hydrogen. In some embodiments, R¹ is methyl, cyclohexyl, methylene, methoxy, or benzyl. In some embodiments, R¹ is fluoro or chloro. In some embodiments, R¹ is phenyl. In some embodiments, R¹ is hydrogen.

In some embodiments, R¹ is a substituted alkyl or alkylene. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is substituted alkyl. In some embodiments, R¹ is alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, m is 1, 2, 3, or 4. In some embodiments, m is 1. In some embodiments, X¹ is carbon atom connected to Q¹, and m is 1. In some embodiments, X² is carbon atom connected to Q¹, and m is 1.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, Q¹ is C₁-alkylene, R¹⁶ is aryl, and R¹⁷ is alkyl. In some embodiments, Q¹ is C₁-alkylene, R¹⁶ is aryl, and R¹⁷ is hydrogen. In some embodiments, Q¹ is C₁-alkylene, R¹⁶ is heteroaryl, and R¹⁷ is alkyl. In some embodiments, Q¹ is C₁-alkylene, R¹⁶ is heteroaryl, and R¹⁷ is hydrogen. In some embodiments, Q¹ is C₁-alkylene, R¹⁶ is substituted heteroaryl, and R¹⁷ is hydrogen. In some embodiments, Q¹ is C₁-alkylene, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano.

In some embodiments, R² is hydrogen or alkyl. In some embodiments, R² is substituted alkyl. In some embodiments, R² is trifluoroethyl. In some embodiments, R¹³ is alkyl, alkenyl, hydrogen, or halogen. In some embodiments, R¹³ is methyl, ethyl, propyl, iso-propyl, butyl or tert-butyl. In some embodiments, R² is trifluoroethyl, and R¹³ is hydrogen.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen; and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

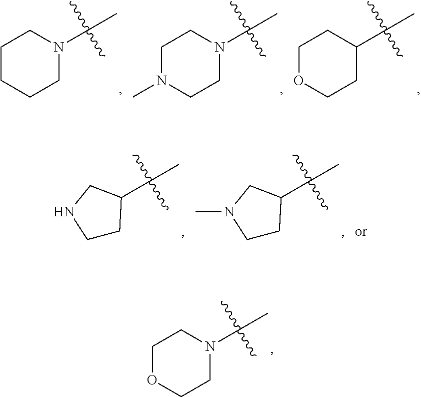

wherein the ring is substituted or unsubstituted. In some embodiments, R³ is H, and R⁴ is a ring that is

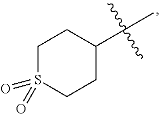 [SMILES: *C1CCS(=O)(=O)CC1]

wherein the ring is substituted or unsubstituted. In some embodiments, R³ is H, and R⁴ is a ring that is

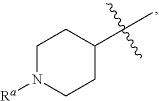

wherein the ring is substituted or unsubstituted. In some embodiments, R^{a} is alkylene. In some embodiments, R^{a} is methyl. In some embodiments, R³ is H, and R⁴ is a ring that is

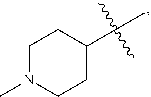 [SMILES: *C1CCN(C)CC1]

wherein the ring is substituted or unsubstituted. In some embodiments, R³ is H, and R⁴ is a ring that is

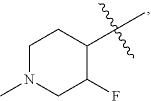 [SMILES: *C1CCN(C)CC1F]

wherein the ring is substituted or unsubstituted.

In some embodiments, R³ and R⁴ together with the nitrogen atom to which R³ and R⁴ are bound form a ring, wherein the ring is substituted or unsubstituted. In some embodiments, R³ and R⁴ together with the nitrogen atom to which R³ and R⁴ are bound form a substituted heterocycle. In some embodiments, R³ and R⁴ together with the nitrogen atom to which R³ and R⁴ are bound form a heterocycle substituted with a hydroxyl group, halogen, amino group, or alkyl group. In some embodiments, R³ and R⁴ together with the nitrogen atom to which R³ and R⁴ are bound form a heterocycle, wherein the heterocycle is substituted by a substituted or unsubstituted heterocycle.

In some embodiments, the disclosure provides a compound of the formula:

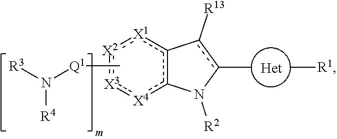

or a pharmaceutically-acceptable salt thereof, wherein the variables are as defined above.

In some embodiments, the disclosure provides a compound of the formula:

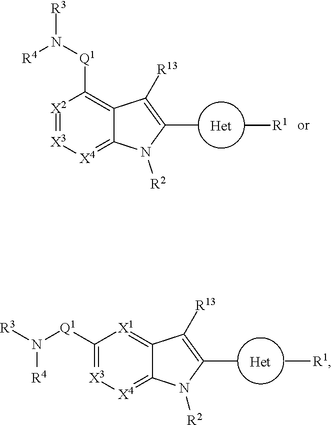

or a pharmaceutically-acceptable salt thereof, wherein the variables are as defined above.

In some embodiments, the compound is of the formula:

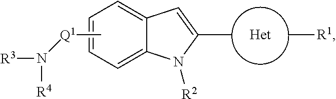

or a pharmaceutically-acceptable salt thereof, wherein the variables are as defined above.

In some embodiments, the disclosure provides a compound of the formula:

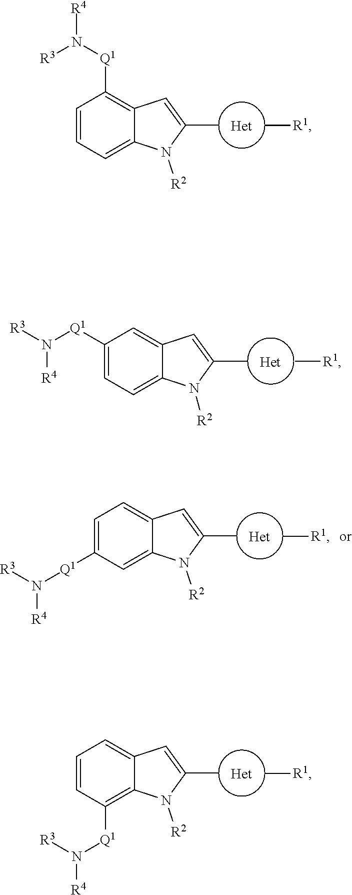

or a pharmaceutically-acceptable salt thereof, wherein the variables are as defined above.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, or aryl, each of which is independently substituted or unsubstituted; or —NR²¹R²², halo, or hydrogen.

In some embodiments, R¹ is substituted alkyl. In some embodiments, R¹ is alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, R² is hydrogen or alkyl. In some embodiments, R² is substituted alkyl. In some embodiments, R² is trifluoroethyl.

In some embodiments, Q¹ is alkylene, alkenylene, or alkynylene, each of which is independently substituted or unsubstituted, or C═O, C═S, C═CR¹⁴R⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

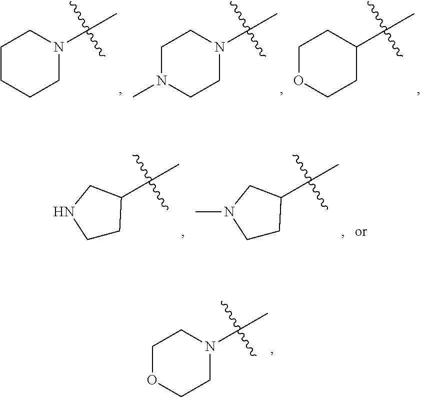

wherein the ring is substituted or unsubstituted. In some embodiments, R³ is H, and R⁴ is a ring that is

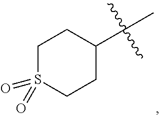 [SMILES: *C1CCS(=O)(=O)CC1]

wherein the ring is substituted or unsubstituted. In some embodiments, R³ is H, and R⁴ is a ring that is

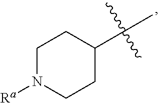

wherein the ring is substituted or unsubstituted. In some embodiments, R^{a} is alkylene. In some embodiments, R^{a} is methyl. In some embodiments, R³ is H, and R⁴ is a ring that is

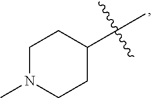 [SMILES: *C1CCN(C)CC1]

wherein the ring is substituted or unsubstituted. In some embodiments, R³ is H, and R⁴ is a ring that is

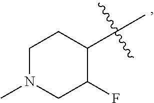 [SMILES: *C1CCN(C)CC1F]

wherein the ring is substituted or unsubstituted.

In some embodiments, the disclosure provides a compound of the formula:

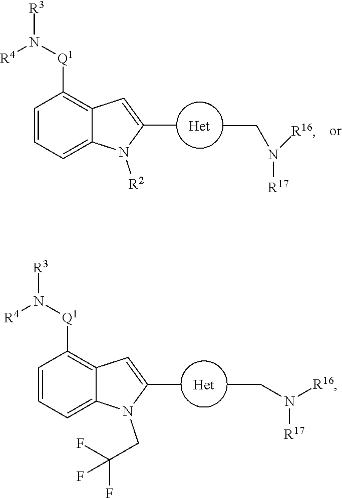

or a pharmaceutically-acceptable salt thereof, wherein the variables are as defined above.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

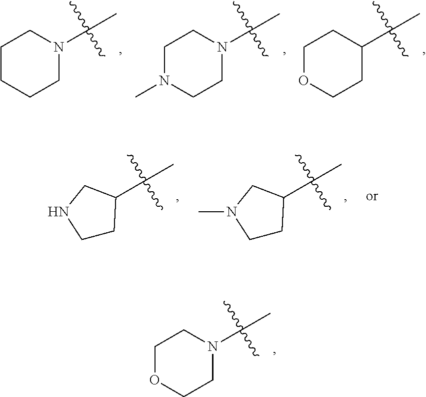

wherein the ring is substituted or unsubstituted. In some embodiments, R³ is H, and R⁴ is a ring that is

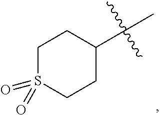 [SMILES: *C1CCS(=O)(=O)CC1]

wherein the ring is substituted or unsubstituted. In some embodiments, R³ is H, and R⁴ is a ring that is

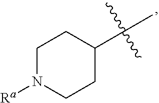

wherein the ring is substituted or unsubstituted. In some embodiments, R^{a} is alkylene. In some embodiments, R^{a} is methyl. In some embodiments, R³ is H, and R⁴ is a ring that is

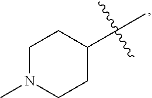 [SMILES: *C1CCN(C)CC1]

wherein the ring is substituted or unsubstituted. In some embodiments, R³ is H, and R⁴ is a ring that is

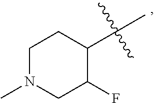 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

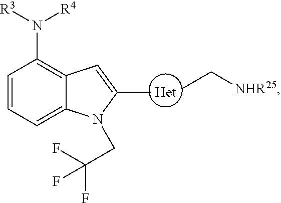

wherein R²⁵ is alkyl, alkenyl, alkynyl, aryl, heteroaryl, or heterocyclyl, each of which is independently substituted or unsubstituted, or —C(O)R¹⁶, —C(O)NR¹⁶R¹⁷, or hydrogen. In some embodiments, R²⁵ is aryl that is substituted or unsubstituted. In some embodiments, R²⁵ is substituted phenyl. In some embodiments, R²⁵ is —C(O)R¹⁶, wherein R¹⁶ is alkyl, aryl, heteroaryl, or heterocyclyl. In some embodiments, R²⁵ is —C(O)R¹⁶, wherein R¹⁶ is substituted phenyl; or a pharmaceutically-acceptable salt thereof,
In some embodiments, the compound is of the formula:

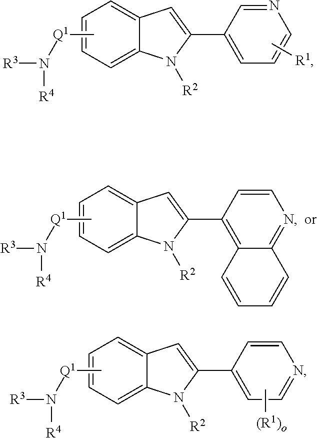

wherein:
Q¹ is alkylene, alkenylene, or alkynylene, each of which is independently substituted or unsubstituted, or C═O, C═S, C═CR¹⁴R¹⁵, C═NR¹⁴, or a bond;
o is 1, 2, 3, or 4.
R¹ is alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

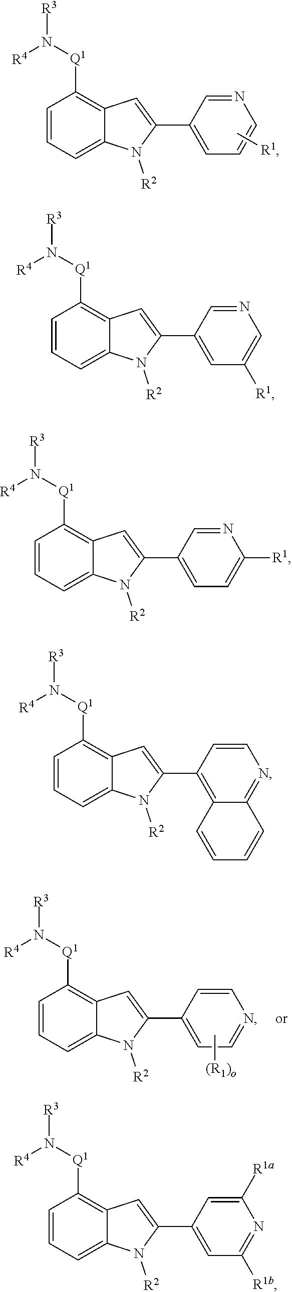

wherein:
Q¹ is alkylene, alkenylene, or alkynylene, each of which is independently substituted or unsubstituted, or C═O, C═S, C═CR¹⁴R¹⁵, C═NR¹⁴, or a bond;
each R¹, R^{1a}, and R^{1b} is independently alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
o is 0, 1, 2, 3, or 4;
each R², R¹⁴, R¹⁵, R¹, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, each R^{1a} and R^{1b} is independently alkyl, alkoxy, aryl, heteroaryl, heterocyclyl, or NR¹⁶R¹⁷. In some embodiments, R^{1a} is unsubstituted phenyl, and R^{1b} is amino.

In some embodiments, the compound is of the formula:

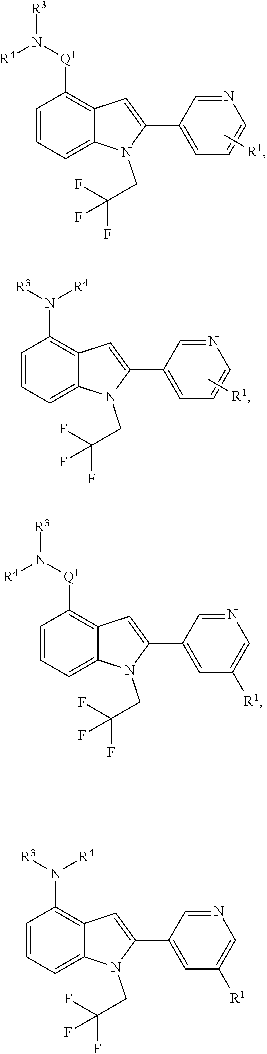

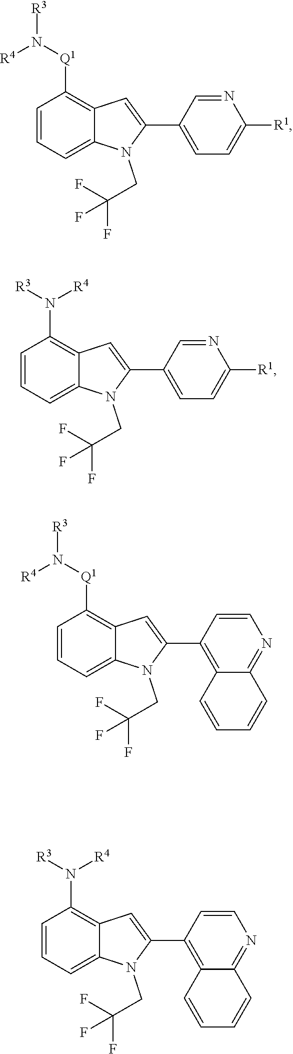

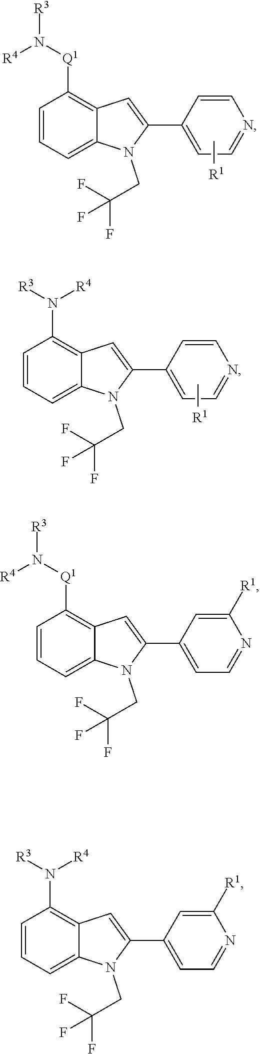

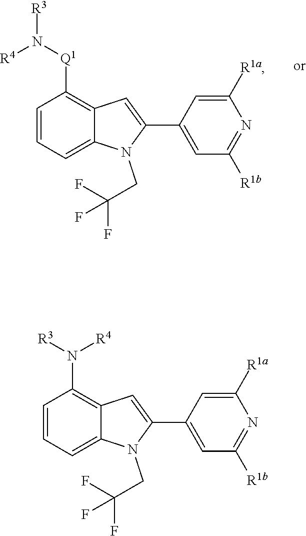

or a pharmaceutically-acceptable salt thereof, wherein the variables are as defined above.

In some embodiments, R¹ is alkyl, alkenyl, alkynyl, alkoxy, aryl, heteroaryl, heterocyclyl, or halo, each of which is independently substituted or unsubstituted, or —C(O)NR¹⁶R¹⁷ or hydrogen. In some embodiments, R¹ is alkyl, alkoxy, aryl, or halo. In some embodiments, R¹ is methoxy, methyl, or phenyl. In some embodiments, each R^{1a} and R^{1b} is independently alkyl, alkoxy, aryl, heteroaryl, heterocyclyl, or NR¹⁶R¹⁷. In some embodiments, R^{1a} is unsubstituted phenyl, and R^{1b} is amino.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

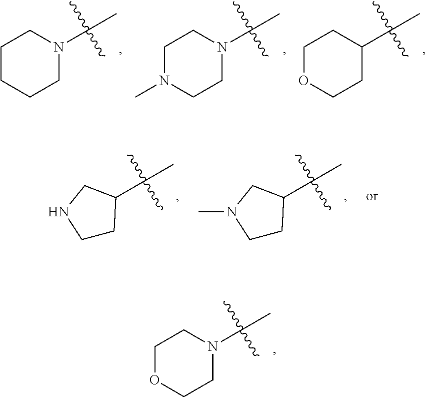

wherein the ring is substituted or unsubstituted. In some embodiments, R³ is H, and R⁴ is a ring that is

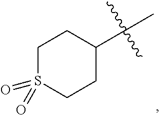 [SMILES: *C1CCS(=O)(=O)CC1]

wherein the ring is substituted or unsubstituted. In some embodiments, R³ is H, and R⁴ is a ring that is

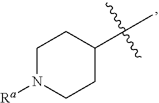

wherein the ring is substituted or unsubstituted. In some embodiments, R^{a} is alkylene. In some embodiments, R^{a} is methyl. In some embodiments, R³ is H, and R⁴ is a ring that is

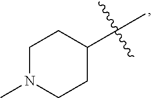 [SMILES: *C1CCN(C)CC1]

wherein the ring is substituted or unsubstituted. In some embodiments, R³ is H, and R⁴ is a ring that is

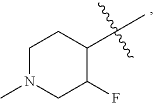 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

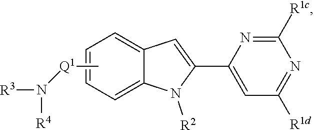

wherein:
Q¹ is alkylene, alkenylene, or alkynylene, each of which is independently substituted or unsubstituted, or C═O, C═S, C═CR¹⁴R¹⁵, C═NR¹⁴, or a bond;
each R^{1c} and R^{1d} is independently alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen, or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, each R^{1c} and R^{1d} is independently alkyl, alkenyl, alkynyl, alkoxy, aryl, heteroaryl, or heterocyclyl, each of which is independently substituted or unsubstituted, or —OR¹⁶, —NR¹⁶R¹⁷, —NR¹⁶C(O)R¹⁶, or hydrogen.

In some embodiments, the compound is of the formula:

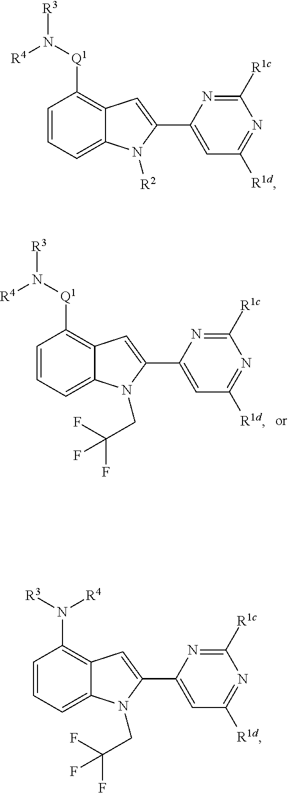

or a pharmaceutically-acceptable salt thereof, wherein the variables are as defined above.

In some embodiments, each R^{1c} and R^{1d} is independently alkyl, alkenyl, alkynyl, alkoxy, aryl, heteroaryl, or heterocyclyl, each of which is independently substituted or unsubstituted, or halogen, —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R^{1c} is amino, and R^{1d} is phenyl. In some embodiments, R^{1c} is amino, and R^{1d} is cycloalkenyl.

In some embodiments, the compound is of the formula:

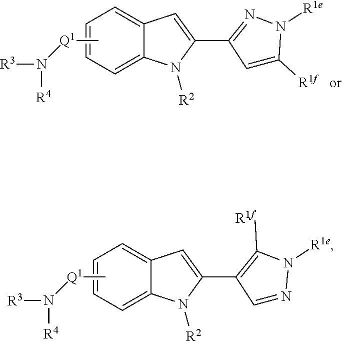

wherein:
Q¹ is alkylene, alkenylene, or alkynylene, each of which is independently substituted or unsubstituted, or C═O, C═S, C═CR¹⁴R¹⁵, C═NR¹⁴, or a bond;
each R^{1e} and R^{1f} is independently alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

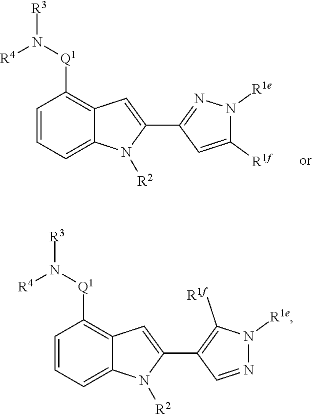

or a pharmaceutically-acceptable salt thereof, wherein the variables are as defined above.

In some embodiments, the compound is of the formula:

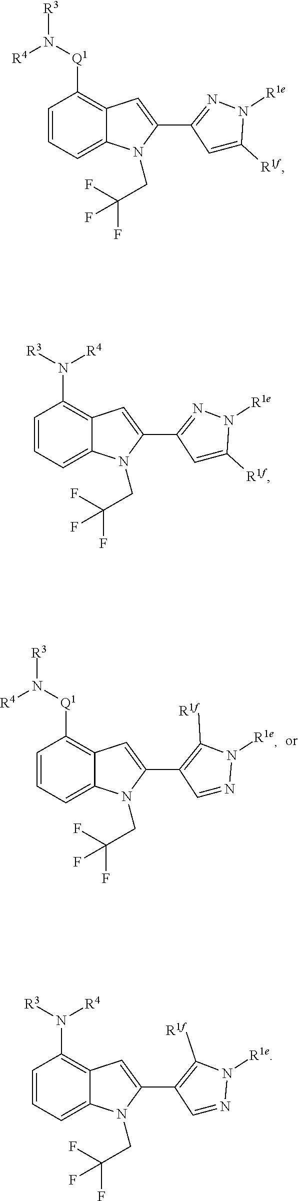

or a pharmaceutically-acceptable salt thereof, wherein the variables are as defined above.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

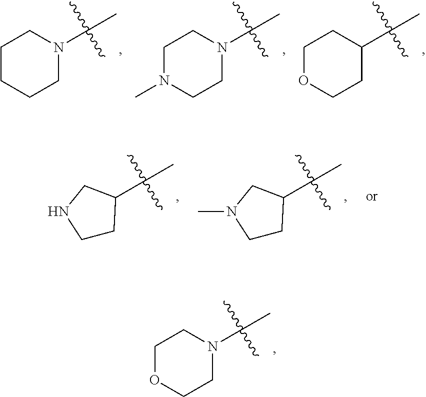

wherein the ring is substituted or unsubstituted. In some embodiments, R³ is H, and R⁴ is a ring that is

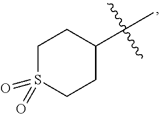 [SMILES: *C1CCS(=O)(=O)CC1]

wherein the ring is substituted or unsubstituted. In some embodiments, R³ is H, and R⁴ is a ring that is

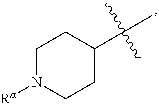

wherein the ring is substituted or unsubstituted. In some embodiments, R^{a} is alkylene. In some embodiments, R^{a} is methyl. In some embodiments, R³ is H, and R⁴ is a ring that is

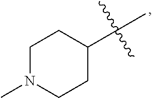 [SMILES: *C1CCN(C)CC1]

wherein the ring is substituted or unsubstituted. In some embodiments, R³ is H, and R⁴ is a ring that is

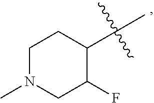 [SMILES: *C1CCN(C)CC1F]

wherein the ring is substituted or unsubstituted.

In some embodiments, each R^{1e} and R^{1f} is independently alkyl, NR¹⁶R¹⁷, aryl, heteroaryl, or heterocyclyl, each of which is independently substituted or unsubstituted, or hydrogen. In some embodiments, R^{1e} is substituted alkyl, and R^{1f} is hydrogen. In some embodiments, R^{1e} is hydrogen, and R^{1f} is NR¹⁶R¹⁷, wherein each R¹⁶ and R¹⁷ is independently alkyl, alkenyl, aryl, heteroaryl, or heterocyclyl, each of which is independently substituted or unsubstituted, or hydrogen. In some embodiments, R^{1e} is hydrogen, and R^{1f} is NR¹⁶R¹⁷, wherein R¹⁶ is hydrogen, and R¹⁷ is alkyl. In some embodiments, R^{1e} is hydrogen, and R^{1f} is NR¹⁶R¹⁷, wherein R¹⁶ is hydrogen, and R¹⁷ is phenyl. In some embodiments, R^{1e} is hydrogen, and R^{1f} is amino.

In some embodiments, the compound is of the formula:

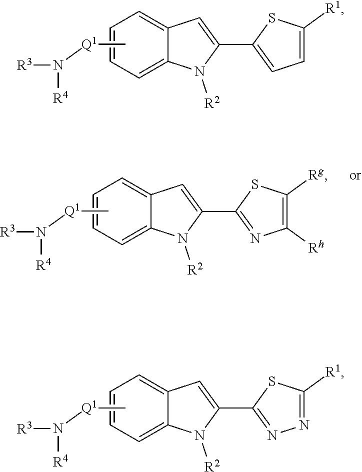

wherein:
Q¹ is alkylene, alkenylene, or alkynylene, each of which is independently substituted or unsubstituted, or C═O, C═S, C═CR¹⁴R¹⁵, C═NR¹⁴, or a bond;
each R¹, R^{1g}, and R^{1h} is independently alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

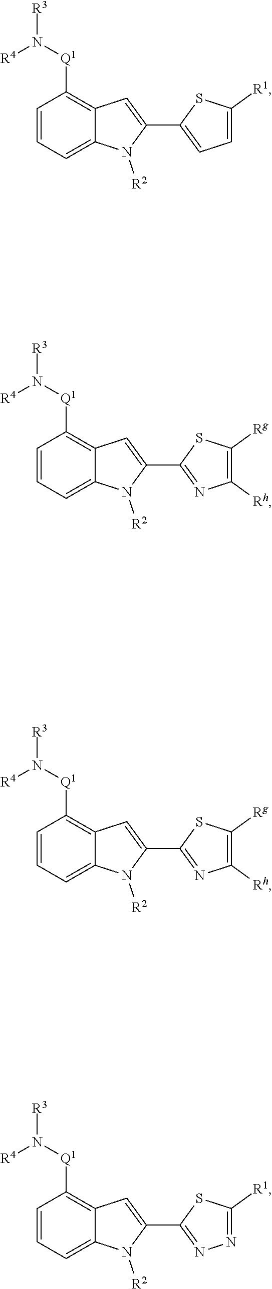

or a pharmaceutically-acceptable salt thereof, wherein the variables are as defined above.

In some embodiments, the compound is of the formula:

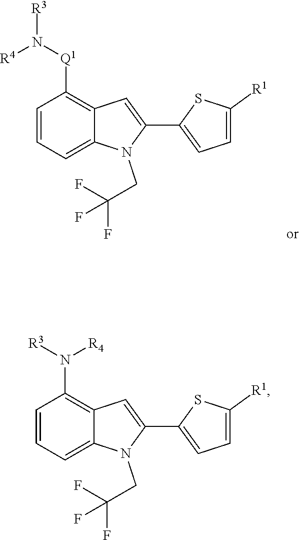

or a pharmaceutically-acceptable salt thereof, wherein the variables are as defined above.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

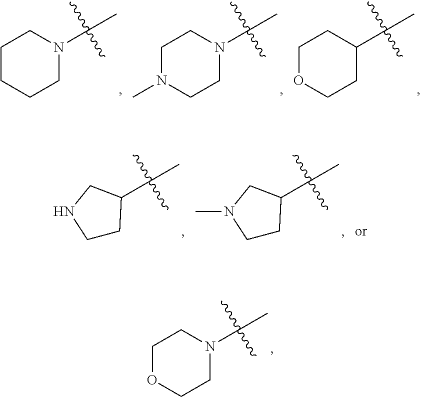

wherein the ring is substituted or unsubstituted. In some embodiments, R³ is H, and R⁴ is a ring that is

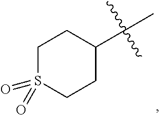 [SMILES: *C1CCS(=O)(=O)CC1]

wherein the ring is substituted or unsubstituted. In some embodiments, R³ is H, and R⁴ is a ring that is

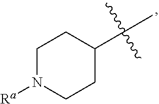

wherein the ring is substituted or unsubstituted. In some embodiments, R^{a} is alkylene. In some embodiments, R^{a} is methyl. In some embodiments, R³ is H, and R⁴ is a ring that is

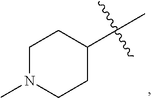 [SMILES: *C1CCN(C)CC1]

wherein the ring is substituted or unsubstituted. In some embodiments, R³ is H, and R⁴ is a ring that is

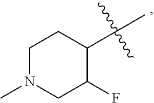 [SMILES: *C1CCN(C)CC1F]

wherein the ring is substituted or unsubstituted.

In some embodiments, R¹ is alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group. In some embodiments, R¹⁶ is hydrogen, and R¹⁷ is carboxyl substituted with alkyl or aryl. In some embodiments, R¹⁶ is hydrogen, and R¹⁷ is carboxyl substituted with cycloalkyl or phenyl. In some embodiments, R¹⁶ and R¹⁷ are hydrogen.

In some embodiments, the compound is of the formula:

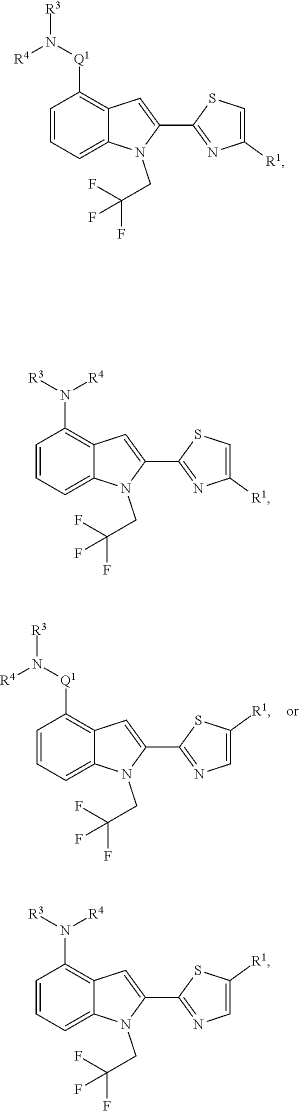

or a pharmaceutically-acceptable salt thereof, wherein the variables are as defined above.

In some embodiments, R¹ is alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group. In some embodiments, R¹⁶ is hydrogen, and R¹⁷ is carboxyl substituted with alkyl or aryl. In some embodiments, R¹⁶ is hydrogen, and R¹⁷ is carboxyl substituted with cycloalkyl or phenyl. In some embodiments, R¹⁶ and R¹⁷ are hydrogen.

In some embodiments, the compounds is of the formula:

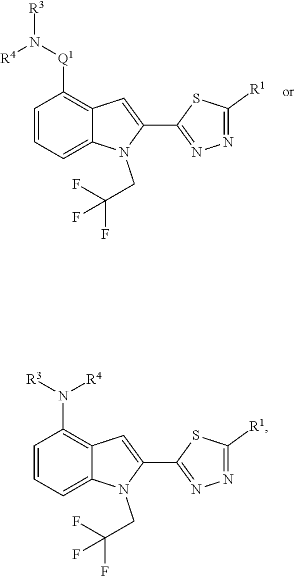

or a pharmaceutically-acceptable salt thereof, wherein the variables are as defined above.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; or —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

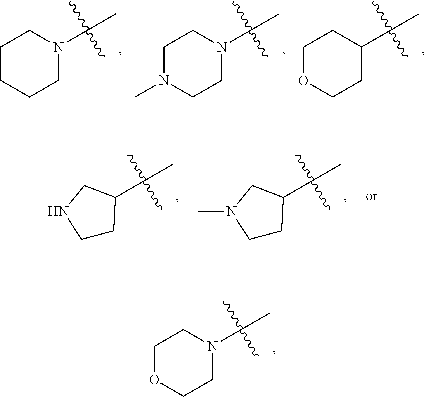

wherein the ring is substituted or unsubstituted. In some embodiments, R³ is H, and R⁴ is a ring that is

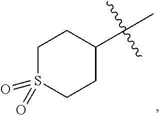 [SMILES: *C1CCS(=O)(=O)CC1]

wherein the ring is substituted or unsubstituted. In some embodiments, R³ is H, and R⁴ is a ring that is

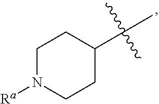

wherein the ring is substituted or unsubstituted. In some embodiments, R^{a} is alkylene. In some embodiments, R^{a} is methyl. In some embodiments, R³ is H, and R⁴ is a ring that is

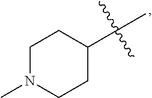 [SMILES: *C1CCN(C)CC1]

wherein the ring is substituted or unsubstituted. In some embodiments, R³ is H, and R⁴ is a ring that is

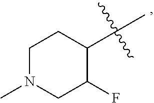 [SMILES: *C1CCN(C)CC1F]

wherein the ring is substituted or unsubstituted.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, halogen, or hydrogen. In some embodiments R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is aryl, heteroaryl, carboxyl, or hydrogen. In some embodiments, R¹⁶ is hydrogen, and R¹⁷ is carboxyl substituted with aryl, heteroaryl, cycloalkyl, or alkyl. In some embodiments, R¹⁶ and R¹⁷ are hydrogen.

In some embodiments, the compound is of the formula:

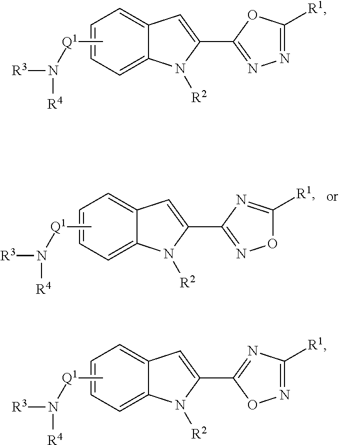

wherein:
Q¹ is alkylene, alkenylene, or alkynylene, each of which is independently substituted or unsubstituted, or C═O, C═S, C═CR¹⁴R¹⁵, C═NR¹⁴, or a bond;
R¹ is alkyl, alkenyl, alkynyl, alkoxy, aryl, heteroaryl, heterocyclyl, or halo, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R², R¹⁴, R¹⁵, R¹⁶, R¹, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

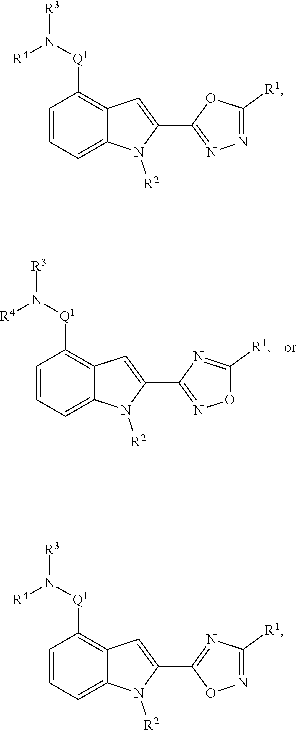

or a pharmaceutically-acceptable salt thereof, wherein the variables are as defined above.

In some embodiments, the compound is of the formula:

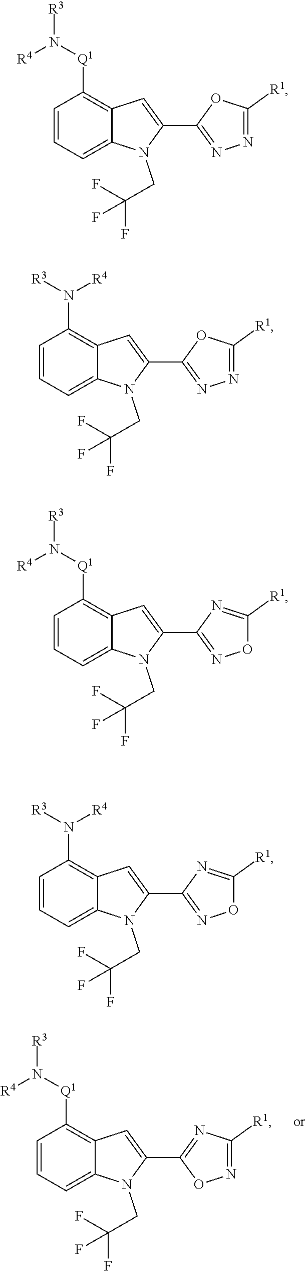

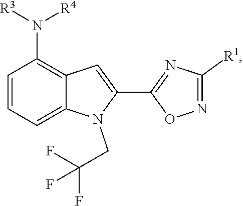

or a pharmaceutically-acceptable salt thereof, wherein the variables are as defined above.

In some embodiments, the compound is of the formula:

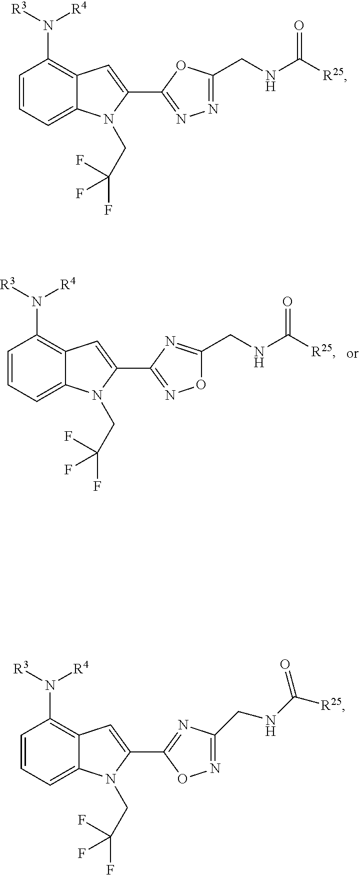

wherein:
Q¹ is alkylene, alkenylene, or alkynylene, each of which is independently substituted or unsubstituted, or C═O, C═S, C═CR¹⁴R¹⁵, C═NR¹⁴, or a bond;
each R^{1c} and R^{1d} is independently alkyl, alkenyl, alkynyl, alkoxy, aryl, or heteroaryl, heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, halogen, or hydrogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted;
each R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
R²⁵ is alkyl, alkenyl, alkynyl, aryl, heteroaryl, or heterocyclyl, each of which is independently substituted or unsubstituted, or hydrogen;
   or a pharmaceutically-acceptable salt thereof.

In some embodiments, R²⁵ is heterocyclyl, cycloalkyl, aryl, each of which is substituted or unsubstituted. In some embodiments, R²⁵ is phenyl or cyclopropyl, each of which is substituted or unsubstituted. In some embodiments, R²⁵ is substituted cyclopropyl. In some embodiments, R²⁵ is heteroaryl or heterocyclyl, each of which is substituted or unsubstituted. In some embodiments, R²⁵ is thiophenyl, indolenyl, or pyrrolyl, each of which is substituted or unsubstituted.

Non-limiting examples of compounds of the disclosure include compounds of any of the following formulae:

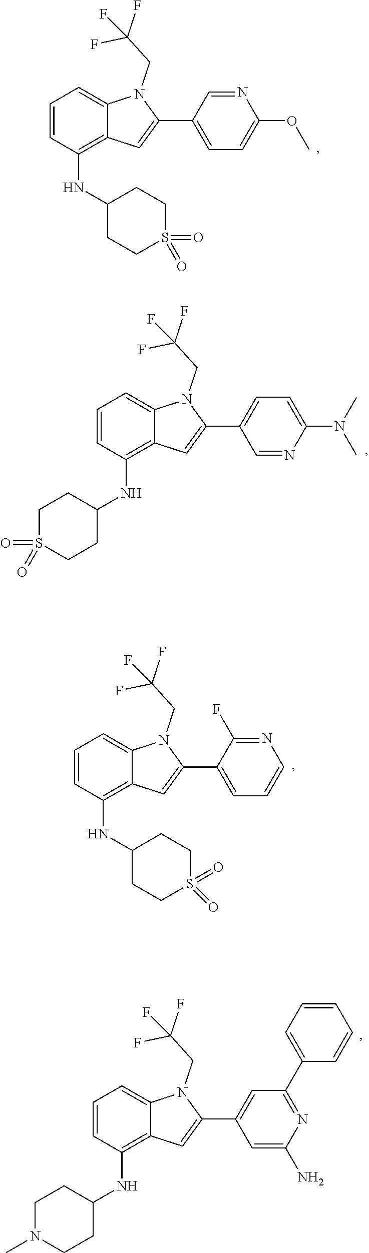

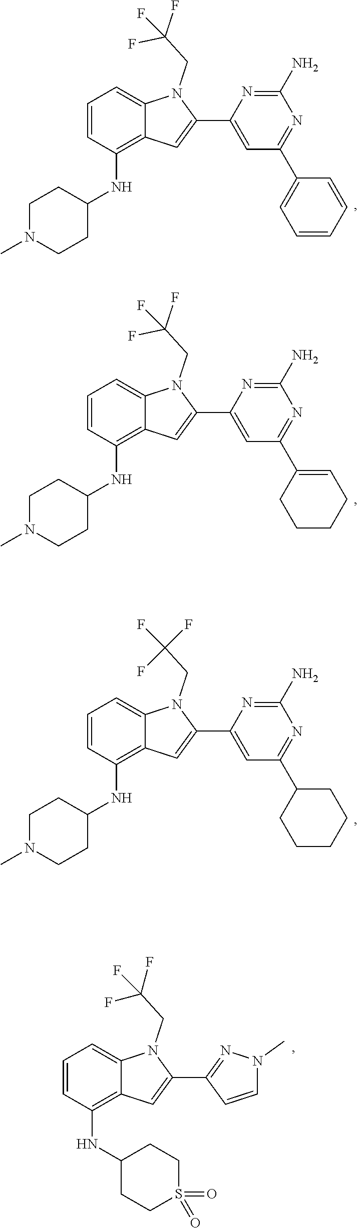 [SMILES: CN1CCC(Nc2cccc3c2cc(-c2cc(-c4ccccc4)nc(N)n2)n3CC(F)(F)F)CC1 ; CN1CCC(Nc2cccc3c2cc(-c2cc(C4=CCCCC4)nc(N)n2)n3CC(F)(F)F)CC1 ; CN1CCC(Nc2cccc3c2cc(-c2cc(C4CCCCC4)nc(N)n2)n3CC(F)(F)F)CC1 ; Cn1ccc(-c2cc3c(NC4CCS(=O)(=O)CC4)cccc3n2CC(F)(F)F)n1]

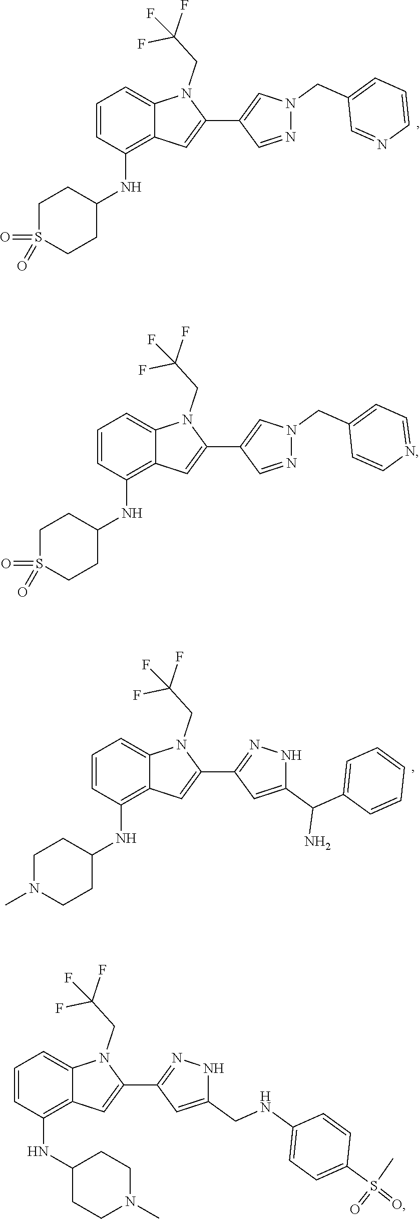 [SMILES: O=S1(=O)CCC(Nc2cccc3c2cc(-c2cnn(Cc4cccnc4)c2)n3CC(F)(F)F)CC1 ; O=S1(=O)CCC(Nc2cccc3c2cc(-c2cnn(Cc4ccncc4)c2)n3CC(F)(F)F)CC1 ; CN1CCC(Nc2cccc3c2cc(-c2cc(C(N)c4ccccc4)[nH]n2)n3CC(F)(F)F)CC1 ; CN1CCC(Nc2cccc3c2cc(-c2cc(CNc4ccc(S(C)(=O)=O)cc4)[nH]n2)n3CC(F)(F)F)CC1]

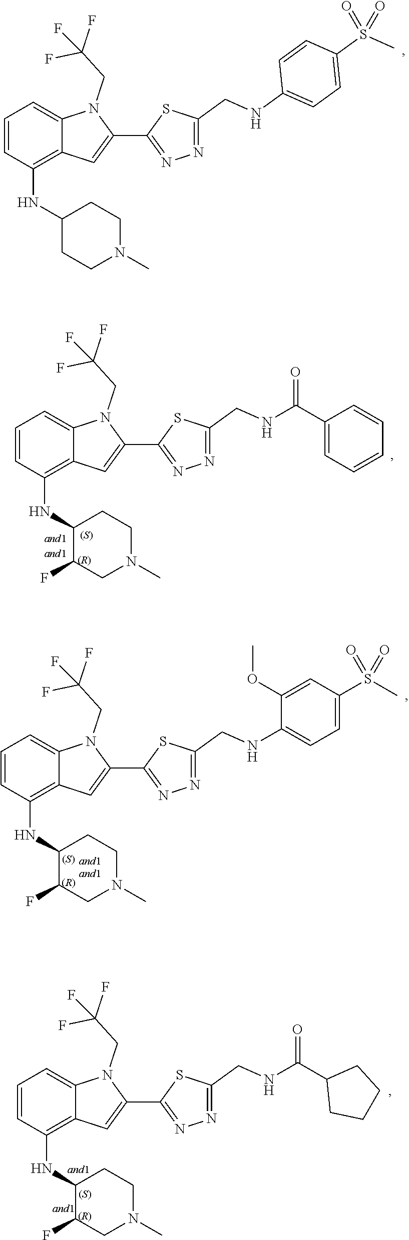

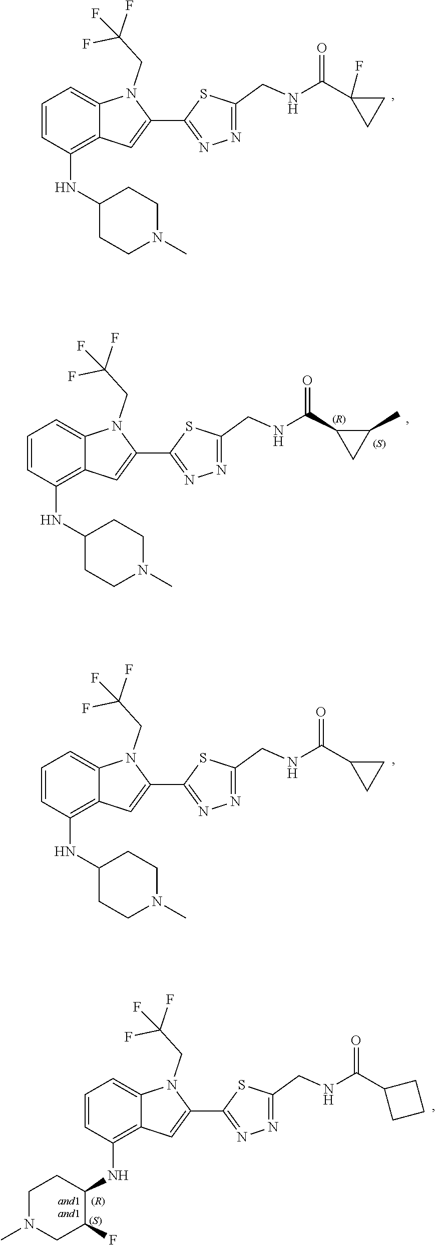

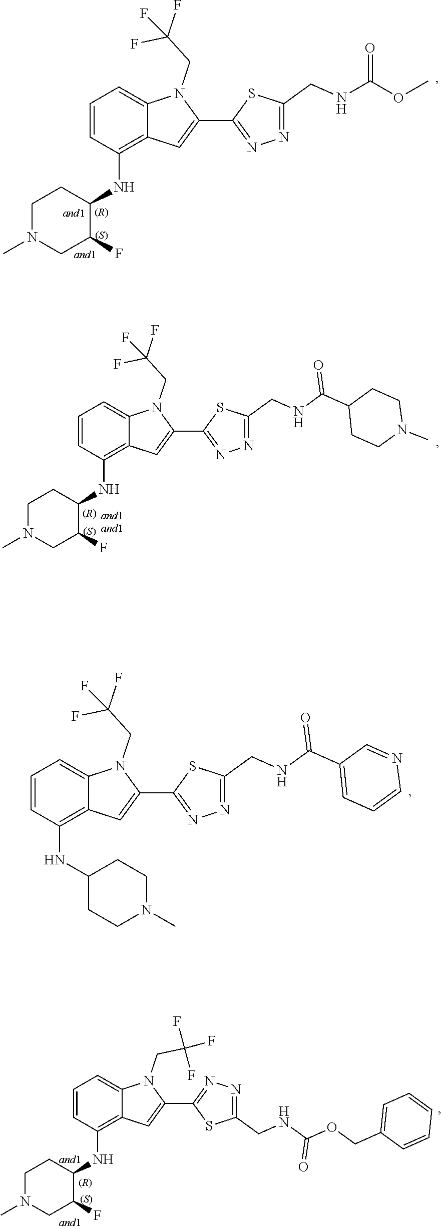

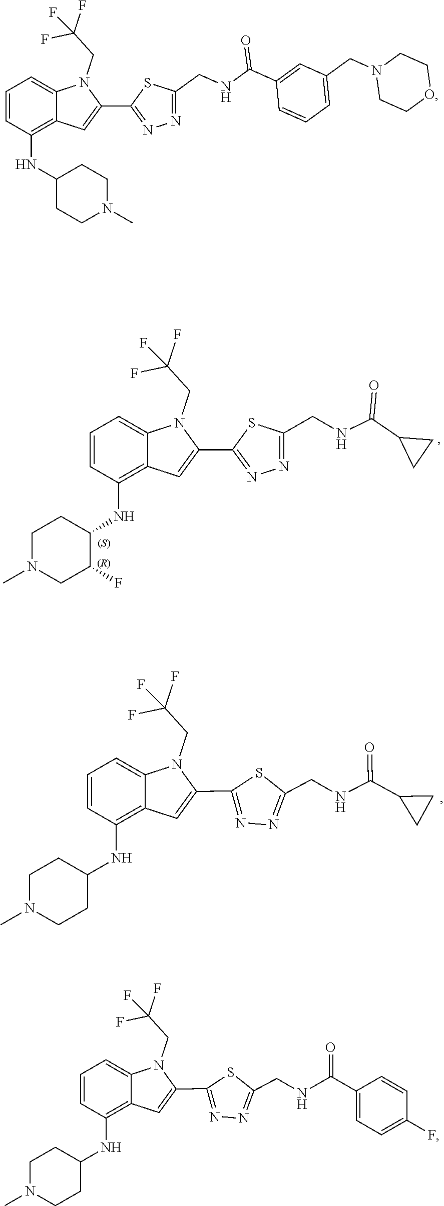

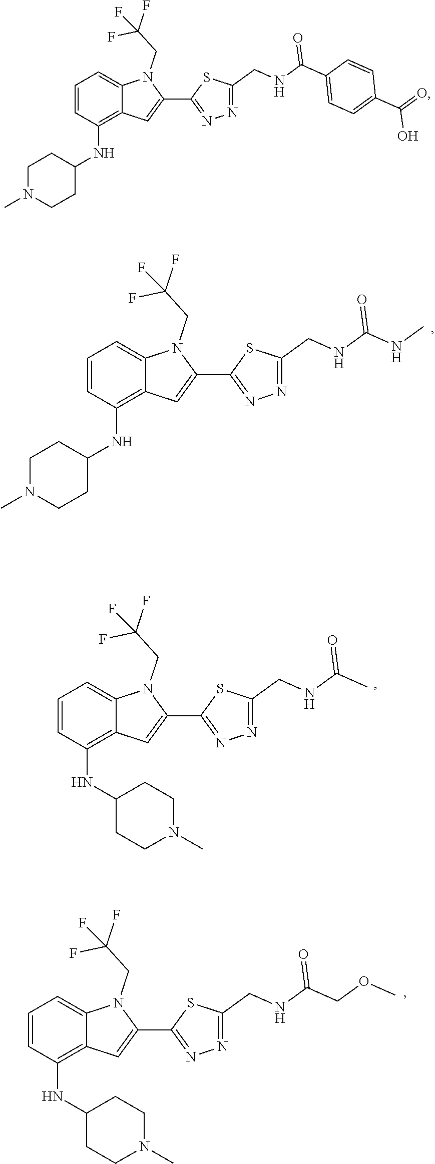

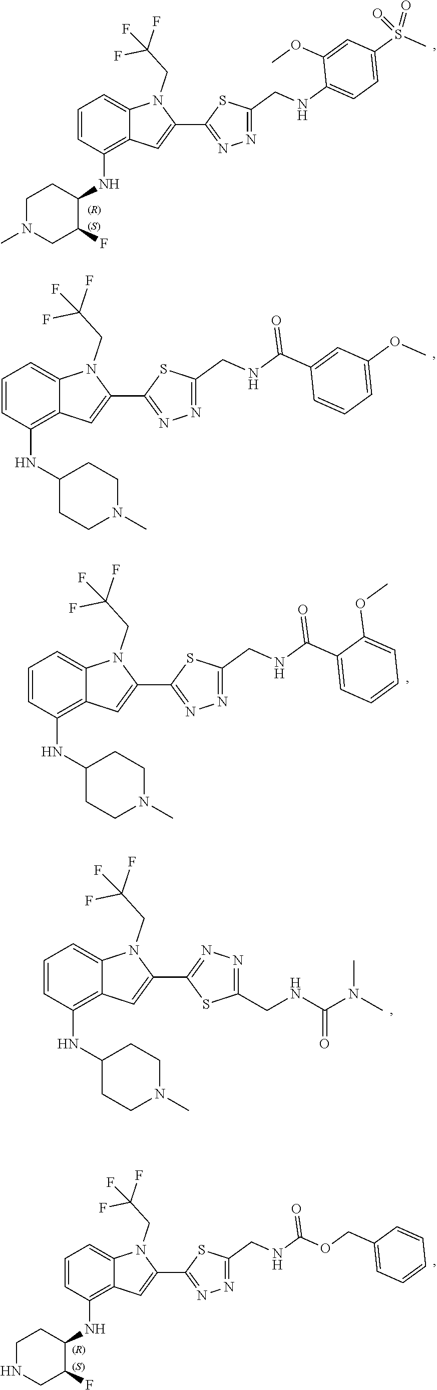

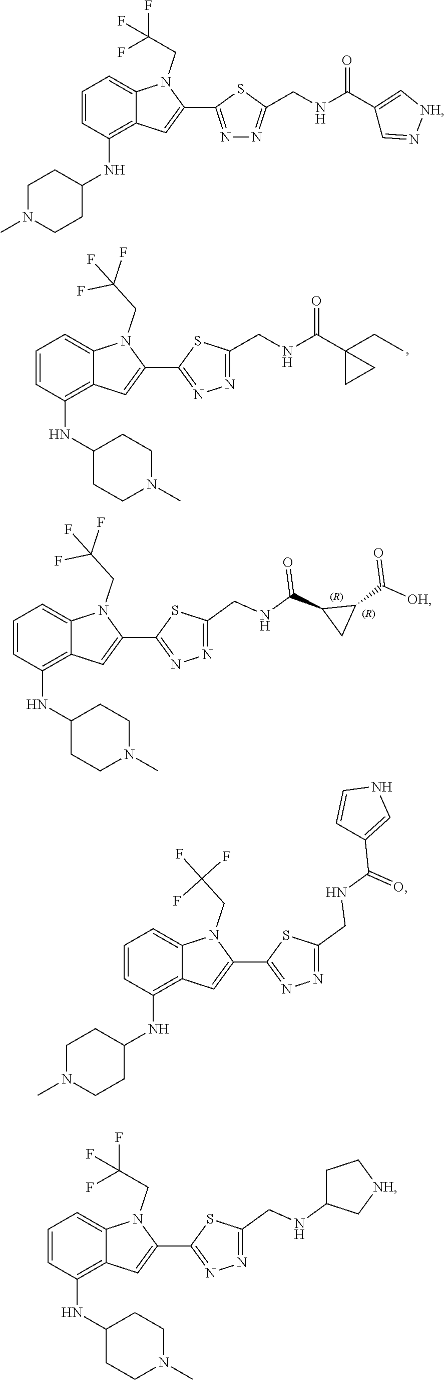

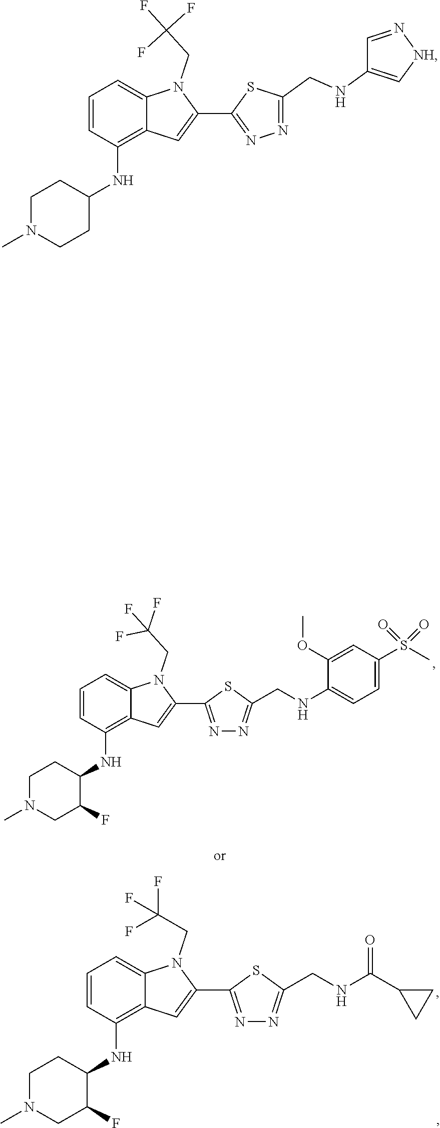

or a pharmaceutically-acceptable salt thereof.

Non-limiting examples of compounds of the disclosure include compounds of any of the following formulae:

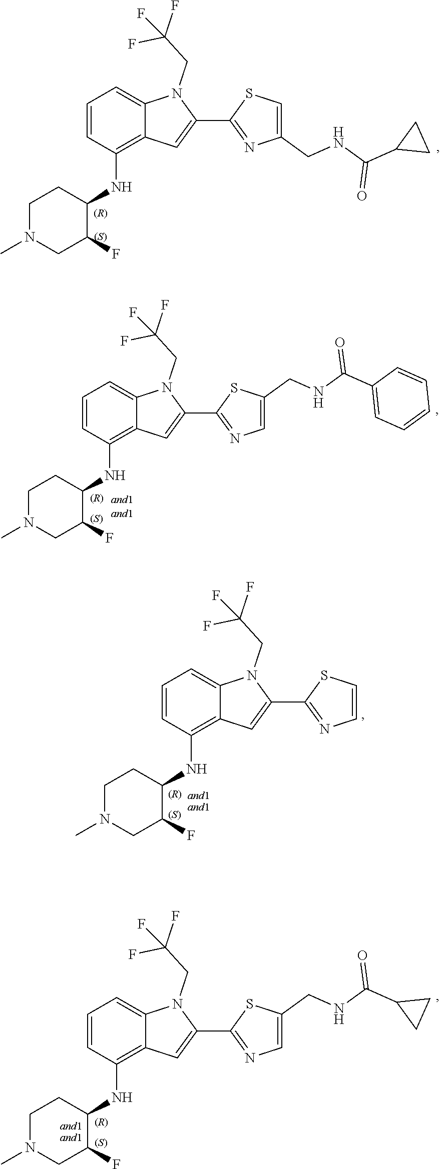

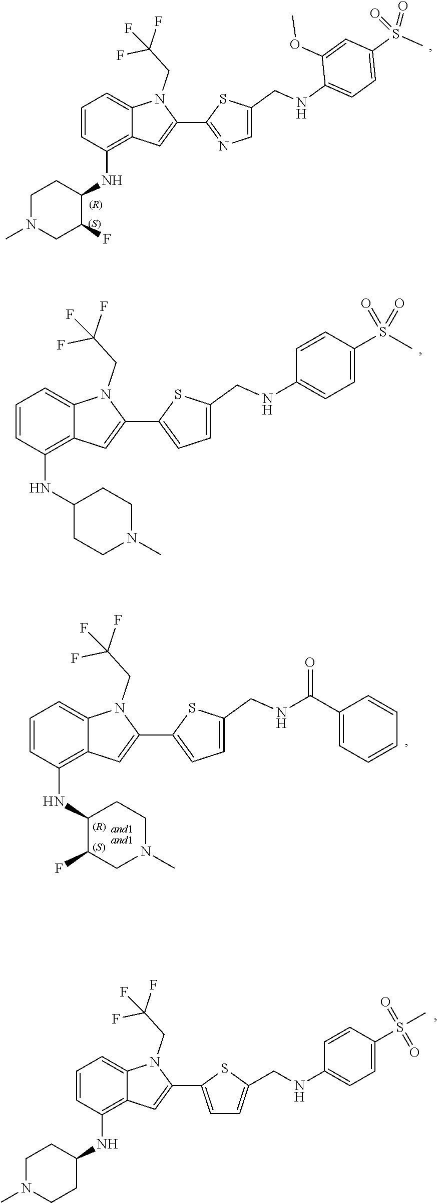

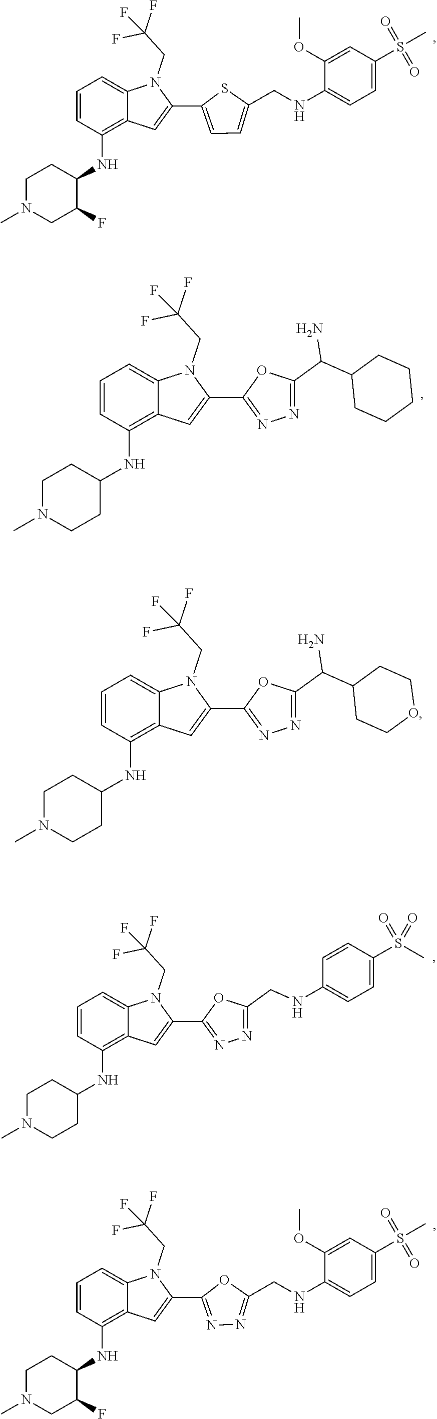

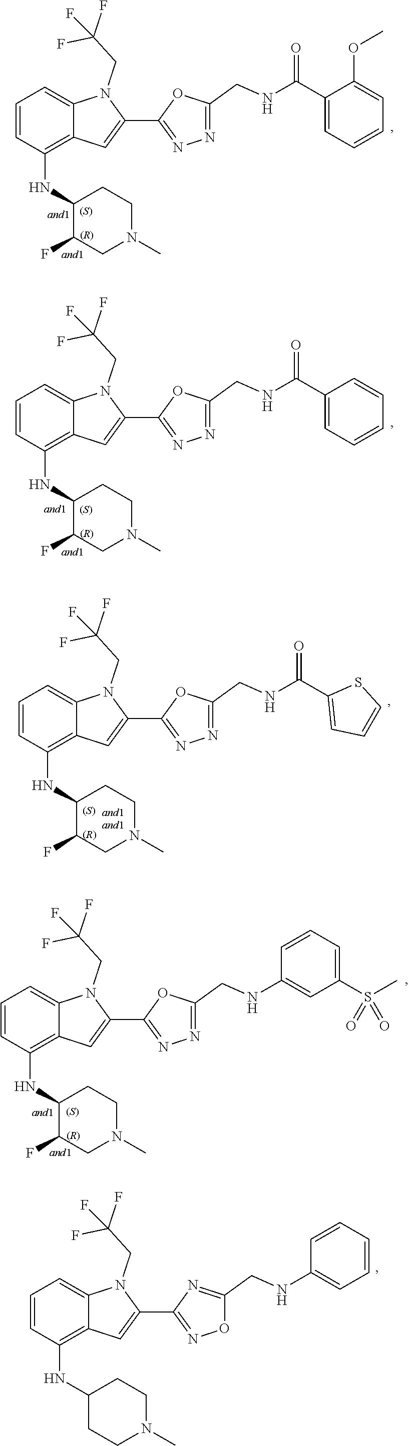

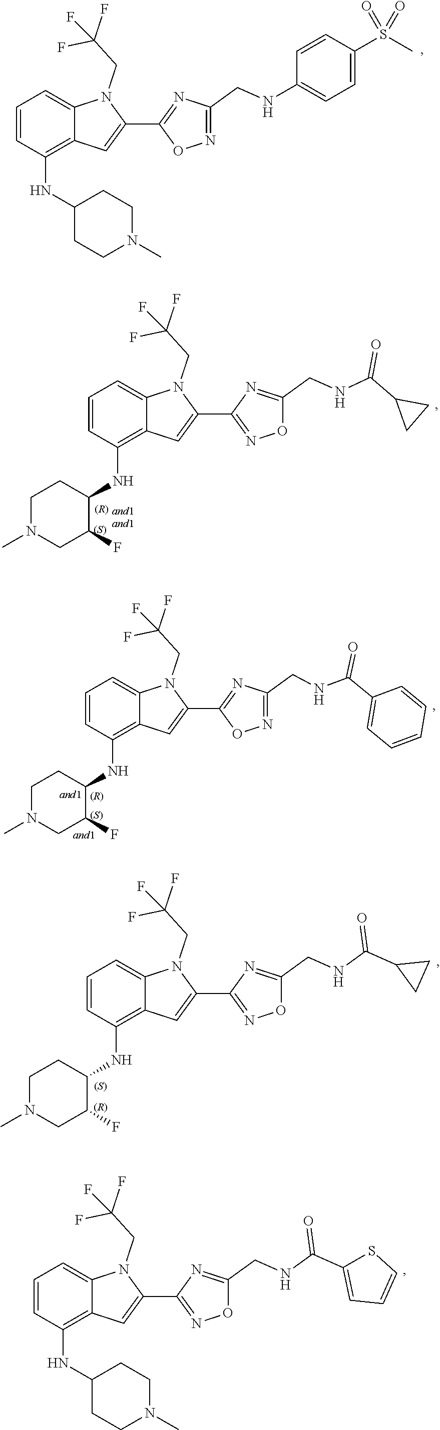

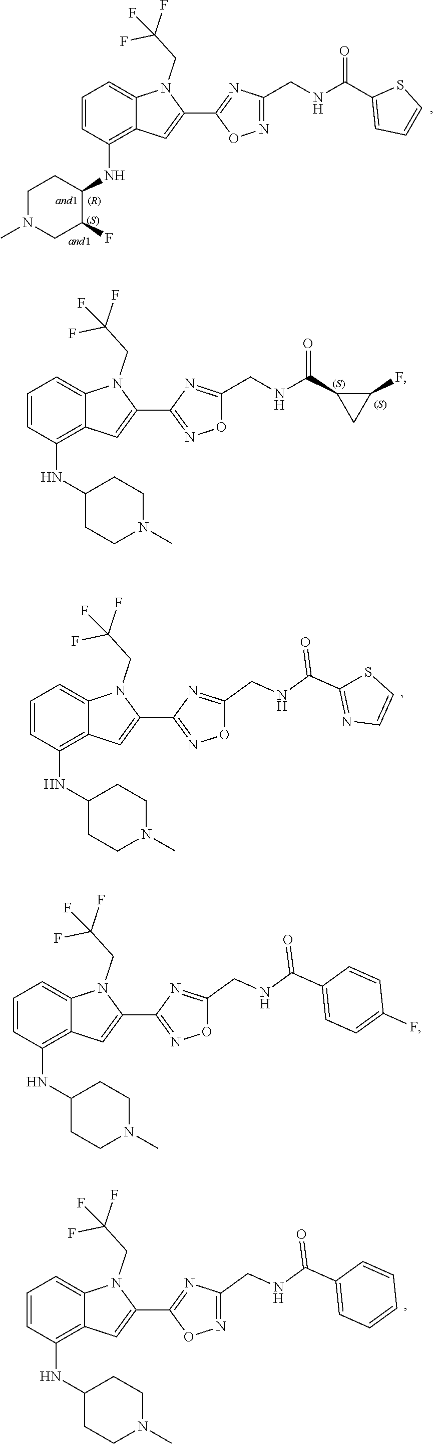

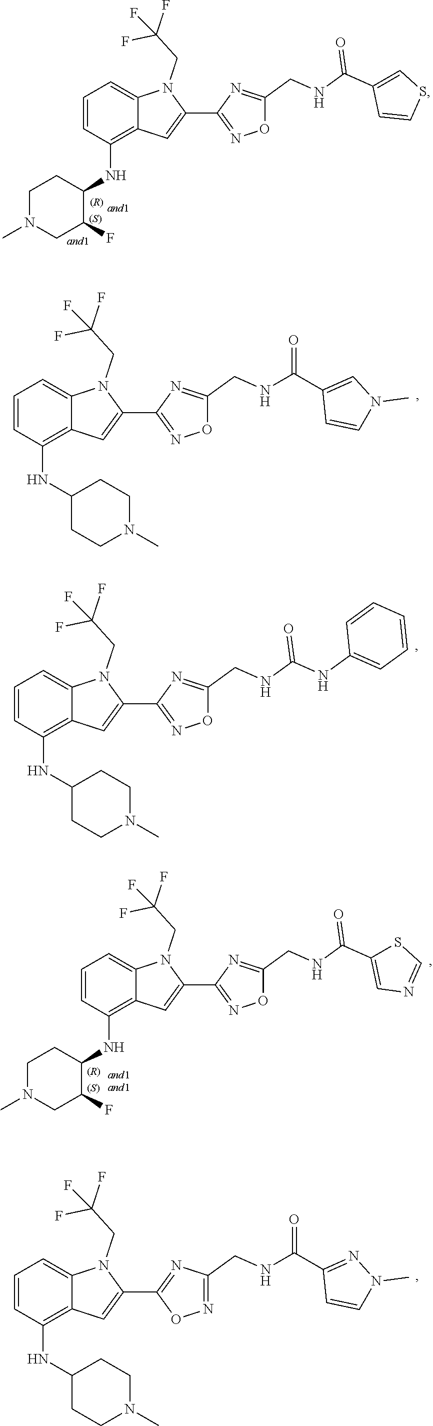

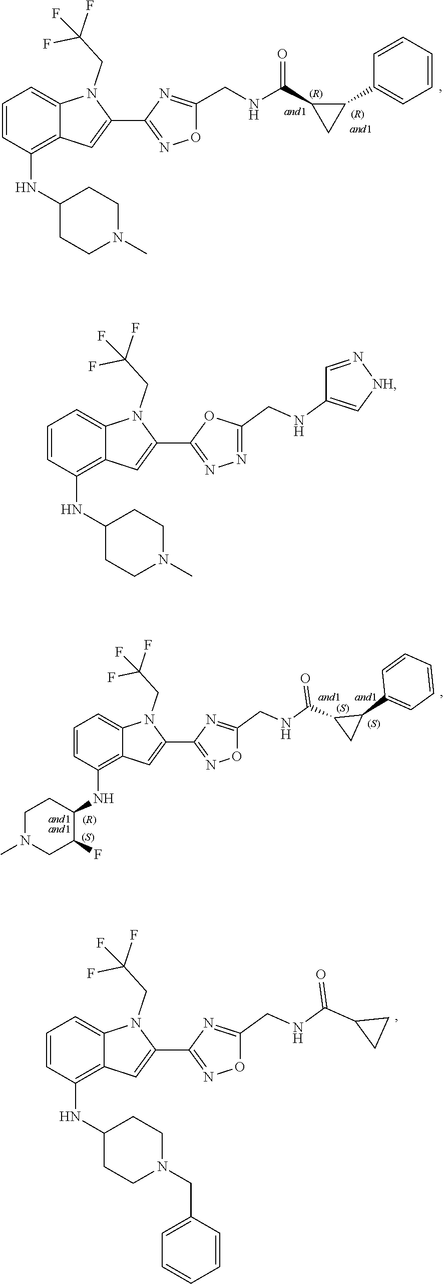

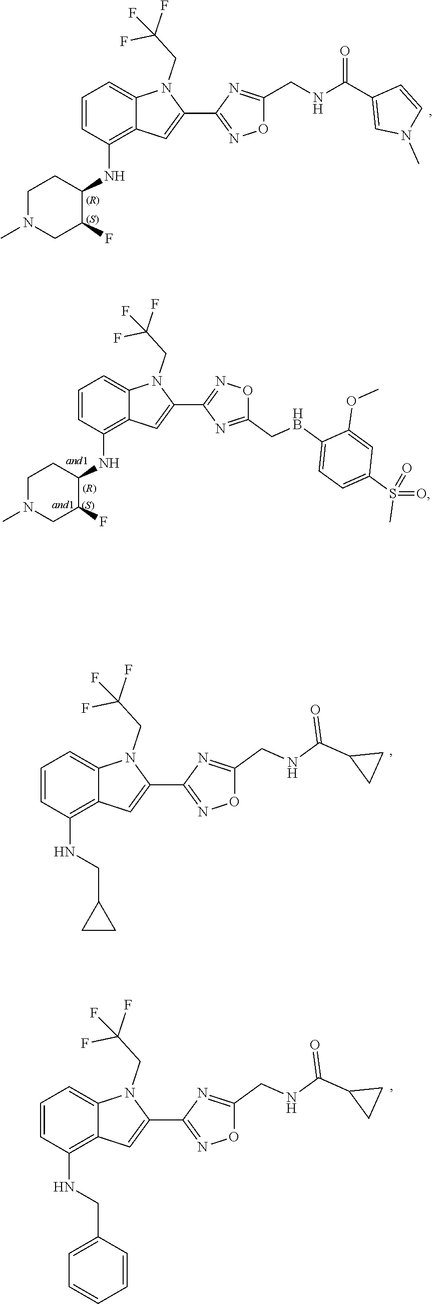

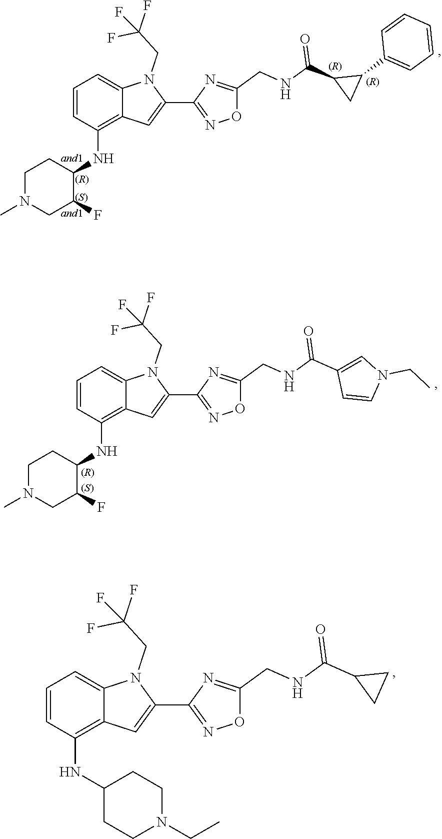

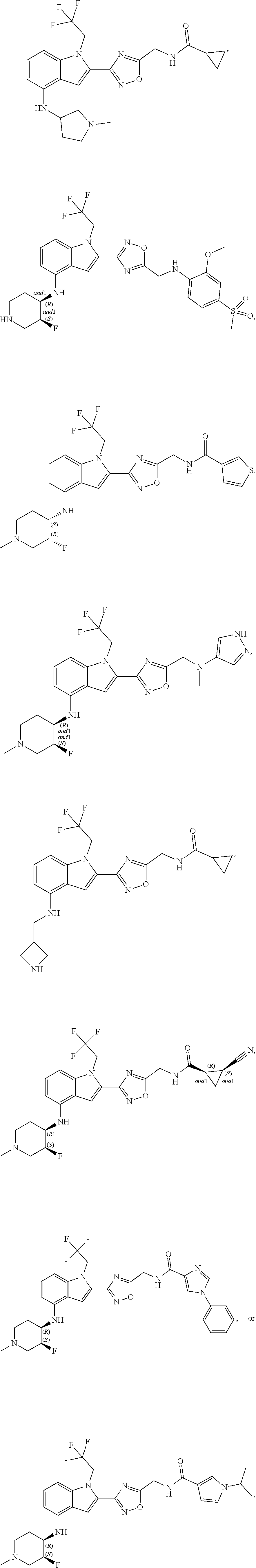

or a pharmaceutically-acceptable salt thereof.

Suitable p53-activating compounds of the present disclosure also can include, for example, p53-activating compounds provided in WO2023025324A1, WO2023165523A1, WO2023016434A1, WO2022213975A1, CN115960094A, CN116217562A, CN115677722A, and CN115504995A, each of which is incorporated by reference in its entirety. Suitable p53-activating compounds of the present disclosure also can include, for example, p53-activating compounds provided in WO2023147419, WO2024041503, and CN2024117586229A, each of which is incorporated by reference in its entirety.

In some embodiments, the compound is of the formula:

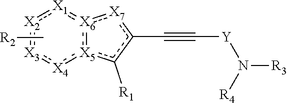

or a pharmaceutically acceptable salt thereof.
wherein:
each [symbol] is independently a single bond or a double bond;
X¹ is CR⁵, NR⁶, O, S, C═O, C═S or a carbon atom connected to R²;
X² is CR⁷, NR⁸, O, S, C═O, C═S or a carbon atom connected to R²;
X³ is CR⁹, NR¹⁰, O, S, C═O, C═S or a carbon atom connected to R²;
X⁴ is CR¹¹, NR¹², O, S, C═O, C═S or a carbon atom connected to R²;
X⁵ and X⁶ are each independently selected from CR¹³ and N;
X⁷ is CR¹⁴ or NR¹⁴;
wherein at least one of X¹, X², X³ and X⁴ is a carbon atom connected to R²;
at least one of X⁵ and X⁶ is N;
R₁ is H, alkyl, cycloalkyl, haloalkyl, halogen, hydroxyl, alkoxyl, —SR¹⁵, —S(O)R¹⁵, —S(O)₂R¹⁵, nitro, nitroso, cyano, amino, carboxyl, —C(O)OR¹⁵, —NR¹⁶R¹⁷, aryl, heteroaryl or heterocyclyl; wherein said alkyl, cycloalkyl, alkoxyl, aryl, heteroaryl or heterocyclyl is optionally substituted with one or more R¹⁸;
R² is alkyl, cycloalkyl, —NR¹⁹R²⁰, —C₁₋₆alky-NR¹⁹R²⁰, haloalkyl, halogen, hydroxyl, alkoxyl, —C(O)NR¹⁹R²⁰, aryl, heteroaryl or heterocyclyl; wherein said alkyl, alkoxyl, cycloalkyl, aryl, heteroaryl and heterocyclyl is optionally substituted by one or more R²¹;
R³ is H, hydroxyl, halogen, nitro, cyano, carboxyl, amino, alkyl, alkoxyl, haloalkyl or cycloalkyl;
R⁴ is aryl, heteroaryl, each of which is optionally substituted by one or more R²², or

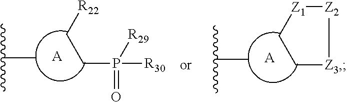

A is aryl or heteroaryl, each of which is optionally substituted with one or more R²²;
Z¹, Z², and Z³ are each independently selected from CR′R″, O, S, S(O)₂, and NR′;
each R′ and R″ is independently H, hydroxyl, halogen, nitro, cyano, carboxyl, amino, alkyl, alkoxyl, haloalkyl, or cycloalkyl;
each R₅, R⁶, R⁸, R⁹, R¹⁰, R¹¹, R¹², and R¹³ is independently H, alkyl, halogen, haloalkyl, cycloalkyl, hydroxyl, nitro, amino or alkoxyl;
each R¹⁴, R¹⁶, R¹⁷, R¹⁹, R²⁰ is independently H, alkyl, cycloalkyl, haloalkyl, halogen, hydroxyl, alkoxyl —SR¹⁵, —S(O)R¹⁵, —S(O)₂R¹⁵, nitro, nitroso, cyano, amino, carboxyl, —C(O)OR¹⁵, —NR¹⁶R¹⁷, aryl, heteroaryl and heterocyclyl, wherein each alkyl, cycloalkyl, aryl, heteroaryl and heterocyclyl is optionally substituted by one or more R²³;
or R¹⁶ and R¹⁷, together with N atom to which R¹⁶ and R¹⁷ are bound form a 3- to 6-membered heterocyclyl, said heterocyclyl is optionally substituted by one or more R²⁴, and each R¹⁴, R¹⁹, and R²⁰ is independently H, alkyl, cycloalkyl, haloalkyl, halogen, hydroxyl alkoxyl, —SR¹⁵, —S(O)R¹⁵, —S(O)₂R¹⁵, nitro, nitroso, cyano, amino, carboxyl, —C(O)OR¹⁵, —NR¹⁶R¹⁷, aryl, heteroaryl or heterocyclyl, wherein each alkyl, cycloalkyl, aryl, heteroaryl and heterocyclyl is optionally substituted by one or more R23
or R¹⁹ and R²⁰, together with N atom to which R¹⁹ and R²⁰ are bound form a 3- to 6-membered heterocyclyl, said heterocyclyl is optionally substituted by one or more R²⁵, and each R¹⁴, R¹⁶ and R¹⁷, is independently H, alkyl, cycloalkyl, haloalkyl, halogen, hydroxyl, alkoxyl, —SR¹⁵, —S(O)R¹⁵, —S(O)₂R¹⁵, nitro, nitroso, cyano, amino, carboxyl, —C(O)O¹⁵, —NR¹⁶R⁷¹, aryl, heteroaryl or heterocyclyl, wherein each alkyl, cycloalkyl, aryl, heteroaryl and heterocyclyl is optionally substituted by one or more R²³; Y is (C(R¹⁵)₂)ₘ;
m is, 0, 1, 2, or 3;
each R¹⁵ is independently H, hydroxyl, alkyl, cycloalkyl, or halogen;
R¹⁸ is halogen, cycloalkyl, alkyl, nitro, cyano, alkoxyl or hydroxyl;
R²³ is —NR²⁶R²⁷, alkyl, cycloalkyl, haloalkyl, halogen, hydroxyl, nitro, carboxyl, —C(O)C₁₋₃alkylNR²⁶R²⁷, —C(O)NR²⁶R²⁷, heterocyclyl, aryl, or heteroaryl, wherein alkyl, cycloalkyl, aryl, heteroaryl and heterocyclyl are each optionally substituted by one or more substituents selected from the group consisting of alkyl, alkoxyl, hydroxyl, amino and halogen;
each R²¹, R²², R²⁴, R²⁵ is independently selected from H, alkyl, alkenyl, alknyl, cycloalkyl, alkoxyl, hydroxyl, amino, alkylamino-, nitro, carboxyl, cyano, halogen, —C(O)OR²⁸, —C(O)NR²⁹R³⁰, —C₁₋₃alkyl-C(O)NR²⁹R³⁰, —C(O)C₁₋₃alkyl-NR²⁹R³⁰, —S(O)₂R²⁸, —S(O)R²⁸, —S(O)₂NR²⁹R³⁰, —P(O)R²⁹R³⁰, aryl, heteroaryl and heterocyclyl, wherein aryl, heteroaryl and heterocyclyl are each optionally substituted by one or more substituents selected from the group consisting of halogen, hydroxyl, amino, alkyl and alkoxyl;
each R²⁶, R²⁷, R²⁸, R²⁹, R³⁰ is independently selected from H, hydroxyl, alkyl, hydroxylalkyl, alkoxyl, amino, aminoalkyl, cycloalkyl and halogen;
or R²⁹ and R³⁰, along with the N or P atom to which R²⁹ and R³⁰ are attached form a 3- to 6-membered ring which is optionally substituted by one or more substituents independently selected from —C₁₋₆ alkyl, and each R²⁶, R²⁷, and R²⁸ is independently selected from H, hydroxyl, alkyl, hydroxylalkyl, alkoxyl, amino, aminoalkyl, cycloalkyl and halogen.

In some embodiments, the compound is of the following:

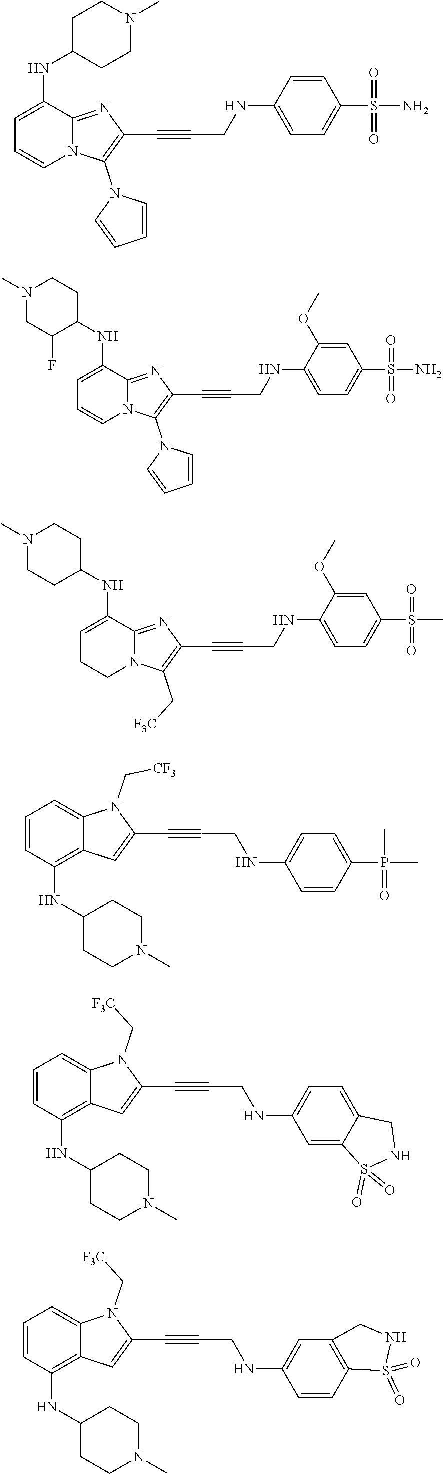

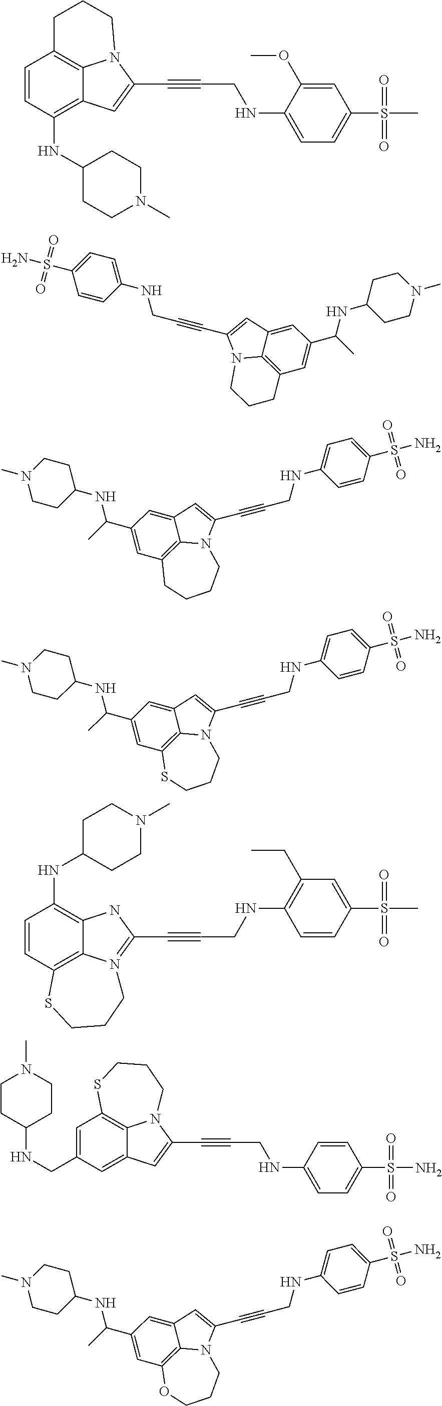

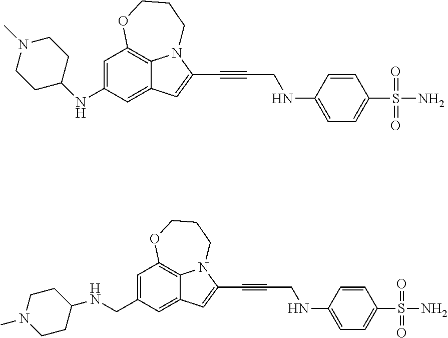 [SMILES: CN1CCC(Nc2cc3c4c(c2)cc(C#CCNc2ccc(S(N)(=O)=O)cc2)n4CCCO3)CC1 ; CN1CCC(NCc2cc3c4c(c2)cc(C#CCNc2ccc(S(N)(=O)=O)cc2)n4CCCO3)CC1]

In some embodiments, the compound is of the formula:

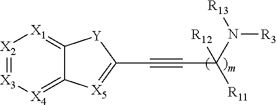

Y is selected from O, S, NR′, —S═O, —S(═O)(=NR′)— or S(═O)₂;
one of X¹, X², X³ and X⁴ is selected from CR², and the others of X¹, X², X³ and X⁴ are each independently selected from N or CR⁴;
X⁵ is selected from N or CR¹;
R¹ is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC O) R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl and 5-12 membered heteroaryl;
R₂ is —NR₅₁R₅₂, —OR₅₃ or —SR₅₄;
R₃ is selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) R^{3a};
each R^{3a} is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(=NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more substituents R^{3b},
wherein each R^{3b} is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(=NR′)R′, —NR′S(O)₂R′, —NR′S(O)₂N(R′)₂, and —PO(R′)₂;
R⁴ at each occurrence is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O) N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)R′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(=NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl;
R⁵¹, R⁵², R⁵³ and R⁵⁴ are each independently selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, =NR′, —C₁₋₆alkyl-CH(R′)₂, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O) N(R′)₂, —NR′S(O)R′, —NR′S(O)N(R′)₂, —S(O)₂R′, —S(O)₂N(R′)₂, —S(═O)(=NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl;
R¹¹ and R¹² are independently selected from hydrogen, deuterium, —OH, halogen, —CN, oxo, —C₁₋₆alkyl, —C₁₋₆ haloalkyl, —C₁₋₆alkoxy, —NH₂, —NHC₁₋₆alkyl, —N(C₁₋₆alkyl)₂, and 3-6 membered cycloalkyl; wherein said —C₁₋₆alkyl, —C₁₋₆alkoxy and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH—C₁₋₆alkyl, —N(C₁₋₄ alkyl)₂, and 3-6 membered cycloalkyl; R¹³ is selected from hydrogen, deuterium, —C₁₋₆alkyl and 3-6 membered cycloalkyl; wherein said —C₁₋₆alkyl and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH₂, —NHC₁₋₆ alkyl, and —N(C₁₋₄alkyl)₂;
each R′ at each occurrence is independently selected from hydrogen, deuterium, halogen, —OH, —CN, oxo, —NH₂, —NHC₁₋₆alkyl, —N(C₁₋₆alkyl)₂, —C₁₋₆alkyl, —C₁₋₆alkylOC₁₋₆alkyl, —C₁₋₆alkyl-NHC₁₋₆alkyl, —C₁₋₆alkyl-N(C₁₋₆alkyl)₂, —C₁₋₆haloalkyl, —OC₁₋₆alkyl, —C₃₋₁₄cycloalkyl, —C₃₋₁₄heterocycloalkyl, —C₂₋₆alkenyl, —C₂₋₆alkynyl, 6-12 membered aryl, and 5-12 membered heteroaryl; wherein said —C₁₋₆alkyl, —OC₁₋₆alkyl, —C₃₋₁₄cycloalkyl, —C₃₋₁₄heterocycloalkyl, —C₂₋₆alkenyl, —C₂₋₆alkynyl, 6-12 membered aryl, and 5-12 membered heteroaryl are each independently optionally substituted with 1, 2, 3, 4, 5 or 6 substituents selected from deuterium, halogen, —CN, —C₁₋₃alkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(=NH)C₁₋₃alkyl, —S(═O)(=NC₁₋₃alkyl)C₁₋₃alkyl, and 3-6 membered cycloalkyl; said heterocycloalkyl, heterocycloalkenyl, and heteroaryl each independently contains 1, 2 or 3 heteroatoms selected from N, O, P and S;
m is selected from 1, 2, 3, 4, 5 and 6.

In some embodiments, the compound is of the following:
4-((3-(3-ethyl-7-((1-methylpiperidin-4-yl)amino)benzo[b]thiophen-2-yl) prop-2-yn-1-yl)amino)benzenesulfonamide
(Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (racemic)
N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine
4-((3-(7-((1-methylpiperidin-4-yl)amino)-3-(2,2,2-trifluoroethyl)benzo[b]thiophen-2-yl) prop-2-yn-1-yl)amino)benzenesulfonamide
4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)benzenesulfonamide (racemic)
4-((3-(7-(((3R,4R)-3-fluoro-1-methylpiperidin-4-yl)amino)-3-(2,2,2-trifluoroethyl)benzo[b]thiophen-2-yl) prop-2-yn-1-yl)amino)benzenesulfonamide (racemic)
1-((Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino) piperidin-1-yl)-3-methoxypropan-2-ol (racemic)
N-(2-(3-((5-fluoro-2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine
3-methoxy-N-methyl-4-((3-(7-((1-methylpiperidin-4-yl)amino)-3-(2,2,2-trifluoroethyl)benzo[b]thiophen-2-yl) prop-2-yn-1-yl)amino)benzamide
dimethyl(4-((3-(7-((1-methylpiperidin-4-yl)amino)-3-(2,2,2-trifluoroethyl)benzo[b]thiophen-2-yl) prop-2-yn-1-yl)amino)phenyl)phosphine oxide
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-(fluoromethoxy)phenyl)dimethylphosphine oxide (racemic)
N-(2-(3-((2-(fluoromethoxy)-4-(methylsulfonyl)phenyl)amino)prop-1-yn-1-yl)-3-(2,2,2-trifluoroethyl)benzo[b]thiophen-7-yl)-1-methylpiperidin-4-amine
N-(2-(3-((2-(2,2-difluoroethoxy)-4-(methylsulfonyl)phenyl)amino)prop-1-yn-1-yl)-3-(2,2,2-trifluoroethyl)benzo[b]thiophen-7-yl)-1-methylpiperidin-4-amine
1-methyl-N-(2-(3-((4-(methylsulfonyl)-2-(2,2,2-trifluoroethoxy)phenyl)amino)prop-1-yn-1-yl)-3-(2,2,2-trifluoroethyl)benzo[b]thiophen-7-yl) piperidin-4-amine
N-[2-[3-[2-(2-methoxyethoxy)-4-methylsulfonyl-anilino]prop-1-ynyl]-3-(2,2,2-trifluoroethyl)benzothiophen-7-yl]-1-methyl-piperidin-4-amine
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic)
N1-(2-(3-((2-methoxy-4-(methylsulfonyl)phenyl)amino)prop-1-yn-1-yl)-3-(2,2,2-trifluoroethyl)benzo[b]thiophen-7-yl)-N4,N4-dimethylcyclohexane-1,4-diamine
Trans-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
Trans-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
cis-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
Trans-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol)
cis-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol)
N-[2-[3-(2-methoxy-4-methylsulfonyl-anilino) prop-1-ynyl]-3-(2,2,2-trifluoroethyl)benzothiophen-7-yl]-1-(3-methoxypropyl)piperidin-4-amine
3-methoxy-N,N-dimethyl-4-((3-(7-((1-methylpiperidin-4-yl)amino)-3-(2,2,2-trifluoroethyl)benzo[b]thiophen-2-yl) prop-2-yn-1-yl)amino)benzenesulfonamide
N-(2-(3-((2-methoxy-4-(morpholinosulfonyl)phenyl)amino)prop-1-yn-1-yl)-3-(2,2,2-trifluoroethyl)benzo[b]thiophen-7-yl)-1-methylpiperidin-4-amine
3-methoxy-4-((3-(7-((1-methylpiperidin-4-yl)amino)-3-(2,2,2-trifluoroethyl)benzo[b]thiophen-2-yl) prop-2-yn-1-yl)amino)benzenesulfonamide
N-(2,3-dihydroxypropyl)-3-methoxy-4-((3-(7-((1-methylpiperidin-4-yl)amino)-3-(2,2,2-trifluoroethyl)benzo[b]thiophen-2-yl) prop-2-yn-1-yl)amino)benzenesulfonamide
N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.1]octan-8-amine
N-(2-(3-((2-methoxy-4-(methylsulfonyl)phenyl)amino)prop-1-yn-1-yl)-3-(2,2,2-trifluoroethyl)benzo[b]thiophen-7-yl)-8-methyl-8-azabicyclo[3.2.1]octan-3-amine
N-(2-(3-((2-methoxy-4-(methylsulfonyl)phenyl)amino)prop-1-yn-1-yl)-3-(2,2,2-trifluoroethyl)benzo[b]thiophen-7-yl)-2-methyl-2-azabicyclo[2.2.1]heptan-5-amine
N-(2-(3-((2-methoxy-4-(methylsulfonyl)phenyl)amino)prop-1-yn-1-yl)-3-(2,2,2-trifluoroethyl)benzo[b]thiophen-7-yl)-2-azabicyclo[2.2.1]heptan-5-amine
N-(3-(7-((1-methylpiperidin-4-yl)amino)-3-(2,2,2-trifluoroethyl)benzo[b]thiophen-2-yl) prop-2-yn-1-yl)-1H-indol-7-amine
N-(3-(7-((1-methylpiperidin-4-yl)amino)-3-(2,2,2-trifluoroethyl)benzo[b]thiophen-2-yl) prop-2-yn-1-yl)-3-(piperidin-4-yl)-1H-indol-7-amine
N-(3-(7-((1-methylpiperidin-4-yl)amino)-3-(2,2,2-trifluoroethyl)benzo[b]thiophen-2-yl) prop-2-yn-1-yl)-4-(methylsulfonyl)-1H-indol-7-amine
N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.0]heptan-6-amine
N-(2-(3-((2-methoxy-4-(methylsulfonyl)phenyl)amino)prop-1-yn-1-yl)-3-(2,2,2-trifluoroethyl)benzo[b]thiophen-7-yl)-7-methyl-7-azaspiro[3.5]nonan-2-amine
3-fluoro-N-(2-(3-((2-methoxy-4-(methylsulfonyl)phenyl)amino) prop-1-yn-1-yl)-3-(2,2,2-trifluoroethyl)benzo[b]thiophen-7-yl)-1-methylpiperidin-4-amine
diethyl(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)phosphineoxide (racemic)
1-(2-fluoroethyl)-N-(2-(3-((2-methoxy-4-(methylsulfonyl)phenyl)amino)prop-1-yn-1-yl)-3-(2,2,2-trifluoroethyl)benzo[b]thiophen-7-yl) piperidin-4-amine
N-(2-(3-((2-methoxy-4-(methylsulfonyl)phenyl)amino)prop-1-yn-1-yl)-3-(2,2,2-trifluoroethyl)benzo[b]thiophen-7-yl)-1-(2,2,2-trifluoroethyl) piperidin-4-amine
N-(2-(3-((2-methoxy-4-(methylsulfonyl)phenyl)amino)prop-1-yn-1-yl)-3-(2,2,2-trifluoroethyl)benzo[b]thiophen-7-yl) piperidin-4-amine
Cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine
trans-N-((1R,4R)-4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxybenzenesulfonamide (racemic)
4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)-3-methoxy-N-methylbenzamide (racemic)
(Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-N, 1-dimethylpiperidin-4-amine (racemic)
(4-((3-(7-((3,3-difluoro-1-methylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
(R)-4-((4-(7-((1-methylpiperidin-4-yl)amino)-3-(2,2,2-trifluoroethyl)benzo[b]thiophen-2-yl) but-3-yn-2-yl)amino)benzenesulfonamide
(S)-4-((4-(7-((1-methylpiperidin-4-yl)amino)-3-(2,2,2-trifluoroethyl)benzo[b]thiophen-2-yl) but-3-yn-2-yl)amino)benzenesulfonamide
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxybenzamide (racemic)
4-((3-(7-((3-fluoro-1-methylpiperidin-4-yl)amino)-3-(2,2,2-trifluoroethyl)benzo[b]thiophen-2-yl) prop-2-yn-1-yl)amino)-3-methoxy-N-(1-methylpiperidin-4-yl)benzamide (racemic)
(1,1-dioxidothiomorpholino) (4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)-3-methoxyphenyl) methanone (racemic)
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl) (4-methylpiperazin-1-yl) methanone (racemic)
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl) (morpholino) methanone (racemic)
N-(4-fluoro-2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine
(2-fluoro-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-5-methoxy phenyl)dimethylphosphineoxide (racemic)
2-(3-((2-methoxy-4-(methylsulfonyl)phenyl)amino)prop-1-yn-1-yl)-N-(1-methylpiperidin-4-yl)-3-(2,2,2-trifluoroethyl) thieno[3,2-b]pyridin-7-amine
(3-methoxy-4-((3-(7-(((tetrahydro-1H-pyrrolizin-7a (5H)-yl)methyl)amino)-3-(2,2,2-trifluoroethyl)benzo[b]thiophen-2-yl) prop-2-yn-1-yl)amino)phenyl)dimethylphosphine oxide
N-(2-(3-((2-methoxy-4-(methylsulfonyl)phenyl)amino)prop-1-yn-1-yl)-3-(2,2,2-trifluoroethyl)benzo[b]thiophen-7-yl)-1-methylazepan-4-amine
2-(3-((2-methoxy-4-(methylsulfonyl)phenyl)amino)prop-1-yn-1-yl)-N-((1-methylpiperidin-4-yl)methyl)-3-(2,2,2-trifluoroethyl)benzo[b]thiophen-7-amine
4-((3-(4-(((Z)-3-fluoro-1-methylpiperidin-4-yl)amino)-1-(2,2,2-trifluoroethyl)-1H-indol-2-yl) prop-2-yn-1-yl)amino)-3-methoxy-N-methylbenzamide
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-N-hydroxy-3-methoxybenzamide (racemic)
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-N-(2-hydroxy-3-methoxypropyl)-3-methoxy benzamide
5-((3-(2,2,2-trifluoroethyl)-7-(((Z)-3-fluoro-1-methylpiperidin-4-yl)amino)benzo[b]thiophen-2-yl) prop-2-yn-1-yl)amino)-4-methoxy-N-methylpicolinamide (racemic)
5-((3-(3-(2,2,2-trifluoroethyl)-7-(((Z)-3-fluoro-1-methylpiperidin-4-yl)amino)benzo[b]thiophen-2-yl) prop-2-yn-1-yl)amino)-4-methoxypicolinamide (racemic)
5-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-6-methoxy-N-methylpicolinamide (racemic)
5-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-6-methoxypicolinamide (racemic)
(3-(difluoromethoxy)-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide (racemic)
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-(trifluoromethoxy)phenyl)dimethylphosphine oxide (racemic)
diethyl(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-(fluoromethoxy)phenyl)phosphine oxide
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl) (piperidin-1-yl) methanone (racemic)
N-(3-fluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxybenzamide
4-((3-(7-((3-fluoro-1-methylpiperidin-4-yl)amino)-3-(2,2,2-trifluoroethyl)benzo[b]thiophen-2-yl) prop-2-yn-1-yl)amino)-3-methoxy-N-(2-methyltetrahydro-2H-pyran-4-yl)benzamide
(4-((3-(7-((((2R,7aS)-2-fluorotetrahydro-1H-pyrrolizin-7a (5H)-yl)methyl)amino)-3-(2,2,2-trifluoroethyl)benzo[b]thiophen-2-yl) prop-2-yn-1-yl)amino)-3-methoxyphenyl)dimethylphosphine oxide
(3-methoxy-4-((3-(7-((1-methylpiperidin-4-yl)amino)-3-(2,2,2-trifluoroethyl)thieno[3,2-b]pyridin-2-yl) prop-2-yn-1-yl)amino)phenyl)dimethylphosphine oxide
(Z)-(4-((3-(7-((3-fluoro-1′-methy]-[1,4′-bipiperidin]-4-yl)amino)-3-(2,2,2-trifluoroethyl)benzo[b]thiophen-2-yl) prop-2-yn-1-yl)amino)-3-methoxy phenyl)dimethylphosphine oxide (racemic)
(Z)-(4-((3-(7-((3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic)
(4-((3-(7-(((Z)-1-cyclopropyl-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic)
(4-((3-(7-(((Z)-3-fluoro-1-(oxetan-3-yl) piperidin-4-yl)amino)-3-(2,2,2-trifluoroethyl)benzo[b]thiophen-2-yl) prop-2-yn-1-yl)amino)-3-methoxyphenyl)dimethylphosphine oxide
4-(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)-1-methyl-1,4-azaphosphinane4-oxide (racemic)
5-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-6-methoxy-2,3-dihydrobenzo[d]isothiazole1,1-dioxide (racemic)
N-(2-(3-((2-methoxy-4-(methylsulfonyl)phenyl)amino)prop-1-yn-1-yl)-3-(2,2,2-trifluoroethyl)benzo[b]thiophen-7-yl)-3,7-diazabicyclo[3.3.1]nonan-9-amine
N-cyclopropyl-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxy benzamide
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-(2-methoxyethoxy)-N-methylbenzamide (racemic)
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-(2-(2-methoxyethoxy) ethoxy)-N-methylbenzamide (racemic)
(3-chloro-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphineoxide (racemic)
(3-fluoro-4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxy-N-(2-methoxyethyl)benzamide
(4-((3-(7-(((Z)-3-fluoro-1-isopropylpiperidin-4-yl)amino)-3-(2,2,2-trifluoroethyl)benzo[b]thiophen-2-yl) prop-2-yn-1-yl)amino)-3-methoxy phenyl)dimethylphosphine oxide (racemic)
(Z)—N-[2-[3-(4-dimethylphosphoryl-2-methoxy-anilino) prop-1-ynyl]-3-(2,2,2-trifluoroethyl)benzothiophen-7-yl]-3-fluoro-1-(trifluoromethyl) piperidin-4-amine
(4-((3-(7-(((Z)-3-fluoro-1-(2-methoxyethyl) piperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic)
(3-methoxy-4-((3-(7-((5-methyl-5-azaspiro[2.5]octan-8-yl)amino)-3-(2,2,2-trifluoroethyl)benzo[b]thiophen-2-yl) prop-2-yn-1-yl)amino)phenyl)dimethylphosphine oxide
4-((2-(3-((4-(dimethylphosphoryl)-2-methoxyphenyl)amino) prop-1-yn-1-yl)-3-(2,2,2-trifluoroethyl)benzo[b]thiophen-7-yl)amino)-1-methylpiperidine-3-carbonitrile
(2,5-difluoro-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide (racemic)
(5-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-4-methoxypyridin-2-yl)dimethylphosphine oxide
(Z)—N-[2-[3-(4-dimethylphosphoryl-2-methoxy-anilino) prop-1-ynyl]-3-(2,2,2-trifluoroethyl)benzothiophen-7-yl]-1-(1,1-dioxothian-4-yl)-3-fluoro-piperidin-4-amine
N-(3,3-difluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxy benzamide
3-cyano-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-N-methylbenzamide (racemic)
4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxy-N-(2-(2-methoxyethoxy)ethyl)benzamide (racemic)
1-[(Z)-4-[[2-[3-(4-dimethylphosphoryl-2-methoxy-anilino) prop-1-ynyl]-3-(2,2,2-trifluoroethyl)benzothiophen-7-yl]amino]-3-fluoro-1-piperidyl]-3-methoxy-propan-2-ol
(Z)—N-[2-[3-(4-dimethylphosphoryl-2-methoxy-anilino) prop-1-ynyl]-3-(2,2,2-trifluoroethyl)benzothiophen-7-yl]-3-fluoro-1-(trifluoromethyl) piperidin-4-amine
dicyclopropyl(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)phosphine oxide (racemic)
(4-((3-(7-(((Z)-1-ethyl-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic)
(4-((3-(7-(((Z)-3-fluoro-1-isobutylpiperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic)
(4-((3-(7-(((Z)-1-(cyclopropylmethyl)-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((Z)-1-cyclobutyl-3-fluoropiperidin-4-yl)amino)-3-(2,2,2-trifluoroethyl)benzo[b]thiophen-2-yl) prop-2-yn-1-yl)amino)-3-methoxy phenyl)dimethylphosphine oxide (racemic)
(4-((3-(7-(((Z)-1-cyclopentyl-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(trifluoromethyl)phenyl)dimethylphosphine oxide
(3-cyclopropoxy-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide
2-(2-(3-((4-(dimethylphosphoryl)-2-methoxyphenyl)amino) prop-1-yn-1-yl)-7-(((Z)-3-fluoro-1-methylpiperidin-4-yl)amino)benzo[b]thiophen-3-yl)acetonitrile (racemic)
2-(2-(3-((4-(dimethylphosphoryl)-2-methoxyphenyl)amino) prop-1-yn-1-yl)-7-(((E)-3-fluoro-1-methylpiperidin-4-yl)amino)benzo[b]thiophen-3-yl)acetonitrile (racemic)
2-(2-(3-((4-(dimethylphosphoryl)-2-methoxyphenyl)amino) prop-1-yn-1-yl)-7-(((Z)-3-fluoro-1-methylpiperidin-4-yl)amino)benzo[b]thiophen-3-yl) acrylonitrile (racemic)
2-(2-(3-((4-(dimethylphosphoryl)-2-methoxyphenyl)amino) prop-1-yn-1-yl)-7-(((E)-3-fluoro-1-methylpiperidin-4-yl)amino)benzo[b]thiophen-3-yl) acrylonitrile (racemic)
2-dimethylphosphoryl-5-[3-[7-[((Z)-3-fluoro-1-methyl-4-piperidyl)amino]-3-(2,2,2-trifluoroethyl)benzothiophen-2-yl]prop-2-ynylamino]-4-methoxy-N-methyl-benzamide (racemic)
4-((3-(4-(((3S,4R)-3-fluoro-1-methylpiperidin-4-yl)amino)-1-(2,2,2-trifluoroethyl)-1H-indol-2-yl) prop-2-yn-1-yl)amino)-3-methoxy-N-methylbenzamide
4-((3-(4-(((3R,4S)-3-fluoro-1-methylpiperidin-4-yl)amino)-1-(2,2,2-trifluoroethyl)-1H-indol-2-yl) prop-2-yn-1-yl)amino)-3-methoxy-N-methylbenzamide
4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)-3-methoxy-N-methylbenzamide
4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxy-N-methylbenzamide
(4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxy phenyl)dimethylphosphine oxide
(4-((3-(7-(((Z)-3-fluoro-1-methylpiperidin-4-yl)amino)-3-(thiazol-5-yl)benzo[b]thiophen-2-yl) prop-2-yn-1-yl)amino)-3-methoxyphenyl)dimethylphosphine oxide (racemic)
((4-((3-(7-(((E)-3-fluoro-1-methylpiperidin-4-yl)amino)-3-(thiazol-5-yl)benzo[b]thiophen-2-yl) prop-2-yn-1-yl)amino)-3-methoxyphenyl)dimethylphosphine oxide (racemic)
1-(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)phospholane1-oxide
(4-((3-(7-(((Z)-3-fluoro-1-methylpiperidin-4-yl)amino)-3-(2,2,2-trifluoroethyl)benzo[b]thiophen-2-yl) prop-2-yn-1-yl)oxy)-3-methoxyphenyl)dimethylphosphine oxide (racemic)
(3-methoxy-4-((3-(7-((3-methyl-3-azabicyclo[3.2.1]octan-8-yl)amino)-3-(2,2,2-trifluoroethyl)benzo[b]thiophen-2-yl) prop-2-yn-1-yl)amino)phenyl)dimethylphosphine oxide
(4-((3-(7-(((3S,4R)-1,3-dimethylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3R,4S)-1,3-dimethylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3S,4S)-1,3-dimethylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3R,4R)-1,3-dimethylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
1-(tert-butyl)-N-(3-(7-(((Z)-3-fluoro-1-methylpiperidin-4-yl)amino)-3-(2,2,2-trifluoroethyl)benzo[b]thiophen-2-yl) prop-2-yn-1-yl)-1H-pyrazole-4-carboxamide (racemic)
1-tert-butyl-N-[3-[7-[((Z)-3-fluoro-1-methyl-4-piperidyl)amino]-3-(2,2,2-trifluoroethyl)benzothiophen-2-yl]prop-2-ynyl]pyrrole-3-carboxamide (racemic)
(3-methoxy-4-((3-(3-(2,2,2-trifluoroethyl)-7-(((Z)-1,3,5-trimethylpiperidin-4-yl)amino)benzo[b]thiophen-2-yl) prop-2-yn-1-yl)amino)phenyl)dimethylphosphine oxide
(3-methoxy-4-((3-(3-(2,2,2-trifluoroethyl)-7-(((E)-1,3,5-trimethylpiperidin-4-yl)amino)benzo[b]thiophen-2-yl) prop-2-yn-1-yl)amino)phenyl)dimethylphosphine oxide
(4-((3-(7-(((3S,4R)-1-ethyl-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3R,4S)-1-ethyl-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)-3-(trifluoromethoxy)phenyl)dimethylphosphine oxide
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(trifluoromethoxy)phenyl)dimethylphosphine oxide
(4-((3-(7-(((E)-3-fluoro-1-methylpiperidin-4-yl)amino)-3-(4-fluorophenyl)benzo[b]thiophen-2-yl) prop-2-yn-1-yl)amino)-3-methoxyphenyl)dimethylphosphine oxide
dicyclopropyl(4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)phosphine oxide
dicyclopropyl(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)phosphine oxide
(4-((3-(7-(((Z)-3-fluoro-1-methylpiperidin-4-yl)amino)-3-(4-fluorophenyl)benzo[b]thiophen-2-yl) prop-2-yn-1-yl)amino)-3-methoxyphenyl)dimethylphosphine oxide
(3-methoxy-4-((3-(3-(2,2,2-trifluoroethyl)-7-((1,2,6-trimethylpiperidin-4-yl)amino)benzo[b]thiophen-2-yl) prop-2-yn-1-yl)amino)phenyl)dimethylphosphine oxide
(3R,4S)-3-chloro-N-[2-[3-(4-dimethylphosphoryl-2-methoxy-anilino) prop-1-ynyl]-3-(2,2,2-trifluoroethyl)benzothiophen-7-yl]-1-methyl-piperidin-4-amine
(3S,4R)-3-chloro-N-[2-[3-(4-dimethylphosphoryl-2-methoxy-anilino) prop-1-ynyl]-3-(2,2,2-trifluoroethyl)benzothiophen-7-yl]-1-methyl-piperidin-4-amine
(4-((3-(7-(((3S,4R)-3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)-3-methoxy phenyl)dimethylphosphine oxide
(4-((3-(7-(((3R,4S)-3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)-3-methoxyphenyl)dimethylphosphine oxide
cyclopropyl(4-(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)-4-oxido-1,4-azaphosphinan-1-yl) methanone
(3-cyclopropoxy-4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide
(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-2-methoxyphenyl)dimethylphosphine oxide (racemic)
(7-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-2,3-dihydrobenzofuran-4-yl)dimethylphosphine oxide
(5-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-6-methoxypyridin-2-yl)dimethylphosphine oxide
(3-methoxy-4-((3-(7-((1-(tetrahydro-2H-pyran-4-yl) piperidin-4-yl)amino)-3-(2,2,2-trifluoroethyl)benzo[b]thiophen-2-yl) prop-2-yn-1-yl)amino)phenyl)dimethylphosphine oxide
diethyl(3-methoxy-4-((3-(7-((1-(tetrahydro-2H-pyran-4-yl) piperidin-4-yl)amino)-3-(2,2,2-trifluoroethyl)benzo[b]thiophen-2-yl) prop-2-yn-1-yl)amino)phenyl)phosphineoxide
diethyl(4-((3-(7-(((3S,4R)-3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)-3-methoxyphenyl)phosphine oxide
(4-((3-(7-(((3S,4R)-3-fluoro-1-(tetrahydrofuran-3-yl) piperidin-4-yl)amino)-3-(2,2,2-trifluoroethyl)benzo[b]thiophen-2-yl) prop-2-yn-1-yl)amino)-3-methoxyphenyl)dimethylphosphine oxide
2-dimethylphosphoryl-5-[3-[7-[[(3S,4R)-3-fluoro-1-tetrahydropyran-4-yl-4-piperidyl]amino]-3-(2,2,2-trifluoroethyl)benzothiophen-2-yl]prop-2-ynylamino]-4-methoxy-N-methyl-benzamide
(3-cyclopropoxy-4-((3-(7-(((3S,4R)-3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)phenyl)dimethylphosphine oxide
(2-fluoro-5-((3-(7-((3-fluoro-1-methylpiperidin-4-yl)amino)-3-(2,2,2-trifluoroethyl)benzo[b]thiophen-2-yl) prop-2-yn-1-yl)amino)-4-methoxyphenyl)dimethylphosphine oxide
(7-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzo[d][1,3]dioxol-4-yl)dimethylphosphine oxide
(2,2-difluoro-7-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzo[d][1,3]dioxol-4-yl)dimethylphosphine oxide
5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)oxy)benzamide
(3-methoxy-4-((3-(7-(((Z)-1-methyl-3-(trifluoromethyl) piperidin-4-yl)amino)-3-(2,2,2-trifluoroethyl)benzo[b]thiophen-2-yl) prop-2-yn-1-yl)amino)phenyl)dimethylphosphine oxide (racemic)
(3-methoxy-4-((3-(7-(((E)-1-methyl-3-(trifluoromethyl) piperidin-4-yl)amino)-3-(2,2,2-trifluoroethyl)benzo[b]thiophen-2-yl) prop-2-yn-1-yl)amino)phenyl)dimethylphosphine oxide (racemic)
(2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-5-(methylsulfonyl)phenyl)dimethylphosphine oxide
(3S,4R)-3-fluoro-N-(2-(3-((2-methoxy-4-(1-methyl-1H-1,2,4-triazol-3-yl)phenyl)amino)prop-1-yn-1-yl)-3-(2,2,2-trifluoroethyl)benzo[b]thiophen-7-yl)-1-methylpiperidin-4-amine
(3S,4R)-3-fluoro-N-(2-(3-((4-(5-fluoropyrimidin-2-yl)-2-methoxyphenyl)amino) prop-1-yn-1-yl)-3-(2,2,2-trifluoroethyl)benzo[b]thiophen-7-yl)-1-methylpiperidin-4-amine
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(methoxy-d₃) phenyl)dimethylphosphine oxide
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(methylsulfonyl)phenyl)dimethylphosphine oxide
(5-((3-(7-(((3S,4R)-3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)-6-methoxypyridin-2-yl)dimethylphosphine oxide
(4-((3-(7-(((3S,4R)-1-(8-oxabicyclo[3.2.1]octan-3-yl)-3-fluoropiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-(((3S,4R)-3-fluoro-1-(2,2,6,6-tetramethyltetrahydro-2H-pyran-4-yl) piperidin-4-yl)amino)-3-(2,2,2-trifluoroethyl)benzo[b]thiophen-2-yl) prop-2-yn-1-yl)amino)-3-methoxy phenyl)dimethylphosphine oxide
5-((3-(7-((3-fluoro-1-methylpiperidin-4-yl)amino)-3-(2,2,2-trifluoroethyl)benzo[b]thiophen-2-yl) prop-2-yn-1-yl)amino)-6-methoxy-N-methylpicolinamide
N-(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)-N-methylmethanesulfonamide
(4-((3-(7-(((3S,4R)-3-fluoro-1-(tetrahydro-2H-pyran-4-yl) piperidin-4-yl)amino)-3-(2,2,2-trifluoroethyl)benzo[b]thiophen-2-yl) prop-2-yn-1-yl)amino)-3-(methoxy-d₃) phenyl)dimethylphosphine oxide
(4-((3-(7-(((3S,4R)-3-fluoro-1-(oxepan-3-yl) piperidin-4-yl)amino)-3-(2,2,2-trifluoroethyl)benzo[b]thiophen-2-yl) prop-2-yn-1-yl)amino)-3-methoxyphenyl)dimethylphosphine oxide
N-(4-(dimethylphosphoryl)-2-methoxyphenyl)-3-(7-(((3S,4R)-3-fluoro-1-methylpiperidin-4-yl)amino)-3-(2,2,2-trifluoroethyl)benzo[b]thiophen-2-yl)propiolamide
(7-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-2,3-dihydrobenzofuran-4-yl)dimethylphosphine oxide
(4-((3-(7-((3-fluoro-1-methylpiperidin-4-yl)amino)-3-(2,2,2-trifluoro-1-hydroxyethyl)benzo[b]thiophen-2-yl) prop-2-yn-1-yl)amino)-3-methoxy phenyl)dimethylphosphine oxide (racemic)
(3-methoxy-4-((3-(7-((2-methyl-2-azaspiro[3.3]heptan-6-yl)amino)-3-(2,2,2-trifluoroethyl)benzo[b]thiophen-2-yl) prop-2-yn-1-yl)amino)phenyl)dimethylphosphine oxide
4-(4-((3-(7-((3-fluoro-1-methylpiperidin-4-yl)amino)-3-(2,2,2-trifluoroethyl)benzo[b]thiophen-2-yl) prop-2-yn-1-yl)amino)-3-methoxyphenyl) morpholin-3-one
(8-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-2,3-dihydrobenzo[b][1,4]dioxin-5-yl)dimethylphosphine oxide
(2-fluoro-4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)dimethylphosphine oxide
(4-((3-(7-((3-oxaspiro[5.5]undecan-9-yl)amino)-3-(2,2,2-trifluoroethyl)benzo[b]thiophen-2-yl) prop-2-yn-1-yl)amino)-3-methoxyphenyl)dimethylphosphineoxide
(3-methoxy-4-((3-(7-(((1S,5R)-7-methyl-3-oxa-7-azabicyclo[3.3.1]nonan-9-yl)amino)-3-(2,2,2-trifluoroethyl)benzo[b]thiophen-2-yl) prop-2-yn-1-yl)amino)phenyl)dimethylphosphine oxide
(3-methoxy-4-((3-(7-(((1R,5S)-7-methyl-3-oxa-7-azabicyclo[3.3.1]nonan-9-yl)amino)-3-(2,2,2-trifluoroethyl)benzo[b]thiophen-2-yl) prop-2-yn-1-yl)amino)phenyl)dimethylphosphine oxide
(7-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzofuran-4-yl)dimethylphosphine oxide
5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzamide
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(1H-pyrazol-3-yl)phenyl)dimethylphosphine oxide
2-(2-(3-((4-(dimethylphosphoryl)-2-methoxyphenyl)amino) prop-1-yn-1-yl)-7-(((3S,4R)-3-fluoro-1-methylpiperidin-4-yl)amino)benzo[b]thiophen-3-yl) acrylonitrile
2-(2-(3-((4-(dimethylphosphoryl)-2-methoxyphenyl)amino) prop-1-yn-1-yl)-7-(((3R,4S)-3-fluoro-1-methylpiperidin-4-yl)amino)benzo[b]thiophen-3-yl) acrylonitrile
(5-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-1H-pyrazol-3-yl)dimethylphosphine oxide
(4-((3-(7-((1-(3-fluoropropyl) piperidin-4-yl)amino)-3-(2,2,2-trifluoroethyl)benzo[b]thiophen-2-yl) prop-2-yn-1-yl)amino)-3-methoxyphenyl)dimethylphosphineoxide
(3-fluoro-4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-5-methoxyphenyl)dimethylphosphine oxide
(6-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-5-methoxypyridin-3-yl)dimethylphosphine oxide
(5-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-4-methoxypyrimidin-2-yl)dimethylphosphine oxide
(5-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-6-methoxypyrazin-2-yl)dimethylphosphine oxide
(4-((3-(7-(((3S,4R)-3-fluoro-1-(methyl-d3) piperidin-4-yl)amino)-3-(2,2,2-trifluoroethyl)benzo[b]thiophen-2-yl) prop-2-yn-1-yl)amino)-3-methoxy phenyl)dimethylphosphine oxide
S-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzonitrile
3-(5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)phenyl)-1,2,4-oxadiazol-5 (2H)-one
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(1,2,4-oxadiazol-3-yl)phenyl)dimethylphosphine oxide
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(oxazol-5-yl)phenyl)dimethylphosphine oxide
N-cyclopropyl-5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzamide
5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-N-(tetrahydro-2H-pyran-4-yl)benzamide
5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-N-(oxetan-3-yl)benzamide
2-((3-(7-(((3S,4R)-3-chloro-1-methylpiperidin-4-yl)amino)-3-(2,2,2-trifluoroethyl)benzo[b]thiophen-2-yl) prop-2-yn-1-yl)amino)-5-(dimethylphosphoryl)-N,N-dimethylbenzamide
2-((3-(7-(((3S,4R)-3-chloro-1-methylpiperidin-4-yl)amino)-3-(2,2,2-trifluoroethyl)benzo[b]thiophen-2-yl) prop-2-yn-1-yl)amino)-5-(dimethylphosphoryl)-N-(2-methoxyethyl)benzamide
(7-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)benzo[d]thiazol-4-yl)dimethylphosphine oxide
(2-fluoro-4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-(methylthio)phenyl)dimethylphosphine oxide
2-((3S,4R)-4-((2-(3-((4-(dimethylphosphoryl)-2-methoxyphenyl)amino) prop-1-yn-1-yl)-3-(2,2,2-trifluoroethyl)benzo[b]thiophen-7-yl)amino)-3-fluoropiperidin-1-yl)-N-methylacetamide
2-((3-(7-(((3S,4R)-3-chloro-1-methylpiperidin-4-yl)amino)-3-(2,2,2-trifluoroethyl)benzo[b]thiophen-2-yl) prop-2-yn-1-yl)amino)-5-(dimethylphosphoryl)-N-(2-methoxyethyl)benzamide
6-(dimethylphosphoryl)-3-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino) picolinonitrile
(3-(difluoromethyl)-4-((3-(7-(((3R,4S)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide(S)
(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)(imino)(methyl)-16-sulfanone
(R)-(4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)(imino)(methyl)-16-sulfanone.

In some embodiments, the compound is of the formula:

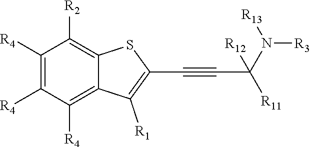

wherein
R₁ is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl;
R² is —NR⁵¹R⁵², —OR⁵³ or —SR⁵⁴;
R³ is selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) R₃ₐ;
each R^{3a} is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(=NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁-6alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more R^{3b}, wherein each R^{3b} is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O) (=NR′)R′, —NR′S(O)₂R′, —NR′S(O)₂N(R′)₂, and —PO(R′)₂;
R⁴ at each occurrence is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂-6alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(=NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl;
R⁵¹, R⁵², R⁵³ and R⁵⁴ are each independently selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl; said -C₁. 6alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, =NR′, —C₁₋₆alkyl-CH(R′)₂, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′) 2, —S(═O)(=NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl;
R¹¹ and R¹² are each independently selected from hydrogen, deuterium, —OH, halogen, —CN, oxo, —C₁₋₆alkyl, —C₁₋₆haloalkyl, —C₁₋₆alkoxy, —NH₂, —NHC₁₋₆alkyl, —N(C₁₋₆alkyl) 2 and 3-6 membered cycloalkyl; wherein said —C₁₋₆alkyl, —C₁₋₆alkoxy and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH—C₁₋₆alkyl, —N(C₁₄ alkyl)₂, and 3-6 membered cycloalkyl;
R¹³ is selected from hydrogen, deuterium, —C₁₋₆alkyl and 3-6 membered cycloalkyl; wherein said —C₁₋₆alkyl and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH₂, —NHC₁₋₆alkyl, and —N(C₁₋₄alkyl)₂;
each R′ at each occurrence is independently selected from hydrogen, deuterium, halogen, —OH, —CN, oxo, —NH₂, —NHC₁₋₆alkyl, —N(C₁₋₆ alkyl) 2, —C₁₋₆alkyl, —C₁₋₆alkylOC₁₋₆alkyl, —C₁₋₆alkyl-NHC₁₋₆alkyl, —C₁₋₆alkyl-N(C₁₋₆alkyl)₂, —C₁₋₆haloalkyl, —OC₁₋₆alkyl, —C₃₋₁₄cycloalkyl, —C₃₋₁₄heterocycloalkyl, —C₂₋₆alkenyl, —C₂₋₆alkynyl, 3-12 membered heterocyclyl, 6-12 membered aryl, and 5-12 membered heteroaryl; wherein said —C₁₋₆alkyl, —OC₁₋₆alkyl, —C₃-14cycloalkyl, —C₃₋₁₄heterocycloalkyl, —C₂₋₆alkenyl, —C₂₋₆alkynyl, 3-12 membered heterocyclyl, 6-12 membered aryl, and 5-12 membered heteroaryl is independently optionally substituted with 1, 2, 3, 4, 5 or 6 substituent s selected from deuterium, halogen, —CN, —C₁₋₃alkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(=NH)C₁₋₃alkyl, —S(═O)(=NC₁₋₃alkyl)C₁₋₃alkyl, and 3-6 membered cycloalkyl; or
two adjacent R′, together with the atoms to which they are attached respectively, can form 3-12 membered cycloalkyl, 3-12 membered cycloalkenyl, 3-12 membered heterocycloalkyl, 3-12 membered heterocycloalkenyl, 3-12 membered heterocyclyl, 6-12 membered aryl, or 5-12 membered heteroaryl, each of which is independently optionally substituted with one or more substituents selected from deuterium, halogen, —CN, —C₁₋₃alkyl, —C₁₋₃haloalkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(=NH)C₁₋₃alkyl, —S(═O)(=NC₁₋₃alkyl) C₁₋₃alkyl, 3-6 membered heterocyclyl, and 3-6 membered cycloalkyl;
said heterocycloalkyl, heterocycloalkenyl, and heteroaryl each independently contains 1, 2, 3, 4 or 5 heteroatoms selected from N, O, P and S;
m is selected from 1, 2, 3, 4, 5 or 6.

In some embodiments, the compound is of the following
N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine
4-((3-(7-((1-methylpiperidin-4-yl)amino)-3-(2,2,2-trifluoroethyl)benzo[b]thiophen-2-yl)prop-2-yn-1-yl)amino)benzenesulfonamide
4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)benzenesulfonamide (racemic)
4-((3-(7-(((3R,4R)-3-fluoro-1-methylpiperidin-4-yl)amino)-3-(2,2,2-trifluoroethyl)benzo[b]thiophen-2-yl)prop-2-yn-1-yl)amino)benzenesulfonamide (racemic)(5b)
1-((3S,4R)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidin-1-yl)-3-methoxypropan-2-ol (racemic)
N-(2-(3-((5-fluoro-2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine(7 3-methoxy-N-methyl-4-((3-(7-((1-methylpiperidin-4-yl)amino)-3-(2,2,2-trifluoroethyl)benzo[b]thiophen-2-yl)prop-2-yn-1-yl)amino)benzamide dimethyl(4-((3-(7-((1-methylpiperidin-4-yl)amino)-3-(2,2,2-trifluoroethyl)benzo[b]thiophen-2-yl)prop-2-yn-1-yl)amino)phenyl)phosphine oxide.
(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(fluoromethoxy)phenyl)dimethylphosphineoxide (racemic)
N-(2-(3-((2-(fluoromethoxy)-4-(methylsulfonyl)phenyl)amino)prop-1-yn-1-yl)-3-(2,2,2-trifluoroethyl)benzo[b]thiophen-7-yl)-1-methylpiperidin-4-amine
N-(2-(3-((2-(2,2-difluoroethoxy)-4-(methylsulfonyl)phenyl)amino)prop-1-yn-1-yl)-3-(2,2,2-trifluoroethyl)benzo[b]thiophen-7-yl)-1-methylpiperidin-4-amine
1-methyl-N-(2-(3-((4-(methylsulfonyl)-2-(2,2,2-trifluoroethoxy)phenyl)amino)prop-1-yn-1-yl)-3-(2,2,2-trifluoroethyl)benzo[b]thiophen-7-yl)piperidin-4-amine
N-[2-[3-[2-(2-methoxyethoxy)-4-methylsulfonyl-anilino]prop-1-ynyl]-3-(2,2,2-trifluoroethyl)benzothiophen-7-yl]-1-methyl-piperidin-4-amine
(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphineoxide (racemic)
N1-(2-(3-((2-methoxy-4-(methylsulfonyl)phenyl)amino)prop-1-yn-1-yl)-3-(2,2,2-trifluoroethyl)benzo[b]thiophen-7-yl)-N4,N4-dimethylcyclohexane-1,4-diamine
Trans-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
Trans-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
cis-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine
Trans-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol)
cis-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol)
N-[2-[3-(2-methoxy-4-methylsulfonyl-anilino)prop-1-ynyl]-3-(2,2,2-trifluoroethyl)benzothiophen-7-yl]-1-(3-methoxypropyl)piperidin-4-amine
3-methoxy-N,N-dimethyl-4-((3-(7-((1-methylpiperidin-4-yl)amino)-3-(2,2,2-trifluoroethyl)benzo[b]thiophen-2-yl)prop-2-yn-1-yl)amino)benzenesulfonamide(21
N-(2-(3-((2-methoxy-4-(morpholinosulfonyl)phenyl)amino)prop-1-yn-1-yl)-3-(2,2,2-trifluoroethyl)benzo[b]thiophen-7-yl)-1-methylpiperidin-4-amine
3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide
N-(2,3-dihydroxypropyl)-3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide
N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.1]octan-8-amine
N-(2-(3-((2-methoxy-4-(methylsulfonyl)phenyl)amino)prop-1-yn-1-yl)-3-(2,2,2-trifluoroethyl)benzo[b]thiophen-7-yl)-8-methyl-8-azabicyclo[3.2.1]octan-3-amine
N-(2-(3-((2-methoxy-4-(methylsulfonyl)phenyl)amino)prop-1-yn-1-yl)-3-(2,2,2-trifluoroethyl)benzo[b]thiophen-7-yl)-2-methyl-2-azabicyclo[2.2.1]heptan-5-amine
N-(2-(3-((2-methoxy-4-(methylsulfonyl)phenyl)amino)prop-1-yn-1-yl)-3-(2,2,2-trifluoroethyl)benzo[b]thiophen-7-yl)-2-azabicyclo[2.2.1]heptan-5-amine
N-(3-(7-((1-methylpiperidin-4-yl)amino)-3-(2,2,2-trifluoroethyl)benzo[b]thiophen-2-yl)prop-2-yn-1-yl)-1H-indol-7-amine
N-(3-(7-((1-methylpiperidin-4-yl)amino)-3-(2,2,2-trifluoroethyl)benzo[b]thiophen-2-yl)prop-2-yn-1-yl)-3-(piperidin-4-yl)-1H-indol-7-amine
N-(3-(7-((1-methylpiperidin-4-yl)amino)-3-(2,2,2-trifluoroethyl)benzo[b]thiophen-2-yl)prop-2-yn-1-yl)-4-(methylsulfonyl)-1H-indol-7-amine
N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.0]heptan-6-amine
N-(2-(3-((2-methoxy-4-(methylsulfonyl)phenyl)amino)prop-1-yn-1-yl)-3-(2,2,2-trifluoroethyl)benzo[b]thiophen-7-yl)-7-methyl-7-azaspiro[3.5]nonan-2-amine
3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine
diethyl(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (racemic)
1-(2-fluoroethyl)-N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine(36
N-(2-(3-((2-methoxy-4-(methylsulfonyl)phenyl)amino)prop-1-yn-1-yl)-3-(2,2,2-trifluoroethyl)benzo[b]thiophen-7-yl)-1-(2,2,2-trifluoroethyl)piperidin-4-amine
N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine
Cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine
trans-N-((1R,4R)-4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine
4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxybenzenesulfonamide (racemic)
4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide (racemic)
(3S,4R)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-N,1-dimethylpiperidin-4-amine(racemic)
(4-((3-(7-((3,3-difluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide
(R)-4-((4-(7-((1-methylpiperidin-4-yl)amino)-3-(2,2,2-trifluoroethyl)benzo[b]thiophen-2-yl)but-3-yn-2-yl)amino)benzenesulfonamide
(S)-4-((4-(7-((1-methylpiperidin-4-yl)amino)-3-(2,2,2-trifluoroethyl)benzo[b]thiophen-2-yl)but-3-yn-2-yl)amino)benzenesulfonamide
4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (racemic)
4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-(1-methylpiperidin-4-yl)benzamide (racemic)
(1,1-dioxidothiomorpholino)(4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)methanone (racemic)
(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(4-methylpiperazin-1-yl)methanone (racemic)
(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(morpholino)methanone (racemic)
N-(4-fluoro-2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine
(2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-methoxyphenyl)dimethylphosphineoxide (racemic)
(S)-(5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyridin-2-yl)(imino)(methyl)-16-sulfanone
(R)-(5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyridin-2-yl)(imino)(methyl)-16-sulfanone (S)-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(oxetan-3-yl)-16-sulfanone
(R)-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(oxetan-3-yl)-16-sulfanone

In some embodiments, the compound is of the formula:

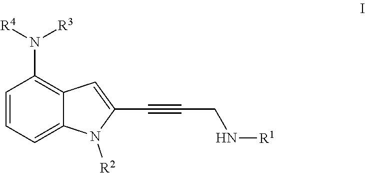

wherein:
R¹ is selected from aryl, heteroaryl, and heterocyclyl, each of which is independently unsubstituted or substituted with one, two or three substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and an ester group;
R² is selected from hydrogen, —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently unsubstituted or substituted with one, two or three substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and an ester group;
R³ is H;
R⁴ is heterocyclyl substituted with one, two or three substituents selected from a halogen group, a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and an ester group;
each R²¹ and R²² is independently hydrido, alkyl, alkenyl, alkynyl, aryl, heteroaryl, or heterocyclyl, each of which is independently substituted or unsubstituted; and

wherein the compound comprises at least one deuterio group;

or a pharmaceutically-acceptable salt thereof.

In some embodiments, the compound is of the formula:

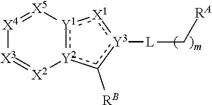

wherein:
X¹ is CR¹ or N;
R¹ is hydrogen, halogen, cyano, —OR⁴, —NR⁴R⁵, —C(═O)R⁴, —OC(═O)R⁴, —C(═O)OR⁴, —C(═O)NR⁴R⁵, —SR⁴, —S(═O)R⁴, —S(O)₂R⁴, —NR⁴C(═O)R, —R⁴C(═O)R′, optionally substituted C1-C6 alkyl, optionally substituted C2-C6 alkenyl, optionally substituted C2-C26 alkynyl, optionally substituted C3-C10 cycloalkyl, optionally substituted phenyl, optionally substituted 4-12 membered heterocyclyl, or optionally substituted 5-10 membered heteroaryl;
each of X², X³, X¹, and X⁶ are CH N, CR² or CR³, wherein two or more of X², X³, X⁴, and X are independently CH, CR², or CR³:
each of Y¹, Y², and Y³ are C or N, wherein one of Y¹, Y², and Y³ is N;
R^{A} is hydrogen, —OR⁶, —NR⁶R⁷, —C(O)R⁶, —R⁶C(O)R⁷, —OC(═O)R⁶, —OC(═O)NR⁶R⁷, —C(═O)OR⁶, —NR⁶C(═O)OR′, —C(═O)NR⁶R⁷, —SR⁶, —S(═O)R⁶, —S(O)₂R, —S(O)₂NR⁶R⁷, —NR⁶S(O)₂R⁷, —NR⁶C(═O)R⁷, —NR⁶C(═O)NR⁷R⁸, —SiR⁶R⁷R⁸, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
R^{B} is halogen, cyano, hydroxyl, —NR⁸R⁹, —OR′, —C(═O)NR⁸R⁹, —C(═O)R′, —C(═O)OR⁸, —NR⁸C(═O)OR⁹, —OC(═O)R⁸, —OC(═O)NR⁸R⁹, —C(═O)NR⁸R⁹, —NR⁸C(═O)R⁹, —NR⁷C(═O)NR⁸R⁹, —SR⁸, —S(═O)R⁸, —S(O)₂R⁸, —S(O)₂NR⁸, —NR⁸S(O)₂R⁹, —R⁸C(═O)R⁹, —NR⁸C(═O)R⁹, —NR⁷C(═O)NR⁸R⁹, optionally substituted C1-C6 alkyl, C1-C6 haloalkyl, optionally substituted C2-C6 alkenyl, optionally substituted C2-C6 alkynyl, optionally substituted C3-C10 cycloalkyl, optionally substituted phenyl, optionally substituted 3-12 membered heterocyclyl, or optionally substituted 5-10 membered heteroaryl;
each R² is

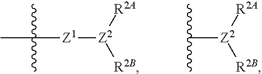

-Z¹—R^{2A}, or —R^{2A};
Z¹ is —C(═O)—, —S(O)₂-optionally substituted C1-C6 alkylene, optionally substituted C2-C6 alkenylene, optionally substituted C2-C6 alkynylene, or an optionally substituted C3-C4 cycloalkylene;
Z² is CR^{2C}, N, or O,
wherein when Z² is O, R^{2B} is absent;
R^{2A} and R^{2B} are independently hydrogen, —C(═O)R¹⁰, —C(═O)OR¹⁰, —C(═O)NR¹⁰R¹¹, —S(═O)R¹⁰, —S(O)₂R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl; or R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl; or Z² is O and R^{2B} is absent
R^{2C} is hydrogen, halogen, or C1-C6 alkyl;
each R³ is independently halogen, cyano, —NR¹²R¹³, —OR¹², —C(═O)NR¹²R¹³, —C(═O)R¹², —C(═O)OR¹², —OC(═O)R¹², —NR¹²(C═O)NRR¹⁴, —SR¹², —S(═O)R¹², —S(O)₂R¹², —S(O)₂NR¹²R¹³, —NR¹²S(O)₂NR¹³R¹⁴, —R¹²C(═O)R¹³, —NR¹²C(═O)R¹³, optionally substituted C1-C6 alkyl, optionally substituted C2-C6 alkenyl, optionally substituted C2-C6 alkynyl, optionally substituted C₃-C₆ cycloalkyl, optionally substituted phenyl, optionally substituted 4-6 membered heterocyclyl, or optionally substituted 5-6 membered heteroaryl;
L is an optionally substituted 4-6 membered heterocyclylene or an optionally substituted 5-6 membered heteroarylene;
m is 0, 1, or 2; and
each R⁴, R⁵, R⁶, R⁷, R⁸, R⁹, R¹⁰, R¹¹, R¹², R¹³, and R¹⁴ are independently hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, the compound is:
N-((3-(8-(((3S,4R)-3-fluoro-1-methylpiperidin-4-yl)amino)-3-vinylimidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-vinyl-2H-indazol-2-yl)-1,2,4-oxadiazol-5-yl)methyl)cyclopropanecarboxamide;
N-((3-(7-(((3S,4R)-3-fluoro-1-methylpiperidin-4-yl)amino)-3-vinylpyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-vinyl-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-vinyl-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-vinylimidazo[1,2-a]pyridin-2-yl)-1,2,4-oxadiazol-5-yl)methyl)-1H-pyrazole-4-carboxamide;
1-(tert-butyl)-N-((3-(8-(((3 S,4R)-3-fluoro-1-methylpiperidin-4-yl)amino)-3-vinylimidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-vinylpyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-vinylpyrazolo[1,5-a]pyridin-2-yl)-1,2,4-oxadiazol-5-yl)methyl)-1H-pyrrole-3-carboxamide;
N-((5-(7-(((3S,4R)-3-fluoro-1-methylpiperidin-4-yl)amino)-3-vinyl-2H-indazol-2-yl)-1,3,4-thiadiazol-2-yl)methyl)cyclopropanecarboxamide;
N-((5-(8-(((3 S,4R)-3-fluoro-1-methylpiperidin-4-yl)amino)-3-vinylimidazo[1,2-a]pyridin-2-yl)-1,3,4-thiadiazol-2-yl)methyl)cyclopropanecarboxamide;
N-((5-(7-(((3S,4R)-3-fluoro-1-methylpiperidin-4-yl)amino)-3-vinyl-1-pyrazolo[1,5-a]pyridin-2-yl)-1,3,4-thiadiazol-2-yl)methyl)cyclopropanecarboxamide;
1-(tert-butyl)-N-((5-(7-(((3S,4R)-3-fluoro-1-methylpiperidin-4-yl)amino)-3-vinyl-2H-indazol-2-yl)-1,3,4-thiadiazol-2-yl)methyl)-1H-pyrazole-4-carboxamide;
1-(tert-butyl)-N-((5-(7-(((3 S,4R)-3-fluoro-1-methylpiperidin-4-yl)amino)-3-vinyl-2H-indazol-2-yl)-1,3,4-thiadiazol-2-yl)methyl)-1H-pyrrole-3-carboxamide;
1-(tert-butyl)-N-((5-(8-(((3S,4R)-3-fluoro-1-methylpiperidin-4-yl)amino)-3-vinylimidazo[1,2-a]pyridin-2-yl)-1,3,4-thiadiazol-2-yl)methyl)-1H-pyrazole-4-carboxamide;
1-(tert-butyl)-N-((5-(8-(((3 S,4R)-3-fluoro-1-methylpiperidin-4-yl)amino)-3-vinylimidazo[1,2-a]pyridin-2-yl)-1,3,4-thiadiazol-2-yl)methyl)-1H-pyrrole-3-carboxamide;
1-(tert-butyl)-N-((5-(7-(((3S,4R)-3-fluoro-1-methylpiperidin-4-yl)amino)-3-vinylpyrazolo[1,5-a]pyridin-2-yl)-1,3,4-thiadiazol-2-yl)methyl)-1H-pyrazole-4-carboxamide;
1-(tert-butyl)-N-((5-(7-(((3S,4R)-3-fluoro-1-methylpiperidin-4-yl)amino)-3-vinylpvrazolo[1,5-a]pyridin-2-yl)-1,3,4-thiadiazol-2-yl)methyl)-1H-pyrrole-3-carboxamide;
N-((3-(5-amino-7-(((3S,4R)-3-fluoro-1-methylpiperidin-4-yl)amino)-3-vinyl-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-vinylimidazo[1,2-a]pyrazin-2-yl)-1,2,4-oxadiazol-5-yl)methyl)cyclopropanecarboxamide;
N-((3-(7-(((3S,4R)-3-fluoro-1-methylpiperidin-4-yl)amino)-3-vinylpyrazolo[1,5-a]pyrazin-2-yl)-1,2,4-oxadiazol-5-yl)methyl)cyclopropanecarboxamide;
N-((3-(5-amino-7-(((3 S,4R)-3-fluoro-1-methylpiperidin-4-yl)amino)-3-vinyl-2H-indazol-2-yl)-1,2,4-oxadiazol-5-yl)methyl)-1-(tert-butyl)-1H-pyrazole-4-carboxamide;
N-((3-(5-amino-7-(((3 S,4R)-3-fluoro-1-methylpiperidin-4-yl)amino)-3-vinyl-2H-indazol-2-yl)-1,2,4-oxadiazol-5-yl)methyl)-1-(tert-butyl)-1H-pyrrole-3-carboxamide;
1-(tert-butyl)-N-((3-(8-(((3S,4R)-3-fluoro-1-methylpiperidin-4-yl)amino)-3-vinylimidazo[1,2-a]pyrazin-2-yl)-1,2,4-oxadiazol-5-yl)methyl)-1H-pyrazole-4-carboxamide;
1-(tert-butyl)-N-((3-(8-(((3 S,4R)-3-fluoro-1-methylpiperidin-4-yl)amino)-3-vinylimidazo[1,2-a]pyrazin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-vinylpyrazolo[1,5-a]pyrazin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-vinylpyrazolo[1,5-a]pyrazin-2-yl)-1,2,4-oxadiazol-5-yl)methyl)-1H-pyrrole-3-carboxamide;
N-((5-(5-amino-7-(((3S,4R)-3-fluoro-1-methylpiperidin-4-yl)amino)-3-vinyl-2H-indazol-2-yl)-1,3,4-thiadiazol-2-yl)methyl)cyclopropanecarboxamide;
N-((5-(8-(((3S,4R)-3-fluoro-1-methylpiperidin-4-yl)amino)-3-vinylimidazo[1,2-a]pyrazin-2-yl)-1,3,4-thiadiazol-2-yl)methyl)cyclopropanecarboxamide;
N-((5-(7-(((3S,4R)-3-fluoro-1-methylpiperidin-4-yl)amino)-3-vinylpyrazolo[1,5-a]pyrazin-2-yl)-1,3,4-thiadiazol-2-yl)methyl)cyclopropanecarboxamide;
N-((5-(5-amino-7-(((3S,4R)-3-fluoro-1-methylpiperidin-4-yl)amino)-3-vinyl -2H-indazol-2-yl)-1,3,4-thiadiazol-2-yl)methyl)-1-(tert-butyl)-1H-pyrazole-4-carboxamide;
N-((5-(5-amino-7-(((3S,4R)-3-fluoro-1-methylpiperidin-4-yl)amino)-3-vinyl-2H-indazol-2-yl)-1,3,4-thiadiazol-2-yl)methyl)-1-(tert-butyl)-1H-pyrrole-3-carboxamide;
1-(tert-butyl)-N-((5-(8-(((3S,4R)-3-fluoro-1-methylpiperidin-4-yl)amino)-3-vinylimidazo[1,2-a]pyrazin-2-yl)-1,3,4-thiadiazol-2-yl)methyl)-1H-pyrazole-4-carboxamide;
1-(tert-butyl)-N-((5-(8-(((3S,4R)-3-fluoro-1-methylpiperidin-4-yl)amino)-3-vinylimidazo[1,2-a]pyrazin-2-yl)-1,3,4-thiadiazol-2-yl)methyl)-1H-pyrrole-3-carboxamide;
1-(tert-butyl)-N-((5-(7-(((3S,4R)-3-fluoro-1-methylpiperidin-4-yl)amino)-3-vinvlpyrazolo[1,5-a]pyrazin-2-yl)-1,3,4-thiadiazol-2-yl)methyl)-1H-pyrazole-4-carboxamide;
1-(tert-butyl)-N-((5-(7-(((3S,4R)-3-fluoro-1-methylpiperidin-4-yl)amino)-3-vinylpyrazolo[1,5-a]pyrazin-2-yl)-1,3,4-thiadiazol-2-yl)methyl)-1H-pyrrole-3-carboxamide;
N-((3-(5-fluoro-7-(((3S,4R)-3-fluoro-1-methylpiperidin-4-yl)amino)-3-vinyl-2H-indazol-2-yl)-1,2,4-oxadiazol-5-yl)methyl)cyclopropanecarboxamide;
1-(tert-butyl)-N-((3-(5-fluoro-7-(((3S,4R)-3-fluoro-1-methylpiperidin-4-yl)amino)-3-vinyl-2H-indazol-2-yl)-1,2,4-oxadiazol-5-yl)methyl)-1H-pyrazole-4-carboxamide;
1-(tert-butyl)-N-((3-(5-fluoro-7-(((3S,4R)-3-fluoro-1-methylpiperidin-4-yl)amino)-3-vinyl-2H-indazol-2-yl)-1,2,4-oxadiazol-5-yl)methyl)-1H-pyrrole-3-carboxamide;
N-((5-(5-fluoro-7-(((3S,4R)-3-fluoro-1-methylpiperidin-4-yl)amino)-3-vinyl-2H-indazol-2-yl)-1,3,4-thiadiazol-2-yl)methyl)cyclopropanecarboxamide;
1-(tert-butyl)-N-((5-(5-fluoro-7-(((3S,4R)-3-fluoro-1-methylpiperidin-4-yl)amino)-3-vinyl-2H-indazol-2-yl)-1,3,4-thiadiazol-2-yl)methyl)-1H-pyrazole-4-carboxamide;
1-(tert-butyl)-N-((5-(5-fluoro-7-(((3S,4R)-3-fluoro-1-methylpiperidin-4-yl)amino)-3-vinyl-2H-indazol-2-yl)-1,3,4-thiadiazol-2-yl)methyl)-1H-pyrrole-3-carboxamide;
N-((3-(7-(((3S,4R)-3-fluoro-1-methylpiperidin-4-yl)amino)-3-(prop-1-en-2-yl)-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-(prop-1-en-2-yl)imidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-(prop-1-en-2-yl)pyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-(prop-1-en-2-yl)-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(prop-en-2-yl)-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-(prop-1-en-2-yl)imidazo[1,2-a]pyridin-2-yl)-1,2,4-oxadiazol-5-yl)methyl)-1H-pyrazole-4-carboxamide;
1-(tert-buty)-N-((3-(8-(((3S,4R)-3-fluoro-1-methylpiperidin-4-yl)amino)-3-(prop-1-en-2-yl)imidazo[1,2-a]pyridin-2-yl)-1,2,4-oxadiazol-5-yl)methyl)-1H-pyrrole-3-carboxamide;
1-(tert-butyl)-N-((3-(7-(((3 S,4R)-3-fluoro-1-methylpiperidin-4-yl)amino)-3-(prop-1-en-2-yl)pyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(prop-1-en-2-yl)pyrazolo[1,5-a]pyridin-2-yl)-1,2,4-oxadiazol-5-yl)methyl)-1H-pyrrole-3-carboxamide;
N-((3-(7-(((3S,4R)-3-fluoro-1-methylpiperidin-4-yl)amino)-3-(1-fluorovinyl)-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-(1-fluorovinyl)imidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-(1-fluorovinyl)pyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-(1-fluorovinyl)-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(1-fluorovinyl)-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-(1-fluorovinyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-(1-fluorovinyl)imidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-(1-fluorovinyl)pyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(1-fluorovinyl)pyrazolo[1,5-a]pyridin-2-yl)-1,2,4-oxadiazol-5-yl)methyl)-1H-pyrrole-3-carboxamide;
N-((3-(7-(((3S,4R)-3-fluoro-1-methylpiperidin-4-yl)amino)-3-((E)-prop-1-en-1-yl)-2H-indazol-2-yl)-1,2,4-oxadiazol-5-yl)methyl)cyclopropanecarboxamide;
N-((3-(8-(((3 S,4R)-3-fluoro-1-methylpiperidin-4-yl)amino)-3-((E)-prop-1-en-1-yl)imidazo[1,2-a]pyridine-2-yl)-1,2,4-oxadiazol-5-yl)methyl)cyclopropanecarboxamide;
N-((3-(7-(((3S,4R)-3-fluoro-1-methylpiperidin-4-yl)amino)-3-((E)-prop-1-en-1-yl)pyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-((E)-prop-1-en-1-yl)-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-((E)-prop-1-en-1-yl)-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-((E)-prop-1-en-1-yl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-((E)-prop-1-en-1-yl)imidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-((E)-prop-1-en-1-yl)pyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-((E)-prop-1-en-1-yl)pyrazolo[1,5-a]pyridin-2-yl)-1,2,4-oxadiazol-5-yl)methyl)-1H-pyrrole-3-carboxamide;
N-((3-(3-(2,2-difluorovinyl)-7-(((3S,4R)-3-fluoro-1-methylpiperidin-4-yl)amino)-2H-indazol-2-yl)-1,2,4-oxadiazol-5-yl)methyl)cyclopropanecarboxamide;
N-((3-(3-(2,2-difluorovinyl)-8-(((3S,4R)-3-fluoro-1-methylpiperidin-4-yl)amino)imidazo[1,2-a]pyridin-2-yl)-1,2,4-oxadiazol-5-yl)methyl)cyclopropanecarboxamide;
N-((3-(3-(2,2-difluorovinyl)-7-(((3S,4R)-3-fluoro-1-methylpiperidin-4-yl)amino)pyrazolol 1,5-a]pyridin-2-yl)-1,2,4-oxadiazol-5-yl)methyl)cyclopropanecarboxamide;
1-(tert-butyl)-N-((3-(3-(2,2-difluorovinyl)-7-(((3S,4R)-3-fluoro-1-methylpiperidin-4-yl)amino)-2H-indazol-2-yl)-1,2,4-oxadiazol-5-yl)methyl)-1H-pyrazole-4-carboxamide;
1-(tert-butyl)-N-((3-(3-(2,2-difluorovinyl)-7-(((3S,4R)-3-fluoro-1-methylpiperidin-4-yl)amino)-2H-indazol-2-yl)-1,2,4-oxadiazol-5-yl)methyl)-1H-pyrrole-3-carboxamide;
1-(tert-butyl)-N-((3-(3-(2,2-difluorovinyl)-8-(((3S,4R)-3-fluoro-1-methylpiperidin-4-yl)amino)imidazo[1,2-a]pyridin-2-yl)-1,2,4-oxadiazol-5-yl)methyl)-1H-pyrazole-4-carboxamide;
1-(tert-butyl)-N-((3-(3-(2,2-difluorovinyl)-8-(((3S,4R)-3-fluoro-1-methylpiperidin-4-yl)amino)imidazo[1,2-a]pyridin-2-yl)-1,2,4-oxadiazol-5-yl)methyl)-1H-pyrrole-3-carboxamide;
1-(tert-butyl)-N-((3-(3-(2,2-difluorovinyl)-7-(((3S,4R)-3-fluoro-1-methylpiperidin-4-yl)amino)pyrazolo[1,5-a]pyridin-2-yl)-1,2,4-oxadiazol-5-yl)methyl)-1H-pyrazole-4-carboxamide;
1-(tert-butyl)-N-((3-(3-(2,2-difluorovinyl)-7-(((3S,4R)-3-fluoro-1-methylpiperidin-4-yl)amino)pyrazolo[1,5-a]pyridin-2-yl)-1,2,4-oxadiazol-5-yl)methyl)-1E[-pyrrole-3-carboxamide;
N-((3-(7-(((3S,4R)-3-fluoro-1-methylpiperidin-4-yl)amino)-3-(1,2,2-trifluorovinyl)-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-(1,2,2-trifluorovinyl)imidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-(1,2,2-trifluorovinyl)pyrazolo[1,5-a]pyridin-2-yl)-1,2,4-oxadiazol-5-yl)methyl)cyclopropanecarboxamide;
2-(5-((((1-(tert-butyl)-1H-pyrazol-4-yl)(12-fluorenylidene)methyl)amino)methyl)-1,2,4-oxadiazol-3-yl)-N-((3S,4R)-3-fluoro-1-methylpiperidin-4-yl)-3-(1,2,2-trifluorovinyl)-2H-indazol-7-amine;
1-(tert-butyl)-N-((3-(7-(((3S,4R)-3-fluoro-1-methylpiperidin-4-yl)amino)-3-(1,2,2-trifluorovinyl)-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-(1,2,2-trifluorovinyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-(1,2,2-trifluorovinyl)imidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-(1,2,2-trifluorovinyl)pyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(1,2,2-trifluorovinyl)pyrazolo[1,5-a]pyridin-2-yl)-1,2,4-oxadiazol-5-yl)methyl)-1H-pyrrole-3-carboxamide;
N-((3-(7-(((3S,4R)-3-fluoro-1-methylpiperidin-4-yl)amino)-3-((R)-oxiran-2-yl)-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-((S)-oxiran-2-yl)imidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-((R)-oxiran-2-yl)pyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-((S)-oxiran-2-yl)-2H-indazol-2-yl)-1,2,4-oxadiazol-5-yl)methyl)-1H-pyrazole-4-carboxamide;
1-(tert-buty)-N-((3-(7-(((3S,4R)-3-fluoro-1-methylpiperidin-4-yl)amino)-3-((S)-oxiran-2-yl)-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-((R)-oxiran-2-yl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-((S)-oxiran-2-yl)imidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-((R)-oxiran-2-yl)pyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-((R)-oxiran-2-yl)pyrazolo[1,5-a]pyridin-2-yl)-1,2,4-oxadiazol-5-yl)methyl)-1H-pyrrole-3-carboxamide;
N-((3-(7-(((3S,4R)-3-fluoro-1-methylpiperidin-4-yl)amino)-3-((S)-oxiran-2-yl)-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-((R)-oxiran-2-yl)imidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-((S)-oxiran-2-yl)pyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-((R)-oxiran-2-yl)-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-((R)-oxiran-2-yl)-2H-indazol-2-yl)-1,2,4-oxadiazol-5-yl)methyl)-1H-pyrrole-3-carboxamide;
1-(tert-butyl)-N-((3-(8-(((3S,4R)-3-fluoro-1-methylpiperidin-4-yl)amino)-3-((S)-oxiran-2-yl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-((R)-oxiran-2-yl)imidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-((S)-oxiran-2-yl)pyrazolo[1,5-a]pyridin-2-yl)-1,2,4-oxadiazol-5-yl)methyl)-1H-pyrrole-3-carboxamide;
N-((3-(6-acrylamido-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1-(tert-butyl)-1H-pyrazole-4-carboxamide;
1-(tert-butyl)-N-((3-(8-(((3S,4R)-3-fluoro-1-methylpiperidin-4-yl)amino)-6-methacrylamido-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(6-((E)-4-(dimethylamino)but-2-enamido)-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-6-((E)-4-(4-methylpiperazin-1-yl)but-2-enamido)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-6-(4-(methylamino)but-2-ynamido)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-6-(2-fluoroacrylamido)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-6-propiolamido-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-6-((E)-4-(methylamino)but-2-enamido)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-6-((E)-4-morpholinobut-2-enamido)-3-(2,2,2-trifluoroethyl)imidazo[1,2-a]pyridin-2-yl)-1,2,4-oxadiazol-5-yl)methyl)-1H-pyrazole-4-carboxamide;
N-((3-(6-(but-2-ynamido)-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1-(tert-butyl)-1H-pyrazole-4-carboxamide;
N-((3-(6-((E)-but-2-enamido)-8-(((3S,4R)-3-fluoro-1-methylpiperidin-4-yl)amino)-3-(2,2,2-trifluoroethyl)imidazo[1,2-a]pyridin-2-yl)-1, 2, 4-oxadiazol-5-yl)methyl)-1-(tert-butyl)-1H-pyrazole-4-carboxamide;
1-(tert-butyl)-N—((3-(6-((E)-4-(3,3-difluoroazetidin-1-yl)but-2-enamido)-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(6-(4-(dimethylamino)but-2-ynamido)-8-(((3 S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide;
N-((3-(7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)cyclopropanecarboxamide;
N-((3-(8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)cyclopropanecarboxamide;
N-((3-(7-(((3S,4R)-3-fluoro-1-methylpiperidin-4-yl)amino)-3-(2,2,2-trifluoroethyl)pyrazolo[1,5-a]pyridin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrrole-3-carboxamide;
1-(tert-butyl)-N-((3-(8-(((3 S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrrole-3-carboxamide;
1-(tert-butyl)-N-((3-(7-(((3S,4R)-3-fluoro-1-methylpiperidin-4-yl)amino)-3-(2,2,2-trifluoroethyl)pyrazolo[1,5-a]pyridin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(2,2,2-trifluoroethyl)pyrazolo[1,5-a]pyridin-2-yl)-1,2,4-oxadiazol-5-yl)methyl)-1H-pyrrole-3-carboxamide;
N-((5-(7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)cyclopropanecarboxamide;
1-(tert-butyl)-N-((5-(7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrazole-4-carboxamide;
1-(tert-butyl)-N-((5-(7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrrole-3-carboxamide;
N-((5-(8-(((3S,4R)-3-fluoro-1-methylpiperidin-4-yl)amino)-3-(2,2,2-trifluoroethyl)imidazol 1,2-a]pyridin-2-yl)-1,3,4-thiadiazol-2-yl)methyl)cyclopropanecarboxamide;
1-(tert-butyl)-N-((5-(8-(((3S,4R)-3-fluoro-1-methylpiperidin-4-yl)amino)-3-(2,2,2-trifluoroethyl)imidazo[1,2-a]pyridin-2-yl)-1,3,4-thiadiazol-2-yl)methyl)-1H-pyrazole-4-carboxamide;
1-(tert-butyl)-N-((5-(8-(((3S,4R)-3-fluoro-1-methylpiperidin-4-yl)amino)-3-(2,2,2-trifluoroethyl)imidazo[1,2-a]pyridin-2-yl)-1,3,4-thiadiazol-2-yl)methyl)-1H-pyrrole-3-carboxamide;
N-((5-(7-(((3S,4R)-3-fluoro-1-methylpiperidin-4-yl)amino)-3-(2,2,2-trifluoroethyl)pyrazolo[1,5-a]pyridin-2-yl)-1,3,4-thiadiazol-2-yl)methyl)cyclopropanecarboxamide;
1-(tert-butyl)-N-((5-(7-(((3S,4R)-3-fluoro-1-methylpiperidin-4-yl)amino)-3-(2,2,2-trifluoroethyl)pyrazolo[1,5-a]pyridin-2-yl)-1,3,4-thiadiazol-2-yl)methyl)-1H-pyrazole-4-carboxamide;
1-(tert-butyl)-N-((5-(7-(((3S,4R)-3-fluoro-1-methylpiperidin-4-yl)amino)-3-(2,2,2-trifluoroethyl)pyrazolo[1,5-a]pyridin-2-yl)-1,3,4-thiadiazol-2-yl)methyl)-1H-pyrrole-3-carboxamide;
N-((3-(5-amino-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)cyclopropanecarboxamide;
N-((3-(5-amino-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1-(tert-butyl)-1H-pyrazole-4-carboxamide;
N-((3-(5-amino-7-(((3S,4R)-3-fluoro-1-methylpiperidin-4-yl)amino)-3-(2, 2, 2-trifluoroethyl)-2H-indazol-2-yl)-1, 2, 4-oxadiazol-5-yl)methyl)-1-(tert-butyl)-1H-pyrrole-3-carboxamide;
N-((3-(8-(((3S,4R)-3-fluoro-1-methylpiperidin-4-yl)amino)-3-(2,2,2-trifluoroethyl)imidazo[1,2-a]pyrazin-2-yl)-1,2,4-oxadiazol-5-yl)methyl)cyclopropanecarboxamide;
1-(tert-butyl)-N-((3-(8-(((3S,4R)-3-fluoro-1-methylpiperidin-4-yl)amino)-3-(2,2,2-trifluoroethyl)imidazo[1,2-a]pyrazin-2-yl)-1,2,4-oxadiazol-5-yl)methyl)-1H-pyrazole-4-carboxamide;
1-(tert-butyl)-N-((3-(8-(((3S,4R)-3-fluoro-1-methylpiperidin-4-yl)amino)-3-(2,2,2-trifluoroethyl)imidazo[1,2-a]pyrazin-2-yl)-1,2,4-oxadiazol-5-yl)methyl)-1H-pyrrole-3-carboxamide;
N-((3-(7-(((3S,4R)-3-fluoro-1-methylpiperidin-4-yl)amino)-3-(2,2,2-trifluoroethyl)pyrazolo[1,5-a]pyrazin-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-(2,2,2-trifluoroethyl)pyrazolo[1,5-a]pyrazin-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(2,2,2-trifluoroethyl)pyrazolo[1,5-a]pyrazin-2-yl)-1,2,4-oxadiazol-5-yl)methyl)-1H-pyrrole-3-carboxamide;
N-((5-(5-amino-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)cyclopropanecarboxamide;
N-((5-(5-amino-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1-(tert-butyl)-1H-pyrrole-3-carboxamide;
N-((5-(8-(((3S,4R)-3-fluoro-1-methylpiperidin-4-yl)amino)-3-(2,2,2-trifluoroethyl)imidazo[1,2-a]pyrazin-2-yl)-1,3,4-thiadiazol-2-yl)methyl)cyclopropanecarboxamide;
1-(tert-butyl)-N-((5-(8-(((3S,4R)-3-fluoro-1-methylpiperidin-4-yl)amino)-3-(2,2,2-trifluoroethyl)imidazo[1,2-a]pyrazin-2-yl)-1,3,4-thiadiazol-2-yl)methyl)-1H-pyrazole-4-carboxamide;
1-(tert-butyl)-N-((5-(8-(((3S,4R)-3-fluoro-1-methylpiperidin-4-yl)amino)-3-(2,2,2-trifluoroethyl)imidazo[1,2-a]pyrazin-2-yl)-1,3,4-thiadiazol-2-yl)methyl)-1H-pyrrole-3-carboxamide;
N-((5-(7-(((3S,4R)-3-fluoro-1-methylpiperidin-4-yl)amino)-3-(2,2,2-trifluoroethyl)pyrazolo[1,5-a]pyrazin-2-yl)-1,3,4-thiadiazol-2-yl)methyl)cyclopropanecarboxamide;
N-((5-(5-amino-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1-(tert-butyl)-1H-pyrazole-4-carboxamide;
1-(tert-butyl)-N-((5-(7-(((3S,4R)-3-fluoro-1-methylpiperidin-4-yl)amino)-3-(2,2,2-trifluoroethyl)pyrazolo[1,5-a]pyrazin-2-yl)-1,3,4-thiadiazol-2-yl)methyl)-1H-pyrazole-4-carboxamide;
1-(tert-butyl)-N-((5-(7-(((3S,4R)-3-fluoro-1-methylpiperidin-4-yl)amino)-3-(2,2,2-trifluoroethyl)pyrazolo[1,5-a]pyrazin-2-yl)-1,3,4-thiadiazol-2-yl)methyl)-1H-pyrrole-3-carboxamide;
N-((3-(5-fluoro-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)cyclopropanecarboxamide;
1-(tert-butyl)-N-((3-(5-fluoro-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrazole-4-carboxamide;
1-(tert-butyl)-N-((3-(5-fluoro-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrrole-3-carboxamide;
N-((5-(5-fluoro-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)cyclopropanecarboxamide;
1-(tert-butyl)-N-((5-(5-fluoro-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrazole-4-carboxamide;
1-(tert-butyl)-N-((5-(5-fluoro-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrrole-3-carboxamide;
N-((3-(7-(((3S,4R)-3-fluoro-1-methylpiperidin-4-yl)amino)-3-(perfluoroethyl)-2H-indazol-2-yl)-1,2,4-oxadiazol-5-yl)methyl)cyclopropanecarboxamide;
1-(tert-butyl)-N-((3-(7-(((3S,4R)-3-fluoro-1-methylpiperidin-4-yl)amino)-3-(perfluoroethyl)-2H-indazol-2-yl)-1,2,4-oxadiazol-5-yl)methyl)-1H-pyrazole-4-carboxamide;
1-(tert-butyl)-N-((3-(7-(((3S,4R)-3-fluoro-1-methylpiperidin-4-yl)amino)-3-(perfluoroethyl)-2H-indazol-2-yl)-1,2,4-oxadiazol-5-yl)methyl)-1H-pyrrole-3-carboxamide;
N-((5-(7-(((3S,4R)-3-fluoro-1-methylpiperidin-4-yl)amino)-3-(perfluoroethyl)pyrazolo[1,5-a]pyridin-2-yl)-1,3,4-thiadiazol-2-yl)methyl)cyclopropanecarboxamide;
1-(tert-butyl)-N-((5-(7-(((3S,4R)-3-fluoro-1-methylpiperidin-4-yl)amino)-3-(perfluoroethyl)pyrazolo[1,5-a]pyridin-2-yl)-1,3,4-thiadiazol-2-yl)methyl)-1H-pyrazole-4-carboxamide; and
1-(tert-butyl)-N-((5-(7-(((3S,4R)-3-fluoro-1-methylpiperidin-4-yl)amino)-3-(perfluoroethyl)pyrazolo[1,5-a]pyridin-2-yl)-1,3,4-thiadiazol-2-yl)methyl)-1H-pyrrole-3-carboxamide.

In some embodiments, the compound is of the formula;

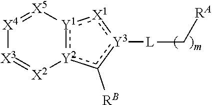

wherein;
X¹ is CR¹ or N;
R¹ is hydrogen, halogen, cyano, —OR⁴, —NR⁴R⁵, —C(═O)R⁴, —OC(═O)R⁴, —C(═O)OR⁴, —C(═O)NR⁴R5, —SR⁴, —S(═O)R⁴, —S(O)₂R4, —NR⁴C(═O)R⁵, —R⁴C(═O)R⁵, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
each of X², X³, X⁴, and X⁵ are CH, N, CR² or CR³, wherein two or more of X², X³, X⁴, and X⁵ are independently CH, CR², or CR³;
each of Y¹, Y², and Y³ are C or N, wherein one of Y¹, Y², and Y³ is N;
R^{A} is hydrogen, —OR⁶, —NR⁶R⁷, —C(═O)R⁶, —R⁶C(═O)R⁷, —OC(═O)R⁶, —OC(═O)NR⁶R⁷, —C(═O)OR⁶, —NR⁶C(═O)OR⁷, —C(═O)NR⁶R⁷, —SR⁶, —S(═O)R⁶, —S(O)₂R⁶, —S(O)₂NR⁶R⁷, —NR⁶S(O)₂R⁷, —NR⁶C(═O)R⁷, —NR⁶C(═O)NR⁷R⁸, —SiR⁶R⁷R⁸, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl. or optionally substituted 5-10 membered heteroaryl;
R^{B} is halogen, cyano, hydroxyl, —NR⁸R⁹, —OR⁸, —C(═O)NR⁸R⁹, —C(═O)R⁸, —C(═O)OR⁸, —NR⁸C(═O)OR⁹, —OC(═O)R⁸, —OC(═O)NR⁸R⁹, —C(═O)NR⁸R⁹, —NR⁸C(═O)R⁹, —NR⁷C(═O)NR⁸R⁹, —SR⁸, —S(═O)R⁸, —S(O)₂R⁸, —S(O)₂NR⁸R⁹, —NR⁸S(O)₂R⁹, —R⁸C(═O)R⁹, —NR⁸C(═O)R⁹, —NR⁷C(═O)NR⁸R⁹, optionally substituted C1-C6 alkyl, C1-C6 haloalkyl, optionally substituted C2-C6 alkenyl, optionally substituted C2-C6 alkynyl, optionally substituted C3-C10 cycloalkyl, optionally substituted phenyl, optionally substituted 3-12 membered heterocyclyl, or optionally substituted 5-10 membered heteroaryl;
each R² is

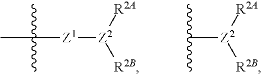

Z¹ is —C(═O)—, —S(O)₂-optionally substituted C1-C6 alkylene, optionally substituted C2-C6 alkenylene, optionally substituted C2-C6 alkynylene, or an optionally substituted C3-C4 cycloalkylene;
Z² is CR^{2C}, N, or O; wherein when Z² is O, R^{2B} is absent;
R^{2A} and R^{2B} are independently hydrogen, —C(═O)R¹⁰, —C(═O)OR¹⁰, —C(═O)NR¹⁰R¹¹, —S(═O)R¹⁰, —S(O)₂R¹⁰, optionally substituted C1-C6 alkyl, optionally substituted C2-C6 alkenyl, optionally substituted C2-C6 alkynyl, optionally substituted C3-C10 cycloalkyl. optionally substituted phenyl, optionally substituted 4-12 membered heterocyclyl, optionally substituted 5-10 membered heteroaryl; or R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl; or Z² is O and R^{B} is absent;
R^{2C} is hydrogen. halogen, or C1-C6 alkyl;
each R³ is independently halogen, cyano, —NR¹²R¹³, —OR¹², —C(═O)NR¹²R¹³, —C(═O)R¹², —C(═O)OR¹², —OC(═O)R¹², —NR¹²(C═O)NR¹³R¹⁴, —SR¹², —S(═O)R¹², —S(O)₂R¹², —S(O)₂NR¹²R¹³, —NR¹²S(O)₂NR¹³R¹⁴, —R¹²C(═O)R¹³, —NR¹²C(═O)R¹³, optionally substituted C1-C6 alkyl, optionally substituted C2-C6 alkenyl, optionally substituted C2-C6 alkynyl, optionally substituted C3-C6 cycloalkyl, optionally substituted phenyl, optionally substituted 4-6 membered heterocyclyl. or optionally substituted 5-6 membered heteroaryl;
   L is an optionally substituted C₂-C₆ alkynylene;
m is 0.1, or 2; and
each R⁴, R⁵, R⁶, R⁷, R⁸, R⁹, R¹⁰, R¹¹, R¹², R¹³, and R¹⁴ are independently hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, the compound is;
(3S,4R)-3-fluoro-N-(2-{3-[(4-methanesulfonyl-2-methoxyphenyl)amino]prop-1-yn-1-yl}-3-(2,2,2-trifluoroethyl)imidazo[1,2-a]pyridin-8-yl)-1-methylpiperidin-4-amine
N-(2-{3-[(4-methanesulfonyl-2-methoxyphenyl)amino]prop-1-yn-1-yl}-3-(2,2,2-trifluoroethyl)imidazo[1,2-a]pyridin-8-yl)-1-methylpiperidin-4-amine
2-{3-[(4-methanesulfonyl-2-methoxyphenyl)amino]prop-1-yn-1-yl}-N-(oxan-4-yl)-3-(2,2,2-trifluoroethyl)imidazo[1,2-a]pyridin-8-amine
N-(2-{3-[(4-methanesulfonyl-2-methoxyphenyl)amino]prop-1-yn-1-yl}-3-(2,2,2-trifluoroethyl)imidazo[1,2-a]pyridin-8-yl)-1-(2-methoxyethyl)piperidin-4-amine
4-[(2-{3-[(4-methanesulfonyl-2-methoxyphenyl)amino]prop-1-yn-1-yl)-3-(2,2,2-trifluoroethyl)imidazo[1,2-a]pyridin-8-yl)amino]-1lambda6-thiane-1,1-dione
N-(2-{3-[(4-methanesulfonyl-2-methoxyphenyl)amino]prop-1-yn-1-yl}-3-(2,2,2-trifluoroethyl) imidazo[1,2-a]pyridin-8-yl)-1-(2-methanesulfonylethyl) piperidin-4-amine
N-(2-{3-[(4-methanesulfonyl-2-methoxyphenyl)amino]prop-1-yn-1-yl}-3-(2,2,2-trifluoroethyl)imidazo[1,2-a]pyridin-8-yl)-2-methyl-2-azaspiro[3.3]heptan-6-amine
2-{3-[(4-methanesulfonyl-2-methoxyphenyl)amino]prop-1-yn-1-yl}-N-{2-oxaspiro[3.3]heptan-6-yl}-3-(2,2,2-trifluoroethyl)imidazo[1,2-a]pyridin-8-amine
(3R,4S)-3-fluoro-N-(2-{3-[(4-methanesulfonyl-2-methoxyphenyl)amino]prop-1-yn-1-yl}-3-(2,2,2-trifluoroethyl) imidazo[1,2-a]pyridin-8-yl)-1-methylpiperidin-4-amine methyl 4-[(2-{3-[(4-methanesulfonyl-2-methoxyphenyl)amino]prop-1-yn-1-yl}-3-(2,2,2-trifluoroethyl)imidazo[1,2-a]pyridin-8-yl)amino]piperidine-1-carboxylate
1-cyclopropyl-N-(2-{3-[(4-methanesulfonyl-2-methoxyphenyl)amino]prop-1-yn-1-yl}-3-(2,2,2-trifluoroethyl)imidazo[1,2-a]pyridin-8-yl)piperidin-4-amine
(3S,4R)-3-fluoro-N-(2-{3-[(4-methanesulfonyl-2-methoxyphenyl)amino]prop-1-yn-1-yl}-3-(2,2,2-trifluoroethyl)imidazol 1,2-a]pyridin-8-yl)piperidin-4-amine
4-[(2-{3-[(4-methanesulfonyl-2-methoxyphenyl)amino]prop-1-yn-1-yl)-3-(2,2,2-trifluoroethyl)imidazo[1,2-a]pyridin-8-yl)amino]-N-methylpiperidine-1-carboxamide
1-isopropyl-N-(2-{3-[(4-methanesulfonyl-2-methoxyphenyl)amino]prop-1-yn-1-yl}-3-(2,2,2-trifluoroethyl)imidazo[1,2-a]pyridin-8-yl)piperidin-4-amine
1-{4-[(2-{3-[(4-methanesulfonyl-2-methoxyphenyl)amino]prop-1-yn-1-yl}-3-(2,2,2-trifluoroethyl)imidazo[1,2-a]pyridin-8-yl)amino]piperidin-1-yl}ethanone
1-{4-[(2-{3-1(4-methanesulfonyl-2-methoxyphenyl)amino]prop-1-yn-1-yl}-3-(2,2,2-trifluoroethyl)imidazo[1,2-a]pyridin-8-yl)amino]piperidin-1-yl}-2-methylpropan-2-ol
(2S)-1-{4-[(2-{3-[(4-methanesulfonyl-2-methoxyphenyl)amino]prop-1-yn-1-yl}-3-(2,2,2-trifluoroethyl)imidazo[1,2-a]pyridin-8-yl)amino]piperidin-1-yl}-3-methoxypropan-2-ol
(2R)-1-{4-1(2-{3-[(4-methanesulfonyl-2-methoxyphenyl)amino]prop-1-yn-1-yl)-3-(2,2,2-trifluoroethyl)imidazo[1,2-a]pyridin-8-yl)amino]piperidin-1-yl -3-methoxypropan-2-ol
(3S,4R)—N-(3-ethyl-2-{3-[(4-methanesulfonyl-2-methoxyphenyl)amino]prop-1-yn-1-yl}imidazo[1,2-a]pyridin-8-yl)-3-fluoro-1-methylpiperidin-4-amine
N-(3-ethyl-2-{3-[(4-methanesulfonyl-2-methoxyphenyl)amino]prop-1-yn-1-yl)imidazo[1,2-a]pyridin-8-yl)-1-methylpiperidin-4-amine
4-{[3-(3-ethoxy-8-{[(3S,4R)-3-fluoro-1-methylpiperidin-4-yl]amino}imidazo[1,2-a]pyridin-2-yl)prop-2-yn-1-yl]amino}-3-methoxy-N-methylbenzamide
(3S,4R)-3-fluoro-1-methyl-N-[2-(prop-1-yn-1-yl)-3-(2,2,2-trifluoroethyl)imidazol 1,2-a]pyridin-8-yl]piperidin-4-amine
1-methyl-N-[2-(prop-1-yn-1-yl)-3-(2,2,2-trifluoroethyl)imidazo[1,2-a]pyridin-8-yl]piperidin-4-amine
(3S,4R)-3-fluoro-1-methyl-N-[2-(prop-1-yn-1-yl)-3-(2,2,2-trifluoroethyl)pyrazolo[1,5-a]pyridin-7-yl]piperidin-4-amine
N-[3-ethenyl-2-(prop-1-yn-1-yl)imidazo[1,2-a]pyridin-8-yl]-1-methylpiperidin-4-amine
(3S,4R)—N-[3-ethenyl-2-(prop-1-yn-1-yl)imidazo[1,2-a]pyridin-8-yl]-3-fluoro-1-methylpiperidin-4-amine
4-{[3-(8-{[(3S,4R)-3-fluoro-1-methylpiperidin-4-yl]amino}-3-(prop-2-enamido)imidazo[1,2-a]pyridin-2-yl)prop-2-yn-1-yl]amino}-3-methoxy-N-methylbenzamide
4-{[3-(8-{[(3S,4R)-3-fluoro-1-methylpiperidin-4-yl]amino}-3-(2-methylprop-2-enamido) imidazo[1,2-a]pyridin-2-yl)prop-2-yn-1-yl]amino}-3-methoxy-N-methylbenzamide
4-{[3-(8-{[(3S,4R)-3-fluoro-1-methylpiperidin-4-yl]amino}-3-(methylamino) imidazo[1,2-a]pyridin-2-yl) prop-2-yn-1-yl]amino}-3-methoxy-N-methylbenzamide
4-{[3-(8-{[(3S,4R)-3-fluoro-1-methylpiperidin-4-yl]amino}-3-(N-methylprop-2-enamido)imidazo[1,2-a]pyridine-2-yl)prop-2-yn-1-yl]amino}-3-methoxy-N-methylbenzamide
2-{3-[(4-methanesulfonyl-2-methoxyphenyl)amino]prop-1-yn-1-yl}-8-[(1-methylpiperidin-4-yl)aminol-3-(2,2,2-trifluoroethyl)imidazo[1,2-a]pyridin-6-ol
4-{[3-(8-[(3S,4R)-3-fluoro-1-methylpiperidin-4-yl]amino}-3-[(trifluoromethyl)sulfanyl]imidazo[1,2-a]pyridin-2-yl)prop-2-yn-1-yl]amino)-3-methoxy-N-methylbenzamide
4-{[3-(8-{[(3S,4R)-3-fluoro-1-methylpiperidin-4-yl]amino}-3-(1,1,2,2,2-pentafluoroethyl)imidazo[1,2-a]pyridin-2-yl)prop-2-yn-1-yl]amino}-3-methoxy-N-methylbenzamide
tert-butyl N-(2-{3-[(4-methanesulfonyl-2-methoxyphenyl)amino]prop-1-yn-1-yl}-8-[(1-methylpiperidin-4-yl)amino]-3-(2,2,2-trifluoroethyl) imidazo[1,2-al pyridin-6-yl) carbamate
2-{3-[(4-methanesulfonyl-2-methoxyphenyl)amino]prop-1-yn-1-yl}-N8-(1-methylpiperidin-4-yl)-3-(2,2,2-trifluoroethyl) imidazo[1,2-a]pyridine-6,8-diamine
2-{3-[(4-methanesulfonyl-2-methoxyphenyl)amino]prop-1-yn-1-yl}-N6-methyl-N8-(1-methylpiperidin-4-yl)-3-(2,2,2-trifluoroethyl) imidazol 1,2-a]pyridine-6,8-diamine
N-(2-{3-[(4-methanesulfonyl-2-methoxyphenyl)amino]prop-1-yn-1-yl}-6-phenyl-3-(2,2,2-trifluoroethyl)imidazo[1,2-a]pyridin-8-yl)-1-methylpiperidin-4-amine
4-[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino}-N-methylbenzamide
4-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino)-N-methylbenzamide
4-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino)₃-methoxy-N-methylbenzamide
4-[3-(8-{[(4R)-3,3-difluoro-1-methylpiperidin-4-yl]amino)-3-(2,2,2-trifluoroethyl)imidazo[1,2-a]pyridin-2-yl)prop-2-yn-1-yl]amino}-3-methoxy-N-methylbenzamide
4-{[3-(8-{[(4S)-3,3-difluoro-1-methylpiperidin-4-yl]amino}-3-(2,2,2-trifluoroethyl)imidazo[1,2-a]pyridin-2-yl)prop-2-yn-1-yl]amino)-3-methoxy-N-methylbenzamide tert-butyl N-[2-(3-{[2-methoxy-4-(methylcarbamoyl)phenyl]amino) prop-1-yn-1-yl)-8-[(1-methylpiperidin-4-yl)amino]-3-(2,2,2-trifluoroethyl) imidazo[1,2-a]pyridin-6-yl]carbamate
4-[(3-{6-amino-8-[(1-methylpiperidin-4-yl)aminol-3-(2,2,2-trifluoroethyl) imidazo[1,2-a]pyridin-2-yl) prop-2-yn-1-yl)amino]-3-methoxy-N-methylbenzamide
4-[(3-{6-acetamido-8-[(1-methylpiperidin-4-yl)amino]-3-(2,2,2-trifluoroethyl)imidazo[1,2-a]pyridin-2-yl}prop-2-yn-1-yl)aminol-3-methoxy-N-methylbenzamide
3-methoxy-N-methyl-4-[(3-{8-1(1-methylpiperidin-4-yl)aminol-6-(prop-2-enamido)-3-(2,2,2-trifluoroethyl)imidazo[1,2-a]pyridin-2-yl}prop-2-yn-1-yl)amino]benzamide
3-methoxy-N-methyl-4-[(3-{8-[(1-methylpiperidin-4-yl)amino]-6-(2-methylprop-2-enamido)-3-(2,2,2-trifluoroethyl)imidazol 1,2-a]pyridin-2-yl}prop-2-yn-1-yl)amino]benzamide
4-((3-[6-(2-chloro-2-fluoroacetamido)-8-[(1-methylpiperidin-4-yl)amino]-3-(2,2,2-trifluoroethyl)imidazo[1,2-a]pyridin-2-yl]prop-2-yn-1-ylamino)-3-methoxy-N-methylbenzamide
3-methoxy-N-methyl-4-[(3-{6-[(methylcarbamoyl)amino]-8-[(1-methylpiperidin-4-yl)amino]-3-(2,2,2-trifluoroethyl) imidazo[1,2-a]pyridin-2-yl}prop-2-yn-1-yl)amino]benzamide methyl N-[2-(3-{[2-methoxy-4-(methylcarbamoyl)phenyl]amino}prop-1-yn-1-yl)-8-[(I-methylpiperidin-4-yl)amino]-3-(2,2,2-trifluoroethyl) imidazo[1,2-a]pyridin-6-yl]carbamate
4-{[3-(8-{[(3S,4R)-3-fluoro-1-methylpiperidin-4-yl]aminol-3-(2,2,2-trifluoroethyl)imidazo[1,2-a]pyridin-2-yl)prop-2-yn-1-yl]amino)-3-methoxy-N,N-dimethylbenzamide
N-ethyl-4-{[3-(8-{[(3S,4R)-3-fluoro-1-methylpiperidin-4-yl]amino -3-(2,2,2-trifluoroethyl)imidazo[1,2-a]pyridin-2-yl)prop-2-yn-1-yl]amino)-3-methoxybenzamide
4-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino)-N-isopropyl-3-methoxybenzamide
N-cyclopropyl-4-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-3-methoxybenzamide
(3S,4R)-3-fluoro-N-[2-(3-{[2-methoxy-4-(pyrrolidine-1-carbonyl)phenyl]amino)prop-1-yn-1-yl)-3-(2,2,2-trifluoroethyl)imidazo[1,2-a]pyridin-8-yl]-1-methylpiperidin-4-amine
4-{[3-(8-{[(3S,4R)-3-fluoro-1-methylpiperidin-4-yl]amino}-3-(1,1,2,2,2-pentafluoroethyl)imidazo[1,2-a]pyridin-2-yl)prop-2-yn-1-yl]amino}-3-methoxy-N-methylbenzamide
5-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino}-N-methylpyridine-2-carboxamide
4-{[3-(8-{[(3S,4R)-3-fluoro-1-methylpiperidin-4-yl]amino)-3-(2,2,2-trifluoroethyl) imidazo[1,2-a]pyridin-2-yl) prop-2-yn-1-yl]amino)-N-methyl-3-(trifluoromethoxy)benzamide
4-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino}-N-methyl-3-(trifluoromethyl)benzamide
3-chloro-4-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino)-N-methylbenzamide
3-cyano-4-[3-(8-{[(3S,4R)-3-fluoro-1-methylpiperidin-4-yl]aminol-3-(2,2,2-trifluoroethyl)imidazo[1,2-a]pyridin-2-yl)prop-2-yn-1-yl]amino)-N-methylbenzamide
3-cyclopropyl-4-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino)-N-methylbenzamide
3-ethyl-4-{[3-(8-{[(3S,4R)-3-fluoro-1-methylpiperidin-4-yl]amino}-3-(2,2,2-trifluoroethyl)imidazo[1,2-a]pyridin-2-yl) prop-2-yn-1-yl]amino)-N-methylbenzamide
3-ethoxy-4-{[3-(8-{1(3S,4R)-3-fluoro-1-methylpiperidin-4-yl]amino)-3-(2,2,2-trifluoroethyl) imidazo[1,2-a]pyridin-2-yl) prop-2-yn-1-yl]amino)-N-methylbenzamide
3-(difluoromethoxy)-4-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino}-N-methylbenzamide
4-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-N-methyl-3-(2,2,2-trifluoroethoxy)benzamide
4-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-3-(2-methoxyethoxy)-N-methylbenzamide
4-{[3-(8-1[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-3-methoxybenzenesulfonamide
4-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-3-methoxy-N-methylbenzenesulfonamide
4-{[3-(8-{[(3S,4R)-3-fluoro-1-methylpiperidin-4-yl]amino}-3-(2,2,2-trifluoroethyl) imidazo[1,2-a]pyridin-2-yl)prop-2-yn-1-yl]amino}-3-methoxy-N,N-dimethylbenzenesulfonamide
(3S,4R)—N-[2-(3-{[4-(ethanesulfonyl)-2-methoxyphenyl]amino) prop-1-yn-1-yl)-3-(2,2,2-trifluoroethyl)imidazo[1,2-a]pyridin-8-yl]-3-fluoro-1-methylpiperidin-4-amine
(3S,4R)—N-[2-(3-{[4-(cyclopropanesulfonyl)-2-methoxyphenyl]amino]prop-1-yn-1-yl)-3-(2,2,2-trifluoroethyl)imidazo[1,2-a]pyridin-8-yl]-3-fluoro-1-methylpiperidin-4-amine
(3S,4R)-3-fluoro-N-(2-(3-[(2-fluoro-4-methanesulfonylphenyl)amino]prop-1-yn-1-yl)-3-(2,2,2-trifluoroethyl)imidazo[1,2-a]pyridin-8-yl)-1-methylpiperidin-4-amine
4-(8-{[(3S,4R)-3-fluoro-1-methylpiperidin-4-yl]amino}-3-(2,2,2-trifluoroethyl)imidazo[1,2-a]pyridin-2-yl)-2-methylbut-3-yn-2-ol
(2R)-4-(8-{[(3S,4R)-3-fluoro-1-methylpiperidin-4-yl]amino)-3-(2,2,2-trifluoroethyl)imidazo[1,2-a]pyridin-2-yl)but-3-yn-2-ol
(2S)-4-(8-{[(3S,4R)-3-fluoro-1-methylpiperidin-4-yl]amino}-3-(2,2,2-trifluoroethyl)imidazo[1,2-a]pyridin-2-yl)but-3-yn-2-ol
(1S)-3-(8-{[(3S,4R)-3-fluoro-1-methylpiperidin-4-yl]amino)-3-(2,2,2-trifluoroethyl)imidazo[1,2-a]pyridin-2-yl)-1-phenylprop-2-yn-1-ol
(1R)-3-(8-{1[(3S,4R)-3-fluoro-1-methylpiperidin-4-yl]amino}-3-(2,2,2-trifluoroethyl)imidazo[1,2-a]pyridin-2-yl)-1-phenylprop-2-yn-1-ol
(3S,4R)—N-[2-(4-aminobut-1-yn-1-v)-3-(2,2,2-trifluoroethyl)imidazo[1,2-a]pyridin-8-yl]-3-fluoro-1-methylpiperidin-4-amine
(3S,4R)—N-[2-(3-aminoprop-1-yn-1-yl)-3-(2,2,2-trifluoroethyl)imidazo[1,2-a]pyridin-8-yl]-3-fluoro-1-methylpiperidin-4-amine
(3S,4R)-3-fluoro-N-(2-{3-[(4-methanesulfonylphenyl)amino]prop-1-yn-1-yl}-3-(2,2,2-trifluoroethyl)imidazo[1,2-a]pyridin-8-yl)-1-methylpiperidin-4-amine
(3S,4R)-3-fluoro-N-[2-(3-{[2-methoxy-4-(propane-2-sulfonyl)phenyl]amino}prop-1-yn-1-yl)-3-(2,2,2-trifluoroethyl) imidazo[1,2-a]pyridin-8-yl]-1-methylpiperidin-4-amine
(3S,4R)-3-fluoro-N-[2-(3-{2-methoxy-4-(propane-2-sulfonyl)phenyl]amino}prop-1-yn-1-yl)-3-[(trifluoromethyl)sulfanyl]imidazo[1,2-a]pyridin-8-yl]-1-methylpiperidin-4-amine
(3S,4R)—N-[2-(3-{[4-(ethanesulfonyl)-2-methoxyphenyl]amino}prop-1-yn-1-yl)-3-[(trifluoromethyl)sulfanyl]imidazo[1,2-a]pyridin-8-yl]-3-fluoro-1-methylpiperidin-4-amine
(3S,4R)—N-[2-(3-{[4-(ethanesulfonyl)-2-methoxyphenyl]amino}prop-1-yn-1-yl)-3-[(trifluoromethyl)sulfanyl]imidazol 1,2-a]pyridin-8-yl]-3-fluoropiperidin-4-amine
(3S,4R)—N-[2-(3-{[4-(cyclopropanesulfonyl)-2-methoxyphenyl]amino) prop-1-yn-1-yl)-3-[(trifluoromethyl)sulfanyl]imidazo[1,2-a]pyridin-8-yl]-3-fluoro-1-methylpiperidin-4-amine
(3S,4R)-3-fluoro-N-(2-{3-[(4-methanesulfonyl-2-methoxyphenyl)amino]prop-1-yn-1-yl}-3-[(trifluoromethyl)sulfanyl]imidazo[1,2-a]pyridin-8-yl)-1-methylpiperidin-4-amine
(3S,4R)-3-fluoro-N-(2-{3-[(4-methanesulfonyl-2-methoxyphenyl)amino]prop-1-yn-1-yl)-3-[(trifluoromethyl)sulfanyl]imidazo[1,2-a]pyridin-8-yl)piperidin-4-amine)
(3S,4R)-3-fluoro-N-(2-(3-[(5-methanesulfonyl-2-methoxyphenyl)amino]prop-1-yn-1-yl}-3-[(trifluoromethyl)sulfanyl]imidazo[1,2-a]pyridin-8-yl)-1-methylpiperidin-4-amine
N-ethyl-4-{[3-(8-{[(3S,4R)-3-fluoro-1-methylpiperidin-4-yl]amino}-3-[(trifluoromethyl)sulfanyl]imidazo[1,2-a]pyridin-2-yl)prop-2-yn-1-yl]amino}-3-methoxybenzamide
N-cyclopropyl-4-{[3-(8-{[(3S,4R)-3-fluoro-1-methylpiperidin-4-yl]amino)-3-[(trifluoromethyl) sulfanyl]imidazo[1,2-a]pyridin-2-yl)prop-2-yn-1-yl]amino}-3-methoxybenzamide
(3S,4R)—N-[2-(3-{[4-(azetidine-1-carbonyl)-2-methoxyphenyl]amino)prop-1-yn-1-yl)-3-[(trifluoromethyl)sulfanyl]imidazol 1,2-a]pyridin-8-yl]-3-fluoro-1-methylpiperidin-4-amine
4-{[3-(8-{[(3S,4R)-3-fluoro-1-methylpiperidin-4-yl]amino}-3-[(trifluoromethyl)sulfanyl]imidazo[1,2-a]pyridin-2-yl) prop-2-yn-1-yl]amino)-3-methoxy-N, N-dimethylbenzamide
4-[3-(8-{[(3S,4R)-3-fluoro-1-methylpiperidin-4-yl]amino}-3-1(trifluoromethyl)sulfanyl]imidazo[1,2-a]pyridin-2-yl)prop-2-yn-1-yl]amino}-N-isopropyl-3-methoxybenzamide
4-({3-[6-(2-fluoroprop-2-enamido)-8-[(1-methylpiperidin-4-yl)amino]-3-(2,2,2-trifluoroethyl)imidazo[1,2-a]pyridin-2-yl]prop-2-yn-1-yl}amino)-3-methoxy-N-methylbenzamide
4-((3-[6-(2-chloroacetamido)-8-[(1-methylpiperidin-4-yl)amino]-3-(2,2,2-trifluoroethyl)imidazo[1,2-a]pyridin-2-yl]prop-2-yn-1-ylamino)-3-methoxy-N-methylbenzamide
5-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino-6-methoxy-N-methylpyridine-2-carboxamide
5-{[3-(8-{[(3S,4R)-3-fluoro-1-methylpiperidin-4-yl]amino}-3-[(trifluoromethyl)sulfanyl]imidazo[1,2-a]pyridin-2-yl)prop-2-yn-1-yl]amino}-6-methoxy-N-methylpyridine-2-carboxamide
5-{[3-(8-{[(3S,4R)-3-fluoro-1-methylpiperidin-4-yl]amino}-3-[(trifluoromethyl)sulfanyl]imidazol 1,2-a]pyridin-2-yl)prop-2-yn-1-yl]amino}-N-methylpyridine-2-carboxamide
5-{[3-(8-{[(3S,4R)-3-fluoro-1-methylpiperidin-4-yl]amino)-3-(2,2,2-trifluoroethyl)imidazo[1,2-a]pyridin-2-yl)prop-2-yn-1-yl]amino}-6-methoxy-2,3-dihydroisoindol-1-one
5-{[3-(8-{[(3S,4R)-3-fluoro-1-methylpiperidin-4-yl]amino}-3-[(trifluoromethyl)sulfanyl]imidazo[1,2-a]pyridin-2-yl)prop-2-yn-1-yl]amino}-6-methoxy-2,3-dihydroisoindol-1-one
4-{[3-(8-{[(3S,4R)-3-fluoro-1-methylpiperidin-4-yl]amino)-3-(trifluoromethyl)imidazo 1,2-a]pyridin-2-yl)prop-2-yn-1-yl]amino}-3-methoxy-N-methylbenzamide
4-{[3-(7-fluoro-8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-3-methoxy-N-methylbenzamide
4-{[3-(6-fluoro-8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino}-3-methoxy-N-methylbenzamide
4-{[3-(8-{[(3S,4R)-3-fluoropiperidin-4-yl]amino}-3-[(trifluoromethyl)sulfanyl]imidazo[1,2-a]pyridin-2-yl)prop-2-yn-1-yl]amino}-3-methoxy-N-methylbenzamide
4-{[3-(8-{[(3R,4R)-3-fluorooxan-4-yl]amino}-3-[(trifluoromethyl)sulfanyl]imidazo[1,2-a]pyridin-2-yl)prop-2-yn-1-yl]amino) -3-methoxy-N-methylbenzamide
4-{[3-(8-1[(3R,4S)-4-fluoropyrrolidin-3-yl]amino}-3-[(trifluoromethyl)sulfanyl]imidazo[1,2-a]pyridin-2-yl) prop-2-yn-1-yl]amino}-3-methoxy-N-methylbenzamide
4-[3-(8-1[(3R,4S)-4-fluoro-1-methylpyrrolidin-3-yl]amino -3-[(trifluoromethyl)sulfanyl]imidazo[1,2-a]pyridin-2-yl)prop-2-yn-1-yl]amino}-3-methoxy-N-methylbenzamide
4-{[3-(8-{[(3S,4R)-3-fluoro-1-methylpiperidin-4-yl]amino}-3-[(trifluoromethyl)sulfanyl]imidazol 1,2-a]pyridin-2-yl)prop-2-yn-1-yl]amino}-3-(2-methoxyethoxy)-N-methylbenzamide
3-(difluoromethoxy)-4-{[3-(8-{1[(3S,4R)-3-fluoro-1-methylpiperidin-4-yl]amino)-3-[(trifluoromethyl)sulfanyl]imidazo[1,2-a]pyridin-2-yl)prop-2-yn-1-yl]amino}-N-methylbenzamide
4-[(3-{3-[(difluoromethyl)sulfanyl]-8-{1[(3S,4R)-3-fluoro-1-methylpiperidin-4-yl]amino}imidazo[1,2-a]pyridin-2-yl}prop-2-yn-1-yl)amino]-3-methoxy-N-methylbenzamide
3-{[3-(8-{[(3S,4R)-3-fluoro-1-methylpiperidin-4-yl]amino}-3-[(trifluoromethyl)sulfanyl]imidazo[1,2-a]pyridin-2-yl) prop-2-yn-1-yl]amino}-4-methoxy-N-methylbenzamide
N-ethyl-3-{[3-(8-{[(3S,4R)-3-fluoro-1-methylpiperidin-4-yl]aminol-3-[(trifluoromethyl)sulfanyl]imidazo[1,2-a]pyridin-2-yl)prop-2-yn-1-yl]amino-4-methoxybenzamide
N-cyclopropyl-3-{[3-(8-{1[(3S,4R)-3-fluoro-1-methylpiperidin-4-yl]amino}-3-[(trifluoromethyl) sulfanyl]imidazo[1,2-a]pyridin-2-yl)prop-2-yn-1-yl]amino}-4-methoxybenzamide
6-{[3-(8-{[(3S,4R)-3-fluoro-1-methylpiperidin-4-yl]amino}-3-(2,2,2-trifluoroethyl)imidazo[1,2-a]pyridin-2-yl)prop-2-yn-1-yl]amino)-7-methoxy-3,4-dihydro-2H-isoquinolin-1-one
2-chloro-4-{[3-(8-{[(3S,4R)-3-fluoro-1-methylpiperidin-4-yl]amino}-3-(2,2,2-trifluoroethyl) imidazol 1,2-a]pyridin-2-yl)prop-2-yn-1-yl]amino}-5-methoxy-N-methylbenzamide
(3S,4R)-3-fluoro-1-methyl-N-[2-[3-(phenylamino)prop-1-yn-1-yl]-3-(2,2,2-trifluoroethyl)imidazo[1,2-a]pyridin-8-yl}piperidin-4-amine
3-{[3-(8-1[(3S,4R)-3-fluoro-1-methylpiperidin-4-yl]amino}-3-[(trifluoromethyl)sulfanyl]indolizin-2-yl)prop-2-yn-1-yl]amino}-4-methoxy-N-methylbenzamide
3-{[3-(8-{[(3S,4R)-3-fluoropiperidin-4-yl]amino}-3-[(trifluoromethyl)sulfanyl]indolizin-2-yl)prop-2-yn-1-yl]amino}-4-methoxy-N-methylbenzamide
4-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-3-methoxy-N-methylbenzamide
3-{[3-(8-{[(3S,4R)-3-fluoro-1-methylpiperidin-4-yl]amino)-3-[(trifluoromethyl)sulfanyl]indolizin-2-yl)prop-2-yn-1-yl]amino}-4-methoxy-N-methylbenzenesulfonamide
4-[(3-{3-[(difluoromethyl)sulfanyl]-8-{1(3S,4R)-3-fluoro-1-methylpiperidin-4-yl]amino)indolizin-2-yl)prop-2-yn-1-yl)amino]-3-methoxy-N-methylbenzamide
4-{[3-(5-{[(3S,4R)-3-fluoro-1-methylpiperidin-4-yl]amino}-1-|(trifluoromethyl)sulfanyl]indolizin-2-yl)prop-2-yn-1-yl]amino}-3-methoxy-N-methylbenzamide
3-{[3-(6-fluoro-8-{1[(3S,4R)-3-fluoro-1-methylpiperidin-4-yl]amino)-3-[(trifluoromethyl)sulfanyl]imidazo[1,2-a]pyridin-2-yl)prop-2-yn-1-yl]amino)-4-methoxy-N-methylbenzamide
4-{[3-(1-{[(3S,4R)-3-fluoro-1-methylpiperidin-4-yl]amino)-6-[(trifluoromethyl)sulfanyl]pyrrolo[1,2-a]pyrazin-7-yl) prop-2-yn-1-yl]amino)-3-methoxy-N-methylbenzamide
3-{[3-(1-{[(3S,4R)-3-fluoro-1-methylpiperidin-4-yl]amino}-6-[(trifluoromethyl)sulfanyl]pyrrolo[1,2-a]pyrazin-7-yl)prop-2-yn-1-yl]amino)-4-methoxy-N-methylbenzamide
(3S,4R)—N-[7-(3-{[4-(dimethylphosphoryl)-2-methoxyphenyl]amino)prop-1-yn-1-yl)-6-[(trifluoromethyl)sulfanyl]pyrrolo[1,2-a]pyrazin-1-yl]-3-fluoro-1-methylpiperidin-4-amine
8-{[3-(1-1[(3S,4R)-3-fluoro-1-methylpiperidin-4-yl]amino}-6-[(trifluoromethyl)sulfanyl]pyrrolo[1,2-a]pyrazin-7-yl)prop-2-yn-1-yl]amino)-N-methylimidazo[1,2-a]pyridine-6-carboxamide
4-[(3-{6-[(difluoromethyl)sulfanyl]-1-{[(3S,4R)-3-fluoro-1-methylpiperidin-4-yl]amino}pyrrolo[1,2-a]pyrazin-7-yl}prop-2-yn-1-yl)aminol-3-methoxy-N-methylbenzamide
4-[3-(8-1[(3S,4R)-3-fluoro-1-methylpiperidin-4-yl]amino)-3-[(trifluoromethyl)sulfanyl]imidazo[1,2-a]pyrazin-2-yl)prop-2-yn-1-yl]amino)-3-methoxy-N-methylbenzamide
4-{[3-(7-1[(3S,4R)-3-fluoro-1-methylpiperidin-4-yl]amino}-3-[(trifluoromethyl)sulfanyl]pyrazolo[1,5-c]pyrimidin-2-yl)prop-2-yn-1-yl]amino}-3-methoxy-N-methylbenzamide
4-{[3-(7-{[(3S,4R)-3-fluoro-1-methylpiperidin-4-yl]amino}-3-[(1-fluoroethyl)sulfanyl]pyrazolo[1,5-c]pyrimidin-2-yl)prop-2-yn-1-yl]amino)-3-methoxy-N-methylbenzamide
3-{[3-(7-{[(3S,4R)-3-fluoro-1-methylpiperidin-4-yl]amino}-3-[(trifluoromethyl)sulfanyl]pyrazolo[1,5-a]pyrimidin-2-yl)prop-2-yn-1-yl]amino}-4-methoxy-N-methylbenzamide
4-[(3-{3-[(difluoromethyl)sulfanyl]-7-{[(3S,4R)-3-fluoro-1-methylpiperidin-4-yl]amino}pyrazolo[1,5-a]pyridin-2-yl prop-2-yn-1-yl)amino]-3-methoxy-N-methylbenzamide
4-{[3-(7-{[(3S,4R)-3-fluoro-1-methylpiperidin-4-yl]amino}-3-[(trifluoromethyl)sulfanyl]pyrazolo[1,5-a]pyridin-2-yl)prop-2-yn-1-yl]amino) -3-methoxy-N-methylbenzamide
rel-4-{[3-(8-{[(1R,5R,6S,7S)-6-fluoro-3-oxa-9-azabicyclo[3.3.11 nonan-7-yl]amino}-3-[(trifluoromethyl)sulfanyl]imidazo[1,2-a]pyridin-2-yl) prop-2-yn-1-yl]aminol-3-methoxy-N-methylbenzamide
4-{[3-(8-{[(3R,4R)-3-fluorooxan-4-yl]amino)-3-[(trifluoromethyl)sulfanyl]imidazo[1,2-a]pyridin-2-yl)prop-2-yn-1-yl]amino}-N-methyl-3-[(1-methylazetidin-3-yl)oxy]benzamide
N-[3-(8-{[(3S,4R)-3-fluoro-1-methylpiperidin-4-yl]amino}-3-[(trifluoromethyl) sulfanyl]imidazo[1,2-a]pyridin-2-yl) prop-2-yn-1-yl]-5-methoxy-2H-indazol-6-amine
4-{[3-(8-{[(4S)-3,3-difluoro-1-methylpiperidin-4-yl]amino}-3-(2,2,2-trifluoroethyl)imidazo[1,2-a]pyridin-2-yl)prop-2-yn-1-yl]amino)-3-methoxy-N-methylbenzamide
4-{[3-(8-1[(1S,2S,3R,5R)-2-fluoro-8-azabicyclo[3.2.1]octan-3-yl]amino)-3-[(trifluoromethyl)sulfanyl]imidazo[1,2-a]pyridin-2-yl)prop-2-yn-1-yl]amino}-3-methoxy-N-methylbenzamide
rel-4-{3-(8-{[(1R,5R,6S,7S)-6-fluoro-3-oxa-9-azabicyclo[3.3.1]nonan-7-yl]amino)-3-[(trifluoromethyl)sulfanyl]imidazo[1,2-a]pyridin-2-yl) prop-2-yn-1-yl]amino)-3-methoxy-N-methylbenzamide
4-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-[(2R)-2-hydroxypropyl]-3-methoxybenzamide
4-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-[(2S)-2-hydroxypropyl]-3-methoxybenzamide
4-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-[(1-hydroxycyclopropyl)methyl]-3-methoxybenzamide
4-[3-(8-{[(3S,4R)-3-fluoro-1-methylpiperidin-4-yl]amino -3-[(trifluoromethyl)sulfanyl]indolizin-2-yl)prop-2-yn-1-yl]amino}-N-(2-methanesulfonylethyl)benzamide
1-(4-{[3-(8-{[(3S,4R)-3-fluoropiperidin-4-yl]amino}-3-[(trifluoromethyl)sulfanyl]indolizin-2-yl)prop-2-yn-1-yl]amino}-3-methoxybenzoyl)-3-methylazetidin-3-ol
4-{[3-(8-{[(3S,4R)-3-fluoropiperidin-4-yl]amino)-3-[(trifluoromethyl)sulfanyl]indolizin-2-yl)prop-2-yn-1-yl]amino)-N-methylbenzamide
4-{[3-(8-{[(3S,4R)-3-fluoro-1-(oxetan-3-yl)piperidin-4-yl]amino}-3-[(trifluoromethyl)sulfanyl]indolizin-2-yl)prop-2-yn-1-yl]aminol-3-methoxy-N-methylbenzamide
4-{[3-(8-{[(3S,4R)-3-fluoro-1-methylpiperidin-4-yl]amino}-3-|(trifluoromethyl)sulfanyl]indolizin-2-yl)prop-2-yn-1-yl]amino}-3-(fluoromethoxy)-N-methylbenzamide
4-{[3-(8-{[(3S,4R)-3-fluoro-1-methylpiperidin-4-yl]aminol-3-[(trifluoromethyl)sulfanyl]indolizin-2-yl)prop-2-yn-1-yl]amino}-3-hydroxy-N-methylbenzamide
4-{[3-(8-{[(3S,4R)-3-fluoropiperidin-4-yl]amino}-3-[(trifluoromethyl)sulfanyl]indolizin-2-yl)prop-2-yn-1-yl]amino)-3-hydroxy-N-methylbenzamide
3-chloro-4-{[3-(8-{l(3S,4R)-3-fluoro-1-methylpiperidin-4-yl]amino}-3-[(trifluoromethyl)sulfanyl]indolizin-2-yl)prop-2-yn-1-yl]amino)-N-methylbenzamide
4-{[3-(1-{[(3S,4R)-3-fluoro-1-methylpiperidin-4-yl]amino}-6-[(trifluoromethyl)sulfanyl]pyrrolo[1,2-a]pyrazin-7-yl)prop-2-yn-1-yl]amino)-3-hydroxy-N-methylbenzamide
3-(difluoromethyl)-4-{[3-(8-[(3S,4R)-3-fluoro-1-methylpiperidin-4-yl]amino)-3-[(trifluoromethyl)sulfanyl]indolizin-2-yl)prop-2-yn-1-yl]amino}-N-methylbenzamide
3-fluoro-4-{[3-(8-{[(3S,4R)-3-fluoro-1-methylpiperidin-4-yl]amino}-3-|(trifluoromethyl)sulfanyl]indolizin-2-yl)prop-2-yn-1-yl]amino}-N-methylbenzamide
3-cyano-4-{[3-(8-{[(3S,4R)-3-fluoro-1-methylpiperidin-4-yl]amino}-3-[(trifluoromethyl) sulfanyl]indolizin-2-yl)prop-2-yn-1-yl]amino}-N-methylbenzamide
3-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N2,N6-dimethylpyridine-2,6-dicarboxamide
5-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-6-methoxy-N-methylpyridine-2-carboxamide
4-{[3-(8-{1[(3S,4R)-3-fluoro-1-methylpiperidin-4-yl]amino}-3-[(trifluoromethyl)sulfanyl]indolizin-2-yl)prop-2-yn-1-yl]amino}-N1,N3-dimethylbenzene-1,3-dicarboxamide
3-{[3-(8-{[(3S,4R)-3-fluoro-1-(oxetan-3-yl)piperidin-4-yl]amino}-3-[(trifluoromethyl)sulfanyl]indolizin-2-yl)prop-2-yn-1-yl]amino}-4-methoxy-N-methylbenzamide
3-{[3-(8-{[(3S,4R)-3-fluoropiperidin-4-yl]amino}-3-[(trifluoromethyl)sulfanyl]indolizin-2-yl)prop-2-yn-1-yl]amino-4-hydroxy-N-methylbenzamide
3-{[3-(8-{[(3S,4R)-3-fluoro-1-[(2S*)-2-hydroxypropyl]piperidin-4-yl]amino}-3-|(trifluoromethyl)sulfanyl]indolizin-2-yl)prop-2-yn-1-yl]amino}-4-methoxy-N-methylbenzamide
3-{[3-(8-{[(3S,4R)-3-fluoro-1-[(2S*)-2-hydroxypropyl]piperidin-4-yl]amino})-3-[(trifluoromethyl)sulfanyl]indolizin-2-yl)prop-2-yn-1-yl]amino}-4-methoxy-N-methylbenzamide
3-{[3-(8-{[(3S,4R)-3-fluoro-1-methylpiperidin-4-yl]amino)-3-[(trifluoromethyl)sulfanyl]indolizin-2-yl)prop-2-yn-1-yl]amino}-N-(2-hydroxy-2-methylpropyl)-4-(oxetan-3-yloxy)benzamide
3-[3-(8-{[(3S,4R)-3-fluoro-1-methylpiperidin-4-yl]amino}-3-[(trifluoromethyl)sulfanyl]indolizin-2-yl)prop-2-yn-1-yl]amino)-N-methyl-4-[(1s,3s)-3-hydroxycyclobutoxy]benzamide
3-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-methyl-4-[(1r,3r)-3-hydroxycyclobutoxy]benzamide
8-{[3-(8-[(3R,4S)-3-fluoro-1-methylpiperidin-4-yl]amino}-3-[(trifluoromethyl)sulfanyl]indolizin-2-yl)prop-2-yn-1-yl]amino}-N-methylimidazo[1,2-a]pyridine-6-carboxamide
2-(3-{[6-(dimethylphosphoryl)-4-methoxypyridin-3-yl]amino)prop-1-yn-1-yl)-N-[(3S,4R)-3-fluoro-1-methylpiperidin-4-yl]-3-[(trifluoromethyl)sulfanyl]indolizin-8-amine
2-(3-{[6-(dimethylphosphoryl)pyridin-3-yl]amino)prop-1-yn-1-yl)-N-[(3S,4R)-3-fluoro-1-methylpiperidin-4-yl]-3-[(trifluoromethyl)sulfanyl]indolizin-8-amine
2-(3-{[5-(dimethylphosphoryl)pyridin-2-yl]amino}prop-Il-yn-1-yl)-N-[(3S,4R)-3-fluoro-1-methylpiperidin-4-yl]-3-[(trifluoromethyl)sulfanyl]indolizin-8-amine
2-(3-{[5-(dimethylphosphoryl)-3-methoxypyridin-2-yl]amino}prop-1-yn-1-yl)-N-[(3S,4R)-3-fluoro-1-methylpiperidin-4-yl]-3-[(trifluoromethyl)sulfanyl]indolizin-8-amine
2-(3-{[2-(dimethylphosphoryl)-4-methoxypyrimidin-5-yl]amino}prop-1-yn-1-yl)-N-[(3S,4R)-3-fluoro-1-methylpiperidin-4-yl]-3-[(trifluoromethyl)sulfanyl]indolizin-8-amine
2-(3-{[6-(dimethylphosphoryl)pyridazin-3-yl]amino}prop-yn-1-yl)-N-[(3S,4R)-3-fluoro-1-methylpiperidin-4-yl]-3-[(trifluoromethyl)sulfanyl]indolizin-8-amine
7-(dimethylphosphoryl)-N-[3-(8-{[(3S,4R)-3-fluoro-1-methylpiperidin-4-yl]amino}-3-[(trifluoromethyl)sulfanyl]indolizin-2-yl)prop-2-yn-1-yl]-1,3-benzoxazol-4-amine
7-(dimethylphosphoryl)-N-[3-(8-{[(3R,4S)-3-fluoro-1-methylpiperidin-4-yl]amino}-3-[(trifluoromethyl)sulfanyl]indolizin-2-yl)prop-2-yn-1-yl]-1,3-benzothiazol-4-amine
8-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-methyl-[1,2,4]triazolo[4,3-a]pyridine-6-carboxamide
4-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-[(2R*)-2-hydroxy(2-2H)propyl]-3-methoxybenzamide
4-{[3-(8-{[(3S,4R)-3-fluoro-1-methylpiperidin-4-yl]amino}-3-[(trifluoromethyl)sulfanyl]indolizin-2-yl)prop-2-yn-1-yl]amino}-N-[(2R*)-2-hydroxy(2-2H)propyl]-3-methoxy benzamide
4-((3-(8-(((3S,4R)-3-fluoro-1-(methyl-d3)piperidin-4-yl)amino)-3-((trifluoromethyl)thio)imidazo[1,2-a]pyridin-2-yl)prop-2-yn-1-yl)amino)-3-methoxy-N-methylbenzamide
4-((3-(8-(((3S,4R)-3-fluoro-1-methylpiperidin-4-yl)amino)-3-((trifluoromethyl)thio)indolizin-2-yl)prop-2-yn-1-yl)amino)-3-(methoxy-d3)-N-methylbenzamide
4-{[3-(8-{[(3S,4R)-3-fluoro-1-methylpiperidin-4-yl]amino}-3-[(trifluoromethyl)sulfanyl]imidazol 1,2-a]pyridin-2-yl)prop-2-yn-1-yl]amino)-3-(H3)methoxy-N-methylbenzamide
3-((3-(1-(((3S,4R)-3-fluoro-1-methylpiperidin-4-yl)amino)-6-((trifluoromethyl)thio)pyrrolo[1,2-a]pyrazin-7-yl)prop-2-yn-1-yl)amino)-4-(methoxy-d3)-N-methylbenzamide. or
3-{[3-(7-{[(3S,4R)-3-fluoro-1-methylpiperidin-4-yl]amino}-3-[(trifluoromethyl)sulfanyl]pyrazolo[1,5-a]pyridin-2-yl)prop-2-yn-1-yl]amino}-4-(2H3)methoxy-N-methylbenzamide.

Compounds herein can include all stereoisomers, enantiomers, diastereomers, mixtures, racemates, atropisomers, and tautomers thereof.

Non-limiting examples of optional substituents include a hydroxyl group, an oxo group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, carbamoyl group, amide group, phosphine group, phosphine oxide group, ureido group, epoxy group, and ester group.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, trifluoroethyl, aminomethyl, 1-chloroethyl, 2-hydroxyethyl, 1,2-difluoroethyl, and 3-carboxypropyl.

Non-limiting examples of amide groups include —C(O)NH₂, —C(O)N(H)CH₃, —C(O)N(H)CH₂CH₃, —C(O)N(CH₂CH₃)₂, —C(O)N(CH₃)₂, —C(O)N(H)CH(CH₃)₂, and —C(O)N(H)C(CH₃)₃.

Non-limiting examples of sulfonamide groups include —S(O)₂NH₂, —S(O)₂N(H)CH₃, —S(O)₂N(H)CH₂CH₃, —S(O)₂N(CH₂CH₃)₂, —S(O)₂N(CH₃)₂, —S(O)₂N(H)CH(CH₃)₂, and —S(O)₂N(H)C(CH₃)₃.

Non-limiting examples of sulfone groups include —S(O)₂CH₃, —S(O)₂CH₂CH₃, —S(O)₂CH(CH₃)₂, and —S(O)₂C(CH₃)₃.

Non-limiting examples of cyclic alkyl groups include cyclopropyl, cyclobutyl, cyclopentyl, cyclohexyl, cycloheptyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2,2,2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, 2-methoxy-4-(methylsulfonyl)phenyl, 2-methoxy-4-(ethylsulfonyl)phenyl, 2-methoxy-4-(ethylcarbamoyl)phenyl, 2-methoxy-4-(methylcarbamoyl)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

A heterocyclylalkyl group (e.g. “heterocyclylalkyl”) can be a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocyclylalkyl radical can be a monocyclic, bicyclic, tricyclic, or tetracyclic ring system, which may include fused (when fused with an aryl or a heteroaryl ring, the heterocyclylalkyl is bonded through a non-aromatic ring atom), spirocyclic, or bridged ring systems. The nitrogen, phosphorous, carbon, or sulfur atoms in the heterocyclylalkyl radical may be optionally oxidized. For example, a cyclic phosphane group (e.g., 1-methylphospinane) can be substituted with an oxo group to afford the corresponding phosphine oxide (e.g. 1-methylphospinane 1-oxide), or substituted with a ═N—R^{N} radical to afford the corresponding phosphinanimine (e.g. 1-methyl-1-phosphinan-1-imine), where R^{N} is any suitable monovalent radical. The nitrogen atom in the heterocyclylalkyl radical can be optionally quaternized.

Representative heterocyclylalkyls include, but are not limited to, heterocyclylalkyls having from two to fifteen carbon atoms (C₂-C₁₅ heterocyclylalkyl), from two to ten carbon atoms (C₂-C₁₀ heterocyclylalkyl), from two to eight carbon atoms (C₂-C₈ heterocyclylalkyl), from two to six carbon atoms (C₂-C₆ heterocyclylalkyl), from two to five carbon atoms (C₂-C₅ heterocyclylalkyl), or two to four carbon atoms (C₂-C₄ heterocyclylalkyl). In some embodiments, the heterocyclylalkyl is a 3- to 6-membered heterocyclylalkyl. In some embodiments, the cycloalkyl is a 5- to 6-membered heterocyclylalkyl. Examples of such heterocyclylalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocyclylalkyl also includes all ring forms of the carbohydrates, including but not limited to, the monosaccharides, the disaccharides, and the oligosaccharides. It is understood that when referring to the number of carbon atoms in a heterocyclylalkyl, the number of carbon atoms in the heterocyclylalkyl is not the same as the total number of atoms (including the heteroatoms) that make up the heterocyclylalkyl (i.e. skeletal atoms of the heterocyclylalkyl ring). Unless stated otherwise specifically in the specification, a heterocyclylalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heterocyclylalkyl is optionally substituted with oxo, halogen, methyl, ethyl, —CN, —CF₃, —OH, —OMe, —NH₂, or —NO₂. In some embodiments, a heterocyclylalkyl is optionally substituted with oxo, halogen, methyl, ethyl, —CN, —CF₃, —OH, or —OMe. In some embodiments, the heterocyclylalkyl is optionally substituted with halogen.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, compounds of the invention can be used to treat cancer in a subject. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilms tumor. In some embodiments, the solid tumor is selected from small cell lung cancer, pancreatic cancer, prostate cancer, breast cancer, endornetrial cancer, and ovarian cancer.

In some embodiments, the compounds of the invention show non-lethal toxicity.

### Pharmaceutically-Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, pyrazine, or pyrimidine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, an imidazole salt, a pyrazine salt, or a pyrimidine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions of the Invention.

A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the invention can be applied topically to the skin, or a body cavity, for example, oral, vaginal, bladder, cranial, spinal, thoracic, or pelvic cavity of a subject. The compounds of the invention can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein (e.g. a compound that binds a mutant p53 protein and increases wild-type p53 activity of the mutant protein) can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein (e.g. a compound that binds a mutant p53 protein and increases wild-type p53 activity of the mutant protein) can be present in a composition in a range of from about 1 mg to about 5000 mg; from about 100 mg to about 4000 mg; from about 10 mg to about 3500 mg; from about 5 mg to about 3000 mg, from about 10 mg to about 2500 mg, from about 50 mg to about 2000 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg. In some embodiments, the therapeutically-effective amount of the compound is from about 250 mg to about 2500 mg. In some embodiments, the therapeutically-effective amount of the compound is from about 500 mg to about 2000 mg. In some embodiments, the therapeutically-effective amount of the compound is from about 1000 mg to about 3000 mg. In some embodiments, the therapeutically-effective amount of the compound is from about 2000 mg to about 3000 mg. A compound described herein (e.g. a compound that binds a mutant p53 protein and increases wild-type p53 activity of the mutant protein) can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg. In some embodiments, the therapeutically-effective amount of the compound is about 2250 mg, about 2500 mg, or about 3000 mg. In some embodiments, the therapeutically-effective amount of the compound is about 1,200 mg. In some embodiments, the therapeutically-effective amount of the compound is about 1,500 m. In some embodiments, the therapeutically-effective amount of the compound is about 2,000 mg. In some embodiments, the therapeutically-effective amount of the compound is about 2,500 mg. In some embodiments, the therapeutically-effective amount of the compound is about 3,000 mg. In some embodiments, the therapeutically-effective amount of the compound is about 3,500 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

### Combination Treatment

Combination therapy with a compound of the disclosure (e.g. a compound that binds a mutant p53 protein and increases wild-type p53 activity of the mutant protein) and at least one additional therapeutic agent, for example, a MDM2 inhibitor, can be used to treat a condition. In some embodiments, the combination therapy can produce a significantly better therapeutic result than the additive effects achieved by each individual constituent when administered alone at a therapeutic dose. In some embodiments, the dosage of a MDM2 inhibitor in combination therapy can be reduced as compared to monotherapy with each agent, while still achieving an overall therapeutic effect. In some embodiments, a compound and a MDM2 inhibitor can exhibit a synergistic effect. In some embodiments, the synergistic effect of a compound a MDM2 inhibitor can be used to reduce the total amount drugs administered to a subject, which decrease side effects experienced by the subject.

Accordingly, in one aspect, the present disclosure provides a method for treating cancer, the method comprising administering to a subject in need thereof (a) an effective amount of a compound of the disclosure and (b) an effective amount of a MDM2 inhibitor to provide a combination therapy. In some embodiments, the combination therapy may have an enhanced therapeutic effect compared to the effect of the compound and the at least one additional pharmaceutically active agent each administered alone. According to certain exemplary embodiments, the combination therapy has a synergistic therapeutic effect. According to this embodiment, the combination therapy produces a significantly better therapeutic result (e.g., anti-cancer, cell growth arrest, apoptosis, induction of differentiation, cell death, etc.) than the additive effects achieved by each individual constituent when administered alone at a therapeutic dose.

Combination therapy includes but is not limited to the combination of compounds of the disclosure with chemotherapeutic agents, therapeutic antibodies, or radiation treatment, to provide a synergistic therapeutic effect. In some embodiments, the compounds of the disclosure are used in combination with one or more anti-cancer (antineoplastic or cytotoxic) chemotherapy drug. Suitable chemotherapeutic agents for use in the combinations of the present disclosure include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, or any combination thereof.

a. Combination Treatment with MDM2 Inhibitors

Disclosed herein are pharmaceutical compositions comprising a MDM2 inhibitor. Also disclosed herein are methods of treating a condition in a subject in need thereof comprising administering a MDM2 inhibitor. In some embodiments, the MDM2 inhibitor is Nutlin-1, Nutlin-2, Nutlin-3, RG7112 (RO5045337), idasanutlin (RG7388), AMG-232 (KRT-232), APG-115, BI-907828, CGM097, siremadlin (HDM201), SAR405838 (MI-77301), MK-8242 (SCH 900242), RAIN-32, or milademetan (DS-3032b), or a pharmaceutically-acceptable salt of any of the foregoing:

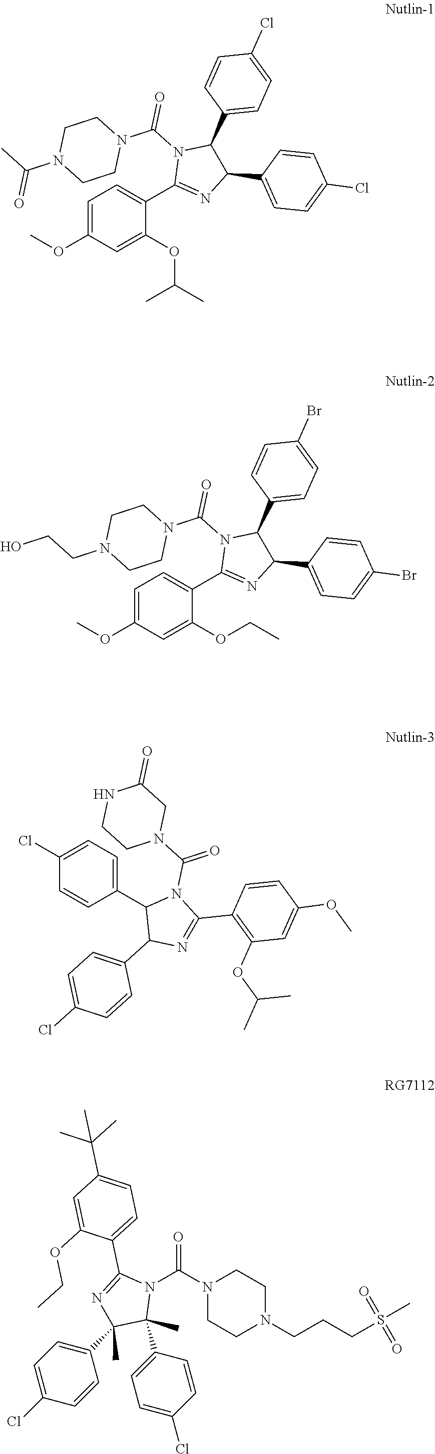 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1 ; CCOc1cc(OC)ccc1C1=N[C@H](c2ccc(Br)cc2)[C@H](c2ccc(Br)cc2)N1C(=O)N1CCN(CCO)CC1 ; COc1ccc(C2=NC(c3ccc(Cl)cc3)C(c3ccc(Cl)cc3)N2C(=O)N2CCNC(=O)C2)c(OC(C)C)c1 ; CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

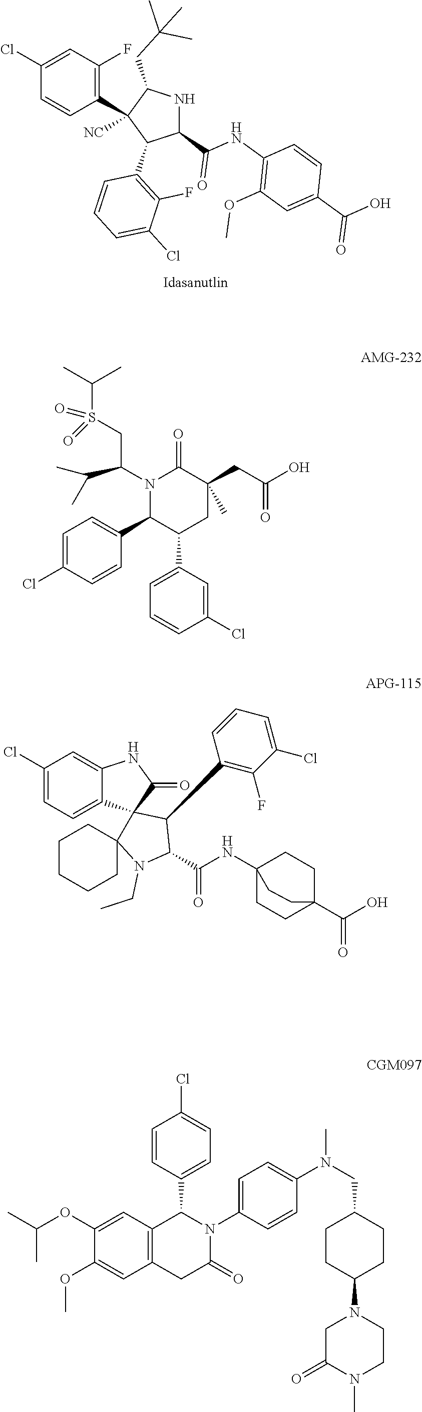 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F ; CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

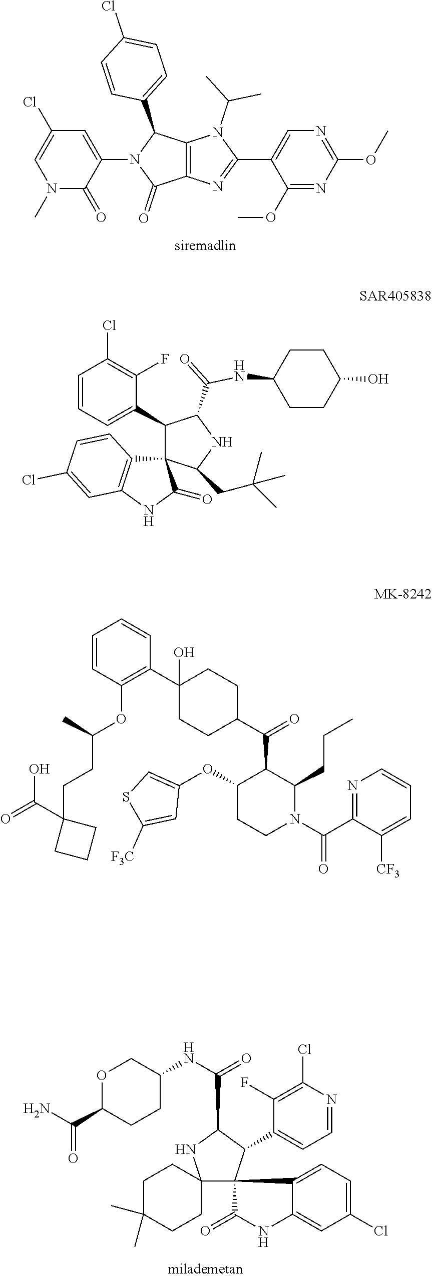 [SMILES: COc1ncc(-c2nc3c(n2C(C)C)[C@H](c2ccc(Cl)cc2)N(c2cc(Cl)cn(C)c2=O)C3=O)c(OC)n1 ; CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12 ; CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F ; CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

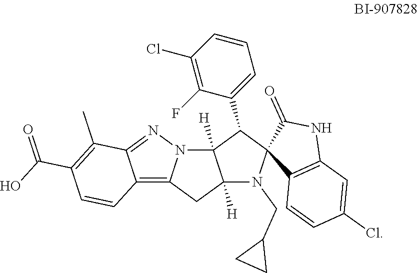 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

MDM2 inhibitors of the present disclosure also include stereoisomers of MDM2 inhibiting structures disclosed herein, including diastereomers, epimers, and enantiomers thereof. For example, Nutlin-3 includes Nutlin-3a (i.e., ((−)-Nutlin-3), or 4-[[(4S,5R)-4,5-bis(4-chlorophenyl)-2-(4-methoxy-2-propan-2-yloxyphenyl)-4,5-dihydroimidazol-1-yl]-oxomethyl]-2-piperazinone) and Nutlin-3b (i.e., ((+)-Nutlin-3), or 4-[[(4R,5S)-4,5-bis(4-chlorophenyl)-2-(4-methoxy-2-propan-2-yloxyphenyl)-4,5-dihydroimidazol-1-yl]-oxomethyl]-2-piperazinone).

b. Combination Treatment with PI3K Inhibitors

Disclosed herein are pharmaceutical compositions comprising a PI3K inhibitor. In some embodiments, the PI3K inhibitor is selected from idelalisib, copanlisib, duvelisib, alpelisib, seletalisib, gedatolisib, inavolisib, rigosertib sodium, leniolisib, umbralisib, buparlisib (AN2025), AMG-319, GM -604, acalisib, bimiralisib, GDC-0084, ACP-319, tenalisib, serabelisib, SF-1126, nemiralisib, fimepinostat, LY-3023414, voxtalisib, dactolisib, eganelisib, parsaclisib, GSK2636771, AZD-8186, and ASN-003. In some embodiments, the PI3K inhibitor is selected from a PI3Kα, PI3Kβ, PI3Kγ, or PI3Kδ inhibitor. Also disclosed herein are methods of treating a condition in a subject in need thereof comprising administering a PI3KT 10u inhibitor. In some embodiments, the PI3K inhibitor is selected from alpelisib, inavolisib, serabelisib, GSK2636771, eganelisib, idelalisib, or duvelisib.

In some embodiments, the PI3K inhibitor is:

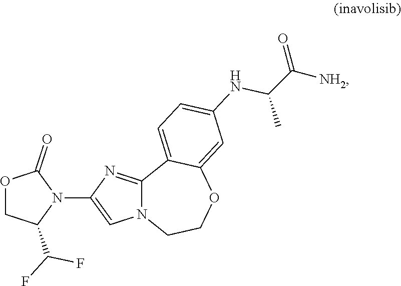 [SMILES: C[C@H](Nc1ccc2c(c1)OCCn1cc(N3C(=O)OC[C@H]3C(F)F)nc1-2)C(N)=O]

or a pharmaceutically-acceptable salt thereof.

In some embodiments, the PI3K inhibitor is:

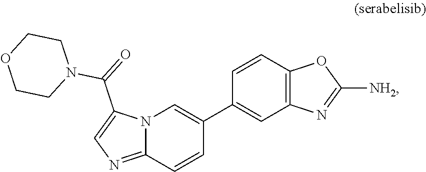 [SMILES: Nc1nc2cc(-c3ccc4ncc(C(=O)N5CCOCC5)n4c3)ccc2o1]

or a pharmaceutically-acceptable salt thereof.

In some embodiments, the PI3K inhibitor is:

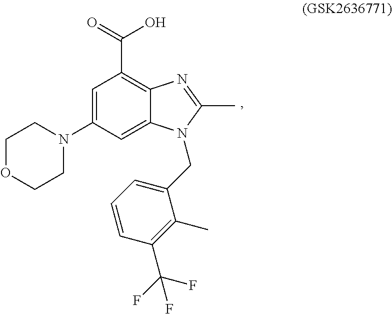 [SMILES: Cc1c(Cn2c(C)nc3c(C(=O)O)cc(N4CCOCC4)cc32)cccc1C(F)(F)F]

or a pharmaceutically-acceptable salt thereof.

In some embodiments, the PI3K inhibitor is:

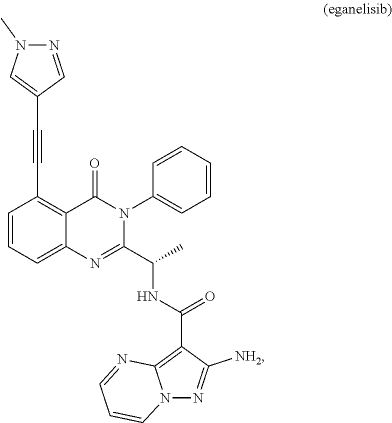 [SMILES: C[C@H](NC(=O)c1c(N)nn2cccnc12)c1nc2cccc(C#Cc3cnn(C)c3)c2c(=O)n1-c1ccccc1]

or a pharmaceutically-acceptable salt thereof.

In some embodiments, the PI3K inhibitor is:

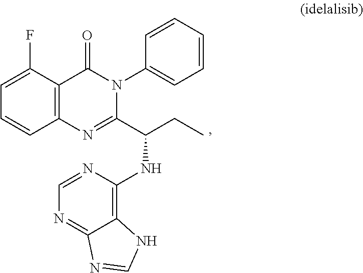 [SMILES: CC[C@H](Nc1ncnc2nc[nH]c12)c1nc2cccc(F)c2c(=O)n1-c1ccccc1]

or a pharmaceutically-acceptable salt thereof.

In some embodiments, the PI3K inhibitor is:

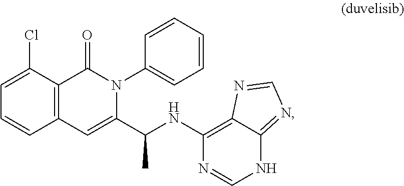 [SMILES: C[C@H](Nc1nc[nH]c2ncnc1-2)c1cc2cccc(Cl)c2c(=O)n1-c1ccccc1]

or a pharmaceutically-acceptable salt thereof.

In some embodiments, the PI3K inhibitor is

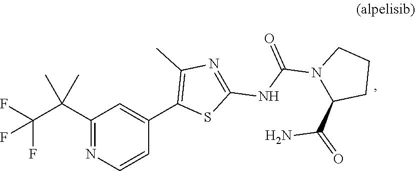 [SMILES: Cc1nc(NC(=O)N2CCC[C@H]2C(N)=O)sc1-c1ccnc(C(C)(C)C(F)(F)F)c1]

or a pharmaceutically-acceptable salt thereof.
c. Combination Treatment with AKT Inhibitors

Disclosed herein are pharmaceutical compositions comprising an AKT inhibitor. In some embodiments, the AKT inhibitor is selected from the AKT inhibitor is selected from the group consisting of A6730, B2311, 124018, GSK2110183 (afuresertib), Perifosine (KRX-0401), GDC-0068 (ipatasertib), RX-0201, VQD-002, LY294002, A-443654, A-674563, Akti-1, Akti-2, Akti-1/2, AR-42, API-59CJ-OMe, ATI-13148, AZD-5363, erucylphosphocholine, GSK-2141795 (GSK795), MK2206 (CAS #1032349-93-1), KP372-1, L-418, NL-71-101, PBI-05204, PIA5, PX-316, SR13668, triciribine, GSK 690693 (CAS #937174-76-0), FPA 124 (CAS #902779-59-3), Miltefosine, capivasertib, PHT-427 (CAS #1 191951-57-1), 10-DEBC hydrochloride, Akt inhibitor III, Akt inhibitor VIII, MK-2206 dihydrochloride (CAS #1032350-13-2), SC79, AT7867 (CAS #857531-00-1), CCT128930 (CAS #885499-61-6), A-674563 (CAS #552325-73-2), AGL 2263, AS-041 164 (5-benzo[1,3]dioxol-5-ylmethylene-thiazolidine-2,4-dione), BML-257 (CAS #32387-96-5), XL-418, CAS #612847-09-3, CAS #98510-80-6, H-89 (CAS #127243-85-0), OXY-1 1 1 A, and 3-[1-[[4-(7-phenyl-3H-imidazo[4,5-g]quinoxalin-6-yl)phenyl]methyl]piperidin-4-yl]-1H-benzimidazol-2-one. Also disclosed herein are methods of treating a condition in a subject in need thereof comprising administering an AKT inhibitor. In some embodiments, the AKT inhibitor is selected from MK2206, ipatasertib, and capivasertib, or a pharmaceutically-acceptable salt thereof. In some embodiments, the AKT inhibitor is an inhibitor of Akt1.

In some embodiments, the AKT inhibitor is:

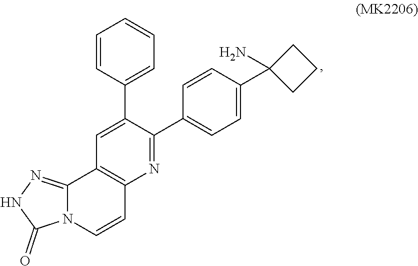 [SMILES: NC1(c2ccc(-c3nc4ccn5c(=O)[nH]nc5c4cc3-c3ccccc3)cc2)CCC1]

or a pharmaceutically-acceptable salt thereof.

In some embodiments, the AKT inhibitor is:

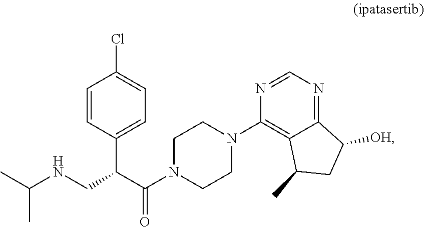 [SMILES: CC(C)NC[C@@H](C(=O)N1CCN(c2ncnc3c2[C@H](C)C[C@H]3O)CC1)c1ccc(Cl)cc1]

or a pharmaceutically-acceptable salt thereof.

In some embodiments, the AKT inhibitor is:

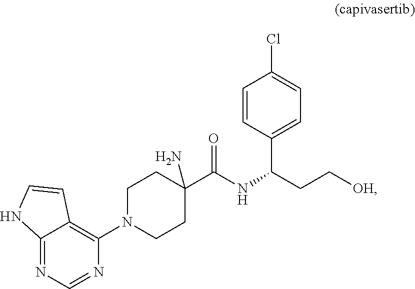 [SMILES: NC1(C(=O)N[C@@H](CCO)c2ccc(Cl)cc2)CCN(c2ncnc3[nH]ccc23)CC1]

or a pharmaceutically-acceptable salt thereof.

### Administration of Combination Treatment

A compound of the disclosure or a pharmaceutical composition comprising a compound of the disclosure and at a MDM2 inhibitor can be administered simultaneously (i.e., simultaneous administration) or sequentially (i.e., sequential administration). A compound of the disclosure or a pharmaceutical composition comprising a compound of the disclosure and a PI3K or AKT inhibitor can be administered simultaneously (i.e., simultaneous administration) or sequentially (i.e., sequential administration).

In some embodiments, a compound of the disclosure and the MDM2 inhibitor are administered simultaneously, either in the same composition or in separate compositions. When the drugs are administered simultaneously, the compound and the MDM2 inhibitor may be contained in the same composition (e.g., a composition comprising both the compound and MDM2 inhibitor) or in separate compositions (e.g., the compound is contained in one composition and the MDM2 inhibitor is contained in another composition). In some embodiments, a compound of the disclosure and the PI3K or AKT inhibitor are administered simultaneously, either in the same composition or in separate compositions. When the drugs are administered simultaneously, the compound and the PI3K or AKT inhibitor may be contained in the same composition (e.g., a composition comprising both the compound and PI3K or AKT inhibitor) or in separate compositions (e.g., the compound is contained in one composition and the PI3K or AKT inhibitor is contained in another composition).

In some embodiments, the compound and the MDM2 inhibitor are administered sequentially, i.e., the compound is administered either prior to or after the administration of the additional pharmaceutically-active agent. In some embodiments, the compound is administered before the MDM2 inhibitor. In some embodiments, the MDM2 inhibitor, is administered before the compound. The compound and the MDM2 inhibitor are contained in separate compositions, which may be contained in the same or different packages. In some embodiments, the compound and the PI3K or AKT inhibitor are administered sequentially, i.e., the compound is administered either prior to or after the administration of the additional pharmaceutically-active agent. In some embodiments, the compound is administered before the PI3K or AKT inhibitor. In some embodiments, the PI3K or AKT inhibitor, is administered before the compound. The compound and the PI3K or AKT inhibitor are contained in separate compositions, which may be contained in the same or different packages.

In some embodiments, the administration of the compounds and the MDM2 inhibitor are concurrent, i.e., the administration period of the compounds and that of the agent overlap with each other. In some embodiments, the administration of the compounds and the MDM2 inhibitor are non-concurrent. For example, in some embodiments, the administration of the compound is terminated before the MDM2 inhibitor is administered. In some embodiments, the administration of the MDM2 inhibitor is terminated before the compound is administered. The time period between these two non-concurrent administrations can range from being days apart to being weeks apart. In some embodiments, the administration of the compounds and the PI3K or AKT inhibitor are concurrent, i.e., the administration period of the compounds and that of the agent overlap with each other. In some embodiments, the administration of the compounds and the PI3K or AKT inhibitor are non-concurrent. For example, in some embodiments, the administration of the compound is terminated before the PI3K or AKT inhibitor is administered. In some embodiments, the administration of the PI3K or AKT inhibitor is terminated before the compound is administered. The time period between these two non-concurrent administrations can range from being days apart to being weeks apart.

The dosing frequency of the compound and the MDM2 inhibitor may be adjusted over the course of the treatment, based on the judgment of the administering physician. When administered separately, the compound and the MDM2 inhibitor can be administered at different dosing frequency or intervals. For example, the compound can be administered weekly, while the at least one MDM2 inhibitor can be administered more or less frequently. Or, the compound can be administered twice weekly, while MDM2 inhibitor can be administered more or less frequently. In addition, the compound and the MDM2 inhibitor can be administered using the same route of administration or using different routes of administration. The dosing frequency of the compound and the PI3K or AKT inhibitor may be adjusted over the course of the treatment, based on the judgment of the administering physician. When administered separately, the compound and the PI3K or AKT inhibitor can be administered at different dosing frequency or intervals. For example, the compound can be administered weekly, while the at least one PI3K or AKT inhibitor can be administered more or less frequently. Or, the compound can be administered twice weekly, while PI3K or AKT inhibitor can be administered more or less frequently. In addition, the compound and the PI3K or AKT inhibitor can be administered using the same route of administration or using different routes of administration.

A therapeutically-effective amount of a compound and/or MDM2 inhibitor for use in therapy can vary with the nature of the condition being treated, the length of treatment time desired, the age and the condition of the patient, and can be determined by the attending physician. A therapeutically-effective amount of a compound and/or PI3K or AKT inhibitor for use in therapy can vary with the nature of the condition being treated, the length of treatment time desired, the age and the condition of the patient, and can be determined by the attending physician.

In some embodiments, when a compound of the disclosure is administered in combination with the MDM2 inhibitor, the dosage of the compound can be given a lower dosage than when the compound is administered alone. In some embodiments, the dosage of a compound of the disclosure in combination therapy can be from about 5 mg/kg to about 1000 mg/kg; from about 50 mg/kg to about 600 mg/kg; from about 150 mg/kg to about 600 mg/kg; or from about 300 mg/kg to about 600 mg/kg. In some embodiments, the dosage of a compound of the disclosure in combination therapy can be from about 5 mg/kg to about 25 mg/kg; from about 25 mg/kg to about 50 mg/kg; from about 50 mg/kg to about 75 mg/kg; from about 75 mg/kg to about 100 mg/kg; from about 100 mg/kg to about 125 mg/kg; from about 125 mg/kg to about 150 mg/kg; from about 150 mg/kg to about 200 mg/kg; from about 200 mg/kg to about 250 mg/kg; from about 250 mg/kg to about 300 mg/kg; from about 300 mg/kg to about 350 mg/kg; from about 350 mg/kg to about 400 mg/kg; from about 400 mg/kg to about 450 mg/kg; from about 450 mg/kg to about 500 mg/kg; from about 500 mg/kg to about 550 mg/kg; from about 550 mg/kg to about 600 mg/kg; from about 600 mg/kg to about 700 mg/kg; from about 700 mg/kg to about 800 mg/kg; from about 800 mg/kg to about 900 mg/kg; or from about 900 mg/kg to about 1000 mg/kg. In some embodiments, the dosage of a compound of the disclosure in combination therapy can be from about 50 mg/kg to about 75 mg/kg. In some embodiments, the dosage of a compound of the disclosure in combination therapy can be from about 75 mg/kg to about 150 mg/kg. In some embodiments, the dosage of a compound of the disclosure in combination therapy can be from about 150 mg/kg to about 300 mg/kg. In some embodiments, the dosage of a compound of the disclosure in combination therapy can be from about 300 mg/kg to about 600 mg/kg. In some embodiments, when a compound of the disclosure is administered in combination with the PI3K or AKT inhibitor, the dosage of the compound can be given a lower dosage than when the compound is administered alone. In some embodiments, the dosage of a compound of the disclosure in combination therapy can be from about 5 mg/kg to about 1000 mg/kg; from about 50 mg/kg to about 600 mg/kg; from about 150 mg/kg to about 600 mg/kg; or from about 300 mg/kg to about 600 mg/kg. In some embodiments, the dosage of a compound of the disclosure in combination therapy can be from about 5 mg/kg to about 25 mg/kg; from about 25 mg/kg to about 50 mg/kg; from about 50 mg/kg to about 75 mg/kg; from about 75 mg/kg to about 100 mg/kg; from about 100 mg/kg to about 125 mg/kg; from about 125 mg/kg to about 150 mg/kg; from about 150 mg/kg to about 200 mg/kg; from about 200 mg/kg to about 250 mg/kg; from about 250 mg/kg to about 300 mg/kg; from about 300 mg/kg to about 350 mg/kg; from about 350 mg/kg to about 400 mg/kg; from about 400 mg/kg to about 450 mg/kg; from about 450 mg/kg to about 500 mg/kg; from about 500 mg/kg to about 550 mg/kg; from about 550 mg/kg to about 600 mg/kg; from about 600 mg/kg to about 700 mg/kg; from about 700 mg/kg to about 800 mg/kg; from about 800 mg/kg to about 900 mg/kg; or from about 900 mg/kg to about 1000 mg/kg. In some embodiments, the dosage of a compound of the disclosure in combination therapy can be from about 50 mg/kg to about 75 mg/kg. In some embodiments, the dosage of a compound of the disclosure in combination therapy can be from about 75 mg/kg to about 150 mg/kg. In some embodiments, the dosage of a compound of the disclosure in combination therapy can be from about 150 mg/kg to about 300 mg/kg. In some embodiments, the dosage of a compound of the disclosure in combination therapy can be from about 300 mg/kg to about 600 mg/kg.

In some embodiments, the dosage of a compound of the disclosure in combination therapy can be in an amount of about 5 mg/kg; about 25 mg/kg; about 50 mg/kg; about 75 mg/kg; about 100 mg/kg; about 125 mg/kg; about 150 mg/kg; about 175 mg/kg; about 200 mg/kg; about 250 mg/kg; about 300 mg/kg; about 350 mg/kg; about 400 mg/kg; about 450 mg/kg; about 500 mg/kg; about 550 mg/kg; about 600 mg/kg; about 700 mg/kg; about 800 mg/kg; about 900 mg/kg; or about 1000 mg/kg. In some embodiments, the dosage of a compound of the disclosure in combination therapy can be in an amount of about 50 mg/kg. In some embodiments, the dosage of a compound of the disclosure in combination therapy can be in an amount of about 75 mg/kg. In some embodiments, the dosage of a compound of the disclosure in combination therapy can be in an amount of about 150 mg/kg. In some embodiments, the dosage of a compound of the disclosure in combination therapy can be in an amount of about 300 mg/kg. In some embodiments, the dosage of a compound of the disclosure in combination therapy can be in an amount of about 600 mg/kg.

In some embodiments, when a compound of the disclosure is administered in combination with an MDM2 inhibitor, the dosage of the MDM2 inhibitor can be given a lower dosage than when the compound is administered alone. In some embodiments, the dosage of the MDM2 inhibitor in combination therapy can be from about 1 mg/kg to about 50 mg/kg; from about 50 mg/kg to about 100 mg/kg; from about 100 mg/kg to about 150 mg/kg; from about 150 mg/kg to about 200 mg/kg; from about 1 mg/kg to about 30 mg/kg; from about 1 mg/kg to about 50 mg/kg; from about 1 mg/kg to about 100 mg/kg; from about 10 mg/kg to about 30 mg/kg; from about 40 mg/kg to about 60 mg/kg; from about 1 mg/kg to about 25 mg/kg; from about 25 mg/kg to about 50 mg/kg; from about 50 mg/kg to about 75 mg/kg; from about 75 mg/kg to about 100 mg/kg; or from about 1 mg/kg to about 200 mg/kg. In some embodiments, the dosage of the MDM2 inhibitor in combination therapy can be from about 10 mg/kg to about 50 mg/kg. In some embodiments, when a compound of the disclosure is administered in combination with an PI3K or AKT inhibitor, the dosage of the MDM-2 inhibitor can be given a lower dosage than when the compound is administered alone. In some embodiments, the dosage of the PI3K or AKT inhibitor in combination therapy can be from about 1 mg/kg to about 50 mg/kg; from about 50 mg/kg to about 100 mg/kg; from about 100 mg/kg to about 150 mg/kg; from about 150 mg/kg to about 200 mg/kg; from about 1 mg/kg to about 30 mg/kg; from about 1 mg/kg to about 50 mg/kg; from about 1 mg/kg to about 100 mg/kg; from about 10 mg/kg to about 30 mg/kg; from about 40 mg/kg to about 60 mg/kg; from about 1 mg/kg to about 25 mg/kg; from about 25 mg/kg to about 50 mg/kg; from about 50 mg/kg to about 75 mg/kg; from about 75 mg/kg to about 100 mg/kg; or from about 1 mg/kg to about 200 mg/kg. In some embodiments, the dosage of the MDM-2 inhibitor in combination therapy can be from about 10 mg/kg to about 50 mg/kg.

In some embodiments, the dosage of the MDM2 inhibitor in combination therapy can be in an amount of about 1 mg/kg; about 5 mg/kg; about 10 mg/kg; about 20 mg/kg; about 30 mg/kg; about 40 mg/kg; about 50 mg/kg; about 60 mg/kg; about 70 mg/kg; about 80 mg/kg; about 90 mg/kg; or about 100 mg/kg. In some embodiments, the dosage of the MDM2 inhibitor in combination therapy can be in an amount of about 20 mg/kg. In some embodiments, the dosage of the PI3K or AKT inhibitor in combination therapy can be in an amount of about 1 mg/kg; about 5 mg/kg; about 10 mg/kg; about 20 mg/kg; about 30 mg/kg; about 40 mg/kg; about 50 mg/kg; about 60 mg/kg; about 70 mg/kg; about 80 mg/kg; about 90 mg/kg; or about 100 mg/kg. In some embodiments, the dosage of the PI3K or AKT inhibitor in combination therapy can be in an amount of about 20 mg/kg.

In some embodiments, the dosage of the MDM2 inhibitor in combination therapy can be from about 1 mg to about 5 mg; from about 5 mg to about 25 mg; from about 25 mg to about 50 mg; from about 50 mg to about 75 mg; from about 75 mg to about 100 mg; from about 100 mg to about 150 mg; from about 150 mg to about 200 mg; from about 200 mg to about 250 mg; from about 250 mg to about 300 mg; from about 300 mg to about 350 mg; from about 350 mg to about 400 mg; from about 400 mg to about 450 mg; from about 450 mg to about 500 mg; from about 500 mg to about 750 mg; from about 750 mg to about 1000 mg; from about 1000 mg to about 1250 mg; from about 750 mg to about 1250 mg; from about 500 mg to about 1500 mg; from about 500 mg to about 2000 mg; or from about 500 mg to about 2500 mg. In some embodiments, the dosage of the PI3K or AKT inhibitor in combination therapy can be from about 1 mg to about 5 mg; from about 5 mg to about 25 mg; from about 25 mg to about 50 mg; from about 50 mg to about 75 mg; from about 75 mg to about 100 mg; from about 100 mg to about 150 mg; from about 150 mg to about 200 mg; from about 200 mg to about 250 mg; from about 250 mg to about 300 mg; from about 300 mg to about 350 mg; from about 350 mg to about 400 mg; from about 400 mg to about 450 mg; from about 450 mg to about 500 mg; from about 500 mg to about 750 mg; from about 750 mg to about 1000 mg; from about 1000 mg to about 1250 mg; from about 750 mg to about 1250 mg; from about 500 mg to about 1500 mg; from about 500 mg to about 2000 mg; or from about 500 mg to about 2500 mg.

In some embodiments, the dosage of the compound of the disclosure administered in combination therapy can be from about 10 mg/kg to about 600 mg/kg; and the dosage of the MDM2 inhibitor in combination therapy can be from about 1 mg/kg to about 500 mg/kg. In some embodiments, the dosage of the compound of the disclosure administered in combination therapy can be from about 10 mg/kg to about 150 mg/kg; and the dosage of the MDM2 inhibitor in combination therapy can be from about 1 mg/kg to about 100 mg/kg. In some embodiments, the dosage of the compound of the disclosure administered in combination therapy can be from about 10 mg/kg to about 600 mg/kg; and the dosage of MDM2 inhibitor can be from about 1 mg/kg to about 200 mg/kg. In some embodiments, the dosage of the compound of the disclosure administered in combination therapy can be from about 10 mg/kg to about 600 mg/kg; and the dosage of the PI3K or AKT inhibitor in combination therapy can be from about 1 mg/kg to about 500 mg/kg. In some embodiments, the dosage of the compound of the disclosure administered in combination therapy can be from about 10 mg/kg to about 150 mg/kg; and the dosage of the PI3K or AKT inhibitor in combination therapy can be from about 1 mg/kg to about 100 mg/kg. In some embodiments, the dosage of the compound of the disclosure administered in combination therapy can be from about 10 mg/kg to about 600 mg/kg; and the dosage of PI3K or AKT inhibitor can be from about 1 mg/kg to about 200 mg/kg.

In some embodiments, the dosage of the compound of the disclosure administered in combination therapy can be about 50 mg/kg; and the dosage of MDM2 inhibitor in combination therapy can about 20 mg/kg. In some embodiments, the dosage of the compound of the disclosure administered in combination therapy can be about 50 mg/kg; and the dosage of MDM2 inhibitor in combination therapy can about 50 mg/kg. In some embodiments, the dosage of the compound of the disclosure administered in combination therapy can be about 100 mg/kg; and the dosage of MDM2 inhibitor in combination therapy can about 20 mg/kg. In some embodiments, the dosage of the compound of the disclosure administered in combination therapy can be about 50 mg/kg; and the dosage of MDM2 inhibitor in combination therapy can about 10 mg/kg. In some embodiments, the dosage of the compound of the disclosure administered in combination therapy can be about 100 mg/kg; and the dosage of MDM2 inhibitor in combination therapy can about 50 mg/kg. In some embodiments, the dosage of the compound of the disclosure administered in combination therapy can be about 50 mg/kg; and the dosage of PI3K or AKT inhibitor in combination therapy can about 20 mg/kg. In some embodiments, the dosage of the compound of the disclosure administered in combination therapy can be about 50 mg/kg; and the dosage of PI3K or AKT inhibitor in combination therapy can about 50 mg/kg. In some embodiments, the dosage of the compound of the disclosure administered in combination therapy can be about 100 mg/kg; and the dosage of PI3K or AKT inhibitor in combination therapy can about 20 mg/kg. In some embodiments, the dosage of the compound of the disclosure administered in combination therapy can be about 50 mg/kg; and the dosage of PI3K or AKT inhibitor in combination therapy can about 10 mg/kg. In some embodiments, the dosage of the compound of the disclosure administered in combination therapy can be about 100 mg/kg; and the dosage of PI3K or AKT inhibitor in combination therapy can about 50 mg/kg.

In some embodiments, the dosage of the compound of the disclosure administered in combination therapy can be from about 500 mg to about 3000 mg; and the dosage of MDM2 inhibitor in combination therapy can be from about 10 mg/kg to about 50 mg/kg. In some embodiments, the dosage of the compound of the disclosure administered in combination therapy can be from about 500 mg to about 2000 mg; and the dosage of MDM2 inhibitor in combination therapy can be from about 10 mg/kg to about 50 mg/kg. In some embodiments, the dosage of the compound of the disclosure administered in combination therapy can be from about 500 mg to about 1500 mg; and the dosage of MDM2 inhibitor in combination therapy can be from about 10 mg/kg to about 50 mg/kg. In some embodiments, the dosage of the compound of the disclosure administered in combination therapy can be from about 500 mg to about 1000 mg; and the dosage of MDM2 inhibitor in combination therapy can be from about 10 mg/kg to about 50 mg/kg. In some embodiments, the dosage of the compound of the disclosure administered in combination therapy can be from about 500 mg to about 3000 mg; and the dosage of PI3K or AKT inhibitor in combination therapy can be from about 10 mg/kg to about 50 mg/kg. In some embodiments, the dosage of the compound of the disclosure administered in combination therapy can be from about 500 mg to about 2000 mg; and the dosage of PI3K or AKT inhibitor in combination therapy can be from about 10 mg/kg to about 50 mg/kg. In some embodiments, the dosage of the compound of the disclosure administered in combination therapy can be from about 500 mg to about 1500 mg; and the dosage of PI3K or AKT inhibitor in combination therapy can be from about 10 mg/kg to about 50 mg/kg. In some embodiments, the dosage of the compound of the disclosure administered in combination therapy can be from about 500 mg to about 1000 mg; and the dosage of MDM-2 inhibitor in combination therapy can be from about 10 mg/kg to about 50 mg/kg.

In some embodiments, the dosage of the compound of the disclosure administered in combination therapy can be from about 10 mg/kg to about 100 mg/kg; and the dosage of an MDM2 inhibitor as an additional pharmaceutically-active agent in combination therapy is about 1000 mg. In some embodiments, the dosage of the compound of the disclosure administered in combination therapy can be from about 10 mg/kg to about 100 mg/kg; and the dosage of an MDM2 inhibitor as an additional pharmaceutically-active agent in combination therapy is about 500 mg. In some embodiments, the dosage of the compound of the disclosure administered in combination therapy can be from about 10 mg/kg to about 50 mg/kg; and the dosage of an MDM2 inhibitor as an additional pharmaceutically-active agent in combination therapy is about 1000 mg. In some embodiments, the dosage of the compound of the disclosure administered in combination therapy can be from about 10 mg/kg to about 50 mg/kg; and the dosage of an MDM2 inhibitor as an additional pharmaceutically-active agent in combination therapy is about 500 mg. In some embodiments, the dosage of the compound of the disclosure administered in combination therapy can be from about 10 mg/kg to about 100 mg/kg; and the dosage of a PI3K or AKT inhibitor as an additional pharmaceutically-active agent in combination therapy is about 1000 mg. In some embodiments, the dosage of the compound of the disclosure administered in combination therapy can be from about 10 mg/kg to about 100 mg/kg; and the dosage of a PI3K or AKT inhibitor as an additional pharmaceutically-active agent in combination therapy is about 500 mg. In some embodiments, the dosage of the compound of the disclosure administered in combination therapy can be from about 10 mg/kg to about 50 mg/kg; and the dosage of a PI3K or AKT inhibitor as an additional pharmaceutically-active agent in combination therapy is about 1000 mg. In some embodiments, the dosage of the compound of the disclosure administered in combination therapy can be from about 10 mg/kg to about 50 mg/kg; and the dosage of a PI3K or AKT inhibitor as an additional pharmaceutically-active agent in combination therapy is about 500 mg.

In some embodiments, the dosage of the compound of the disclosure administered in combination therapy is about 50 mg/kg; and the dosage of an MDM2 inhibitor as an additional pharmaceutically-active agent in combination therapy is about 1000 mg. In some embodiments, the dosage of the compound of the disclosure administered in combination therapy is about 100 mg/kg; and the dosage of an MDM2 inhibitor as an additional pharmaceutically-active agent in combination therapy is about 500 mg. In some embodiments, the dosage of the compound of the disclosure administered in combination therapy is about 50 mg/kg; and the dosage of an MDM2 inhibitor as an additional pharmaceutically-active agent in combination therapy is about 500 mg. In some embodiments, the dosage of the compound of the disclosure administered in combination therapy is about 50 mg/kg; and the dosage of an MDM2 inhibitor as an additional pharmaceutically-active agent in combination therapy is about 200 mg. In some embodiments, the dosage of the compound of the disclosure administered in combination therapy is about 50 mg/kg; and the dosage of an MDM2 inhibitor as an additional pharmaceutically-active agent in combination therapy is about 100 mg. In some embodiments, the dosage of the compound of the disclosure administered in combination therapy is about 50 mg/kg; and the dosage of a PI3K or AKT inhibitor as an additional pharmaceutically-active agent in combination therapy is about 1000 mg. In some embodiments, the dosage of the compound of the disclosure administered in combination therapy is about 100 mg/kg; and the dosage of a PI3K or AKT inhibitor as an additional pharmaceutically-active agent in combination therapy is about 500 mg. In some embodiments, the dosage of the compound of the disclosure administered in combination therapy is about 50 mg/kg; and the dosage of a PI3K or AKT inhibitor as an additional pharmaceutically-active agent in combination therapy is about 500 mg. In some embodiments, the dosage of the compound of the disclosure administered in combination therapy is about 50 mg/kg; and the dosage of a PI3K or AKT inhibitor as an additional pharmaceutically-active agent in combination therapy is about 200 mg. In some embodiments, the dosage of the compound of the disclosure administered in combination therapy is about 50 mg/kg; and the dosage of a PI3K or AKT inhibitor as an additional pharmaceutically-active agent in combination therapy is about 100 mg.

In some embodiments, a pharmaceutically-acceptable amount of a compound of the disclosure can be administered to a subject 1, 2, 3, 4, or 5 times a day. In some embodiments, a pharmaceutically-acceptable amount of a compound of the disclosure can be administered to a subject once a day. In some embodiments, a pharmaceutically-acceptable amount of a compound of the disclosure can be administered to a subject twice a day. In some embodiments, a pharmaceutically-acceptable amount of a compound of the disclosure can be administered to a subject three times a day.

In some embodiments, a pharmaceutically-acceptable amount of a compound of the disclosure can be administered to a subject 1, 2, 3, 4, or 5 times a day once every 1, 2, 3, 4, 5, 6, or 7 days. In some embodiments, a pharmaceutically-acceptable amount of a compound of the disclosure can be administered to a subject once a day once every 3 days. In some embodiments, a pharmaceutically-acceptable amount of a compound of the disclosure can be administered to a subject twice a day once every 3 days. In some embodiments, a pharmaceutically-acceptable amount of a compound of the disclosure can be administered to a subject 1, 2, 3, 4, or 5 times a day once every 7 days. In some embodiments, a pharmaceutically-acceptable amount of a compound of the disclosure can be administered to a subject once a day once every 7 days. In some embodiments, a pharmaceutically-acceptable amount of a compound of the disclosure can be administered to a subject twice a day once every 7 days.

In some embodiments, a pharmaceutically-acceptable amount of a compound of the disclosure can be administered to a subject 1, 2, 3, 4, or 5 times a day once every 1, 2, 3, 4, 5, 6, or 7 days for 1 to 50 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 5 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 10 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 15 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 20 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 25 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 30 doses. In some embodiments, a pharmaceutically-acceptable amount of a compound of the disclosure can be administered to a subject 1, 2, 3, 4, or 5 times a day once every 1, 2, 3, 4, 5, 6, or 7 days for about 35 doses.

In some embodiments, a pharmaceutically-acceptable amount of a compound of the disclosure can be administered to a subject once a day every 7 days for about 5 doses. In some embodiments, a pharmaceutically-acceptable amount of a compound of the disclosure can be administered to a subject once a day every 7 days for about 10 doses. In some embodiments, a pharmaceutically-acceptable amount of a compound of the disclosure can be administered to a subject twice a day every 7 days for about 15 doses. In some embodiments, a pharmaceutically-acceptable amount of a compound of the disclosure can be administered to a subject once a day every 3 days for about 20 doses. In some embodiments, a pharmaceutically-acceptable amount of a compound of the disclosure can be administered to a subject once a day every 3 days for about 35 doses.

In some embodiments, a pharmaceutically-acceptable amount of a compound of the disclosure can be administered to a subject twice a day every 7 days for about 5 doses. In some embodiments, a pharmaceutically-acceptable amount of a compound of the disclosure can be administered to a subject twice a day every 7 days for about 15 doses.

### Pharmaceutical Compositions for Combination Treatment

According to certain embodiments, the compounds and the additional pharmaceutically-active agent, for example, a MDM2 inhibitor, are administered within a single pharmaceutical composition. In some embodiments, the compounds of the disclosure and the MDM2 inhibitor can be provided in a single unit dosage form for being taken together. According to some embodiments, the pharmaceutical composition further comprises pharmaceutically-acceptable diluents or carrier. According to certain embodiments, the compounds of the disclosure and the MDM2 inhibitor are administered within different pharmaceutical composition. In some embodiments, the compounds of the disclosure and the MDM2 inhibitor can be provided in a single unit dosage as separate entities (e.g., in separate containers) to be administered simultaneously or with a certain time difference.

In some embodiments, the compounds of the disclosure and the MDM2 inhibitor can be administered via the same route of administration. In some embodiments, the compounds of the disclosure and the MDM2 inhibitor can be administered via the different route of administration. In some embodiments, a compound of the disclosure and MDM2 inhibitor are administered orally. In some embodiments, a compound of the disclosure is administered orally, and the MDM2 inhibitor is not administered orally. In some embodiments, a compound of the disclosure is not administered orally, and the MDM2 inhibitor is administered orally. According to certain embodiments, the compounds and the additional pharmaceutically-active agent, for example, a PI3K or AKT inhibitor, are administered within a single pharmaceutical composition. In some embodiments, the compounds of the disclosure and a PI3K or AKT inhibitor can be provided in a single unit dosage form for being taken together. According to some embodiments, the pharmaceutical composition further comprises pharmaceutically-acceptable diluents or carrier. According to certain embodiments, the compounds of the disclosure and a PI3K or AKT inhibitor are administered within different pharmaceutical composition. In some embodiments, the compounds of the disclosure and a PI3K or AKT inhibitor can be provided in a single unit dosage as separate entities (e.g., in separate containers) to be administered simultaneously or with a certain time difference. In some embodiments, the compounds of the disclosure and a PI3K or AKT inhibitor can be administered via the same route of administration. In some embodiments, the compounds of the disclosure and a PI3K or AKT inhibitor can be administered via the different route of administration. In some embodiments, a compound of the disclosure and a PI3K or AKT inhibitor are administered orally. In some embodiments, a compound of the disclosure is administered orally, and a PI3K or AKT inhibitor is not administered orally. In some embodiments, a compound of the disclosure is not administered orally, and a PI3K or AKT inhibitor is administered orally.

Treatment of a condition by administering a compound of the disclosure in combination with an additional anti-cancer agent (e.g., MDM2 inhibitor) can increase a median survival time of a subject compared to subjects who do not receive combination therapy with a compound of the disclosure and the additional anti-cancer agent. In some embodiments, a median survival time of a first patient population receiving combination therapy with a compound of the disclosure and a MDM2 inhibitor can be greater than a median survival time of a second patient population that does not receive any cancer therapy. In some embodiments, a median survival time of a first patient population receiving combination therapy with a compound of the disclosure and a MDM2 inhibitor can be greater than a median survival time of a second patient population that receives therapy with a compound of the disclosure alone. In some embodiments, a median survival time of a first patient population receiving combination therapy with a compound of the disclosure and a MDM2 inhibitor can be greater than a median survival time of a second patient population that receives therapy with the MDM2 inhibitor alone. Treatment of a condition by administering a compound of the disclosure in combination with an additional anti-cancer agent (e.g., a PI3K or AKT inhibitor) can increase a median survival time of a subject compared to subjects who do not receive combination therapy with a compound of the disclosure and the additional anti-cancer agent. In some embodiments, a median survival time of a first patient population receiving combination therapy with a compound of the disclosure and a PI3K or AKT inhibitor can be greater than a median survival time of a second patient population that does not receive any cancer therapy. In some embodiments, a median survival time of a first patient population receiving combination therapy with a compound of the disclosure and a PI3K or AKT inhibitor can be greater than a median survival time of a second patient population that receives therapy with a compound of the disclosure alone. In some embodiments, a median survival time of a first patient population receiving combination therapy with a compound of the disclosure and a PI3K or AKT inhibitor can be greater than a median survival time of a second patient population that receives therapy with a PI3K or AKT inhibitor alone.

In some embodiments, a median survival time of a first patient population receiving combination therapy with a compound of the disclosure and a MDM2 inhibitor can be greater than a median survival time of a second patient population not receiving combination therapy by at least about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some embodiments, a median survival time of a first patient population receiving combination therapy with a compound of the disclosure and a MDM2 inhibitor can be greater than a median survival time of a second patient population not receiving combination therapy by at least about 50%. In some embodiments, a median survival time of a first patient population receiving combination therapy with a compound of the disclosure and a MDM2 inhibitor can be greater than a median survival time of a second patient population not receiving combination therapy by at least about 100%. In some embodiments, a median survival time of a first patient population receiving combination therapy with a compound of the disclosure and a MDM2 inhibitor can be greater than a median survival time of a second patient population not receiving combination therapy by at least about 150%. In some embodiments, a median survival time of a first patient population receiving combination therapy with a compound of the disclosure and a MDM2 inhibitor can be greater than a median survival time of a second patient population not receiving combination therapy by at least about 200%. In some embodiments, a median survival time of a first patient population receiving combination therapy with a compound of the disclosure and a PI3K or AKT inhibitor can be greater than a median survival time of a second patient population not receiving combination therapy by at least about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%. In some embodiments, a median survival time of a first patient population receiving combination therapy with a compound of the disclosure and a PI3K or AKT inhibitor can be greater than a median survival time of a second patient population not receiving combination therapy by at least about 50%. In some embodiments, a median survival time of a first patient population receiving combination therapy with a compound of the disclosure and a PI3K or AKT inhibitor can be greater than a median survival time of a second patient population not receiving combination therapy by at least about 100%. In some embodiments, a median survival time of a first patient population receiving combination therapy with a compound of the disclosure and a PI3K or AKT inhibitor can be greater than a median survival time of a second patient population not receiving combination therapy by at least about 150%. In some embodiments, a median survival time of a first patient population receiving combination therapy with a compound of the disclosure and a PI3K or AKT inhibitor can be greater than a median survival time of a second patient population not receiving combination therapy by at least about 200%.

### Methods of Treatment

Provided herein is a method of treating cancer in a subject in need thereof, the method comprising: (i) administering to the subject a therapeutically-effective amount of a compound, wherein the compound binds to a mutant p53 protein and reconforms the mutant p53 protein to a conformation of p53 that exhibits anti-cancer activity; and (ii) administering to the subject a therapeutically-effective amount of an anti-cancer agent that functions through a pathway other than p53-induced apoptosis.

Also provided herein is a method of treating cancer in a subject in need thereof, the method comprising: (i) administering to the subject a therapeutically-effective amount of a compound that increases anti-cancer activity of a mutant p53 protein in the subject; and (ii) administering to the subject a therapeutically-effective amount of an anti-cancer agent that functions through a MDM2 pathway. In some embodiments, the anti-cancer agent that functions through a MDM2 pathway is a MDM2 inhibitor. In some embodiments, the anti-cancer agent that functions through a MDM2 pathway binds a MDM2 protein. Also provided herein is a method of treating cancer in a subject in need thereof, the method comprising: (i) administering to the subject a therapeutically-effective amount of a compound that increases anti-cancer activity of a mutant p53 protein in the subject; and (ii) administering to the subject a therapeutically-effective amount of an anti-cancer agent that functions through a PI3K or AKT pathway. In some embodiments, the anti-cancer agent that functions through a MDM-2 pathway is a PI3K or AKT inhibitor. In some embodiments, the anti-cancer agent that functions through a PI3K or AKT pathway binds a PI3K or AKT protein.

In some embodiments, the compound binds to the mutant p53 protein and reconforms the mutant p53 protein to a conformation of p53 that exhibits anti-cancer activity.

Further provided herein is a method of treating cancer, the method comprising: (i) administering to a subject in need thereof a therapeutically-effective amount of a compound that binds to a mutant p53 protein and reconforms the mutant p53 protein to a conformation of p53 that exhibits anti-cancer activity; and (ii) administering to the subject a therapeutically-effective amount of an additional anti-cancer agent that functions through a pathway other than p53-induced apoptosis (e.g., a MDM2 inhibitor), wherein if in a controlled study of treatment of the cancer in a first patient population and a second patient population: (a) a first median survival time of the first patient population is determined, wherein the first patient population is treated with the therapeutically-effective amount of the compound that binds to a mutant p53 protein and reconforms the mutant p53 protein to a conformation of p53 that exhibits anti-cancer activity; and (b) a second median survival time of the second patient population is determined, wherein the second patient population is treated with the therapeutically-effective amount of the compound that binds to a mutant p53 protein and reconforms the mutant p53 protein to a conformation of p53 that exhibits anti-cancer activity and the therapeutically-effective amount of the additional therapeutic agent; then the second median survival time is at least about 50% greater than is the first median survival time.

In some embodiments, the second median survival time is at least about 100% greater than the first median survival time. In some embodiments, the second median survival time is at least about 200% greater than the first median survival time.

In some embodiments, the compound increases a stability of the mutant p53 protein. In some embodiments, the cancer expresses a mutant p53 protein. In some embodiments, the mutant p53 protein has a mutation at amino acid 220. In some embodiments, the mutant p53 protein is p53 Y220C. In some embodiments, the compound selectively binds the mutant p53 protein as compared to a wild type p53. In some embodiments, the cancer is ovarian cancer. In some embodiments, the cancer is breast cancer. In some embodiments, the cancer is lung cancer. In some embodiments, the subject is human.

In some embodiments, the administering of the compound is oral. In some embodiments, the administering of the compound is subcutaneous. In some embodiments, the administering of the compound is topical. In some embodiments, the therapeutically-effective amount of the compound is from about 1 mg/kg to about 500 mg/kg. In some embodiments, the therapeutically-effective amount of the compound is from about 100 mg to about 5000 mg. In some embodiments, the therapeutically-effective amount of the compound is from about 500 mg to about 2000 mg. In some embodiments, the therapeutically-effective amount of the compound is about 250 mg, about 500 mg, about 750 mg, about 1000 mg, about 1250 mg, about 1500 mg, about 1750 mg, about 2000 mg, about 2250 mg, or about 2500 mg. In some embodiments, the therapeutically-effective amount of the compound is about 150 mg. In some embodiments, the therapeutically-effective amount of the compound is about 300 mg. In some embodiments, the therapeutically-effective amount of the compound is about 500 mg. In some embodiments, the therapeutically-effective amount of the compound is about 600 mg. In some embodiments, the therapeutically-effective amount of the compound is about 1200 mg. In some embodiments, the therapeutically-effective amount of the compound is about 1500 mg. In some embodiments, the therapeutically-effective amount of the compound is about 2000 mg.

In some embodiments, the MDM2 inhibitor is a small molecule. In some embodiments, the MDM2 inhibitor is an antibody. In some embodiments, the MDM2 inhibitor binds MDM2 protein.

In some embodiments, the MDM2 inhibitor is

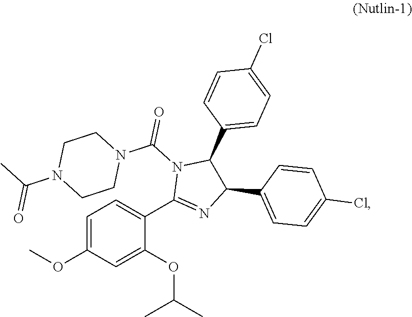 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1]

or a pharmaceutically-acceptable salt thereof.

In some embodiments, the MDM2 inhibitor is

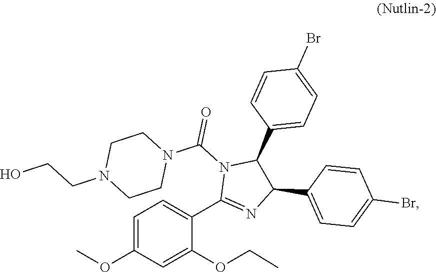 [SMILES: CCOc1cc(OC)ccc1C1=N[C@H](c2ccc(Br)cc2)[C@H](c2ccc(Br)cc2)N1C(=O)N1CCN(CCO)CC1]

or a pharmaceutically-acceptable salt thereof.

In some embodiments, the MDM2 inhibitor is

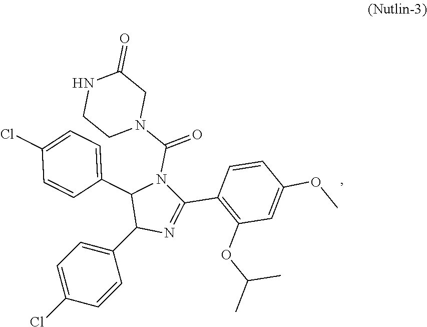 [SMILES: COc1ccc(C2=NC(c3ccc(Cl)cc3)C(c3ccc(Cl)cc3)N2C(=O)N2CCNC(=O)C2)c(OC(C)C)c1]

or a pharmaceutically-acceptable salt thereof.

In some embodiments, the MDM2 inhibitor is

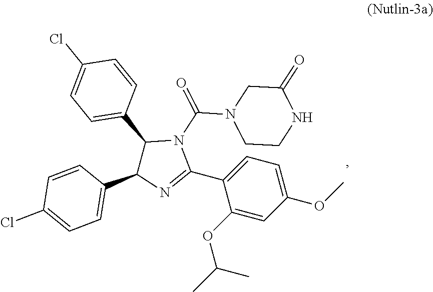 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

or a pharmaceutically-acceptable salt thereof.

In some embodiments, the MDM2 inhibitor is

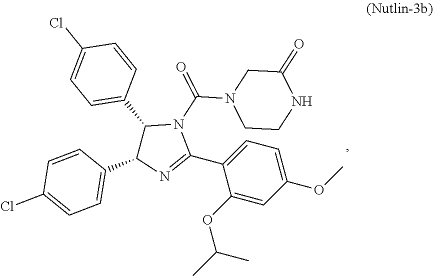 [SMILES: COc1ccc(C2=N[C@H](c3ccc(Cl)cc3)[C@H](c3ccc(Cl)cc3)N2C(=O)N2CCNC(=O)C2)c(OC(C)C)c1]

or a pharmaceutically-acceptable salt thereof.

In some embodiments, the MDM2 inhibitor is

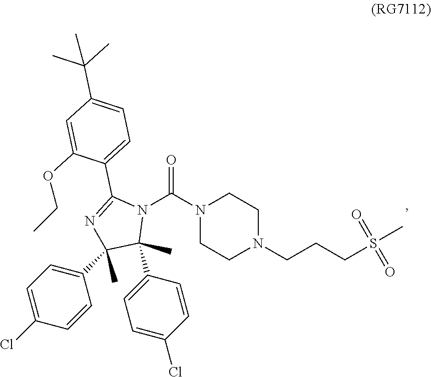 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

or a pharmaceutically-acceptable salt thereof.

In some embodiments, the MDM2 inhibitor is

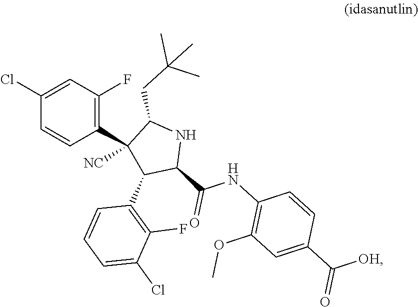 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

or a pharmaceutically-acceptable salt thereof.

In some embodiments, the MDM2 inhibitor is

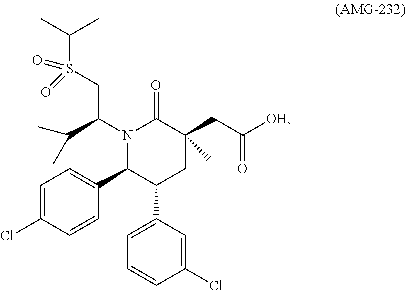 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically-acceptable salt thereof.

In some embodiments, the MDM2 inhibitor is

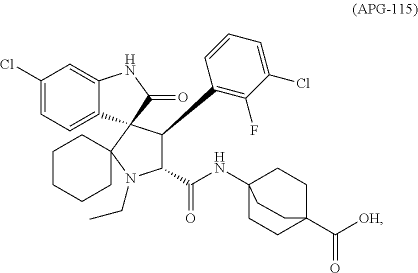 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically-acceptable salt thereof.

In some embodiments, the MDM2 inhibitor is

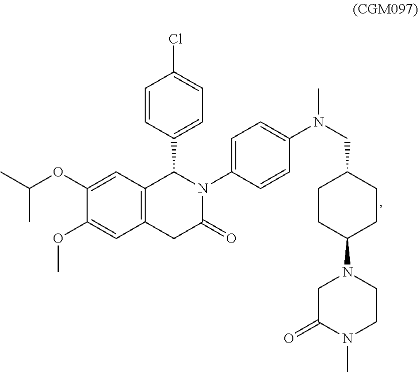 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

or a pharmaceutically acceptable salt thereof.

In some embodiments, the MDM2 inhibitor is

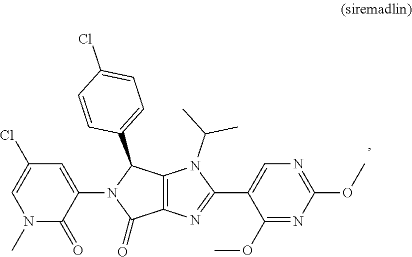 [SMILES: COc1ncc(-c2nc3c(n2C(C)C)[C@H](c2ccc(Cl)cc2)N(c2cc(Cl)cn(C)c2=O)C3=O)c(OC)n1]

or a pharmaceutically-acceptable salt thereof.

In some embodiments, the MDM2 inhibitor is

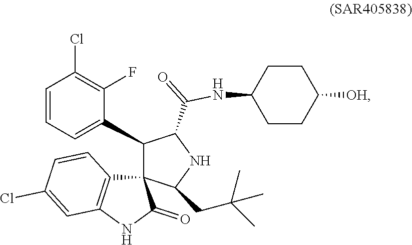 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

or a pharmaceutically-acceptable salt thereof.

In some embodiments, the MDM2 inhibitor is

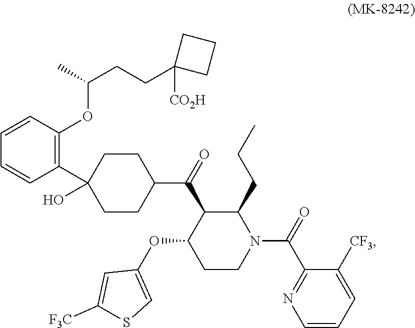 [SMILES: CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F]

or a pharmaceutically-acceptable salt thereof.

In some embodiments, the MDM2 inhibitor is

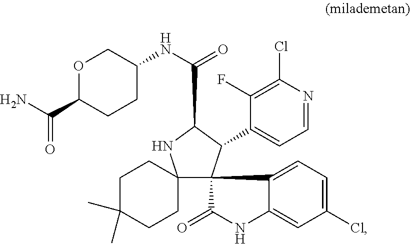 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

or a pharmaceutically-acceptable salt thereof.

In some embodiments, the MDM2 inhibitor is

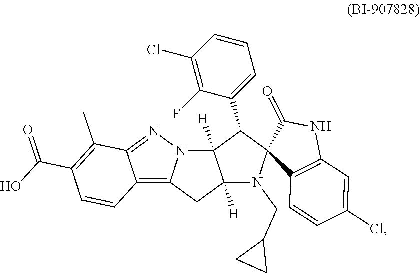 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

or a pharmaceutically-acceptable salt thereof.

In some embodiments, the MDM2 inhibitor is

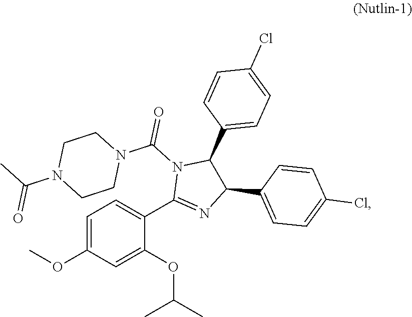 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1]

or a pharmaceutically-acceptable salt thereof, and the compound that increases anti-tumor activity of the mutant p53 protein is

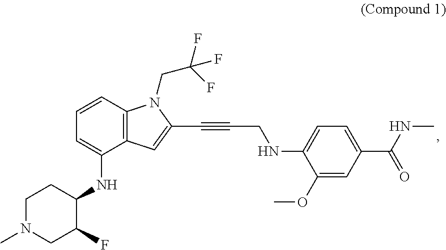

or a pharmaceutically-acceptable salt thereof.

In some embodiments, the MDM2 inhibitor is

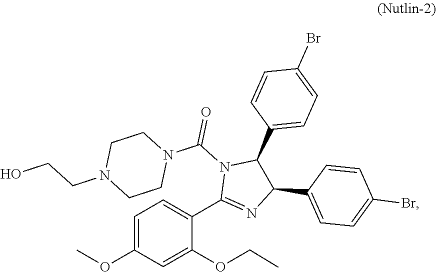 [SMILES: CCOc1cc(OC)ccc1C1=N[C@H](c2ccc(Br)cc2)[C@H](c2ccc(Br)cc2)N1C(=O)N1CCN(CCO)CC1]

or a pharmaceutically-acceptable salt thereof, and the compound that increases anti-tumor activity of the mutant p53 protein is

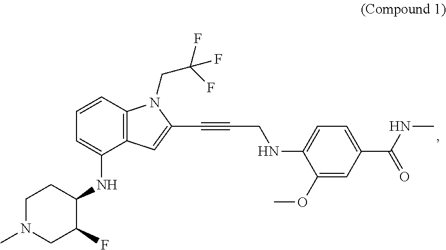

or a pharmaceutically-acceptable salt thereof.

In some embodiments, the MDM2 inhibitor is

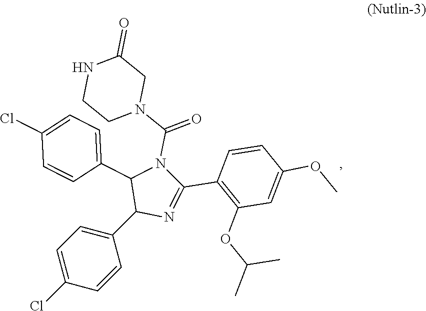 [SMILES: COc1ccc(C2=NC(c3ccc(Cl)cc3)C(c3ccc(Cl)cc3)N2C(=O)N2CCNC(=O)C2)c(OC(C)C)c1]

or a pharmaceutically-acceptable salt thereof, and the compound that increases anti-tumor activity of the mutant p53 protein is

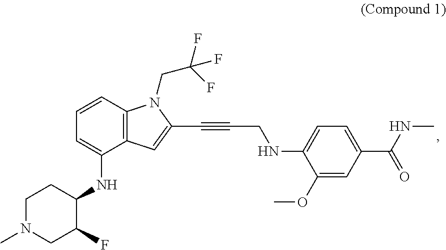

or a pharmaceutically-acceptable salt thereof.

In some embodiments, the MDM2 inhibitor is

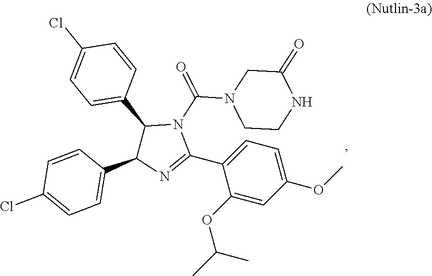 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

or a pharmaceutically-acceptable salt thereof, and the compound that increases anti-tumor activity of the mutant p53 protein is

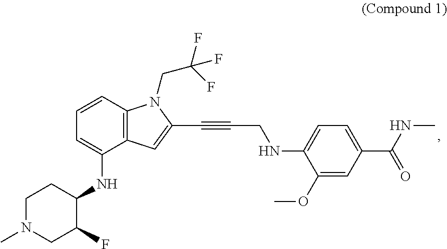

or a pharmaceutically-acceptable salt thereof.

In some embodiments, the MDM2 inhibitor is

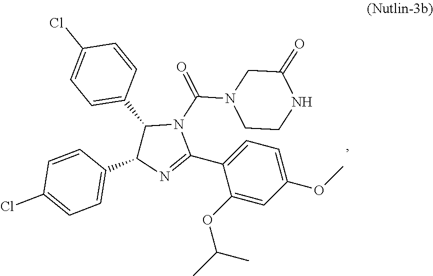 [SMILES: COc1ccc(C2=N[C@H](c3ccc(Cl)cc3)[C@H](c3ccc(Cl)cc3)N2C(=O)N2CCNC(=O)C2)c(OC(C)C)c1]

or a pharmaceutically-acceptable salt thereof, and the compound that increases anti-tumor activity of the mutant p53 protein is

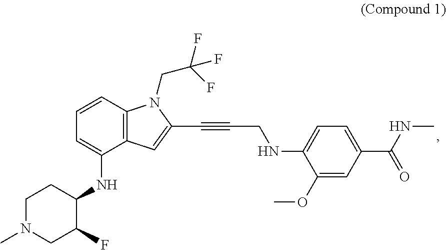

or a pharmaceutically-acceptable salt thereof.

In some embodiments, the MDM2 inhibitor is

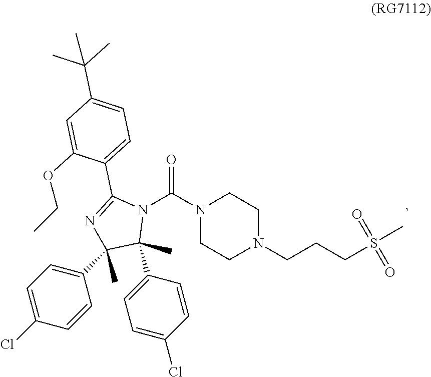 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

or a pharmaceutically-acceptable salt thereof, and the compound that increases anti-tumor activity of the mutant p53 protein is

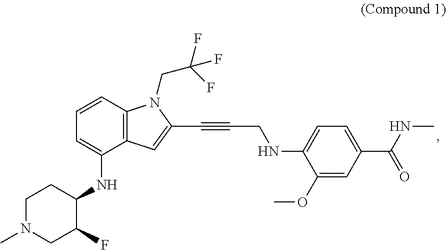

or a pharmaceutically-acceptable salt thereof.

In some embodiments, the MDM2 inhibitor is

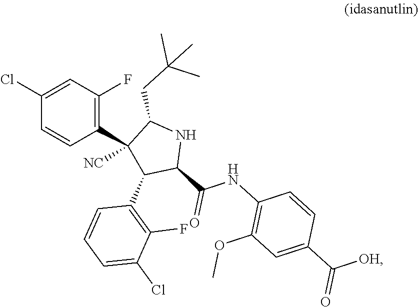 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

a pharmaceutically-acceptable salt thereof, and the compound that increases anti-tumor activity of the mutant p53 protein is

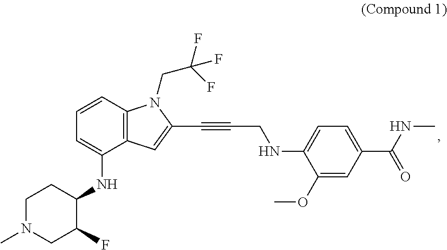

or a pharmaceutically-acceptable salt thereof.

In some embodiments, the MDM2 inhibitor is

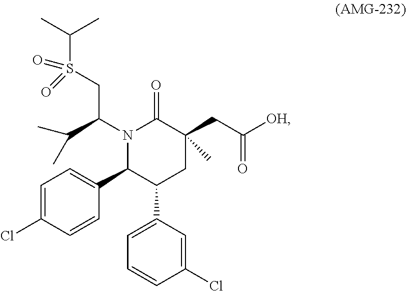 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically-acceptable salt thereof, and the compound that increases anti-tumor activity of the mutant p53 protein is

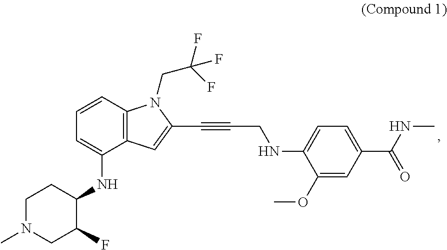

or a pharmaceutically-acceptable salt thereof.

In some embodiments, the MDM2 inhibitor is

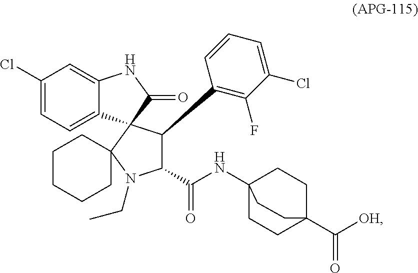 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically-acceptable salt thereof, and the compound that increases anti-tumor activity of the mutant p53 protein is

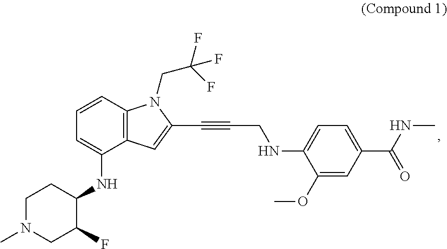

or a pharmaceutically-acceptable salt thereof.

In some embodiments, the MDM2 inhibitor is

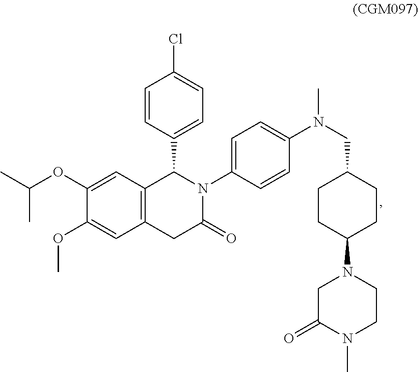 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

or a pharmaceutically acceptable salt thereof, and the compound that increases anti-tumor activity of the mutant p53 protein is

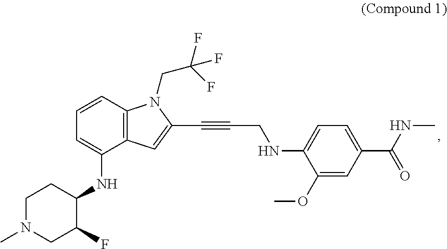

or a pharmaceutically-acceptable salt thereof.

In some embodiments, the MDM2 inhibitor is

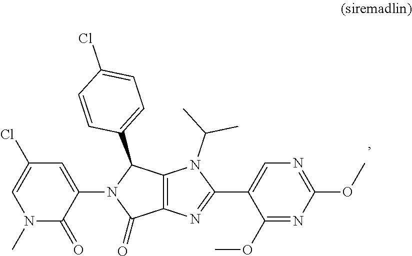 [SMILES: COc1ncc(-c2nc3c(n2C(C)C)[C@H](c2ccc(Cl)cc2)N(c2cc(Cl)cn(C)c2=O)C3=O)c(OC)n1]

or a pharmaceutically-acceptable salt thereof, and the compound that increases anti-tumor activity of the mutant p53 protein is

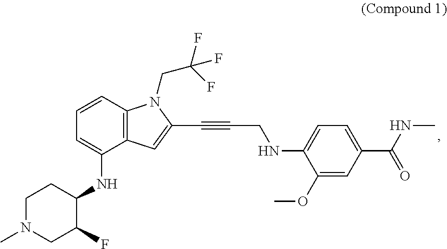

or a pharmaceutically-acceptable salt thereof.

In some embodiments, the MDM2 inhibitor is

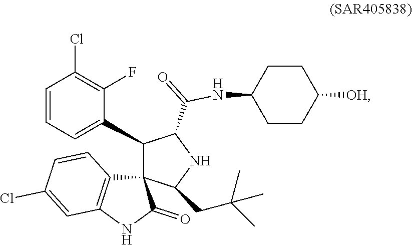 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

or a pharmaceutically-acceptable salt thereof, and the compound that increases anti-tumor activity of the mutant p53 protein is

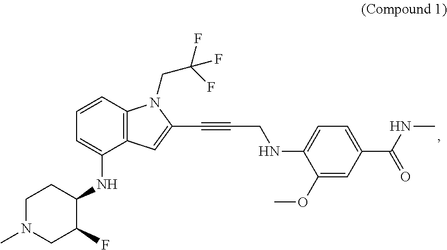

or a pharmaceutically-acceptable salt thereof.

In some embodiments, the MDM2 inhibitor is

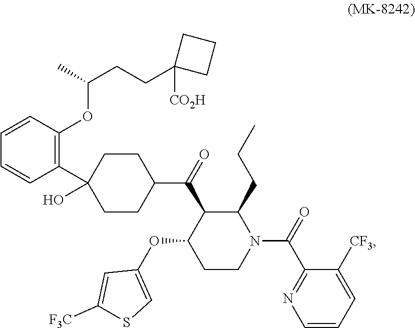 [SMILES: CCC[C@@H]1[C@H](C(=O)C2CCC(O)(c3ccccc3O[C@H](C)CCC3(C(=O)O)CCC3)CC2)[C@@H](Oc2csc(C(F)(F)F)c2)CCN1C(=O)c1ncccc1C(F)(F)F]

or a pharmaceutically-acceptable salt thereof, and the compound that increases anti-tumor activity of the mutant p53 protein is

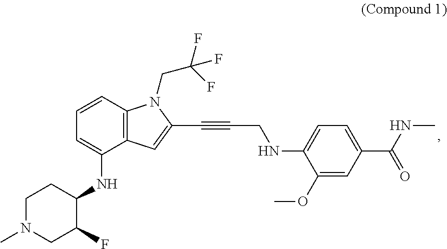

or a pharmaceutically-acceptable salt thereof.

In some embodiments, the MDM2 inhibitor is

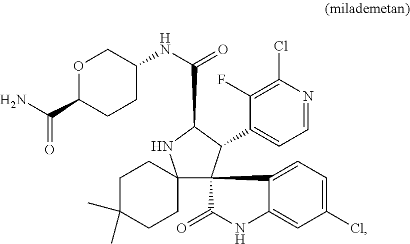 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

or a pharmaceutically-acceptable salt thereof, and the compound that increases anti-tumor activity of the mutant p53 protein is

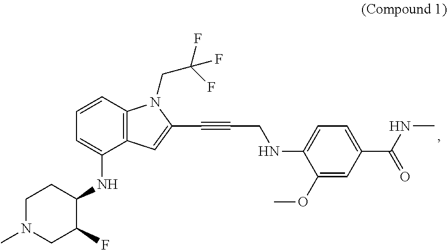

or a pharmaceutically-acceptable salt thereof.

In some embodiments, the MDM2 inhibitor is

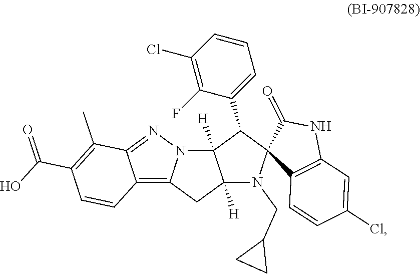 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

or a pharmaceutically-acceptable salt thereof, and the compound that increases anti-tumor activity of the mutant p53 protein is

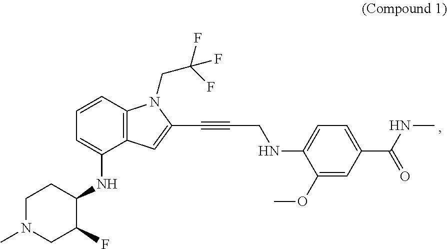

or a pharmaceutically-acceptable salt thereof.

In some embodiments, the PI3K inhibitor is alpelisib. In some embodiments, the PI3K inhibitor is inavolisib. In some embodiments, the PI3K inhibitor is serabelisib. In some embodiments, the PI3K inhibitor is GSK263. In some embodiments, the PI3K inhibitor is eganelisib. In some embodiments, the PI3K inhibitor is idelalisib. In some embodiments, the PI3K inhibitor is duvelisib. In some embodiments, the PI3K inhibitor is ipatasertib. In some embodiments, the PI3K inhibitor is capivasertib.

In some embodiments, the AKT inhibitor is MK2206. In some embodiments, the AKT inhibitor is ipatasertib.

In some embodiments, the administering of the anti-cancer agent is oral. In some embodiments, the administering of the anti-cancer agent is subcutaneous. In some embodiments, the administering of the anti-cancer agent is topical. In some embodiments, the therapeutically-effective amount of the anti-cancer agent is from about 5 mg/kg to about 100 mg/kg. In some embodiments, the therapeutically-effective amount of the anti-cancer agent is from about 10 mg to about 1500 mg. In some embodiments, the therapeutically-effective amount of the anti-cancer agent is about 1000 mg.

Pharmacokinetic and pharmacodynamic data can be obtained by various experimental techniques. Appropriate pharmacokinetic and pharmacodynamic profile components describing a particular composition can vary due to variations in drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean is determined, for example, by calculating the average of all subject's measurements for each parameter measured. A dose can be modulated to achieve a desired pharmacokinetic or pharmacodynamics profile, such as a desired or effective blood profile, as described herein.

The pharmacodynamic parameters can be any parameters suitable for describing compositions of the invention. For example, the pharmacodynamic profile can be obtained at a time after dosing of, for example, about zero minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about zero hours, about 0.5 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, or about 24 hours.

The pharmacokinetic parameters can be any parameters suitable for describing a compound. The Cₘₐₓ can be, for example, not less than about 1 ng/mL; not less than about 5 ng/mL; not less than about 10 ng/mL; not less than about 15 ng/mL; not less than about 20 ng/mL; not less than about 25 ng/mL; not less than about 50 ng/mL; not less than about 75 ng/mL; not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; or any other Cₘₐₓ appropriate for describing a pharmacokinetic profile of a compound described herein. The Cₘₐₓ can be, for example, about 1 ng/mL to about 5,000 ng/mL; about 1 ng/mL to about 4,500 ng/mL; about 1 ng/mL to about 4,000 ng/mL; about 1 ng/mL to about 3,500 ng/mL; about 1 ng/mL to about 3,000 ng/mL; about 1 ng/mL to about 2,500 ng/mL; about 1 ng/mL to about 2,000 ng/mL; about 1 ng/mL to about 1,500 ng/mL; about 1 ng/mL to about 1,000 ng/mL; about 1 ng/mL to about 900 ng/mL; about 1 ng/mL to about 800 ng/mL; about 1 ng/mL to about 700 ng/mL; about 1 ng/mL to about 600 ng/mL; about 1 ng/mL to about 500 ng/mL; about 1 ng/mL to about 450 ng/mL; about 1 ng/mL to about 400 ng/mL; about 1 ng/mL to about 350 ng/mL; about 1 ng/mL to about 300 ng/mL; about 1 ng/mL to about 250 ng/mL; about 1 ng/mL to about 200 ng/mL; about 1 ng/mL to about 150 ng/mL; about 1 ng/mL to about 125 ng/mL; about 1 ng/mL to about 100 ng/mL; about 1 ng/mL to about 90 ng/mL; about 1 ng/mL to about 80 ng/mL; about 1 ng/mL to about 70 ng/mL; about 1 ng/mL to about 60 ng/mL; about 1 ng/mL to about 50 ng/mL; about 1 ng/mL to about 40 ng/mL; about 1 ng/mL to about 30 ng/mL; about 1 ng/mL to about 20 ng/mL; about 1 ng/mL to about 10 ng/mL; about 1 ng/mL to about 5 ng/mL; about 10 ng/mL to about 4,000 ng/mL; about 10 ng/mL to about 3,000 ng/mL; about 10 ng/mL to about 2,000 ng/mL; about 10 ng/mL to about 1,500 ng/mL; about 10 ng/mL to about 1,000 ng/mL; about 10 ng/mL to about 900 ng/mL; about 10 ng/mL to about 800 ng/mL; about 10 ng/mL to about 700 ng/mL; about 10 ng/mL to about 600 ng/mL; about 10 ng/mL to about 500 ng/mL; about 10 ng/mL to about 400 ng/mL; about 10 ng/mL to about 300 ng/mL; about 10 ng/mL to about 200 ng/mL; about 10 ng/mL to about 100 ng/mL; about 10 ng/mL to about 50 ng/mL; about 25 ng/mL to about 500 ng/mL; about 25 ng/mL to about 100 ng/mL; about 50 ng/mL to about 500 ng/mL; about 50 ng/mL to about 100 ng/mL; about 100 ng/mL to about 500 ng/mL; about 100 ng/mL to about 400 ng/mL; about 100 ng/mL to about 300 ng/mL; or about 100 ng/mL to about 200 ng/mL.

The Tₘₐₓ of a compound described herein can be, for example, not greater than about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, or any other Tₘₐₓ appropriate for describing a pharmacokinetic profile of a compound described herein. The Tₘₐₓ can be, for example, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; or about 23.5 hours to about 24 hours. In some embodiments, the Tₘₐₓ of a compound of the disclosure is about 2 hours. In some embodiments, the Tₘₐₓ of a compound of the disclosure is about 4 hours. In some embodiments, the Tₘₐₓ of a compound of the disclosure is about 6 hours. In some embodiments, the Tₘₐₓ of a compound of the disclosure is about 8 hours.

The AUC_{(0-inf)} or AUC₍ₗₐₛₜ₎ of a compound described herein can be, for example, not less than about 1 ng·hr/mL, not less than about 5 ng·hr/mL, not less than about 10 ng·hr/mL, not less than about 20 ng·hr/mL, not less than about 30 ng·hr/mL, not less than about 40 ng·hr/mL, not less than about 50 ng·hr/mL, not less than about 100 ng·hr/mL, not less than about 150 ng·hr/mL, not less than about 200 ng·hr/mL, not less than about 250 ng·hr/mL, not less than about 300 ng·hr/mL, not less than about 350 ng·hr/mL, not less than about 400 ng·hr/mL, not less than about 450 ng·hr/mL, not less than about 500 ng·hr/mL, not less than about 600 ng·hr/mL, not less than about 700 ng·hr/mL, not less than about 800 ng·hr/mL, not less than about 900 ng·hr/mL, not less than about 1000 ng·hr/mL, not less than about 1250 ng·hr/mL, not less than about 1500 ng·hr/mL, not less than about 1750 ng·hr/mL, not less than about 2000 ng·hr/mL, not less than about 2500 ng·hr/mL, not less than about 3000 ng·hr/mL, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, not less than about 10,000 ng·hr/mL, or any other AUC_{(0-inf)} or AUC₍ₗₐₛₜ₎ appropriate for describing a pharmacokinetic profile of a compound described herein. In some embodiments, the AUC_{(0-inf)} or AUC₍ₗₐₛₜ₎ of a compound described herein can be, for example, not less than about 10,000 ng·hr/mL, not less than about 11,000 ng·hr/mL, not less than about 12,000 ng·hr/mL, not less than about 13,000 ng·hr/mL, not less than about 14,000 ng·hr/mL, not less than about 15,000 ng·hr/mL, not less than about 16,000 ng·hr/mL, not less than about 17,000 ng·hr/mL, not less than about 18,000 ng·hr/mL, not less than about 19,000 ng·hr/mL, not less than about 20,000 ng·hr/mL, not less than about 21,000 ng·hr/mL, not less than about 22,000 ng·hr/mL, not less than about 23,000 ng·hr/mL, not less than about 24,000 ng·hr/mL, or not less than about 25,000 ng·hr/mL.

The AUC_{(0-inf)} or AUC₍ₗₐₛₜ₎ of a compound can be, for example, about 1 ng·hr/mL to about 10,000 ng·hr/mL; about 1 ng·hr/mL to about 10 ng·hr/mL; about 10 ng·hr/mL to about 25 ng·hr/mL; about 25 ng·hr/mL to about 50 ng·hr/mL; about 50 ng·hr/mL to about 100 ng·hr/mL; about 100 ng·hr/mL to about 200 ng·hr/mL; about 200 ng·hr/mL to about 300 ng·hr/mL; about 300 ng·hr/mL to about 400 ng·hr/mL; about 400 ng·hr/mL to about 500 ng·hr/mL; about 500 ng·hr/mL to about 600 ng·hr/mL; about 600 ng·hr/mL to about 700 ng·hr/mL; about 700 ng·hr/mL to about 800 ng·hr/mL; about 800 ng·hr/mL to about 900 ng·hr/mL; about 900 ng·hr/mL to about 1,000 ng·hr/mL; about 1,000 ng·hr/mL to about 1,250 ng·hr/mL; about 1,250 ng·hr/mL to about 1,500 ng·hr/mL; about 1,500 ng·hr/mL to about 1,750 ng·hr/mL; about 1,750 ng·hr/mL to about 2,000 ng·hr/mL; about 2,000 ng·hr/mL to about 2,500 ng·hr/mL; about 2,500 ng·hr/mL to about 3,000 ng·hr/mL; about 3,000 ng·hr/mL to about 3,500 ng·hr/mL; about 3,500 ng·hr/mL to about 4,000 ng·hr/mL; about 4,000 ng·hr/mL to about 4,500 ng·hr/mL; about 4,500 ng·hr/mL to about 5,000 ng·hr/mL; about 5,000 ng·hr/mL to about 5,500 ng·hr/mL; about 5,500 ng·hr/mL to about 6,000 ng·hr/mL; about 6,000 ng·hr/mL to about 6,500 ng·hr/mL; about 6,500 ng·hr/mL to about 7,000 ng·hr/mL; about 7,000 ng·hr/mL to about 7,500 ng·hr/mL; about 7,500 ng·hr/mL to about 8,000 ng·hr/mL; about 8,000 ng·hr/mL to about 8,500 ng·hr/mL; about 8,500 ng·hr/mL to about 9,000 ng·hr/mL; about 9,000 ng·hr/mL to about 9,500 ng·hr/mL; or about 9,500 ng·hr/mL to about 10,000 ng·hr/mL. In some embodiments, the AUC_{(0-inf)} or AUC₍ₗₐₛₜ₎ of a compound described herein can be, for example, about 10,000 ng·hr/mL, about 11,000 ng·hr/mL, about 12,000 ng·hr/mL, about 13,000 ng·hr/mL, about 14,000 ng·hr/mL, about 15,000 ng·hr/mL, about 16,000 ng·hr/mL, about 17,000 ng·hr/mL, about 18,000 ng·hr/mL, about 19,000 ng·hr/mL, about 20,000 ng·hr/mL, about 21,000 ng·hr/mL, about 22,000 ng·hr/mL, about 23,000 ng·hr/mL, about 24,000 ng·hr/mL, or about 25,000 ng·hr/mL.

The plasma concentration of a compound described herein can be, for example, not less than about 1 ng/mL, not less than about 5 ng/mL, not less than about 10 ng/mL, not less than about 15 ng/mL, not less than about 20 ng/mL, not less than about 25 ng/mL, not less than about 50 ng/mL, not less than about 75 ng/mL, not less than about 100 ng/mL, not less than about 150 ng/mL, not less than about 200 ng/mL, not less than about 300 ng/mL, not less than about 400 ng/mL, not less than about 500 ng/mL, not less than about 600 ng/mL, not less than about 700 ng/mL, not less than about 800 ng/mL, not less than about 900 ng/mL, not less than about 1000 ng/mL, not less than about 1200 ng/mL, or any other plasma concentration of a compound described herein. The plasma concentration can be, for example, about 1 ng/mL to about 2,000 ng/mL; about 1 ng/mL to about 5 ng/mL; about 5 ng/mL to about 10 ng/mL; about 10 ng/mL to about 25 ng/mL; about 25 ng/mL to about 50 ng/mL; about 50 ng/mL to about 75 ng/mL; about 75 ng/mL to about 100 ng/mL; about 100 ng/mL to about 150 ng/mL; about 150 ng/mL to about 200 ng/mL; about 200 ng/mL to about 250 ng/mL; about 250 ng/mL to about 300 ng/mL; about 300 ng/mL to about 350 ng/mL; about 350 ng/mL to about 400 ng/mL; about 400 ng/mL to about 450 ng/mL; about 450 ng/mL to about 500 ng/mL; about 500 ng/mL to about 600 ng/mL; about 600 ng/mL to about 700 ng/mL; about 700 ng/mL to about 800 ng/mL; about 800 ng/mL to about 900 ng/mL; about 900 ng/mL to about 1,000 ng/mL; about 1,000 ng/mL to about 1,100 ng/mL; about 1,100 ng/mL to about 1,200 ng/mL; about 1,200 ng/mL to about 1,300 ng/mL; about 1,300 ng/mL to about 1,400 ng/mL; about 1,400 ng/mL to about 1,500 ng/mL; about 1,500 ng/mL to about 1,600 ng/mL; about 1,600 ng/mL to about 1,700 ng/mL; about 1,700 ng/mL to about 1,800 ng/mL; about 1,800 ng/mL to about 1,900 ng/mL; or about 1,900 ng/mL to about 2,000 ng/mL.

In some embodiments, the plasma concentration can be about 2,500 ng/mL, about 3,000 ng/mL, about 3,500 ng/mL, about 4,000 ng/mL, about 4,500 ng/mL, about 5,000 ng/mL, about 5,500 ng/mL, about 6,000 ng/mL, about 6,500 ng/mL, about 7,000 ng/mL, about 7,500 ng/mL, about 8,000 ng/mL, about 8,500 ng/mL, about 9,000 ng/mL, about 9,500 ng/mL, or about 10,000 ng/mL. In some embodiments, the plasma concentration can be about 10,000 ng/mL, about 15,000 ng/mL, about 20,000 ng/mL, about 25,000 ng/mL, about 30,000 ng/mL, about 35,000 ng/mL, about 40,000 ng/mL, about 45,000 ng/mL, about 50,000 ng/mL, about 55,000 ng/mL, about 60,000 ng/mL, about 65,000 ng/mL, about 70,000 ng/mL, or about 75,000 ng/mL.

The pharmacodynamic parameters can be any parameters suitable for describing compositions of the disclosure. For example, the pharmacodynamic profile can exhibit decreases in viability phenotype for the tumor cells or tumor size reduction in tumor cell lines or xenograft studies, for example, about 24 hours, about 48 hours, about 72 hours, or 1 week.

### Numbered Embodiments

Embodiment 1. A method of treating a solid tumor in a subject in need thereof, the method comprising:
(i) administering to the subject a therapeutically-effective amount of a compound that increases anti-tumor activity of a mutant p53 protein in the subject; and
(ii) administering to the subject a therapeutically-effective amount of an anti-cancer agent that functions through a MDM2 pathway.

Embodiment 2. The method of embodiment 1, wherein the compound binds to the mutant p53 protein and reconforms the mutant p53 protein to a conformation of p53 that exhibits anti-tumor activity.

Embodiment 3. The method of embodiment 1 or embodiment 2, wherein the compound increases a stability of the mutant p53 protein.

Embodiment 4. The method of any one of embodiments 1-3, wherein the solid tumor expresses the mutant p53 protein.

Embodiment 5. The method of any one of embodiments 1-4, wherein the mutant p53 protein has a mutation at amino acid 220.

Embodiment 6. The method of any one of embodiments 1-4, wherein the mutant p53 protein is p53 Y220C.

Embodiment 7. The method of any one of embodiments 1-5, wherein the compound selectively binds the mutant p53 protein as compared to a wild type p53.

Embodiment 8. The method of any one of embodiments 1-6, wherein the solid tumor is selected from small cell lung cancer, pancreatic cancer, prostate cancer, breast cancer, endometrial cancer, and ovarian cancer.

Embodiment 9. The method of any one of embodiments 1-7, wherein the therapeutically-effective amount of the compound is from about 500 mg to about 3000 mg.

Embodiment 10. The method of any one of embodiments 1-8, wherein the subject is human.

Embodiment 11. The method of any one of embodiments 1-9, wherein the anti-cancer agent that functions through the MDM2 pathway is an MDM2 inhibitor.

Embodiment 12. The method of embodiment 11, wherein the MDM2 inhibitor is a small molecule.

Embodiment 13. The method of embodiment 11 or embodiment 12, wherein the MDM2 inhibitor is a compound, or a pharmaceutically-acceptable salt thereof, wherein the compound is selected from:

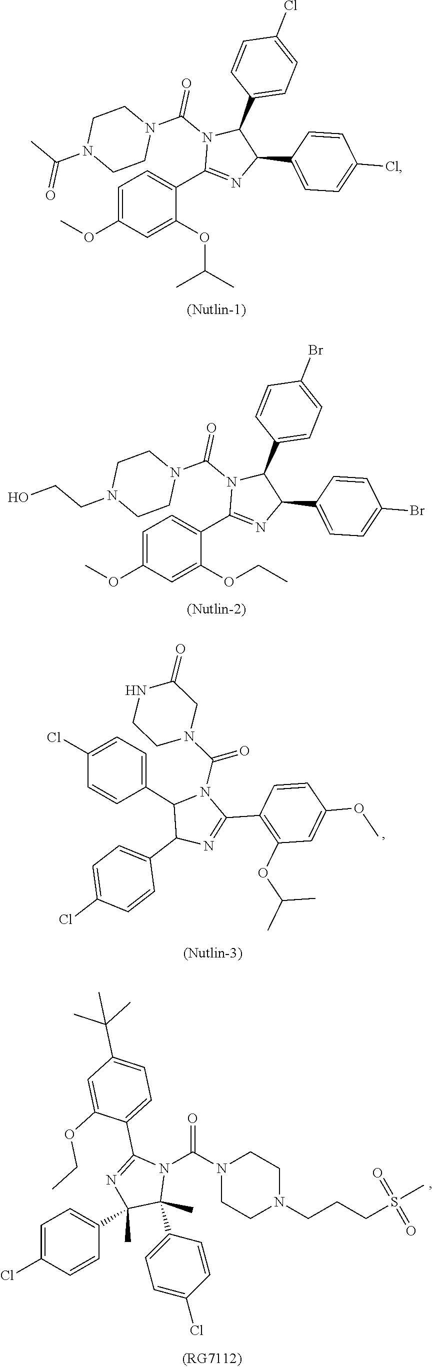 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1 ; CCOc1cc(OC)ccc1C1=N[C@H](c2ccc(Br)cc2)[C@H](c2ccc(Br)cc2)N1C(=O)N1CCN(CCO)CC1 ; COc1ccc(C2=NC(c3ccc(Cl)cc3)C(c3ccc(Cl)cc3)N2C(=O)N2CCNC(=O)C2)c(OC(C)C)c1 ; CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

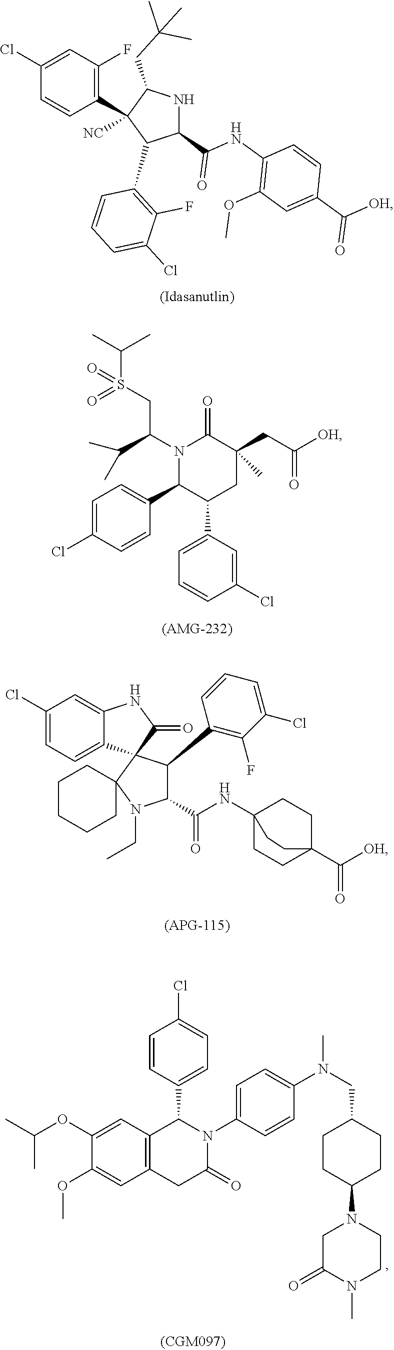

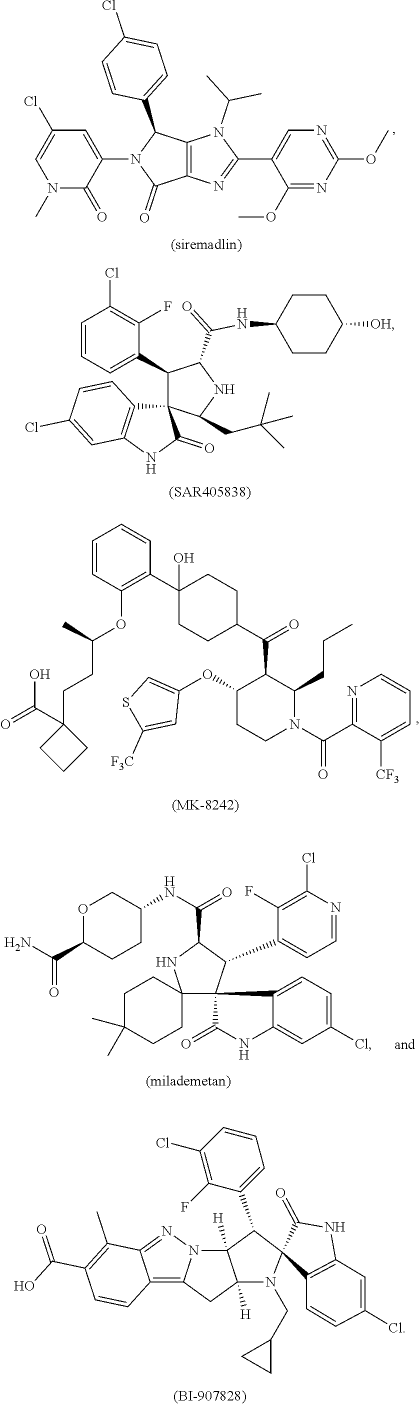

Embodiment 14. The method of any one of embodiments 11-13, wherein the therapeutically-effective amount of the MDM2 inhibitor is from about 5 mg/kg to about 500 mg/kg.

Embodiment 15. The method of any one of embodiments 1-14, wherein the compound that increases anti-tumor activity of the mutant p53 protein is of the formula:

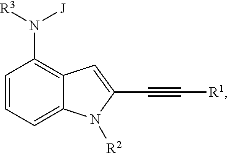

or a pharmaceutically-acceptable salt thereof,

wherein:
J is a cyclic group that is substituted or unsubstituted;
R¹ is alkyl or alkenyl, each of which is unsubstituted or substituted, or —C(O)R¹⁶, —C(O)OR¹⁶, or —C(O)NR¹⁶R¹⁷;
R¹⁶ and R¹⁷ are each independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², OC(O)R²¹, hydrogen, or halogen;
each R²¹ and R²² is independently alkyl, alkenyl, alkynyl, aryl, heteroaryl, or heterocyclyl, each of which is independently substituted or unsubstituted, or hydrogen;
R² is substituted or unsubstituted alkyl; and
R³ is H.

Embodiment 16. The method of embodiment 15, wherein J is aryl, heteroaryl, or heterocyclyl, each of which is substituted or unsubstituted.

Embodiment 17. The method of any embodiment 15 or embodiment 16, wherein J is unsubstituted or substituted heterocyclyl.

Embodiment 18. The method of any one of embodiments 15-17, wherein R² is substituted ethyl.

Embodiment 19. The method of any one of embodiments 15-17, wherein R² is trifluoroethyl.

Embodiment 20. The method of any one of embodiments 15-19, wherein R¹ is alkyl substituted with NR¹⁶R¹⁷; and each R¹⁶ and R¹⁷ is independently alkyl, alkenyl, aryl, heteroaryl, heterocyclyl, each of which is independently substituted or unsubstituted; or hydrogen.

Embodiment 21. The method of any one of embodiments 15-17, wherein the compound that increases anti-tumor activity of the mutant p53 protein is of the formula:

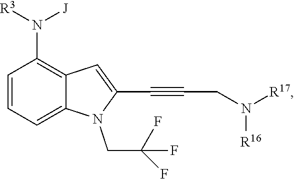

or a pharmaceutically-acceptable salt thereof.

Embodiment 22. The method of any one of embodiments 15-21, wherein R³ is H; J is piperidinyl, piperazinyl, tetahydropyranyl, morpholinyl, or pyrrolidinyl, each of which is independently substituted or unsubstituted; and each of R¹⁶ and R¹⁷ is independently alkyl, alkenyl, aryl, heteroaryl, heterocyclyl, each of which is independently substituted or unsubstituted, or H.

Embodiment 23. The method of any one of embodiments 15-22, wherein R¹⁶ is hydrogen or alkyl.

Embodiment 24. The method of any one of embodiments 15-23, wherein R¹⁷ is aryl, heteroaryl, or heterocyclyl, each of which is independently substituted or unsubstituted.

Embodiment 25. The method of any one of embodiments 15-24, wherein R¹⁷ is phenyl substituted with a sulfoxide group, sulfonyl group, carboxyl group, amide group, amino group, alkyl, alkoxy, hydroxy, or heterocyclyl, each of which is independently substituted or unsubstituted, or halo or cyano.

Embodiment 26. The method of any one of embodiments 1-25, wherein the compound that increases anti-tumor activity of the mutant p53 protein is

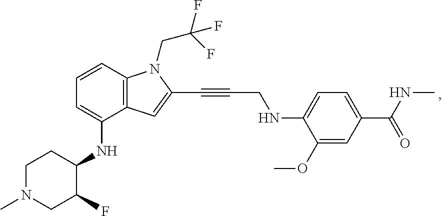

or a pharmaceutically-acceptable salt thereof.

Embodiment 27. A combination of a compound that increases anti-tumor activity of a mutant p53 protein in the subject; and an MDM2 inhibitor.

Embodiment 28. The combination of embodiment 27, wherein the compound that increases anti-tumor activity of the mutant p53 protein is contained in a first unit dosage form, and the MDM2 inhibitor is contained in a second unit dosage form.

Embodiment 29. The combination of embodiment 27 or embodiment 28, wherein the mutant p53 protein is p53 Y220C.

Embodiment 30. The combination of any one of embodiments 27-29, wherein the compound is of the formula:

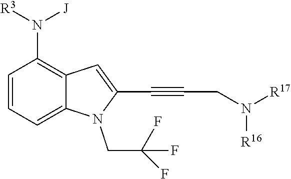

wherein R³ is H; J is piperidinyl, piperazinyl, tetahydropyranyl, morpholinyl, or pyrrolidinyl, each of which is independently substituted or unsubstituted; and each R¹⁶ and R¹⁷ is independently alkyl, alkenyl, aryl, heteroaryl, or heterocyclyl, each of which is independently substituted or unsubstituted, or H.

Embodiment 31. The combination of embodiment 30, wherein each R¹⁶ and R¹⁷ is independently alkyl, alkenyl, aryl, heteroaryl, heterocyclyl, each of which is independently substituted or unsubstituted; or hydrogen.

Embodiment 32. The combination of embodiment 30 or embodiment 31, wherein R¹⁶ is hydrogen or alkyl.

Embodiment 33. The combination of any one of embodiments 30-32, wherein R¹⁷ is aryl, heteroaryl, or heterocyclyl, each of which is independently substituted or unsubstituted.

Embodiment 34. The combination of any one of embodiments 30-33, wherein R¹⁷ is substituted aryl.

Embodiment 35. The combination of any one of embodiments 30-34, wherein R¹⁷ is phenyl substituted with a sulfoxide group, sulfonyl group, carboxyl group, amide group, amino group, alkyl, alkoxy, hydroxy, or heterocyclyl, each of which is independently substituted or unsubstituted, or halo or cyano.

Embodiment 36. The combination of any one of embodiments 27-35, wherein the MDM2 inhibitor is selected from:

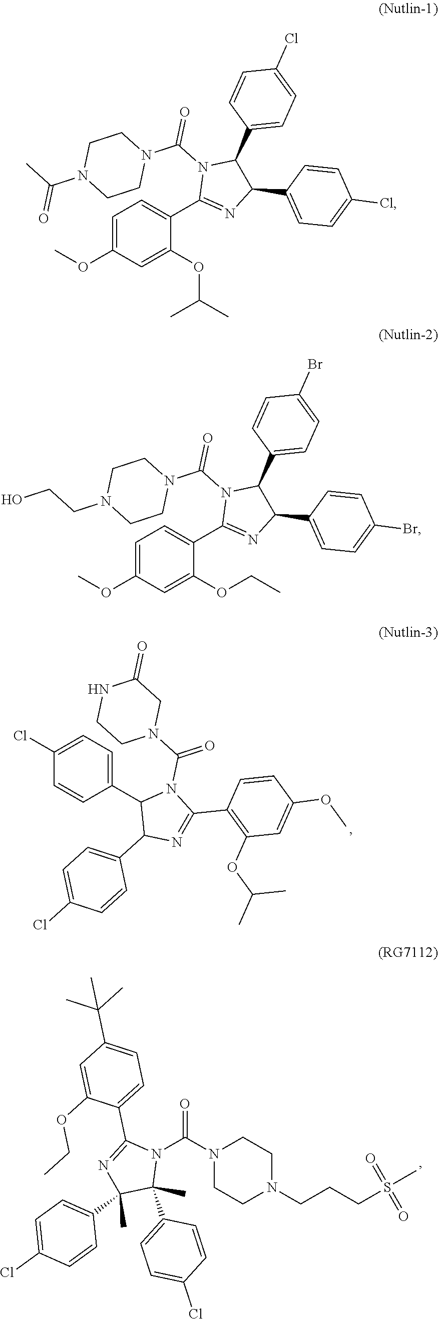 [SMILES: COc1ccc(C2=N[C@H](c3ccc(Cl)cc3)[C@H](c3ccc(Cl)cc3)N2C(=O)N2CCN(C(C)=O)CC2)c(OC(C)C)c1 ; CCOc1cc(OC)ccc1C1=N[C@H](c2ccc(Br)cc2)[C@H](c2ccc(Br)cc2)N1C(=O)N1CCN(CCO)CC1 ; COc1ccc(C2=NC(c3ccc(Cl)cc3)C(c3ccc(Cl)cc3)N2C(=O)N2CCNC(=O)C2)c(OC(C)C)c1 ; CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

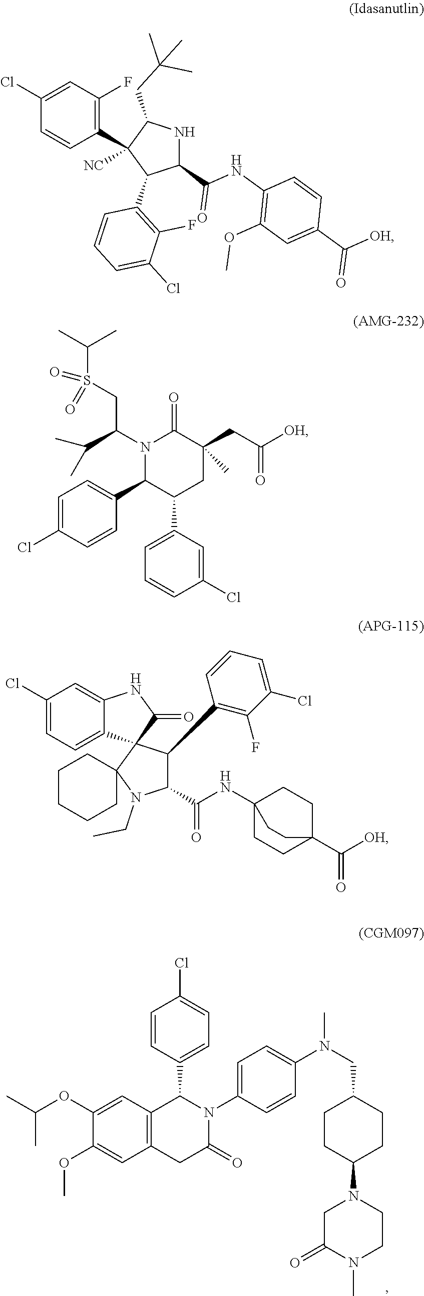 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F ; CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

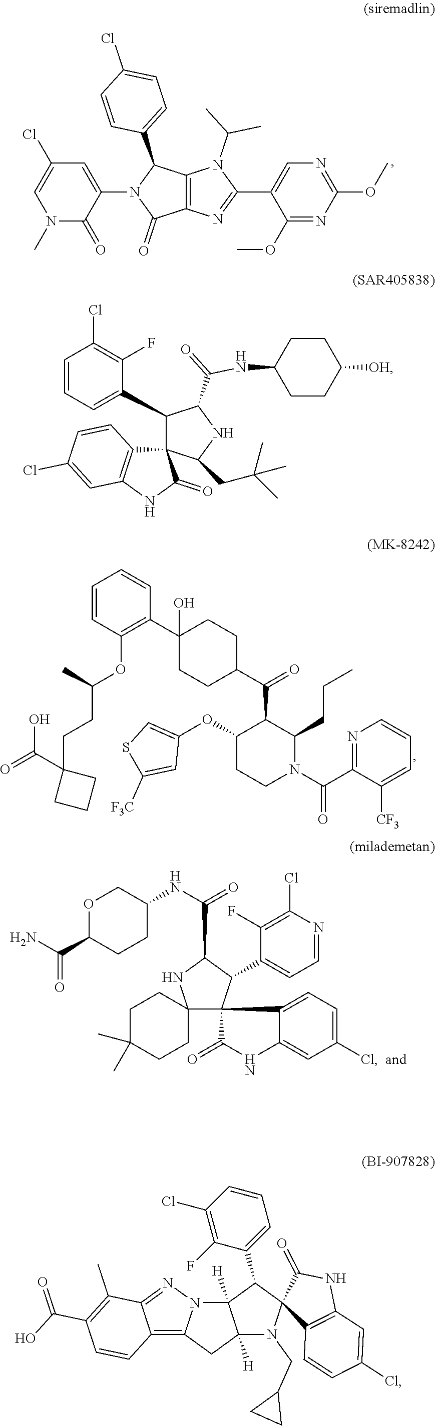

or a pharmaceutically-acceptable salt thereof.

Embodiment 37. The combination of any one of embodiments 30-36, wherein the compound that increases anti-tumor activity of the mutant p53 protein is

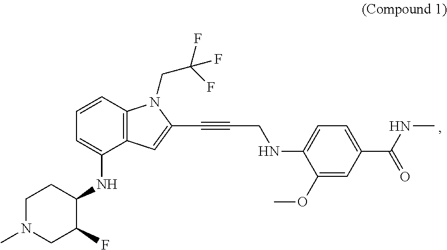

or a pharmaceutically-acceptable salt thereof.

Embodiment 38. A method of treating a solid tumor in a subject in need thereof, the method comprising:
(i) administering to the subject a therapeutically-effective amount of a compound that increases anti-tumor activity of a mutant p53 protein in the subject, wherein the compound is of the formula:

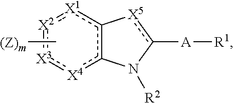

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each Z is independently -Q¹-N(R³)J, -Q¹-O-J, or -Q¹-J;
wherein at least one of X¹, X², X³, and X⁴ is a carbon atom connected to Q¹;
   A is a linking group;
   each Q¹ is independently alkylene, alkenylene, or alkynylene, each of which is independently substituted or unsubstituted, or C═O, C═S, C═CR¹⁴R¹⁵, C═NR¹⁴, or a bond;
   m is 1, 2, 3, or 4;
   each J is independently a cyclic group;
   R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
   each R³ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and J together with the nitrogen atom to which R³ and J are bound form a ring, wherein the ring is substituted or unsubstituted;
   each R², R⁵, R⁶, R⁷, R⁸, R⁹, R¹⁰, R¹¹, R¹², R¹³, R¹⁴, R⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
   each R¹⁹ and R²⁰ is alkyl, alkenyl, alkynyl, aryl, heteroaryl, or heterocyclyl, each of which is independently substituted or unsubstituted, or C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
   each R²¹ and R²² is independently alkyl, alkenyl, alkynyl, aryl, heteroaryl, or heterocyclyl, each of which is independently substituted or unsubstituted, or hydrogen; and
   each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof; and
(ii) administering to the subject a therapeutically-effective amount of a PI3K inhibitor.

Embodiment 39. The method of embodiment 38, wherein the PI3K inhibitor is alpelisib, or a pharmaceutically-acceptable salt thereof.

Embodiment 40. The method of embodiment 38, wherein the PI3K inhibitor is inavolisib, or a pharmaceutically-acceptable salt thereof.

Embodiment 41. The method of embodiment 38, wherein the PI3K inhibitor is serabelisib, or a pharmaceutically-acceptable salt thereof.

Embodiment 42. The method of embodiment 38, wherein the PI3K inhibitor is GSK2636771, or a pharmaceutically-acceptable salt thereof.

Embodiment 43. The method of embodiment 38, wherein the PI3K inhibitor is eganelisib, or a pharmaceutically-acceptable salt thereof.

Embodiment 44. The method of embodiment 38, wherein the PI3K inhibitor is idelalisib, or a pharmaceutically-acceptable salt thereof.

Embodiment 45. The method of embodiment 38, wherein the PI3K inhibitor is duvelisib, or a pharmaceutically-acceptable salt thereof.

Embodiment 46. The method of any one of embodiments 38-45, wherein the compound that increases anti-tumor activity of a mutant p53 protein is of the formula:

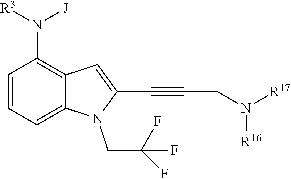

wherein R³ is H; J is piperidinyl, piperazinyl, tetahydropyranyl, morpholinyl, or pyrrolidinyl, each of which is independently substituted or unsubstituted; and each R¹⁶ and R¹⁷ is independently H, or alkyl, alkenyl, aryl, heteroaryl, or heterocyclyl, each of which is independently substituted or unsubstituted.

Embodiment 47. The method of embodiment 46, wherein each R¹⁶ and R¹⁷ is independently alkyl, alkenyl, aryl, heteroaryl, heterocyclyl, each of which is independently substituted or unsubstituted; or hydrogen.

Embodiment 48. The method of embodiment 46 or embodiment 47, wherein R¹⁶ is hydrogen or alkyl.

Embodiment 49. The method of any one of embodiments 46-48, wherein R¹⁷ is aryl, heteroaryl, or heterocyclyl, each of which is independently substituted or unsubstituted.

Embodiment 50. The method of any one of embodiments 46-49, wherein R¹⁷ is substituted aryl.

Embodiment 51. The method of any one of embodiments 46-50, wherein R¹⁷ is phenyl substituted with a sulfoxide group, sulfonyl group, carboxyl group, amide group, amino group, alkyl, alkoxy, hydroxy, or heterocyclyl, each of which is independently substituted or unsubstituted, or halo or cyano.

Embodiment 52. The method of any one of embodiments 38-48, wherein the compound that increases anti-tumor activity of the mutant p53 protein is

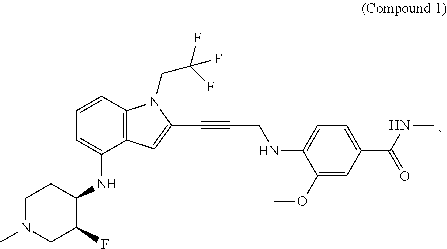

or a pharmaceutically-acceptable salt thereof.

Embodiment 53. A method of treating a solid tumor in a subject in need thereof, the method comprising:
(i) administering to the subject a therapeutically-effective amount of a compound that increases anti-tumor activity of a mutant p53 protein in the subject, wherein the compound is of the formula:

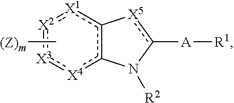

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
each Z is independently -Q¹-N(R³)J, -Q¹-O-J, or -Q¹-J;
wherein at least one of X¹, X², X³, and X⁴ is a carbon atom connected to Q¹;
A is a linking group;
each Q¹ is independently alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
each J is independently a cyclic group;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and J together with the nitrogen atom to which R³ and J are bound form a ring, wherein the ring is substituted or unsubstituted;
each R², R⁵, R⁶, R⁷, R⁸, R⁹, R¹⁰, R¹¹, R¹², R¹³, R¹⁴, R⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, hydrogen, or halogen;
each R¹⁹ and R²⁰ is alkyl, alkenyl, alkynyl, aryl, heteroaryl, or heterocyclyl, each of which is independently substituted or unsubstituted, or C(O)R²³, —C(O)OR²³, —C(O)NR²³R²⁴, —OR²³, —SR²³, —NR²³R²⁴, —NR²³C(O)R²⁴, —OC(O)R²³, hydrogen, or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof; and
(ii) administering to the subject a therapeutically-effective amount of a AKT inhibitor selected from MK2206, ipatasertib, and capivasertib.

Embodiment 54. The method of embodiment 53, wherein the AKT inhibitor is MK2206, or a pharmaceutically-acceptable salt thereof.

Embodiment 55. The method of embodiment 53, wherein the AKT inhibitor is ipatasertib, or a pharmaceutically-acceptable salt thereof.

Embodiment 56. The method of embodiment 53, wherein the AKT inhibitor is capivasertib, or a pharmaceutically-acceptable salt thereof.

Embodiment 57. The method of any one of embodiments 53-56, wherein the compound is of the formula:

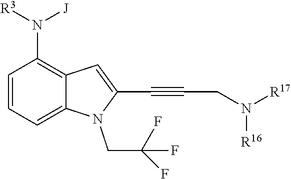

wherein R³ is H; J is piperidinyl, piperazinyl, tetahydropyranyl, morpholinyl, or pyrrolidinyl, each of which is independently substituted or unsubstituted; and each R¹⁶ and R¹⁷ is independently alkyl, alkenyl, aryl, heteroaryl, heterocyclyl, each of which is independently substituted, or unsubstituted, or H.

Embodiment 58. The method of embodiment 57, wherein each R¹⁶ and R¹⁷ is independently alkyl, alkenyl, aryl, heteroaryl, heterocyclyl, each of which is independently substituted or unsubstituted; or hydrogen.

Embodiment 59. The method of embodiment 57 or embodiment 58, wherein R¹⁶ is hydrogen or alkyl.

Embodiment 60. The method of any one of embodiments 57-59, wherein R¹⁷ is aryl, heteroaryl, or heterocyclyl, each of which is independently substituted or unsubstituted.

Embodiment 61. The method of any one of embodiments 57-60, wherein R¹⁷ is substituted aryl.

Embodiment 62. The method of any one of embodiments 57-61, wherein R¹⁷ is phenyl substituted 1, 2, 3, or 4 groups each independently selected from a sulfoxide group, carbamoyl group, sulfonyl group, carboxyl group, amide group, amino group, alkyl, alkoxy, hydroxy, and heterocyclyl, each of which is independently substituted or unsubstituted, or halo or cyano.

Embodiment 63. The method of any one of embodiments 53-62, wherein the compound that increases anti-tumor activity of the mutant p53 protein is

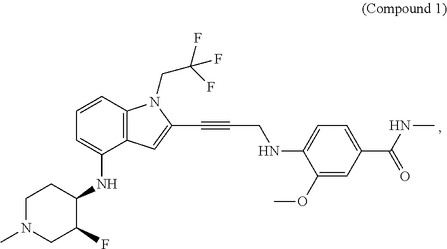

or a pharmaceutically-acceptable salt thereof.

### EXAMPLES

### Example 1: Compounds of the Disclosure

Indole compounds with alkynyl, aryl, and heteroaryl linkers were prepared. Alkynyl-linked indole compounds are shown in TABLE 1. Aryl-linked indole compounds are shown in TABLE 2. Heteroaryl-linked indole compounds are shown in TABLE 3.

TABLE 1
Alkynyl indole compounds of the disclosure.

| Mol # | IUPAC name |
|---|---|
| 1. | 1-Anilino-3-{1-ethyl-5-[(1-methyl-4-piperidylamino)methyl]-1H-indol-2-yl}-2- |
| | propyne |
| 2. | 1-Anilino-3-{1-ethyl-5-[(methylamino)methyl]-1H-indol-2-yl}-2-propyne |
| 3. | 1-Anilino-3-{1-ethyl-5-[(tetrahydro-2H-pyran-4-ylamino)methyl]-1H-indol-2-yl}- |
| | 2-propyne |
| 4. | 1-Anilino-3-[5-(benzylaminomethyl)-1-ethyl-1H-indol-2-yl]-2-propyne |
| 5. | 3-{1-Ethyl-5-[(methylamino)methyl]-1H-indol-2-yl}-1-(p-fluorophenylamino)-2- |
| | propyne |
| 6. | 3-{1-Ethyl-5-[(tetrahydro-2H-pyran-4-ylmino)methyl]-1H-indol-2-yl}-1-(p- |
| | fluorophenylamino)-2-propyne |
| 7. | 1-(p-Chlorophenylamino)-3-{1-ethyl-5-[(1-methyl-4-piperidylamino)methyl]-1H- |
| | indol-2-yl}-2-propyne |
| 8. | 3-{1-Ethyl-5-[(tetrahydro-2H-pyran-4-ylamino)methyl]-1H-indol-2-yl}-1-(6- |
| | methyl-3-pyridylamino)-2-propyne |
| 9. | 3-{1-Ethyl-5-[(methylamino)methyl]-1H-indol-2-yl}-1-(6-methyl-3- |
| | pyridylamino)-2-propyne |
| 10. | 3-{1-Ethyl-5-[(1-methyl-4-piperidylamino)methyl]-1H-indol-2-yl}-1-(2-methyl-4- |
| | pyridylamino)-2-propyne |
| 11. | 3-[5-(Benzylaminomethyl)-1-ethyl-1H-indol-2-yl]-1-(2-methyl-4-pyridylamino)- |
| | 2-propyne |
| 12. | N-(3-{5-[(Diethylamino)methyl]-1-ethyl-1H-indol-2-yl}prop-2-yn-1-yl)aniline |
| 13. | 4-Chloro-N-(3-{5-[(diethylamino)methyl]-1-ethyl-1H-indol-2-yl}prop-2-yn-1- |
| | yl)aniline |
| 14. | N-({1-Ethyl-2-[3-(phenylamino)prop-1-yn-1-yl]-1H-indol-5-yl}methyl)oxetan-3- |
| | amine |
| 15. | N-[3-(1-Ethyl-5-{[(2-methylpropyl)amino]methyl}-1H-indol-2-yl)prop-2-yn-1- |
| | yl]aniline |
| 16. | N-[3-(1-Ethyl-5-{[(2-methoxyethyl)amino]methyl}-1H-indol-2-yl)prop-2-yn-1- |
| | yl]aniline |
| 17. | N-({1-ethyl-2-[3-(phenylamino)prop-1-yn-1-yl]-1H-indol-5-yl}methyl)-1- |
| | methanesulfonylpiperidin-4-amine |
| 18. | N-(3-{1-Ethyl-5-[(ethylamino)methyl]-1H-indol-2-yl}prop-2-yn-1-yl)aniline |
| 19. | N-{3-[5-({[2-(Dimethylamino)ethyl]amino}methyl)-1-ethyl-1H-indol-2-yl]prop- |
| | 2-yn-1-yl}aniline |
| 20. | 6-tert-Butyl-N-[3-(1-ethyl-5-{[(1-methylpiperidin-4-yl)amino]methyl}-1H-indol- |
| | 2-yl)prop-2-yn-1-yl]pyridin-3-amine |
| 21. | N-[(2-{3-[(4-Chlorophenyl)amino]prop-1-yn-1-yl}-1-ethyl-1H-indol-5- |
| | yl)methyl]oxan-4-amine |
| 22. | 6-tert-Butyl-N-(3-{1-ethyl-5-[(methylamino)methyl]-1H-indol-2-yl}prop-2-yn-1- |
| | yl)pyridin-3-amine |
| 23. | 4-[(3-{1-Ethyl-5-[(methylamino)methyl]-1H-indol-2-yl}prop-2-yn-1- |
| | yl)amino]benzonitrile |
| 24. | 4-tert-Butyl-N-(3-{1-ethyl-5-[(methylamino)methyl]-1H-indol-2-yl}prop-2-yn-1- |
| | yl)benzamide |
| 25. | 4-Chloro-N-(3-{1-ethyl-5-[(methylamino)methyl]-1H-indol-2-yl}prop-2-yn-1-yl)- |
| | 3-fluorobenzamide |
| 26. | 4-Cyano-N-({1-ethyl-2-[3-(phenylformamido)prop-1-yn-1-yl]-1H-indol-5- |
| | yl}methyl)-N-methylbenzamide |
| 27. | 3-(3-{1-Ethyl-5-[(methylamino)methyl]-1H-indol-2-yl}prop-2-yn-1-yl)-1-[4- |
| | (trifluoromethyl)phenyl]urea |
| 28. | N-{[1-(2-Chloroethyl)-2-{3-[(4-chlorophenyl)amino]prop-1-yn-1-yl}-1H-indol-5- |
| | yl]methyl}oxan-4-amine |
| 29. | 2-(4-{[3-(1-Ethyl-5-{[(1-methylpiperidin-4-yl)amino]methyl}-1H-indol-2- |
| | yl)prop-2-yn-1-yl]amino}phenyl)-2-methylpropanenitrile |
| 30. | 4-Cyano-N-(3-{1-ethyl-5-[(methylamino)methyl]-1H-indol-2-yl}prop-2-yn-1- |
| | yl)benzamide |
| 31. | N-(3-{1-Ethyl-5-[(methylamino)methyl]-1H-indol-2-yl}prop-2-yn-1-yl)-6- |
| | methylpyridine-3-carboxamide |
| 32. | 3-[3-(1-Ethyl-5-{[(1-methylpiperidin-4-yl)amino]methyl}-1H-indol-2-yl)prop-2- |
| | yn-1-yl]-1-phenylurea |
| 33. | N-[(2-{3-[(4-Chloro-3-fluorophenyl)amino]prop-1-yn-1-yl}-1-ethyl-1H-indol-5- |
| | yl)methyl]-1-methylpiperidin-4-amine |
| 34. | 2-(5-{[3-(1-Ethyl-5-{[(1-methylpiperidin-4-yl)amino]methyl}-1H-indol-2- |
| | yl)prop-2-yn-1-yl]amino}pyridin-2-yl)-2-methylpropanenitrile |
| 35. | N-{[1-(2-Chloroethyl)-2-{3-[(4-chlorophenyl)amino]prop-1-yn-1-yl}-1H-indol-5- |
| | yl]methyl}-1-methylpiperidin-4-amine |
| 36. | 6-tert-Butyl-N-[3-(1-ethyl-5-{[(1-methanesulfonylpiperidin-4-yl)amino]methyl}- |
| | 1H-indol-2-yl)prop-2-yn-1-yl]pyridin-3-amine |
| 37. | 2-(4-{[3-(1-Ethyl-5-{[(1-methylpiperidin-4-yl)amino]methyl}-1H-indol-2- |
| | yl)prop-2-yn-1-yl]amino}phenyl)-2-methylpropanoic acid |
| 38. | 3-(1-Ethyl-5-{[(1-methylpiperidin-4-yl)amino]methyl}-1H-indol-2-yl)-N- |
| | methylprop-2-ynamide |
| 39. | Ethyl 2-(4-{[3-(1-ethyl-5-{[(1-methylpiperidin-4-yl)amino]methyl}-1H-indol-2- |
| | yl)prop-2-yn-1-yl]amino}phenyl)-2-methylpropanoate |
| 40. | 2-(5-{[3-(1-Ethyl-5-{[(oxan-4-yl)amino]methyl}-1H-indol-2-yl)prop-2-yn-1- |
| | yl]amino}pyridin-2-yl)-2-methylpropanenitrile |
| 41. | N-[(1-Ethyl-2-{3-[(4-methylphenyl)amino]prop-1-yn-1-yl}-1H-indol-5- |
| | yl)methyl]-1-methylpiperidin-4-amine |
| 42. | 4-{[3-(1-Ethyl-5-{[(1-methylpiperidin-4-yl)amino]methyl}-1H-indol-2-yl)prop-2- |
| | yn-1-yl]amino}benzonitrile |
| 43. | 3-(1-Ethyl-5-{[(1-methylpiperidin-4-yl)amino]methyl}-1H-indol-2-yl)-N- |
| | phenylprop-2-ynamide |
| 44. | N-[(2-{3-[(4-Chlorophenyl)amino]prop-1-yn-1-yl}-1-ethyl-1H-indol-5- |
| | yl)methyl]-1-methanesulfonylpiperidin-4-amine |
| 45. | 1-(4-{[(2-{3-[(4-Chlorophenyl)amino]prop-1-yn-1-yl}-1-ethyl-1H-indol-5- |
| | yl)methyl]amino}piperidin-1-yl)ethan-1-one |
| 46. | 6-tert-Butyl-N-[3-(1-ethyl-5-{[(1-methylpiperidin-4-yl)amino]methyl}-1H-indol- |
| | 2-yl)prop-2-yn-1-yl]pyridine-3-carboxamide |
| 47. | N-(3-{1-Ethyl-5-[(methylamino)methyl]-1H-indol-2-yl}prop-2-yn-1-yl)-4- |
| | (trifluoromethyl)aniline |
| 48. | N-[(1-Ethyl-2-{3-[(4-methylphenyl)amino]prop-1-yn-1-yl}-1H-indol-5- |
| | yl)methyl]oxan-4-amine |
| 49. | N-(3-{1-ethyl-4-[(methylamino)methyl]-1H-indol-2-yl}prop-2-yn-1-yl)aniline |
| 50. | N-[3-(1-ethyl-4-{[4-(pyrrolidin-1-yl)piperidin-1-yl]methyl}-1H-indol-2-yl)prop-2- |
| | yn-1-yl]aniline |
| 51. | N-({1-ethyl-2-[3-(phenylamino)prop-1-yn-1-yl]-1H-indol-4-yl}methyl)-1- |
| | methylpiperidin-4-amine |
| 52. | 1-[(2-{3-[(4-chlorophenyl)amino]prop-1-yn-1-yl}-1-ethyl-1H-indol-4- |
| | yl)methyl]piperidin-4-ol |
| 53. | 4-Chloro-N-[3-(1-ethyl-4-{[4-(pyrrolidin-1-yl)piperidin-1-yl]methyl}-1H-indol-2- |
| | yl)prop-2-yn-1-yl]aniline |
| 54. | 1-[(2-{3-[(4-chlorophenyl)amino]prop-1-yn-1-yl}-1-ethyl-1H-indol-4-yl)methyl]- |
| | N,N-dimethylpiperidin-4-amine |
| 55. | 4-Chloro-N-(3-{1-ethyl-4-[(4-methylpiperazin-1-yl)methyl]-1H-indol-2-yl}prop- |
| | 2-yn-1-yl)aniline |
| 56. | 1-{1-[(2-{3-[(4-Chlorophenyl)amino]prop-1-yn-1-yl}-1-ethyl-1H-indol-4- |
| | yl)methyl]piperidin-4-yl}piperidin-4-ol |
| 57. | 2-(5-{[3-(4-{[4-(4-Aminopiperidin-1-yl)piperidin-1-yl]methyl}-1-ethyl-1H-indol- |
| | 2-yl)prop-2-yn-1-yl]amino}pyridin-2-yl)-2-methylpropanenitrile |
| 58. | 1-[(1-ethyl-2-{3-[(4-fluorophenyl)amino]prop-1-yn-1-yl}-1H-indol-5-yl)methyl]- |
| | N,N-dimethylpiperidin-4-amine |
| 59. | 4-N-({1-ethyl-2-[3-(phenylamino)prop-1-yn-1-yl]-1H-indol-5-yl}methyl)-1-N,1- |
| | N-dimethylcyclohexane-1,4-diamine |
| 60. | 4-chloro-N-(3-{1-ethyl-5-[(methylamino)methyl]-1H-indol-2-yl}prop-2-yn-1-yl)- |
| | 3-fluoroaniline |
| 61. | 6-tert-butyl-N-(3-{1-ethyl-5-[(methylamino)methyl]-1H-indol-2-yl}prop-2-yn-1- |
| | yl)pyridine-3-carboxamide |
| 62. | N-(3-{1-ethyl-5-[(methylamino)methyl]-1H-indol-2-yl}prop-2-yn-1-yl)benzamide |
| 63. | 3-(3-{1-ethyl-5-[(methylamino)methyl]-1H-indol-2-yl}prop-2-yn-1-yl)-1-(4- |
| | methylphenyl)urea |
| 64. | 4-chloro-N-(3-{1-ethyl-5-[(methylamino)methyl]-1H-indol-2-yl}prop-2-yn-1- |
| | yl)aniline |
| 65. | 4-{[3-(1-ethyl-5-{[(oxan-4-yl)amino]methyl}-1H-indol-2-yl)prop-2-yn-1- |
| | yl]amino}benzonitrile |
| 66. | N-[(2-{3-[(4-chloro-3-fluorophenyl)amino]prop-1-yn-1-yl}-1-ethyl-1H-indol-5- |
| | yl)methyl]oxan-4-amine |
| 67. | 3-[3-(1-ethyl-5-{[(oxan-4-yl)amino]methyl}-1H-indol-2-yl)prop-2-yn-1-yl]-1- |
| | phenylurea |
| 68. | 6-tert-butyl-N-[3-(1-ethyl-5-{[(oxan-4-yl)amino]methyl}-1H-indol-2-yl)prop-2- |
| | yn-1-yl]pyridin-3-amine |
| 69. | 4-{[(2-{3-[(4-chlorophenyl)amino]prop-1-yn-1-yl}-1-ethyl-1H-indol-5- |
| | yl)methyl]amino}-1λ⁶-thiane-1,1-dione |
| 70. | N-[(2-{3-[(4-chlorophenyl)amino]prop-1-yn-1-yl}-1-ethyl-1H-indol-5-yl)methyl]- |
| | 1-(2-methanesulfonylethyl)piperidin-4-amine |
| 71. | 1-(4-{[(2-{3-[(4-chlorophenyl)amino]prop-1-yn-1-yl}-1-ethyl-1H-indol-5- |
| | yl)methyl]amino}piperidin-1-yl)-2-(dimethylamino)ethan-1-one |
| 72. | 2-(4-{[(2-{3-[(4-chlorophenyl)amino]prop-1-yn-1-yl}-1-ethyl-1H-indol-5- |
| | yl)methyl]amino}piperidin-1-yl)-N,N-dimethylacetamide |
| 73. | 2-tert-butyl-N-[3-(1-ethyl-5-{[(1-methylpiperidin-4-yl)amino]methyl}-1H-indol- |
| | 2-yl)prop-2-yn-1-yl]pyrimidin-5-amine |
| 74. | 2-methyl-2-(5-{[3-(5-{[(oxan-4-yl)amino]methyl}-1-(2,2,2-trifluoroethyl)-1H- |
| | indol-2-yl)prop-2-yn-1-yl]amino}pyridin-2-yl)propanenitrile |
| 75. | 2-[5-({3-[1-(2-fluoroethyl)-5-{[(oxan-4-yl)amino]methyl}-1H-indol-2-yl]prop-2- |
| | yn-1-yl}amino)pyridin-2-yl]-2-methylpropanenitrile |
| 76. | 3-(1-ethyl-5-{[(1-methylpiperidin-4-yl)amino]methyl}-1H-indol-2-yl)prop-2-yn- |
| | 1-ol |
| 77. | 2-[5-({3-[1-(2-chloroethyl)-5-{[(oxan-4-yl)amino]methyl}-1H-indol-2-yl]prop-2- |
| | yn-1-yl}amino)pyridin-2-yl]-2-methylpropanenitrile |
| 78. | 2-[5-({3-[1-(2,2-difluoroethyl)-5-{[(oxan-4-yl)amino]methyl}-1H-indol-2- |
| | yl]prop-2-yn-1-yl}amino)pyridin-2-yl]-2-methylpropanenitrile |
| 79. | 6-chloro-N-[3-(1-ethyl-5-{[(1-methylpiperidin-4-yl)amino]methyl}-1H-indol-2- |
| | yl)prop-2-yn-1-yl]pyridin-3-amine |
| 80. | tert-butyl N-({3-ethyl-2-[3-(phenylamino)prop-1-yn-1-yl]-1H-indol-6-yl}methyl)- |
| | N-(oxan-4-yl)carbamate |
| 81. | 6-Chloro-N-[3-(1-ethyl-4-{[4-(pyrrolidin-1-yl)piperidin-1-yl]methyl}-1H-indol-2- |
| | yl)prop-2-yn-1-yl]pyridin-3-amine |
| 82. | 3-(1-ethyl-5-{[(1-methylpiperidin-4-yl)amino]methyl}-1H-indol-2-yl)prop-2-yn- |
| | 1-yl benzoate |
| 83. | 2-[5-({3-[1-(2-chloroethyl)-5-{[(1-methylpiperidin-4-yl)amino]methyl}-1H-indol- |
| | 2-yl]prop-2-yn-1-yl}amino)pyridin-2-yl]-2-methylpropanenitrile |
| 84. | N-(6-chloropyridin-3-yl)-3-(1-ethyl-5-{[(1-methylpiperidin-4-yl)amino]methyl}- |
| | 1H-indol-2-yl)prop-2-ynamide |
| 85. | N-[6-(1-cyano-1-methylethyl)pyridin-3-yl]-3-(1-ethyl-5-{[(1-methylpiperidin-4- |
| | yl)amino]methyl}-1H-indol-2-yl)prop-2-ynamide |
| 86. | N-({3-ethyl-2-[3-(phenylamino)prop-1-yn-1-yl]-1H-indol-6-yl}methyl)oxan-4- |
| | amine |
| 87. | 2-[5-({3-[1-(2-chloroethyl)-4-{[4-(pyrrolidin-1-yl)piperidin-1-yl]methyl}-1H- |
| | indol-2-yl]prop-2-yn-1-yl}amino)pyridin-2-yl]-2-methylpropanenitrile |
| 88. | 2-(5-{[3-(5-{[(1-methanesulfonylpiperidin-4-yl)amino]methyl}-1-(2,2,2- |
| | trifluoroethyl)-1H-indol-2-yl)prop-2-yn-1-yl]amino}pyridin-2-yl)-2- |
| | methylpropanenitrile |
| 89. | 2-[5-({3-[5-({[1-(2-methanesulfonylethyl)piperidin-4-yl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 90. | 2-(5-{[3-(5-{[(1-acetylpiperidin-4-yl)amino]methyl}-1-(2,2,2-trifluoroethyl)-1H- |
| | indol-2-yl)prop-2-yn-1-yl]amino}pyridin-2-yl)-2-methylpropanenitrile |
| 91. | 2-{5-[(3-{5-[({1-[2-(dimethylamino)acetyl]piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 92. | 2-methyl-2-(5-{[3-(5-{[(1-methylpiperidin-4-yl)amino]methyl}-1-(2,2,2- |
| | trifluoroethyl)-1H-indol-2-yl)prop-2-yn-1-yl]amino}pyridin-2-yl)propanenitrile |
| 93. | 2-methyl-2-{5-[(3-{5-[(methylamino)methyl]-1-(2,2,2-trifluoroethyl)-1H-indol-2- |
| | yl}prop-2-yn-1-yl)amino]pyridin-2-yl}propanenitrile |
| 94. | 6-Chloro-N-[3-(5-{[(1-methylpiperidin-4-yl)amino]methyl}-1-(2,2,2- |
| | trifluoroethyl)-1H-indol-2-yl)prop-2-yn-1-yl]pyridin-3-amine |
| 95. | 6-chloro-N-[3-(5-{[(oxan-4-yl)amino]methyl}-1-(2,2,2-trifluoroethyl)-1H-indol-2- |
| | yl)prop-2-yn-1-yl]pyridin-3-amine |
| 96. | 2-[5-({3-[1-(cyclopropylmethyl)-5-{[(oxan-4-yl)amino]methyl}-1H-indol-2- |
| | yl]prop-2-yn-1-yl}amino)pyridin-2-yl]-2-methylpropanenitrile |
| 97. | 2-(5-{[3-(4-{[4-(diethylamino)piperidin-1-yl]methyl}-1-(2,2,2-trifluoroethyl)-1H- |
| | indol-2-yl)prop-2-yn-1-yl]amino}pyridin-2-yl)-2-methylpropanenitrile |
| 98. | 2-methyl-2-{5-[(3-{4-[(4-methylpiperazin-1-yl)methyl]-1-(2,2,2-trifluoroethyl)- |
| | 1H-indol-2-yl}prop-2-yn-1-yl)amino]pyridin-2-yl}propanenitrile |
| 99. | 2-(5-{[3-(1-ethyl-7-fluoro-4-{[4-(pyrrolidin-1-yl)piperidin-1-yl]methyl}-1H- |
| | indol-2-yl)prop-2-yn-1-yl]amino}pyridin-2-yl)-2-methylpropanenitrile |
| 100. | 2-methyl-2-(5-{[3-(4-{[4-(pyrrolidin-1-yl)piperidin-1-yl]methyl}-1-(2,2,2- |
| | trifluoroethyl)-1H-indol-2-yl)prop-2-yn-1-yl]amino}pyridin-2-yl)propanenitrile |
| 101. | 2-(5-{[3-(4-{[4-(4-hydroxypiperidin-1-yl)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 102. | N-(6-cyanopyridin-3-yl)-3-(1-ethyl-5-{[(1-methylpiperidin-4-yl)amino]methyl}- |
| | 1H-indol-2-yl)prop-2-ynamide |
| 103. | N-[6-(1-cyano-1-methylethyl)pyridin-3-yl]-3-(5-{[(oxan-4-yl)amino]methyl}-1- |
| | (2,2,2-trifluoroethyl)-1H-indol-2-yl)prop-2-ynamide |
| 104. | N-[6-(1-cyano-1-methylethyl)pyridin-3-yl]-3-(5-{[(1-methylpiperidin-4- |
| | yl)amino]methyl}-1-(2,2,2-trifluoroethyl)-1H-indol-2-yl)prop-2-ynamide |
| 105. | 2-methyl-2-(5-{[3-(5-{[(oxan-4-yl)amino]methyl}-1-(oxiran-2-ylmethyl)-1H- |
| | indol-2-yl)prop-2-yn-1-yl]amino}pyridin-2-yl)propanenitrile |
| 106. | 2-(5-{[3-(5-{[(2-methoxyethyl)amino]methyl}-1-(2,2,2-trifluoroethyl)-1H-indol- |
| | 2-yl)prop-2-yn-1-yl]amino}pyridin-2-yl)-2-methylpropanenitrile |
| 107. | 2-methyl-2-[5-({3-[5-({[2-(morpholin-4-yl)ethyl]amino}methyl)-1-(2,2,2- |
| | trifluoroethyl)-1H-indol-2-yl]prop-2-yn-1-yl}amino)pyridin-2-yl]propanenitrile |
| 108. | 2-methyl-2-(5-{[3-(4-{[(1-methylpiperidin-4-yl)amino]methyl}-1-(2,2,2- |
| | trifluoroethyl)-1H-indol-2-yl)prop-2-yn-1-yl]amino}pyridin-2-yl)propanenitrile |
| 109. | 2-methyl-2-(5-{[3-(4-{[(oxan-4-yl)amino]methyl}-1-(2,2,2-trifluoroethyl)-1H- |
| | indol-2-yl)prop-2-yn-1-yl]amino}pyridin-2-yl)propanenitrile |
| 110. | 2-[5-({3-[5-({[2-(dimethylamino)ethyl]amino}methyl)-1-(2,2,2-trifluoroethyl)- |
| | 1H-indol-2-yl]prop-2-yn-1-yl}amino)pyridin-2-yl]-2-methylpropanenitrile |
| 111. | 2-(5-{[3-(7-fluoro-4-{[4-(pyrrolidin-1-yl)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 112. | 2-methyl-2-[5-({3-[1-(2,2,2-trifluoroethyl)-5-{[(2,2,2- |
| | trifluoroethyl)amino]methyl}-1H-indol-2-yl]prop-2-yn-1-yl}amino)pyridin-2- |
| | yl]propanenitrile |
| 113. | 2-[5-({3-[5-({[1-(2-hydroxyethyl)piperidin-4-yl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 114. | 2-[5-({3-[5-({[1-(2-methoxyethyl)piperidin-4-yl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 115. | 2-[5-({3-[5-({[4-(dimethylamino)cyclohexyl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 116. | 2-methyl-2-{5-[(3-{5-[({1-[2-(morpholin-4-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}propanenitrile |
| 117. | 2-(5-{[3-(4-{[(2-methoxyethyl)amino]methyl}-1-(2,2,2-trifluoroethyl)-1H-indol- |
| | 2-yl)prop-2-yn-1-yl]amino}pyridin-2-yl)-2-methylpropanenitrile |
| 118. | 2-methyl-2-{5-[(3-{4-[(methylamino)methyl]-1-(2,2,2-trifluoroethyl)-1H-indol-2- |
| | yl}prop-2-yn-1-yl)amino]pyridin-2-yl}propanenitrile |
| 119. | 2-{5-[(3-{4-[(4-acetylpiperazin-1-yl)methyl]-1-(2,2,2-trifluoroethyl)-1H-indol-2- |
| | yl}prop-2-yn-1-yl)amino]pyridin-2-yl}-2-methylpropanenitrile |
| 120. | 2-methyl-2-[5-({3-[4-(morpholin-4-ylmethyl)-1-(2,2,2-trifluoroethyl)-1H-indol-2- |
| | yl]prop-2-yn-1-yl}amino)pyridin-2-yl]propanenitrile |
| 121. | 2-(5-{[3-(4-{[4-(dimethylamino)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 122. | 2-[5-({3-[4-(hydroxymethyl)-1-(2,2,2-trifluoroethyl)-1H-indol-2-yl]prop-2-yn-1- |
| | yl}amino)pyridin-2-yl]-2-methylpropanenitrile |
| 123. | 2-methyl-2-[5-({3-[4-({4-[2-(morpholin-4-yl)-2-oxoethyl]piperazin-1-yl}methyl)- |
| | 1-(2,2,2-trifluoroethyl)-1H-indol-2-yl]prop-2-yn-1-yl}amino)pyridin-2- |
| | yl]propanenitrile |
| 124. | 2-(5-{[3-(3-ethyl-7-{[4-(pyrrolidin-1-yl)piperidin-1-yl]methyl}-1H-indol-2- |
| | yl)prop-2-yn-1-yl]amino}pyridin-2-yl)-2-methylpropanenitrile |
| 125. | methyl 5-{[3-(5-{[(oxan-4-yl)amino]methyl}-1-(2,2,2-trifluoroethyl)-1H-indol-2- |
| | yl)prop-2-yn-1-yl]amino}pyridine-2-carboxylate |
| 126. | N-methyl-5-{[3-(5-{[(oxan-4-yl)amino]methyl}-1-(2,2,2-trifluoroethyl)-1H-indol- |
| | 2-yl)prop-2-yn-1-yl]amino}pyridine-2-carboxamide |
| 127. | N-(2-hydroxyethyl)-5-{[3-(5-{[(oxan-4-yl)amino]methyl}-1-(2,2,2-trifluoroethyl)- |
| | 1H-indol-2-yl)prop-2-yn-1-yl]amino}pyridine-2-carboxamide |
| 128. | N-(2-methoxyethyl)-5-{[3-(5-{[(oxan-4-yl)amino]methyl}-1-(2,2,2- |
| | trifluoroethyl)-1H-indol-2-yl)prop-2-yn-1-yl]amino}pyridine-2-carboxamide |
| 129. | 2-[(5-{[3-(5-{[(oxan-4-yl)amino]methyl}-1-(2,2,2-trifluoroethyl)-1H-indol-2- |
| | yl)prop-2-yn-1-yl]amino}pyridin-2-yl)formamido]acetic acid |
| 130. | 5-{[3-(5-{[(oxan-4-yl)amino]methyl}-1-(2,2,2-trifluoroethyl)-1H-indol-2-yl)prop- |
| | 2-yn-1-yl]amino}pyridine-2-carboxylic acid |
| 131. | N-(2-methanesulfonylethyl)-5-{[3-(5-{[(oxan-4-yl)amino]methyl}-1-(2,2,2- |
| | trifluoroethyl)-1H-indol-2-yl)prop-2-yn-1-yl]amino}pyridine-2-carboxamide |
| 132. | 2-[5-({3-[1-(cyanomethyl)-5-{[(oxan-4-yl)amino]methyl}-1H-indol-2-yl]prop-2- |
| | yn-1-yl}amino)pyridin-2-yl]-2-methylpropanenitrile |
| 133. | 2-methyl-2-[5-({3-[1-(2-methylpropyl)-5-{[(oxan-4-yl)amino]methyl}-1H-indol- |
| | 2-yl]prop-2-yn-1-yl}amino)pyridin-2-yl]propanenitrile |
| 134. | 2-methyl-2-{5-[(3-{4-[(oxan-4-yl)amino]-1-(2,2,2-trifluoroethyl)-1H-indol-2- |
| | yl}prop-2-yn-1-yl)amino]pyridin-2-yl}propanenitrile |
| 135. | 5-{[3-(5-{[(oxan-4-yl)amino]methyl}-1-(2,2,2-trifluoroethyl)-1H-indol-2-yl)prop- |
| | 2-yn-1-yl]amino}pyridine-2-carbonitrile |
| 136. | N,N-dimethyl-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 137. | N-(oxan-4-yl)-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 138. | 2-tert-butyl-N-[3-(5-{[(oxan-4-yl)amino]methyl}-1-(2,2,2-trifluoroethyl)-1H- |
| | indol-2-yl)prop-2-yn-1-yl]pyrimidin-5-amine |
| 139. | N-(1-methylpiperidin-4-yl)-5-{[3-(5-{[(oxan-4-yl)amino]methyl}-1-(2,2,2- |
| | trifluoroethyl)-1H-indol-2-yl)prop-2-yn-1-yl]amino}pyridine-2-carboxamide |
| 140. | N-[6-(1-cyano-1-methylethyl)pyridin-3-yl]-3-[1-(2-fluoroethyl)-5-{[(oxan-4- |
| | yl)amino]methyl}-1H-indol-2-yl]prop-2-ynamide |
| 141. | 2-(5-{[3-(7-chloro-4-{[4-(pyrrolidin-1-yl)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 142. | 2-(5-{[3-(6-fluoro-4-{[4-(pyrrolidin-1-yl)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 143. | 2-(5-{[3-(1-ethyl-5-{[(oxan-4-yl)amino]methyl}-1H-pyrrolo[2,3-c]pyridin-2- |
| | yl)prop-2-yn-1-yl]amino}pyridin-2-yl)-2-methylpropanenitrile |
| 144. | 2-methyl-2-(5-{[3-(5-{[(oxan-4-yl)amino]methyl}-1-(2,2,2-trifluoroethyl)-1H- |
| | pyrrolo[2,3-c]pyridin-2-yl)prop-2-yn-1-yl]amino}pyridin-2-yl)propanenitrile |
| 145. | 2-(5-{[3-(5-{[4-(dimethylamino)piperidin-1-yl]methyl}-1-(2,2,2-trifluoroethyl)- |
| | 1H-pyrrolo[2,3-c]pyridin-2-yl)prop-2-yn-1-yl]amino}pyridin-2-yl)-2- |
| | methylpropanenitrile |
| 146. | 2-(5-{[3-(7-chloro-4-{[4-(pyrrolidin-1-yl)piperidin-1-yl]methyl}-1-(2,2,2- |
| | trifluoroethyl)-1H-pyrrolo[2,3-c]pyridin-2-yl)prop-2-yn-1-yl]amino}pyridin-2-yl)- |
| | 2-methylpropanenitrile |
| 147. | 2-(5-{[3-(4-{[4-(dimethylamino)-piperidin-1-yl]methyl}-6-fluoro-1-(2,2,2- |
| | trifluoroethyl)-1H-indol-2-yl)prop-2-yn-1-yl]amino}pyridin-2-yl)-2- |
| | methylpropanenitrile |
| 148. | 2-(5-{[3-(4-{[4-(diethylamino)piperidin-1-yl]methyl}-6-fluoro-1-(2,2,2- |
| | trifluoroethyl)-1H-indol-2-yl)prop-2-yn-1-yl]amino}pyridin-2-yl)-2- |
| | methylpropanenitrile |
| 149. | 2-(5-{[3-(6-fluoro-4-{[4-(4-hydroxypiperidin-1-yl)piperidin-1-yl]methyl}-1- |
| | (2,2,2-trifluoroethyl)-1H-indol-2-yl)prop-2-yn-1-yl]amino}pyridin-2-yl)-2- |
| | methylpropanenitrile |
| 150. | 2-(5-{[3-(6-fluoro-5-{[(oxan-4-yl)amino]methyl}-1-(2,2,2-trifluoroethyl)-1H- |
| | indol-2-yl)prop-2-yn-1-yl]amino}pyridin-2-yl)-2-methylpropanenitrile |
| 151. | 2-(5-{[3-(5-{[(1-acetylpiperidin-4-yl)amino]methyl}-6-fluoro-1-(2,2,2- |
| | trifluoroethyl)-1H-indol-2-yl)prop-2-yn-1-yl]amino}pyridin-2-yl)-2- |
| | methylpropanenitrile |
| 152. | 2-(5-{[3-(6-chloro-4-{[4-(dimethylamino)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 153. | 2-(5-{[3-(6-chloro-4-{[4-(diethylamino)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 154. | 2-(5-{[3-(6-chloro-4-{[4-(4-hydroxypiperidin-1-yl)piperidin-1-yl]methyl}-1- |
| | (2,2,2-trifluoroethyl)-1H-indol-2-yl)prop-2-yn-1-yl]amino}pyridin-2-yl)-2- |
| | methylpropanenitrile |
| 155. | 2-(5-{[3-(4-{[4-(2-methanesulfonyl-ethyl)piperazin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 156. | 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]methyl}piperazin-1-yl)-N,N- |
| | dimethylacetamide |
| 157. | 2-methyl-2-{5-[(3-{4-[(3-oxopiperazin-1-yl)methyl]-1-(2,2,2-trifluoroethyl)-1H- |
| | indol-2-yl}prop-2-yn-1-yl)amino]pyridin-2-yl}propanenitrile |
| 158. | 2-methyl-2-[5-({3-[4-({4-[2-(morpholin-4-yl)-2-oxoethyl]piperidin-1-yl}methyl)- |
| | 1-(2,2,2-trifluoroethyl)-1H-indol-2-yl]prop-2-yn-1-yl}amino)pyridin-2- |
| | yl]propanenitrile |
| 159. | 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]methyl}piperazin-1-yl)acetamide |
| 160. | N-[6-(1-cyano-1-methylethyl)pyridin-3-yl]-3-(4-{[4-(pyrrolidin-1-yl)piperidin-1- |
| | yl]methyl}-1-(2,2,2-trifluoroethyl)-1H-indol-2-yl)prop-2-ynamide |
| 161. | 2-(1-{[2-(3-{[6-(1-cyano-1-methylethyl)pyridin-3-yl]amino}prop-1-yn-1-yl)-1- |
| | (2,2,2-trifluoroethyl)-1H-indol-4-yl]methyl}piperidin-4-yl)acetamide |
| 162. | 2-(5-{[3-(4-{[4-(2-aminoethyl)-piperazin-1-yl]methyl}-1-(2,2,2-trifluoroethyl)- |
| | 1H-indol-2-yl)prop-2-yn-1-yl]amino}pyridin-2-yl)-2-methylpropanenitrile |
| 163. | 2-(1-{[2-(3-{[6-(1-cyano-1-methylethyl)pyridin-3-yl]amino}prop-1-yn-1-yl)-1- |
| | (2,2,2-trifluoroethyl)-1H-indol-4-yl]methyl}piperidin-4-yl)-N,N- |
| | dimethylacetamide |
| 164. | 2-methyl-2-(5-{[3-(4-{[4-(morpholin-4-yl)piperidin-1-yl]methyl}-1-(2,2,2- |
| | trifluoroethyl)-1H-indol-2-yl)prop-2-yn-1-yl]amino}pyridin-2-yl)propanenitrile |
| 165. | 2-(5-{[3-(4-{[4-(4-aminopiperidin-1-yl)piperidin-1-yl]methyl}-1-(2,2,2- |
| | trifluoroethyl)-1H-indol-2-yl)prop-2-yn-1-yl]amino}pyridin-2-yl)-2- |
| | methylpropanenitrile |
| 166. | 2-methyl-2-[5-({3-[1-(oxiran-2-ylmethyl)-4-{[4-(pyrrolidin-1-yl)piperidin-1- |
| | yl]methyl}-1H-indol-2-yl]prop-2-yn-1-yl}amino)pyridin-2-yl]propanenitrile |
| 167. | 2-(5-{[3-(3-ethyl-6-{[(oxan-4-yl)amino]methyl}-1H-indol-2-yl)prop-2-yn-1- |
| | yl]amino}pyridin-2-yl)-2-methylpropanenitrile |
| 168. | 2-methyl-2-(5-{[3-(6-{[(oxan-4-yl)amino]methyl}-3-(2,2,2-trifluoroethyl)-1H- |
| | indol-2-yl)prop-2-yn-1-yl]amino}pyridin-2-yl)propanenitrile |
| 169. | 2-(5-{[3-(1-acetyl-3-ethyl-6-{[(oxan-4-yl)amino]methyl}-1H-indol-2-yl)prop-2- |
| | yn-1-yl]amino}pyridin-2-yl)-2-methylpropanenitrile |
| 170. | 2-(5-{[3-(3-ethyl-6-{[(1-methylpiperidin-4-yl)amino]methyl}-1H-indol-2-yl)prop- |
| | 2-yn-1-yl]amino}pyridin-2-yl)-2-methylpropanenitrile |
| 171. | 2-methyl-2-(5-{[3-(6-{[(1-methylpiperidin-4-yl)amino]methyl}-3-(2,2,2- |
| | trifluoroethyl)-1H-indol-2-yl)prop-2-yn-1-yl]amino}pyridin-2-yl)propanenitrile |
| 172. | 2-{5-[(3-{6-chloro-4-[(1-methylpiperidin-4-yl)amino]-1-(2,2,2-trifluoroethyl)-1H- |
| | indol-2-yl}prop-2-yn-1-yl)amino]pyridin-2-yl}-2-methylpropanenitrile |
| 173. | 2-(3-{[6-(1-cyano-1-methylethyl)pyridin-3-yl]amino}prop-1-yn-1-yl)-6-fluoro-N- |
| | (1-methylpiperidin-4-yl)-1-(2,2,2-trifluoroethyl)-1H-indole-4-carboxamide |
| 174. | 2-[5-({3-[6-fluoro-4-(4-methylpiperazine-1-carbonyl)-1-(2,2,2-trifluoroethyl)-1H- |
| | indol-2-yl]prop-2-yn-1-yl}amino)pyridin-2-yl]-2-methylpropanenitrile |
| 175. | 6-fluoro-2-{3-[(6-methanesulfonylpyridin-3-yl)amino]prop-1-yn-1-yl}-N-(1- |
| | methylpiperidin-4-yl)-1-(2,2,2-trifluoroethyl)-1H-indol-4-amine |
| 176. | 2-{5-[(3-{6-fluoro-4-[(1-methylpiperidin-4-yl)amino]-1-(2,2,2-trifluoroethyl)-1H- |
| | indol-2-yl}prop-2-yn-1-yl)amino]pyridin-2-yl}-2-methylpropanenitrile |
| 177. | 5-[(3-{6-fluoro-4-[(1-methylpiperidin-4-yl)amino]-1-(2,2,2-trifluoroethyl)-1H- |
| | indol-2-yl}prop-2-yn-1-yl)amino]-N-(pyridin-3-yl)pyridine-2-carboxamide |
| 178. | 2-methyl-2-(5-{[3-(5-{[(oxan-4-yl)amino]methyl}-1-(2,2,2-trifluoroethyl)-1H- |
| | pyrrolo[2,3-b]pyridin-2-yl)prop-2-yn-1-yl]amino}pyridin-2-yl)propanenitrile |
| 179. | 2-methyl-2-{5-[(3-{4-[(1-methylpiperidin-4-yl)amino]-1-(2,2,2-trifluoroethyl)- |
| | 1H-pyrrolo[2,3-b]pyridin-2-yl}prop-2-yn-1-yl)amino]pyridin-2-yl}propanenitrile |
| 180. | 2-(5-{[3-(7-chloro-1-ethyl-4-{[4-(pyrrolidin-1-yl)piperidin-1-yl]methyl}-1H- |
| | indol-2-yl)prop-2-yn-1-yl]amino}pyridin-2-yl)-2-methylpropanenitrile |
| 181. | 2-(5-{[3-(7-chloro-5-{[(oxan-4-yl)amino]methyl}-1-(2,2,2-trifluoroethyl)-1H- |
| | indol-2-yl)prop-2-yn-1-yl]amino}pyridin-2-yl)-2-methylpropanenitrile |
| 182. | 2-(5-{[3-(7-chloro-5-{[(1-methylpiperidin-4-yl)amino]methyl}-1-(2,2,2- |
| | trifluoroethyl)-1H-indol-2-yl)prop-2-yn-1-yl]amino}pyridin-2-yl)-2- |
| | methylpropanenitrile |
| 183. | 2-{5-[(3-{7-fluoro-4-[(1-methylpiperidin-4-yl)amino]-1-(2,2,2-trifluoroethyl)-1H- |
| | indol-2-yl}prop-2-yn-1-yl)amino]pyridin-2-yl}-2-methylpropanenitrile |
| 184. | 2-(5-{[3-(7-fluoro-5-{[(oxan-4-yl)amino]methyl}-1-(2,2,2-trifluoroethyl)-1H- |
| | indol-2-yl)prop-2-yn-1-yl]amino}pyridin-2-yl)-2-methylpropanenitrile |
| 185. | 2-(5-{[3-(7-fluoro-5-{[(1-methylpiperidin-4-yl)amino]methyl}-1-(2,2,2- |
| | trifluoroethyl)-1H-indol-2-yl)prop-2-yn-1-yl]amino}pyridin-2-yl)-2- |
| | methylpropanenitrile |
| 186. | 2-methyl-2-(5-{[3-(5-{[(oxan-4-yl)amino]methyl}-1-(2,2,2-trifluoroethyl)-1H-1,3- |
| | benzodiazol-2-yl)prop-2-yn-1-yl]amino}pyridin-2-yl)propanenitrile |
| 187. | N-{[2-(2-phenylethynyl)-1-(2,2,2-trifluoroethyl)-1H-1,3-benzodiazol-5- |
| | yl]methyl}oxan-4-amine |
| 188. | 2-methyl-2-(5-{[3-(5-{[(oxan-4-yl)amino]methyl}-1-(2,2,2-trifluoroethyl)-1H- |
| | pyrrolo[3,2-b]pyridin-2-yl)prop-2-yn-1-yl]amino}pyridin-2-yl)propanenitrile |
| 189. | 2-{3-[(4-methanesulfonylphenyl)amino]prop-1-yn-1-yl}-5-methyl-N-(1- |
| | methylpiperidin-4-yl)-1-(2,2,2-trifluoroethyl)-1H-indol-4-amine |
| 190. | 4-[(2-{3-[(4-methanesulfonylphenyl)amino]prop-1-yn-1-yl}-5-methyl-1-(2,2,2- |
| | trifluoroethyl)-1H-indol-4-yl)amino]-1λ⁶-thiane-1,1-dione |
| 191. | 2-methyl-2-{5-[(3-{5-methyl-4-[(1-methylpiperidin-4-yl)amino]-1-(2,2,2- |
| | trifluoroethyl)-1H-indol-2-yl}prop-2-yn-1-yl)amino]pyridin-2-yl}propanenitrile |
| 192. | N-[1-(2-methanesulfonylethyl)piperidin-4-yl]-2-{3-[(4- |
| | methanesulfonylphenyl)amino]prop-1-yn-1-yl}-5-methyl-1-(2,2,2-trifluoroethyl)- |
| | 1H-indol-4-amine |
| 193. | 4-[(3-{5-methyl-4-[(piperidin-4-yl)amino]-1-(2,2,2-trifluoroethyl)-1H-indol-2- |
| | yl}prop-2-yn-1-yl)amino]benzene-1-sulfonamide |
| 194. | 2-{3-[(4-methanesulfonylphenyl)amino]prop-1-yn-1-yl}-5-methyl-N-[1-(oxan-4- |
| | yl)piperidin-4-yl]-1-(2,2,2-trifluoroethyl)-1H-indol-4-amine |
| 195 | 2-{4-[(2-{3-[(4-methanesulfonylphenyl)amino]prop-1-yn-1-yl}-5-methyl-1-(2,2,2- |
| | trifluoroethyl)-1H-indol-4-yl)amino]piperidin-1-yl}ethan-1-ol |
| 196. | 2-[5-({3-[4-(methoxymethyl)-1-(2,2,2-trifluoroethyl)-1H-indol-2-yl]prop-2-yn-1- |
| | yl}amino)pyridin-2-yl]-2-methylpropanenitrile |
| 197. | 2-[5-({3-[4-(cyanomethyl)-1-(2,2,2-trifluoroethyl)-1H-indol-2-yl]prop-2-yn-1- |
| | yl}amino)pyridin-2-yl]-2-methylpropanenitrile |
| 198. | 2-methyl-2-[5-({3-[5-(morpholine-4-carbonyl)-1-(2,2,2-trifluoroethyl)-1H-indol- |
| | 2-yl]prop-2-yn-1-yl}amino)pyridin-2-yl]propanenitrile |
| 199. | 2-methyl-2-[5-({3-[5-(4-methylpiperazine-1-carbonyl)-1-(2,2,2-trifluoroethyl)- |
| | 1H-indol-2-yl]prop-2-yn-1-yl}amino)pyridin-2-yl]propanenitrile |
| 200. | 2-{5-[(3-{5-[4-(dimethylamino)piperidine-1-carbonyl]-1-(2,2,2-trifluoroethyl)- |
| | 1H-indol-2-yl}prop-2-yn-1-yl)amino]pyridin-2-yl}-2-methylpropanenitrile |
| 201. | 2-(3-{[6-(1-cyano-1-methylethyl)pyridin-3-yl]amino}prop-1-yn-1-yl)-N-{1-[2- |
| | (dimethylamino)acetyl]piperidin-4-yl}-1-(2,2,2-trifluoroethyl)-1H-indole-5- |
| | carboxamide |
| 202. | 2-(3-{[6-(1-cyano-1-methylethyl)pyridin-3-yl]amino}prop-1-yn-1-yl)-N-(oxan-4- |
| | yl)-1-(2,2,2-trifluoroethyl)-1H-indole-5-carboxamide |
| 203. | 2-methyl-2-(5-{[3-(5-{1-[(oxan-4-yl)amino]ethyl}-1-(2,2,2-trifluoroethyl)-1H- |
| | indol-2-yl)prop-2-yn-1-yl]amino}pyridin-2-yl)propanenitrile |
| 204. | 2-methyl-2-{5-[(3-{4-[(1-methylpiperidin-4-yl)amino]-1-(2,2,2-trifluoroethyl)- |
| | 1H-pyrrolo[3,2-c]pyridin-2-yl}prop-2-yn-1-yl)amino]pyridin-2-yl}propanenitrile |
| 205. | 2-methyl-2-[5-({3-[5-(morpholin-4-ylmethyl)-1-(2,2,2-trifluoroethyl)-1H-indol-2- |
| | yl]prop-2-yn-1-yl}amino)pyridin-2-yl]propanenitrile |
| 206. | 2-[5-({3-[5-({[1-(2-cyanoethyl)-piperidin-4-yl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 207. | 2-methyl-2-(5-{[3-(5-{[(1-methylazetidin-3-yl)amino]methyl}-1-(2,2,2- |
| | trifluoroethyl)-1H-indol-2-yl)prop-2-yn-1-yl]amino}pyridin-2-yl)propanenitrile |
| 208. | 2-methyl-2-(5-{[3-(5-{[(oxetan-3-yl)amino]methyl}-1-(2,2,2-trifluoroethyl)-1H- |
| | indol-2-yl)prop-2-yn-1-yl]amino}pyridin-2-yl)propanenitrile |
| 209. | 2-(5-{[3-(5-{[4-(dimethylamino)-piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 210. | 2-methyl-2-{5-[(3-{5-[({1-[2-(4-methylpiperazin-1-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}propanenitrile |
| 211. | 2-(5-{[3-(5-{[(1-methoxypropan-2-yl)amino]methyl}-1-(2,2,2-trifluoroethyl)-1H- |
| | indol-2-yl)prop-2-yn-1-yl]amino}pyridin-2-yl)-2-methylpropanenitrile |
| 212. | 2-methyl-2-(5-{[3-(5-{[(pyridin-4-ylmethyl)amino]methyl}-1-(2,2,2- |
| | trifluoroethyl)-1H-indol-2-yl)prop-2-yn-1-yl]amino}pyridin-2-yl)propanenitrile |
| 213. | 2-methyl-2-(5-{[3-(5-{[(pyridin-3-ylmethyl)amino]methyl}-1-(2,2,2- |
| | trifluoroethyl)-1H-indol-2-yl)prop-2-yn-1-yl]amino}pyridin-2-yl)propanenitrile |
| 214. | 2-[5-({3-[5-({[1-(dimethylamino)-propan-2-yl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 215. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N-(oxan-4- |
| | yl)acetamide |
| 216. | 2-[5-({3-[5-({[1-(2-methoxyacetyl)-piperidin-4-yl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 217. | 2-methyl-2-{5-[(3-{5-[({1-[2-(oxan-4-yl)acetyl]piperidin-4-yl}amino)-methyl]-1- |
| | (2,2,2-trifluoroethyl)-1H-indol-2-yl}prop-2-yn-1-yl)amino]-pyridin-2- |
| | yl}propanenitrile |
| 218. | 2-methyl-2-{5-[(3-{5-[({1-[2-(pyridin-3-yl)acetyl]piperidin-4-yl}amino)-methyl]- |
| | 1-(2,2,2-trifluoroethyl)-1H-indol-2-yl}prop-2-yn-1-yl)amino]-pyridin-2- |
| | yl}propanenitrile |
| 219. | 2-methyl-2-(5-{[3-(5-{[(1-{2-[(oxan-4-yl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)-propanenitrile |
| 220. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N-methyl-N- |
| | (propan-2-yl)acetamide |
| 221. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N-(2- |
| | methoxyethyl)-N-methylacetamide |
| 222. | 6-methanesulfonyl-N-[3-(5-{[(oxan-4-yl)amino]methyl}-1-(2,2,2-trifluoroethyl)- |
| | 1H-indol-2-yl)prop-2-yn-1-yl]pyridin-3-amine |
| 223. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N,N- |
| | dimethylacetamide |
| 224. | 2-methyl-2-{5-[(3-{5-[({1-[2-oxo-2-(pyrrolidin-1-yl)ethyl]piperidin-4- |
| | yl}amino)methyl]-1-(2,2,2-trifluoroethyl)-1H-indol-2-yl}prop-2-yn-1- |
| | yl)amino]pyridin-2-yl}propanenitrile |
| 225. | 4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)-N,N-dimethylpiperidine-1- |
| | carboxamide |
| 226. | 2-{5-[(3-{5-[({1-[2-(azetidin-1-yl)-2-oxoethyl]piperidin-4-yl}amino)-methyl]-1- |
| | (2,2,2-trifluoroethyl)-1H-indol-2-yl}prop-2-yn-1-yl)amino]-pyridin-2-yl}-2- |
| | methylpropanenitrile |
| 227. | 2-methyl-2-{5-[(3-{5-[({1-[2-(pyrrolidin-1-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}-propanenitrile |
| 228. | 2-(5-{[3-(5-{[(1-{2-[4-(dimethylamino)piperidin-1-yl]acetyl}piperidin-4- |
| | yl)amino]-methyl}-1-(2,2,2-trifluoroethyl)-1H-indol-2-yl)prop-2-yn-1- |
| | yl]amino}pyridin-2-yl)-2-methylpropanenitrile |
| 229. | 2-{5-[(3-{5-[({1-[2-(diethylamino)acetyl]piperidin-4-yl}amino)methyl]-1-(2,2,2- |
| | trifluoroethyl)-1H-indol-2-yl}prop-2-yn-1-yl)amino]pyridin-2-yl}-2- |
| | methylpropanenitrile |
| 230. | 2-methyl-2-(5-{[3-(5-{[(1-{2-[methyl(propan-2-yl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)propanenitrile |
| 231. | 2-methyl-2-{5-[(3-{5-[({1-[2-(pyridin-4-yl)acetyl]piperidin-4-yl}amino)methyl]- |
| | 1-(2,2,2-trifluoroethyl)-1H-indol-2-yl}prop-2-yn-1-yl)amino]pyridin-2- |
| | yl}propanenitrile |
| 232. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N-(pyridin-4- |
| | yl)acetamide |
| 233. | 2-methyl-2-{5-[(3-{5-[({1-[2-(morpholin-4-yl)-2-oxoethyl]piperidin-4- |
| | yl}amino)methyl]-1-(2,2,2-trifluoroethyl)-1H-indol-2-yl}prop-2-yn-1- |
| | yl)amino]pyridin-2-yl}propanenitrile |
| 234. | 2-methyl-2-{5-[(3-{5-[({1-[2-(4-methylpiperazin-1-yl)-2-oxoethyl]piperidin-4- |
| | yl}amino)methyl]-1-(2,2,2-trifluoroethyl)-1H-indol-2-yl}prop-2-yn-1- |
| | yl)amino]pyridin-2-yl}propanenitrile |
| 235. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N-(pyridin-3- |
| | yl)acetamide |
| 236. | 2-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)piperidin-1-yl]-N-(1- |
| | methylpiperidin-4-yl)acetamide |
| 237. | 2-methyl-2-[5-({3-[5-({[4-(morpholin-4-yl)cyclohexyl]amino}methyl)-1-(2,2,2- |
| | trifluoroethyl)-1H-indol-2-yl]prop-2-yn-1-yl}amino)pyridin-2-yl]propanenitrile |
| 238. | 2-{5-[(3-{5-[({1-[2-(4-hydroxypiperidin-1-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}-2-methylpropanenitrile |
| 239. | 2-{5-[(3-{5-[({1-[2-(4-acetylpiperazin-1-yl)acetyl]piperidin-4-yl}amino)methyl]- |
| | 1-(2,2,2-trifluoroethyl)-1H-indol-2-yl}prop-2-yn-1-yl)amino]pyridin-2-yl}-2- |
| | methylpropanenitrile |
| 240. | 2-(5-{[3-(5-{[(1,1-dioxo-1λ⁶-thian-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 241. | 2-{5-[(3-{5-[({1-[2-(1,1-dioxo-1λ⁶,4-thiomorpholin-4-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}-2-methylpropanenitrile |
| 242. | 2-[5-({3-[5-({[1-(4-acetylpiperazine-1-carbonyl)piperidin-4-yl]amino}methyl)-1- |
| | (2,2,2-trifluoroethyl)-1H-indol-2-yl]prop-2-yn-1-yl}amino)pyridin-2-yl]-2- |
| | methylpropanenitrile |
| 243. | 2-(5-{[3-(5-{[(1-{2-[bis(2-hydroxyethyl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)-2-methylpropanenitrile |
| 244. | 2-methyl-2-{5-[(3-{5-[({1-[2-(3-oxopiperazin-1-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}propanenitrile |
| 245. | 2-methyl-2-[5-({3-[5-({[1-(morpholine-4-carbonyl)piperidin-4-yl]amino}- |
| | methyl)-1-(2,2,2-trifluoroethyl)-1H-indol-2-yl]prop-2-yn-1-yl}amino)pyridin-2- |
| | yl]propanenitrile |
| 246. | 2-methyl-2-(5-{[3-(5-{[(1-methylpiperidin-3-yl)amino]methyl}-1-(2,2,2- |
| | trifluoroethyl)-1H-indol-2-yl)prop-2-yn-1-yl]amino}pyridin-2-yl)propanenitrile |
| 247. | N-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)-cyclohexyl]acetamide |
| 248. | 2-{5-[(3-{5-[({1-[2-(1H-imidazol-1-yl)acetyl]piperidin-4-yl}amino)-methyl]-1- |
| | (2,2,2-trifluoroethyl)-1H-indol-2-yl}prop-2-yn-1-yl)amino]pyridin-2-yl}-2- |
| | methylpropanenitrile |
| 249. | 2-(5-{[3-(5-{[(1-{2-[(2-methoxyethyl)(methyl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)-2-methylpropanenitrile |
| 250. | N-[4-({[2-(3-{[6-(1-cyano-1-methylethyl)pyridin-3-yl]amino}prop-1-yn-1-yl)-1- |
| | (2,2,2-trifluoroethyl)-1H-indol-5-yl]methyl}amino)- |
| | cyclohexyl]methanesulfonamide |
| 251. | 2-methyl-2-(5-{[3-(5-{[(1-methyl-6-oxopiperidin-3-yl)amino]methyl}-1-(2,2,2- |
| | trifluoroethyl)-1H-indol-2-yl)prop-2-yn-1-yl]amino}pyridin-2-yl)propanenitrile |
| 252. | 2-[5-({3-[5-({[3-(dimethylamino)cyclohexyl]amino}methyl)-1-(2,2,2- |
| | trifluoroethyl)-1H-indol-2-yl]prop-2-yn-1-yl}amino)pyridin-2-yl]-2- |
| | methylpropanenitrile |
| 253. | 2-methyl-2-[5-({3-[5-({[1-(4-methylpiperazine-1-carbonyl)piperidin-4- |
| | yl]amino}methyl)-1-(2,2,2-trifluoroethyl)-1H-indol-2-yl]prop-2-yn-1- |
| | yl}amino)pyridin-2-yl]propanenitrile |
| 254. | 2-{5-[(3-{5-[({1-[4-(dimethylamino)piperidine-1-carbonyl]piperidin-4-yl}amino)- |
| | methyl]-1-(2,2,2-trifluoroethyl)-1H-indol-2-yl}prop-2-yn-1-yl)amino]pyridin-2- |
| | yl}-2-methylpropanenitrile |
| 255. | 2-{5-[(3-{5-[({1-[2-(3-hydroxypyrrolidin-1-yl)acetyl]-piperidin-4- |
| | yl}amino)methyl]-1-(2,2,2-trifluoroethyl)-1H-indol-2-yl}prop-2-yn-1- |
| | yl)amino]pyridin-2-yl}-2-methylpropanenitrile |
| 256. | 2-{5-[(3-{5-[({1-[2-(3-methoxypyrrolidin-1-yl)acetyl]-piperidin-4- |
| | yl}amino)methyl]-1-(2,2,2-trifluoroethyl)-1H-indol-2-yl}prop-2-yn-1- |
| | yl)amino]pyridin-2-yl}-2-methylpropanenitrile |
| 257. | 2-methyl-2-[5-({3-[5-({[1-(2-{2-oxa-8-azaspiro[4.5]decan-8-yl}acetyl)-piperidin- |
| | 4-yl]amino}methyl)-1-(2,2,2-trifluoroethyl)-1H-indol-2-yl]prop-2-yn-1- |
| | yl}amino)pyridin-2-yl]propanenitrile |
| 258. | 2-{5-[(3-{5-[({1-[2-(4-hydroxy-4-methylpiperidin-1-yl)acetyl]piperidin-4- |
| | yl}amino)methyl]-1-(2,2,2-trifluoroethyl)-1H-indol-2-yl}prop-2-yn-1- |
| | yl)amino]pyridin-2-yl}-2-methylpropanenitrile |
| 259. | 2-(5-{[3-(5-{[(1-{2-[bis(2-methoxyethyl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)-2-methylpropanenitrile |
| 260. | 2-(5-{[3-(5-{[(1-{2-[methoxy(methyl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)-2-methylpropanenitrile |
| 261. | 2-(5-{[3-(5-{[(1-{2-[(2,3-dihydroxypropyl)(methyl)amino]acetyl}piperidin-4- |
| | yl)amino]methyl}-1-(2,2,2-trifluoroethyl)-1H-indol-2-yl)prop-2-yn-1- |
| | yl]amino}pyridin-2-yl)-2-methylpropanenitrile |
| 262. | 2-methyl-2-(5-{[3-(5-{[(1-methyl-2-oxopiperidin-4-yl)amino]methyl}-1-(2,2,2- |
| | trifluoroethyl)-1H-indol-2-yl)prop-2-yn-1-yl]amino}pyridin-2-yl)propanenitrile |
| 263. | 2-methyl-2-(5-{[3-(5-{[(1-{2-[methyl(1-methylpiperidin-4- |
| | yl)amino]acetyl}piperidin-4-yl)amino]methyl}-1-(2,2,2-trifluoroethyl)-1H-indol- |
| | 2-yl)prop-2-yn-1-yl]amino}pyridin-2-yl)propanenitrile |
| 264. | 2-methyl-2-[5-({3-[5-({[1-(2-{9-methyl-3,9-diazaspiro[5.5]undecan-3- |
| | yl}acetyl)piperidin-4-yl]amino}methyl)-1-(2,2,2-trifluoroethyl)-1H-indol-2- |
| | yl]prop-2-yn-1-yl}amino)pyridin-2-yl]propanenitrile |
| 265. | 2-(5-{[3-(5-{[(1-{2-[3-(dimethyl-amino)pyrrolidin-1-yl]acetyl}piperidin-4- |
| | yl)amino]methyl}-1-(2,2,2-trifluoroethyl)-1H-indol-2-yl)prop-2-yn-1- |
| | yl]amino}pyridin-2-yl)-2-methylpropanenitrile |
| 266. | N-[3-(5-{[(oxan-4-yl)amino]methyl}-1-(2,2,2-trifluoroethyl)-1H-indol-2-yl)prop- |
| | 2-yn-1-yl]-6-(pyrrolidine-1-carbonyl)pyridin-3-amine |
| 267. | 6-(morpholine-4-carbonyl)-N-[3-(5-{[(oxan-4-yl)amino]methyl}-1-(2,2,2- |
| | trifluoroethyl)-1H-indol-2-yl)prop-2-yn-1-yl]pyridin-3-amine |
| 268. | 2-chloro-N-[3-(5-{[(oxan-4-yl)amino]-methyl}-1-(2,2,2-trifluoroethyl)-1H-indol- |
| | 2-yl)prop-2-yn-1-yl]pyrimidin-5-amine |
| 269. | 5-{[3-(5-{[(oxan-4-yl)amino]methyl}-1-(2,2,2-trifluoroethyl)-1H-indol-2-yl)prop- |
| | 2-yn-1-yl]amino}-N-phenylpyridine-2-carboxamide |
| 270. | N-methyl-5-{[3-(5-{[(oxan-4-yl)amino]methyl}-1-(2,2,2-trifluoroethyl)-1H-indol- |
| | 2-yl)prop-2-yn-1-yl]amino}-N-(propan-2-yl)pyridine-2-carboxamide |
| 271. | 5-{[3-(5-{[(oxan-4-yl)amino]methyl}-1-(2,2,2-trifluoroethyl)-1H-indol-2-yl)prop- |
| | 2-yn-1-yl]amino}-N-(pyridin-4-yl)pyridine-2-carboxamide |
| 272. | 5-{[3-(5-{[(oxan-4-yl)amino]methyl}-1-(2,2,2-trifluoroethyl)-1H-indol-2-yl)prop- |
| | 2-yn-1-yl]amino}-N-(pyridin-3-yl)pyridine-2-carboxamide |
| 273. | N-(1-methylazetidin-3-yl)-5-{[3-(5-{[(oxan-4-yl)amino]methyl}-1-(2,2,2- |
| | trifluoroethyl)-1H-indol-2-yl)prop-2-yn-1-yl]amino}pyridine-2-carboxamide |
| 274. | N,N-diethyl-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 275. | 5-{[3-(5-{[(oxan-4-yl)amino]methyl}-1-(2,2,2-trifluoroethyl)-1H-indol-2-yl)prop- |
| | 2-yn-1-yl]amino}-N-(oxetan-3-yl)pyridine-2-carboxamide |
| 276. | 1-(4-{[(2-{3-[(2-tert-butylpyrimidin-5-yl)amino]prop-1-yn-1-yl}-1-(2,2,2- |
| | trifluoroethyl)-1H-indol-5-yl)methyl]amino}piperidin-1-yl)-2- |
| | (dimethylamino)ethan-1-one |
| 277. | 1-(4-{[(2-{3-[(6-chloropyridin-3-yl)amino]prop-1-yn-1-yl}-1-(2,2,2- |
| | trifluoroethyl)-1H-indol-5-yl)methyl]amino}piperidin-1-yl)-2- |
| | (dimethylamino)ethan-1-one |
| 278. | 5-[(3-{5-[({1-[2-(dimethylamino)-acetyl]piperidin-4-yl}amino)methyl]-1-(2,2,2- |
| | trifluoroethyl)-1H-indol-2-yl}prop-2-yn-1-yl)amino]-N-(1-methylpiperidin-4- |
| | yl)pyridine-2-carboxamide |
| 279. | 1-(4-{[(2-{3-[(4-chloro-3-fluorophenyl)amino]prop-1-yn-1-yl}-1-(2,2,2- |
| | trifluoroethyl)-1H-indol-5-yl)methyl]amino}piperidin-1-yl)-2- |
| | (dimethylamino)ethan-1-one |
| 280. | 2-(dimethylamino)-1-(4-{[(2-{3-[(6-methylpyridin-3-yl)amino]prop-1-yn-1-yl}-1- |
| | (2,2,2-trifluoroethyl)-1H-indol-5-yl)methyl]amino}piperidin-1-yl)ethan-1-one |
| 281. | 1-(4-{[(2-{3-[(6-tert-butylpyridin-3-yl)amino]prop-1-yn-1-yl}-1-(2,2,2- |
| | trifluoroethyl)-1H-indol-5-yl)methyl]amino}piperidin-1-yl)-2- |
| | (dimethylamino)ethan-1-one |
| 282. | N-[6-(1-cyano-1-methylethyl)pyridin-3-yl]-3-[1-(2-fluoroethyl)-5-{[(1- |
| | methylpiperidin-4-yl)amino]methyl}-1H-indol-2-yl]prop-2-ynamide |
| 283. | 2-{5-[(3-{5-[({1-[2-(dimethylamino)acetyl]piperidin-4-yl}amino)methyl]-1-ethyl- |
| | 1H-indol-2-yl}prop-2-yn-1-yl)amino]pyridin-2-yl}-2-methylpropanenitrile |
| 284. | 2-[5-({3-[1-(2,2-difluoroethyl)-5-[({1-[2-(dimethylamino)acetyl]piperidin-4- |
| | yl}amino)methyl]-1H-indol-2-yl]prop-2-yn-1-yl}amino)pyridin-2-yl]-2- |
| | methylpropanenitrile |
| 285. | 2-{5-[(3-{5-[({1-[2-(dimethylamino)acetyl]piperidin-4-yl}amino)methyl]-1-(2- |
| | fluoroethyl)-1H-indol-2-yl}prop-2-yn-1-yl)amino]pyridin-2-yl}-2- |
| | methylpropanenitrile |
| 286. | N-(6-chloropyridin-3-yl)-3-(5-{[(1-methylpiperidin-4-yl)amino]methyl}-1-(2,2,2- |
| | trifluoroethyl)-1H-indol-2-yl)prop-2-ynamide |
| 287. | 2-(5-{[3-(5-{[(1-acetylpiperidin-4-yl)amino]methyl}-1-(oxiran-2-ylmethyl)-1H- |
| | indol-2-yl)prop-2-yn-1-yl]amino}pyridin-2-yl)-2-methylpropanenitrile |
| 288. | 2-methyl-2-{5-[(3-{5-[(oxan-4-yl)amino]-1-(2,2,2-trifluoroethyl)-1H-indol-2- |
| | yl}prop-2-yn-1-yl)amino]pyridin-2-yl}propanenitrile |
| 289. | 2-{5-[(3-{4-[(1-acetylpiperidin-4-yl)amino]-1-(2,2,2-trifluoroethyl)-1H-indol-2- |
| | yl}prop-2-yn-1-yl)amino]pyridin-2-yl}-2-methylpropanenitrile |
| 290. | 2-methyl-2-{5-[(3-{4-[(propan-2-yl)amino]-1-(2,2,2-trifluoroethyl)-1H-indol-2- |
| | yl}prop-2-yn-1-yl)amino]pyridin-2-yl}propanenitrile |
| 291. | 2-methyl-2-{5-[(3-{4-[(piperidin-4-yl)amino]-1-(2,2,2-trifluoroethyl)-1H-indol-2- |
| | yl}prop-2-yn-1-yl)amino]pyridin-2-yl}propanenitrile |
| 292. | 2-(5-{[3-(4-{[1-(2-methoxyethyl)-piperidin-4-yl]amino}-1-(2,2,2-trifluoroethyl)- |
| | 1H-indol-2-yl)prop-2-yn-1-yl]amino}pyridin-2-yl)-2-methylpropanenitrile |
| 293. | 2-{5-[(3-{4-[(1,1-dioxo-1λ⁶-thian-4-yl)amino]-1-(2,2,2-trifluoroethyl)-1H-indol-2- |
| | yl}prop-2-yn-1-yl)amino]pyridin-2-yl}-2-methylpropanenitrile |
| 294. | 3-[2-(3-{[6-(1-cyano-1-methylethyl)-pyridin-3-yl]amino}prop-1-yn-1-yl)-1-(2,2,2- |
| | trifluoroethyl)-1H-indol-4-yl]-1-(oxan-4-yl)urea |
| 295. | 3-[2-(3-{[6-(1-cyano-1-methylethyl)-pyridin-3-yl]amino}prop-1-yn-1-yl)-1-(2,2,2- |
| | trifluoroethyl)-1H-indol-4-yl]-1-(1-methylpiperidin-4-yl)urea |
| 296. | 2-(4-{[2-(3-{[6-(1-cyano-1-methylethyl)-pyridin-3-yl]amino}prop-1-yn-1-yl)-1- |
| | (2,2,2-trifluoroethyl)-1H-indol-4-yl]amino}piperidin-1-yl)-N,N- |
| | dimethylacetamide |
| 297. | 2-methyl-2-(5-{[3-(4-{[1-(propan-2-yl)piperidin-4-yl]amino}-1-(2,2,2- |
| | trifluoroethyl)-1H-indol-2-yl)prop-2-yn-1-yl]amino}pyridin-2-yl)propanenitrile |
| 298. | 2-methyl-2-(5-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}pyridin-2-yl)propanenitrile |
| 299. | 4-{[2-(3-{[6-(1-cyano-1-methylethyl)-pyridin-3-yl]amino}prop-1-yn-1-yl)-1- |
| | (2,2,2-trifluoroethyl)-1H-indol-4-yl]amino}-N,N-dimethylpiperidine-1- |
| | carboxamide |
| 300. | N-[2-(3-{[6-(1-cyano-1-methylethyl)-pyridin-3-yl]amino}prop-1-yn-1-yl)-1- |
| | (2,2,2-trifluoroethyl)-1H-indol-4-yl]-4-methylpiperazine-1-carboxamide |
| 301. | 1-[2-(3-{[6-(1-cyano-1-methylethyl)pyridin-3-yl]amino}prop-1-yn-1-yl)-1-(2,2,2- |
| | trifluoroethyl)-1H-indol-4-yl]-3,3-dimethylurea |
| 302. | N-[2-(3-{[6-(1-cyano-1-methylethyl)-pyridin-3-yl]amino}prop-1-yn-1-yl)-1- |
| | (2,2,2-trifluoroethyl)-1H-indol-4-yl]morpholine-4-carboxamide |
| 303. | 2-{5-[(3-{4-[(4-hydroxycyclohexyl)-amino]-1-(2,2,2-trifluoroethyl)-1H-indol-2- |
| | yl}prop-2-yn-1-yl)amino]pyridin-2-yl}-2-methylpropanenitrile |
| 304. | 2-methyl-2-[5-({3-[4-({1-[2-(4-methylpiperazin-1-yl)-2-oxoethyl]-piperidin-4- |
| | yl}amino)-1-(2,2,2-trifluoroethyl)-1H-indol-2-yl]prop-2-yn-1-yl}amino)pyridin-2- |
| | yl]propanenitrile |
| 305. | 2-methyl-2-{5-[(3-{4-[(oxan-4-ylmethyl)amino]-1-(2,2,2-trifluoroethyl)-1H-indol- |
| | 2-yl}prop-2-yn-1-yl)amino]pyridin-2-yl}propanenitrile |
| 306. | 2-{5-[(3-{4-[(1-ethylpiperidin-4-yl)amino]-1-(2,2,2-trifluoroethyl)-1H-indol-2- |
| | yl}prop-2-yn-1-yl)amino]pyridin-2-yl}-2-methylpropanenitrile |
| 307. | 2-(5-{[3-(4-{[1-(2-hydroxyethyl)piperidin-4-yl]amino}-1-(2,2,2-trifluoroethyl)- |
| | 1H-indol-2-yl)prop-2-yn-1-yl]amino}pyridin-2-yl)-2-methylpropanenitrile |
| 308. | 2-{5-[(3-{4-[(1-methanesulfonylpiperidin-4-yl)amino]-1-(2,2,2-trifluoroethyl)-1H- |
| | indol-2-yl}prop-2-yn-1-yl)amino]pyridin-2-yl}-2-methylpropanenitrile |
| 309. | 2-(5-{[3-(4-{[1-(2-methanesulfonyl-ethyl)piperidin-4-yl]amino}-1-(2,2,2- |
| | trifluoroethyl)-1H-indol-2-yl)prop-2-yn-1-yl]amino}pyridin-2-yl)-2- |
| | methylpropanenitrile |
| 310. | 2-methyl-2-(5-{[3-(4-{[(1R,4R)-4-hydroxycyclohexyl]amino}-1-(2,2,2- |
| | trifluoroethyl)-1H-indol-2-yl)prop-2-yn-1-yl]amino}pyridin-2-yl)propanenitrile |
| 311. | 2-methyl-2-(5-{[3-(4-{[(1S,4S)-4-hydroxycyclohexyl]amino}-1-(2,2,2- |
| | trifluoroethyl)-1H-indol-2-yl)prop-2-yn-1-yl]amino}pyridin-2-yl)propanenitrile |
| 312. | 2-methyl-2-[5-({3-[4-({1-[2-(morpholin-4-yl)-2-oxoethyl]piperidin-4-yl}amino)- |
| | 1-(2,2,2-trifluoroethyl)-1H-indol-2-yl]prop-2-yn-1-yl}amino)pyridin-2- |
| | yl]propanenitrile |
| 313. | 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]amino}piperidin-1-yl)-N,N-bis(2- |
| | methoxyethyl)acetamide |
| 314. | 2-methyl-2-{5-[(3-{4-[(pyrrolidin-3-yl)amino]-1-(2,2,2-trifluoroethyl)-1H-indol- |
| | 2-yl}prop-2-yn-1-yl)amino]pyridin-2-yl}propanenitrile |
| 315. | 2-methyl-2-{5-[(3-{4-[(1-methylpyrrolidin-3-yl)amino]-1-(2,2,2-trifluoroethyl)- |
| | 1H-indol-2-yl}prop-2-yn-1-yl)amino]pyridin-2-yl}propanenitrile |
| 316. | 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]amino}piperidin-1-yl)acetamide |
| 317. | methyl 2-(4-{[2-(3-{[6-(1-cyano-1-methylethyl)pyridin-3-yl]amino}prop-1-yn-1- |
| | yl)-1-(2,2,2-trifluoroethyl)-1H-indol-4-yl]amino}piperidin-1-yl)acetate |
| 318. | 2-[5-({3-[4-({1-[2-(4-hydroxypiperidin-1-yl)-2-oxoethyl]piperidin-4-yl}amino)-1- |
| | (2,2,2-trifluoroethyl)-1H-indol-2-yl]prop-2-yn-1-yl}amino)pyridin-2-yl]-2- |
| | methylpropanenitrile |
| 319. | 2-methyl-2-{5-[(3-{4-[(2-methylpiperidin-4-yl)amino]-1-(2,2,2-trifluoroethyl)- |
| | 1H-indol-2-yl}prop-2-yn-1-yl)amino]pyridin-2-yl}propanenitrile |
| 320. | 2-{5-[(3-{4-[(1,1-dioxo-1λ⁶-thiolan-3-yl)amino]-1-(2,2,2-trifluoroethyl)-1H-indol- |
| | 2-yl}prop-2-yn-1-yl)amino]pyridin-2-yl}-2-methylpropanenitrile |
| 321. | 2-methyl-2-[5-({3-[4-({1-[2-oxo-2-(pyrrolidin-1-yl)ethyl]piperidin-4-yl}amino)-1- |
| | (2,2,2-trifluoroethyl)-1H-indol-2-yl]prop-2-yn-1-yl}amino)pyridin-2- |
| | yl]propanenitrile |
| 322. | 2-{5-[(3-{4-[(1-{2-[4-(dimethylamino)piperidin-1-yl]-2-oxoethyl}piperidin-4- |
| | yl)amino]-1-(2,2,2-trifluoroethyl)-1H-indol-2-yl}prop-2-yn-1-yl)amino]pyridin-2- |
| | yl}-2-methylpropanenitrile |
| 323. | 2-[5-({3-[4-({1-[2-(dimethylamino)acetyl]piperidin-4-yl}amino)-1-(2,2,2- |
| | trifluoroethyl)-1H-indol-2-yl]prop-2-yn-1-yl}amino)pyridin-2-yl]-2- |
| | methylpropanenitrile |
| 324. | 2-(5-{[3-(4-{[1-(1,1-dioxo-1λ⁶-thian-4-yl)piperidin-4-yl]amino}-1-(2,2,2- |
| | trifluoroethyl)-1H-indol-2-yl)prop-2-yn-1-yl]amino}pyridin-2-yl)-2- |
| | methylpropanenitrile |
| 325. | 2-(5-{[3-(4-{[1-(cyanomethyl)piperidin-4-yl]amino}-1-(2,2,2-trifluoroethyl)-1H- |
| | indol-2-yl)prop-2-yn-1-yl]amino}pyridin-2-yl)-2-methylpropanenitrile |
| 326. | 2-methyl-2-[5-({3-[1-(2,2,2-trifluoroethyl)-4-{[1-(2,2,2-trifluoroethyl)piperidin-4- |
| | yl]amino}-1H-indol-2-yl]prop-2-yn-1-yl}amino)pyridin-2-yl]propanenitrile |
| 327. | 2-{5-[(3-{4-[(1-{2-[4-(2-methanesulfonylethyl)piperazin-1-yl]-2- |
| | oxoethyl}piperidin-4-yl)amino]-1-(2,2,2-trifluoroethyl)-1H-indol-2-yl}prop-2-yn- |
| | 1-yl)amino]pyridin-2-yl}-2-methylpropanenitrile |
| 328. | 2-[5-({3-[4-({1-[2-(1,1-dioxo-1λ⁶,4-thiomorpholin-4-yl)-2-oxoethyl]piperidin-4- |
| | yl}amino)-1-(2,2,2-trifluoroethyl)-1H-indol-2-yl]prop-2-yn-1-yl}amino)pyridin-2- |
| | yl]-2-methylpropanenitrile |
| 329. | 2-(5-{[3-(4-{[1-(1-methanesulfonylpiperidin-4-yl)piperidin-4-yl]amino}-1-(2,2,2- |
| | trifluoroethyl)-1H-indol-2-yl)prop-2-yn-1-yl]amino}pyridin-2-yl)-2- |
| | methylpropanenitrile |
| 330. | 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]amino}piperidin-1-yl)-N-(2,3- |
| | dihydroxypropyl)-N-methylacetamide |
| 331. | 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]amino}piperidin-1-yl)-N-(2,3- |
| | dihydroxypropyl)acetamide |
| 332. | 2-[5-({3-[4-({1-[2-(4-methanesulfonylpiperazin-1-yl)-2-oxoethyl]piperidin-4- |
| | yl}amino)-1-(2,2,2-trifluoroethyl)-1H-indol-2-yl]prop-2-yn-1-yl}amino)pyridin-2- |
| | yl]-2-methylpropanenitrile |
| 333. | 2-{5-[(3-{4-[(1-{2-[4-(2-hydroxyethyl)piperazin-1-yl]-2-oxoethyl}piperidin-4- |
| | yl)amino]-1-(2,2,2-trifluoroethyl)-1H-indol-2-yl}prop-2-yn-1-yl)amino]pyridin-2- |
| | yl}-2-methylpropanenitrile |
| 334. | 2-methyl-2-(5-{[3-(4-{[1-(oxan-4-yl)piperidin-4-yl]amino}-1-(2,2,2- |
| | trifluoroethyl)-1H-indol-2-yl)prop-2-yn-1-yl]amino}pyridin-2-yl)propanenitrile |
| 335. | 2-[5-({3-[4-({1-[1-(2-methanesulfonylethyl)piperidin-4-yl]piperidin-4-yl}amino)- |
| | 1-(2,2,2-trifluoroethyl)-1H-indol-2-yl]prop-2-yn-1-yl}amino)pyridin-2-yl]-2- |
| | methylpropanenitrile |
| 336. | 2-[5-({3-[4-({1-[1-(2-methoxyethyl)piperidin-4-yl]piperidin-4-yl}amino)-1-(2,2,2- |
| | trifluoroethyl)-1H-indol-2-yl]prop-2-yn-1-yl}amino)pyridin-2-yl]-2- |
| | methylpropanenitrile |
| 337. | 2-[5-({3-[4-({1-[1-(2-hydroxyethyl)piperidin-4-yl]piperidin-4-yl}amino)-1-(2,2,2- |
| | trifluoroethyl)-1H-indol-2-yl]prop-2-yn-1-yl}amino)pyridin-2-yl]-2- |
| | methylpropanenitrile |
| 338. | 2-[5-({3-[4-({1-[2-(dimethylamino)ethyl]piperidin-4-yl}amino)-1-(2,2,2- |
| | trifluoroethyl)-1H-indol-2-yl]prop-2-yn-1-yl}amino)pyridin-2-yl]-2- |
| | methylpropanenitrile |
| 339. | 2-(5-{[3-(4-{[1-(1-acetylpiperidin-4-yl)piperidin-4-yl]amino}-1-(2,2,2- |
| | trifluoroethyl)-1H-indol-2-yl)prop-2-yn-1-yl]amino}pyridin-2-yl)-2- |
| | methylpropanenitrile |
| 340. | 2-methyl-2-[5-({3-[4-({1-[(1R,4R)-4-hydroxycyclohexyl]piperidin-4-yl}amino)-1- |
| | (2,2,2-trifluoroethyl)-1H-indol-2-yl]prop-2-yn-1-yl}amino)pyridin-2- |
| | yl]propanenitrile |
| 341. | 2-methyl-2-[5-({3-[4-({1-[(1S,4S)-4-hydroxycyclohexyl]piperidin-4-yl}amino)-1- |
| | (2,2,2-trifluoroethyl)-1H-indol-2-yl]prop-2-yn-1-yl}amino)pyridin-2- |
| | yl]propanenitrile |
| 342. | N-(1-methylpiperidin-4-yl)-2-{3-[(6-methylpyridin-3-yl)amino]prop-1-yn-1-yl}-1- |
| | (2,2,2-trifluoroethyl)-1H-indol-4-amine |
| 343. | 2-methyl-2-{5-[(3-{4-[(1-methylpiperidin-4-yl)amino]-1-(2,2,2-trifluoroethyl)- |
| | 1H-indol-2-yl}prop-2-yn-1-yl)amino]pyridin-2-yl}propanenitrile |
| 344. | 5-[(3-{4-[(1-methylpiperidin-4-yl)amino]-1-(2,2,2-trifluoroethyl)-1H-indol-2- |
| | yl}prop-2-yn-1-yl)amino]-N-(pyridin-3-yl)pyridine-2-carboxamide |
| 345. | 5-[(3-{4-[(1-methylpiperidin-4-yl)amino]-1-(2,2,2-trifluoroethyl)-1H-indol-2- |
| | yl}prop-2-yn-1-yl)amino]pyridine-2-carbonitrile |
| 346. | N-[6-(1-cyano-1-methylethyl)pyridin-3-yl]-3-{4-[(1-methylpiperidin-4-yl)amino]- |
| | 1-(2,2,2-trifluoroethyl)-1H-indol-2-yl}prop-2-ynamide |
| 347. | 2-{3-[(2-fluorophenyl)amino]prop-1-yn-1-yl}-N-(1-methylpiperidin-4-yl)-1- |
| | (2,2,2-trifluoroethyl)-1H-indol-4-amine |
| 348. | 2-{3-[(3-fluorophenyl)amino]prop-1-yn-1-yl}-N-(1-methylpiperidin-4-yl)-1- |
| | (2,2,2-trifluoroethyl)-1H-indol-4-amine |
| 349. | 4-amino-N-(3-{4-[(1-methylpiperidin-4-yl)amino]-1-(2,2,2-trifluoroethyl)-1H- |
| | indol-2-yl}prop-2-yn-1-yl)benzene-1-sulfonamide |
| 350. | 2-{3-[(6-tert-butylpyridin-3-yl)amino]prop-1-yn-1-yl}-N-(1-methylpiperidin-4- |
| | yl)-1-(2,2,2-trifluoroethyl)-1H-indol-4-amine |
| 351. | 2-{3-[(4-fluorophenyl)amino]prop-1-yn-1-yl}-N-(1-methylpiperidin-4-yl)-1- |
| | (2,2,2-trifluoroethyl)-1H-indol-4-amine |
| 352. | N,N-dimethyl-5-[(3-{4-[(1-methylpiperidin-4-yl)amino]-1-(2,2,2-trifluoroethyl)- |
| | 1H-indol-2-yl}prop-2-yn-1-yl)amino]pyridine-2-carboxamide |
| 353. | 5-[(3-{4-[(1-methylpiperidin-4-yl)amino]-1-(2,2,2-trifluoroethyl)-1H-indol-2- |
| | yl}prop-2-yn-1-yl)amino]-N-(propan-2-yl)pyridine-2-carboxamide |
| 354. | N-(pyridin-3-yl)-5-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}pyridine-2- |
| | carboxamide |
| 355. | N-(pyridin-3-yl)-5-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}pyridine-2- |
| | carboxamide |
| 356. | 2-{3-[(6-methanesulfonylpyridin-3-yl)amino]prop-1-yn-1-yl}-N-(1- |
| | methylpiperidin-4-yl)-1-(2,2,2-trifluoroethyl)-1H-indol-4-amine |
| 357. | 6-tert-butyl-N-(3-{4-[(1-methylpiperidin-4-yl)amino]-1-(2,2,2-trifluoroethyl)-1H- |
| | indol-2-yl}prop-2-yn-1-yl)pyridine-3-carboxamide |
| 358. | 2-{3-[(6-chloropyridin-3-yl)amino]prop-1-yn-1-yl}-N-(1-methylpiperidin-4-yl)-1- |
| | (2,2,2-trifluoroethyl)-1H-indol-4-amine |
| 359. | 2-{4-[(3-{4-[(1-methylpiperidin-4-yl)amino]-1-(2,2,2-trifluoroethyl)-1H-indol-2- |
| | yl}prop-2-yn-1-yl)amino]phenyl}propan-2-ol |
| 360. | 6-methyl-N-(3-{4-[(1-methylpiperidin-4-yl)amino]-1-(2,2,2-trifluoroethyl)-1H- |
| | indol-2-yl}prop-2-yn-1-yl)pyridine-3-carboxamide |
| 361. | N-(1-methylpiperidin-4-yl)-1-(2,2,2-trifluoroethyl)-2-(3-{[6- |
| | (trifluoromethyl)pyridin-3-yl]amino}prop-1-yn-1-yl)-1H-indol-4-amine |
| 362. | 3-(3-{4-[(1-methylpiperidin-4-yl)amino]-1-(2,2,2-trifluoroethyl)-1H-indol-2- |
| | yl}prop-2-yn-1-yl)-1-phenylurea |
| 363. | 2-{3-[(4-tert-butyl-2-fluorophenyl)-amino]prop-1-yn-1-yl}-N-(1-methylpiperidin- |
| | 4-yl)-1-(2,2,2-trifluoroethyl)-1H-indol-4-amine |
| 364. | 2-{3-fluoro-4-[(3-{4-[(1-methylpiperidin-4-yl)amino]-1-(2,2,2-trifluoroethyl)-1H- |
| | indol-2-yl}prop-2-yn-1-yl)amino]phenyl}-2-methylpropanenitrile |
| 365. | 4-[(3-{4-[(1-methylpiperidin-4-yl)amino]-1-(2,2,2-trifluoroethyl)-1H-indol-2- |
| | yl}prop-2-yn-1-yl)amino]benzene-1-sulfonamide |
| 366. | 2-{3-[(2,6-difluoro-4-methane-sulfonylphenyl)amino]prop-1-yn-1-yl}-N-(1- |
| | methylpiperidin-4-yl)-1-(2,2,2-trifluoroethyl)-1H-indol-4-amine |
| 367. | N-methyl-4-[(3-{4-[(1-methylpiperidin-4-yl)amino]-1-(2,2,2-trifluoroethyl)-1H- |
| | indol-2-yl}prop-2-yn-1-yl)amino]benzene-1-sulfonamide |
| 368. | 2-{3-[(4-methanesulfonyl-3-methoxyphenyl)amino]prop-1-yn-1-yl}-N-(1- |
| | methylpiperidin-4-yl)-1-(2,2,2-trifluoroethyl)-1H-indol-4-amine |
| 369. | 2-{3-[(4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 370. | 2-{3-[(5-methanesulfonylpyridin-2-yl)amino]prop-1-yn-1-yl}-N-(1- |
| | methylpiperidin-4-yl)-1-(2,2,2-trifluoroethyl)-1H-indol-4-amine |
| 371. | 2-{3-[(4-methanesulfonyl-2-methylphenyl)amino]prop-1-yn-1-yl}-N-(1- |
| | methylpiperidin-4-yl)-1-(2,2,2-trifluoroethyl)-1H-indol-4-amine |
| 372. | methyl 3-methoxy-4-[(3-{4-[(1-methylpiperidin-4-yl)amino]-1-(2,2,2- |
| | trifluoroethyl)-1H-indol-2-yl}prop-2-yn-1-yl)amino]benzoate |
| 373. | N-{3-methoxy-4-[(3-{4-[(1-methylpiperidin-4-yl)amino]-1-(2,2,2-trifluoroethyl)- |
| | 1H-indol-2-yl}prop-2-yn-1-yl)amino]phenyl}methanesulfonamide |
| 374. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]benzonitrile |
| 375. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]benzoic acid |
| 376. | 2-{3-[(2,4-dimethoxyphenyl)amino]prop-1-yn-1-yl}-N-(1-methylpiperidin-4-yl)- |
| | 1-(2,2,2-trifluoroethyl)-1H-indol-4-amine |
| 377. | 2-{3-[(2-methoxypyridin-3-yl)amino]prop-1-yn-1-yl}-N-(1-methylpiperidin-4-yl)- |
| | 1-(2,2,2-trifluoroethyl)-1H-indol-4-amine |
| 378. | 2-{3-[(5-fluoro-2-methoxyphenyl)-amino]prop-1-yn-1-yl}-N-(1-methylpiperidin- |
| | 4-yl)-1-(2,2,2-trifluoroethyl)-1H-indol-4-amine |
| 379. | 2-{3-[(2-ethoxy-4-methane-sulfonylphenyl)amino]prop-1-yn-1-yl}-N-(1- |
| | methylpiperidin-4-yl)-1-(2,2,2-trifluoroethyl)-1H-indol-4-amine |
| 380. | 2-{3-[(3-fluoro-2-methoxyphenyl)-amino]prop-1-yn-1-yl}-N-(1-methylpiperidin- |
| | 4-yl)-1-(2,2,2-trifluoroethyl)-1H-indol-4-amine |
| 381. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]benzene-1-sulfonamide |
| 382. | 2-{3-[(4-fluoro-2-methoxyphenyl)-amino]prop-1-yn-1-yl}-N-(1-methylpiperidin- |
| | 4-yl)-1-(2,2,2-trifluoroethyl)-1H-indol-4-amine |
| 383. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]-benzamide |
| 384. | 2-{3-[(2-fluoro-6-methoxyphenyl)-amino]prop-1-yn-1-yl}-N-(1-methylpiperidin- |
| | 4-yl)-1-(2,2,2-trifluoroethyl)-1H-indol-4-amine |
| 385. | 2-{3-[(4-tert-butyl-2-methoxyphenyl)-amino]prop-1-yn-1-yl}-N-(1- |
| | methylpiperidin-4-yl)-1-(2,2,2-trifluoroethyl)-1H-indol-4-amine |
| 386. | 4-methoxy-3-[(3-{4-[(1-methylpiperidin-4-yl)amino]-1-(2,2,2-trifluoroethyl)-1H- |
| | indol-2-yl}prop-2-yn-1-yl)amino]-benzonitrile |
| 387. | 2-{3-[(5-tert-butyl-2-methoxyphenyl)-amino]prop-1-yn-1-yl}-N-(1- |
| | methylpiperidin-4-yl)-1-(2,2,2-trifluoroethyl)-1H-indol-4-amine |
| 388. | N-(1-methylpiperidin-4-yl)-2-[3-(phenylamino)prop-1-yn-1-yl]-1-(2,2,2- |
| | trifluoroethyl)-1H-indol-4-amine |
| 389. | 5-methanesulfonyl-2-[(3-{4-[(1-methylpiperidin-4-yl)amino]-1-(2,2,2- |
| | trifluoroethyl)-1H-indol-2-yl}prop-2-yn-1-yl)amino]benzonitrile |
| 390. | 2-(3-{[2-(2-fluoroethoxy)-4-methanesulfonylphenyl]amino}prop-1-yn-1-yl)-N-(1- |
| | methylpiperidin-4-yl)-1-(2,2,2-trifluoroethyl)-1H-indol-4-amine |
| 391. | 2-{3-[(3-methoxypyridin-4-yl)amino]prop-1-yn-1-yl}-N-(1-methylpiperidin-4-yl)- |
| | 1-(2,2,2-trifluoroethyl)-1H-indol-4-amine |
| 392. | 2-{3-[(2-chloro-4-methanesulfonyl-phenyl)amino]prop-1-yn-1-yl}-N-(1- |
| | methylpiperidin-4-yl)-1-(2,2,2-trifluoroethyl)-1H-indol-4-amine |
| 393. | 2-{3-[(4-methoxyphenyl)amino]prop-1-yn-1-yl}-N-(1-methylpiperidin-4-yl)-1- |
| | (2,2,2-trifluoroethyl)-1H-indol-4-amine |
| 394. | 2-(3-{[4-methanesulfonyl-2-(2-methoxyethoxy)phenyl]amino}prop-1-yn-1-yl)-N- |
| | (1-methylpiperidin-4-yl)-1-(2,2,2-trifluoroethyl)-1H-indol-4-amine |
| 395. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]pyridine-2-carbonitrile |
| 396. | 4-{[2-(3-{[6-(morpholine-4-carbonyl)pyridin-3-yl]amino}prop-1-yn-1-yl)-1- |
| | (2,2,2-trifluoroethyl)-1H-indol-4-yl]amino}-1λ⁶-thiane-1,1-dione |
| 397. | 4-{[2-(3-{[6-(4-methylpiperazine-1-carbonyl)pyridin-3-yl]amino}prop-1-yn-1-yl)- |
| | 1-(2,2,2-trifluoroethyl)-1H-indol-4-yl]amino}-1λ⁶-thiane-1,1-dione |
| 398. | 4-[(2-{3-[(quinolin-3-yl)amino]prop-1-yn-1-yl}-1-(2,2,2-trifluoroethyl)-1H-indol- |
| | 4-yl)amino]-1λ⁶-thiane-1,1-dione |
| 399. | 4-[(2-{3-[(quinoxalin-6-yl)amino]prop-1-yn-1-yl}-1-(2,2,2-trifluoroethyl)-1H- |
| | indol-4-yl)amino]-1λ⁶-thiane-1,1-dione |
| 400. | 4-[(2-{3-[(4-methanesulfonylphenyl)-amino]prop-1-yn-1-yl}-1-(2,2,2- |
| | trifluoroethyl)-1H-indol-4-yl)amino]-1λ⁶-thiane-1,1-dione |
| 401. | 5-[(3-{4-[(1, 1-dioxo-1λ⁶-thian-4-yl)amino]-1-(2,2,2-trifluoroethyl)-1H-indol-2- |
| | yl}prop-2-yn-1-yl)amino]pyridine-2-carboxamide |
| 402. | 4-[(2-{3-[(6-methoxypyridin-3-yl)amino]prop-1-yn-1-yl}-1-(2,2,2-trifluoroethyl)- |
| | 1H-indol-4-yl)amino]-1λ⁶-thiane-1,1-dione |
| 403. | 4-{[2-(3-{[6-(4-hydroxypiperidine-1-carbonyl)pyridin-3-yl]amino}prop-1-yn-1- |
| | yl)-1-(2,2,2-trifluoroethyl)-1H-indol-4-yl]amino}-1λ⁶-thiane-1,1-dione |
| 404. | 4-[(2-{3-[(1-methyl-2-oxo-1,2-dihydropyridin-4-yl)amino]prop-1-yn-1-yl}-1- |
| | (2,2,2-trifluoroethyl)-1H-indol-4-yl)amino]-1λ⁶-thiane-1,1-dione |
| 405. | 4-[(2-{3-[(2-methoxypyridin-4-yl)amino]prop-1-yn-1-yl}-1-(2,2,2-trifluoroethyl)- |
| | 1H-indol-4-yl)amino]-1λ⁶-thiane-1,1-dione |
| 406. | 2-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-fluorophenyl}-2-methylpropanenitrile |
| 407. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-(1-methylpiperidin-4-yl)pyridine-2-carboxamide |
| 408. | 4-[(2-{3-[(2-fluoro-4-methanesulfonyl-phenyl)amino]prop-1-yn-1-yl}-1-(2,2,2- |
| | trifluoroethyl)-1H-indol-4-yl)amino]-1λ⁶-thiane-1,1-dione |
| 409. | 4-{[2-(3-{[4-(ethanesulfonyl)-phenyl]amino}prop-1-yn-1-yl)-1-(2,2,2- |
| | trifluoroethyl)-1H-indol-4-yl]amino}-1λ⁶-thiane-1,1-dione |
| 410. | 4-[(2-{3-[(2-tert-butylpyrimidin-5-yl)amino]prop-1-yn-1-yl}-1-(2,2,2- |
| | trifluoroethyl)-1H-indol-4-yl)amino]-1λ⁶-thiane-1,1-dione |
| 411. | 3-{4-[(1,1-dioxo-1λ⁶-thian-4-yl)amino]-1-(2,2,2-trifluoroethyl)-1H-indol-2-yl}-N- |
| | (4-methanesulfonyl-phenyl)-prop-2-ynamide |
| 412. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-(oxan-4-yl)pyridine-2-carboxamide |
| 413. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-(pyridin-3-yl)pyridine-2-carboxamide |
| 414. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-methylpyridine-2-carboxamide |
| 415. | 4-[(2-{3-[(3-chloro-4-methanesulfonylphenyl)amino]prop-1-yn-1-yl}-1-(2,2,2- |
| | trifluoroethyl)-1H-indol-4-yl)amino]-1λ⁶-thiane-1,1-dione |
| 416. | N-(2,3-dihydroxypropyl)-5-[(3-{4-[(1,1-dioxo-1λ⁶-thian-4-yl)amino]-1-(2,2,2- |
| | trifluoroethyl)-1H-indol-2-yl}prop-2-yn-1-yl)amino]pyridine-2-carboxamide |
| 417. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-hydroxypyridine-2-carboxamide |
| 418. | 5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-(2-hydroxyethyl)pyridine-2-carboxamide |
| 419. | 5-[(3-{4-[(1, 1-dioxo-1λ⁶-thian-4-yl)amino]-1-(2,2,2-trifluoroethyl)-1H-indol-2- |
| | yl}prop-2-yn-1-yl)amino]-N-hydroxy-N-methylpyridine-2-carboxamide |
| 420. | 4-amino-N-(3-{4-[(1,1-dioxo-1λ⁶-thian-4-yl)amino]-1-(2,2,2-trifluoroethyl)-1H- |
| | indol-2-yl}prop-2-yn-1-yl)benzene-1-sulfonamide |
| 421. | 4-({2-[3-({pyrido[2,3-b]pyrazin-7-yl}amino)prop-1-yn-1-yl]-1-(2,2,2- |
| | trifluoroethyl)-1H-indol-4-yl}amino)-1λ⁶-thiane-1,1-dione |
| 422. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-benzamide |
| 423. | 4-{[2-(3-{[2-(methylsulfanyl)pyrimidin-5-yl]amino}prop-1-yn-1-yl)-1-(2,2,2- |
| | trifluoroethyl)-1H-indol-4-yl]amino}-1λ⁶-thiane-1,1-dione |
| 424. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]benzene-1-sulfonamide |
| 425. | 4-{[2-(3-{[4-(2-methylpropane-2-sulfonyl)phenyl]amino}prop-1-yn-1-yl)-1- |
| | (2,2,2-trifluoroethyl)-1H-indol-4-yl]amino}-1λ⁶-thiane-1,1-dione |
| 426. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N,N-dimethylbenzene-1-sulfonamide |
| 427. | 4-{[1-(2,2,2-trifluoroethyl)-2-[2-(trimethylsilyl)ethynyl]-1H-indol-4-yl]amino}- |
| | 1λ⁶-thiane-1,1-dione |
| 428. | 4-[(2-{3-[(5-methanesulfonylpyridin-2-yl)amino]prop-1-yn-1-yl}-1-(2,2,2- |
| | trifluoroethyl)-1H-indol-4-yl)amino]-1λ⁶-thiane-1,1-dione |
| 429. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]-1λ⁶-thiane-1,1-dione |
| 430. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-methylbenzene-1-sulfonamide |
| 431. | 4-{[2-ethynyl-1-(2,2,2-trifluoroethyl)-1H-indol-4-yl]amino}-1λ⁶-thiane-1,1-dione |
| 432. | N-{4-[(3-{4-[(1,1-dioxo-1λ⁶-thian-4-yl)amino]-1-(2,2,2-trifluoroethyl)-1H-indol- |
| | 2-yl}prop-2-yn-1-yl)amino]-3-methoxyphenyl}methanesulfonamide |
| 433. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxybenzoic acid |
| 434. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxybenzonitrile |
| 435. | 4-[(2-{3-[(5-fluoro-2-methoxyphenyl)amino]prop-1-yn-1-yl}-1-(2,2,2- |
| | trifluoroethyl)-1H-indol-4-yl)amino]-1λ⁶-thiane-1,1-dione |
| 436. | 4-[(2-{3-[(2-methoxy-6-methylpyridin-3-yl)amino]prop-1-yn-1-yl}-1-(2,2,2- |
| | trifluoroethyl)-1H-indol-4-yl)amino]-1λ⁶-thiane-1,1-dione |
| 437. | 4-[(2-{3-[(2-hydroxy-6-methylpyridin-3-yl)amino]prop-1-yn-1-yl}-1-(2,2,2- |
| | trifluoroethyl)-1H-indol-4-yl)amino]-1λ⁶-thiane-1,1-dione |
| 438. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxybenzamide |
| 439. | 4-[(2-{3-[(2-methoxypyridin-3-yl)amino]prop-1-yn-1-yl}-1-(2,2,2-trifluoroethyl)- |
| | 1H-indol-4-yl)amino]-1λ⁶-thiane-1,1-dione |
| 440. | 4-[(2-{3-[(4-fluoro-2-methoxyphenyl)amino]prop-1-yn-1-yl}-1-(2,2,2- |
| | trifluoroethyl)-1H-indol-4-yl)amino]-1λ⁶-thiane-1,1-dione |
| 441. | 4-[(2-{3-[(5-tert-butyl-2-methoxyphenyl)amino]prop-1-yn-1-yl}-1-(2,2,2- |
| | trifluoroethyl)-1H-indol-4-yl)amino]-1λ⁶-thiane-1,1-dione |
| 442. | 4-[(2-{3-[(2-ethoxy-4-methanesulfonylphenyl)amino]prop-1-yn-1-yl}-1-(2,2,2- |
| | trifluoroethyl)-1H-indol-4-yl)amino]-1λ⁶-thiane-1,1-dione |
| 443. | 4-[(2-{3-[(3-fluoro-2-methoxyphenyl)-amino]prop-1-yn-1-yl}-1-(2,2,2- |
| | trifluoroethyl)-1H-indol-4-yl)amino]-1λ⁶-thiane-1,1-dione |
| 444. | 4-({2-[3-(methylamino)prop-1-yn-1-yl]-1-(2,2,2-trifluoroethyl)-1H-indol-4- |
| | yl}amino)-1λ⁶-thiane-1,1-dione |
| 445. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxybenzene-1-sulfonamide |
| 446. | 4-[(2-{3-[(2-fluoro-6-methoxyphenyl)-amino]prop-1-yn-1-yl}-1-(2,2,2- |
| | trifluoroethyl)-1H-indol-4-yl)amino]-1λ⁶-thiane-1,1-dione |
| 447. | 3-[(3-{4-[(1,1-dioxo-1λ⁶-thian-4-yl)amino]-1-(2,2,2-trifluoroethyl)-1H-indol-2- |
| | yl}prop-2-yn-1-yl)amino]-4-methoxybenzonitrile |
| 448. | 4-[(2-{3-[(4-tert-butyl-2-methoxy-phenyl)amino]prop-1-yn-1-yl}-1-(2,2,2- |
| | trifluoroethyl)-1H-indol-4-yl)amino]-1λ⁶-thiane-1,1-dione |
| 449. | 4-({2-[3-(phenylamino)prop-1-yn-1-yl]-1-(2,2,2-trifluoroethyl)-1H-indol-4- |
| | yl}amino)-1λ⁶-thiane-1,1-dione |
| 450. | 4-{[2-(3-{[2-(2-fluoroethoxy)-4-methanesulfonylphenyl]amino}prop-1-yn-1-yl)- |
| | 1-(2,2,2-trifluoroethyl)-1H-indol-4-yl]amino}-1λ⁶-thiane-1,1-dione |
| 451. | 2-[(3-{4-[(1,1-dioxo-1λ⁶-thian-4-yl)amino]-1-(2,2,2-trifluoroethyl)-1H-indol-2- |
| | yl}prop-2-yn-1-yl)amino]-5-methanesulfonylbenzonitrile |
| 452. | 4-[(2-{3-[(2-chloro-4-methanesulfonyl-phenyl)amino]prop-1-yn-1-yl}-1-(2,2,2- |
| | trifluoroethyl)-1H-indol-4-yl)amino]-1λ⁶-thiane-1,1-dione |
| 453. | 4-[(2-{3-[(4-methoxyphenyl)amino]prop-1-yn-1-yl}-1-(2,2,2-trifluoroethyl)-1H- |
| | indol-4-yl)amino]-1λ⁶-thiane-1,1-dione |
| 454. | 4-{[2-(3-{[4-methanesulfonyl-2-(2-methoxyethoxy)phenyl]amino}prop-1-yn-1- |
| | yl)-1-(2,2,2-trifluoroethyl)-1H-indol-4-yl]amino}-1λ⁶-thiane-1,1-dione |
| 455. | 2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}-N,N-dimethylacetamide |
| 456. | 2-methyl-2-(5-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}pyridin-2-yl)propanenitrile |
| 457. | 2-(5-((3-(4-(((1S,4S)-4-(dimethylamino)-cyclohexyl)amino)-1-(2,2,2- |
| | trifluoroethyl)-1H-indol-2-yl)prop-2-yn-1-yl)amino)pyridin-2-yl)-2- |
| | methylpropanenitrile |
| 458. | N-[1-(2-methanesulfonylethyl)piperidin-4-yl]-2-{3-[(4-methanesulfonylphenyl)- |
| | amino]prop-1-yn-1-yl}-1-(2,2,2-trifluoroethyl)-1H-indol-4-amine |
| 459. | 5-({3-[4-({1-[(dimethylcarbamoyl)methyl]piperidin-4-yl}amino)-1-(2,2,2- |
| | trifluoroethyl)-1H-indol-2-yl]prop-2-yn-1-yl}amino)pyridine-2-carboxamide |
| 460. | 5-{[3-(4-{[1-(2-methanesulfonylethyl)piperidin-4-yl]amino}-1-(2,2,2- |
| | trifluoroethyl)-1H-indol-2-yl)prop-2-yn-1-yl]amino}pyridine-2-carboxamide |
| 461. | 5-[(3-{4-[(piperidin-4-yl)amino]-1-(2,2,2-trifluoroethyl)-1H-indol-2-yl}prop-2-yn- |
| | 1-yl)amino]pyridine-2-carboxamide |
| 462. | 5-{[3-(4-{[1-(carbamoylmethyl)piperidin-4-yl]amino}-1-(2,2,2-trifluoroethyl)-1H- |
| | indol-2-yl)prop-2-yn-1-yl]amino}pyridine-2-carboxamide |
| 463. | 5-{[3-(4-{[1-(2-hydroxyethyl)piperidin-4-yl]amino}-1-(2,2,2-trifluoroethyl)-1H- |
| | indol-2-yl)prop-2-yn-1-yl]amino}pyridine-2-carboxamide |
| 464. | 5-{[3-(4-{[1-(oxan-4-yl)piperidin-4-yl]amino}-1-(2,2,2-trifluoroethyl)-1H-indol- |
| | 2-yl)prop-2-yn-1-yl]amino}pyridine-2-carboxamide |
| 465. | 5-{[3-(4-{[(1R,4R)-4-(dimethylamino)-cyclohexyl]amino}-1-(2,2,2- |
| | trifluoroethyl)-1H-indol-2-yl)prop-2-yn-1-yl]amino}pyridine-2-carboxamide |
| 466. | 4-{[3-(4-{[1-(2-methanesulfonyl-ethyl)piperidin-4-yl]amino}-1-(2,2,2- |
| | trifluoroethyl)-1H-indol-2-yl)prop-2-yn-1-yl]amino}benzene-1-sulfonamide |
| 467. | 4-{[3-(4-{[1-(2-methoxyethyl)piperidin-4-yl]amino}-1-(2,2,2-trifluoroethyl)-1H- |
| | indol-2-yl)prop-2-yn-1-yl]amino}benzene-1-sulfonamide |
| 468. | 5-{[3-(4-{[(1S,4S)-4-(dimethylamino)-cyclohexyl]amino}-1-(2,2,2-trifluoroethyl)- |
| | 1H-indol-2-yl)prop-2-yn-1-yl]amino}pyridine-2-carboxamide |
| 469. | N,N-dimethyl-4-{[3-(4-{[1-(oxan-4-yl)piperidin-4-yl]amino}-1-(2,2,2- |
| | trifluoroethyl)-1H-indol-2-yl)prop-2-yn-1-yl]amino}benzene-1-sulfonamide |
| 470. | 4-{[3-(4-{[1-(oxan-4-yl)piperidin-4-yl]amino}-1-(2,2,2-trifluoroethyl)-1H-indol- |
| | 2-yl)prop-2-yn-1-yl]amino}benzene-1-sulfonamide |
| 471. | 2-{4-[(2-{3-[(4-sulfamoylphenyl)-amino]prop-1-yn-1-yl}-1-(2,2,2-trifluoroethyl)- |
| | 1H-indol-4-yl)amino]-piperidin-1-yl}acetamide |
| 472. | 4-{[3-(4-{[1-(2-methoxyethyl)piperidin-4-yl]amino}-1-(2,2,2-trifluoroethyl)-1H- |
| | indol-2-yl)prop-2-yn-1-yl]amino}-N,N-dimethylbenzene-1-sulfonamide |
| 473. | 4-[(3-{4-[(oxan-4-yl)amino]-1-(2,2,2-trifluoroethyl)-1H-indol-2-yl}prop-2-yn-1- |
| | yl)amino]benzene-1-sulfonamide |
| 474. | 4-{[3-(4-{[1-(2-hydroxypropyl)piperidin-4-yl]amino}-1-(2,2,2-trifluoroethyl)-1H- |
| | indol-2-yl)prop-2-yn-1-yl]amino}benzene-1-sulfonamide |
| 475. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}benzene-1-sulfonamide |
| 476. | 4-({3-[4-({1-[2-(morpholin-4-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 477. | methyl 2-{4-[(2-{3-[(4-sulfamoylphenyl)-amino]prop-1-yn-1-yl}-1-(2,2,2- |
| | trifluoro-ethyl)-1H-indol-4-yl)amino]piperidin-1-yl}acetate |
| 478. | 4-{[3-(4-{[1-(2-hydroxyethyl)piperidin-4-yl]amino}-1-(2,2,2-trifluoroethyl)-1H- |
| | indol-2-yl)prop-2-yn-1-yl]amino}benzene-1-sulfonamide |
| 479. | 4-({3-[4-({1-[2-(2-hydroxyethoxy)ethyl]piperidin-4-yl}amino)-1-(2,2,2- |
| | trifluoroethyl)-1H-indol-2-yl]prop-2-yn-1-yl}amino)benzene-1-sulfonamide |
| 480. | N,N-dimethyl-2-{4-[(2-{3-[(4-sulfamoylphenyl)amino]prop-1-yn-1-yl}-1-(2,2,2- |
| | trifluoroethyl)-1H-indol-4-yl)amino]piperidin-1-yl}acetamide |
| 481. | 4-({3-[4-({1-[2-(4-hydroxypiperidin-1-yl)-2-oxoethyl]piperidin-4-yl}amino)-1- |
| | (2,2,2-trifluoroethyl)-1H-indol-2-yl]prop-2-yn-1-yl}amino)benzene-1-sulfonamide |
| 482. | 2-{4-[(2-{3-[(4-sulfamoylphenyl)amino]prop-1-yn-1-yl}-1-(2,2,2-trifluoroethyl)- |
| | 1H-indol-4-yl)amino]piperidin-1-yl}acetic acid |
| 483. | 4-({3-[4-({1-[2-(4-methylpiperazin-1-yl)-2-oxoethyl]piperidin-4-yl}amino)-1- |
| | (2,2,2-trifluoroethyl)-1H-indol-2-yl]prop-2-yn-1-yl}amino)benzene-1-sulfonamide |
| 484. | N-methyl-2-{4-[(2-{3-[(4-sulfamoylphenyl)amino]prop-1-yn-1-yl}-1-(2,2,2- |
| | trifluoroethyl)-1H-indol-4-yl)amino]piperidin-1-yl}acetamide |
| 485. | N-methyl-4-[(3-{4-[(oxan-4-yl)amino]-1-(2,2,2-trifluoroethyl)-1H-indol-2- |
| | yl}prop-2-yn-1-yl)amino]benzene-1-sulfonamide |
| 486. | 4-{[3-(4-{[1-(2-methoxyethyl)piperidin-4-yl]amino}-1-(2,2,2-trifluoroethyl)-1H- |
| | indol-2-yl)prop-2-yn-1-yl]amino}-N-methylbenzene-1-sulfonamide |
| 487. | N-methyl-4-{[3-(4-{[1-(oxan-4-yl)piperidin-4-yl]amino}-1-(2,2,2-trifluoroethyl)- |
| | 1H-indol-2-yl)prop-2-yn-1-yl]amino}benzene-1-sulfonamide |
| 488. | 2-(dimethylamino)ethyl 2-{4-[(2-{3-[(4-methanesulfonylphenyl)-amino]prop-1- |
| | yn-1-yl}-1-(2,2,2-trifluoroethyl)-1H-indol-4-yl)amino]piperidin-1-yl}acetate |
| 489. | 2-methoxy-4-[(3-{4-[(oxan-4-yl)amino]-1-(2,2,2-trifluoroethyl)-1H-indol-2- |
| | yl}prop-2-yn-1-yl)amino]benzene-1-sulfonamide |
| 490. | 2-chloro-4-[(3-{4-[(oxan-4-yl)amino]-1-(2,2,2-trifluoroethyl)-1H-indol-2-yl}prop- |
| | 2-yn-1-yl)amino]benzene-1-sulfonamide |
| 491. | 3-methoxy-4-{[3-(4-{[1-(oxan-4-yl)piperidin-4-yl]amino}-1-(2,2,2-trifluoroethyl)- |
| | 1H-indol-2-yl)prop-2-yn-1-yl]amino}benzene-1-sulfonamide |
| 492. | 4-{[3-(4-{[1-(2,3-dihydroxypropyl)-piperidin-4-yl]amino}-1-(2,2,2-trifluoro- |
| | ethyl)-1H-indol-2-yl)prop-2-yn-1-yl]amino}-3-methoxybenzene-1-sulfonamide |
| 493. | 3-methoxy-4-[(3-{4-[(piperidin-4-yl)amino]-1-(2,2,2-trifluoroethyl)-1H-indol-2- |
| | yl}prop-2-yn-1-yl)amino]benzene-1-sulfonamide |
| 494. | 3-methoxy-4-[(3-{4-[(oxan-4-yl)amino]-1-(2,2,2-trifluoroethyl)-1H-indol-2- |
| | yl}prop-2-yn-1-yl)amino]benzene-1-sulfonamide |
| 495. | 3-methoxy-4-[(3-{4-[(oxan-4-yl)amino]-1-(2,2,2-trifluoroethyl)-1H-indol-2- |
| | yl}prop-2-yn-1-yl)amino]benzamide |
| 496. | 3-methoxy-4-{[3-(4-{[1-(oxan-4-yl)piperidin-4-yl]amino}-1-(2,2,2-trifluoroethyl)- |
| | 1H-indol-2-yl)prop-2-yn-1-yl]amino}benzamide |
| 497. | 4-{[3-(4-{[1-(2,3-dihydroxypropyl)-piperidin-4-yl]amino}-1-(2,2,2- |
| | trifluoroethyl)-1H-indol-2-yl)prop-2-yn-1-yl]amino}-3-methoxybenzamide |
| 498. | 2-[5-({3-[1-(cyanomethyl)-4-[(1-methylpiperidin-4-yl)amino]-1H-indol-2-yl]prop- |
| | 2-yn-1-yl}amino)pyridin-2-yl]-2-methylpropanenitrile |
| 499. | 2-[5-({3-[1-(3-methoxypropyl)-4-[(1-methylpiperidin-4-yl)amino]-1H-indol-2- |
| | yl]prop-2-yn-1-yl}amino)pyridin-2-yl]-2-methylpropanenitrile |
| 500. | 2-[5-({3-[1-(2-chloroethyl)-4-[(1-methylpiperidin-4-yl)amino]-1H-indol-2- |
| | yl]prop-2-yn-1-yl}amino)pyridin-2-yl]-2-methylpropanenitrile |
| 501. | 2-methyl-2-{5-[(3-{4-[(1-methylpiperidin-4-yl)amino]-1-(propan-2-yl)-1H-indol- |
| | 2-yl}prop-2-yn-1-yl)amino]pyridin-2-yl}propanenitrile |
| 502. | 2-{5-[(3-{1-cyclopentyl-4-[(1-methylpiperidin-4-yl)amino]-1H-indol-2-yl}prop-2- |
| | yn-1-yl)amino]pyridin-2-yl}-2-methylpropanenitrile |
| 503. | 2-methyl-2-{5-[(3-{4-[(1-methylpiperidin-4-yl)amino]-1-(3,3,3-trifluoropropyl)- |
| | 1H-indol-2-yl}prop-2-yn-1-yl)amino]pyridin-2-yl}propanenitrile |
| 504. | 1-(2-chloroethyl)-N-(1-methylpiperidin-4-yl)-2-{3-[(6-methylpyridin-3-yl)amino]- |
| | prop-1-yn-1-yl}-1H-indol-4-amine |
| 505. | 1-(2-chloroethyl)-N-(1-methylpiperidin-4-yl)-2-{3-[(6-methylpyridin-3-yl)amino]- |
| | prop-1-yn-1-yl}-1H-indol-4-amine |
| 506. | 1-(2-chloroethyl)-2-{3-[(4-chlorophenyl)-amino]prop-1-yn-1-yl}-N-(1- |
| | methylpiperidin-4-yl)-1H-indol-4-amine |
| 507. | 2-[5-({3-[1-(1-cyanoethyl)-4-[(1-methylpiperidin-4-yl)amino]-1H-indol-2- |
| | yl]prop-2-yn-1-yl}amino)pyridin-2-yl]-2-methylpropanenitrile |
| 508. | 2-[5-({3-[1-(cyanomethyl)-4-[(1, 1-dioxo-1λ⁶-thian-4-yl)amino]-1H-indol-2- |
| | yl]prop-2-yn-1-yl}amino)pyridin-2-yl]-2-methylpropanenitrile |
| 509. | 4-[(2-{3-[(4-methanesulfonylphenyl)amino]prop-1-yn-1-yl}-1-(oxiran-2- |
| | ylmethyl)-1H-indol-4-yl)amino]-1λ⁶-thiane-1,1-dione |
| 510. | 2-{3-[(4-methanesulfonylphenyl)-amino]prop-1-yn-1-yl}-N-[1-(oxan-4- |
| | yl)piperidin-4-yl]-1-(oxiran-2-ylmethyl)-1H-indol-4-amine |
| 511. | 4-[(2-{3-[(4-methanesulfonyl-2-methoxyphenyl)amino]prop-1-yn-1-yl}-1-(oxiran- |
| | 2-ylmethyl)-1H-indol-4-yl)amino]-1λ⁶-thiane-1,1-dione |
| 512. | 2-{3-[(4-methanesulfonyl-2-methoxy-phenyl)amino]prop-1-yn-1-yl}-N-[1-(oxan- |
| | 4-yl)piperidin-4-yl]-1-(oxiran-2-ylmethyl)-1H-indol-4-amine |
| 513. | 1-[6-(1-cyano-1-methylethyl)pyridin-3-yl]-3-(3-{4-[(1-methylpiperidin-4- |
| | yl)amino]-1-(2,2,2-trifluoroethyl)-1H-indol-2-yl}prop-2-yn-1-yl)urea |
| 514. | 1-(6-methanesulfonylpyridin-3-yl)-3-(3-{4-[(1-methylpiperidin-4-yl)amino]-1- |
| | (2,2,2-trifluoroethyl)-1H-indol-2-yl}prop-2-yn-1-yl)urea |
| 515. | 1-[6-(1-cyano-1-methylethyl)pyridin-3-yl]-3-(3-{4-[(1,1-dioxo-1λ⁶-thian-4- |
| | yl)amino]-1-(2,2,2-trifluoroethyl)-1H-indol-2-yl}prop-2-yn-1-yl)urea |
| 516. | 3-(3-{4-[(1,1-dioxo-1λ⁶-thian-4-yl)amino]-1-(2,2,2-trifluoroethyl)-1H-indol-2- |
| | yl}prop-2-yn-1-yl)-1-(6-methanesulfonylpyridin-3-yl)urea |
| 517. | 1-(6-cyanopyridin-3-yl)-3-(3-{4-[(1-methylpiperidin-4-yl)amino]-1-(2,2,2- |
| | trifluoroethyl)-1H-indol-2-yl}prop-2-yn-1-yl)urea |
| 518. | 1-(6-cyanopyridin-3-yl)-3-(3-{4-[(1,1-dioxo-1λ⁶-thian-4-yl)amino]-1-(2,2,2- |
| | trifluoroethyl)-1H-indol-2-yl}prop-2-yn-1-yl)urea |
| 519. | 3-(3-{4-[(1,1-dioxo-1λ⁶-thian-4-yl)amino]-1-(2,2,2-trifluoroethyl)-1H-indol-2- |
| | yl}prop-2-yn-1-yl)-1-(quinoxalin-6-yl)urea |
| 520. | N-(3-{4-[(1,1-dioxo-1λ⁶-thian-4-yl)amino]-1-(2,2,2-trifluoroethyl)-1H-indol-2- |
| | yl}prop-2-yn-1-yl)-4-methylpiperazine-1-carboxamide |
| 521. | N-(3-{4-[(1,1-dioxo-1λ⁶-thian-4-yl)amino]-1-(2,2,2-trifluoroethyl)-1H-indol-2- |
| | yl}prop-2-yn-1-yl)morpholine-4-carboxamide |
| 522. | 4-[(2-{3-[(6-methanesulfonylpyridin-3-yl)amino]prop-1-yn-1-yl}-1-(2,2,2- |
| | trifluoroethyl)-1H-indol-4-yl)amino]-1λ⁶-thiane-1,1-dione |
| 523. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-N,N-dimethylacetamide |
| 524. | N-(1-ethylpiperidin-4-yl)-2-{3-[(6-methanesulfonylpyridin-3-yl)amino]prop-1-yn- |
| | 1-yl}-1-(2,2,2-trifluoroethyl)-1H-indol-4-amine |
| 525. | N-[1-(2-methanesulfonylethyl)piperidin-4-yl]-2-{3-[(6-methanesulfonylpyridin-3- |
| | yl)amino]prop-1-yn-1-yl}-1-(2,2,2-trifluoroethyl)-1H-indol-4-amine |
| 526. | 2-{3-[(6-methanesulfonylpyridin-3-yl)amino]prop-1-yn-1-yl}-N-[1-(1- |
| | methylpiperidin-4-yl)piperidin-4-yl]-1-(2,2,2-trifluoroethyl)-1H-indol-4-amine |
| 527. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}ethan-1-ol |
| 528. | 4-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-1λ⁶-thiane-1,1-dione |
| 529. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-1-(4-methylpiperazin-1- |
| | yl)ethan-1-one |
| 530. | 2-{3-[(4-methanesulfonylphenyl)amino]prop-1-yn-1-yl}-N-(oxan-4-yl)-1-(2,2,2- |
| | trifluoroethyl)-1H-indol-4-amine |
| 531. | 2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 532. | 2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}-1-(4-methylpiperazin-1- |
| | yl)ethan-1-one |
| 533. | 2-{3-[(4-methanesulfonylphenyl)amino]prop-1-yn-1-yl}-N-[1-(1-methylpiperidin- |
| | 4-yl)piperidin-4-yl]-1-(2,2,2-trifluoroethyl)-1H-indol-4-amine |
| 534. | N-(2,3-dihydroxypropyl)-2-{4-[(2-{3-[(4-methanesulfonylphenyl)-amino]prop-1- |
| | yn-1-yl}-1-(2,2,2-trifluoroethyl)-1H-indol-4-yl)amino]piperidin-1-yl}-N- |
| | methylacetamide |
| 535. | 4-N-(2-{3-[(4-methanesulfonylphenyl)-amino]prop-1-yn-1-yl}-1-(2,2,2- |
| | trifluoroethyl)-1H-indol-4-yl)-1-N,1-N-dimethylcyclohexane-1,4-diamine |
| 536. | (1S,4S)-4-N-(2-{3-[(4-methanesulfonyl-phenyl)amino]prop-1-yn-1-yl}-1-(2,2,2- |
| | trifluoroethyl)-1H-indol-4-yl)-1-N,1-N-dimethylcyclohexane-1,4-diamine |
| 537. | 2-{3-[(4-methanesulfonylphenyl)-amino]prop-1-yn-1-yl}-N-[1-(oxan-4- |
| | yl)piperidin-4-yl]-1-(2,2,2-trifluoroethyl)-1H-indol-4-amine |
| 538. | 2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}-1-(morpholin-4-yl)ethan-1- |
| | one |
| 539. | 1-(4-hydroxypiperidin-1-yl)-2-{4-[(2-{3-[(4-methanesulfonylphenyl)-amino]prop- |
| | 1-yn-1-yl}-1-(2,2,2-trifluoroethyl)-1H-indol-4-yl)amino]piperidin-1-yl}ethan-1- |
| | one |
| 540. | 2-{3-[(4-methanesulfonylphenyl)-amino]prop-1-yn-1-yl}-N-(piperidin-4-yl)-1- |
| | (2,2,2-trifluoroethyl)-1H-indol-4-amine |
| 541. | N-{1-[1-(2-methanesulfonylethyl)piperidin-4-yl]piperidin-4-yl}-2-{3-[(4- |
| | methanesulfonylphenyl)amino]prop-1-yn-1-yl}-1-(2,2,2-trifluoroethyl)-1H-indol- |
| | 4-amine |
| 542. | 2-{3-[(4-methanesulfonylphenyl)-amino]prop-1-yn-1-yl}-N-[1-(2- |
| | methoxyethyl)piperidin-4-yl]-1-(2,2,2-trifluoroethyl)-1H-indol-4-amine |
| 543. | 3-{4-[(2-{3-[(4-methane-sulfonylphenyl)-amino]prop-1-yn-1-yl}-1-(2,2,2- |
| | trifluoroethyl)-1H-indol-4-yl)amino]piperidin-1-yl}propanenitrile |
| 544. | 2-{3-[(4-methanesulfonylphenyl)-amino]prop-1-yn-1-yl}-N-(1-methylpiperidin-4- |
| | yl)-1-(2,2,2-trifluoroethyl)-1H-indol-4-amine |
| 545. | 2-{4-[(2-{3-[(4-methane-sulfonylphenyl)-amino]prop-1-yn-1-yl}-1-(2,2,2- |
| | trifluoroethyl)-1H-indol-4-yl)amino]piperidin-1-yl}acetamide |
| 546. | 4-{4-[(2-{3-[(4-methane-sulfonylphenyl)-amino]prop-1-yn-1-yl}-1-(2,2,2- |
| | trifluoroethyl)-1H-indol-4-yl)amino]piperidin-1-yl}-1λ⁶-thiane-1,1-dione |
| 547. | 2-{4-[(2-{3-[(4-methane-sulfonylphenyl)amino]prop-1-yn-1-yl}-1-(2,2,2- |
| | trifluoroethyl)-1H-indol-4-yl)amino]piperidin-1-yl}-N-methylacetamide |
| 548. | 2-(3-{[4-(ethanesulfonyl)phenyl]amino}prop-1-yn-1-yl)-N-(1-methylpiperidin-4- |
| | yl)-1-(2,2,2-trifluoroethyl)-1H-indol-4-amine |
| 549. | 2-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 550. | 2-{3-[(2-fluoro-4-methane-sulfonylphenyl)-amino]prop-1-yn-1-yl}-N-[1-(2- |
| | methanesulfonylethyl)-piperidin-4-yl]-1-(2,2,2-trifluoroethyl)-1H-indol-4-amine |
| 551. | 2-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}acetamide |
| 552. | 2-{3-[(2-fluoro-4-methane-sulfonylphenyl)-amino]prop-1-yn-1-yl}-N-(1- |
| | methylpiperidin-4-yl)-1-(2,2,2-trifluoroethyl)-1H-indol-4-amine |
| 553. | 1-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}-2-methoxyethan-1-one |
| 554. | 2-{3-[(4-methanesulfonyl-phenyl)amino]prop-1-yn-1-yl}-N-(1-methylpyrrolidin- |
| | 3-yl)-1-(2,2,2-trifluoroethyl)-1H-indol-4-amine |
| 555. | N-hydroxy-2-{4-[(2-{3-[(4-methanesulfonylphenyl)amino]prop-1-yn-1-yl}-1- |
| | (2,2,2-trifluoroethyl)-1H-indol-4-yl)amino]piperidin-1-yl}acetamide |
| 556. | 3-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}propane-1,2-diol |
| 557. | 2-{3-[(2-fluoro-4-methanesulfonyl-phenyl)amino]prop-1-yn-1-yl}-N-[1-(1- |
| | methylpiperidin-4-yl)piperidin-4-yl]-1-(2,2,2-trifluoroethyl)-1H-indol-4-amine |
| 558. | 2-(3-{[4-(ethanesulfonyl)phenyl]amino}prop-1-yn-1-yl)-N-[1-(1-methylpiperidin- |
| | 4-yl)piperidin-4-yl]-1-(2,2,2-trifluoroethyl)-1H-indol-4-amine |
| 559. | 2-{3-[(2-fluoro-4-methanesulfonyl-phenyl)amino]prop-1-yn-1-yl}-N-(oxan-4-yl)- |
| | 1-(2,2,2-trifluoroethyl)-1H-indol-4-amine |
| 560. | 2-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)ethan-1-ol |
| 561. | 1-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}propan-2-ol |
| 562. | 2-(3-{[4-(ethanesulfonyl)-phenyl]amino}prop-1-yn-1-yl)-N-[1-(2- |
| | methanesulfonylethyl)piperidin-4-yl]-1-(2,2,2-trifluoroethyl)-1H-indol-4-amine |
| 563. | 4-{[1-(2,2,2-trifluoroethyl)-2-{3-[(4-trifluoromethanesulfonylphenyl)amino]prop- |
| | 1-yn-1-yl}-1H-indol-4-yl]amino}-1λ⁶-thiane-1,1-dione |
| 564. | 2-(4-{[1-(2,2,2-trifluoroethyl)-2-{3-[(4- |
| | trifluoromethanesulfonylphenyl)amino]prop-1-yn-1-yl}-1H-indol-4- |
| | yl]amino}piperidin-1-yl)ethan-1-ol |
| 565. | N-(1-methylpiperidin-4-yl)-1-(2,2,2-trifluoroethyl)-2-{3-[(4- |
| | trifluoromethanesulfonylphenyl)amino]prop-1-yn-1-yl}-1H-indol-4-amine |
| 566. | 2-{3-[(4-methanesulfonyl-phenyl)amino]prop-1-yn-1-yl}-N-[1-(1- |
| | methylpyrrolidin-3-yl)piperidin-4-yl]-1-(2,2,2-trifluoroethyl)-1H-indol-4-amine |
| 567. | 2-{3-[(4-methanesulfonyl-phenyl)amino]prop-1-yn-1-yl}-N-[1-(3- |
| | methanesulfonylpropyl)piperidin-4-yl]-1-(2,2,2-trifluoroethyl)-1H-indol-4-amine |
| 568. | 2-(3-{[4-(ethanesulfonyl)-phenyl]amino}prop-1-yn-1-yl)-N-[1-(2- |
| | methoxyethyl)piperidin-4-yl]-1-(2,2,2-trifluoroethyl)-1H-indol-4-amine |
| 569. | 4-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)-1λ⁶-thiane-1,1-dione |
| 570. | 2-(3-{[4-(ethanesulfonyl)-phenyl]amino}prop-1-yn-1-yl)-N-[1-(oxan-4- |
| | yl)piperidin-4-yl]-1-(2,2,2-trifluoroethyl)-1H-indol-4-amine |
| 571. | N-[1-(2-methanesulfonylethyl)-piperidin-4-yl]-1-(2,2,2-trifluoroethyl)-2-{3-[(4- |
| | trifluoromethane-sulfonylphenyl)amino]prop-1-yn-1-yl}-1H-indol-4-amine |
| 572. | N-[1-(oxan-4-yl)piperidin-4-yl]-1-(2,2,2-trifluoroethyl)-2-{3-[(4- |
| | trifluoromethanesulfonylphenyl)amino]prop-1-yn-1-yl}-1H-indol-4-amine |
| 573. | N-[1-(1-methylpiperidin-4-yl)piperidin-4-yl]-1-(2,2,2-trifluoroethyl)-2-{3-[(4- |
| | trifluoromethanesulfonylphenyl)amino]prop-1-yn-1-yl}-1H-indol-4-amine |
| 574. | 2-{3-[(2-fluoro-4-methanesulfonyl-phenyl)amino]prop-1-yn-1-yl}-N-[1-(oxan-4- |
| | yl)piperidin-4-yl]-1-(2,2,2-trifluoroethyl)-1H-indol-4-amine |
| 575. | 2-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}acetonitrile |
| 576. | 2-{3-[(2-fluoro-4-methanesulfonylphenyl)amino]prop-1-yn-1-yl}-N-[1-(2- |
| | methoxyethyl)piperidin-4-yl]-1-(2,2,2-trifluoroethyl)-1H-indol-4-amine |
| 577. | 2-{3-[(3-chloro-4-methanesulfonylphenyl)amino]prop-1-yn-1-yl}-N-[1-(oxan-4- |
| | yl)piperidin-4-yl]-1-(2,2,2-trifluoroethyl)-1H-indol-4-amine |
| 578. | 2-{3-[(2,6-difluoro-4-methanesulfonyl-phenyl)amino]prop-1-yn-1-yl}-N-[1- |
| | (oxan-4-yl)piperidin-4-yl]-1-(2,2,2-trifluoroethyl)-1H-indol-4-amine |
| 579. | 2-{3-[(3-chloro-4-methanesulfonyl-phenyl)amino]prop-1-yn-1-yl}-N-[1-(2- |
| | methoxyethyl)piperidin-4-yl]-1-(2,2,2-trifluoroethyl)-1H-indol-4-amine |
| 580. | 2-{4-[(2-{3-[(3-chloro-4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 581. | (2S)-3-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}propane-1,2-diol |
| 582. | N-(5-aminopentyl)-2-{4-[(2-{3-[(4-methanesulfonylphenyl)amino]prop-1-yn-1- |
| | yl}-1-(2,2,2-trifluoroethyl)-1H-indol-4-yl)amino]piperidin-1-yl}acetamide |
| 583. | 2-{3-[(2,6-difluoro-4-methanesulfonyl-phenyl)amino]prop-1-yn-1-yl}-N-[1-(2- |
| | methoxyethyl)piperidin-4-yl]-1-(2,2,2-trifluoroethyl)-1H-indol-4-amine |
| 584. | 2-(3-{[4-(ethanesulfonyl)phenyl]-amino}prop-1-yn-1-yl)-N-(oxan-4-yl)-1-(2,2,2- |
| | trifluoroethyl)-1H-indol-4-amine |
| 585. | 2-(4-{[2-(3-{[4-(ethanesulfonyl)phenyl]amino}prop-1-yn-1-yl)-1-(2,2,2- |
| | trifluoroethyl)-1H-indol-4-yl]amino}piperidin-1-yl)acetonitrile |
| 586. | 2-(3-{[4-(2-methylpropane-2-sulfonyl)phenyl]amino}prop-1-yn-1-yl)-N-[1-(oxan- |
| | 4-yl)piperidin-4-yl]-1-(2,2,2-trifluoroethyl)-1H-indol-4-amine |
| 587. | 2-(2-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}ethoxy)ethan-1-ol |
| 588. | 1-{4-[(2-{3-[(2-fluoro-4-methane-sulfonylphenyl)amino]prop-1-yn-1-yl}-1-(2,2,2- |
| | trifluoroethyl)-1H-indol-4-yl)amino]piperidin-1-yl}propan-2-ol |
| 589. | 3-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}propane-1,2-diol |
| 590. | (1S,4S)-4-N-(2-{3-[(2-fluoro-4-methanesulfonylphenyl)amino]prop-1-yn-1-yl}-1- |
| | (2,2,2-trifluoroethyl)-1H-indol-4-yl)-1-N,1-N-dimethylcyclohexane-1,4-diamine |
| 591. | 3-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)propane-1,2-diol |
| 592. | 2-(3-{[4-(ethanesulfonyl)-phenyl]amino}prop-1-yn-1-yl)-N-[1-(3- |
| | methanesulfonylpropyl)-piperidin-4-yl]-1-(2,2,2-trifluoroethyl)-1H-indol-4-amine |
| 593. | 1-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)propan-2-ol |
| 594. | 2-[2-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)ethoxy]ethan-1-ol |
| 595. | (1R,4R)-4-N-(2-{3-[(2-fluoro-4-methanesulfonylphenyl)amino]prop-1-yn-1-yl}-1- |
| | (2,2,2-trifluoroethyl)-1H-indol-4-yl)-1-N,1-N-dimethyl-cyclohexane-1,4-diamine |
| 596. | 2-{3-[(2,6-difluoro-4-methanesulfonylphenyl)amino]prop-1-yn-1-yl}-N-[1-(2- |
| | methanesulfonylethyl)piperidin-4-yl]-1-(2,2,2-trifluoroethyl)-1H-indol-4-amine |
| 597. | 4-{4-[(2-{3-[(2,6-difluoro-4-methanesulfonylphenyl)amino]prop-1-yn-1-yl}-1- |
| | (2,2,2-trifluoroethyl)-1H-indol-4-yl)amino]piperidin-1-yl}-1λ⁶-thiane-1,1-dione |
| 598. | 2-{3-[(4-methanesulfonyl-3-methoxyphenyl)amino]prop-1-yn-1-yl}-N-[1-(oxan- |
| | 4-yl)piperidin-4-yl]-1-(2,2,2-trifluoroethyl)-1H-indol-4-amine |
| 599. | 2-{3-[(4-methanesulfonyl-3-methoxyphenyl)amino]prop-1-yn-1-yl}-N-[1-(2- |
| | methoxyethyl)piperidin-4-yl]-1-(2,2,2-trifluoroethyl)-1H-indol-4-amine |
| 600. | 4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethylcyclohexane-1,4-diamine |
| 601. | 2-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}acetic acid |
| 602. | 2-hydroxyethyl 2-{4-[(2-{3-[(4-methanesulfonylphenyl)amino]prop-1-yn-1-yl}-1- |
| | (2,2,2-trifluoroethyl)-1H-indol-4-yl)amino]piperidin-1-yl}acetate |
| 603. | 2-{3-[(4-methanesulfonylphenyl)-amino]prop-1-yn-1-yl}-N-(2-methylpiperidin-4- |
| | yl)-1-(2,2,2-trifluoroethyl)-1H-indol-4-amine |
| 604. | 2-{4-[(2-{3-[(4-methanesulfonyl-3-methoxyphenyl)amino]prop-1-yn-1-yl}-1- |
| | (2,2,2-trifluoroethyl)-1H-indol-4-yl)amino]piperidin-1-yl}ethan-1-ol |
| 605. | (2S)-2-(2-{4-[(2-{3-[(4-methane-sulfonylphenyl)amino]prop-1-yn-1-yl}-1-(2,2,2- |
| | trifluoroethyl)-1H-indol-4-yl)amino]piperidin-1-yl}acetamido)pentanedioic acid |
| 606. | 1,5-dimethyl (2S)-2-(2-{4-[(2-{3-[(4-methanesulfonylphenyl)amino]prop-1-yn-1- |
| | yl}-1-(2,2,2-trifluoroethyl)-1H-indol-4-yl)amino]piperidin-1- |
| | yl}acetamido)pentanedioate |
| 607. | N-(4-carbamimidamidobutyl)-2-{4-[(2-{3-[(4-methanesulfonylphenyl)- |
| | amino]prop-1-yn-1-yl}-1-(2,2,2-trifluoroethyl)-1H-indol-4-yl)amino]piperidin-1- |
| | yl}acetamide |
| 608. | 2-{3-[(4-methanesulfonyl-2-methoxyphenyl)amino]prop-1-yn-1-yl}-N-(oxan-4- |
| | yl)-1-(2,2,2-trifluoroethyl)-1H-indol-4-amine |
| 609. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethan-1-ol |
| 610. | 2-{3-[(5-methanesulfonylpyridin-2-yl)amino]prop-1-yn-1-yl}-N-(oxan-4-yl)-1- |
| | (2,2,2-trifluoroethyl)-1H-indol-4-amine |
| 611. | 3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propane-1,2-diol |
| 612. | 4-[(2-{3-[(2,4-dimethoxyphenyl)-amino]prop-1-yn-1-yl}-1-(2,2,2-trifluoroethyl)- |
| | 1H-indol-4-yl)amino]-1λ⁶-thiane-1,1-dione |
| 613. | methyl 4-[(3-{4-[(1,1-dioxo-1λ⁶-thian-4-yl)amino]-1-(2,2,2-trifluoroethyl)-1H- |
| | indol-2-yl}prop-2-yn-1-yl)amino]-3-methoxybenzoate |
| 614. | 2-{3-[(4-methanesulfonyl-2-methoxyphenyl)amino]prop-1-yn-1-yl}-N-[1-(2- |
| | methanesulfonylethyl)-piperidin-4-yl]-1-(2,2,2-trifluoroethyl)-1H-indol-4-amine |
| 615. | 2-{3-[(4-methanesulfonyl-2-methoxyphenyl)amino]prop-1-yn-1-yl}-N-[1-(2- |
| | methoxyethyl)piperidin-4-yl]-1-(2,2,2-trifluoroethyl)-1H-indol-4-amine |
| 616. | (1S,4S)-4-N-(2-{3-[(4-methanesulfonyl-2-methoxyphenyl)amino]prop-1-yn-1-yl}- |
| | 1-(2,2,2-trifluoroethyl)-1H-indol-4-yl)-1-N,1-N-dimethylcyclohexane-1,4-diamine |
| 617. | (1R,4R)-4-N-(2-{3-[(4-methanesulfonyl-2-methoxyphenyl)amino]prop-1-yn-1- |
| | yl}-1-(2,2,2-trifluoroethyl)-1H-indol-4-yl)-1-N,1-N-dimethylcyclohexane-1,4- |
| | diamine |
| 618. | 2-{3-[(4-methanesulfonyl-2-methoxyphenyl)amino]prop-1-yn-1-yl}-N-[1-(oxan- |
| | 4-yl)piperidin-4-yl]-1-(2,2,2-trifluoroethyl)-1H-indol-4-amine |
| 619. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-2-0 |
| 620. | 2-{3-[(4-methanesulfonyl-2-methoxyphenyl)amino]prop-1-yn-1-yl}-N-[1-(1- |
| | methylpyrrolidin-3-yl)piperidin-4-yl]-1-(2,2,2-trifluoroethyl)-1H-indol-4-amine |
| 621. | 2-{3-[(4-methanesulfonyl-2-methoxyphenyl)amino]prop-1-yn-1-yl}-N-(2- |
| | methylpiperidin-4-yl)-1-(2,2,2-trifluoroethyl)-1H-indol-4-amine |
| 622. | 2-{3-[(4-methanesulfonyl-2-methoxyphenyl)amino]prop-1-yn-1-yl}-N-(piperidin- |
| | 4-yl)-1-(2,2,2-trifluoroethyl)-1H-indol-4-amine |
| 623. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-1-(4-methylpiperazin- |
| | 1-yl)ethan-1-one |
| 624. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}acetamide |
| 625. | 2-{3-[(4-methanesulfonyl-2-methoxyphenyl)amino]prop-1-yn-1-yl}-N-[1-(1- |
| | methylpiperidin-4-yl)piperidin-4-yl]-1-(2,2,2-trifluoroethyl)-1H-indol-4-amine |
| 626. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl}piperidin-4-ol |
| 627. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-1-(morpholin-4- |
| | yl)ethan-1-one |
| 628. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-N,N- |
| | dimethylacetamide |
| 629. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}acetonitrile |
| 630. | methyl 2-{4-[(2-{3-[(4-methane-sulfonyl-2-methoxyphenyl)amino]prop-1-yn-1- |
| | yl}-1-(2,2,2-trifluoroethyl)-1H-indol-4-yl)amino]piperidin-1-yl}acetate |
| 631. | 1-(4-hydroxypiperidin-1-yl)-2-{4-[(2-{3-[(4-methanesulfonyl-2-methoxy- |
| | phenyl)amino]prop-1-yn-1-yl}-1-(2,2,2-trifluoroethyl)-1H-indol-4- |
| | yl)amino]piperidin-1-yl}ethan-1-one |
| 632. | 2-(2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethoxy)ethan-1-ol |
| 633. | -[(1R,4R)-4-[(2-{3-[(4-methane-sulfonyl-2-methoxyphenyl)amino]prop-1-yn-1- |
| | yl}-1-(2,2,2-trifluoroethyl)-1H-indol-4-yl)amino]cyclohexyl]piperidin-4-ol |
| 634. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}acetic acid |
| 635. | (1R,4R)-4-[(2-{3-[(4-methanesulfonyl-2-methoxyphenyl)amino]prop-1-yn-1-yl}- |
| | 1-(2,2,2-trifluoroethyl)-1H-indol-4-yl)amino]cyclohexan-1-ol |
| 636. | (1S,4S)-4-[(2-{3-[(4-methanesulfonyl-2-methoxyphenyl)amino]prop-1-yn-1-yl}- |
| | 1-(2,2,2-trifluoroethyl)-1H-indol-4-yl)amino]cyclohexan-1-ol |
| 637. | 2-{3-[(4-methanesulfonyl-2-methoxyphenyl)amino]prop-1-yn-1-yl}-1-(2,2,2- |
| | trifluoroethyl)-1H-indol-4-amine |
| 638. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl}-3-methylpyrrolidin-3-ol |
| 639. | (3R,4R)-1-{4-[(2-{3-[(4-methane-sulfonyl-2-methoxyphenyl)amino]prop-1-yn-1- |
| | yl}-1-(2,2,2-trifluoroethyl)-1H-indol-4-yl)amino]cyclohexyl}-pyrrolidine-3,4-diol |
| 640. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carboximidamide |
| 641. | 1-[(1S,4S)-4-[(2-{3-[(4-methane-sulfonyl-2-methoxyphenyl)amino]prop-1-yn-1- |
| | yl}-1-(2,2,2-trifluoroethyl)-1H-indol-4-yl)amino]cyclohexyl]piperidin-4-ol |
| 642. | 4-[(2-{3-[(3-methoxypyridin-4-yl)amino]prop-1-yn-1-yl}-1-(2,2,2-trifluoroethyl)- |
| | 1H-indol-4-yl)amino]-1λ⁶-thiane-1,1-dione |
| 643. | 2-{3-[(4-methanesulfonyl-2-methoxyphenyl)amino]prop-1-yn-1-yl}-N-[(1S,4S)-4- |
| | (morpholin-4-yl)cyclohexyl]-1-(2,2,2-trifluoroethyl)-1H-indol-4-amine |
| 644. | 2-{3-[(4-methanesulfonyl-2-methoxyphenyl)amino]prop-1-yn-1-yl}-N-[(1R,4R)- |
| | 4-(morpholin-4-yl)cyclohexyl]-1-(2,2,2-trifluoroethyl)-1H-indol-4-amine |
| 645. | 2-(3-{[2-(2-fluoroethoxy)-4-methanesulfonylphenyl]amino}prop-1-yn-1-yl)-N-[1- |
| | (oxan-4-yl)piperidin-4-yl]-1-(2,2,2-trifluoroethyl)-1H-indol-4-amine |
| 646. | 4-{[2-(3-{[4-methanesulfonyl-2-(trifluoromethyl)phenyl]amino}prop-1-yn-1-yl)- |
| | 1-(2,2,2-trifluoroethyl)-1H-indol-4-yl]amino}-1λ⁶-thiane-1,1-dione |
| 647. | 2-(3-{[4-methanesulfonyl-2-(trifluoromethyl)phenyl]amino}prop-1-yn-1-yl)-N-(1- |
| | methylpiperidin-4-yl)-1-(2,2,2-trifluoroethyl)-1H-indol-4-amine |
| 648. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethan-1-one |
| 649. | 3-methoxy-4-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}benzene-1-sulfonamide |
| 650. | 3-methoxy-4-[(3-{4-[(piperidin-4-yl)amino]-1-(2,2,2-trifluoroethyl)-1H-indol-2- |
| | yl}prop-2-yn-1-yl)amino]-benzamide |
| 651. | 3-methoxy-4-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}benzamide |
| 652. | 3-(4-{[2-(3-{[2-(2-fluoroethoxy)-4-methanesulfonylphenyl]amino}prop-1-yn-1- |
| | yl)-1-(2,2,2-trifluoroethyl)-1H-indol-4-yl]amino}piperidin-1-yl)propane-1,2-diol |
| 653. | 3-(4-{[2-(3-{[4-methanesulfonyl-2-(2-methoxyethoxy)phenyl]amino}prop-1-yn-1- |
| | yl)-1-(2,2,2-trifluoroethyl)-1H-indol-4-yl]amino}piperidin-1-yl)propane-1,2-diol |
| 654. | 2-(4-{[2-(3-{[4-methanesulfonyl-2-(2-methoxyethoxy)phenyl]amino}prop-1-yn-1- |
| | yl)-1-(2,2,2-trifluoroethyl)-1H-indol-4-yl]amino}piperidin-1-yl)acetamide |
| 655. | 2-(4-{[2-(3-{[2-(2-fluoroethoxy)-4-methanesulfonylphenyl]amino}prop-1-yn-1- |
| | yl)-1-(2,2,2-trifluoroethyl)-1H-indol-4-yl]amino}piperidin-1-yl)-1-(4- |
| | methylpiperazin-1-yl)ethan-1-one |
| 656. | 2-(3-{[2-(2-fluoroethoxy)-4-methanesulfonylphenyl]amino}prop-1-yn-1-yl)-N- |
| | (oxan-4-yl)-1-(2,2,2-trifluoroethyl)-1H-indol-4-amine |
| 657. | 2-(3-{[2-(2-fluoroethoxy)-4-methanesulfonylphenyl]amino}prop-1-yn-1-yl)-N-[1- |
| | (1-methylpiperidin-4-yl)piperidin-4-yl]-1-(2,2,2-trifluoroethyl)-1H-indol-4-amine |
| 658. | 4-[(2-{3-[(4-methoxypyridin-3-yl)amino]prop-1-yn-1-yl}-1-(2,2,2-trifluoroethyl)- |
| | 1H-indol-4-yl)amino]-1λ⁶-thiane-1,1-dione |
| 659. | S-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}-2-hydroxyethane-1-sulfonamido |
| 660. | 2-hydroxy-S-{3-methoxy-4-[(3-{4-[(1-methylpiperidin-4-yl)amino]-1-(2,2,2- |
| | trifluoroethyl)-1H-indol-2-yl}prop-2-yn-1-yl)amino]phenyl}ethane-1-sulfonamido |
| 661. | 2-methyl-2-[5-({3-[4-(morpholin-4-yl)-1-(2,2,2-trifluoroethyl)-1H-indol-2- |
| | yl]prop-2-yn-1-yl}amino)pyridin-2-yl]propanenitrile |
| 662. | -{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}-N-[5-(2-{4-[(2-{3-[(4- |
| | methanesulfonylphenyl)amino]prop-1-yn-1-yl}-1-(2,2,2-trifluoroethyl)-1H-indol- |
| | 4-yl)amino]piperidin-1-yl}acetamido)pentyl]acetamide |
| 663. | 6-[(2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetyl)oxy]hexyl 2-{4-[(2-{3- |
| | [(4-methanesulfonylphenyl)amino]prop-1-yn-1-yl}-1-(2,2,2-trifluoroethyl)-1H- |
| | indol-4-yl)amino]piperidin-1-yl}acetate |
| 664. | 3-methoxy-4-({3-[4-({1-[2-(4-methylpiperazin-1-yl)-2-oxoethyl]piperidin-4- |
| | yl}amino)-1-(2,2,2-trifluoroethyl)-1H-indol-2-yl]prop-2-yn-1-yl}amino)benzene- |
| | 1-sulfonamide |
| 665. | 2-{5-methanesulfonyl-2-[(3-{4-[(1-methylpiperidin-4-yl)amino]-1-(2,2,2- |
| | trifluoroethyl)-1H-indol-2-yl}prop-2-yn-1-yl)amino]phenoxy}acetamide |
| 666. | 2-(3-{[4-methanesulfonyl-2-(2-methoxyethoxy)phenyl]amino}prop-1-yn-1-yl)-N- |
| | [1-(oxan-4-yl)piperidin-4-yl]-1-(2,2,2-trifluoroethyl)-1H-indol-4-amine |
| 667. | 2-(4-{[2-(3-{[4-methanesulfonyl-2-(2-methoxyethoxy)phenyl]amino}prop-1-yn-1- |
| | yl)-1-(2,2,2-trifluoroethyl)-1H-indol-4-yl]amino}piperidin-1-yl)ethan-1-ol |
| 668. | 3-methoxy-4-[(3-{4-[(oxan-4-yl)amino]-1-(2,2,2-trifluoroethyl)-1H-indol-2- |
| | yl}prop-2-yn-1-yl)amino]benzoic acid |
| 669. | 2-{2-[(3-{4-[(1, 1-dioxo-1λ⁶-thian-4-yl)amino]-1-(2,2,2-trifluoroethyl)-1H-indol-2- |
| | yl}prop-2-yn-1-yl)amino]-5-methanesulfonylphenoxy}acetamide |
| 670. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carboxamide |
| 671. | 2-{3-[(4-methoxypyridin-3-yl)amino]prop-1-yn-1-yl}-N-(1-methylpiperidin-4-yl)- |
| | 1-(2,2,2-trifluoroethyl)-1H-indol-4-amine |
| 672. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carbothioamide |
| 673. | 4-[(2-{3-[(6-methanesulfonyl-4-methoxypyridin-3-yl)amino]prop-1-yn-1-yl}-1- |
| | (2,2,2-trifluoroethyl)-1H-indol-4-yl)amino]-1λ⁶-thiane-1,1-dione |
| 674. | 3-methoxy-4-{[3-(4-{[1-(2-methoxyethyl)piperidin-4-yl]amino}-1-(2,2,2- |
| | trifluoroethyl)-1H-indol-2-yl)prop-2-yn-1-yl]amino}benzene-1-sulfonamide |
| 675. | 4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}-1λ⁶-thiane-1,1-dione |
| 676. | 2-(3-{[4-(ethanesulfonyl)-2-methoxyphenyl]amino}prop-1-yn-1-yl)-N-[1-(oxan-4- |
| | yl)piperidin-4-yl]-1-(2,2,2-trifluoroethyl)-1H-indol-4-amine |
| 677. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxybenzoic acid |
| 678. | methyl 4-{[3-(4-{[1-(2,3-dihydroxypropyl)piperidin-4-yl]amino}-1-(2,2,2- |
| | trifluoroethyl)-1H-indol-2-yl)prop-2-yn-1-yl]amino}-3-methoxybenzoate |
| 679. | methyl 3-methoxy-4-[(3-{4-[(oxan-4-yl)amino]-1-(2,2,2-trifluoroethyl)-1H-indol- |
| | 2-yl}prop-2-yn-1-yl)amino]benzoate |
| 680. | 3-methoxy-4-({3-[4-({1-[2-(4-methylpiperazin-1-yl)-2-oxoethyl]piperidin-4- |
| | yl}amino)-1-(2,2,2-trifluoroethyl)-1H-indol-2-yl]prop-2-yn-1- |
| | yl}amino)benzamide |
| 681. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]-N-methylpiperidine-1-carboximidamide |
| 682. | 2-{3-[(6-methanesulfonyl-4-methoxypyridin-3-yl)amino]prop-1-yn-1-yl}-N-(1- |
| | methylpiperidin-4-yl)-1-(2,2,2-trifluoroethyl)-1H-indol-4-amine |
| 683. | 2-{3-[(4-methanesulfonyl-2-methoxyphenyl)amino]prop-1-yn-1-yl}-N-[1- |
| | (pyridin-4-yl)piperidin-4-yl]-1-(2,2,2-trifluoroethyl)-1H-indol-4-amine |
| 684. | 3-(4-{[2-(3-{[4-(ethanesulfonyl)-2-methoxyphenyl]amino}prop-1-yn-1-yl)-1- |
| | (2,2,2-trifluoroethyl)-1H-indol-4-yl]amino}piperidin-1-yl)propane-1,2-diol |
| 685. | 2-(3-{[4-(ethanesulfonyl)-2-methoxyphenyl]amino}prop-1-yn-1-yl)-N-(1- |
| | methylpiperidin-4-yl)-1-(2,2,2-trifluoroethyl)-1H-indol-4-amine |
| 686. | 2-hydroxy-S-{3-methoxy-4-[(3-{4-[(oxan-4-yl)amino]-1-(2,2,2-trifluoroethyl)- |
| | 1H-indol-2-yl}prop-2-yn-1-yl)amino]phenyl}ethane-1-sulfonamido |
| 687. | 2-(3-{[4-methanesulfonyl-2-(2,2,2-trifluoroethoxy)phenyl]amino}prop-1-yn-1-yl)- |
| | N-(1-methylpiperidin-4-yl)-1-(2,2,2-trifluoroethyl)-1H-indol-4-amine |
| 688. | 4-{[2-(3-{[4-methanesulfonyl-2-(2,2,2-trifluoroethoxy)phenyl]amino}prop-1-yn- |
| | 1-yl)-1-(2,2,2-trifluoroethyl)-1H-indol-4-yl]amino}-1λ⁶-thiane-1,1-dione |
| 689. | 2-hydroxy-S-(3-methoxy-4-{[3-(4-{[1-(oxan-4-yl)piperidin-4-yl]amino}-1-(2,2,2- |
| | trifluoroethyl)-1H-indol-2-yl)prop-2-yn-1-yl]amino}phenyl)ethane-1-sulfonamido |
| 690. | S-(4-{[3-(4-{[1-(2,3-dihydroxypropyl)piperidin-4-yl]amino}-1-(2,2,2- |
| | trifluoroethyl)-1H-indol-2-yl)prop-2-yn-1-yl]amino}-3-methoxyphenyl)-2- |
| | hydroxyethane-1-sulfonamido |
| 691. | 2-{3-methoxy-4-[(3-{4-[(1-methylpiperidin-4-yl)amino]-1-(2,2,2-trifluoroethyl)- |
| | 1H-indol-2-yl}prop-2-yn-1-yl)amino]phenyl}-2-methylpropanenitrile |
| 692. | 3-methoxy-4-{[3-(4-{[(1S,4S)-4-[(3R,4R)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 693. | 3-methoxy-4-{[3-(4-{[(1R,4R)-4-[(3R,4R)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 694. | 3-methoxy-4-{[3-(4-{[(1S,4S)-4-(4-hydroxypiperidin-1-yl)cyclohexyl]amino}-1- |
| | (2,2,2-trifluoroethyl)-1H-indol-2-yl)prop-2-yn-1-yl]amino}benzene-1-sulfonamide |
| 695. | 3-methoxy-4-{[3-(4-{[(1R,4R)-4-(4-hydroxypiperidin-1-yl)cyclohexyl]amino}-1- |
| | (2,2,2-trifluoroethyl)-1H-indol-2-yl)prop-2-yn-1-yl]amino}benzene-1-sulfonamide |
| 696. | 2-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}-2-methylpropanenitrile |
| 697. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 698. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 699. | (3S,4S)-1-[(1S,4S)-4-[(2-{3-[(4-methanesulfonyl-2-methoxyphenyl)amino]prop- |
| | 1-yn-1-yl}-1-(2,2,2-trifluoroethyl)-1H-indol-4-yl)amino]cyclohexyl]pyrrolidine- |
| | 3,4-diol |
| 700. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 701. | 3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 702. | 2-{3-[(4-methanesulfonyl-2-methoxyphenyl)amino]prop-1-yn-1-yl}-N,N- |
| | dimethyl-1-(2,2,2-trifluoroethyl)-1H-indol-4-amine |
| 703. | 3-methoxy-4-[(3-{4-[(2-methoxyethyl)amino]-1-(2,2,2-trifluoroethyl)-1H-indol-2- |
| | yl}prop-2-yn-1-yl)amino]benzene-1-sulfonamide |
| 704. | 2-[(2-{3-[(2-methoxy-4-sulfamoylphenyl)amino]prop-1-yn-1-yl}-1-(2,2,2- |
| | trifluoroethyl)-1H-indol-4-yl)amino]acetamide |
| 705. | 4-[(3-{4-[(1,5-dihydroxypentan-3-yl)amino]-1-(2,2,2-trifluoroethyl)-1H-indol-2- |
| | yl}prop-2-yn-1-yl)amino]-3-methoxybenzene-1-sulfonamide |
| 706. | N-(2-{3-[(4-methanesulfonyl-2-methoxyphenyl)amino]prop-1-yn-1-yl}-1-(2,2,2- |
| | trifluoroethyl)-1H-indol-4-yl)acetamide |
| 707. | 3-(2-{3-[(4-methanesulfonyl-2-methoxyphenyl)amino]prop-1-yn-1-yl}-1-(2,2,2- |
| | trifluoroethyl)-1H-indol-4-yl)-1-(1-methylpiperidin-4-yl)urea |
| 708. | 3-methoxy-4-{[3-(4-{[(1-methylpiperidin-4-yl)carbamoyl]amino}-1-(2,2,2- |
| | trifluoroethyl)-1H-indol-2-yl)prop-2-yn-1-yl]amino}benzamide |
| 709. | N-(2-{3-[(4-methanesulfonyl-2-methoxyphenyl)amino]prop-1-yn-1-yl}-1-(2,2,2- |
| | trifluoroethyl)-1H-indol-4-yl)-4-methylpiperazine-1-carboxamide |
| 710. | N-(2-{3-[(4-carbamoyl-2-methoxyphenyl)amino]prop-1-yn-1-yl}-1-(2,2,2- |
| | trifluoroethyl)-1H-indol-4-yl)-4-methylpiperazine-1-carboxamide |
| 711. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[(1S,4S)-4-(dimethylamino)cyclohexyl]urea |
| 712. | 1-(2-(3-((2-methoxy-4-(methylsulfonyl)phenyl)amino)prop-1-yn-1-yl)-1-(2,2,2- |
| | trifluoroethyl)-1H-indol-4-yl)-3-(pyridin-4-yl)urea |
| 713. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[(1R,4R)-4-(dimethylamino)cyclohexyl]urea |
| 714. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[1-(2-methoxyethyl)piperidin-4-yl]urea |
| 715. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[1-(oxan-4-yl)piperidin-4-yl]urea |
| 716. | 1-[1-(2,3-dihydroxypropyl)piperidin-4-yl]-3-(2-{3-[(4-methanesulfonyl-2- |
| | methoxyphenyl)amino]prop-1-yn-1-yl}-1-(2,2,2-trifluoroethyl)-1H-indol-4- |
| | yl)urea |
| 717. | 2-(4-{[(2-{3-[(4-methanesulfonyl-2-methoxyphenyl)amino]prop-1-yn-1-yl}-1- |
| | (2,2,2-trifluoroethyl)-1H-indol-4-yl)carbamoyl]amino}piperidin-1-yl)acetamide |
| 718. | 3-methoxy-4-({3-[4-({2-oxaspiro[3.3]heptan-6-yl}amino)-1-(2,2,2-trifluoroethyl)- |
| | 1H-indol-2-yl]prop-2-yn-1-yl}amino)benzene-1-sulfonamide |
| 719. | [1-(chloromethyl)-3-[(2-{3-[(4-methanesulfonyl-2-methoxyphenyl)amino]prop-1- |
| | yn-1-yl}-1-(2,2,2-trifluoroethyl)-1H-indol-4-yl)amino]cyclobutyl]methanol |
| 720. | 2-{3-[(4-methanesulfonyl-2-methoxyphenyl)amino]prop-1-yn-1-yl}-N-{2- |
| | oxaspiro[3.3]heptan-6-yl}-1-(2,2,2-trifluoroethyl)-1H-indol-4-amine |
| 721. | 4-({3-[4-({2-azaspiro[3.3]heptan-6-yl}amino)-1-(2,2,2-trifluoroethyl)-1H-indol-2- |
| | yl]prop-2-yn-1-yl}amino)-3-methoxybenzene-1-sulfonamide |
| 722. | N-{2-azaspiro[3.3]heptan-6-yl}-2-{3-[(4-methanesulfonyl-2- |
| | methoxyphenyl)amino]prop-1-yn-1-yl}-1-(2,2,2-trifluoroethyl)-1H-indol-4-amine |
| 723. | 3-methoxy-N-methyl-4-{[3-(4-{[(1R,4R)-4-(methylamino)cyclohexyl]amino}-1- |
| | (2,2,2-trifluoroethyl)-1H-indol-2-yl)prop-2-yn-1-yl]amino}benzamide |
| 724. | 3-methoxy-N-methyl-4-{[3-(4-{[(1R,4R)-4-aminocyclohexyl]amino}-1-(2,2,2- |
| | trifluoroethyl)-1H-indol-2-yl)prop-2-yn-1-yl]amino}benzamide |
| 725. | 3-methoxy-N-methyl-4-{[3-(4-{[(1S,4S)-4-aminocyclohexyl]amino}-1-(2,2,2- |
| | trifluoroethyl)-1H-indol-2-yl)prop-2-yn-1-yl]amino}benzamide |
| 726. | rel-(1R,3R)-N¹-(2-{3-[(4-methanesulfonyl-2-methoxyphenyl)amino]prop-1-yn-1- |
| | yl}-1-(2,2,2-trifluoroethyl)-1H-indol-4-yl)cyclohexane-1,3-diamine |
| 727. | rac-(1R,3S)-N¹-(2-{3-[(4-methanesulfonyl-2-methoxyphenyl)amino]prop-1-yn-1- |
| | yl}-1-(2,2,2-trifluoroethyl)-1H-indol-4-yl)cyclohexane-1,3-diamine |
| 728. | (1R,2S)-N¹-(2-{3-[(4-methanesulfonyl-2-methoxyphenyl)amino]prop-1-yn-1-yl}- |
| | 1-(2,2,2-trifluoroethyl)-1H-indol-4-yl)cyclohexane-1,2-diamineQ |
| 729. | rac-(1R,2S)-N¹-(2-{3-[(4-methanesulfonyl-2-methoxyphenyl)amino]prop-1-yn-1- |
| | yl}-1-(2,2,2-trifluoroethyl)-1H-indol-4-yl)cyclohexane-1,2-diamine |
| 730. | rel-(1R,3S)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexan-1-ol |
| 731. | 3-methoxy-N-methyl-4-{[3-(4-{[(1R,4R)-4-hydroxy-4-methylcyclohexyl]amino}- |
| | 1-(2,2,2-trifluoroethyl)-1H-indol-2-yl)prop-2-yn-1-yl]amino}benzamide |
| 732. | 3-methoxy-N-methyl-4-{[3-(4-{[(1S,4S)-4-hydroxy-4-methylcyclohexyl]amino}- |
| | 1-(2,2,2-trifluoroethyl)-1H-indol-2-yl)prop-2-yn-1-yl]amino}benzamide |
| 733. | 4-[(3-{4-[(4-cyanocyclohexyl)amino]-1-(2,2,2-trifluoroethyl)-1H-indol-2-yl}prop- |
| | 2-yn-1-yl)amino]-3-methoxy-N-methylbenzamide |
| 734. | 3-methoxy-N-methyl-4-{[3-(4-{[(1R,4R)-4-cyanocyclohexyl]amino}-1-(2,2,2- |
| | trifluoroethyl)-1H-indol-2-yl)prop-2-yn-1-yl]amino}benzamide |
| 735. | 3-methoxy-N-methyl-4-{[3-(4-{[(1S,4S)-4-cyanocyclohexyl]amino}-1-(2,2,2- |
| | trifluoroethyl)-1H-indol-2-yl)prop-2-yn-1-yl]amino}benzamide |
| 736. | 3-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexane-1-carboxylic acid |
| 737. | 2-fluoro-N¹-(2-(3-((2-methoxy-4-(methylsulfonyl)phenyl)amino)prop-1-yn-1-yl)- |
| | 1-(2,2,2-trifluoroethyl)-1H-indol-4-yl)cyclohexane-1,4-diamine |
| 738. | (1R,2R,4S)-2-fluoro-N¹-(2-(3-((2-methoxy-4-(methylsulfonyl)phenyl)amino)prop- |
| | 1-yn-1-yl)-1-(2,2,2-trifluoroethyl)-1H-indol-4-yl)cyclohexane-1,4-diamine |
| 739. | 2-fluoro-N¹-(2-{3-[(4-methanesulfonyl-2-methoxyphenyl)amino]prop-1-yn-1-yl}- |
| | 1-(2,2,2-trifluoroethyl)-1H-indol-4-yl)-N⁴-methylcyclohexane-1,4-diamine |
| 740. | (1R,2R,4S)-2-fluoro-N¹-(2-(3-((2-methoxy-4-(methylsulfonyl)phenyl)amino)prop- |
| | 1-yn-1-yl)-1-(2,2,2-trifluoroethyl)-1H-indol-4-yl)-N⁴-methylcyclohexane-1,4- |
| | diamine |
| 741. | 2-fluoro-N¹-(2-(3-((2-methoxy-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 742. | (1R,2R,4S)-2-fluoro-N¹-(2-(3-((2-methoxy-4-(methylsulfonyl)phenyl)amino)prop- |
| | 1-yn-1-yl)-1-(2,2,2-trifluoroethyl)-1H-indol-4-yl)-N⁴,N⁴-dimethylcyclohexane- |
| | 1,4-diamine |
| 743. | (3S,4R)-3-fluoro-4-[(2-{3-[(4-methanesulfonyl-2-methoxyphenyl)amino]prop-1- |
| | yn-1-yl}-1-(2,2,2-trifluoroethyl)-1H-indol-4-yl)amino]cyclohexan-1-ol |
| 744. | 3-methoxy-4-{[3-(4-{[(1R,4R)-4-[bis(2-hydroxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 745. | 3-methoxy-4-{[3-(4-{[(1S,4S)-4-[bis(2-hydroxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 746. | 4-((3-(4-(((1R,4R)-4-(dimethylamino)cyclohexyl)amino)-1-(2,2,2-trifluoroethyl)- |
| | 1H-indol-2-yl)prop-2-yn-1-yl)amino)-3-methoxybenzenesulfonamide |
| 747. | 4-((3-(4-(((1S,4S)-4-(dimethylamino)cyclohexyl)amino)-1-(2,2,2-trifluoroethyl)- |
| | 1H-indol-2-yl)prop-2-yn-1-yl)amino)-3-methoxybenzenesulfonamide |
| 748. | 3-methoxy-N-(1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 749. | 3-methoxy-N-(oxan-4-yl)-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 750. | 3-methoxy-N-(1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 751. | 3-methoxy-N-(oxan-4-yl)-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 752. | N-(3-(4-(((1S,4S)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)propionamide |
| 753. | N-(3-(4-(((1R,4R)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)propionamide |
| 754. | 3-methoxy-N-(5-methyl-1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 755. | 3-methoxy-N-(5-methyl-1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 756. | (1S,4S)-N⁴-(2-{3-[(4-methanesulfonyl-2-methoxyphenyl)amino]prop-1-yn-1-yl}- |
| | 1-(2,2,2-trifluoroethyl)-1H-indol-4-yl)-N¹-(2-methoxyethyl)-N¹- |
| | methylcyclohexane-1,4-diamine |
| 757. | (1R,4R)-N⁴-(2-{3-[(4-methanesulfonyl-2-methoxyphenyl)amino]prop-1-yn-1-yl}- |
| | 1-(2,2,2-trifluoroethyl)-1H-indol-4-yl)-N¹-(2-methoxyethyl)-N¹- |
| | methylcyclohexane-1,4-diamine |
| 758. | 3-methoxy-4-{[3-(4-{[(1S,4S)-4-[(3S,4S)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 759. | 4-((3-(4-(((1R,4R)-4-((3R,4R)-3,4-dihydroxypyrrolidin-1-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 760. | 4-((3-(4-(((1R,4S)-4-((3S,4S)-3,4-dihydroxypyrrolidin-1-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 761. | 3-methoxy-4-{[3-(4-{[(1R,4R)-4-[bis(2-methoxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 762. | 3-methoxy-4-{[3-(4-{[(1S,4S)-4-[bis(2-methoxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 763. | 4-({3-[4-({1,4-dioxaspiro[4.5]decan-8-yl}amino)-1-(2,2,2-trifluoroethyl)-1H- |
| | indol-2-yl]prop-2-yn-1-yl}amino)-3-methoxybenzene-1-sulfonamide |
| 764. | N-{1,4-dioxaspiro[4.5]decan-8-yl}-2-{3-[(4-methanesulfonyl-2- |
| | methoxyphenyl)amino]prop-1-yn-1-yl}-1-(2,2,2-trifluoroethyl)-1H-indol-4-amine |
| 765. | 3-methoxy-4-[(3-{4-[(4-oxocyclohexyl)amino]-1-(2,2,2-trifluoroethyl)-1H-indol- |
| | 2-yl}prop-2-yn-1-yl)amino]benzene-1-sulfonamide |
| 766. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-one |
| 767. | (1R,4R)-N⁴-[2-(3-{[2-methoxy-4-(morpholine-4-sulfonyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 768. | (1S,4S)-N⁴-[2-(3-{[2-methoxy-4-(morpholine-4-sulfonyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 769. | N,N-bis(2-hydroxyethyl)-3-methoxy-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 770. | N,N-bis(2-hydroxyethyl)-3-methoxy-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 771. | 3-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzene-1- |
| | sulfonamide |
| 772. | 3-methoxy-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 773. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 774. | 3-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 775. | 3-methoxy-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 776. | (1R,4R)-N⁴-{2-[3-({2-methoxy-4-[(4-methylpiperazin-1- |
| | yl)sulfonyl]phenyl}amino)prop-1-yn-1-yl]-1-(2,2,2-trifluoroethyl)-1H-indol-4-yl}- |
| | N¹,N¹-dimethylcyclohexane-1,4-diamine |
| 777. | (1R,4R)-N⁴-(2-{3-[(4-methanesulfonyl-2-methoxyphenyl)amino]prop-1-yn-1-yl}- |
| | 1-[(oxiran-2-yl)methyl]-1H-indol-4-yl)-N¹,N¹-dimethylcyclohexane-1,4-diamine |
| 778. | 2-[(2-{3-[(4-methanesulfonyl-2-methoxyphenyl)amino]prop-1-yn-1-yl}-4- |
| | {[(1R,4R)-4-(dimethylamino)cyclohexyl]amino}-1H-indol-1-yl)methyl]prop-2- |
| | enenitrile |
| 779. | 5-methanesulfonyl-2-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}phenol |
| 780. | 5-methanesulfonyl-2-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}phenol |
| 781. | 2-(5-methanesulfonyl-2-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}phenoxy)acetonitrile |
| 782. | 2-(5-methanesulfonyl-2-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}phenoxy)acetonitrile |
| 783. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 784. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 785. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 786. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 787. | 2-(5-methanesulfonyl-2-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}phenoxy)ethan-1-ol |
| 788. | 2-(5-methanesulfonyl-2-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}phenoxy)ethan-1-ol |
| 789. | (1R,4R)-N⁴-[2-(3-{[4-methanesulfonyl-2-(2-methoxyethoxy)phenyl]amino}prop- |
| | 1-yn-1-yl)-1-(2,2,2-trifluoroethyl)-1H-indol-4-yl]-N¹,N¹-dimethylcyclohexane- |
| | 1,4-diamine |
| 790. | N-(3-(4-(((1R,4R)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)isobutyramide |
| 791. | N-(3-(4-(((1S,4S)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)isobutyramide |
| 792. | (1R,4R)-N⁴-[2-(3-{[4-methanesulfonyl-2-(2,2,2- |
| | trifluoroethoxy)phenyl]amino}prop-1-yn-1-yl)-1-(2,2,2-trifluoroethyl)-1H-indol- |
| | 4-yl]-N¹,N¹-dimethylcyclohexane-1,4-diamine |
| 793. | (1S,4S)-N⁴-[2-(3-{[4-methanesulfonyl-2-(2,2,2- |
| | trifluoroethoxy)phenyl]amino}prop-1-yn-1-yl)-1-(2,2,2-trifluoroethyl)-1H-indol- |
| | 4-yl]-N¹,N¹-dimethylcyclohexane-1,4-diamine |
| 794. | (1S,4S)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 795. | (1R,4R)-N⁴-(2-{3-[(2-ethoxy-4-methanesulfonylphenyl)amino]prop-1-yn-1-yl}-1- |
| | (2,2,2-trifluoroethyl)-1H-indol-4-yl)-N¹,N¹-dimethylcyclohexane-1,4-diamine |
| 796. | (1S,4S)-N⁴-(2-{3-[(2-ethoxy-4-methanesulfonylphenyl)amino]prop-1-yn-1-yl}-1- |
| | (2,2,2-trifluoroethyl)-1H-indol-4-yl)-N¹,N¹-dimethylcyclohexane-1,4-diamine |
| 797. | 3-hydroxy-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 798. | 3-hydroxy-N-methyl-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzamide |
| 799. | 3-(2-methoxyethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzamide |
| 800. | (1R,4R)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 801. | N-ethyl-3-methoxy-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzamide |
| 802. | N-ethyl-3-methoxy-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 803. | 3-(2-methoxyethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 804. | 3-(2-fluoroethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 805. | 3-(2-fluoroethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzamide |
| 806. | 3-(fluoromethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 807. | 3-(fluoromethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzamide |
| 808. | (1S,4S)-N⁴-(2-{3-[4-methanesulfonyl-2-(methylamino)phenoxy]prop-1-yn-1-yl}- |
| | 1-(2,2,2-trifluoroethyl)-1H-indol-4-yl)-N¹,N¹-dimethylcyclohexane-1,4-diamine |
| 809. | (1R,4R)-N⁴-(2-{3-[4-methanesulfonyl-2-(methylamino)phenoxy]prop-1-yn-1-yl}- |
| | 1-(2,2,2-trifluoroethyl)-1H-indol-4-yl)-N¹,N¹-dimethylcyclohexane-1,4-diamine |
| 810. | 3-methoxy-N-methyl-4-{[3-(4-{[(1S,4S)-4-(methylamino)cyclohexyl]amino}-1- |
| | (2,2,2-trifluoroethyl)-1H-indol-2-yl)prop-2-yn-1-yl]amino}benzamide |
| 811. | (1S,4S)-N⁴-(2-{3-[2-(dimethylamino)-4-methanesulfonylphenoxy]prop-1-yn-1- |
| | yl}-1-(2,2,2-trifluoroethyl)-1H-indol-4-yl)-N¹,N¹-dimethylcyclohexane-1,4- |
| | diamine |
| 812. | (1R,4R)-N⁴-(2-{3-[2-(dimethylamino)-4-methanesulfonylphenoxy]prop-1-yn-1- |
| | yl}-1-(2,2,2-trifluoroethyl)-1H-indol-4-yl)-N¹,N¹-dimethylcyclohexane-1,4- |
| | diamine |
| 813. | 3-(cyanomethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 814. | 3-(2-cyanoethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 815. | N-(5-methanesulfonyl-2-{[3-(4-{[(1R,4R)-4-(dimethylamino)cyclohexyl]amino}- |
| | 1-(2,2,2-trifluoroethyl)-1H-indol-2-yl)prop-2-yn-1-yl]oxy}phenyl)acetamide |
| 816. | N-(2-((3-(4-(((1S,4S)-4-(dimethylamino)cyclohexyl)amino)-1-(2,2,2- |
| | trifluoroethyl)-1H-indol-2-yl)prop-2-yn-1-yl)oxy)-5- |
| | (methylsulfonyl)phenyl)acetamide |
| 817. | (1R,4R)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 818. | (1S,4S)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 819. | (1R,4R)-N⁴-[2-(3-{[4-(ethanesulfonyl)-2-methoxyphenyl]amino}prop-1-yn-1-yl)- |
| | 1-(2,2,2-trifluoroethyl)-1H-indol-4-yl]-N¹,N¹-dimethylcyclohexane-1,4-diamine |
| 820. | (1S,4S)-N⁴-[2-(3-{[4-(ethanesulfonyl)-2-methoxyphenyl]amino}prop-1-yn-1-yl)- |
| | 1-(2,2,2-trifluoroethyl)-1H-indol-4-yl]-N¹,N¹-dimethylcyclohexane-1,4-diamine |
| 821. | 2-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenyl)-2- |
| | methylpropanenitrile |
| 822. | 2-(4-((3-(4-(((1S,4S)-4-(dimethylamino)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 823. | 3-(cyanomethoxy)-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 824. | 3-(cyanomethoxy)-4-((3-(4-(((1S,4S)-4-(dimethylamino)cyclohexyl)amino)-1- |
| | (2,2,2-trifluoroethyl)-1H-indol-2-yl)prop-2-yn-1-yl)amino)benzenesulfonamide |
| 825. | 3-(3-(4-(((1R,4R)-4-(dimethylamino)cyclohexyl)amino)-1-(2,2,2-trifluoroethyl)- |
| | 1H-indol-2-yl)prop-2-yn-1-yl)-6-(methylsulfonyl)benzo[d]oxazol-2(3H)-one |
| 826. | 3-(3-(4-(((1S,4S)-4-(dimethylamino)cyclohexyl)amino)-1-(2,2,2-trifluoroethyl)- |
| | 1H-indol-2-yl)prop-2-yn-1-yl)-6-(methylsulfonyl)benzo[d]oxazol-2(3H)-one |
| 827. | 3-methoxy-N,N-dimethyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 828. | (1R,4R)-N¹-(2-(3-(((3S,4R)-3-methoxytetrahydro-2H-pyran-4-yl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 829. | (1S,4S)-N¹-(2-(3-(((3S,4R)-3-methoxytetrahydro-2H-pyran-4-yl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 830. | (1R,4R)-N⁴-(2-{3-[(4-chloro-2-methoxyphenyl)amino]prop-1-yn-1-yl}-1-(2,2,2- |
| | trifluoroethyl)-1H-indol-4-yl)-N¹,N¹-dimethylcyclohexane-1,4-diamine |
| 831. | (1S,4S)-N¹-(2-(3-((4-chloro-2-methoxyphenyl)amino)prop-1-yn-1-yl)-1-(2,2,2- |
| | trifluoroethyl)-1H-indol-4-yl)-N⁴,N⁴-dimethylcyclohexane-1,4-diamine |
| 832. | 4-((3-(4-(((1R,4R)-4-(dimethylamino)cyclohexyl)amino)-1-(2,2,2-trifluoroethyl)- |
| | 1H-indol-2-yl)prop-2-yn-1-yl)amino)-2-fluoro-5-methoxybenzamide |
| 833. | 4-((3-(4-(((1S,4S)-4-(dimethylamino)cyclohexyl)amino)-1-(2,2,2-trifluoroethyl)- |
| | 1H-indol-2-yl)prop-2-yn-1-yl)amino)-2-fluoro-5-methoxybenzamide |
| 834. | (1R,4R)-N⁴-[2-(3-{[2-methoxy-4-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 835. | (1S,4S)-N¹-(2-(3-((2-methoxy-4-(trifluoromethyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 836. | 2-fluoro-5-methoxy-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 837. | 4-((3-(4-(((1S,4S)-4-(dimethylamino)cyclohexyl)amino)-1-(2,2,2-trifluoroethyl)- |
| | 1H-indol-2-yl)prop-2-yn-1-yl)amino)-2-fluoro-5-methoxy-N-methylbenzamide |
| 838. | (1S,4S)-N⁴-[2-(3-{[4-methanesulfonyl-2-(trifluoromethyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 839. | (1R,4R)-N⁴-[2-(3-{[4-methanesulfonyl-2-(trifluoromethyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 840. | (1R,4R)-N⁴-(2-{3-[(4-methanesulfonyl-2-methylphenyl)amino]prop-1-yn-1-yl}-1- |
| | (2,2,2-trifluoroethyl)-1H-indol-4-yl)-N¹,N¹-dimethylcyclohexane-1,4-diamine |
| 841. | (1S,4S)-N¹,N¹-dimethyl-N⁴-(2-(3-((2-methyl-4- |
| | (methylsulfonyl)phenyl)amino)prop-1-yn-1-yl)-1-(2,2,2-trifluoroethyl)-1H-indol- |
| | 4-yl)cyclohexane-1,4-diamine |
| 842. | (1R,4R)-N⁴-[2-(3-{[4-methanesulfonyl-2-(trifluoromethoxy)phenyl]amino}prop- |
| | 1-yn-1-yl)-1-(2,2,2-trifluoroethyl)-1H-indol-4-yl]-N¹,N¹-dimethylcyclohexane- |
| | 1,4-diamine |
| 843. | (1S,4S)-N¹,N¹-dimethyl-N⁴-(2-(3-((4-(methylsulfonyl)-2- |
| | (trifluoromethoxy)phenyl)amino)prop-1-yn-1-yl)-1-(2,2,2-trifluoroethyl)-1H- |
| | indol-4-yl)cyclohexane-1,4-diamine |
| 844. | 3-methoxy-N-methyl-4-{[3-(4-{[(1R,4R)-4-(diethylamino)cyclohexyl]amino}-1- |
| | (2,2,2-trifluoroethyl)-1H-indol-2-yl)prop-2-yn-1-yl]amino}benzamide |
| 845. | 4-((3-(4-(((1S,4S)-4-(diethylamino)cyclohexyl)amino)-1-(2,2,2-trifluoroethyl)- |
| | 1H-indol-2-yl)prop-2-yn-1-yl)amino)-3-methoxy-N-methylbenzamide |
| 846. | 3-methoxy-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 847. | 4-((3-(4-(((1S,4S)-4-(diethylamino)cyclohexyl)amino)-1-(2,2,2-trifluoroethyl)- |
| | 1H-indol-2-yl)prop-2-yn-1-yl)amino)-3-methoxybenzenesulfonamide |
| 848. | 3-methoxy-N-methyl-4-{[3-(4-{[(1R,4R)-4-(diethylamino)cyclohexyl]amino}-1- |
| | (2,2,2-trifluoroethyl)-1H-indol-2-yl)prop-2-yn-1-yl]amino}benzene-1-sulfonamide |
| 849. | 2-(5-methanesulfonyl-2-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}phenoxy)acetonitrile |
| 850. | 2-(5-methanesulfonyl-2-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}phenoxy)acetonitrile |
| 851. | 3-methoxy-N-methyl-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 852. | 3-methoxy-N-methyl-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 853. | N-ethyl-3-methoxy-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 854. | N-ethyl-3-methoxy-4-((3-(4-(((1S,4S)-4-((2- |
| | methoxyethyl)(methyl)amino)cyclohexyl)amino)-1-(2,2,2-trifluoroethyl)-1H- |
| | indol-2-yl)prop-2-yn-1-yl)amino)benzamide |
| 855. | (1R,4R)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴-(2-methoxyethyl)-N⁴- |
| | methylcyclohexane-1,4-diamine |
| 856. | (1S,4S)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴-(2-methoxyethyl)-N⁴- |
| | methylcyclohexane-1,4-diamine |
| 857. | 3-(fluoromethoxy)-N-methyl-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 858. | 3-(fluoromethoxy)-N-methyl-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 859. | 3-(fluoromethoxy)-4-((3-(4-(((1S,4S)-4-((2- |
| | methoxyethyl)(methyl)amino)cyclohexyl)amino)-1-(2,2,2-trifluoroethyl)-1H- |
| | indol-2-yl)prop-2-yn-1-yl)amino)-N-methylbenzamide |
| 860. | 3-methoxy-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 861. | 3-methoxy-4-((3-(4-(((1S,4S)-4-((2- |
| | methoxyethyl)(methyl)amino)cyclohexyl)amino)-1-(2,2,2-trifluoroethyl)-1H- |
| | indol-2-yl)prop-2-yn-1-yl)amino)benzenesulfonamide |
| 862. | (1R,4R)-N¹,N¹-diethyl-N⁴-(2-{3-[(4-methanesulfonyl-2- |
| | methoxyphenyl)amino]prop-1-yn-1-yl}-1-(2,2,2-trifluoroethyl)-1H-indol-4- |
| | yl)cyclohexane-1,4-diamine |
| 863. | (1S,4S)-N¹,N¹-diethyl-N⁴-(2-(3-((2-methoxy-4- |
| | (methylsulfonyl)phenyl)amino)prop-1-yn-1-yl)-1-(2,2,2-trifluoroethyl)-1H-indol- |
| | 4-yl)cyclohexane-1,4-diamine |
| 864. | 3-(cyanomethoxy)-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 865. | 3-(cyanomethoxy)-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 866. | 3-(fluoromethoxy)-4-{[3-(4-{[(1R,4R)-4-[bis(2- |
| | methoxyethyl)amino]cyclohexyl]amino}-1-(2,2,2-trifluoroethyl)-1H-indol-2- |
| | yl)prop-2-yn-1-yl]amino}benzene-1-sulfonamide |
| 867. | 3-(fluoromethoxy)-4-{[3-(4-{[(1S,4S)-4-[bis(2- |
| | methoxyethyl)amino]cyclohexyl]amino}-1-(2,2,2-trifluoroethyl)-1H-indol-2- |
| | yl)prop-2-yn-1-yl]amino}benzene-1-sulfonamide |
| 868. | 3-(fluoromethoxy)-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 869. | 3-(fluoromethoxy)-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 870. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 871. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 872. | 3-methoxy-N-methyl-4-{[3-(4-{[(1R,4R)-4-(morpholin-4-yl)cyclohexyl]amino}- |
| | 1-(2,2,2-trifluoroethyl)-1H-indol-2-yl)prop-2-yn-1-yl]amino}benzamide |
| 873. | 3-methoxy-N-methyl-4-{[3-(4-{[(1S,4S)-4-(morpholin-4-yl)cyclohexyl]amino}-1- |
| | (2,2,2-trifluoroethyl)-1H-indol-2-yl)prop-2-yn-1-yl]amino}benzamide |
| 874. | 2-(2-{3-[(4-methanesulfonyl-2-methoxyphenyl)amino]prop-1-yn-1-yl}-4- |
| | {[(1R,4R)-4-(morpholin-4-yl)cyclohexyl]amino}-1H-indol-1-yl)acetonitrile |
| 875. | 4-({3-[1-(2-fluoroethyl)-4-{[(1R,4R)-4-(morpholin-4-yl)cyclohexyl]amino}-1H- |
| | indol-2-yl]prop-2-yn-1-yl}amino)-3-methoxy-N-methylbenzamide |
| 876. | 1-(2-fluoroethyl)-2-{3-[(4-methanesulfonyl-2-methoxyphenyl)amino]prop-1-yn-1- |
| | yl}-N-[(1R,4R)-4-(morpholin-4-yl)cyclohexyl]-1H-indol-4-amine |
| 877. | 4-({3-[1-(cyanomethyl)-4-{[(1R,4R)-4-(morpholin-4-yl)cyclohexyl]amino}-1H- |
| | indol-2-yl]prop-2-yn-1-yl}amino)-3-methoxy-N-methylbenzamide |
| 878. | 2-{3-[(4-methanesulfonyl-2-methoxyphenyl)amino]prop-1-yn-1-yl}-1-propyl-N- |
| | [(1R,4R)-4-(morpholin-4-yl)cyclohexyl]-1H-indol-4-amine |
| 879. | 2-{3-[(4-methanesulfonyl-2-methoxyphenyl)amino]prop-1-yn-1-yl}-1-(2- |
| | methylpropyl)-N-[(1R,4R)-4-(morpholin-4-yl)cyclohexyl]-1H-indol-4-amine |
| 880. | 1-(2,2-difluoroethyl)-2-{3-[(4-methanesulfonyl-2-methoxyphenyl)amino]prop-1- |
| | yn-1-yl}-N-[(1R,4R)-4-(morpholin-4-yl)cyclohexyl]-1H-indol-4-amine |
| 881. | 3-methoxy-4-((3-(4-(((1R,4R)-4-morpholinocyclohexyl)amino)-1-(2,2,2- |
| | trifluoroethyl)-1H-indol-2-yl)prop-2-yn-1-yl)amino)benzenesulfonamide |
| 882. | 3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzenesulfonamide |
| 883. | N-ethyl-3-methoxy-4-{[3-(4-{[(1R,4R)-4-(morpholin-4-yl)cyclohexyl]amino}-1- |
| | (2,2,2-trifluoroethyl)-1H-indol-2-yl)prop-2-yn-1-yl]amino}benzamide |
| 884. | N-ethyl-3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzamide |
| 885. | 2-(5-methanesulfonyl-2-{[3-(4-{[(1R,4R)-4-(morpholin-4-yl)cyclohexyl]amino}- |
| | 1-(2,2,2-trifluoroethyl)-1H-indol-2-yl)prop-2-yn-1-yl]amino}phenoxy)acetonitrile |
| 886. | 2-(5-methanesulfonyl-2-{[3-(4-{[(1S,4S)-4-(morpholin-4-yl)cyclohexyl]amino}- |
| | 1-(2,2,2-trifluoroethyl)-1H-indol-2-yl)prop-2-yn-1-yl]amino}phenoxy)acetonitrile |
| 887. | 3-methoxy-N-methyl-4-{[3-(4-{[(1R,4R)-4-(morpholin-4-yl)cyclohexyl]amino}- |
| | 1-(2,2,2-trifluoroethyl)-1H-indol-2-yl)prop-2-yn-1-yl]amino}benzene-1- |
| | sulfonamide |
| 888. | 2-{3-[(4-methanesulfonyl-2-methoxyphenyl)amino]prop-1-yn-1-yl}-N-[(1R,4R)- |
| | 4-(3-methanesulfonylazetidin-1-yl)cyclohexyl]-1-(2,2,2-trifluoroethyl)-1H-indol- |
| | 4-amine |
| 889. | 2-(3-((2-methoxy-4-(methylsulfonyl)phenyl)amino)prop-1-yn-1-yl)-N-((1S,4S)-4- |
| | (3-(methylsulfonyl)azetidin-1-yl)cyclohexyl)-1-(2,2,2-trifluoroethyl)-1H-indol-4- |
| | amine |
| 890. | 3-methoxy-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzene-1-sulfonamide |
| 891. | 3-methoxy-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzenesulfonamide |
| 892. | 3-methoxy-N-methyl-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzamide |
| 893. | 3-methoxy-N-methyl-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzamide |
| 894. | 2-{3-[(4-methanesulfonyl-2-methoxyphenyl)amino]prop-1-yn-1-yl}-N-[(1R,4R)- |
| | 4-{2-azaspiro[3.3]heptan-2-yl}cyclohexyl]-1-(2,2,2-trifluoroethyl)-1H-indol-4- |
| | amine |
| 895. | N-((1S,4S)-4-(2-azaspiro[3.3]heptan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 896. | 3-methoxy-N-methyl-4-{[3-(4-{[(1R,4R)-4-{2-azaspiro[3.3]heptan-2- |
| | yl}cyclohexyl]amino}-1-(2,2,2-trifluoroethyl)-1H-indol-2-yl)prop-2-yn-1- |
| | yl]amino}benzamide |
| 897. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxy-N- |
| | methylbenzamide |
| 898. | 3-methoxy-4-{[3-(4-{[(1R,4R)-4-{2-azaspiro[3.3]heptan-2-yl}cyclohexyl]amino}- |
| | 1-(2,2,2-trifluoroethyl)-1H-indol-2-yl)prop-2-yn-1-yl]amino}benzene-1- |
| | sulfonamide |
| 899. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3- |
| | methoxybenzenesulfonamide |
| 900. | 2-(3-((2-methoxy-4-(methylsulfonyl)phenyl)amino)prop-1-yn-1-yl)-N-(2- |
| | azaspiro[3.3]heptan-6-yl)-1-(2,2,2-trifluoroethyl)-1H-indol-4-amine |
| 901. | 4-((3-(4-((2-azaspiro[3.3]heptan-6-yl)amino)-1-(2,2,2-trifluoroethyl)-1H-indol-2- |
| | yl)prop-2-yn-1-yl)amino)-3-methoxybenzenesulfonamide |
| 902. | 2-{3-[(4-methanesulfonyl-2-methoxyphenyl)amino]prop-1-yn-1-yl}-N-[(1R,4R)- |
| | 4-{2-oxa-6-azaspiro[3.3]heptan-6-yl}cyclohexyl]-1-(2,2,2-trifluoroethyl)-1H- |
| | indol-4-amine |
| 903. | 2-{3-[(4-methanesulfonyl-2-methoxyphenyl)amino]prop-1-yn-1-yl}-N-[(1S,4S)-4- |
| | {2-oxa-6-azaspiro[3.3]heptan-6-yl}cyclohexyl]-1-(2,2,2-trifluoroethyl)-1H-indol- |
| | 4-amine |
| 904. | 3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 905. | 3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 906. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 907. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 908. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 909. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 910. | N-((1R,4R)-4-(2-oxa-6-azaspiro[3.3]heptan-6-yl)cyclohexyl)-2-(3-((2-(2- |
| | fluoroethoxy)-4-(methylsulfonyl)phenyl)amino)prop-1-yn-1-yl)-1-(2,2,2- |
| | trifluoroethyl)-1H-indol-4-amine |
| 911. | N-((1S,4S)-4-(2-oxa-6-azaspiro[3.3]heptan-6-yl)cyclohexyl)-2-(3-((2-(2- |
| | fluoroethoxy)-4-(methylsulfonyl)phenyl)amino)prop-1-yn-1-yl)-1-(2,2,2- |
| | trifluoroethyl)-1H-indol-4-amine |
| 912. | 3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 913. | 3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 914. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 915. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 916. | N-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)propionamide |
| 917. | N-(3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)propionamide |
| 918. | N-(2-hydroxy-4-methanesulfonylphenyl)-2-methyl-N-[3-(4-{[(1R,4R)-4-{2-oxa- |
| | 6-azaspiro[3.3]heptan-6-yl}cyclohexyl]amino}-1-(2,2,2-trifluoroethyl)-1H-indol- |
| | 2-yl)prop-2-yn-1-yl]propanamide |
| 919. | N-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)isobutyramide |
| 920. | 2-(3-{[4-methanesulfonyl-2-(2-methoxyethoxy)phenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 921. | 2-(3-{[4-methanesulfonyl-2-(2-methoxyethoxy)phenyl]amino}prop-1-yn-1-yl)-N- |
| | [(1S,4S)-4-{2-oxa-6-azaspiro[3.3]heptan-6-yl}cyclohexyl]-1-(2,2,2- |
| | trifluoroethyl)-1H-indol-4-amine |
| 922. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 923. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 924. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 925. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 926. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 927. | N-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)acetamide |
| 928. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 929. | 3-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 930. | 3-methoxy-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 931. | 2-(3-{[4-methanesulfonyl-2-(2,2,2-trifluoroethoxy)phenyl]amino}prop-1-yn-1-yl)- |
| | N-[(1R,4R)-4-{2-oxa-6-azaspiro[3.3]heptan-6-yl}cyclohexyl]-1-(2,2,2- |
| | trifluoroethyl)-1H-indol-4-amine |
| 932. | 2-(3-{[4-methanesulfonyl-2-(2,2,2-trifluoroethoxy)phenyl]amino}prop-1-yn-1-yl)- |
| | N-[(1S,4S)-4-{2-oxa-6-azaspiro[3.3]heptan-6-yl}cyclohexyl]-1-(2,2,2- |
| | trifluoroethyl)-1H-indol-4-amine |
| 933. | 2-[3-(2-amino-4-methanesulfonylphenoxy)prop-1-yn-1-yl]-N-[(1S,4S)-4-{2-oxa- |
| | 6-azaspiro[3.3]heptan-6-yl}cyclohexyl]-1-(2,2,2-trifluoroethyl)-1H-indol-4-amine |
| 934. | 2-{3-[(2-ethoxy-4-methanesulfonylphenyl)amino]prop-1-yn-1-yl}-N-[(1R,4R)-4- |
| | {2-oxa-6-azaspiro[3.3]heptan-6-yl}cyclohexyl]-1-(2,2,2-trifluoroethyl)-1H-indol- |
| | 4-amine |
| 935. | 2-{3-[(2-ethoxy-4-methanesulfonylphenyl)amino]prop-1-yn-1-yl}-N-[(1S,4S)-4- |
| | {2-oxa-6-azaspiro[3.3]heptan-6-yl}cyclohexyl]-1-(2,2,2-trifluoroethyl)-1H-indol- |
| | 4-amine |
| 936. | 3-(2-methoxyethoxy)-N-methyl-4-{[3-(4-{[(1R,4R)-4-{2-oxa-6- |
| | azaspiro[3.3]heptan-6-yl}cyclohexyl]amino}-1-(2,2,2-trifluoroethyl)-1H-indol-2- |
| | yl)prop-2-yn-1-yl]amino}benzamide |
| 937. | 3-(2-methoxyethoxy)-N-methyl-4-{[3-(4-{[(1S,4S)-4-{2-oxa-6- |
| | azaspiro[3.3]heptan-6-yl}cyclohexyl]amino}-1-(2,2,2-trifluoroethyl)-1H-indol-2- |
| | yl)prop-2-yn-1-yl]amino}benzamide |
| 938. | N-ethyl-3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 939. | N-ethyl-3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 940. | 3-(2-fluoroethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 941. | 3-(2-fluoroethoxy)-N-methyl-4-{[3-(4-{[(1S,4S)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 942. | 3-(cyanomethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 943. | 3-(cyanomethoxy)-N-methyl-4-{[3-(4-{[(1S,4S)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 944. | 2-{3-[4-methanesulfonyl-2-(methylamino)phenoxy]prop-1-yn-1-yl}-N-[(1S,4S)-4- |
| | {2-oxa-6-azaspiro[3.3]heptan-6-yl}cyclohexyl]-1-(2,2,2-trifluoroethyl)-1H-indol- |
| | 4-amine |
| 945. | 2-{3-[4-methanesulfonyl-2-(methylamino)phenoxy]prop-1-yn-1-yl}-N-[(1R,4R)- |
| | 4-{2-oxa-6-azaspiro[3.3]heptan-6-yl}cyclohexyl]-1-(2,2,2-trifluoroethyl)-1H- |
| | indol-4-amine |
| 946. | 2-[3-(2-amino-4-methanesulfonylphenoxy)prop-1-yn-1-yl]-N-[(1R,4R)-4-{2-oxa- |
| | 6-azaspiro[3.3]heptan-6-yl}cyclohexyl]-1-(2,2,2-trifluoroethyl)-1H-indol-4-amine |
| 947. | N-((1R,4R)-4-(2-oxa-6-azaspiro[3.3]heptan-6-yl)cyclohexyl)-2-(3-(2-amino-4- |
| | (methylsulfonyl)phenoxy)prop-1-yn-1-yl)-1-(2,2,2-trifluoroethyl)-1H-indol-4- |
| | amine |
| 948. | 3-hydroxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 949. | 3-methoxy-4-[(3-{4-[(4-{2-oxa-6-azaspiro[3.3]heptan-6-yl}cyclohexyl)amino]-1- |
| | (2,2,2-trifluoroethyl)-1H-indol-2-yl}prop-2-yn-1-yl)amino]benzonitrile |
| 950. | 3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzonitrile |
| 951. | 2-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 952. | 2-(4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxyphenyl)-2- |
| | methylpropanenitrile |
| 953. | 3-(fluoromethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 954. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-(fluoromethoxy)-N- |
| | methylbenzamide |
| 955. | 3-(2-cyanoethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 956. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-(2-cyanoethoxy)-N- |
| | methylbenzamide |
| 957. | 2-(3-{[2-(fluoromethoxy)-4-methanesulfonylphenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 958. | N-((1S,4S)-4-(2-oxa-6-azaspiro[3.3]heptan-6-yl)cyclohexyl)-2-(3-((2- |
| | (fluoromethoxy)-4-(methylsulfonyl)phenyl)amino)prop-1-yn-1-yl)-1-(2,2,2- |
| | trifluoroethyl)-1H-indol-4-amine |
| 959. | 3-(cyanomethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 960. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3- |
| | (cyanomethoxy)benzenesulfonamide |
| 961. | 3-(fluoromethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 962. | 3-(fluoromethoxy)-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 963. | 4-((3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 964. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 965. | N-((1R,4R)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 966. | N-((1S,4S)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 967. | 4-((3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N,N- |
| | dimethylbenzenesulfonamide |
| 968. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N,N- |
| | dimethylbenzenesulfonamide |
| 969. | N-((1R,4R)-4-(2-oxa-6-azaspiro[3.3]heptan-6-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 970. | N-((1S,4S)-4-(2-oxa-6-azaspiro[3.3]heptan-6-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 971. | N-((1R,4R)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)- |
| | 1H-indol-4-amine |
| 972. | N-((1S,4S)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)- |
| | 1H-indol-4-amine |
| 973. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 974. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-2-fluoro-5- |
| | methoxybenzamide |
| 975. | 2-fluoro-5-methoxy-N-methyl-4-{[3-(4-{[(1R,4R)-4-{2-oxa-6- |
| | azaspiro[3.3]heptan-6-yl}cyclohexyl]amino}-1-(2,2,2-trifluoroethyl)-1H-indol-2- |
| | yl)prop-2-yn-1-yl]amino}benzamide |
| 976. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-2-fluoro-5-methoxy- |
| | N-methylbenzamide |
| 977. | 2-(3-{[2-methoxy-4-(trifluoromethyl)phenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H- |
| | indol-4-amine |
| 978. | N-((1S,4S)-4-(2-oxa-6-azaspiro[3.3]heptan-6-yl)cyclohexyl)-2-(3-((2-methoxy-4- |
| | (trifluoromethyl)phenyl)amino)prop-1-yn-1-yl)-1-(2,2,2-trifluoroethyl)-1H-indol- |
| | 4-amine |
| 979. | 4-((3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxybenzoic |
| | acid |
| 980. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxybenzoic |
| | acid |
| 981. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzoic acid |
| 982. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-2-fluoro-5- |
| | methoxybenzoic acid |
| 983. | 2-{3-[(4-methanesulfonyl-2-methylphenyl)amino]prop-1-yn-1-yl}-N-[(1R,4R)-4- |
| | {2-oxa-6-azaspiro[3.3]heptan-6-yl}cyclohexyl]-1-(2,2,2-trifluoroethyl)-1H-indol- |
| | 4-amine |
| 984. | N-((1S,4S)-4-(2-oxa-6-azaspiro[3.3]heptan-6-yl)cyclohexyl)-2-(3-((2-methyl-4- |
| | (methylsulfonyl)phenyl)amino)prop-1-yn-1-yl)-1-(2,2,2-trifluoroethyl)-1H-indol- |
| | 4-amine |
| 985. | 2-(3-{[4-methanesulfonyl-2-(trifluoromethoxy)phenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 986. | N-((1S,4S)-4-(2-oxa-6-azaspiro[3.3]heptan-6-yl)cyclohexyl)-2-(3-((4- |
| | (methylsulfonyl)-2-(trifluoromethoxy)phenyl)amino)prop-1-yn-1-yl)-1-(2,2,2- |
| | trifluoroethyl)-1H-indol-4-amine |
| 987. | 2-(3-{[4-methanesulfonyl-2-(trifluoromethyl)phenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 988. | N-((1S,4S)-4-(2-oxa-6-azaspiro[3.3]heptan-6-yl)cyclohexyl)-2-(3-((4- |
| | (methylsulfonyl)-2-(trifluoromethyl)phenyl)amino)prop-1-yn-1-yl)-1-(2,2,2- |
| | trifluoroethyl)-1H-indol-4-amine |
| 989. | 2-{3-[(2-chloro-4-methanesulfonylphenyl)amino]prop-1-yn-1-yl}-N-[(1R,4R)-4- |
| | {2-oxa-6-azaspiro[3.3]heptan-6-yl}cyclohexyl]-1-(2,2,2-trifluoroethyl)-1H-indol- |
| | 4-amine |
| 990. | N-((1S,4S)-4-(2-oxa-6-azaspiro[3.3]heptan-6-yl)cyclohexyl)-2-(3-((2-chloro-4- |
| | (methylsulfonyl)phenyl)amino)prop-1-yn-1-yl)-1-(2,2,2-trifluoroethyl)-1H-indol- |
| | 4-amine |
| 991. | 3-(3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-6- |
| | (methylsulfonyl)benzo[d]oxazol-2(3H)-one |
| 992. | 3-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-6- |
| | (methylsulfonyl)benzo[d]oxazol-2(3H)-one |
| 993. | 2-{3-[(4-methanesulfonyl-2-methoxyphenyl)amino]prop-1-yn-1-yl}-N-[(1S,4S)-4- |
| | {7-oxa-2-azaspiro[3.5]nonan-2-yl}cyclohexyl]-1-(2,2,2-trifluoroethyl)-1H-indol- |
| | 4-amine |
| 994. | 2-{3-[(4-methanesulfonyl-2-methoxyphenyl)amino]prop-1-yn-1-yl}-N-[(1R,4R)- |
| | 4-{7-oxa-2-azaspiro[3.5]nonan-2-yl}cyclohexyl]-1-(2,2,2-trifluoroethyl)-1H- |
| | indol-4-amine |
| 995. | 3-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 996. | 3-methoxy-N-methyl-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 997. | 3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 998. | 3-methoxy-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 999. | 3-(cyanomethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1000. | 3-(cyanomethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1001. | 2-(3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1002. | 2-(4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxyphenyl)-2- |
| | methylpropanenitrile |
| 1003. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1004. | 2-(5-methanesulfonyl-2-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1005. | N-((4-((3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)acetamide |
| 1006. | N-((4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)acetamide |
| 1007. | N-((4-((3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)-2-aminoacetamide |
| 1008. | N-((4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)-2-aminoacetamide |
| 1009. | methyl 2-(5-methanesulfonyl-2-{[3-(4-{[(1R,4R)-4-{7-oxa-2-azaspiro[3.5]nonan- |
| | 2-yl}cyclohexyl]amino}-1-(2,2,2-trifluoroethyl)-1H-indol-2-yl)prop-2-yn-1- |
| | yl]amino}phenoxy)acetate |
| 1010. | methyl 2-(2-((3-(4-(((1S,4S)-4-(7-oxa-2-azaspiro[3.5]nonan-2- |
| | yl)cyclohexyl)amino)-1-(2,2,2-trifluoroethyl)-1H-indol-2-yl)prop-2-yn-1- |
| | yl)amino)-5-(methylsulfonyl)phenoxy)acetate |
| 1011. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetic acid |
| 1012. | 2-(2-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetic acid |
| 1013. | 2-{3-[(4-methanesulfonyl-2-methoxyphenyl)amino]prop-1-yn-1-yl}-N-[(1R,4R)- |
| | 4-{2-oxa-7-azaspiro[3.5]nonan-7-yl}cyclohexyl]-1-(2,2,2-trifluoroethyl)-1H- |
| | indol-4-amine |
| 1014. | 2-(3-{[4-(ethanesulfonyl)-2-methoxyphenyl]amino}prop-1-yn-1-yl)-N-[(1R,4R)- |
| | 4-{7-oxa-2-azaspiro[3.5]nonan-2-yl}cyclohexyl]-1-(2,2,2-trifluoroethyl)-1H- |
| | indol-4-amine |
| 1015. | 2-(3-{[4-(ethanesulfonyl)-2-methoxyphenyl]amino}prop-1-yn-1-yl)-N-[(1S,4S)-4- |
| | {7-oxa-2-azaspiro[3.5]nonan-2-yl}cyclohexyl]-1-(2,2,2-trifluoroethyl)-1H-indol- |
| | 4-amine |
| 1016. | 3-(fluoromethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1017. | 3-(fluoromethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1018. | 3-methoxy-N-methyl-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1019. | 4-((3-(4-(((1S,4S)-4-(6-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxy-N- |
| | methylbenzamide |
| 1020. | 3-methoxy-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1021. | 4-((3-(4-(((1S,4S)-4-(6-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1022. | 2-{3-[(4-methanesulfonyl-2-methoxyphenyl)amino]prop-1-yn-1-yl}-N-[(1R,4R)- |
| | 4-{6-oxa-2-azaspiro[3.5]nonan-2-yl}cyclohexyl]-1-(2,2,2-trifluoroethyl)-1H- |
| | indol-4-amine |
| 1023. | N-((1S,4S)-4-(6-oxa-2-azaspiro[3.5]nonan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1024. | N-((1R,4R)-4-(7-oxa-2-azaspiro[3.5]nonan-2-yl)cyclohexyl)-2-(3-((2- |
| | (fluoromethoxy)-4-(methylsulfonyl)phenyl)amino)prop-1-yn-1-yl)-1-(2,2,2- |
| | trifluoroethyl)-1H-indol-4-amine |
| 1025. | N-((1S,4S)-4-(7-oxa-2-azaspiro[3.5]nonan-2-yl)cyclohexyl)-2-(3-((2- |
| | (fluoromethoxy)-4-(methylsulfonyl)phenyl)amino)prop-1-yn-1-yl)-1-(2,2,2- |
| | trifluoroethyl)-1H-indol-4-amine |
| 1026. | 4-(3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)-7-(methylsulfonyl)-2H- |
| | benzo[b][1,4]oxazin-3(4H)-one |
| 1027. | 4-((3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 1028. | 4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 1029. | N-((1R,4R)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1030. | N-((1S,4S)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1031. | 3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1032. | 4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxybenzamide |
| 1033. | 3-methoxy-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1034. | 4-((3-(4-(((1S,4S)-4-(6-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxybenzamide |
| 1035. | 2-fluoro-5-methoxy-N-methyl-4-{[3-(4-{[(1R,4R)-4-{7-oxa-2- |
| | azaspiro[3.5]nonan-2-yl}cyclohexyl]amino}-1-(2,2,2-trifluoroethyl)-1H-indol-2- |
| | yl)prop-2-yn-1-yl]amino}benzamide |
| 1036. | 2-(3-((2-methoxy-4-(methylsulfonyl)phenyl)amino)prop-1-yn-1-yl)-N-((1R,4R)-4- |
| | (3-methoxypyrrolidin-1-yl)cyclohexyl)-1-(2,2,2-trifluoroethyl)-1H-indol-4-amine |
| 1037. | 2-(3-((2-methoxy-4-(methylsulfonyl)phenyl)amino)prop-1-yn-1-yl)-N-((1S,4S)-4- |
| | (3-methoxypyrrolidin-1-yl)cyclohexyl)-1-(2,2,2-trifluoroethyl)-1H-indol-4-amine |
| 1038. | 3-methoxy-4-((3-(4-(((1R,4R)-4-(3-methoxypyrrolidin-1-yl)cyclohexyl)amino)-1- |
| | (2,2,2-trifluoroethyl)-1H-indol-2-yl)prop-2-yn-1-yl)amino)benzenesulfonamide |
| 1039. | 3-methoxy-4-((3-(4-(((1S,4S)-4-(3-methoxypyrrolidin-1-yl)cyclohexyl)amino)-1- |
| | (2,2,2-trifluoroethyl)-1H-indol-2-yl)prop-2-yn-1-yl)amino)benzenesulfonamide |
| 1040. | 2-(3-{[2-(fluoromethoxy)-4-methanesulfonylphenyl]amino}prop-1-yn-1-yl)-N- |
| | [(1R,4R)-4-(morpholin-4-yl)cyclohexyl]-1-(2,2,2-trifluoroethyl)-1H-indol-4- |
| | amine |
| 1041. | 2-(3-{[2-(fluoromethoxy)-4-methanesulfonylphenyl]amino}prop-1-yn-1-yl)-N- |
| | [(1S,4S)-4-(morpholin-4-yl)cyclohexyl]-1-(2,2,2-trifluoroethyl)-1H-indol-4-amine |
| 1042. | 3-(cyanomethoxy)-4-{[3-(4-{[(1R,4R)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1043. | 3-(cyanomethoxy)-4-{[3-(4-{[(1S,4S)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1044. | 3-(fluoromethoxy)-4-{[3-(4-{[(1R,4R)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1045. | 3-(fluoromethoxy)-4-{[3-(4-{[(1S,4S)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1046. | 2-(3-((2-ethoxy-4-(methylsulfonyl)phenyl)amino)prop-1-yn-1-yl)-N-((1R,4R)-4- |
| | morpholinocyclohexyl)-1-(2,2,2-trifluoroethyl)-1H-indol-4-amine |
| 1047. | 2-(3-((2-ethoxy-4-(methylsulfonyl)phenyl)amino)prop-1-yn-1-yl)-N-((1S,4S)-4- |
| | morpholinocyclohexyl)-1-(2,2,2-trifluoroethyl)-1H-indol-4-amine |
| 1048. | 2-{3-[(4-methanesulfonyl-2-methoxyphenyl)amino]prop-1-yn-1-yl}-N-[(1R,4R)- |
| | 4-(3-methoxypiperidin-1-yl)cyclohexyl]-1-(2,2,2-trifluoroethyl)-1H-indol-4-amine |
| 1049. | 2-(3-((2-methoxy-4-(methylsulfonyl)phenyl)amino)prop-1-yn-1-yl)-N-((1S,4S)-4- |
| | (3-methoxypiperidin-1-yl)cyclohexyl)-1-(2,2,2-trifluoroethyl)-1H-indol-4-amine |
| 1050. | 2-{3-[(4-methanesulfonyl-2-methoxyphenyl)amino]prop-1-yn-1-yl}-N-[(1R,4R)- |
| | 4-(4-methoxypiperidin-1-yl)cyclohexyl]-1-(2,2,2-trifluoroethyl)-1H-indol-4-amine |
| 1051. | 2-(3-((2-methoxy-4-(methylsulfonyl)phenyl)amino)prop-1-yn-1-yl)-N-((1S,4S)-4- |
| | (4-methoxypiperidin-1-yl)cyclohexyl)-1-(2,2,2-trifluoroethyl)-1H-indol-4-amine |
| 1052. | 3-methoxy-N-methyl-4-{[3-(4-{[(1R,4R)-4-(4-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1053. | 3-methoxy-4-((3-(4-(((1S,4S)-4-(4-methoxypiperidin-1-yl)cyclohexyl)amino)-1- |
| | (2,2,2-trifluoroethyl)-1H-indol-2-yl)prop-2-yn-1-yl)amino)-N-methylbenzamide |
| 1054. | 3-methoxy-4-{[3-(4-{[(1R,4R)-4-(4-methoxypiperidin-1-yl)cyclohexyl]amino}-1- |
| | (2,2,2-trifluoroethyl)-1H-indol-2-yl)prop-2-yn-1-yl]amino}benzene-1-sulfonamide |
| 1055. | 3-methoxy-4-((3-(4-(((1S,4S)-4-(4-methoxypiperidin-1-yl)cyclohexyl)amino)-1- |
| | (2,2,2-trifluoroethyl)-1H-indol-2-yl)prop-2-yn-1-yl)amino)benzenesulfonamide |
| 1056. | 3-methoxy-N-methyl-4-{[3-(4-{[(1R,4R)-4-(3-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1057. | 3-methoxy-4-((3-(4-(((1S,4S)-4-(3-methoxypiperidin-1-yl)cyclohexyl)amino)-1- |
| | (2,2,2-trifluoroethyl)-1H-indol-2-yl)prop-2-yn-1-yl)amino)-N-methylbenzamide |
| 1058. | 3-methoxy-4-{[3-(4-{[(1R,4R)-4-(3-methoxypiperidin-1-yl)cyclohexyl]amino}-1- |
| | (2,2,2-trifluoroethyl)-1H-indol-2-yl)prop-2-yn-1-yl]amino}benzene-1-sulfonamide |
| 1059. | 3-methoxy-4-((3-(4-(((1S,4S)-4-(3-methoxypiperidin-1-yl)cyclohexyl)amino)-1- |
| | (2,2,2-trifluoroethyl)-1H-indol-2-yl)prop-2-yn-1-yl)amino)benzenesulfonamide |
| 1060. | 3-methoxy-4-((3-(4-(((1R,4R)-4-morpholinocyclohexyl)amino)-1-(2,2,2- |
| | trifluoroethyl)-1H-indol-2-yl)prop-2-yn-1-yl)amino)benzenesulfonamide |
| 1061. | 3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzenesulfonamide |
| 1062. | 4-[(1R,4R)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-1λ⁶-thiomorpholine- |
| | 1,1-dione |
| 1063. | 4-((1S,4S)-4-((2-(3-((2-methoxy-4-(methylsulfonyl)phenyl)amino)prop-1-yn-1- |
| | yl)-1-(2,2,2-trifluoroethyl)-1H-indol-4-yl)amino)cyclohexyl)thiomorpholine 1,1- |
| | dioxide |
| 1064. | 4-[(1R,4R)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-124-thiomorpholin- |
| | 1-one |
| 1065. | 4-[(1S,4S)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-124-thiomorpholin- |
| | 1-one |
| 1066. | 4-((3-(4-(((1R,4R)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N- |
| | methylbenzamide |
| 1067. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N- |
| | methylbenzamide |
| 1068. | 4-((1S,4S)-4-((2-(3-((2-methoxy-4-(methylsulfonyl)phenyl)amino)prop-1-yn-1- |
| | yl)-1-(2,2,2-trifluoroethyl)-1H-indol-4-yl)amino)cyclohexyl)thiomorpholine 1- |
| | oxide |
| 1069. | 3-methoxy-N-methyl-4-{[3-(4-{[(1R,4R)-4-(1-oxo-124-thiomorpholin-4- |
| | yl)cyclohexyl]amino}-1-(2,2,2-trifluoroethyl)-1H-indol-2-yl)prop-2-yn-1- |
| | yl]amino}benzamide |
| 1070. | 3-methoxy-4-{[3-(4-{[(1R,4R)-4-(1-oxo-12.4-thiomorpholin-4- |
| | yl)cyclohexyl]amino}-1-(2,2,2-trifluoroethyl)-1H-indol-2-yl)prop-2-yn-1- |
| | yl]amino}benzene-1-sulfonamide |
| 1071. | 3-methoxy-4-((3-(4-(((1S,4S)-4-(1-oxidothiomorpholino)cyclohexyl)amino)-1- |
| | (2,2,2-trifluoroethyl)-1H-indol-2-yl)prop-2-yn-1-yl)amino)benzenesulfonamide |
| 1072. | 3-methoxy-4-{[3-(4-{[(1R,4R)-4-(1,1-dioxo-1λ⁶-thiomorpholin-4- |
| | yl)cyclohexyl]amino}-1-(2,2,2-trifluoroethyl)-1H-indol-2-yl)prop-2-yn-1- |
| | yl]amino}benzene-1-sulfonamide |
| 1073. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxybenzenesulfonamide |
| 1074. | 2-{3-[(4-methanesulfonyl-2-methoxyphenyl)amino]prop-1-yn-1-yl}-N-[(1S,4S)-4- |
| | {2-oxa-7-azaspiro[3.5]nonan-7-yl}cyclohexyl]-1-(2,2,2-trifluoroethyl)-1H-indol- |
| | 4-amine |
| 1075. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1076. | 3-methoxy-N-methyl-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1077. | 3-methoxy-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1078. | 4-((3-(4-(((1S,4S)-4-(2-oxa-7-azaspiro[3.5]nonan-7-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1079. | 3-(cyanomethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1080. | 3-(cyanomethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1081. | 3-(fluoromethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1082. | 3-(fluoromethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1083. | 2-{3-[(4-methanesulfonyl-2-methoxyphenyl)amino]prop-1-yn-1-yl}-N-[(1R,4R)- |
| | 4-{1-oxa-7-azaspiro[3.5]nonan-7-yl}cyclohexyl]-1-(2,2,2-trifluoroethyl)-1H- |
| | indol-4-amine |
| 1084. | N-((1S,4S)-4-(1-oxa-7-azaspiro[3.5]nonan-7-yl)cyclohexyl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1085. | 3-methoxy-N-methyl-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1086. | 4-((3-(4-(((1S,4S)-4-(1-oxa-7-azaspiro[3.5]nonan-7-yl)cyclohexyl)amino)-1- |
| | (2,2,2-trifluoroethyl)-1H-indol-2-yl)prop-2-yn-1-yl)amino)-3-methoxy-N- |
| | methylbenzamide |
| 1087. | 3-methoxy-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1088. | 4-((3-(4-(((1S,4S)-4-(1-oxa-7-azaspiro[3.5]nonan-7-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1089. | 2-(5-methanesulfonyl-2-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1090. | 2-(5-methanesulfonyl-2-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1091. | 2-(3-{[2-(fluoromethoxy)-4-methanesulfonylphenyl]amino}prop-1-yn-1-yl)-N- |
| | [(1R,4R)-4-{2-oxa-7-azaspiro[3.5]nonan-7-yl}cyclohexyl]-1-(2,2,2- |
| | trifluoroethyl)-1H-indol-4-amine |
| 1092. | 2-(3-{[2-(fluoromethoxy)-4-methanesulfonylphenyl]amino}prop-1-yn-1-yl)-N- |
| | [(1S,4S)-4-{2-oxa-7-azaspiro[3.5]nonan-7-yl}cyclohexyl]-1-(2,2,2-trifluoroethyl)- |
| | 1H-indol-4-amine |
| 1093. | N-((1R,4R)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1094. | N-((1S,4S)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1095. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1096. | N-((1R,4R)-4-(2-oxa-7-azaspiro[3.5]nonan-7-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 1097. | N-((1S,4S)-4-(2-oxa-7-azaspiro[3.5]nonan-7-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 1098. | 2-{3-[(4-methanesulfonyl-2-methoxyphenyl)amino]prop-1-yn-1-yl}-N-[(1R,4R)- |
| | 4-{6-oxa-3-azabicyclo[3.1.1]heptan-3-yl}cyclohexyl]-1-(2,2,2-trifluoroethyl)-1H- |
| | indol-4-amine |
| 1099. | 2-{3-[(4-methanesulfonyl-2-methoxyphenyl)amino]prop-1-yn-1-yl}-N-[(1S,4S)-4- |
| | {6-oxa-3-azabicyclo[3.1.1]heptan-3-yl}cyclohexyl]-1-(2,2,2-trifluoroethyl)-1H- |
| | indol-4-amine |
| 1100. | 3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1101. | 3-methoxy-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1102. | 3-methoxy-N-methyl-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1103. | 3-methoxy-N-methyl-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1104. | 2-{3-[(2-ethoxy-4-methanesulfonylphenyl)amino]prop-1-yn-1-yl}-N-[(1R,4R)-4- |
| | {6-oxa-3-azabicyclo[3.1.1]heptan-3-yl}cyclohexyl]-1-(2,2,2-trifluoroethyl)-1H- |
| | indol-4-amine |
| 1105. | 2-{3-[(2-ethoxy-4-methanesulfonylphenyl)amino]prop-1-yn-1-yl}-N-[(1S,4S)-4- |
| | {6-oxa-3-azabicyclo[3.1.1]heptan-3-yl}cyclohexyl]-1-(2,2,2-trifluoroethyl)-1H- |
| | indol-4-amine |
| 1106. | N-ethyl-3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1107. | 2-(5-methanesulfonyl-2-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}phenoxy)acetonitrile |
| 1108. | 2-(2-((3-(4-(((1S,4S)-4-(6-oxa-3-azabicyclo[3.1.1]heptan-3-yl)cyclohexyl)amino)- |
| | 1-(2,2,2-trifluoroethyl)-1H-indol-2-yl)prop-2-yn-1-yl)amino)-5- |
| | (methylsulfonyl)phenoxy)acetonitrile |
| 1109. | 3-methoxy-N-methyl-4-{[3-(4-{[(1R,4R)-4-{2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzamide |
| 1110. | 2-{3-[(4-methanesulfonyl-2-methoxyphenyl)amino]prop-1-yn-1-yl}-N-[(1R,4R)- |
| | 4-{2-oxa-8-azaspiro[4.5]decan-8-yl}cyclohexyl]-1-(2,2,2-trifluoroethyl)-1H- |
| | indol-4-amine |
| 1111. | 3-methoxy-4-{[3-(4-{[(1R,4R)-4-{2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzene-1-sulfonamide |
| 1112. | 4-((3-(4-(((1R,4R)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxybenzenesulfonamide |
| 1113. | 4-((3-(4-(((1S,4S)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxybenzenesulfonamide |
| 1114. | 2-{3-[(4-methanesulfonyl-2-methoxyphenyl)amino]prop-1-yn-1-yl}-N-[(1R,4R)- |
| | 4-{3-oxa-9-azaspiro[5.5]undecan-9-yl}cyclohexyl]-1-(2,2,2-trifluoroethyl)-1H- |
| | indol-4-amine |
| 1115. | N-((1S,4S)-4-(3-oxa-9-azaspiro[5.5]undecan-9-yl)cyclohexyl)-2-(3-((2-methoxy- |
| | 4-(methylsulfonyl)phenyl)amino)prop-1-yn-1-yl)-1-(2,2,2-trifluoroethyl)-1H- |
| | indol-4-amine |
| 1116. | 3-methoxy-N-methyl-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzamide |
| 1117. | 4-((3-(4-(((1S,4S)-4-(3-oxa-9-azaspiro[5.5]undecan-9-yl)cyclohexyl)amino)-1- |
| | (2,2,2-trifluoroethyl)-1H-indol-2-yl)prop-2-yn-1-yl)amino)-3-methoxy-N- |
| | methylbenzamide |
| 1118. | 3-methoxy-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzene-1-sulfonamide |
| 1119. | 4-((3-(4-(((1S,4S)-4-(3-oxa-9-azaspiro[5.5]undecan-9-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1120. | 3-methoxy-N-methyl-4-{[3-(4-{[(1R,4R)-4-(pyrrolidin-1-yl)cyclohexyl]amino}-1- |
| | (2,2,2-trifluoroethyl)-1H-indol-2-yl)prop-2-yn-1-yl]amino}benzamide |
| 1121. | 3-methoxy-N-methyl-4-((3-(4-(((1S,4S)-4-(pyrrolidin-1-yl)cyclohexyl)amino)-1- |
| | (2,2,2-trifluoroethyl)-1H-indol-2-yl)prop-2-yn-1-yl)amino)benzamide |
| 1122. | 3-methoxy-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1123. | 3-methoxy-4-((3-(4-(((1S,4S)-4-(pyrrolidin-1-yl)cyclohexyl)amino)-1-(2,2,2- |
| | trifluoroethyl)-1H-indol-2-yl)prop-2-yn-1-yl)amino)benzenesulfonamide |
| 1124. | 2-{3-[(4-methanesulfonyl-2-methoxyphenyl)amino]prop-1-yn-1-yl}-N-[(1R,4R)- |
| | 4-(pyrrolidin-1-yl)cyclohexyl]-1-(2,2,2-trifluoroethyl)-1H-indol-4-amine |
| 1125. | 2-(3-((2-methoxy-4-(methylsulfonyl)phenyl)amino)prop-1-yn-1-yl)-N-((1S,4S)-4- |
| | (pyrrolidin-1-yl)cyclohexyl)-1-(2,2,2-trifluoroethyl)-1H-indol-4-amine |
| 1126. | -{3-[(4-methanesulfonyl-2-methoxyphenyl)amino]prop-1-yn-1-yl}-N-[(1R,4R)-4- |
| | (3-methoxypyrrolidin-1-yl)cyclohexyl]-1-(2,2,2-trifluoroethyl)-1H-indol-4-amine |
| 1127. | 3-methoxy-4-{[3-(4-{[(1R,4R)-4-(3-methoxypyrrolidin-1-yl)cyclohexyl]amino}- |
| | 1-(2,2,2-trifluoroethyl)-1H-indol-2-yl)prop-2-yn-1-yl]amino}benzene-1- |
| | sulfonamide |
| 1128. | 3-methoxy-N-methyl-4-{[3-(4-{[(1R,4R)-4-(3-methoxypyrrolidin-1- |
| | yl)cyclohexyl]amino}-1-(2,2,2-trifluoroethyl)-1H-indol-2-yl)prop-2-yn-1- |
| | yl]amino}benzamide |
| 1129. | 3-methoxy-4-((3-(4-(((1S,4S)-4-(3-methoxypyrrolidin-1-yl)cyclohexyl)amino)-1- |
| | (2,2,2-trifluoroethyl)-1H-indol-2-yl)prop-2-yn-1-yl)amino)-N-methylbenzamide |
| 1130. | 3-methoxy-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzene-1-sulfonamide |
| 1131. | 3-methoxy-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzenesulfonamide |
| 1132. | 2-{3-[(4-methanesulfonyl-2-methoxyphenyl)amino]prop-1-yn-1-yl}-N-[(1R,4R)- |
| | 4-{hexahydro-1H-furo[3,4-c]pyrrol-5-yl}cyclohexyl]-1-(2,2,2-trifluoroethyl)-1H- |
| | indol-4-amine |
| 1133. | 2-(3-((2-methoxy-4-(methylsulfonyl)phenyl)amino)prop-1-yn-1-yl)-N-((1R,4R)-4- |
| | (tetrahydro-1H-furo[3,4-c]pyrrol-5(3H)-yl)cyclohexyl)-1-(2,2,2-trifluoroethyl)- |
| | 1H-indol-4-amine |
| 1134. | 3-methoxy-N-methyl-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzamide |
| 1135. | 3-methoxy-N-methyl-4-((3-(4-(((1S,4S)-4-(tetrahydro-1H-furo[3,4-c]pyrrol- |
| | 5(3H)-yl)cyclohexyl)amino)-1-(2,2,2-trifluoroethyl)-1H-indol-2-yl)prop-2-yn-1- |
| | yl)amino)benzamide |
| 1136. | 2-{3-[(4-methanesulfonyl-2-methoxyphenyl)amino]prop-1-yn-1-yl}-N-[(1R,4R)- |
| | 4-[4-(trifluoromethyl)piperidin-1-yl]cyclohexyl]-1-(2,2,2-trifluoroethyl)-1H-indol- |
| | 4-amine |
| 1137. | 2-(3-((2-methoxy-4-(methylsulfonyl)phenyl)amino)prop-1-yn-1-yl)-1-(2,2,2- |
| | trifluoroethyl)-N-((1S,4S)-4-(4-(trifluoromethyl)piperidin-1-yl)cyclohexyl)-1H- |
| | indol-4-amine |
| 1138. | 3-methoxy-N-methyl-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzamide |
| 1139. | 3-methoxy-N-methyl-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzamide |
| 1140. | 3-methoxy-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 1141. | 3-methoxy-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzenesulfonamide |
| 1142. | 2-{3-[(4-methanesulfonyl-2-methoxyphenyl)amino]prop-1-yn-1-yl}-N-[(1R,4R)- |
| | 4-(4-methanesulfonylpiperidin-1-yl)cyclohexyl]-1-(2,2,2-trifluoroethyl)-1H-indol- |
| | 4-amine |
| 1143. | 2-(3-((2-methoxy-4-(methylsulfonyl)phenyl)amino)prop-1-yn-1-yl)-N-((1S,4S)-4- |
| | (4-(methylsulfonyl)piperidin-1-yl)cyclohexyl)-1-(2,2,2-trifluoroethyl)-1H-indol-4- |
| | amine |
| 1144. | 3-methoxy-N-methyl-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzamide |
| 1145. | 3-methoxy-N-methyl-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzamide |
| 1146. | 3-methoxy-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzene-1-sulfonamide |
| 1147. | 3-methoxy-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzenesulfonamide |
| 1148. | 1-[(1S,3R)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-4,5-dihydro-1H- |
| | 1,2,3,4-tetrazol-5-one |
| 1149. | 2-(3-((2-methoxy-4-(methylsulfonyl)phenyl)amino)prop-1-yn-1-yl)-N-((1R,4R)-4- |
| | morpholinocyclohexyl)-1-(oxiran-2-ylmethyl)-1H-indol-4-amine |
| 1150. | 2-((2-(3-((2-methoxy-4-(methylsulfonyl)phenyl)amino)prop-1-yn-1-yl)-4- |
| | (((1R,4R)-4-morpholinocyclohexyl)amino)-1H-indol-1-yl)methyl)acrylonitrile |
| 1151. | N-((1R,4R)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1152. | N-((1S,4S)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1153. | 4-((3-(4-(((1R,4R)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxy-N-methylbenzamide |
| 1154. | 4-((3-(4-(((1S,4S)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxy-N-methylbenzamide |
| 1155. | 1-[1-(2-hydroxyethyl)piperidin-4-yl]-3-(2-{3-[(4-methanesulfonyl-2- |
| | methoxyphenyl)amino]prop-1-yn-1-yl}-1-(2,2,2-trifluoroethyl)-1H-indol-4- |
| | yl)urea |
| 1156. | 3-methoxy-4-({3-[4-({2-oxaspiro[3.3]heptan-6-yl}amino)-1-(2,2,2-trifluoroethyl)- |
| | 1H-indol-2-yl]prop-2-yn-1-yl}amino)benzene-1-sulfonamide |
| 1157. | [1-(chloromethyl)-3-[(2-{3-[(4-methanesulfonyl-2-methoxyphenyl)amino]prop-1- |
| | yn-1-yl}-1-(2,2,2-trifluoroethyl)-1H-indol-4-yl)amino]cyclobutyl]methanol |
| 1158. | 2-{3-[(4-methanesulfonyl-2-methoxyphenyl)amino]prop-1-yn-1-yl}-N-{2- |
| | oxaspiro[3.3]heptan-6-yl}-1-(2,2,2-trifluoroethyl)-1H-indol-4-amine |
| 1159. | 4-({3-[4-({2-azaspiro[3.3]heptan-6-yl}amino)-1-(2,2,2-trifluoroethyl)-1H-indol-2- |
| | yl]prop-2-yn-1-yl}amino)-3-methoxybenzene-1-sulfonamide |
| 1160. | N-{2-azaspiro[3.3]heptan-6-yl}-2-{3-[(4-methanesulfonyl-2- |
| | methoxyphenyl)amino]prop-1-yn-1-yl}-1-(2,2,2-trifluoroethyl)-1H-indol-4-amine |
| 1161. | 3-methoxy-N-methyl-4-{[3-(4-{[(1R,4R)-4-(methylamino)cyclohexyl]amino}-1- |
| | (2,2,2-trifluoroethyl)-1H-indol-2-yl)prop-2-yn-1-yl]amino}benzamide |
| 1162. | 3-methoxy-N-methyl-4-{[3-(4-{[(1R,4R)-4-aminocyclohexyl]amino}-1-(2,2,2- |
| | trifluoroethyl)-1H-indol-2-yl)prop-2-yn-1-yl]amino}benzamide |
| 1163. | 3-methoxy-N-methyl-4-{[3-(4-{[(1S,4S)-4-aminocyclohexyl]amino}-1-(2,2,2- |
| | trifluoroethyl)-1H-indol-2-yl)prop-2-yn-1-yl]amino}benzamide |
| 1164. | rel-(1R,3R)-N¹-(2-{3-[(4-methanesulfonyl-2-methoxyphenyl)amino]prop-1-yn-1- |
| | yl}-1-(2,2,2-trifluoroethyl)-1H-indol-4-yl)cyclohexane-1,3-diamine |
| 1165. | rac-(1R,3S)-N¹-(2-{3-[(4-methanesulfonyl-2-methoxyphenyl)amino]prop-1-yn-1- |
| | yl}-1-(2,2,2-trifluoroethyl)-1H-indol-4-yl)cyclohexane-1,3-diamine |
| 1166. | (1R,2S)-N¹-(2-{3-[(4-methanesulfonyl-2-methoxyphenyl)amino]prop-1-yn-1-yl}- |
| | 1-(2,2,2-trifluoroethyl)-1H-indol-4-yl)cyclohexane-1,2-diamine |
| 1167. | rac-(1R,2S)-N¹-(2-{3-[(4-methanesulfonyl-2-methoxyphenyl)amino]prop-1-yn-1- |
| | yl}-1-(2,2,2-trifluoroethyl)-1H-indol-4-yl)cyclohexane-1,2-diamine |
| 1168. | rel-(1R,3S)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexan-1-ol |
| 1169. | 3-methoxy-N-methyl-4-{[3-(4-{[(1R,4R)-4-hydroxy-4-methylcyclohexyl]amino}- |
| | 1-(2,2,2-trifluoroethyl)-1H-indol-2-yl)prop-2-yn-1-yl]amino}benzami de |
| 1170. | 3-methoxy-N-methyl-4-{[3-(4-{[(1S,4S)-4-hydroxy-4-methylcyclohexyl]amino}- |
| | 1-(2,2,2-trifluoroethyl)-1H-indol-2-yl)prop-2-yn-1-yl]amino}benzamide |
| 1171. | 4-[(3-{4-[(4-cyanocyclohexyl)amino]-1-(2,2,2-trifluoroethyl)-1H-indol-2-yl}prop- |
| | 2-yn-1-yl)amino]-3-methoxy-N-methylbenzamide |
| 1172. | 3-methoxy-N-methyl-4-{[3-(4-{[(1R,4R)-4-cyanocyclohexyl]amino}-1-(2,2,2- |
| | trifluoroethyl)-1H-indol-2-yl)prop-2-yn-1-yl]amino}benzamide |
| 1173. | 3-methoxy-N-methyl-4-{[3-(4-{[(1S,4S)-4-cyanocyclohexyl]amino}-1-(2,2,2- |
| | trifluoroethyl)-1H-indol-2-yl)prop-2-yn-1-yl]amino}benzamide |
| 1174. | 3-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexane-1-carboxylic acid |
| 1175. | 2-fluoro-N¹-(2-(3-((2-methoxy-4-(methylsulfonyl)phenyl)amino)prop-1-yn-1-yl)- |
| | 1-(2,2,2-trifluoroethyl)-1H-indol-4-yl)cyclohexane-1,4-diamine |
| 1176. | (1R,2R,4S)-2-fluoro-N¹-(2-(3-((2-methoxy-4-(methylsulfonyl)phenyl)amino)prop- |
| | 1-yn-1-yl)-1-(2,2,2-trifluoroethyl)-1H-indol-4-yl)cyclohexane-1,4-diamine |
| 1177. | 2-fluoro-N¹-(2-{3-[(4-methanesulfonyl-2-methoxyphenyl)amino]prop-1-yn-1-yl}- |
| | 1-(2,2,2-trifluoroethyl)-1H-indol-4-yl)-N⁴-methylcyclohexane-1,4-diamine |
| 1178. | (1R,2R,4S)-2-fluoro-N¹-(2-(3-((2-methoxy-4-(methylsulfonyl)phenyl)amino)prop- |
| | 1-yn-1-yl)-1-(2,2,2-trifluoroethyl)-1H-indol-4-yl)-N⁴-methylcyclohexane-1,4- |
| | diamine |
| 1179. | 2-fluoro-N¹-(2-(3-((2-methoxy-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 1180. | (1R,2R,4S)-2-fluoro-N¹-(2-(3-((2-methoxy-4-(methylsulfonyl)phenyl)amino)prop- |
| | 1-yn-1-yl)-1-(2,2,2-trifluoroethyl)-1H-indol-4-yl)-N⁴,N⁴-dimethylcyclohexane- |
| | 1,4-diamine |
| 1181. | (3S,4R)-3-fluoro-4-[(2-{3-[(4-methanesulfonyl-2-methoxyphenyl)amino]prop-1- |
| | yn-1-yl}-1-(2,2,2-trifluoroethyl)-1H-indol-4-yl)amino]cyclohexan-1-ol |
| 1182. | 3-methoxy-4-{[3-(4-{[(1R,4R)-4-[bis(2-hydroxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 1183. | 3-methoxy-4-{[3-(4-{[(1S,4S)-4-[bis(2-hydroxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 1184. | 4-((3-(4-(((1R,4R)-4-(dimethylamino)cyclohexyl)amino)-1-(2,2,2-trifluoroethyl)- |
| | 1H-indol-2-yl)prop-2-yn-1-yl)amino)-3-methoxybenzenesulfonamide |
| 1185. | 4-((3-(4-(((1S,4S)-4-(dimethylamino)cyclohexyl)amino)-1-(2,2,2-trifluoroethyl)- |
| | 1H-indol-2-yl)prop-2-yn-1-yl)amino)-3-methoxybenzenesulfonamide |
| 1186. | 3-methoxy-N-(1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1187. | 3-methoxy-N-(oxan-4-yl)-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1188. | 3-methoxy-N-(1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1189. | 3-methoxy-N-(oxan-4-yl)-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1190. | N-(3-(4-(((1S,4S)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)propionamide |
| 1191. | N-(3-(4-(((1R,4R)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)propionamide |
| 1192. | 3-methoxy-N-(5-methyl-1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1193. | 3-methoxy-N-(5-methyl-1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1194. | (1S,4S)-N⁴-(2-{3-[(4-methanesulfonyl-2-methoxyphenyl)amino]prop-1-yn-1-yl}- |
| | 1-(2,2,2-trifluoroethyl)-1H-indol-4-yl)-N¹-(2-methoxyethyl)-N¹- |
| | methylcyclohexane-1,4-diamine |
| 1195. | (1R,4R)-N⁴-(2-{3-[(4-methanesulfonyl-2-methoxyphenyl)amino]prop-1-yn-1-yl}- |
| | 1-(2,2,2-trifluoroethyl)-1H-indol-4-yl)-N¹-(2-methoxyethyl)-N¹- |
| | methylcyclohexane-1,4-diamine |
| 1196. | 3-methoxy-4-{[3-(4-{[(1S,4S)-4-[(3S,4S)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 1197. | 4-((3-(4-(((1R,4R)-4-((3R,4R)-3,4-dihydroxypyrrolidin-1-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1198. | 4-((3-(4-(((1R,4S)-4-((3S,4S)-3,4-dihydroxypyrrolidin-1-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1199. | 3-methoxy-4-{[3-(4-{[(1R,4R)-4-[bis(2-methoxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 1200. | 3-methoxy-4-{[3-(4-{[(1S,4S)-4-[bis(2-methoxyethyl)amino]cyclohexyl]amino}- |
| | 1-(2,2,2-trifluoroethyl)-1H-indol-2-yl)prop-2-yn-1-yl]amino}benzene-1- |
| | sulfonamide |
| 1201. | 4-({3-[4-({1,4-dioxaspiro[4.5]decan-8-yl}amino)-1-(2,2,2-trifluoroethyl)-1H- |
| | indol-2-yl]prop-2-yn-1-yl}amino)-3-methoxybenzene-1-sulfonamide |
| 1202. | N-{1,4-dioxaspiro[4.5]decan-8-yl}-2-{3-[(4-methanesulfonyl-2- |
| | methoxyphenyl)amino]prop-1-yn-1-yl}-1-(2,2,2-trifluoroethyl)-1H-indol-4-amine |
| 1203. | 3-methoxy-4-[(3-{4-[(4-oxocyclohexyl)amino]-1-(2,2,2-trifluoroethyl)-1H-indol- |
| | 2-yl}prop-2-yn-1-yl)amino]benzene-1-sulfonamide |
| 1204. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-one |
| 1205. | (1R,4R)-N⁴-[2-(3-{[2-methoxy-4-(morpholine-4-sulfonyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 1206. | (1S,4S)-N⁴-[2-(3-{[2-methoxy-4-(morpholine-4-sulfonyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 1207. | N,N-bis(2-hydroxyethyl)-3-methoxy-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1208. | N,N-bis(2-hydroxyethyl)-3-methoxy-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1209. | 3-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzene-1- |
| | sulfonamide |
| 1210. | 3-methoxy-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1211. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1212. | 3-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1213. | 3-methoxy-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 1214. | (1R,4R)-N⁴-{2-[3-({2-methoxy-4-[(4-methylpiperazin-1- |
| | yl)sulfonyl]phenyl}amino)prop-1-yn-1-yl]-1-(2,2,2-trifluoroethyl)-1H-indol-4-yl}- |
| | N¹,N¹-dimethylcyclohexane-1,4-diamine |
| 1215. | (1R,4R)-N⁴-(2-{3-[(4-methanesulfonyl-2-methoxyphenyl)amino]prop-1-yn-1-yl}- |
| | 1-[(oxiran-2-yl)methyl]-1H-indol-4-yl)-N¹,N¹-dimethylcyclohexane-1,4-diamine |
| 1216. | 2-[(2-{3-[(4-methanesulfonyl-2-methoxyphenyl)amino]prop-1-yn-1-yl}-4- |
| | {[(1R,4R)-4-(dimethylamino)cyclohexyl]amino}-1H-indol-1-yl)methyl]prop-2- |
| | enenitrile |
| 1217. | 5-methanesulfonyl-2-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}phenol |
| 1218. | 5-methanesulfonyl-2-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}phenol |
| 1219. | 2-(5-methanesulfonyl-2-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}phenoxy)acetonitrile |
| 1220. | 2-(5-methanesulfonyl-2-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}phenoxy)acetonitrile |
| 1221. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 1222. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 1223. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1- |
| | yl]amino}benzenesulfonyl)propanamide |
| 1224. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1- |
| | yl]amino}benzenesulfonyl)propanamide |
| 1225. | 2-(5-methanesulfonyl-2-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}phenoxy)ethan-1-ol |
| 1226. | 2-(5-methanesulfonyl-2-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}phenoxy)ethan-1-ol |
| 1227. | (1R,4R)-N⁴-[2-(3-{[4-methanesulfonyl-2-(2-methoxyethoxy)phenyl]amino}prop- |
| | 1-yn-1-yl)-1-(2,2,2-trifluoroethyl)-1H-indol-4-yl]-N¹,N¹-dimethylcyclohexane- |
| | 1,4-diamine |
| 1228. | N-(3-(4-(((1R,4R)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)isobutyramide |
| 1229. | N-(3-(4-(((1S,4S)-4-(dimethylamino)cyclohexyl)amino)-1-(2,2,2-trifluoroethyl)- |
| | 1H-indol-2-yl)prop-2-yn-1-yl)-N-(2-hydroxy-4- |
| | (methylsulfonyl)phenyl)isobutyramide |
| 1230. | (1R,4R)-N⁴-[2-(3-{[4-methanesulfonyl-2-(2,2,2- |
| | trifluoroethoxy)phenyl]amino}prop-1-yn-1-yl)-1-(2,2,2-trifluoroethyl)-1H-indol- |
| | 4-yl]-N¹,N¹-dimethylcyclohexane-1,4-diamine |
| 1231. | (1S,4S)-N⁴-[2-(3-{[4-methanesulfonyl-2-(2,2,2- |
| | trifluoroethoxy)phenyl]amino}prop-1-yn-1-yl)-1-(2,2,2-trifluoroethyl)-1H-indol- |
| | 4-yl]-N¹,N¹-dimethylcyclohexane-1,4-diamine |
| 1232. | (1S,4S)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 1233. | (1R,4R)-N⁴-(2-{3-[(2-ethoxy-4-methanesulfonylphenyl)amino]prop-1-yn-1-yl}-1- |
| | (2,2,2-trifluoroethyl)-1H-indol-4-yl)-N¹,N¹-dimethylcyclohexane-1,4-diamine |
| 1234 | (1S,4S)-N⁴-(2-{3-[(2-ethoxy-4-methanesulfonylphenyl)amino]prop-1-yn-1-yl}-1- |
| | (2,2,2-trifluoroethyl)-1H-indol-4-yl)-N¹,N¹-dimethylcyclohexane-1,4-diamine |
| 1235. | 3-hydroxy-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 1236. | 3-hydroxy-N-methyl-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzamide |
| 1237. | 3-(2-methoxyethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzamide |
| 1238. | (1R,4R)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 1239. | N-ethyl-3-methoxy-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzamide |
| 1240. | N-ethyl-3-methoxy-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 1241. | 3-(2-methoxyethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 1242. | 3-(2-fluoroethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 1243. | 3-(2-fluoroethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzamide |
| 1244. | 3-(fluoromethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 1245. | 3-(fluoromethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2- |
| | yn-1-yl]amino}benzamide |
| 1246. | (1S,4S)-N⁴-(2-{3-[4-methanesulfonyl-2-(methylamino)phenoxy]prop-1-yn-1-yl}- |
| | 1-(2,2,2-trifluoroethyl)-1H-indol-4-yl)-N¹,N¹-dimethylcyclohexane-1,4-diamine |
| 1247. | (1R,4R)-N⁴-(2-{3-[4-methanesulfonyl-2-(methylamino)phenoxy]prop-1-yn-1-yl}- |
| | 1-(2,2,2-trifluoroethyl)-1H-indol-4-yl)-N¹,N¹-dimethylcyclohexane-1,4-diamine |
| 1248. | 3-methoxy-N-methyl-4-{[3-(4-{[(1S,4S)-4-(methylamino)cyclohexyl]amino}-1- |
| | (2,2,2-trifluoroethyl)-1H-indol-2-yl)prop-2-yn-1-yl]amino}benzamide |
| 1249. | (1S,4S)-N⁴-(2-{3-[2-(dimethylamino)-4-methanesulfonylphenoxy]prop-1-yn-1- |
| | yl}-1-(2,2,2-trifluoroethyl)-1H-indol-4-yl)-N¹,N¹-dimethylcyclohexane-1,4- |
| | diamine |
| 1250. | (1R,4R)-N⁴-(2-{3-[2-(dimethylamino)-4-methanesulfonylphenoxy]prop-1-yn-1- |
| | yl}-1-(2,2,2-trifluoroethyl)-1H-indol-4-yl)-N¹,N¹-dimethylcyclohexane-1,4- |
| | diamine |
| 1251. | 3-(cyanomethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 1252. | 3-(2-cyanoethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 1253. | N-(5-methanesulfonyl-2-{[3-(4-{[(1R,4R)-4-(dimethylamino)cyclohexyl]amino}- |
| | 1-(2,2,2-trifluoroethyl)-1H-indol-2-yl)prop-2-yn-1-yl]oxy}phenyl)acetamide |
| 1254. | N-(2-((3-(4-(((1S,4S)-4-(dimethylamino)cyclohexyl)amino)-1-(2,2,2- |
| | trifluoroethyl)-1H-indol-2-yl)prop-2-yn-1-yl)oxy)-5- |
| | (methylsulfonyl)phenyl)acetamide |
| 1255. | (1R,4R)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 1256. | (1S,4S)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 1257. | (1R,4R)-N⁴-[2-(3-{[4-(ethanesulfonyl)-2-methoxyphenyl]amino}prop-1-yn-1-yl)- |
| | 1-(2,2,2-trifluoroethyl)-1H-indol-4-yl]-N¹,N¹-dimethylcyclohexane-1,4-diamine |
| 1258. | (1S,4S)-N⁴-[2-(3-{[4-(ethanesulfonyl)-2-methoxyphenyl]amino}prop-1-yn-1-yl)- |
| | 1-(2,2,2-trifluoroethyl)-1H-indol-4-yl]-N¹,N¹-dimethylcyclohexane-1,4-diamine |
| 1259. | 2-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenyl)-2- |
| | methylpropanenitrile |
| 1260. | 2-(4-((3-(4-(((1S,4S)-4-(dimethylamino)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1261. | 3-(cyanomethoxy)-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1262. | 3-(cyanomethoxy)-4-((3-(4-(((1S,4S)-4-(dimethylamino)cyclohexyl)amino)-1- |
| | (2,2,2-trifluoroethyl)-1H-indol-2-yl)prop-2-yn-1-yl)amino)benzenesulfonamide |
| 1263. | 3-(3-(4-(((1R,4R)-4-(dimethylamino)cyclohexyl)amino)-1-(2,2,2-trifluoroethyl)- |
| | 1H-indol-2-yl)prop-2-yn-1-yl)-6-(methylsulfonyl)benzo[d]oxazol-2(3H)-one |
| 1264. | 3-(3-(4-(((1S,4S)-4-(dimethylamino)cyclohexyl)amino)-1-(2,2,2-trifluoroethyl)- |
| | 1H-indol-2-yl)prop-2-yn-1-yl)-6-(methylsulfonyl)benzo[d]oxazol-2(3H)-one |
| 1265. | 3-methoxy-N,N-dimethyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzene-1-sulfonamide |
| 1266. | (1R,4R)-N¹-(2-(3-(((3S,4R)-3-methoxytetrahydro-2H-pyran-4-yl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 1267. | (1S,4S)-N¹-(2-(3-(((3S,4R)-3-methoxytetrahydro-2H-pyran-4-yl)amino)prop-1- |
| | yn-1-yl)-1-(2,2,2-trifluoroethyl)-1H-indol-4-yl)-N⁴,N⁴-dimethylcyclohexane-1,4- |
| | diamine |
| 1268. | (1R,4R)-N⁴-(2-{3-[(4-chloro-2-methoxyphenyl)amino]prop-1-yn-1-yl}-1-(2,2,2- |
| | trifluoroethyl)-1H-indol-4-yl)-N¹,N¹-dimethylcyclohexane-1,4-diamine |
| 1269. | (1S,4S)-N¹-(2-(3-((4-chloro-2-methoxyphenyl)amino)prop-1-yn-1-yl)-1-(2,2,2- |
| | trifluoroethyl)-1H-indol-4-yl)-N⁴,N⁴-dimethylcyclohexane-1,4-diamine |
| 1270. | 4-((3-(4-(((1R,4R)-4-(dimethylamino)cyclohexyl)amino)-1-(2,2,2-trifluoroethyl)- |
| | 1H-indol-2-yl)prop-2-yn-1-yl)amino)-2-fluoro-5-methoxybenzamide |
| 1271. | 4-((3-(4-(((1S,4S)-4-(dimethylamino)cyclohexyl)amino)-1-(2,2,2-trifluoroethyl)- |
| | 1H-indol-2-yl)prop-2-yn-1-yl)amino)-2-fluoro-5-methoxybenzamide |
| 1272. | (1R,4R)-N⁴-[2-(3-{[2-methoxy-4-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1273. | (1S,4S)-N¹-(2-(3-((2-methoxy-4-(trifluoromethyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 1274. | 2-fluoro-5-methoxy-N-methyl-4-{[3-(4-{[(1R,4R)-4- |
| | (dimethylamino)cyclohexyl]amino}-1-(2,2,2-trifluoroethyl)-1H-indol-2-yl)prop-2- |
| | yn-1-yl]amino}benzamide |
| 1275. | 4-((3-(4-(((1S,4S)-4-(dimethylamino)cyclohexyl)amino)-1-(2,2,2-trifluoroethyl)- |
| | 1H-indol-2-yl)prop-2-yn-1-yl)amino)-2-fluoro-5-methoxy-N-methylbenzamide |
| 1276. | (1S,4S)-N⁴-[2-(3-{[4-methanesulfonyl-2-(trifluoromethyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 1277. | (1R,4R)-N⁴-[2-(3-{[4-methanesulfonyl-2-(trifluoromethyl)phenyl]amino}prop-1- |
| | yn-1-yl)-1-(2,2,2-trifluoroethyl)-1H-indol-4-yl]-N¹,N¹-dimethylcyclohexane-1,4- |
| | diamine |
| 1278. | (1R,4R)-N⁴-(2-{3-[(4-methanesulfonyl-2-methylphenyl)amino]prop-1-yn-1-yl}-1- |
| | (2,2,2-trifluoroethyl)-1H-indol-4-yl)-N¹,N¹-dimethylcyclohexane-1,4-diamine |
| 1279. | (1S,4S)-N¹,N¹-dimethyl-N⁴-(2-(3-((2-methyl-4- |
| | (methylsulfonyl)phenyl)amino)prop-1-yn-1-yl)-1-(2,2,2-trifluoroethyl)-1H-indol- |
| | 4-yl)cyclohexane-1,4-diamine |
| 1280. | (1R,4R)-N⁴-[2-(3-{[4-methanesulfonyl-2-(trifluoromethoxy)phenyl]amino}prop- |
| | 1-yn-1-yl)-1-(2,2,2-trifluoroethyl)-1H-indol-4-yl]-N¹,N¹-dimethylcyclohexane- |
| | 1,4-diamine |
| 1281. | (1S,4S)-N¹,N¹-dimethyl-N⁴-(2-(3-((4-(methylsulfonyl)-2- |
| | (trifluoromethoxy)phenyl)amino)prop-1-yn-1-yl)-1-(2,2,2-trifluoroethyl)-1H- |
| | indol-4-yl)cyclohexane-1,4-diamine |
| 1282. | 3-methoxy-N-methyl-4-{[3-(4-{[(1R,4R)-4-(diethylamino)cyclohexyl]amino}-1- |
| | (2,2,2-trifluoroethyl)-1H-indol-2-yl)prop-2-yn-1-yl]amino}benzamide |
| 1283. | 4-((3-(4-(((1S,4S)-4-(diethylamino)cyclohexyl)amino)-1-(2,2,2-trifluoroethyl)- |
| | 1H-indol-2-yl)prop-2-yn-1-yl)amino)-3-methoxy-N-methylbenzamide |
| 1284. | 3-methoxy-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 1285. | 4-((3-(4-(((1S,4S)-4-(diethylamino)cyclohexyl)amino)-1-(2,2,2-trifluoroethyl)- |
| | 1H-indol-2-yl)prop-2-yn-1-yl)amino)-3-methoxybenzenesulfonamide |
| 1286. | 3-methoxy-N-methyl-4-{[3-(4-{[(1R,4R)-4-(diethylamino)cyclohexyl]amino}-1- |
| | (2,2,2-trifluoroethyl)-1H-indol-2-yl)prop-2-yn-1-yl]amino}benzene-1-sulfonamide |
| 1287. | 2-(5-methanesulfonyl-2-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}phenoxy)acetonitrile |
| 1288. | 2-(5-methanesulfonyl-2-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}phenoxy)acetonitrile |
| 1289. | 3-methoxy-N-methyl-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 1290. | 3-methoxy-N-methyl-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 1291. | N-ethyl-3-methoxy-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 1292. | N-ethyl-3-methoxy-4-((3-(4-(((1S,4S)-4-((2- |
| | methoxyethyl)(methyl)amino)cyclohexyl)amino)-1-(2,2,2-trifluoroethyl)-1H- |
| | indol-2-yl)prop-2-yn-1-yl)amino)benzamide |
| 1293. | (1R,4R)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴-(2-methoxyethyl)-N⁴- |
| | methylcyclohexane-1,4-diamine |
| 1294. | (1S,4S)-N¹-(2-(3-((2-(fluoromethoxy)-4-(methylsulfonyl)phenyl)amino)prop-1- |
| | yn-1-yl)-1-(2,2,2-trifluoroethyl)-1H-indol-4-yl)-N⁴-(2-methoxyethyl)-N⁴- |
| | methylcyclohexane-1,4-diamine |
| 1295. | 3-(fluoromethoxy)-N-methyl-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 1296. | 3-(fluoromethoxy)-N-methyl-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzamide |
| 1297. | 3-(fluoromethoxy)-4-((3-(4-(((1S,4S)-4-((2- |
| | methoxyethyl)(methyl)amino)cyclohexyl)amino)-1-(2,2,2-trifluoroethyl)-1H- |
| | indol-2-yl)prop-2-yn-1-yl)amino)-N-methylbenzamide |
| 1298. | 3-methoxy-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 1299. | 3-methoxy-4-((3-(4-(((1S,4S)-4-((2- |
| | methoxyethyl)(methyl)amino)cyclohexyl)amino)-1-(2,2,2-trifluoroethyl)-1H- |
| | indol-2-yl)prop-2-yn-1-yl)amino)benzenesulfonamide |
| 1300. | (1R,4R)-N¹,N¹-diethyl-N⁴-(2-{3-[(4-methanesulfonyl-2- |
| | methoxyphenyl)amino]prop-1-yn-1-yl}-1-(2,2,2-trifluoroethyl)-1H-indol-4- |
| | yl)cyclohexane-1,4-diamine |
| 1301. | (1S,4S)-N¹,N¹-diethyl-N⁴-(2-(3-((2-methoxy-4- |
| | (methylsulfonyl)phenyl)amino)prop-1-yn-1-yl)-1-(2,2,2-trifluoroethyl)-1H-indol- |
| | 4-yl)cyclohexane-1,4-diamine |
| 1302. | 3-(cyanomethoxy)-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 1303. | 3-(cyanomethoxy)-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 1304. | 3-(fluoromethoxy)-4-{[3-(4-{[(1R,4R)-4-[bis(2- |
| | methoxyethyl)amino]cyclohexyl]amino}-1-(2,2,2-trifluoroethyl)-1H-indol-2- |
| | yl)prop-2-yn-1-yl]amino}benzene-1-sulfonamide |
| 1305. | 3-(fluoromethoxy)-4-{[3-(4-{[(1S,4S)-4-[bis(2- |
| | methoxyethyl)amino]cyclohexyl]amino}-1-(2,2,2-trifluoroethyl)-1H-indol-2- |
| | yl)prop-2-yn-1-yl]amino}benzene-1-sulfonamide |
| 1306. | 3-(fluoromethoxy)-4-{[3-(4-{[(1R,4R)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 1307. | 3-(fluoromethoxy)-4-{[3-(4-{[(1S,4S)-4-[(2- |
| | methoxyethyl)(methyl)amino]cyclohexyl]amino}-1-(2,2,2-trifluoroethyl)-1H- |
| | indol-2-yl)prop-2-yn-1-yl]amino}benzene-1-sulfonamide |
| 1308. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1309. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 1310. | 3-methoxy-N-methyl-4-{[3-(4-{[(1R,4R)-4-(morpholin-4-yl)cyclohexyl]amino}- |
| | 1-(2,2,2-trifluoroethyl)-1H-indol-2-yl)prop-2-yn-1-yl]amino}benzamide |
| 1311. | 3-methoxy-N-methyl-4-{[3-(4-{[(1S,4S)-4-(morpholin-4-yl)cyclohexyl]amino}-1- |
| | (2,2,2-trifluoroethyl)-1H-indol-2-yl)prop-2-yn-1-yl]amino}benzamide |
| 1312. | 2-(2-{3-[(4-methanesulfonyl-2-methoxyphenyl)amino]prop-1-yn-1-yl}-4- |
| | {[(1R,4R)-4-(morpholin-4-yl)cyclohexyl]amino}-1H-indol-1-yl)acetonitrile |
| 1313. | 4-({3-[1-(2-fluoroethyl)-4-{[(1R,4R)-4-(morpholin-4-yl)cyclohexyl]amino}-1H- |
| | indol-2-yl]prop-2-yn-1-yl}amino)-3-methoxy-N-methylbenzamide |
| 1314. | 1-(2-fluoroethyl)-2-{3-[(4-methanesulfonyl-2-methoxyphenyl)amino]prop-1-yn-1- |
| | yl}-N-[(1R,4R)-4-(morpholin-4-yl)cyclohexyl]-1H-indol-4-amine |
| 1315. | 4-({3-[1-(cyanomethyl)-4-{[(1R,4R)-4-(morpholin-4-yl)cyclohexyl]amino}-1H- |
| | indol-2-yl]prop-2-yn-1-yl}amino)-3-methoxy-N-methylbenzamide |
| 1316. | 2-{3-[(4-methanesulfonyl-2-methoxyphenyl)amino]prop-1-yn-1-yl}-1-propyl-N- |
| | [(1R,4R)-4-(morpholin-4-yl)cyclohexyl]-1H-indol-4-amine |
| 1317. | 2-{3-[(4-methanesulfonyl-2-methoxyphenyl)amino]prop-1-yn-1-yl}-1-(2- |
| | methylpropyl)-N-[(1R,4R)-4-(morpholin-4-yl)cyclohexyl]-1H-indol-4-amine |
| 1318. | 1-(2,2-difluoroethyl)-2-{3-[(4-methanesulfonyl-2-methoxyphenyl)amino]prop-1- |
| | yn-1-yl}-N-[(1R,4R)-4-(morpholin-4-yl)cyclohexyl]-1H-indol-4-amine |
| 1319. | 3-methoxy-4-((3-(4-(((1R,4R)-4-morpholinocyclohexyl)amino)-1-(2,2,2- |
| | trifluoroethyl)-1H-indol-2-yl)prop-2-yn-1-yl)amino)benzenesulfonamide |
| 1320. | 3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzenesulfonamide |
| 1321. | N-ethyl-3-methoxy-4-{[3-(4-{[(1R,4R)-4-(morpholin-4-yl)cyclohexyl]amino}-1- |
| | (2,2,2-trifluoroethyl)-1H-indol-2-yl)prop-2-yn-1-yl]amino}benzamide |
| 1322. | N-ethyl-3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzamide |
| 1323. | 2-(5-methanesulfonyl-2-{[3-(4-{[(1R,4R)-4-(morpholin-4-yl)cyclohexyl]amino}- |
| | 1-(2,2,2-trifluoroethyl)-1H-indol-2-yl)prop-2-yn-1-yl]amino}phenoxy)acetonitrile |
| 1324. | 2-(5-methanesulfonyl-2-{[3-(4-{[(1S,4S)-4-(morpholin-4-yl)cyclohexyl]amino}- |
| | 1-(2,2,2-trifluoroethyl)-1H-indol-2-yl)prop-2-yn-1-yl]amino}phenoxy)acetonitrile |
| 1325. | 3-methoxy-N-methyl-4-{[3-(4-{[(1R,4R)-4-(morpholin-4-yl)cyclohexyl]amino}- |
| | 1-(2,2,2-trifluoroethyl)-1H-indol-2-yl)prop-2-yn-1-yl]amino}benzene-1- |
| | sulfonamide |
| 1326. | 2-{3-[(4-methanesulfonyl-2-methoxyphenyl)amino]prop-1-yn-1-yl}-N-[(1R,4R)- |
| | 4-(3-methanesulfonylazetidin-1-yl)cyclohexyl]-1-(2,2,2-trifluoroethyl)-1H-indol- |
| | 4-amine |
| 1327. | 2-(3-((2-methoxy-4-(methylsulfonyl)phenyl)amino)prop-1-yn-1-yl)-N-((1S,4S)-4- |
| | (3-(methylsulfonyl)azetidin-1-yl)cyclohexyl)-1-(2,2,2-trifluoroethyl)-1H-indol-4- |
| | amine |
| 1328. | 3-methoxy-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzene-1-sulfonamide |
| 1329. | 3-methoxy-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzenesulfonamide |
| 1330. | 3-methoxy-N-methyl-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzamide |
| 1331. | 3-methoxy-N-methyl-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzamide |
| 1332. | 2-{3-[(4-methanesulfonyl-2-methoxyphenyl)amino]prop-1-yn-1-yl}-N-[(1R,4R)- |
| | 4-{2-azaspiro[3.3]heptan-2-yl}cyclohexyl]-1-(2,2,2-trifluoroethyl)-1H-indol-4- |
| | amine |
| 1333. | N-((1S,4S)-4-(2-azaspiro[3.3]heptan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1334. | 3-methoxy-N-methyl-4-{[3-(4-{[(1R,4R)-4-{2-azaspiro[3.3]heptan-2- |
| | yl}cyclohexyl]amino}-1-(2,2,2-trifluoroethyl)-1H-indol-2-yl)prop-2-yn-1- |
| | yl]amino}benzamide |
| 1335. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxy-N- |
| | methylbenzamide |
| 1336. | 3-methoxy-4-{[3-(4-{[(1R,4R)-4-{2-azaspiro[3.3]heptan-2-yl}cyclohexyl]amino}- |
| | 1-(2,2,2-trifluoroethyl)-1H-indol-2-yl)prop-2-yn-1-yl]amino}benzene-1- |
| | sulfonamide |
| 1337. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3- |
| | methoxybenzenesulfonamide |
| 1338. | 2-(3-((2-methoxy-4-(methylsulfonyl)phenyl)amino)prop-1-yn-1-yl)-N-(2- |
| | azaspiro[3.3]heptan-6-yl)-1-(2,2,2-trifluoroethyl)-1H-indol-4-amine |
| 1339. | 4-((3-(4-((2-azaspiro[3.3]heptan-6-yl)amino)-1-(2,2,2-trifluoroethyl)-1H-indol-2- |
| | yl)prop-2-yn-1-yl)amino)-3-methoxybenzenesulfonamide |
| 1340. | 2-{3-[(4-methanesulfonyl-2-methoxyphenyl)amino]prop-1-yn-1-yl}-N-[(1R,4R)- |
| | 4-{2-oxa-6-azaspiro[3.3]heptan-6-yl}cyclohexyl]-1-(2,2,2-trifluoroethyl)-1H- |
| | indol-4-amine |
| 1341. | 2-{3-[(4-methanesulfonyl-2-methoxyphenyl)amino]prop-1-yn-1-yl}-N-[(1S,4S)-4- |
| | {2-oxa-6-azaspiro[3.3]heptan-6-yl}cyclohexyl]-1-(2,2,2-trifluoroethyl)-1H-indol- |
| | 4-amine |
| 1342. | 3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1343. | 3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1344. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 1345. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 1346. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 1347. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 1348. | N-((1R,4R)-4-(2-oxa-6-azaspiro[3.3]heptan-6-yl)cyclohexyl)-2-(3-((2-(2- |
| | fluoroethoxy)-4-(methylsulfonyl)phenyl)amino)prop-1-yn-1-yl)-1-(2,2,2- |
| | trifluoroethyl)-1H-indol-4-amine |
| 1349. | N-((1S,4S)-4-(2-oxa-6-azaspiro[3.3]heptan-6-yl)cyclohexyl)-2-(3-((2-(2- |
| | fluoroethoxy)-4-(methylsulfonyl)phenyl)amino)prop-1-yn-1-yl)-1-(2,2,2- |
| | trifluoroethyl)-1H-indol-4-amine |
| 1350. | 3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1351. | 3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1352. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 1353. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 1354. | N-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)propionamide |
| 1355. | N-(3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)propionamide |
| 1356. | N-(2-hydroxy-4-methanesulfonylphenyl)-2-methyl-N-[3-(4-{[(1R,4R)-4-{2-oxa- |
| | 6-azaspiro[3.3]heptan-6-yl}cyclohexyl]amino}-1-(2,2,2-trifluoroethyl)-1H-indol- |
| | 2-yl)prop-2-yn-1-yl]propanamide |
| 1357. | N-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)isobutyramide |
| 1358. | 2-(3-{[4-methanesulfonyl-2-(2-methoxyethoxy)phenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 1359. | 2-(3-{[4-methanesulfonyl-2-(2-methoxyethoxy)phenyl]amino}prop-1-yn-1-yl)-N- |
| | [(1S,4S)-4-{2-oxa-6-azaspiro[3.3]heptan-6-yl}cyclohexyl]-1-(2,2,2- |
| | trifluoroethyl)-1H-indol-4-amine |
| 1360. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 1361. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 1362. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 1363. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 1364. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 1365. | N-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-N-(2-hydroxy-4- |
| | (methylsulfonyl)phenyl)acetamide |
| 1366. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 1367. | 3-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1368. | 3-methoxy-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1369. | 2-(3-{[4-methanesulfonyl-2-(2,2,2-trifluoroethoxy)phenyl]amino}prop-1-yn-1-yl)- |
| | N-[(1R,4R)-4-{2-oxa-6-azaspiro[3.3]heptan-6-yl}cyclohexyl]-1-(2,2,2- |
| | trifluoroethyl)-1H-indol-4-amine |
| 1370. | 2-(3-{[4-methanesulfonyl-2-(2,2,2-trifluoroethoxy)phenyl]amino}prop-1-yn-1-yl)- |
| | N-[(1S,4S)-4-{2-oxa-6-azaspiro[3.3]heptan-6-yl}cyclohexyl]-1-(2,2,2- |
| | trifluoroethyl)-1H-indol-4-amine |
| 1371. | 2-[3-(2-amino-4-methanesulfonylphenoxy)prop-1-yn-1-yl]-N-[(1S,4S)-4-{2-oxa- |
| | 6-azaspiro[3.3]heptan-6-yl}cyclohexyl]-1-(2,2,2-trifluoroethyl)-1H-indol-4-amine |
| 1372. | 2-{3-[(2-ethoxy-4-methanesulfonylphenyl)amino]prop-1-yn-1-yl}-N-[(1R,4R)-4- |
| | {2-oxa-6-azaspiro[3.3]heptan-6-yl}cyclohexyl]-1-(2,2,2-trifluoroethyl)-1H-indol- |
| | 4-amine |
| 1373. | 2-{3-[(2-ethoxy-4-methanesulfonylphenyl)amino]prop-1-yn-1-yl}-N-[(1S,4S)-4- |
| | {2-oxa-6-azaspiro[3.3]heptan-6-yl}cyclohexyl]-1-(2,2,2-trifluoroethyl)-1H-indol- |
| | 4-amine |
| 1374. | 3-(2-methoxyethoxy)-N-methyl-4-{[3-(4-{[(1R,4R)-4-{2-oxa-6- |
| | azaspiro[3.3]heptan-6-yl}cyclohexyl]amino}-1-(2,2,2-trifluoroethyl)-1H-indol-2- |
| | yl)prop-2-yn-1-yl]amino}benzamide |
| 1375. | 3-(2-methoxyethoxy)-N-methyl-4-{[3-(4-{[(1S,4S)-4-{2-oxa-6- |
| | azaspiro[3.3]heptan-6-yl}cyclohexyl]amino}-1-(2,2,2-trifluoroethyl)-1H-indol-2- |
| | yl)prop-2-yn-1-yl]amino}benzamide |
| 1376. | N-ethyl-3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1377. | N-ethyl-3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1378. | 3-(2-fluoroethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 1379. | 3-(2-fluoroethoxy)-N-methyl-4-{[3-(4-{[(1S,4S)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 1380. | 3-(cyanomethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 1381. | 3-(cyanomethoxy)-N-methyl-4-{[3-(4-{[(1S,4S)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 1382. | 2-{3-[4-methanesulfonyl-2-(methylamino)phenoxy]prop-1-yn-1-yl}-N-[(1S,4S)-4- |
| | {2-oxa-6-azaspiro[3.3]heptan-6-yl}cyclohexyl]-1-(2,2,2-trifluoroethyl)-1H-indol- |
| | 4-amine |
| 1383. | 2-{3-[4-methanesulfonyl-2-(methylamino)phenoxy]prop-1-yn-1-yl}-N-[(1R,4R)- |
| | 4-{2-oxa-6-azaspiro[3.3]heptan-6-yl}cyclohexyl]-1-(2,2,2-trifluoroethyl)-1H- |
| | indol-4-amine |
| 1384. | 2-[3-(2-amino-4-methanesulfonylphenoxy)prop-1-yn-1-yl]-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1385. | N-((1R,4R)-4-(2-oxa-6-azaspiro[3.3]heptan-6-yl)cyclohexyl)-2-(3-(2-amino-4- |
| | (methylsulfonyl)phenoxy)prop-1-yn-1-yl)-1-(2,2,2-trifluoroethyl)-1H-indol-4- |
| | amine |
| 1386. | 3-hydroxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1387. | 3-methoxy-4-[(3-{4-[(4-{2-oxa-6-azaspiro[3.3]heptan-6-yl}cyclohexyl)amino]-1- |
| | (2,2,2-trifluoroethyl)-1H-indol-2-yl}prop-2-yn-1-yl)amino]benzonitrile |
| 1388. | 3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzonitrile |
| 1389. | 2-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1390. | 2-(4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxyphenyl)-2- |
| | methylpropanenitrile |
| 1391. | 3-(fluoromethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 1392. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-(fluoromethoxy)-N- |
| | methylbenzamide |
| 1393. | 3-(2-cyanoethoxy)-N-methyl-4-{[3-(4-{[(1R,4R)-4-{2-oxa-6-azaspiro[3.3]heptan- |
| | 6-yl}cyclohexyl]amino}-1-(2,2,2-trifluoroethyl)-1H-indol-2-yl)prop-2-yn-1- |
| | yl]amino}benzamide |
| 1394. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-(2-cyanoethoxy)-N- |
| | methylbenzamide |
| 1395. | 2-(3-{[2-(fluoromethoxy)-4-methanesulfonylphenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 1396. | N-((1S,4S)-4-(2-oxa-6-azaspiro[3.3]heptan-6-yl)cyclohexyl)-2-(3-((2- |
| | (fluoromethoxy)-4-(methylsulfonyl)phenyl)amino)prop-1-yn-1-yl)-1-(2,2,2- |
| | trifluoroethyl)-1H-indol-4-amine |
| 1397. | 3-(cyanomethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1398. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3- |
| | (cyanomethoxy)benzenesulfonamide |
| 1399. | 3-(fluoromethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1400. | 3-(fluoromethoxy)-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1401. | 4-((3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 1402. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 1403. | N-((1R,4R)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 1404. | N-((1S,4S)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 1405. | 4-((3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N,N- |
| | dimethylbenzenesulfonamide |
| 1406. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxy-N,N- |
| | dimethylbenzenesulfonamide |
| 1407. | N-((1R,4R)-4-(2-oxa-6-azaspiro[3.3]heptan-6-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 1408. | N-((1S,4S)-4-(2-oxa-6-azaspiro[3.3]heptan-6-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 1409. | N-((1R,4R)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)- |
| | 1H-indol-4-amine |
| 1410. | N-((1S,4S)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)- |
| | 1H-indol-4-amine |
| 1411. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1412. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-2-fluoro-5- |
| | methoxybenzamide |
| 1413. | 2-fluoro-5-methoxy-N-methyl-4-{[3-(4-{[(1R,4R)-4-{2-oxa-6- |
| | azaspiro[3.3]heptan-6-yl}cyclohexyl]amino}-1-(2,2,2-trifluoroethyl)-1H-indol-2- |
| | yl)prop-2-yn-1-yl]amino}benzamide |
| 1414. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-2-fluoro-5-methoxy- |
| | N-methylbenzamide |
| 1415. | 2-(3-{[2-methoxy-4-(trifluoromethyl)phenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H- |
| | indol-4-amine |
| 1416. | N-((1S,4S)-4-(2-oxa-6-azaspiro[3.3]heptan-6-yl)cyclohexyl)-2-(3-((2-methoxy-4- |
| | (trifluoromethyl)phenyl)amino)prop-1-yn-1-yl)-1-(2,2,2-trifluoroethyl)-1H-indol- |
| | 4-amine |
| 1417. | 4-((3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxybenzoic |
| | acid |
| 1418. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-3-methoxybenzoic |
| | acid |
| 1419. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzoic acid |
| 1420. | 4-((3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)amino)-2-fluoro-5- |
| | methoxybenzoic acid |
| 1421. | 2-{3-[(4-methanesulfonyl-2-methylphenyl)amino]prop-1-yn-1-yl}-N-[(1R,4R)-4- |
| | {2-oxa-6-azaspiro[3.3]heptan-6-yl}cyclohexyl]-1-(2,2,2-trifluoroethyl)-1H-indol- |
| | 4-amine |
| 1422. | N-((1S,4S)-4-(2-oxa-6-azaspiro[3.3]heptan-6-yl)cyclohexyl)-2-(3-((2-methyl-4- |
| | (methylsulfonyl)phenyl)amino)prop-1-yn-1-yl)-1-(2,2,2-trifluoroethyl)-1H-indol- |
| | 4-amine |
| 1423. | 2-(3-{[4-methanesulfonyl-2-(trifluoromethoxy)phenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 1424. | N-((1S,4S)-4-(2-oxa-6-azaspiro[3.3]heptan-6-yl)cyclohexyl)-2-(3-((4- |
| | (methylsulfonyl)-2-(trifluoromethoxy)phenyl)amino)prop-1-yn-1-yl)-1-(2,2,2- |
| | trifluoroethyl)-1H-indol-4-amine |
| 1425. | 2-(3-{[4-methanesulfonyl-2-(trifluoromethyl)phenyl]amino}prop-1-yn-1-yl)-N- |
| | [(1R,4R)-4-{2-oxa-6-azaspiro[3.3]heptan-6-yl}cyclohexyl]-1-(2,2,2- |
| | trifluoroethyl)-1H-indol-4-amine |
| 1426. | N-((1S,4S)-4-(2-oxa-6-azaspiro[3.3]heptan-6-yl)cyclohexyl)-2-(3-((4- |
| | (methylsulfonyl)-2-(trifluoromethyl)phenyl)amino)prop-1-yn-1-yl)-1-(2,2,2- |
| | trifluoroethyl)-1H-indol-4-amine |
| 1427. | 2-{3-[(2-chloro-4-methanesulfonylphenyl)amino]prop-1-yn-1-yl}-N-[(1R,4R)-4- |
| | {2-oxa-6-azaspiro[3.3]heptan-6-yl}cyclohexyl]-1-(2,2,2-trifluoroethyl)-1H-indol- |
| | 4-amine |
| 1428. | N-((1S,4S)-4-(2-oxa-6-azaspiro[3.3]heptan-6-yl)cyclohexyl)-2-(3-((2-chloro-4- |
| | (methylsulfonyl)phenyl)amino)prop-1-yn-1-yl)-1-(2,2,2-trifluoroethyl)-1H-indol- |
| | 4-amine |
| 1429. | 3-(3-(4-(((1R,4R)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-6- |
| | (methylsulfonyl)benzo[d]oxazol-2(3H)-one |
| 1430. | 3-(3-(4-(((1S,4S)-4-(2-oxa-6-azaspiro[3.3]heptan-6-yl)cyclohexyl)amino)-1- |
| | (2,2,2-trifluoroethyl)-1H-indol-2-yl)prop-2-yn-1-yl)-6- |
| | (methylsulfonyl)benzo[d]oxazol-2(3H)-one |
| 1431. | 2-{3-[(4-methanesulfonyl-2-methoxyphenyl)amino]prop-1-yn-1-yl}-N-[(1S,4S)-4- |
| | {7-oxa-2-azaspiro[3.5]nonan-2-yl}cyclohexyl]-1-(2,2,2-trifluoroethyl)-1H-indol- |
| | 4-amine |
| 1432. | 2-{3-[(4-methanesulfonyl-2-methoxyphenyl)amino]prop-1-yn-1-yl}-N-[(1R,4R)- |
| | 4-{7-oxa-2-azaspiro[3.5]nonan-2-yl}cyclohexyl]-1-(2,2,2-trifluoroethyl)-1H- |
| | indol-4-amine |
| 1433. | 3-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1434. | 3-methoxy-N-methyl-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1435. | 3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1436. | 3-methoxy-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1437. | 3-(cyanomethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1438. | 3-(cyanomethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1439. | 2-(3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1440. | 2-(4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxyphenyl)-2- |
| | methylpropanenitrile |
| 1441. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1442. | 2-(5-methanesulfonyl-2-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1443. | N-((4-((3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)acetamide |
| 1444. | N-((4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)acetamide |
| 1445. | N-((4-((3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)-2-aminoacetamide |
| 1446. | N-((4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxyphenyl)sulfonyl)-2-aminoacetamide |
| 1447. | methyl 2-(5-methanesulfonyl-2-{[3-(4-{[(1R,4R)-4-{7-oxa-2-azaspiro[3.5]nonan- |
| | 2-yl}cyclohexyl]amino}-1-(2,2,2-trifluoroethyl)-1H-indol-2-yl)prop-2-yn-1- |
| | yl]amino}phenoxy)acetate |
| 1448. | methyl 2-(2-((3-(4-(((1S,4S)-4-(7-oxa-2-azaspiro[3.5]nonan-2- |
| | yl)cyclohexyl)amino)-1-(2,2,2-trifluoroethyl)-1H-indol-2-yl)prop-2-yn-1- |
| | yl)amino)-5-(methylsulfonyl)phenoxy)acetate |
| 1449. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetic acid |
| 1450. | 2-(2-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetic acid |
| 1451. | 2-{3-[(4-methanesulfonyl-2-methoxyphenyl)amino]prop-1-yn-1-yl}-N-[(1R,4R)- |
| | 4-{2-oxa-7-azaspiro[3.5]nonan-7-yl}cyclohexyl]-1-(2,2,2-trifluoroethyl)-1H- |
| | indol-4-amine |
| 1452. | 2-(3-{[4-(ethanesulfonyl)-2-methoxyphenyl]amino}prop-1-yn-1-yl)-N-[(1R,4R)- |
| | 4-{7-oxa-2-azaspiro[3.5]nonan-2-yl}cyclohexyl]-1-(2,2,2-trifluoroethyl)-1H- |
| | indol-4-amine |
| 1453. | 2-(3-{[4-(ethanesulfonyl)-2-methoxyphenyl]amino}prop-1-yn-1-yl)-N-[(1S,4S)-4- |
| | {7-oxa-2-azaspiro[3.5]nonan-2-yl}cyclohexyl]-1-(2,2,2-trifluoroethyl)-1H-indol- |
| | 4-amine |
| 1454. | 3-(fluoromethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1455. | 3-(fluoromethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1456. | 3-methoxy-N-methyl-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1457. | 4-((3-(4-(((1S,4S)-4-(6-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxy-N- |
| | methylbenzamide |
| 1458. | 3-methoxy-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1459. | 4-((3-(4-(((1S,4S)-4-(6-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1460. | 2-{3-[(4-methanesulfonyl-2-methoxyphenyl)amino]prop-1-yn-1-yl}-N-[(1R,4R)- |
| | 4-{6-oxa-2-azaspiro[3.5]nonan-2-yl}cyclohexyl]-1-(2,2,2-trifluoroethyl)-1H- |
| | indol-4-amine |
| 1461. | N-((1S,4S)-4-(6-oxa-2-azaspiro[3.5]nonan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1462. | N-((1R,4R)-4-(7-oxa-2-azaspiro[3.5]nonan-2-yl)cyclohexyl)-2-(3-((2- |
| | (fluoromethoxy)-4-(methylsulfonyl)phenyl)amino)prop-1-yn-1-yl)-1-(2,2,2- |
| | trifluoroethyl)-1H-indol-4-amine |
| 1463. | N-((1S,4S)-4-(7-oxa-2-azaspiro[3.5]nonan-2-yl)cyclohexyl)-2-(3-((2- |
| | (fluoromethoxy)-4-(methylsulfonyl)phenyl)amino)prop-1-yn-1-yl)-1-(2,2,2- |
| | trifluoroethyl)-1H-indol-4-amine |
| 1464. | 4-(3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)-7-(methylsulfonyl)-2H- |
| | benzo[b][1,4]oxazin-3(4H)-one |
| 1465. | 4-((3-(4-(((1R,4R)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 1466. | 4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxy-N- |
| | methylbenzenesulfonamide |
| 1467. | N-((1R,4R)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1468. | N-((1S,4S)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1469. | 3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1470. | 4-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxybenzamide |
| 1471. | 3-methoxy-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1472. | 4-((3-(4-(((1S,4S)-4-(6-oxa-2-azaspiro[3.5]nonan-2-yl)cyclohexyl)amino)-1- |
| | (2,2,2-trifluoroethyl)-1H-indol-2-yl)prop-2-yn-1-yl)amino)-3-methoxybenzamide |
| 1473. | 2-fluoro-5-methoxy-N-methyl-4-{[3-(4-{[(1R,4R)-4-{7-oxa-2- |
| | azaspiro[3.5]nonan-2-yl}cyclohexyl]amino}-1-(2,2,2-trifluoroethyl)-1H-indol-2- |
| | yl)prop-2-yn-1-yl]amino}benzamide |
| 1474. | 2-(3-((2-methoxy-4-(methylsulfonyl)phenyl)amino)prop-1-yn-1-yl)-N-((1R,4R)-4- |
| | (3-methoxypyrrolidin-1-yl)cyclohexyl)-1-(2,2,2-trifluoroethyl)-1H-indol-4-amine |
| 1475. | 2-(3-((2-methoxy-4-(methylsulfonyl)phenyl)amino)prop-1-yn-1-yl)-N-((1S,4S)-4- |
| | (3-methoxypyrrolidin-1-yl)cyclohexyl)-1-(2,2,2-trifluoroethyl)-1H-indol-4-amine |
| 1476. | 3-methoxy-4-((3-(4-(((1R,4R)-4-(3-methoxypyrrolidin-1-yl)cyclohexyl)amino)-1- |
| | (2,2,2-trifluoroethyl)-1H-indol-2-yl)prop-2-yn-1-yl)amino)benzenesulfonamide |
| 1477. | 3-methoxy-4-((3-(4-(((1S,4S)-4-(3-methoxypyrrolidin-1-yl)cyclohexyl)amino)-1- |
| | (2,2,2-trifluoroethyl)-1H-indol-2-yl)prop-2-yn-1-yl)amino)benzenesulfonamide |
| 1478. | 2-(3-{[2-(fluoromethoxy)-4-methanesulfonylphenyl]amino}prop-1-yn-1-yl)-N- |
| | [(1R,4R)-4-(morpholin-4-yl)cyclohexyl]-1-(2,2,2-trifluoroethyl)-1H-indol-4- |
| | amine |
| 1479. | 2-(3-{[2-(fluoromethoxy)-4-methanesulfonylphenyl]amino}prop-1-yn-1-yl)-N- |
| | [(1S,4S)-4-(morpholin-4-yl)cyclohexyl]-1-(2,2,2-trifluoroethyl)-1H-indol-4-amine |
| 1480. | 3-(cyanomethoxy)-4-{[3-(4-{[(1R,4R)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1481. | 3-(cyanomethoxy)-4-{[3-(4-{[(1S,4S)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1482. | 3-(fluoromethoxy)-4-{[3-(4-{[(1R,4R)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1483. | 3-(fluoromethoxy)-4-{[3-(4-{[(1S,4S)-4-(morpholin-4-yl)cyclohexyl]amino}-1- |
| | (2,2,2-trifluoroethyl)-1H-indol-2-yl)prop-2-yn-1-yl]amino}benzene-1-sulfonamide |
| 1484. | 2-(3-((2-ethoxy-4-(methylsulfonyl)phenyl)amino)prop-1-yn-1-yl)-N-((1R,4R)-4- |
| | morpholinocyclohexyl)-1-(2,2,2-trifluoroethyl)-1H-indol-4-amine |
| 1485. | 2-(3-((2-ethoxy-4-(methylsulfonyl)phenyl)amino)prop-1-yn-1-yl)-N-((1S,4S)-4- |
| | morpholinocyclohexyl)-1-(2,2,2-trifluoroethyl)-1H-indol-4-amine |
| 1486. | 2-{3-[(4-methanesulfonyl-2-methoxyphenyl)amino]prop-1-yn-1-yl}-N-[(1R,4R)- |
| | 4-(3-methoxypiperidin-1-yl)cyclohexyl]-1-(2,2,2-trifluoroethyl)-1H-indol-4-amine |
| 1487. | 2-(3-((2-methoxy-4-(methylsulfonyl)phenyl)amino)prop-1-yn-1-yl)-N-((1S,4S)-4- |
| | (3-methoxypiperidin-1-yl)cyclohexyl)-1-(2,2,2-trifluoroethyl)-1H-indol-4-amine |
| 1488. | 2-{3-[(4-methanesulfonyl-2-methoxyphenyl)amino]prop-1-yn-1-yl}-N-[(1R,4R)- |
| | 4-(4-methoxypiperidin-1-yl)cyclohexyl]-1-(2,2,2-trifluoroethyl)-1H-indol-4-amine |
| 1489. | 2-(3-((2-methoxy-4-(methylsulfonyl)phenyl)amino)prop-1-yn-1-yl)-N-((1S,4S)-4- |
| | (4-methoxypiperidin-1-yl)cyclohexyl)-1-(2,2,2-trifluoroethyl)-1H-indol-4-amine |
| 1490. | 3-methoxy-N-methyl-4-{[3-(4-{[(1R,4R)-4-(4-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1491. | 3-methoxy-4-((3-(4-(((1S,4S)-4-(4-methoxypiperidin-1-yl)cyclohexyl)amino)-1- |
| | (2,2,2-trifluoroethyl)-1H-indol-2-yl)prop-2-yn-1-yl)amino)-N-methylbenzamide |
| 1492. | 3-methoxy-4-{[3-(4-{[(1R,4R)-4-(4-methoxypiperidin-1-yl)cyclohexyl]amino}-1- |
| | (2,2,2-trifluoroethyl)-1H-indol-2-yl)prop-2-yn-1-yl]amino}benzene-1-sulfonamide |
| 1493. | 3-methoxy-4-((3-(4-(((1S,4S)-4-(4-methoxypiperidin-1-yl)cyclohexyl)amino)-1- |
| | (2,2,2-trifluoroethyl)-1H-indol-2-yl)prop-2-yn-1-yl)amino)benzenesulfonamide |
| 1494. | 3-methoxy-N-methyl-4-{[3-(4-{[(1R,4R)-4-(3-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1495. | 3-methoxy-4-((3-(4-(((1S,4S)-4-(3-methoxypiperidin-1-yl)cyclohexyl)amino)-1- |
| | (2,2,2-trifluoroethyl)-1H-indol-2-yl)prop-2-yn-1-yl)amino)-N-methylbenzamide |
| 1496. | 3-methoxy-4-{[3-(4-{[(1R,4R)-4-(3-methoxypiperidin-1-yl)cyclohexyl]amino}-1- |
| | (2,2,2-trifluoroethyl)-1H-indol-2-yl)prop-2-yn-1-yl]amino}benzene-1-sulfonamide |
| 1497. | 3-methoxy-4-((3-(4-(((1S,4S)-4-(3-methoxypiperidin-1-yl)cyclohexyl)amino)-1- |
| | (2,2,2-trifluoroethyl)-1H-indol-2-yl)prop-2-yn-1-yl)amino)benzenesulfonamide |
| 1498. | 3-methoxy-4-((3-(4-(((1R,4R)-4-morpholinocyclohexyl)amino)-1-(2,2,2- |
| | trifluoroethyl)-1H-indol-2-yl)prop-2-yn-1-yl)amino)benzenesulfonamide |
| 1499. | 3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzenesulfonamide |
| 1500. | 4-[(1R,4R)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-1λ⁶-thiomorpholine- |
| | 1,1-dione |
| 1501. | 4-((1S,4S)-4-((2-(3-((2-methoxy-4-(methylsulfonyl)phenyl)amino)prop-1-yn-1- |
| | yl)-1-(2,2,2-trifluoroethyl)-1H-indol-4-yl)amino)cyclohexyl)thiomorpholine 1,1- |
| | dioxide |
| 1502. | 4-[(1R,4R)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-1λ⁴-thiomorpholin- |
| | 1-one |
| 1503. | 4-[(1S,4S)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-1λ⁴-thiomorpholin- |
| | 1-one |
| 1504. | 4-((3-(4-(((1R,4R)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N- |
| | methylbenzamide |
| 1505. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N- |
| | methylbenzamide |
| 1506. | 4-((1S,4S)-4-((2-(3-((2-methoxy-4-(methylsulfonyl)phenyl)amino)prop-1-yn-1- |
| | yl)-1-(2,2,2-trifluoroethyl)-1H-indol-4-yl)amino)cyclohexyl)thiomorpholine 1- |
| | oxide |
| 1507. | 3-methoxy-N-methyl-4-{[3-(4-{[(1R,4R)-4-(1-oxo-1λ⁴-thiomorpholin-4- |
| | yl)cyclohexyl]amino}-1-(2,2,2-trifluoroethyl)-1H-indol-2-yl)prop-2-yn-1- |
| | yl]amino}benzamide |
| 1508. | 3-methoxy-4-{[3-(4-{[(1R,4R)-4-(1-oxo-1λ⁴-thiomorpholin-4- |
| | yl)cyclohexyl]amino}-1-(2,2,2-trifluoroethyl)-1H-indol-2-yl)prop-2-yn-1- |
| | yl]amino}benzene-1-sulfonamide |
| 1509. | 3-methoxy-4-((3-(4-(((1S,4S)-4-(1-oxidothiomorpholino)cyclohexyl)amino)-1- |
| | (2,2,2-trifluoroethyl)-1H-indol-2-yl)prop-2-yn-1-yl)amino)benzenesulfonamide |
| 1510. | 3-methoxy-4-{[3-(4-{[(1R,4R)-4-(1,1-dioxo-1λ⁶-thiomorpholin-4- |
| | yl)cyclohexyl]amino}-1-(2,2,2-trifluoroethyl)-1H-indol-2-yl)prop-2-yn-1- |
| | yl]amino}benzene-1-sulfonamide |
| 1511. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3- |
| | methoxybenzenesulfonamide |
| 1512. | 2-{3-[(4-methanesulfonyl-2-methoxyphenyl)amino]prop-1-yn-1-yl}-N-[(1S,4S)-4- |
| | {2-oxa-7-azaspiro[3.5]nonan-7-yl}cyclohexyl]-1-(2,2,2-trifluoroethyl)-1H-indol- |
| | 4-amine |
| 1513. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1514. | 3-methoxy-N-methyl-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1515. | 3-methoxy-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1516. | 4-((3-(4-(((1S,4S)-4-(2-oxa-7-azaspiro[3.5]nonan-7-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1517. | 3-(cyanomethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1518. | 3-(cyanomethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1519. | 3-(fluoromethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1520. | 3-(fluoromethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1521. | 2-{3-[(4-methanesulfonyl-2-methoxyphenyl)amino]prop-1-yn-1-yl}-N-[(1R,4R)- |
| | 4-{1-oxa-7-azaspiro[3.5]nonan-7-yl}cyclohexyl]-1-(2,2,2-trifluoroethyl)-1H- |
| | indol-4-amine |
| 1522. | N-((1S,4S)-4-(1-oxa-7-azaspiro[3.5]nonan-7-yl)cyclohexyl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1523. | 3-methoxy-N-methyl-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1524. | 4-((3-(4-(((1S,4S)-4-(1-oxa-7-azaspiro[3.5]nonan-7-yl)cyclohexyl)amino)-1- |
| | (2,2,2-trifluoroethyl)-1H-indol-2-yl)prop-2-yn-1-yl)amino)-3-methoxy-N- |
| | methylbenzamide |
| 1525. | 3-methoxy-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1526. | 4-((3-(4-(((1S,4S)-4-(1-oxa-7-azaspiro[3.5]nonan-7-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1527. | 2-(5-methanesulfonyl-2-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1528. | 2-(5-methanesulfonyl-2-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1529. | 2-(3-{[2-(fluoromethoxy)-4-methanesulfonylphenyl]amino}prop-1-yn-1-yl)-N- |
| | [(1R,4R)-4-{2-oxa-7-azaspiro[3.5]nonan-7-yl}cyclohexyl]-1-(2,2,2- |
| | trifluoroethyl)-1H-indol-4-amine |
| 1530. | 2-(3-{[2-(fluoromethoxy)-4-methanesulfonylphenyl]amino}prop-1-yn-1-yl)-N- |
| | [(1S,4S)-4-{2-oxa-7-azaspiro[3.5]nonan-7-yl}cyclohexyl]-1-(2,2,2-trifluoroethyl)- |
| | 1H-indol-4-amine |
| 1531. | N-((1R,4R)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1532. | N-((1S,4S)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol- |
| | 4-amine |
| 1533. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1534. | N-((1R,4R)-4-(2-oxa-7-azaspiro[3.5]nonan-7-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 1535. | N-((1S,4S)-4-(2-oxa-7-azaspiro[3.5]nonan-7-yl)cyclohexyl)-2-(3-((4- |
| | (ethylsulfonyl)-2-methoxyphenyl)amino)prop-1-yn-1-yl)-1-(2,2,2-trifluoroethyl)- |
| | 1H-indol-4-amine |
| 1536. | 2-{3-[(4-methanesulfonyl-2-methoxyphenyl)amino]prop-1-yn-1-yl}-N-[(1R,4R)- |
| | 4-{6-oxa-3-azabicyclo[3.1.1]heptan-3-yl}cyclohexyl]-1-(2,2,2-trifluoroethyl)-1H- |
| | indol-4-amine |
| 1537. | 2-{3-[(4-methanesulfonyl-2-methoxyphenyl)amino]prop-1-yn-1-yl}-N-[(1S,4S)-4- |
| | {6-oxa-3-azabicyclo[3.1.1]heptan-3-yl}cyclohexyl]-1-(2,2,2-trifluoroethyl)-1H- |
| | indol-4-amine |
| 1538. | 3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1539. | 3-methoxy-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1540. | 3-methoxy-N-methyl-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1541. | 3-methoxy-N-methyl-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1542. | 2-{3-[(2-ethoxy-4-methanesulfonylphenyl)amino]prop-1-yn-1-yl}-N-[(1R,4R)-4- |
| | {6-oxa-3-azabicyclo[3.1.1]heptan-3-yl}cyclohexyl]-1-(2,2,2-trifluoroethyl)-1H- |
| | indol-4-amine |
| 1543. | 2-{3-[(2-ethoxy-4-methanesulfonylphenyl)amino]prop-1-yn-1-yl}-N-[(1S,4S)-4- |
| | {6-oxa-3-azabicyclo[3.1.1]heptan-3-yl}cyclohexyl]-1-(2,2,2-trifluoroethyl)-1H- |
| | indol-4-amine |
| 1544. | N-ethyl-3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1545. | 2-(5-methanesulfonyl-2-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}phenoxy)acetonitrile |
| 1546. | 2-(2-((3-(4-(((1S,4S)-4-(6-oxa-3-azabicyclo[3.1.1]heptan-3-yl)cyclohexyl)amino)- |
| | 1-(2,2,2-trifluoroethyl)-1H-indol-2-yl)prop-2-yn-1-yl)amino)-5- |
| | (methylsulfonyl)phenoxy)acetonitrile |
| 1547. | 3-methoxy-N-methyl-4-{[3-(4-{[(1R,4R)-4-{2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzamide |
| 1548. | 2-{3-[(4-methanesulfonyl-2-methoxyphenyl)amino]prop-1-yn-1-yl}-N-[(1R,4R)- |
| | 4-{2-oxa-8-azaspiro[4.5]decan-8-yl}cyclohexyl]-1-(2,2,2-trifluoroethyl)-1H- |
| | indol-4-amine |
| 1549. | 3-methoxy-4-{[3-(4-{[(1R,4R)-4-{2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzene-1-sulfonamide |
| 1550. | 4-((3-(4-(((1R,4R)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxybenzenesulfonamide |
| 1551. | 4-((3-(4-(((1S,4S)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxybenzenesulfonamide |
| 1552. | 2-{3-[(4-methanesulfonyl-2-methoxyphenyl)amino]prop-1-yn-1-yl}-N-[(1R,4R)- |
| | 4-{3-oxa-9-azaspiro[5.5]undecan-9-yl}cyclohexyl]-1-(2,2,2-trifluoroethyl)-1H- |
| | indol-4-amine |
| 1553. | N-((1S,4S)-4-(3-oxa-9-azaspiro[5.5]undecan-9-yl)cyclohexyl)-2-(3-((2-methoxy- |
| | 4-(methylsulfonyl)phenyl)amino)prop-1-yn-1-yl)-1-(2,2,2-trifluoroethyl)-1H- |
| | indol-4-amine |
| 1554. | 3-methoxy-N-methyl-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzamide |
| 1555. | 4-((3-(4-(((1S,4S)-4-(3-oxa-9-azaspiro[5.5]undecan-9-yl)cyclohexyl)amino)-1- |
| | (2,2,2-trifluoroethyl)-1H-indol-2-yl)prop-2-yn-1-yl)amino)-3-methoxy-N- |
| | methylbenzamide |
| 1556. | 3-methoxy-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzene-1-sulfonamide |
| 1557. | 4-((3-(4-(((1S,4S)-4-(3-oxa-9-azaspiro[5.5]undecan-9-yl)cyclohexyl)amino)-1- |
| | (2,2,2-trifluoroethyl)-1H-indol-2-yl)prop-2-yn-1-yl)amino)-3- |
| | methoxybenzenesulfonamide |
| 1558. | 3-methoxy-N-methyl-4-{[3-(4-{[(1R,4R)-4-(pyrrolidin-1-yl)cyclohexyl]amino}-1- |
| | (2,2,2-trifluoroethyl)-1H-indol-2-yl)prop-2-yn-1-yl]amino}benzamide |
| 1559. | 3-methoxy-N-methyl-4-((3-(4-(((1S,4S)-4-(pyrrolidin-1-yl)cyclohexyl)amino)-1- |
| | (2,2,2-trifluoroethyl)-1H-indol-2-yl)prop-2-yn-1-yl)amino)benzamide |
| 1560. | 3-methoxy-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1561. | 3-methoxy-4-((3-(4-(((1S,4S)-4-(pyrrolidin-1-yl)cyclohexyl)amino)-1-(2,2,2- |
| | trifluoroethyl)-1H-indol-2-yl)prop-2-yn-1-yl)amino)benzenesulfonamide |
| 1562. | 2-{3-[(4-methanesulfonyl-2-methoxyphenyl)amino]prop-1-yn-1-yl}-N-[(1R,4R)- |
| | 4-(pyrrolidin-1-yl)cyclohexyl]-1-(2,2,2-trifluoroethyl)-1H-indol-4-amine |
| 1563. | 2-(3-((2-methoxy-4-(methylsulfonyl)phenyl)amino)prop-1-yn-1-yl)-N-((1S,4S)-4- |
| | (pyrrolidin-1-yl)cyclohexyl)-1-(2,2,2-trifluoroethyl)-1H-indol-4-amine |
| 1564. | 2-{3-[(4-methanesulfonyl-2-methoxyphenyl)amino]prop-1-yn-1-yl}-N-[(1R,4R)- |
| | 4-(3-methoxypyrrolidin-1-yl)cyclohexyl]-1-(2,2,2-trifluoroethyl)-1H-indol-4- |
| | amine |
| 1565. | 3-methoxy-4-{[3-(4-{[(1R,4R)-4-(3-methoxypyrrolidin-1-yl)cyclohexyl]amino}- |
| | 1-(2,2,2-trifluoroethyl)-1H-indol-2-yl)prop-2-yn-1-yl]amino}benzene-1- |
| | sulfonamide |
| 1566. | 3-methoxy-N-methyl-4-{[3-(4-{[(1R,4R)-4-(3-methoxypyrrolidin-1- |
| | yl)cyclohexyl]amino}-1-(2,2,2-trifluoroethyl)-1H-indol-2-yl)prop-2-yn-1- |
| | yl]amino}benzamide |
| 1567. | 3-methoxy-4-((3-(4-(((1S,4S)-4-(3-methoxypyrrolidin-1-yl)cyclohexyl)amino)-1- |
| | (2,2,2-trifluoroethyl)-1H-indol-2-yl)prop-2-yn-1-yl)amino)-N-methylbenzamide |
| 1568. | 3-methoxy-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzene-1-sulfonamide |
| 1569. | 3-methoxy-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzenesulfonamide |
| 1570. | 2-{3-[(4-methanesulfonyl-2-methoxyphenyl)amino]prop-1-yn-1-yl}-N-[(1R,4R)- |
| | 4-{hexahydro-1H-furo[3,4-c]pyrrol-5-yl}cyclohexyl]-1-(2,2,2-trifluoroethyl)-1H- |
| | indol-4-amine |
| 1571. | 2-(3-((2-methoxy-4-(methylsulfonyl)phenyl)amino)prop-1-yn-1-yl)-N-((1R,4R)-4- |
| | (tetrahydro-1H-furo[3,4-c]pyrrol-5(3H)-yl)cyclohexyl)-1-(2,2,2-trifluoroethyl)- |
| | 1H-indol-4-amine |
| 1572. | 3-methoxy-N-methyl-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzamide |
| 1573. | 3-methoxy-N-methyl-4-((3-(4-(((1S,4S)-4-(tetrahydro-1H-furo[3,4-c]pyrrol- |
| | 5(3H)-yl)cyclohexyl)amino)-1-(2,2,2-trifluoroethyl)-1H-indol-2-yl)prop-2-yn-1- |
| | yl)amino)benzamide |
| 1574. | 2-{3-[(4-methanesulfonyl-2-methoxyphenyl)amino]prop-1-yn-1-yl}-N-[(1R,4R)- |
| | 4-[4-(trifluoromethyl)piperidin-1-yl]cyclohexyl]-1-(2,2,2-trifluoroethyl)-1H-indol- |
| | 4-amine |
| 1575. | 2-(3-((2-methoxy-4-(methylsulfonyl)phenyl)amino)prop-1-yn-1-yl)-1-(2,2,2- |
| | trifluoroethyl)-N-((1S,4S)-4-(4-(trifluoromethyl)piperidin-1-yl)cyclohexyl)-1H- |
| | indol-4-amine |
| 1576. | 3-methoxy-N-methyl-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzamide |
| 1577. | 3-methoxy-N-methyl-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzamide |
| 1578. | 3-methoxy-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 1579. | 3-methoxy-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzenesulfonamide |
| 1580. | 2-{3-[(4-methanesulfonyl-2-methoxyphenyl)amino]prop-1-yn-1-yl}-N-[(1R,4R)- |
| | 4-(4-methanesulfonylpiperidin-1-yl)cyclohexyl]-1-(2,2,2-trifluoroethyl)-1H-indol- |
| | 4-amine |
| 1581. | 2-(3-((2-methoxy-4-(methylsulfonyl)phenyl)amino)prop-1-yn-1-yl)-N-((1S,4S)-4- |
| | (4-(methylsulfonyl)piperidin-1-yl)cyclohexyl)-1-(2,2,2-trifluoroethyl)-1H-indol-4- |
| | amine |
| 1582. | 3-methoxy-N-methyl-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzamide |
| 1583. | 3-methoxy-N-methyl-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzamide |
| 1584. | 3-methoxy-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzene-1-sulfonamide |
| 1585. | 3-methoxy-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzenesulfonamide |
| 1586. | 1-[(1S,3R)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]-4,5-dihydro-1H- |
| | 1,2,3,4-tetrazol-5-one |
| 1587. | 2-((2-(3-((2-methoxy-4-(methylsulfonyl)phenyl)amino)prop-1-yn-1-yl)-4- |
| | (((1R,4R)-4-morpholinocyclohexyl)amino)-1H-indol-1-yl)methyl)acrylonitrile |
| 1588. | N-((1R,4R)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1589. | N-((1S,4S)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1590. | 4-((3-(4-(((1R,4R)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxy-N-methylbenzamide |
| 1591. | 4-((3-(4-(((1S,4S)-4-(2-oxa-8-azaspiro[4.5]decan-8-yl)cyclohexyl)amino)-1- |
| | (2,2,2-trifluoroethyl)-1H-benzo[d]imidazol-2-yl)prop-2-yn-1-yl)amino)-3- |
| | methoxy-N-methylbenzamide |
| 1592. | 1-{3-[(2-{3-[(4-methanesulfonyl-2-methoxyphenyl)amino]prop-1-yn-1-yl}-1- |
| | (2,2,2-trifluoroethyl)-1H-indol-4-yl)amino]pyrrolidin-1-yl}-3-methoxypropan-2-ol |
| 1593. | N-[3-(4-{[1-(2-hydroxy-3-methoxypropyl)pyrrolidin-3-yl]amino}-1-(2,2,2- |
| | trifluoroethyl)-1H-indol-2-yl)prop-2-yn-1-yl]benzamide |
| 1594. | 1-{3-[(2-{3-[(4-methanesulfonylphenyl)amino]prop-1-yn-1-yl}-1-(2,2,2- |
| | trifluoroethyl)-1H-indol-4-yl)amino]pyrrolidin-1-yl}-3-methoxypropan-2-ol |
| 1595. | 2-{3-[(4-methanesulfonyl-2-methoxyphenyl)amino]prop-1-yn-1-yl}-N-[1-(oxan- |
| | 4-yl)pyrrolidin-3-yl]-1-(2,2,2-trifluoroethyl)-1H-indol-4-amine |
| 1596. | 2-(3-{[2-methoxy-4-(morpholine-4-sulfonyl)phenyl]amino}prop-1-yn-1-yl)-N- |
| | (piperidin-4-yl)-1-(2,2,2-trifluoroethyl)-1H-indol-4-amine |
| 1597. | 3-methoxy-N,N-dimethyl-4-[(3-{4-[(piperidin-4-yl)amino]-1-(2,2,2- |
| | trifluoroethyl)-1H-indol-2-yl}prop-2-yn-1-yl)amino]benzene-1-sulfonamide |
| 1598. | 2-{5-methanesulfonyl-2-[(3-{4-[(piperidin-4-yl)amino]-1-(2,2,2-trifluoroethyl)- |
| | 1H-indol-2-yl}prop-2-yn-1-yl)amino]phenoxy}acetonitrile |
| 1599. | 3-methoxy-4-((3-(4-(piperidin-4-ylamino)-1-(2,2,2-trifluoroethyl)-1H-indol-2- |
| | yl)prop-2-yn-1-yl)amino)benzamide |
| 1600. | 2-{3-[(4-methanesulfonyl-2-methoxyphenyl)amino]prop-1-yn-1-yl}-N-[(3R)- |
| | piperidin-3-yl]-1-(2,2,2-trifluoroethyl)-1H-indol-4-amine |
| 1601. | 3-methoxy-4-{[3-(4-{[(2S,4S)-2-methylpiperidin-4-yl]amino}-1-(2,2,2- |
| | trifluoroethyl)-1H-indol-2-yl)prop-2-yn-1-yl]amino}benzene-1-sulfonamide |
| 1602. | 3-methoxy-4-((3-(4-((1-methylpiperidin-4-yl)amino)-1-(2,2,2-trifluoroethyl)-1H- |
| | indol-2-yl)prop-2-yn-1-yl)amino)benzenesulfonamide |
| 1603. | 2-(3-{[4-methanesulfonyl-2-(trifluoromethoxy)phenyl]amino}prop-1-yn-1-yl)-N- |
| | (1-methylpiperidin-4-yl)-1-(2,2,2-trifluoroethyl)-1H-indol-4-amine |
| 1604. | N-(1-methylpiperidin-4-yl)-2-(3-((4-(methylsulfonyl)-2-(2,2,2- |
| | trifluoroethoxy)phenyl)amino)prop-1-yn-1-yl)-1-(2,2,2-trifluoroethyl)-1H-indol-4- |
| | amine |
| 1605. | 2-{4-methoxy-5-[(3-{4-[(1-methylpiperidin-4-yl)amino]-1-(2,2,2-trifluoroethyl)- |
| | 1H-indol-2-yl}prop-2-yn-1-yl)amino]pyridin-2-yl}-2-methylpropanenitrile |
| 1606. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]-N-(1,2-oxazol-3-yl)benzene-1-sulfonamide |
| 1607. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]-N-(oxan-4-yl)benzene-1-sulfonamide |
| 1608. | 3-methoxy-N-(5-methyl-1,2-oxazol-3-yl)-4-[(3-{4-[(1-methylpiperidin-4- |
| | yl)amino]-1-(2,2,2-trifluoroethyl)-1H-indol-2-yl}prop-2-yn-1-yl)amino]benzene- |
| | 1-sulfonamide |
| 1609. | N-(2-hydroxyethyl)-3-methoxy-N-methyl-4-[(3-{4-[(1-methylpiperidin-4- |
| | yl)amino]-1-(2,2,2-trifluoroethyl)-1H-indol-2-yl}prop-2-yn-1-yl)amino]benzene- |
| | 1-sulfonamide |
| 1610. | 3-methoxy-N,N-dimethyl-4-[(3-{4-[(1-methylpiperidin-4-yl)amino]-1-(2,2,2- |
| | trifluoroethyl)-1H-indol-2-yl}prop-2-yn-1-yl)amino]benzene-1-sulfonamide |
| 1611. | 4-[(3-{6-fluoro-4-[(1-methylpiperidin-4-yl)amino]-1-(2,2,2-trifluoroethyl)-1H- |
| | indol-2-yl}prop-2-yn-1-yl)amino]-3-methoxybenzene-1-sulfonamide |
| 1612. | 6-fluoro-2-{3-[(4-methanesulfonyl-2-methoxyphenyl)amino]prop-1-yn-1-yl}-N- |
| | (1-methylpiperidin-4-yl)-1-(2,2,2-trifluoroethyl)-1H-indol-4-amine |
| 1613. | 2-(3-((4-methoxy-6-(methylsulfonyl)pyridin-3-yl)amino)prop-1-yn-1-yl)-N-(1- |
| | methylpiperidin-4-yl)-1-(2,2,2-trifluoroethyl)-1H-indol-4-amine |
| 1614. | 3-methoxy-N-(2-methoxyethyl)-N-methyl-4-[(3-{4-[(1-methylpiperidin-4- |
| | yl)amino]-1-(2,2,2-trifluoroethyl)-1H-indol-2-yl}prop-2-yn-1-yl)amino]benzene- |
| | 1-sulfonamide |
| 1615. | 2-{3-[(4-methanesulfonyl-2-methoxyphenyl)(methyl)amino]prop-1-yn-1-yl}-N- |
| | (1-methylpiperidin-4-yl)-1-(2,2,2-trifluoroethyl)-1H-indol-4-amine |
| 1616. | 1-(4-{3-methoxy-4-[(3-{4-[(1-methylpiperidin-4-yl)amino]-1-(2,2,2- |
| | trifluoroethyl)-1H-indol-2-yl}prop-2-yn-1-yl)amino]benzenesulfonyl}piperazin-1- |
| | yl)ethan-1-one |
| 1617. | 2-(3-{[2-methoxy-4-(morpholine-4-sulfonyl)phenyl]amino}prop-1-yn-1-yl)-N-(1- |
| | methylpiperidin-4-yl)-1-(2,2,2-trifluoroethyl)-1H-indol-4-amine |
| 1618. | 3-methoxy-4-((3-(4-((1-methylpiperidin-4-yl)amino)-1-(2,2,2-trifluoroethyl)-1H- |
| | indol-2-yl)prop-2-yn-1-yl)amino)benzoic acid |
| 1619. | 3-methoxy-N-methyl-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1620. | N,N-bis(2-hydroxyethyl)-3-methoxy-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1621. | 3-methoxy-N-methyl-4-[(3-{4-[(1-methylpiperidin-4-yl)amino]-1-(2,2,2- |
| | trifluoroethyl)-1H-indol-2-yl}prop-2-yn-1-yl)amino]benzene-1-sulfonamide |
| 1622. | 2-[3-({2-methoxy-4-[(4-methylpiperazin-1-yl)sulfonyl]phenyl}amino)prop-1-yn- |
| | 1-yl]-N-(1-methylpiperidin-4-yl)-1-(2,2,2-trifluoroethyl)-1H-indol-4-amine |
| 1623. | 5-methanesulfonyl-2-[(3-{4-[(1-methylpiperidin-4-yl)amino]-1-(2,2,2- |
| | trifluoroethyl)-1H-indol-2-yl}prop-2-yn-1-yl)amino]phenol |
| 1624. | 2-{3-[(4-methanesulfonyl-2-methoxyphenyl)amino]prop-1-yn-1-yl}-6-methoxy- |
| | N-(1-methylpiperidin-4-yl)-1-(2,2,2-trifluoroethyl)-1H-indol-4-amine |
| 1625. | 2-{3-[2-(dimethylamino)-4-methanesulfonylphenoxy]prop-1-yn-1-yl}-N-(1- |
| | methylpiperidin-4-yl)-1-(2,2,2-trifluoroethyl)-1H-indol-4-amine |
| 1626. | 2-{5-methanesulfonyl-2-[(3-{4-[(1-methylpiperidin-4-yl)amino]-1-(2,2,2- |
| | trifluoroethyl)-1H-indol-2-yl}prop-2-yn-1-yl)amino]phenoxy}acetonitrile |
| 1627. | 2-(3-{[2-(fluoromethoxy)-4-methanesulfonylphenyl]amino}prop-1-yn-1-yl)-N-(1- |
| | methylpiperidin-4-yl)-1-(2,2,2-trifluoroethyl)-1H-indol-4-amine |
| 1628. | 2-(3-{[2-methoxy-4-(morpholine-4-carbonyl)phenyl]amino}prop-1-yn-1-yl)-N-(1- |
| | methylpiperidin-4-yl)-1-(2,2,2-trifluoroethyl)-1H-indol-4-amine |
| 1629. | 1-{3-methoxy-4-[(3-{4-[(1-methylpiperidin-4-yl)amino]-1-(2,2,2-trifluoroethyl)- |
| | 1H-indol-2-yl}prop-2-yn-1-yl)amino]benzoyl}piperidin-4-ol |
| 1630. | 3-(3-(4-((1-methylpiperidin-4-yl)amino)-1-(2,2,2-trifluoroethyl)-1H-indol-2- |
| | yl)prop-2-yn-1-yl)-6-(methylsulfonyl)benzo[d]oxazol-2(3H)-one |
| 1631. | 2-(3-{[2-methoxy-4-(5-methoxypyridin-3-yl)phenyl]amino}prop-1-yn-1-yl)-N-(1- |
| | methylpiperidin-4-yl)-1-(2,2,2-trifluoroethyl)-1H-indol-4-amine |
| 1632. | 2-{3-[(5-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1633. | N-(2-hydroxyethyl)-3-methoxy-4-[(3-{4-[(1-methylpiperidin-4-yl)amino]-1- |
| | (2,2,2-trifluoroethyl)-1H-indol-2-yl}prop-2-yn-1-yl)amino]benzamide |
| 1634. | 3-methoxy-N-(2-methoxyethyl)-4-[(3-{4-[(1-methylpiperidin-4-yl)amino]-1- |
| | (2,2,2-trifluoroethyl)-1H-indol-2-yl}prop-2-yn-1-yl)amino]benzamide |
| 1635. | 3-methoxy-N-(1-methylpiperidin-4-yl)-4-[(3-{4-[(1-methylpiperidin-4-yl)amino]- |
| | 1-(2,2,2-trifluoroethyl)-1H-indol-2-yl}prop-2-yn-1-yl)amino]benzamide |
| 1636. | 2-[3-({4-[4-(dimethylamino)piperidine-1-carbonyl]-2- |
| | methoxyphenyl}amino)prop-1-yn-1-yl]-N-(1-methylpiperidin-4-yl)-1-(2,2,2- |
| | trifluoroethyl)-1H-indol-4-amine |
| 1637. | 3-methoxy-4-[(3-{4-[(1-methylpiperidin-4-yl)amino]-1-(2,2,2-trifluoroethyl)-1H- |
| | indol-2-yl}prop-2-yn-1-yl)amino]-N-(oxan-4-yl)benzamide |
| 1638. | 2-(3-{[2-methoxy-4-(4-methylpiperazine-1-carbonyl)phenyl]amino}prop-1-yn-1- |
| | yl)-N-(1-methylpiperidin-4-yl)-1-(2,2,2-trifluoroethyl)-1H-indol-4-amine |
| 1639. | 2-{3-[(2-methoxy-4-{2-oxa-6-azaspiro[3.3]heptane-6- |
| | carbonyl}phenyl)amino]prop-1-yn-1-yl}-N-(1-methylpiperidin-4-yl)-1-(2,2,2- |
| | trifluoroethyl)-1H-indol-4-amine |
| 1640. | 2-(3-{[2-methoxy-4-(pyridin-3-yl)phenyl]amino}prop-1-yn-1-yl)-N-(1- |
| | methylpiperidin-4-yl)-1-(2,2,2-trifluoroethyl)-1H-indol-4-amine |
| 1641. | 2-(3-{[2-methoxy-4-(pyridin-4-yl)phenyl]amino}prop-1-yn-1-yl)-N-(1- |
| | methylpiperidin-4-yl)-1-(2,2,2-trifluoroethyl)-1H-indol-4-amine |
| 1642. | N-(3-{4-[(1-methylpiperidin-4-yl)amino]-1-(2,2,2-trifluoroethyl)-1H-indol-2- |
| | yl}prop-2-yn-1-yl)pyridine-3-carboxamide |
| 1643. | 2-(3-{[2-methoxy-4-(1,3-oxazol-2-yl)phenyl]amino}prop-1-yn-1-yl)-N-(1- |
| | methylpiperidin-4-yl)-1-(2,2,2-trifluoroethyl)-1H-indol-4-amine |
| 1644. | 2-{3-[(3-methanesulfonylphenyl)amino]prop-1-yn-1-yl}-N-(1-methylpiperidin-4- |
| | yl)-1-(2,2,2-trifluoroethyl)-1H-indol-4-amine |
| 1645. | N-(1-methylpiperidin-4-yl)-2-[3-({4-[(morpholin-4- |
| | yl)methyl]phenyl}amino)prop-1-yn-1-yl]-1-(2,2,2-trifluoroethyl)-1H-indol-4- |
| | amine |
| 1646. | 2-(3-{[2-methoxy-4-(1,3-thiazol-2-yl)phenyl]amino}prop-1-yn-1-yl)-N-(1- |
| | methylpiperidin-4-yl)-1-(2,2,2-trifluoroethyl)-1H-indol-4-amine |
| 1647. | 2-[3-({2-methoxy-4-[1-(2-methoxyethyl)-1H-pyrazol-4-yl]phenyl}amino)prop-1- |
| | yn-1-yl]-N-(1-methylpiperidin-4-yl)-1-(2,2,2-trifluoroethyl)-1H-indol-4-amine |
| 1648. | 2-{3-[(2-methoxy-4-{7-oxa-2-azaspiro[3.5]nonane-2- |
| | carbonyl}phenyl)amino]prop-1-yn-1-yl}-N-(1-methylpiperidin-4-yl)-1-(2,2,2- |
| | trifluoroethyl)-1H-indol-4-amine |
| 1649. | 2-{3-[(4-chloro-2-methoxyphenyl)amino]prop-1-yn-1-yl}-N-(1-methylpiperidin- |
| | 4-yl)-1-(2,2,2-trifluoroethyl)-1H-indol-4-amine |
| 1650. | 2-(3-(((3S,4R)-3-methoxytetrahydro-2H-pyran-4-yl)amino)prop-1-yn-1-yl)-N-(1- |
| | methylpiperidin-4-yl)-1-(2,2,2-trifluoroethyl)-1H-indol-4-amine |
| 1651. | 2-fluoro-5-methoxy-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1652. | 2-(3-{[2-methoxy-4-(trifluoromethyl)phenyl]amino}prop-1-yn-1-yl)-N-(1- |
| | methylpiperidin-4-yl)-1-(2,2,2-trifluoroethyl)-1H-indol-4-amine |
| 1653. | 2-fluoro-5-methoxy-N-methyl-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1654. | 2-{3-[(4-methanesulfonyl-2-methoxyphenyl)amino]prop-1-yn-1-yl}-N-(1- |
| | methylpiperidin-4-yl)-1-[(oxiran-2-yl)methyl]-1H-indol-4-amine |
| 1655. | 2-(3-((2-ethoxy-4-(methylsulfonyl)phenyl)amino)prop-1-yn-1-yl)-N-(1- |
| | methylpiperidin-4-yl)-1-(2,2,2-trifluoroethyl)-1H-indol-4-amine |
| 1656. | 2-{3-[(2-fluoro-4-methanesulfonyl-6-methoxyphenyl)amino]prop-1-yn-1-yl}-N- |
| | (1-methylpiperidin-4-yl)-1-(2,2,2-trifluoroethyl)-1H-indol-4-amine |
| 1657. | 2-{3-[(4-methanesulfonyl-2-methoxyphenyl)amino]prop-1-yn-1-yl}-3-methyl-N- |
| | (1-methylpiperidin-4-yl)-1-(2,2,2-trifluoroethyl)-1H-indol-4-amine |
| 1658. | 2-(3-(((3R,4R)-3-methoxytetrahydro-2H-pyran-4-yl)amino)prop-1-yn-1-yl)-N-(1- |
| | methylpiperidin-4-yl)-1-(2,2,2-trifluoroethyl)-1H-indol-4-amine |
| 1659. | 2-{3-[(5-fluoro-4-methanesulfonyl-2-methoxyphenyl)amino]prop-1-yn-1-yl}-N- |
| | (1-methylpiperidin-4-yl)-1-(2,2,2-trifluoroethyl)-1H-indol-4-amine |
| 1660. | 2-{3-[(5-methanesulfonylthiophen-2-yl)amino]prop-1-yn-1-yl}-N-(1- |
| | methylpiperidin-4-yl)-1-(2,2,2-trifluoroethyl)-1H-indol-4-amine |
| 1661. | N-methyl-5-[(3-{4-[(1-methylpiperidin-4-yl)amino]-1-(2,2,2-trifluoroethyl)-1H- |
| | indol-2-yl}prop-2-yn-1-yl)amino]thiophene-2-carboxamide |
| 1662. | N,N-dimethyl-5-[(3-{4-[(1-methylpiperidin-4-yl)amino]-1-(2,2,2-trifluoroethyl)- |
| | 1H-indol-2-yl}prop-2-yn-1-yl)amino]thiophene-2-carboxamide |
| 1663. | 5-[(3-{4-[(1-methylpiperidin-4-yl)amino]-1-(2,2,2-trifluoroethyl)-1H-indol-2- |
| | yl}prop-2-yn-1-yl)amino]thiophene-2-carboxylic acid |
| 1664. | 2-(3-((4-methoxypyridin-3-yl)amino)prop-1-yn-1-yl)-N-(1-methylpiperidin-4-yl)- |
| | 1-(2,2,2-trifluoroethyl)-1H-indol-4-amine |
| 1665. | 2-(2-((3-(4-((1-methylpiperidin-4-yl)amino)-1-(2,2,2-trifluoroethyl)-1H-indol-2- |
| | yl)prop-2-yn-1-yl)amino)-5-(methylsulfonyl)phenoxy)acetamide |
| 1666. | N-(2-hydroxyethyl)-3-methoxy-4-((3-(4-((1-methylpiperidin-4-yl)amino)-1-(2,2,2- |
| | trifluoroethyl)-1H-indol-2-yl)prop-2-yn-1-yl)amino)benzenesulfonamide |
| 1667. | 4-((2-(3-((2-methoxy-4-(methylsulfonyl)phenyl)amino)prop-1-yn-1-yl)-1-(2,2,2- |
| | trifluoroethyl)-1H-indol-4-yl)amino)-1-methylpiperidin-2-one |
| 1668. | 2-{3-[(4-methanesulfonyl-2-methoxyphenyl)amino]prop-1-yn-1-yl}-N-[(3R,4S)- |
| | 3-methoxy-1-methylpiperidin-4-yl]-1-(2,2,2-trifluoroethyl)-1H-indol-4-amine |
| 1669. | 2-{3-[(4-methanesulfonyl-2-methoxyphenyl)amino]prop-1-yn-1-yl}-N-[(3S,4R)- |
| | 3-methoxy-1-methylpiperidin-4-yl]-1-(2,2,2-trifluoroethyl)-1H-indol-4-amine |
| 1670. | 2-{3-[(4-methanesulfonyl-2-methoxyphenyl)amino]prop-1-yn-1-yl}-N-[(3S,4R)- |
| | 3-methoxy-1-methylpiperidin-4-yl]-N-methyl-1-(2,2,2-trifluoroethyl)-1H-indol-4- |
| | amine |
| 1671. | N-(1-ethylpiperidin-4-yl)-2-(3-{[2-(fluoromethoxy)-4- |
| | methanesulfonylphenyl]amino}prop-1-yn-1-yl)-1-(2,2,2-trifluoroethyl)-1H-indol- |
| | 4-amine |
| 1672. | 4-[(3-{4-[(1-ethylpiperidin-4-yl)amino]-1-(2,2,2-trifluoroethyl)-1H-indol-2- |
| | yl}prop-2-yn-1-yl)amino]-3-methoxy-N-methylbenzamide |
| 1673. | 2-{2-[(3-{4-[(1-ethylpiperidin-4-yl)amino]-1-(2,2,2-trifluoroethyl)-1H-indol-2- |
| | yl}prop-2-yn-1-yl)amino]-5-methanesulfonylphenoxy}acetonitrile |
| 1674. | N-(1-ethylpiperidin-4-yl)-2-{3-[(4-methanesulfonyl-2- |
| | methoxyphenyl)amino]prop-1-yn-1-yl}-1-(2,2,2-trifluoroethyl)-1H-indol-4-amine |
| 1675. | 4-[(3-{4-[(1-ethylpiperidin-4-yl)amino]-1-(2,2,2-trifluoroethyl)-1H-indol-2- |
| | yl}prop-2-yn-1-yl)amino]-3-methoxybenzene-1-sulfonamide |
| 1676. | 3-methoxy-N-methyl-4-{[3-(4-{[1-(propan-2-yl)piperidin-4-yl]amino}-1-(2,2,2- |
| | trifluoroethyl)-1H-indol-2-yl)prop-2-yn-1-yl]amino}benzamide |
| 1677. | 2-(5-methanesulfonyl-2-{[3-(4-{[1-(propan-2-yl)piperidin-4-yl]amino}-1-(2,2,2- |
| | trifluoroethyl)-1H-indol-2-yl)prop-2-yn-1-yl]amino}phenoxy)acetonitrile |
| 1678. | 2-(3-{[2-(fluoromethoxy)-4-methanesulfonylphenyl]amino}prop-1-yn-1-yl)-N-[1- |
| | (propan-2-yl)piperidin-4-yl]-1-(2,2,2-trifluoroethyl)-1H-indol-4-amine |
| 1679. | 2-{3-[(4-methanesulfonyl-2-methoxyphenyl)amino]prop-1-yn-1-yl}-N-[1- |
| | (propan-2-yl)piperidin-4-yl]-1-(2,2,2-trifluoroethyl)-1H-indol-4-amine |
| 1680. | 3-methoxy-4-{[3-(4-{[1-(propan-2-yl)piperidin-4-yl]amino}-1-(2,2,2- |
| | trifluoroethyl)-1H-indol-2-yl)prop-2-yn-1-yl]amino}benzene-1-sulfonamide |
| 1681. | 2-[2-(2-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}ethoxy)ethoxy]ethan- |
| | 1-ol |
| 1682. | 4-({4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}methyl)-1,3-dioxolan-2- |
| | one |
| 1683. | 3-methoxy-4-({3-[4-({1-[(2-oxo-1,3-dioxolan-4-yl)methyl]piperidin-4-yl}amino)- |
| | 1-(2,2,2-trifluoroethyl)-1H-indol-2-yl]prop-2-yn-1-yl}amino)benzene-1- |
| | sulfonamide |
| 1684. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxy-N-(1,2-oxazol-3-yl)benzene-1- |
| | sulfonamide |
| 1685. | 4-((3-(4-((1-(2,3-dihydroxypropyl)piperidin-4-yl)amino)-1-(2,2,2-trifluoroethyl)- |
| | 1H-indol-2-yl)prop-2-yn-1-yl)amino)-3-methoxybenzenesulfonamide |
| 1686. | 4-({3-[4-({1-[(2S)-2,3-dihydroxypropyl]piperidin-4-yl}amino)-1-(2,2,2- |
| | trifluoroethyl)-1H-indol-2-yl]prop-2-yn-1-yl}amino)-3-methoxybenzene-1- |
| | sulfonamide |
| 1687. | 4-({3-[4-({1-[(2R)-2,3-dihydroxypropyl]piperidin-4-yl}amino)-1-(2,2,2- |
| | trifluoroethyl)-1H-indol-2-yl]prop-2-yn-1-yl}amino)-3-methoxybenzene-1- |
| | sulfonamide |
| 1688. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxy-N-(5-methyl-1,2-oxazol-3- |
| | yl)benzene-1-sulfonamide |
| 1689. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxy-N-(oxan-4-yl)benzene-1- |
| | sulfonamide |
| 1690. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-N-(2-hydroxyethyl)-3-methoxy-N- |
| | methylbenzene-1-sulfonamide |
| 1691. | 2-(5-{[3-(4-{[1-(2,3-dihydroxypropyl)piperidin-4-yl]amino}-1-(2,2,2- |
| | trifluoroethyl)-1H-indol-2-yl)prop-2-yn-1-yl]amino}-4-methoxypyridin-2-yl)-2- |
| | methylpropanenitrile |
| 1692. | N-(2-hydroxyethyl)-3-methoxy-N-methyl-4-{[3-(4-{[1-(oxan-4-yl)piperidin-4- |
| | yl]amino}-1-(2,2,2-trifluoroethyl)-1H-indol-2-yl)prop-2-yn-1-yl]amino}benzene- |
| | 1-sulfonamide |
| 1693. | 3-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)propane-1,2-diol |
| 1694. | (2R)-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}propane-1,2-diol |
| 1695. | (2S)-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}propane-1,2-diol |
| 1696. | 3-[4-({2-[3-({2-methoxy-4-[(4-methylpiperazin-1-yl)sulfonyl]phenyl}amino)prop- |
| | 1-yn-1-yl]-1-(2,2,2-trifluoroethyl)-1H-indol-4-yl}amino)piperidin-1-yl]propane- |
| | 1,2-diol |
| 1697. | 4-((3-(4-((1-(2,3-dihydroxypropyl)piperidin-4-yl)amino)-1-(2,2,2-trifluoroethyl)- |
| | 1H-indol-2-yl)prop-2-yn-1-yl)amino)-3-methoxybenzoic acid |
| 1698. | methyl 4-((3-(4-((1-(2,3-dihydroxypropyl)piperidin-4-yl)amino)-1-(2,2,2- |
| | trifluoroethyl)-1H-indol-2-yl)prop-2-yn-1-yl)amino)-3-methoxybenzoate |
| 1699. | 3-methoxy-4-[(3-{4-[(1-{[(4R)-2-oxo-1,3-dioxolan-4-yl]methyl}piperidin-4- |
| | yl)amino]-1-(2,2,2-trifluoroethyl)-1H-indol-2-yl}prop-2-yn-1-yl)amino]benzene- |
| | 1-sulfonamide |
| 1700. | (4R)-4-({4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}methyl)-1,3-dioxolan- |
| | 2-one |
| 1701. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxy-N,N-dimethylbenzene-1- |
| | sulfonamide |
| 1702. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}EtOAc |
| 1703. | N-(1-{[(4R)-2,2-dimethyl-1,3-dioxolan-4-yl]methyl}piperidin-4-yl)-2-{3-[(4- |
| | methanesulfonyl-2-methoxyphenyl)amino]prop-1-yn-1-yl}-1-(2,2,2- |
| | trifluoroethyl)-1H-indol-4-amine |
| 1704. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-2-yl acetate |
| 1705. | 4-[(3-{4-[(1-{[(4R)-2,2-dimethyl-1,3-dioxolan-4-yl]methyl}piperidin-4- |
| | yl)amino]-1-(2,2,2-trifluoroethyl)-1H-indol-2-yl}prop-2-yn-1-yl)amino]-3- |
| | methoxybenzene-1-sulfonamide |
| 1706. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1707. | 1-(4-((2-(3-((4-(ethylsulfonyl)-2-methoxyphenyl)amino)prop-1-yn-1-yl)-1-(2,2,2- |
| | trifluoroethyl)-1H-indol-4-yl)amino)piperidin-1-yl)-3-methoxypropan-2-ol |
| 1708. | 1-ethoxy-3-(4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)propan-2-ol |
| 1709. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxy-N-(2-methoxyethyl)-N- |
| | methylbenzene-1-sulfonamide |
| 1710. | 1-(acetyloxy)-3-{4-[(2-{3-[(2-methoxy-4-sulfamoylphenyl)amino]prop-1-yn-1- |
| | yl}-1-(2,2,2-trifluoroethyl)-1H-indol-4-yl)amino]piperidin-1-yl}propan-2-yl |
| | acetate |
| 1711. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzene-1- |
| | sulfonamide |
| 1712. | 3-methoxy-4-{[3-(4-{[1-(2-methoxyacetyl)piperidin-4-yl]amino}-1-(2,2,2- |
| | trifluoroethyl)-1H-indol-2-yl)prop-2-yn-1-yl]amino}benzene-1-sulfonamide |
| 1713. | 1-(4-(N-(2-(3-((2-methoxy-4-(methylsulfonyl)phenyl)amino)prop-1-yn-1-yl)-1- |
| | (2,2,2-trifluoroethyl)-1H-indol-4-yl)acetamido)piperidin-1-yl)propan-2-yl acetate |
| 1714. | 1-[4-(4-{[3-(4-{[1-(2,3-dihydroxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)piperazin-1-yl]ethan-1-one |
| 1715. | (4S)-4-({4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}methyl)-1,3-dioxolan- |
| | 2-one |
| 1716. | 1-(acetyloxy)-3-{4-[(2-{3-[(4-methanesulfonyl-2-methoxyphenyl)amino]prop-1- |
| | yn-1-yl}-1-(2,2,2-trifluoroethyl)-1H-indol-4-yl)amino]piperidin-1-yl}propan-2-yl |
| | acetate |
| 1717. | N-[1-(2,3-dimethoxypropyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1718. | 4-{[3-(4-{[1-(2,3-dimethoxypropyl)piperidin-4-yl]amino}-1-(2,2,2-trifluoroethyl)- |
| | 1H-indol-2-yl)prop-2-yn-1-yl]amino}-3-methoxybenzene-1-sulfonamide |
| 1719. | 3-(4-{[2-(3-{[2-methoxy-4-(morpholine-4-sulfonyl)phenyl]amino}prop-1-yn-1- |
| | yl)-1-(2,2,2-trifluoroethyl)-1H-indol-4-yl]amino}piperidin-1-yl)propane-1,2-diol |
| 1720. | 4-({3-[4-({1-[(2R)-2,3-dihydroxypropyl]piperidin-4-yl}amino)-1-(2,2,2- |
| | trifluoroethyl)-1H-indol-2-yl]prop-2-yn-1-yl}amino)-3-methoxybenzamide |
| 1721. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethyl 2- |
| | methylpropanoate |
| 1722. | 2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethyl propanoate |
| 1723. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3- |
| | (propanoyloxy)propan-2-yl propanoate |
| 1724. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}-3-(propanoyloxy)propan-2-yl |
| | propanoate |
| 1725. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-[(2- |
| | methylpropanoyl)oxy]propan-2-yl 2-methylpropanoate |
| 1726. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}-3-[(2- |
| | methylpropanoyl)oxy]propan-2-yl 2-methylpropanoate |
| 1727. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-2-yl propanoate |
| 1728. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-2-yl 2- |
| | methylpropanoate |
| 1729. | 2-hydroxy-3-{4-[(2-{3-[(4-methanesulfonyl-2-methoxyphenyl)amino]prop-1-yn- |
| | 1-yl}-1-(2,2,2-trifluoroethyl)-1H-indol-4-yl)amino]piperidin-1-yl}propyl 2- |
| | methylpropanoate |
| 1730. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-yl |
| | acetate |
| 1731. | 2-hydroxy-3-{4-[(2-{3-[(4-methanesulfonyl-2-methoxyphenyl)amino]prop-1-yn- |
| | 1-yl}-1-(2,2,2-trifluoroethyl)-1H-indol-4-yl)amino]piperidin-1-yl}propyl |
| | propanoate |
| 1732. | N,N-bis(2-hydroxyethyl)-4-{[3-(4-{[1-(2-hydroxypropyl)piperidin-4-yl]amino}-1- |
| | (2,2,2-trifluoroethyl)-1H-indol-2-yl)prop-2-yn-1-yl]amino}-3-methoxybenzene-1- |
| | sulfonamide |
| 1733. | 4-{[3-(4-{[1-(2-hydroxypropyl)piperidin-4-yl]amino}-1-(2,2,2-trifluoroethyl)-1H- |
| | indol-2-yl)prop-2-yn-1-yl]amino}-3-methoxy-N-methylbenzamide |
| 1734. | (S)-4-((3-(4-((1-(2-hydroxy-3-methoxypropyl)piperidin-4-yl)amino)-1-(2,2,2- |
| | trifluoroethyl)-1H-indol-2-yl)prop-2-yn-1-yl)amino)-3-methoxy-N- |
| | methylbenzamide |
| 1735. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl 2- |
| | methylpropanoate |
| 1736. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl acetate |
| 1737. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}ethyl 2-methylpropanoate |
| 1738. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxy-N- |
| | methylbenzamide |
| 1739. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-yl |
| | propanoate |
| 1740. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-yl |
| | 2-methylpropanoate |
| 1741. | 1-methoxy-3-{4-[(2-{3-[(2-methoxy-4-sulfamoylphenyl)amino]prop-1-yn-1-yl}- |
| | 1-(2,2,2-trifluoroethyl)-1H-indol-4-yl)amino]piperidin-1-yl}propan-2-yl 2- |
| | methylpropanoate |
| 1742. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl propanoate |
| 1743. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)acetamide |
| 1744. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)propanamide |
| 1745. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}ethyl propanoate |
| 1746. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxy-N- |
| | methylbenzene-1-sulfonamide |
| 1747. | 1-methoxy-3-{4-[(2-{3-[(2-methoxy-4-sulfamoylphenyl)amino]prop-1-yn-1-yl}- |
| | 1-(2,2,2-trifluoroethyl)-1H-indol-4-yl)amino]piperidin-1-yl}propan-2-yl |
| | propanoate |
| 1748. | (2R)-1-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-3-methoxypropan-2- |
| | ol |
| 1749. | (2S)-1-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-3-methoxypropan-2- |
| | ol |
| 1750. | 3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1751. | 3-methoxy-4-((3-(4-((1-(2-methoxyethyl)piperidin-4-yl)amino)-1-(2,2,2- |
| | trifluoroethyl)-1H-indol-2-yl)prop-2-yn-1-yl)amino)benzenesulfonamide |
| 1752. | 2-(3-((2-methoxy-4-(methylsulfonyl)phenyl)amino)prop-1-yn-1-yl)-N-(1-(2- |
| | methoxyethyl)piperidin-4-yl)-1-(2,2,2-trifluoroethyl)-1H-indol-4-amine |
| 1753. | 3-methoxy-4-{[3-(4-{[1-(2-methoxyethyl)piperidin-4-yl]amino}-1-(2,2,2- |
| | trifluoroethyl)-1H-indol-2-yl)prop-2-yn-1-yl]amino}-N-methylbenzamide |
| 1754. | 3-methoxy-4-{[3-(4-{[1-(2-methoxyethyl)piperidin-4-yl]amino}-1-(2,2,2- |
| | trifluoroethyl)-1H-indol-2-yl)prop-2-yn-1-yl]amino}-N-methylbenzene-1- |
| | sulfonamide |
| 1755. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}EtOAc |
| 1756. | 1-methoxy-3-{4-[(2-{3-[(2-methoxy-4-sulfamoylphenyl)amino]prop-1-yn-1-yl}- |
| | 1-(2,2,2-trifluoroethyl)-1H-indol-4-yl)amino]piperidin-1-yl}propan-2-yl acetate |
| 1757. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzamide |
| 1758. | 2-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5-methanesulfonylphenol |
| 1759. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxy-N,N- |
| | dimethylbenzene-1-sulfonamide |
| 1760. | 4-({3-[4-({1-[(2R)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxybenzene-1- |
| | sulfonamide |
| 1761. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzenesulfonyl)- |
| | N-methylpropanamide |
| 1762. | 1-(4-{[2-(3-{[2-(2-fluoroethoxy)-4-methanesulfonylphenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1763. | 1-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)tetradecan-1-one |
| 1764. | 1-methoxy-3-(4-{[2-(3-{[2-methoxy-4-(propanamidosulfonyl)phenyl]amino}prop- |
| | 1-yn-1-yl)-1-(2,2,2-trifluoroethyl)-1H-indol-4-yl]amino}piperidin-1-yl)propan-2- |
| | yl propanoate |
| 1765. | 1-(4-{[2-(3-{[2-methoxy-4-(propanamidosulfonyl)phenyl]amino}prop-1-yn-1-yl)- |
| | 1-(2,2,2-trifluoroethyl)-1H-indol-4-yl]amino}piperidin-1-yl)-3- |
| | (propanoyloxy)propan-2-yl propanoate |
| 1766. | (2R)-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-2-methoxypropan-1- |
| | ol |
| 1767. | (2S)-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-2-methoxypropan-1- |
| | ol |
| 1768. | 1-{4-[(2-{3-[(2-ethoxy-4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1769. | 4-({3-[4-({1-[(2S)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxybenzene-1- |
| | sulfonamide |
| 1770. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxy-N-(propan-2- |
| | yl)benzamide |
| 1771. | 3-hydroxy-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}benzene-1-sulfonamide |
| 1772. | (2R)-1-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-3-methoxypropan-2- |
| | yl 2-methylpropanoate |
| 1773. | N-(3-(4-((1-(2-hydroxy-3-methoxypropyl)piperidin-4-yl)amino)-1-(2,2,2- |
| | trifluoroethyl)-1H-indol-2-yl)prop-2-yn-1-yl)-N-(2-hydroxy-4-(N- |
| | propionylsulfamoyl)phenyl)propionamide |
| 1774. | 1-(4-{[2-(3-{[4-methanesulfonyl-2-(2-methoxyethoxy)phenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1775. | (2S)-1-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-3-methoxypropan-2- |
| | yl 2-methylpropanoate |
| 1776. | N-(3-(4-((1-(2-hydroxy-3-methoxypropyl)piperidin-4-yl)amino)-1-(2,2,2- |
| | trifluoroethyl)-1H-indol-2-yl)prop-2-yn-1-yl)-N-(2-hydroxy-4- |
| | (methylsulfonyl)phenyl)acetamide |
| 1777. | N-(3-(4-((1-(2-hydroxy-3-methoxypropyl)piperidin-4-yl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 1778. | N-(3-(4-((1-(2-hydroxy-3-methoxypropyl)piperidin-4-yl)amino)-1-(2,2,2- |
| | trifluoroethyl)-1H-indol-2-yl)prop-2-yn-1-yl)-N-(2-hydroxy-4- |
| | (methylsulfonyl)phenyl)isobutyramide |
| 1779. | 2-(2-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5- |
| | methanesulfonylphenoxy)acetonitrile |
| 1780. | 1-{4-[(2-{3-[(4-methanesulfonyl-2-methoxyphenyl)amino]prop-1-yn-1-yl}-5- |
| | methoxy-1-(2,2,2-trifluoroethyl)-1H-indol-4-yl)amino]piperidin-1-yl}-3- |
| | methoxypropan-2-ol |
| 1781. | 1-(4-{[2-(3-{[2-(2-hydroxyethoxy)-4-methanesulfonylphenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1782. | 1-(4-{[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]amino}piperidin-1-yl)-3- |
| | methoxypropan-2-ol |
| 1783. | 1-[4-({2-[3-(2-amino-4-methanesulfonylphenoxy)prop-1-yn-1-yl]-1-(2,2,2- |
| | trifluoroethyl)-1H-indol-4-yl}amino)piperidin-1-yl]-3-methoxypropan-2-ol |
| 1784. | 4-({3-[4-({1-[(2R)-2-hydroxy-3-methoxypropyl]piperidin-4-yl}amino)-1-(2,2,2- |
| | trifluoroethyl)-1H-indol-2-yl]prop-2-yn-1-yl}amino)-3-methoxy-N- |
| | methylbenzamide |
| 1785. | 4-({3-[4-({1-[(2S)-2-hydroxy-3-methoxypropyl]piperidin-4-yl}amino)-1-(2,2,2- |
| | trifluoroethyl)-1H-indol-2-yl]prop-2-yn-1-yl}amino)-3-methoxy-N- |
| | methylbenzamide |
| 1786. | 4-({3-[4-({1-[(2R)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxy-N- |
| | methylbenzamide |
| 1787. | 4-({3-[4-({1-[(2S)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxy-N- |
| | methylbenzamide |
| 1788. | 1-{4-[(2-{3-[4-methanesulfonyl-2-(methylamino)phenoxy]prop-1-yn-1-yl}-1- |
| | (2,2,2-trifluoroethyl)-1H-indol-4-yl)amino]piperidin-1-yl}-3-methoxypropan-2-ol |
| 1789. | 3-(2-fluoroethoxy)-4-{[3-(4-{[1-(2-hydroxy-3-methoxypropyl)piperidin-4- |
| | yl]amino}-1-(2,2,2-trifluoroethyl)-1H-indol-2-yl)prop-2-yn-1-yl]amino}-N- |
| | methylbenzamide |
| 1790. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-(2-methoxyethoxy)-N- |
| | methylbenzamide |
| 1791. | 3-(cyanomethoxy)-4-{[3-(4-{[1-(2-hydroxy-3-methoxypropyl)piperidin-4- |
| | yl]amino}-1-(2,2,2-trifluoroethyl)-1H-indol-2-yl)prop-2-yn-1-yl]amino}-N- |
| | methylbenzamide |
| 1792. | N-ethyl-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-3-methoxybenzamide |
| 1793. | 1-(4-{[2-(3-{[2-methoxy-4-(methylcarbamoyl)phenyl]amino}prop-1-yn-1-yl)-1- |
| | (2,2,2-trifluoroethyl)-1H-indol-4-yl]amino}piperidin-1-yl)-3- |
| | (propanoyloxy)propan-2-yl propanoate |
| 1794. | 1-methoxy-3-(4-{[2-(3-{[2-methoxy-4-(methylcarbamoyl)phenyl]amino}prop-1- |
| | yn-1-yl)-1-(2,2,2-trifluoroethyl)-1H-indol-4-yl]amino}piperidin-1-yl)propan-2-yl |
| | propanoate |
| 1795. | 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxy-N-methylbenzamide |
| 1796. | 3-(2-cyanoethoxy)-4-{[3-(4-{[1-(2-hydroxy-3-methoxypropyl)piperidin-4- |
| | yl]amino}-1-(2,2,2-trifluoroethyl)-1H-indol-2-yl)prop-2-yn-1-yl]amino}-N- |
| | methylbenzamide |
| 1797. | 1-ethoxy-3-{4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl}propan-2-ol |
| 1798. | 2-(2-{[3-(4-{[1-(2-hydroxypropyl)piperidin-4-yl]amino}-1-(2,2,2-trifluoroethyl)- |
| | 1H-indol-2-yl)prop-2-yn-1-yl]amino}-5-methanesulfonylphenoxy)acetonitrile |
| 1799. | 1-(4-{[2-(3-{[4-(ethanesulfonyl)-2-methoxyphenyl]amino}prop-1-yn-1-yl)-1- |
| | (2,2,2-trifluoroethyl)-1H-indol-4-yl]amino}piperidin-1-yl)-3-methoxypropan-2-ol |
| 1800. | 3-(fluoromethoxy)-4-{[3-(4-{[1-(2-hydroxy-3-methoxypropyl)piperidin-4- |
| | yl]amino}-1-(2,2,2-trifluoroethyl)-1H-indol-2-yl)prop-2-yn-1-yl]amino}-N- |
| | methylbenzamide |
| 1801. | 1-(4-{[2-(3-{[2-(fluoromethoxy)-4-methanesulfonylphenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1802. | 2-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxyphenyl)-2- |
| | methylpropanenitrile |
| 1803. | (2S)-1-(4-{[2-(3-{[2-(fluoromethoxy)-4-methanesulfonylphenyl]amino}prop-1- |
| | yn-1-yl)-1-(2,2,2-trifluoroethyl)-1H-indol-4-yl]amino}piperidin-1-yl)-3- |
| | methoxypropan-2-ol |
| 1804. | (2R)-1-(4-{[2-(3-{[2-(fluoromethoxy)-4-methanesulfonylphenyl]amino}prop-1- |
| | yn-1-yl)-1-(2,2,2-trifluoroethyl)-1H-indol-4-yl]amino}piperidin-1-yl)-3- |
| | methoxypropan-2-ol |
| 1805. | 1-(4-{[2-(3-{[2-(difluoromethoxy)-4-methanesulfonylphenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1806. | 3-(2-hydroxy-3-{4-[(2-{3-[(4-methanesulfonyl-2-methoxyphenyl)amino]prop-1- |
| | yn-1-yl}-1-(2,2,2-trifluoroethyl)-1H-indol-4-yl)amino]piperidin-1- |
| | yl}propoxy)propane-1,2-diol |
| 1807. | 1-{4-[(2-{3-[(5-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1808. | 3-(3-(4-((1-(2-hydroxy-3-methoxypropyl)piperidin-4-yl)amino)-1-(2,2,2- |
| | trifluoroethyl)-1H-indol-2-yl)prop-2-yn-1-yl)-6-(methylsulfonyl)benzo[d]oxazol- |
| | 2(3H)-one |
| 1809. | 1-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1810. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-(2,2,2- |
| | trifluoroethoxy)propan-2-ol |
| 1811. | 4-hydroxy-9-[(2-{3-[(4-methanesulfonyl-2-methoxyphenyl)amino]prop-1-yn-1- |
| | yl}-1-(2,2,2-trifluoroethyl)-1H-indol-4-yl)amino]-2-oxa-6λ⁵- |
| | azaspiro[5.5]undecan-6-ylium |
| 1812. | 2-{3-[(4-methanesulfonyl-2-methoxyphenyl)amino]prop-1-yn-1-yl}-N-[1-(3- |
| | methoxypropyl)piperidin-4-yl]-1-(2,2,2-trifluoroethyl)-1H-indol-4-amine |
| 1813. | 1-{4-[(2-{3-[(4-methanesulfonyl-2-methoxyphenyl)amino]prop-1-yn-1-yl}-3- |
| | methyl-1-(2,2,2-trifluoroethyl)-1H-indol-4-yl)amino]piperidin-1-yl}-3- |
| | methoxypropan-2-ol |
| 1814. | 1-{4-[(2-{3-[(5-fluoro-4-methanesulfonyl-2-methoxyphenyl)amino]prop-1-yn-1- |
| | yl}-1-(2,2,2-trifluoroethyl)-1H-indol-4-yl)amino]piperidin-1-yl}-3- |
| | methoxypropan-2-ol |
| 1815. | 2-{3-[(4-methanesulfonyl-2-methoxyphenyl)amino]prop-1-yn-1-yl}-N-[1-(oxetan- |
| | 3-yl)piperidin-4-yl]-1-(2,2,2-trifluoroethyl)-1H-indol-4-amine |
| 1816. | 2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1817. | (R)-2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1818. | (S)-2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1819. | 2-(3-((2-methoxy-4-(methylsulfonyl)phenyl)amino)prop-1-yn-1-yl)-N-(1- |
| | ((tetrahydrofuran-2-yl)methyl)piperidin-4-yl)-1-(2,2,2-trifluoroethyl)-1H-indol-4- |
| | amine |
| 1820. | 2-fluoro-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-5-methoxybenzamide |
| 1821. | 1-methoxy-3-(4-((2-(3-(((3S,4R)-3-methoxytetrahydro-2H-pyran-4- |
| | yl)amino)prop-1-yn-1-yl)-1-(2,2,2-trifluoroethyl)-1H-indol-4-yl)amino)piperidin- |
| | 1-yl)propan-2-ol |
| 1822. | 1-(4-{[2-(3-{[4-(cyclopropanesulfonyl)-2-methoxyphenyl]amino}prop-1-yn-1-yl)- |
| | 1-(2,2,2-trifluoroethyl)-1H-indol-4-yl]amino}piperidin-1-yl)-3-methoxypropan-2- |
| | ol |
| 1823. | 3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propanenitrile |
| 1824. | 4-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)butanenitrile |
| 1825. | 2-{3-[(4-methanesulfonyl-2-methoxyphenyl)amino]prop-1-yn-1-yl}-N-{1- |
| | [(oxolan-2-yl)methyl]piperidin-4-yl}-1-(2,2,2-trifluoroethyl)-1H-indol-4-amine |
| 1826. | 1-methoxy-3-(4-{[2-(3-{[2-methoxy-4-(trifluoromethyl)phenyl]amino}prop-1-yn- |
| | 1-yl)-1-(2,2,2-trifluoroethyl)-1H-indol-4-yl]amino}piperidin-1-yl)propan-2-ol |
| 1827. | 2-fluoro-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-5-methoxy-N- |
| | methylbenzamide |
| 1828. | 1-(4-{[2-(3-{[4-(benzenesulfonyl)-2-methoxyphenyl]amino}prop-1-yn-1-yl)-1- |
| | (2,2,2-trifluoroethyl)-1H-indol-4-yl]amino}piperidin-1-yl)-3-methoxypropan-2-ol |
| 1829. | 2-{3-[(4-methanesulfonyl-2-methoxyphenyl)amino]prop-1-yn-1-yl}-N-[1-(2- |
| | methoxy-2-methylpropyl)piperidin-4-yl]-1-(2,2,2-trifluoroethyl)-1H-indol-4- |
| | amine |
| 1830. | 2-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)ethan-1-ol |
| 1831. | 1-methoxy-3-(4-{[2-(3-{[2-methoxy-4-(propane-2-sulfonyl)phenyl]amino}prop-1- |
| | yn-1-yl)-1-(2,2,2-trifluoroethyl)-1H-indol-4-yl]amino}piperidin-1-yl)propan-2-ol |
| 1832. | 1-{3-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1833. | 1-methoxy-3-(4-((2-(3-(((3R,4R)-3-methoxytetrahydro-2H-pyran-4- |
| | yl)amino)prop-1-yn-1-yl)-1-(2,2,2-trifluoroethyl)-1H-indol-4-yl)amino)piperidin- |
| | 1-yl)propan-2-ol |
| 1834. | 1-{4-[(2-{3-[(4-methanesulfonyl-2-methoxyphenyl)amino]prop-1-yn-1-yl}-1- |
| | propyl-1H-indol-4-yl)amino]piperidin-1-yl}-3-methoxypropan-2-ol |
| 1835. | 1-(4-{[1-(2-fluoroethyl)-2-{3-[(4-methanesulfonyl-2-methoxyphenyl)amino]prop- |
| | 1-yn-1-yl}-1H-indol-4-yl]amino}piperidin-1-yl)-3-methoxypropan-2-ol |
| 1836. | 2-(4-{[1-(2-hydroxy-3-methoxypropyl)piperidin-4-yl]amino}-2-{3-[(4- |
| | methanesulfonyl-2-methoxyphenyl)amino]prop-1-yn-1-yl}-1H-indol-1- |
| | yl)acetonitrile |
| 1837. | 1-(4-{[1-(2-chloroethyl)-2-{3-[(4-methanesulfonyl-2-methoxyphenyl)amino]prop- |
| | 1-yn-1-yl}-1H-indol-4-yl]amino}piperidin-1-yl)-3-methoxypropan-2-ol |
| 1838. | rac-1-[(3R,4S)-4-[(2-{3-[(4-methanesulfonyl-2-methoxyphenyl)amino]prop-1-yn- |
| | 1-yl}-1-(2,2,2-trifluoroethyl)-1H-indol-4-yl)amino]-3-methylpiperidin-1-yl]-3- |
| | methoxypropan-2-ol |
| 1839. | rac-1-[(3R,4R)-4-[(2-{3-[(4-methanesulfonyl-2-methoxyphenyl)amino]prop-1-yn- |
| | 1-yl}-1-(2,2,2-trifluoroethyl)-1H-indol-4-yl)amino]-3-methylpiperidin-1-yl]-3- |
| | methoxypropan-2-ol |
| 1840. | 1-{4-[(2-{3-[(4-methanesulfonyl-2-methylphenyl)amino]prop-1-yn-1-yl}-1-(2,2,2- |
| | trifluoroethyl)-1H-indol-4-yl)amino]piperidin-1-yl}-3-methoxypropan-2-ol |
| 1841. | 1-[4-({1-[(2,2-difluorocyclopropyl)methyl]-2-{3-[(4-methanesulfonyl-2- |
| | methoxyphenyl)amino]prop-1-yn-1-yl}-1H-indol-4-yl}amino)piperidin-1-yl]-3- |
| | methoxypropan-2-ol |
| 1842. | 4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzoic acid |
| 1843. | 2-{3-[(4-methanesulfonyl-2-methoxyphenyl)amino]prop-1-yn-1-yl}-N-[1-(3- |
| | methanesulfonylpropyl)piperidin-4-yl]-1-(2,2,2-trifluoroethyl)-1H-indol-4-amine |
| 1844. | 4-{[3-(4-{[1-(3-methanesulfonylpropyl)piperidin-4-yl]amino}-1-(2,2,2- |
| | trifluoroethyl)-1H-indol-2-yl)prop-2-yn-1-yl]amino}-3-methoxybenzoic acid |
| 1845. | 1-(4-{[2-(3-{[4-methanesulfonyl-2-(trifluoromethyl)phenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1846. | 1-(4-{[1-(2,2-difluoroethyl)-2-{3-[(4-methanesulfonyl-2- |
| | methoxyphenyl)ino]prop-1-yn-1-yl}-1H-indol-4-yl]amino}piperidin-1-yl)-3- |
| | methoxypropan-2-ol |
| 1847. | 1-(4-{[2-(3-{[4-methanesulfonyl-2-(trifluoromethoxy)phenyl]amino}prop-1-yn-1- |
| | yl)-1-(2,2,2-trifluoroethyl)-1H-indol-4-yl]amino}piperidin-1-yl)-3- |
| | methoxypropan-2-ol |
| 1848. | 1-{4-[(2-{3-[(4-methanesulfonyl-2-methoxyphenyl)amino]prop-1-yn-1-yl}-1- |
| | (3,3,3-trifluoropropyl)-1H-indol-4-yl)amino]piperidin-1-yl}-3-methoxypropan-2- |
| | ol |
| 1849. | 1-(4-{[1-(2,2-difluoropropyl)-2-{3-[(4-methanesulfonyl-2- |
| | methoxyphenyl)amino]prop-1-yn-1-yl}-1H-indol-4-yl]amino}piperidin-1-yl)-3- |
| | methoxypropan-2-ol |
| 1850. | 1-{4-[(2-{3-[(2-chloro-4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1851. | 1-{4-[(2-{3-[(2-fluoro-4-methanesulfonyl-6-methoxyphenyl)amino]prop-1-yn-1- |
| | yl}-1-(2,2,2-trifluoroethyl)-1H-indol-4-yl)amino]piperidin-1-yl}-3- |
| | methoxypropan-2-ol |
| 1852. | 1-(4-((2-(3-((4-((6-oxa-3-azabicyclo[3.1.1]heptan-3-yl)sulfonyl)-2- |
| | methoxyphenyl)amino)prop-1-yn-1-yl)-1-(2,2,2-trifluoroethyl)-1H-indol-4- |
| | yl)amino)piperidin-1-yl)-3-methoxypropan-2-ol |
| 1853. | (2S)-1-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}-3-methoxypropan-2- |
| | yl propanoate |
| 1854. | 1-{4-[(2-{3-[(4-chloro-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1855. | 2-(2-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)ethoxy)ethan-1-ol |
| 1856. | 4-((3-(4-((1-(2,3-dihydroxypropyl)piperidin-4-yl)amino)-1-(2,2,2-trifluoroethyl)- |
| | 1H-indol-2-yl)prop-2-yn-1-yl)amino)-N-(2-hydroxyethyl)-3- |
| | methoxybenzenesulfonamide |
| 1857. | 2-{3-[(4-methanesulfonyl-2-methoxyphenyl)amino]prop-1-yn-1-yl}-N-[1-(4- |
| | methyl-1,3-thiazol-2-yl)piperidin-4-yl]-1-(2,2,2-trifluoroethyl)-1H-indol-4-amine |
| 1858. | N-(1-cyclopropylpiperidin-4-yl)-2-{3-[(4-methanesulfonyl-2- |
| | methoxyphenyl)amino]prop-1-yn-1-yl}-1-(2,2,2-trifluoroethyl)-1H-indol-4-amine |
| 1859. | 4-[(3-{4-[(1-cyclopropylpiperidin-4-yl)amino]-1-(2,2,2-trifluoroethyl)-1H-indol- |
| | 2-yl}prop-2-yn-1-yl)amino]-3-methoxybenzoic acid |
| 1860. | 2-{3-[(4-methanesulfonyl-2-methoxyphenyl)amino]prop-1-yn-1-yl}-N-{1-[(3R)- |
| | oxolan-3-yl]piperidin-4-yl}-1-(2,2,2-trifluoroethyl)-1H-indol-4-amine |
| 1861. | 2-{3-[(4-methanesulfonyl-2-methoxyphenyl)amino]prop-1-yn-1-yl}-N-{1-[(3S)- |
| | oxolan-3-yl]piperidin-4-yl}-1-(2,2,2-trifluoroethyl)-1H-indol-4-amine |
| 1862. | 3-methoxy-4-{[3-(4-{[1-(oxan-4-yl)piperidin-4-yl]amino}-1-(2,2,2-trifluoroethyl)- |
| | 1H-indol-2-yl)prop-2-yn-1-yl]amino}benzoic acid |
| 1863. | 3-methoxy-4-((3-(4-((1-(tetrahydro-2H-pyran-4-yl)piperidin-4-yl)amino)-1-(2,2,2- |
| | trifluoroethyl)-1H-indol-2-yl)prop-2-yn-1-yl)amino)benzamide |
| 1864. | N-(2-hydroxyethyl)-3-methoxy-4-((3-(4-((1-(tetrahydro-2H-pyran-4-yl)piperidin- |
| | 4-yl)amino)-1-(2,2,2-trifluoroethyl)-1H-indol-2-yl)prop-2-yn-1- |
| | yl)amino)benzenesulfonamide |
| 1865. | 3-methoxy-4-((3-(4-((1′-methyl-[1,4′-bipiperidin]-4-yl)amino)-1-(2,2,2- |
| | trifluoroethyl)-1H-indol-2-yl)prop-2-yn-1-yl)amino)benzamide |
| 1866. | 3-methoxy-4-((3-(4-((1-(2-(4-methylpiperazin-1-yl)-2-oxoethyl)piperidin-4- |
| | yl)amino)-1-(2,2,2-trifluoroethyl)-1H-indol-2-yl)prop-2-yn-1-yl)amino)benzamide |
| 1867. | 2-{2-[(3-{4-[(1-acetylpiperidin-4-yl)amino]-1-(2,2,2-trifluoroethyl)-1H-indol-2- |
| | yl}prop-2-yn-1-yl)amino]-5-methanesulfonylphenoxy}acetonitrile |
| 1868. | 4-{[3-(4-{[1-(2-hydroxyacetyl)piperidin-4-yl]amino}-1-(2,2,2-trifluoroethyl)-1H- |
| | indol-2-yl)prop-2-yn-1-yl]amino}-3-methoxy-N,N-dimethylbenzene-1- |
| | sulfonamide |
| 1869. | 2-hydroxy-1-{4-[(2-{3-[(2-hydroxy-4-methanesulfonylphenyl)amino]prop-1-yn-1- |
| | yl}-1-(2,2,2-trifluoroethyl)-1H-indol-4-yl)amino]piperidin-1-yl}ethan-1-one |
| 1870. | 2-hydroxy-1-{4-[(2-{3-[(4-methanesulfonyl-2-methoxyphenyl)amino]prop-1-yn- |
| | 1-yl}-1-(2,2,2-trifluoroethyl)-1H-indol-4-yl)amino]piperidin-1-yl}ethan-1-one |
| 1871. | N-((3S,4S)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1872. | N-((3R,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1873. | N-((3R,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1874. | N-((3R,4S)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1875. | N-((3S,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1876. | 1-(4-((2-(3-((2-methoxy-4-(methylsulfonyl)phenyl)amino)prop-1-yn-1-yl)-1- |
| | (2,2,2-trifluoroethyl)-1H-indol-4-yl)amino)-2-methylpiperidin-1-yl)ethan-1-one |
| 1877. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]-2-methylpiperidin-1-yl}ethan-1-one |
| 1878. | 4-{[3-(4-{[(2S,4S)-1-acetyl-2-methylpiperidin-4-yl]amino}-1-(2,2,2- |
| | trifluoroethyl)-1H-indol-2-yl)prop-2-yn-1-yl]amino}-3-methoxybenzene-1- |
| | sulfonamide |
| 1879. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-2-methoxyethan-1-one |
| 1880. | 2-hydroxy-1-{4-[(2-{3-[(4-methanesulfonyl-2-methoxyphenyl)amino]prop-1-yn- |
| | 1-yl}-1-(2,2,2-trifluoroethyl)-1H-indol-4-yl)amino]piperidin-1-yl}propan-1-one |
| 1881. | 2-methoxy-1-(4-((2-(3-((2-methoxy-4-(methylsulfonyl)phenyl)amino)prop-1-yn- |
| | 1-yl)-1-(2,2,2-trifluoroethyl)-1H-indol-4-yl)amino)piperidin-1-yl)ethan-1-one |
| 1882. | 3-methoxy-4-((3-(4-((1-(2-methoxyacetyl)piperidin-4-yl)amino)-1-(2,2,2- |
| | trifluoroethyl)-1H-indol-2-yl)prop-2-yn-1-yl)amino)benzenesulfonamide |
| 1883. | 4-{[3-(4-{[1-(2-hydroxypropanoyl)piperidin-4-yl]amino}-1-(2,2,2-trifluoroethyl)- |
| | 1H-indol-2-yl)prop-2-yn-1-yl]amino}-3-methoxybenzene-1-sulfonamide |
| 1884. | 3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-3-oxopropanenitrile |
| 1885. | 4-{[3-(4-{[1-(2-cyanoacetyl)piperidin-4-yl]amino}-1-(2,2,2-trifluoroethyl)-1H- |
| | indol-2-yl)prop-2-yn-1-yl]amino}-3-methoxybenzene-1-sulfonamide |
| 1886. | 4-{[3-(4-{[1-(2-hydroxyacetyl)piperidin-4-yl]amino}-1-(2,2,2-trifluoroethyl)-1H- |
| | indol-2-yl)prop-2-yn-1-yl]amino}-3-methoxybenzene-1-sulfonamide |
| 1887. | 4-[(3-{4-[(1-acetylpiperidin-4-yl)amino]-1-(2,2,2-trifluoroethyl)-1H-indol-2- |
| | yl}prop-2-yn-1-yl)amino]-3-methoxybenzene-1-sulfonamide |
| 1888. | 2-(dimethylamino)-1-{4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl}ethan-1-one |
| 1889. | 4-((3-(4-((1-(dimethylglycyl)piperidin-4-yl)amino)-1-(2,2,2-trifluoroethyl)-1H- |
| | indol-2-yl)prop-2-yn-1-yl)amino)-3-methoxybenzenesulfonamide |
| 1890. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}-2-methylpropan-1-one |
| 1891. | 3-methoxy-4-{[3-(4-{[1-(2-methylpropanoyl)piperidin-4-yl]amino}-1-(2,2,2- |
| | trifluoroethyl)-1H-indol-2-yl)prop-2-yn-1-yl]amino}benzene-1-sulfonamide |
| 1892. | 4-((2-(3-((2-methoxy-4-(methylsulfonyl)phenyl)amino)prop-1-yn-1-yl)-1-(2,2,2- |
| | trifluoroethyl)-1H-indol-4-yl)amino)-N,N-dimethylpiperidine-1-carboxamide |
| 1893. | 4-[(3-{4-[(1-acetylpiperidin-4-yl)amino]-1-(2,2,2-trifluoroethyl)-1H-indol-2- |
| | yl}prop-2-yn-1-yl)amino]-3-methoxy-N,N-dimethylbenzene-1-sulfonamide |
| 1894. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-1-one |
| 1895. | 3-methoxy-4-[(3-{4-[(1-propanoylpiperidin-4-yl)amino]-1-(2,2,2-trifluoroethyl)- |
| | 1H-indol-2-yl}prop-2-yn-1-yl)amino]benzene-1-sulfonamide |
| 1896. | 1-(4-{[2-(3-{[2-methoxy-4-(morpholine-4-sulfonyl)phenyl]amino}prop-1-yn-1- |
| | yl)-1-(2,2,2-trifluoroethyl)-1H-indol-4-yl]amino}piperidin-1-yl)ethan-1-one |
| 1897. | 3-methoxy-4-[(3-{4-[(1-{[(4S)-2-oxo-1,3-dioxolan-4-yl]methyl}piperidin-4- |
| | yl)amino]-1-(2,2,2-trifluoroethyl)-1H-indol-2-yl}prop-2-yn-1-yl)amino]benzene- |
| | 1-sulfonamide |
| 1898. | N-((3-methoxy-4-((3-(4-((1-((2-oxo-1,3-dioxolan-4-yl)methyl)piperidin-4- |
| | yl)amino)-1-(2,2,2-trifluoroethyl)-1H-indol-2-yl)prop-2-yn-1- |
| | yl)amino)phenyl)sulfonyl)propionamide |
| 1899. | N-[3-methoxy-4-({3-[4-({1-[(2-oxo-1,3-dioxolan-4-yl)methyl]piperidin-4- |
| | yl}amino)-1-(2,2,2-trifluoroethyl)-1H-indol-2-yl]prop-2-yn-1- |
| | yl}amino)benzenesulfonyl]acetamide |
| 1900. | 3-methoxy-N-methyl-4-({3-[4-({1-[(2-oxo-1,3-dioxolan-4-yl)methyl]piperidin-4- |
| | yl}amino)-1-(2,2,2-trifluoroethyl)-1H-indol-2-yl]prop-2-yn-1- |
| | yl}amino)benzamide |
| 1901. | 2-(3-((2-methoxy-4-(methylsulfonyl)phenyl)amino)prop-1-yn-1-yl)-N- |
| | (octahydroindolizin-7-yl)-1-(2,2,2-trifluoroethyl)-1H-indol-4-amine |
| 1902. | N-[(7R,8aS)-octahydroindolizin-7-yl]-2-{3-[(4-methanesulfonyl-2- |
| | methoxyphfenyl)amino]prop-1-yn-1-yl}-1-(2,2,2-trifluoroethyl)-1H-indol-4-amine |
| 1903. | N-[(7R,8aR)-octahydroindolizin-7-yl]-2-{3-[(4-methanesulfonyl-2- |
| | methoxyphenyl)amino]prop-1-yn-1-yl}-1-(2,2,2-trifluoroethyl)-1H-indol-4-amine |
| 1904. | rac-(3R,4S)-3-[(2-{3-[(4-methanesulfonyl-2-methoxyphenyl)amino]prop-1-yn-1- |
| | yl}-1-(2,2,2-trifluoroethyl)-1H-indol-4-yl)amino]-1-methylpiperidin-4-ol |
| 1905. | rac-(3R,4R)-4-[(2-{3-[(4-methanesulfonyl-2-methoxyphenyl)amino]prop-1-yn-1- |
| | yl}-1-(2,2,2-trifluoroethyl)-1H-indol-4-yl)amino]-1-methylpiperidin-3-ol |
| 1906. | 3-methoxy-4-((3-(4-((1-(tetrahydro-2H-pyran-4-yl)piperidin-4-yl)amino)-1-(2,2,2- |
| | trifluoroethyl)-1H-indol-2-yl)prop-2-yn-1-yl)amino)benzenesulfonamide |
| 1907. | rac-N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1908. | N-((3S,4S)-3-fluoro-1-methylpiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1909. | N-[(3S,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1910. | N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1911. | N-[(3S,4R)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1912. | N-[(3R,4R)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1913. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzene-1- |
| | sulfonamide |
| 1914. | rac-4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzene-1- |
| | sulfonamide |
| 1915. | rac-methyl 4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1916. | rac-methyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1917. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoic acid |
| 1918. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoic acid |
| 1919. | 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoic acid |
| 1920. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 1921. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxy-N- |
| | methylbenzamide |
| 1922. | 4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxy-N- |
| | methylbenzamide |
| 1923. | rac-4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N- |
| | methylbenzamide |
| 1924. | 2-fluoro-4-((3-(4-(((3R,4S)-3-fluoro-1-methylpiperidin-4-yl)amino)-1-(2,2,2- |
| | trifluoroethyl)-1H-indol-2-yl)prop-2-yn-1-yl)amino)-5-methoxy-N- |
| | methylbenzamide |
| 1925. | 2-fluoro-4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-5-methoxy-N- |
| | methylbenzamide |
| 1926. | 4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoic acid |
| 1927. | 4-{[3-(4-{[(3S,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoic acid |
| 1928. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzene-1- |
| | sulfonamide |
| 1929. | 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzene-1- |
| | sulfonamide |
| 1930. | methyl 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1931. | rac-4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 1932. | methyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1933. | rac-N-[(3R,4S)-3-fluoro-1-(propan-2-yl)piperidin-4-yl]-2-{3-[(4-methanesulfonyl- |
| | 2-methoxyphenyl)amino]prop-1-yn-1-yl}-1-(2,2,2-trifluoroethyl)-1H-indol-4- |
| | amine |
| 1934. | rac-4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1935. | rac-ethyl 4-{[3-(4-{[(3R,4S)-3-fluoro-1-(propan-2-yl)piperidin-4-yl]amino}-1- |
| | (2,2,2-trifluoroethyl)-1H-indol-2-yl)prop-2-yn-1-yl]amino}-3-methoxybenzoate |
| 1936. | rac-ethyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1937. | (2R)-1-(acetyloxy)-3-[(3RS,4SR)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 1938. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 1939. | 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 1940. | N-[(3S,4R)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1941. | N-[(3R,4S)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1942. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N- |
| | methylbenzamide |
| 1943. | 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxy-N- |
| | methylbenzamide |
| 1944. | rac-2-hydroxypropyl 4-{[3-(4-{[(3R,4S)-3-fluoro-1-methylpiperidin-4-yl]amino}- |
| | 1-(2,2,2-trifluoroethyl)-1H-indol-2-yl)prop-2-yn-1-yl]amino}-3-methoxybenzoate |
| 1945. | 4-((3-(4-(((3R,4S)-3-fluoro-1-methylpiperidin-4-yl)amino)-1-(2,2,2- |
| | trifluoroethyl)-1H-indol-2-yl)prop-2-yn-1-yl)amino)-N-isopropyl-3- |
| | methoxybenzamide |
| 1946. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-(propan-2- |
| | yl)benzamide |
| 1947. | 4-{[3-(4-{[(3S,4R)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1948. | 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1949. | rac-2-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]acetamide |
| 1950. | N-[(3S,4R)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1951. | N-[(3R,4S)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1952. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxy-N-(2- |
| | methoxyethyl)benzamide |
| 1953. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxy-N,N- |
| | dimethylbenzamide |
| 1954. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-N-(2-hydroxy-3- |
| | methoxypropyl)-3-methoxybenzamide |
| 1955. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxy-N-(propan-2- |
| | yl)benzamide |
| 1956. | rac-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxy-N-(oxan-4- |
| | yl)benzamide |
| 1957. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-(oxan-4- |
| | yl)benzamide |
| 1958. | rac-N-[(3R,4S)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1959. | ethyl 4-{[3-(4-{[(3S,4R)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1960. | ethyl 4-((3-(4-(((3R,4S)-1-ethyl-3-fluoropiperidin-4-yl)amino)-1-(2,2,2- |
| | trifluoroethyl)-1H-indol-2-yl)prop-2-yn-1-yl)amino)-3-methoxybenzoate |
| 1961. | ethyl 4-{[3-(4-{[(3S,4R)-1-ethyl-3-fluoropiperidin-4-yl]amino}-1-(2,2,2- |
| | trifluoroethyl)-1H-indol-2-yl)prop-2-yn-1-yl]amino}-3-methoxybenzoate |
| 1962. | ethyl 4-{[3-(4-{[(3R,4S)-1-ethyl-3-fluoropiperidin-4-yl]amino}-1-(2,2,2- |
| | trifluoroethyl)-1H-indol-2-yl)prop-2-yn-1-yl]amino}-3-methoxybenzoate |
| 1963. | ethyl 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1964. | 2-fluoro-4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-5-methoxy-N- |
| | methylbenzamide |
| 1965. | 2-fluoro-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-5-methoxy-N- |
| | methylbenzamide |
| 1966. | 4-{[3-(4-{[(3R,4S)-1-ethyl-3-fluoropiperidin-4-yl]amino}-1-(2,2,2-trifluoroethyl)- |
| | 1H-indol-2-yl)prop-2-yn-1-yl]amino}-3-methoxybenzoic acid |
| 1967. | 4-{[3-(4-{[(3S,4R)-1-ethyl-3-fluoropiperidin-4-yl]amino}-1-(2,2,2-trifluoroethyl)- |
| | 1H-indol-2-yl)prop-2-yn-1-yl]amino}-3-methoxybenzoic acid |
| 1968. | rac-N-(2-{bis[(pyridin-2-yl)methyl]amino}ethyl)-4-{[3-(4-{[(3R,4S)-3-fluoro-1- |
| | methylpiperidin-4-yl]amino}-1-(2,2,2-trifluoroethyl)-1H-indol-2-yl)prop-2-yn-1- |
| | yl]amino}-3-methoxybenzamide |
| 1969. | rac-N-[(3R,4S)-3-fluoro-1-methylpiperidin-4-yl]-2-{3-[(4-methanesulfonyl-2- |
| | methoxyphenyl)amino]prop-1-yn-1-yl}-N-methyl-1-(2,2,2-trifluoroethyl)-1H- |
| | indol-4-amine |
| 1970. | rac-4-((3-(4-(((3R,4S)-3-fluoro-1-methylpiperidin-4-yl)amino)-1-(2,2,2- |
| | trifluoroethyl)-1H-indol-2-yl)prop-2-yn-1-yl)amino)-3-methoxybenzoic acid |
| 1971. | rac-4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoic acid |
| 1972. | 2-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl] amino}-5-methanesulfonylphenol |
| 1973. | 2-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl] amino}-5-methanesulfonylphenol |
| 1974. | rac-6-fluoro-N-[(3R,4S)-3-fluoro-1-methylpiperidin-4-yl]-2-{3-[(4- |
| | methanesulfonyl-2-methoxyphenyl)amino]prop-1-yn-1-yl}-1-(2,2,2- |
| | trifluoroethyl)-1H-indol-4-amine |
| 1975. | N-(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl] amino}-3- |
| | methoxybenzenesulfonyl)acetamide |
| 1976. | N-(4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl] amino}-3- |
| | methoxybenzenesulfonyl)acetamide |
| 1977. | ethyl 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl] amino}-3-methoxybenzoate |
| 1978. | ethyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl] amino}-3-methoxybenzoate |
| 1979. | N-[(3R,4S)-3-fluoro-1-methylpiperidin-4-yl]-2-(3-{[2-(fluoromethoxy)-4- |
| | methanesulfonylphenyl]amino}prop-1-yn-1-yl)-1-(2,2,2-trifluoroethyl)-1H-indol- |
| | 4-amine |
| 1980. | N-[(3S,4R)-3-fluoro-1-methylpiperidin-4-yl]-2-(3-{[2-(fluoromethoxy)-4- |
| | methanesulfonylphenyl]amino}prop-1-yn-1-yl)-1-(2,2,2-trifluoroethyl)-1H-indol- |
| | 4-amine |
| 1981. | 2-{3-[(2-ethoxy-4-methanesulfonylphenyl)amino]prop-1-yn-1-yl}-N-[(3S,4R)-3- |
| | fluoro-1-methylpiperidin-4-yl]-1-(2,2,2-trifluoroethyl)-1H-indol-4-amine |
| 1982. | 2-{3-[(2-ethoxy-4-methanesulfonylphenyl)amino]prop-1-yn-1-yl}-N-[(3R,4S)-3- |
| | fluoro-1-methylpiperidin-4-yl]-1-(2,2,2-trifluoroethyl)-1H-indol-4-amine |
| 1983. | 4-{[3-(4-{[(3S,4R)-3-fluoropiperidin-4-yl]amino}-1-(2,2,2-trifluoroethyl)-1H- |
| | indol-2-yl)prop-2-yn-1-yl]amino}-3-methoxybenzene-1-sulfonamide |
| 1984. | 4-{[3-(4-{[(3R,4S)-3-fluoropiperidin-4-yl]amino}-1-(2,2,2-trifluoroethyl)-1H- |
| | indol-2-yl)prop-2-yn-1-yl]amino}-3-methoxybenzene-1-sulfonamide |
| 1985. | N-((3S,4R)-3-fluoropiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1986. | N-((3R,4S)-3-fluoropiperidin-4-yl)-2-(3-((2-methoxy-4- |
| | (methylsulfonyl)phenyl)amino)prop-1-yn-1-yl)-1-(2,2,2-trifluoroethyl)-1H-indol- |
| | 4-amine |
| 1987. | 4-{[3-(4-{[(3S,4R)-3-fluoropiperidin-4-yl]amino}-1-(2,2,2-trifluoroethyl)-1H- |
| | indol-2-yl)prop-2-yn-1-yl]amino}-3-methoxybenzamide |
| 1988. | 4-((3-(4-(((3R,4S)-3-fluoropiperidin-4-yl)amino)-1-(2,2,2-trifluoroethyl)-1H- |
| | indol-2-yl)prop-2-yn-1-yl)amino)-3-methoxybenzamide |
| 1989. | 4-{[3-(4-{[(3R,4S)-3-fluoropiperidin-4-yl]amino}-1-(2,2,2-trifluoroethyl)-1H- |
| | indol-2-yl)prop-2-yn-1-yl]amino}-3-methoxy-N-methylbenzamide |
| 1990. | 4-{[3-(4-{[(3R,4S)-3-fluoropiperidin-4-yl]amino}-1-(2,2,2-trifluoroethyl)-1H- |
| | indol-2-yl)prop-2-yn-1-yl]amino}-3-methoxybenzoic acid |
| 1991. | 2-[(3S,4R)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]ethan-1-ol |
| 1992. | 2-[(3R,4S)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]ethan-1-ol |
| 1993. | 2-(dimethylamino)-1-[(3S,4R)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 1994. | 2-(dimethylamino)-1-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 1995. | 4-{[3-(4-{[(3S,4R)-3-fluoropiperidin-4-yl]amino}-1-(2,2,2-trifluoroethyl)-1H- |
| | indol-2-yl)prop-2-yn-1-yl]amino}-3-methoxy-N-methylbenzamide |
| 1996. | 4-{[3-(4-{[(3S,4R)-3-fluoropiperidin-4-yl]amino}-1-(2,2,2-trifluoroethyl)-1H- |
| | indol-2-yl)prop-2-yn-1-yl]amino}-3-methoxybenzoic acid |
| 1997. | N-[(3S,4R)-3-fluoro-1-(2-methoxyethyl)piperidin-4-yl]-2-{3-[(4-methanesulfonyl- |
| | 2-methoxyphenyl)amino]prop-1-yn-1-yl}-1-(2,2,2-trifluoroethyl)-1H-indol-4- |
| | amine |
| 1998. | N-[(3R,4S)-3-fluoro-1-(2-methoxyethyl)piperidin-4-yl]-2-{3-[(4-methanesulfonyl- |
| | 2-methoxyphenyl)amino]prop-1-yn-1-yl}-1-(2,2,2-trifluoroethyl)-1H-indol-4- |
| | amine |
| 1999. | 4-{[3-(4-{[(3S,4R)-3-fluoro-1-[(2R)-2-hydroxy-3-methoxypropyl]piperidin-4- |
| | yl]amino}-1-(2,2,2-trifluoroethyl)-1H-indol-2-yl)prop-2-yn-1-yl]amino}-3- |
| | methoxybenzene-1-sulfonamide |
| 2000. | 1-[(3S,4R)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]ethan-1- |
| | one |
| 2001. | 1-[(3R,4S)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]ethan-1- |
| | one |
| 2002. | 1-[(3S,4R)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]propan-1- |
| | one |
| 2003. | 1-[(3R,4S)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]propan-1- |
| | one |
| 2004. | 1-[(3S,4R)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]-2- |
| | methoxyethan-1-one |
| 2005. | 1-[(3R,4S)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]-2- |
| | methoxyethan-1-one |
| 2006. | 4-{[3-(4-{[(3R,4S)-3-fluoropiperidin-4-yl]amino}-1-(2,2,2-trifluoroethyl)-1H- |
| | indol-2-yl)prop-2-yn-1-yl]amino}-3-methoxybenzamide |
| 2007. | 4-{[3-(4-{[(3R,4S)-3-fluoro-1-[(2R)-2-hydroxy-3-methoxypropyl]piperidin-4- |
| | yl]amino}-1-(2,2,2-trifluoroethyl)-1H-indol-2-yl)prop-2-yn-1-yl]amino}-3- |
| | methoxybenzene-1-sulfonamide |
| 2008. | N-[(7S,8R)-7-fluoro-1,4-dioxaspiro[4.5]decan-8-yl]-2-{3-[(4-methanesulfonyl-2- |
| | methoxyphenyl)amino]prop-1-yn-1-yl}-1-(2,2,2-trifluoroethyl)-1H-indol-4-amine |
| 2009. | 1-[(3S,4R)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]propan-2- |
| | ol |
| 2010. | 1-((3S,4S)-3-fluoro-4-((2-(3-((2-methoxy-4-(methylsulfonyl)phenyl)amino)prop- |
| | 1-yn-1-yl)-1-(2,2,2-trifluoroethyl)-1H-indol-4-yl)amino)piperidin-1-yl)-3- |
| | methoxypropan-2-ol |
| 2011. | 1-((3R,4S)-3-fluoro-4-((2-(3-((2-methoxy-4-(methylsulfonyl)phenyl)amino)prop- |
| | 1-yn-1-yl)-1-(2,2,2-trifluoroethyl)-1H-indol-4-yl)amino)piperidin-1-yl)-3- |
| | methoxypropan-2-ol |
| 2012. | 4-((3-(4-(((3S,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2013. | 4-((3-(4-(((3R,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2014. | 4-((3-(4-(((3R,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2015. | 4-((3-(4-(((3S,4R)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2016. | methyl 4-((3-(4-(((3S,4R)-3-fluoro-1-((R)-2-hydroxy-3-methoxypropyl)piperidin- |
| | 4-yl)amino)-1-(2,2,2-trifluoroethyl)-1H-indol-2-yl)prop-2-yn-1-yl)amino)-3- |
| | methoxybenzoate |
| 2017. | methyl 4-((3-(4-(((3R,4S)-3-fluoro-1-((R)-2-hydroxy-3-methoxypropyl)piperidin- |
| | 4-yl)amino)-1-(2,2,2-trifluoroethyl)-1H-indol-2-yl)prop-2-yn-1-yl)amino)-3- |
| | methoxybenzoate |
| 2018. | (R)-1-((3R,4S)-3-fluoro-4-((2-(3-((2-fluoro-6-methoxy-4- |
| | (methylsulfonyl)phenyl)amino)prop-1-yn-1-yl)-1-propyl-1H-indol-4- |
| | yl)amino)piperidin-1-yl)-3-methoxypropan-2-ol |
| 2019. | (R)-1-((3R,4S)-4-((1-allyl-2-(3-((2-fluoro-6-methoxy-4- |
| | (methylsulfonyl)phenyl)amino)prop-1-yn-1-yl)-1H-indol-4-yl)amino)-3- |
| | fluoropiperidin-1-yl)-3-methoxypropan-2-ol |
| 2020. | 4-{[3-(4-{[(3S,4R)-1-[(2R)-2,3-dihydroxypropyl]-3-fluoropiperidin-4-yl]amino}- |
| | 1-(2,2,2-trifluoroethyl)-1H-indol-2-yl)prop-2-yn-1-yl]amino}-3-methoxybenzene- |
| | 1-sulfonamide |
| 2021. | 4-{[3-(4-{[(3R,4S)-1-[(2R)-2,3-dihydroxypropyl]-3-fluoropiperidin-4-yl]amino}- |
| | 1-(2,2,2-trifluoroethyl)-1H-indol-2-yl)prop-2-yn-1-yl]amino}-3-methoxybenzene- |
| | 1-sulfonamide |
| 2022. | (2R)-1-[(3RS,4SR)-3-fluoro-4-[(2-{3-[(4-methanesulfonyl-2- |
| | methoxyphenyl)amino]prop-1-yn-1-yl}-1-propyl-1H-indol-4-yl)amino]piperidin- |
| | 1-yl]-3-methoxypropan-2-ol |
| 2023. | 4-((3-(4-(((3S,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2024. | 4-((3-(4-(((3S,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2025. | 4-((3-(4-(((3R,4R)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoic acid |
| 2026. | (2R)-1-(acetyloxy)-3-[(3R,4S)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 2027. | N-[(2R)-2,3-dihydroxypropyl]-4-{[3-(4-{[(3RS,4SR)-3-fluoro-1-[(2R)-2-hydroxy- |
| | 3-methoxypropyl]piperidin-4-yl]amino}-1-(2,2,2-trifluoroethyl)-1H-indol-2- |
| | yl)prop-2-yn-1-yl]amino}-3-methoxybenzene-1-sulfonamide |
| 2028. | (2R)-1-(acetyloxy)-3-[(3S,4R)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 2029. | rac-N-(2-{bis[(pyridin-2-yl)methyl]amino}ethyl)-2-[(3R,4S)-3-fluoro-4-[(2-{3- |
| | [(4-methanesulfonyl-2-methoxyphenyl)amino]prop-1-yn-1-yl}-1-(2,2,2- |
| | trifluoroethyl)-1H-indol-4-yl)amino]piperidin-1-yl]acetamide |
| 2030. | 2-amino-1-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 2031. | 2-amino-1-[(3S,4R)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 2032. | 1-[(3R,4S)-3-fluoro-4-[(2-{3-[(4-methanesulfonyl-2-methoxyphenyl)amino]prop- |
| | 1-yn-1-yl}-1-(2,2,2-trifluoroethyl)-1H-indol-4-yl)amino]piperidin-1-yl]propan-2- |
| | ol |
| 2033. | 3-(4-(((3S,4R)-3-fluoro-1-methylpiperidin-4-yl)amino)-1-(2,2,2-trifluoroethyl)- |
| | 1H-indol-2-yl)prop-2-yn-1-ol |
| 2034. | (4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxybenzoyl)glycine |
| 2035. | methyl 4-{[3-(4-{[(3S,4R)-3-fluoro-1-(2-methoxyethyl)piperidin-4-yl]amino}-1- |
| | (2,2,2-trifluoroethyl)-1H-indol-2-yl)prop-2-yn-1-yl]amino}-3-methoxybenzoate |
| 2036. | 4-{[3-(4-{[(3S,4R)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoic acid |
| 2037. | methyl 4-{[3-(4-{[(3S,4R)-3-fluoro-1-(2-hydroxyethyl)piperidin-4-yl]amino}-1- |
| | (2,2,2-trifluoroethyl)-1H-indol-2-yl)prop-2-yn-1-yl]amino}-3-methoxybenzoate |
| 2038. | methyl 4-{[3-(4-{[(3S,4R)-3-fluoro-1-(2-hydroxypropyl)piperidin-4-yl]amino}-1- |
| | (2,2,2-trifluoroethyl)-1H-indol-2-yl)prop-2-yn-1-yl]amino}-3-methoxybenzoate |
| 2039. | methyl 4-{[3-(4-{[(3S,4R)-1-(carbamoylmethyl)-3-fluoropiperidin-4-yl]amino}-1- |
| | (2,2,2-trifluoroethyl)-1H-indol-2-yl)prop-2-yn-1-yl]amino}-3-methoxybenzoate |
| 2040. | methyl 4-{[3-(4-{[(3R,4S)-3-fluoro-1-(2-methoxyethyl)piperidin-4-yl]amino}-1- |
| | (2,2,2-trifluoroethyl)-1H-indol-2-yl)prop-2-yn-1-yl]amino}-3-methoxybenzoate |
| 2041. | methyl 4-{[3-(4-{[(3R,4S)-3-fluoro-1-(2-hydroxyethyl)piperidin-4-yl]amino}-1- |
| | (2,2,2-trifluoroethyl)-1H-indol-2-yl)prop-2-yn-1-yl]amino}-3-methoxybenzoate |
| 2042. | 4-{[3-(4-{[(3R,4S)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoic acid |
| 2043. | methyl 4-{[3-(4-{[(3R,4S)-3-fluoro-1-(2-hydroxypropyl)piperidin-4-yl]amino}-1- |
| | (2,2,2-trifluoroethyl)-1H-indol-2-yl)prop-2-yn-1-yl]amino}-3-methoxybenzoate |
| 2044. | N-[(3S,4R)-3-fluoro-1-methylpiperidin-4-yl]-2-{3-[(2-methoxy-4-{2-oxa-6- |
| | azaspiro[3.3]heptane-6-carbonyl}phenyl)amino]prop-1-yn-1-yl}-1-(2,2,2- |
| | trifluoroethyl)-1H-indol-4-amine |
| 2045. | N-[(3S,4R)-3-fluoro-1-methylpiperidin-4-yl]-2-{3-[(2-methoxy-4-{7-oxa-2- |
| | azaspiro[3.5]nonane-2-carbonyl}phenyl)amino]prop-1-yn-1-yl}-1-(2,2,2- |
| | trifluoroethyl)-1H-indol-4-amine |
| 2046. | 4-{[3-(4-{[(3S,4R)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoic acid |
| 2047. | N-[(3S,4R)-3-fluoro-1-methylpiperidin-4-yl]-2-(3-{[2-methoxy-4-(morpholine-4- |
| | carbonyl)phenyl]amino}prop-1-yn-1-yl)-1-(2,2,2-trifluoroethyl)-1H-indol-4-amine |
| 2048. | N-[(3S,4R)-3-fluoro-1-methylpiperidin-4-yl]-2-(3-{[2-methoxy-4-(4- |
| | methylpiperazine-1-carbonyl)phenyl]amino}prop-1-yn-1-yl)-1-(2,2,2- |
| | trifluoroethyl)-1H-indol-4-amine |
| 2049. | 4-{[3-(4-{[(3R,4S)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoic acid |
| 2050. | 4-{[3-(4-{[(3R,4S)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoic acid |
| 2051. | methyl 4-{[3-(4-{[(3S,4R)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoate |
| 2052. | methyl 4-{[3-(4-{[(3R,4S)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoate |
| 2053. | methyl 4-{[3-(4-{[(3R,4S)-1-(carbamoylmethyl)-3-fluoropiperidin-4-yl]amino}-1- |
| | (2,2,2-trifluoroethyl)-1H-indol-2-yl)prop-2-yn-1-yl]amino}-3-methoxybenzoate |
| 2054. | 2-((3R,4S)-3-fluoro-4-((2-(3-((2-methoxy-4-(methylsulfonyl)phenyl)amino)prop- |
| | 1-yn-1-yl)-1-(2,2,2-trifluoroethyl)-1H-indol-4-yl)amino)piperidin-1-yl)acetamide |
| 2055. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-(1,3-thiazol-2- |
| | yl)benzamide |
| 2056. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-(1- |
| | methylpiperidin-4-yl)benzamide |
| 2057. | 1-(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxybenzoyl)piperidin-4-ol |
| 2058. | 4-{[3-(4-{[(3S,4R)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoic acid |
| 2059. | 4-{[3-(4-{[(3S,4R)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoic acid |
| 2060. | tert-butyl (3S,4R)-4-{[2-(3-{[4-(ethanesulfonyl)-2-methoxyphenyl]amino}prop-1- |
| | yn-1-yl)-1-(2,2,2-trifluoroethyl)-1H-indol-4-yl]amino}-3-fluoropiperidine-1- |
| | carboxylate |
| 2061. | 2-(3-((4-(ethylsulfonyl)-2-methoxyphenyl)amino)prop-1-yn-1-yl)-N-((3S,4R)-3- |
| | fluoropiperidin-4-yl)-1-(2,2,2-trifluoroethyl)-1H-indol-4-amine |
| 2062. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-[2-(morpholin- |
| | 4-yl)ethyl]benzamide |
| 2063. | 4-{[3-(4-{[(3R,4S)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2064. | 4-{[3-(4-{[(3S,4R)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2065. | 4-{[3-(4-{[(3R,4S)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoic acid |
| 2066. | tert-butyl (3S,4R)-4-[(2-{3-[(2-ethoxy-4-methanesulfonylphenyl)amino]prop-1- |
| | yn-1-yl}-1-(2,2,2-trifluoroethyl)-1H-indol-4-yl)amino]-3-fluoropiperidine-1- |
| | carboxylate |
| 2067. | (2S)-2-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]pentanedioic acid |
| 2068. | (2S)-4-carbamoyl-2-[(4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxyphenyl)formamido]butanoic acid |
| 2069. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzene-1- |
| | sulfonic acid |
| 2070. | 1,5-dimethyl (2S)-2-[(4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxyphenyl)formamido]pentanedioate |
| 2071. | 2-[3-({4-[4-(dimethylamino)piperidine-1-carbonyl]-2- |
| | methoxyphenyl}amino)prop-1-yn-1-yl]-N-[(3S,4R)-3-fluoro-1-methylpiperidin-4- |
| | yl]-1-(2,2,2-trifluoroethyl)-1H-indol-4-amine |
| 2072. | 4-{[3-(4-{[(3S,4R)-1-(carboxymethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2073. | 2-(3-{[4-(ethanesulfonyl)-2-methoxyphenyl]amino}prop-1-yn-1-yl)-N-[(3S,4R)-3- |
| | fluoropiperidin-4-yl]-1-(2,2,2-trifluoroethyl)-1H-indol-4-amine |
| 2074. | (1R,2R,4S)-2-fluoro-N¹-(2-{3-[(4-methanesulfonyl-2- |
| | methoxyphenyl)amino]prop-1-yn-1-yl}-1-(2,2,2-trifluoroethyl)-1H-indol-4- |
| | yl)cyclohexane-1,4-diamine |
| 2075. | (1R,2R,4S)-2-fluoro-N¹-(2-{3-[(4-methanesulfonyl-2- |
| | methoxyphenyl)amino]prop-1-yn-1-yl}-1-(2,2,2-trifluoroethyl)-1H-indol-4-yl)- |
| | N⁴-methylcyclohexane-1,4-diamine |
| 2076. | (1S,3R,4R)-3-fluoro-N⁴-(2-{3-[(4-methanesulfonyl-2- |
| | methoxyphenyl)amino]prop-1-yn-1-yl}-1-(2,2,2-trifluoroethyl)-1H-indol-4-yl)- |
| | N¹,N¹-dimethylcyclohexane-1,4-diamine |
| 2077. | 2-(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxybenzenesulfonyl)ethan-1-ol |
| 2078. | N-ethyl-4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 2079. | 2-{3-[(2-ethoxy-4-methanesulfonylphenyl)amino]prop-1-yn-1-yl}-N-[(3S,4R)-3- |
| | fluoropiperidin-4-yl]-1-(2,2,2-trifluoroethyl)-1H-indol-4-amine |
| 2080. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N,N- |
| | dimethylbenzamide |
| 2081. | N-ethyl-4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxy-N- |
| | methylbenzamide |
| 2082. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-methyl-N- |
| | (propan-2-yl)benzamide |
| 2083. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-(2- |
| | methoxyethyl)benzamide |
| 2084. | N-[2-(diethylamino)ethyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2085. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-N-(2-hydroxyethyl)-3- |
| | methoxybenzamide |
| 2086. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N- |
| | [(2R,3R,4R,5S,6R)-2,4,5-trihydroxy-6-(hydroxymethyl)oxan-3-yl]benzamide |
| 2087. | 4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-N-(1-hydroxypropan-2-yl)-3- |
| | methoxybenzamide |
| 2088. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-N-[(2R)-1-hydroxypropan-2- |
| | yl]-3-methoxybenzamide |
| 2089. | N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]cyclopropanecarboxamide |
| 2090. | (1R,2R)-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-2-phenylcyclopropane-1- |
| | carboxamide |
| 2091. | N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1-methyl-1H-pyrrole-3-carboxamide |
| 2092. | 1-ethyl-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1H-pyrrole-3-carboxamide |
| 2093. | 1-tert-butyl-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1H-pyrrole-3-carboxamide |
| 2094. | methyl (2S)-4-carbamoyl-2-[(4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxyphenyl)formamido]butanoate |
| 2095. | 4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-N-((R)-2-hydroxypropyl)-3- |
| | methoxybenzamide |
| 2096. | rac-4-{[3-(4-{[(3R,4S)-1-ethyl-3-fluoropiperidin-4-yl]amino}-1-(2,2,2- |
| | trifluoroethyl)-1H-indol-2-yl)prop-2-yn-1-yl]amino}-3-methoxybenzoic acid |
| 2097. | 4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-N-((S)-2-hydroxypropyl)-3- |
| | methoxybenzamide |
| 2098. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-N-(2-hydroxy-3- |
| | methoxypropyl)-3-methoxybenzamide |
| 2099. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-N-[(2S)-2-hydroxypropyl]-3- |
| | methoxybenzamide |
| 2100. | N-(2,3-dihydroxypropyl)-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2101. | N-[(2R)-2,3-dihydroxypropyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2102. | N-[(2S)-2,3-dihydroxypropyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2103. | N-(1,5-dihydroxypentan-3-yl)-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2104. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-N-(3-hydroxy-2- |
| | methoxypropyl)-3-methoxybenzamide |
| 2105. | 1-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]-3-[(2-methylpropanoyl)oxy]propan-2-yl 2- |
| | methylpropanoate |
| 2106. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxy-N-[(2-oxo-1,3- |
| | dioxolan-4-yl)methyl]benzamide |
| 2107. | 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-N-(2-methanesulfonylethyl)- |
| | 3-methoxybenzamide |
| 2108. | 1-(acetyloxy)-3-[(4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3- |
| | methoxyphenyl)formamido]propan-2-yl acetate |
| 2109. | 1-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]-3-(propanoyloxy)propan-2-yl propanoate |
| 2110. | 2-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]propyl 2-methylpropanoate |
| 2111. | (S)-5-ethoxy-2-(4-((3-(4-(((3S,4R)-3-fluoro-1-methylpiperidin-4-yl)amino)-1- |
| | (2,2,2-trifluoroethyl)-1H-indol-2-yl)prop-2-yn-1-yl)amino)-3- |
| | methoxybenzamido)-5-oxopentanoic acid |
| 2112. | (4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxybenzoyl)-L- |
| | glutamine |
| 2113. | (S)-2-(4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxybenzamido)-5- |
| | methoxy-5-oxopentanoic acid |
| 2114. | (S)-1-((3S,4S)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2115. | (S)-1-((3R,4R)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2116. | (S)-1-((3R,4S)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2117. | (S)-1-((3S,4R)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2118. | 1-((3S,4S)-3-fluoro-4-((2-(3-((2-methoxy-4-(methylsulfonyl)phenyl)amino)prop- |
| | 1-yn-1-yl)-1-(2,2,2-trifluoroethyl)-1H-indol-4-yl)amino)piperidin-1-yl)-3- |
| | methoxypropan-2-ol |
| 2119. | 1-((3R,4S)-3-fluoro-4-((2-(3-((2-methoxy-4-(methylsulfonyl)phenyl)amino)prop- |
| | 1-yn-1-yl)-1-(2,2,2-trifluoroethyl)-1H-indol-4-yl)amino)piperidin-1-yl)-3- |
| | methoxypropan-2-ol |
| 2120. | (R)-1-((3S,4S)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2121. | (R)-1-((3R,4R)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2122. | (R)-1-((3R,4S)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2123. | (R)-1-((3S,4R)-3-fluoro-4-((2-(3-((2-methoxy-4- |
| | (methylsulfonyl)phenyl)amino)prop-1-yn-1-yl)-1-(2,2,2-trifluoroethyl)-1H-indol- |
| | 4-yl)amino)piperidin-1-yl)-3-methoxypropan-2-ol |
| 2124. | 4-((3-(4-(((3R,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzenesulfonamide |
| 2125. | 1-(3,3-difluoro-4-((2-(3-((2-methoxy-4-(methylsulfonyl)phenyl)amino)prop-1-yn- |
| | 1-yl)-1-(2,2,2-trifluoroethyl)-1H-indol-4-yl)amino)piperidin-1-yl)-3- |
| | methoxypropan-2-ol |
| 2126. | 2-{3-[(4-methanesulfonyl-2-methoxyphenyl)amino]prop-1-yn-1-yl}-N-(1- |
| | methylazepan-4-yl)-1-(2,2,2-trifluoroethyl)-1H-indol-4-amine |
| 2127. | 1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]azepan-1-yl}-3-methoxypropan-2-ol |
| 2128. | 2-{3-[(4-methanesulfonyl-2-methoxyphenyl)amino]prop-1-yn-1-yl}-N-{8-methyl- |
| | 8-azabicyclo[3.2.1]octan-3-yl}-1-(2,2,2-trifluoroethyl)-1H-indol-4-amine |
| 2129. | 2-(3-{[4-methanesulfonyl-2-(trifluoromethoxy)phenyl]amino}prop-1-yn-1-yl)-N- |
| | (oxan-4-yl)-1-(2,2,2-trifluoroethyl)-1H-indol-4-amine |
| 2130. | 3-methoxy-4-[(3-{4-[(oxan-4-yl)amino]-1-(2,2,2-trifluoroethyl)-1H-indol-2- |
| | yl}prop-2-yn-1-yl)amino]-N-(1,2-oxazol-3-yl)benzene-1-sulfonamide |
| 2131. | 3-methoxy-N-(oxan-4-yl)-4-[(3-{4-[(oxan-4-yl)amino]-1-(2,2,2-trifluoroethyl)- |
| | 1H-indol-2-yl}prop-2-yn-1-yl)amino]benzene-1-sulfonamide |
| 2132. | 3-methoxy-N-(5-methyl-1,2-oxazol-3-yl)-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2133. | 2-{4-methoxy-5-[(3-{4-[(oxan-4-yl)amino]-1-(2,2,2-trifluoroethyl)-1H-indol-2- |
| | yl}prop-2-yn-1-yl)amino]pyridin-2-yl}-2-methylpropanenitrile |
| 2134. | N-(2-hydroxyethyl)-3-methoxy-N-methyl-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2135. | 6-fluoro-2-{3-[(4-methanesulfonyl-2-methoxyphenyl)amino]prop-1-yn-1-yl}-N- |
| | (oxan-4-yl)-1-(2,2,2-trifluoroethyl)-1H-indol-4-amine |
| 2136. | 4-[(3-{6-fluoro-4-[(oxan-4-yl)amino]-1-(2,2,2-trifluoroethyl)-1H-indol-2-yl}prop- |
| | 2-yn-1-yl)amino]-3-methoxybenzene-1-sulfonamide |
| 2137. | 3-methoxy-N,N-dimethyl-4-[(3-{4-[(oxan-4-yl)amino]-1-(2,2,2-trifluoroethyl)- |
| | 1H-indol-2-yl}prop-2-yn-1-yl)amino]benzene-1-sulfonamide |
| 2138. | 3-methoxy-N-(2-methoxyethyl)-N-methyl-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2139. | 1-(4-{3-methoxy-4-[(3-{4-[(oxan-4-yl)amino]-1-(2,2,2-trifluoroethyl)-1H-indol-2- |
| | yl}prop-2-yn-1-yl)amino]benzenesulfonyl}piperazin-1-yl)ethan-1-one |
| 2140. | 2-(3-{[2-methoxy-4-(morpholine-4-sulfonyl)phenyl]amino}prop-1-yn-1-yl)-N- |
| | (oxan-4-yl)-1-(2,2,2-trifluoroethyl)-1H-indol-4-amine |
| 2141. | 3-methoxy-N-methyl-4-[(3-{4-[(oxan-4-yl)amino]-1-(2,2,2-trifluoroethyl)-1H- |
| | indol-2-yl}prop-2-yn-1-yl)amino]benzamide |
| 2142. | 3-methoxy-4-((3-(4-((tetrahydro-2H-pyran-4-yl)amino)-1-(2,2,2-trifluoroethyl)- |
| | 1H-indol-2-yl)prop-2-yn-1-yl)amino)benzoic acid |
| 2143. | methyl 3-methoxy-4-((3-(4-((tetrahydro-2H-pyran-4-yl)amino)-1-(2,2,2- |
| | trifluoroethyl)-1H-indol-2-yl)prop-2-yn-1-yl)amino)benzoate |
| 2144. | 3-methoxy-4-((3-(4-((tetrahydro-2H-pyran-4-yl)amino)-1-(2,2,2-trifluoroethyl)- |
| | 1H-indol-2-yl)prop-2-yn-1-yl)amino)benzamide |
| 2145. | 3-methoxy-N-(2-methoxyethyl)-N-methyl-4-((3-(4-((tetrahydro-2H-pyran-4- |
| | yl)amino)-1-(2,2,2-trifluoroethyl)-1H-indol-2-yl)prop-2-yn-1- |
| | yl)amino)benzenesulfonamide |
| 2146. | N,N-bis(2-hydroxyethyl)-3-methoxy-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2147. | 3-methoxy-N-methyl-4-[(3-{4-[(oxan-4-yl)amino]-1-(2,2,2-trifluoroethyl)-1H- |
| | indol-2-yl}prop-2-yn-1-yl)amino]benzene-1-sulfonamide |
| 2148. | 5-methanesulfonyl-2-[(3-{4-[(oxan-4-yl)amino]-1-(2,2,2-trifluoroethyl)-1H-indol- |
| | 2-yl}prop-2-yn-1-yl)amino]phenol |
| 2149. | 3-methoxy-4-[(3-{5-methoxy-4-[(oxan-4-yl)amino]-1-(2,2,2-trifluoroethyl)-1H- |
| | indol-2-yl}prop-2-yn-1-yl)amino]benzene-1-sulfonamide |
| 2150. | 2-[3-({2-methoxy-4-[(4-methylpiperazin-1-yl)sulfonyl]phenyl}amino)prop-1-yn- |
| | 1-yl]-N-(oxan-4-yl)-1-(2,2,2-trifluoroethyl)-1H-indol-4-amine |
| 2151. | 2-{3-[(4-methanesulfonyl-2-methoxyphenyl)amino]prop-1-yn-1-yl}-5-methoxy- |
| | N-(oxan-4-yl)-1-(2,2,2-trifluoroethyl)-1H-indol-4-amine |
| 2152. | 3-methoxy-4-[(3-{6-methoxy-4-[(oxan-4-yl)amino]-1-(2,2,2-trifluoroethyl)-1H- |
| | indol-2-yl}prop-2-yn-1-yl)amino]benzene-1-sulfonamide |
| 2153. | 5-methanesulfonyl-2-[(3-{4-[(oxan-4-yl)amino]-1-(2,2,2-trifluoroethyl)-1H-indol- |
| | 2-yl}prop-2-yn-1-yl)amino]phenyl propanoate |
| 2154. | N-(2-hydroxyethyl)-3-methoxy-4-((3-(4-((tetrahydro-2H-pyran-4-yl)amino)-1- |
| | (2,2,2-trifluoroethyl)-1H-indol-2-yl)prop-2-yn-1-yl)amino)benzenesulfonamide |
| 2155. | 2-{3-[(4-methanesulfonyl-2-methoxyphenyl)amino]prop-1-yn-1-yl}-N-[(2S,4R)- |
| | 2-methyloxan-4-yl]-1-(2,2,2-trifluoroethyl)-1H-indol-4-amine |
| 2156. | 2-{3-[(4-methanesulfonyl-2-methoxyphenyl)amino]prop-1-yn-1-yl}-N-[(2S,4S)-2- |
| | methyloxan-4-yl]-1-(2,2,2-trifluoroethyl)-1H-indol-4-amine |
| 2157. | 3-methoxy-4-{[3-(4-{[(2S,4R)-2-methyloxan-4-yl]amino}-1-(2,2,2-trifluoroethyl)- |
| | 1H-indol-2-yl)prop-2-yn-1-yl]amino}benzene-1-sulfonamide |
| 2158. | 3-methoxy-4-{[3-(4-{[(2S,4S)-2-methyloxan-4-yl]amino}-1-(2,2,2-trifluoroethyl)- |
| | 1H-indol-2-yl)prop-2-yn-1-yl]amino}benzene-1-sulfonamide |
| 2159. | 3-methoxy-4-{[3-(4-{[(2S,4R)-2-methyloxan-4-yl]amino}-1-(2,2,2-trifluoroethyl)- |
| | 1H-indol-2-yl)prop-2-yn-1-yl]amino}benzamide |
| 2160. | 3-methoxy-4-{[3-(4-{[(2S,4S)-2-methyloxan-4-yl]amino}-1-(2,2,2-trifluoroethyl)- |
| | 1H-indol-2-yl)prop-2-yn-1-yl]amino}benzamide |
| 2161. | rac-N-[(3R,4R)-3-fluorooxan-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 2162. | 2-(3-((2-methoxy-4-(methylsulfonyl)phenyl)amino)prop-1-yn-1-yl)-N-((3R,4S)-3- |
| | methoxytetrahydro-2H-pyran-4-yl)-1-(2,2,2-trifluoroethyl)-1H-indol-4-amine |
| 2163. | 2-(3-((2-methoxy-4-(methylsulfonyl)phenyl)amino)prop-1-yn-1-yl)-N-((3S,4S)-3- |
| | methoxytetrahydro-2H-pyran-4-yl)-1-(2,2,2-trifluoroethyl)-1H-indol-4-amine |
| 2164. | N-[(3S,4S)-3-fluorooxan-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 2165. | N-[(3R,4R)-3-fluorooxan-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 2166. | 2-{3-[(4-methanesulfonyl-2-methoxyphenyl)amino]prop-1-yn-1-yl}-N- |
| | [(2R,4R,6S)-2,6-dimethyloxan-4-yl]-1-(2,2,2-trifluoroethyl)-1H-indol-4-amine |
| 2167. | 2-{3-[(4-methanesulfonyl-2-methoxyphenyl)amino]prop-1-yn-1-yl}-N- |
| | [(2R,4S,6S)-2,6-dimethyloxan-4-yl]-1-(2,2,2-trifluoroethyl)-1H-indol-4-amine |
| 2168. | 3-methoxy-4-{[3-(4-{[(2R,4R,6S)-2,6-dimethyloxan-4-yl]amino}-1-(2,2,2- |
| | trifluoroethyl)-1H-indol-2-yl)prop-2-yn-1-yl]amino}benzamide |
| 2169. | 3-methoxy-4-{[3-(4-{[(2R,4S,6S)-2,6-dimethyloxan-4-yl]amino}-1-(2,2,2- |
| | trifluoroethyl)-1H-indol-2-yl)prop-2-yn-1-yl]amino}benzamide |
| 2170. | 3-methoxy-4-{[3-(4-{[(2R,4S,6S)-2,6-dimethyloxan-4-yl]amino}-1-(2,2,2- |
| | trifluoroethyl)-1H-indol-2-yl)prop-2-yn-1-yl]amino}benzene-1-sulfonamide |
| 2171. | 3-methoxy-4-{[3-(4-{[(2R,4R,6S)-2,6-dimethyloxan-4-yl]amino}-1-(2,2,2- |
| | trifluoroethyl)-1H-indol-2-yl)prop-2-yn-1-yl]amino}benzene-1-sulfonamide |
| 2172. | 4-((2-(3-((4-methoxypyridin-3-yl)amino)prop-1-yn-1-yl)-1-(2,2,2-trifluoroethyl)- |
| | 1H-indol-4-yl)amino)tetrahydro-2H-thiopyran 1,1-dioxide |
| 2173. | 2-{5-[(3-{4-[(1, 1-dioxo-1λ⁶-thian-4-yl)amino]-1-(2,2,2-trifluoroethyl)-1H-indol-2- |
| | yl}prop-2-yn-1-yl)amino]-4-methoxypyridin-2-yl}-2-methylpropanenitrile |
| 2174. | 4-{[2-(3-{[4-methanesulfonyl-2-(trifluoromethoxy)phenyl]amino}prop-1-yn-1- |
| | yl)-1-(2,2,2-trifluoroethyl)-1H-indol-4-yl]amino}-1λ⁶-thiane-1,1-dione |
| 2175. | 4-{[2-(3-{[2-methoxy-4-(morpholine-4-sulfonyl)phenyl]amino}prop-1-yn-1-yl)-1- |
| | (2,2,2-trifluoroethyl)-1H-indol-4-yl]amino}-1λ⁶-thiane-1,1-dione |
| 2176. | 4-({2-[3-({2-[2-(dimethylamino)ethoxy]-4-methanesulfonylphenyl}amino)prop-1- |
| | yn-1-yl]-1-(2,2,2-trifluoroethyl)-1H-indol-4-yl}amino)-1λ⁶-thiane-1,1-dione |
| 2177. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-N-(2-hydroxyethyl)-3-methoxy-N-methylbenzene-1- |
| | sulfonamide |
| 2178. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxy-N-(1,2-oxazol-3-yl)benzene-1-sulfonamide |
| 2179. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxy-N-(5-methyl-1,2-oxazol-3-yl)benzene-1- |
| | sulfonamide |
| 2180. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxy-N-(oxan-4-yl)benzene-1-sulfonamide |
| 2181. | N-(2,3-dihydroxypropyl)-4-[(3-{4-[(1,1-dioxo-1λ⁶-thian-4-yl)amino]-1-(2,2,2- |
| | trifluoroethyl)-1H-indol-2-yl}prop-2-yn-1-yl)amino]-3-methoxybenzene-1- |
| | sulfonamide |
| 2182. | N-[2-(dimethylamino)ethyl]-4-[(3-{4-[(1,1-dioxo-1λ⁶-thian-4-yl)amino]-1-(2,2,2- |
| | trifluoroethyl)-1H-indol-2-yl}prop-2-yn-1-yl)amino]-3-methoxy-N- |
| | methylbenzene-1-sulfonamide |
| 2183. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxy-N,N-dimethylbenzene-1-sulfonamide |
| 2184. | 4-({2-[3-({4-[(4-acetylpiperazin-1-yl)sulfonyl]-2-methoxyphenyl}amino)prop-1- |
| | yn-1-yl]-1-(2,2,2-trifluoroethyl)-1H-indol-4-yl}amino)-1λ⁶-thiane-1,1-dione |
| 2185. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxy-N-(2-methoxyethyl)-N-methylbenzene-1- |
| | sulfonamide |
| 2186. | 4-[(2-{3-[(2-methoxy-4-{2-oxa-6-azaspiro[3.3]heptane-6- |
| | sulfonyl}phenyl)amino]prop-1-yn-1-yl}-1-(2,2,2-trifluoroethyl)-1H-indol-4- |
| | yl)amino]-1λ⁶-thiane-1,1-dione |
| 2187. | 4-({2-[3-({2-methoxy-4-[(4-methylpiperazin-1-yl)sulfonyl]phenyl}amino)prop-1- |
| | yn-1-yl]-1-(2,2,2-trifluoroethyl)-1H-indol-4-yl}amino)-1λ⁶-thiane-1,1-dione |
| 2188. | 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxy-N-methylbenzamide |
| 2189. | 4-((2-(3-((4-methoxy-6-(methylsulfonyl)pyridin-3-yl)amino)prop-1-yn-1-yl)-1- |
| | (2,2,2-trifluoroethyl)-1H-indol-4-yl)amino)tetrahydro-2H-thiopyran 1,1-dioxide |
| 2190. | 4-((2-(3-((4-(methylsulfonyl)-2-(2,2,2-trifluoroethoxy)phenyl)amino)prop-1-yn-1- |
| | yl)-1-(2,2,2-trifluoroethyl)-1H-indol-4-yl)amino)tetrahydro-2H-thiopyran 1,1- |
| | dioxide |
| 2191. | 2-(2-((3-(4-((1,1-dioxidotetrahydro-2H-thiopyran-4-yl)amino)-1-(2,2,2- |
| | trifluoroethyl)-1H-indol-2-yl)prop-2-yn-1-yl)amino)-5- |
| | (methylsulfonyl)phenoxy)acetamide |
| 2192. | 2-(2-((3-(4-((1,1-dioxidotetrahydro-2H-thiopyran-4-yl)amino)-1-(2,2,2- |
| | trifluoroethyl)-1H-indol-2-yl)prop-2-yn-1-yl)amino)-5- |
| | (methylsulfonyl)phenoxy)acetamide |
| 2193. | N-(2,3-dihydroxypropyl)-4-((3-(4-((1,1-dioxidotetrahydro-2H-thiopyran-4- |
| | yl)amino)-1-(2,2,2-trifluoroethyl)-1H-indol-2-yl)prop-2-yn-1-yl)amino)-3- |
| | methoxybenzenesulfonamide |
| 2194. | 3-methoxy-4-((3-(4-(3-(1-methylpiperidin-4-yl)ureido)-1-(2,2,2-trifluoroethyl)- |
| | 1H-indol-2-yl)prop-2-yn-1-yl)amino)benzamide |

TABLE 2
Aryl-linked indole compounds of the disclosure.

| Mol # | Structure IUPAC name |
|---|---|
| 2195. | 4-{4-[(1,1-dioxo-1λ⁶-thian-4-yl)amino]-1-(2,2,2-trifluoroethyl)-1H-indol-2- |
| | yl}benzamide |
| 2196. | 4-({2-[4-(aminomethyl)phenyl]-1-(2,2,2-trifluoroethyl)-1H-indol-4- |
| | yl}amino)-1λ⁶-thiane-1,1-dione |
| 2197. | 4-[(2-{4-[(methylamino)methyl]phenyl}-1-(2,2,2-trifluoroethyl)-1H-indol-4- |
| | yl)amino]-1λ⁶-thiane-1,1-dione |
| 2198. | tert-butyl N-[(4-{4-[(1,1-dioxo-1λ⁶-thian-4-yl)amino]-1-(2,2,2-trifluoroethyl)- |
| | 1H-indol-2-yl}phenyl)methyl]-N-methylcarbamate |
| 2199. | 4-{4-[(1,1-dioxo-1λ⁶-thian-4-yl)amino]-1-(2,2,2-trifluoroethyl)-1H-indol-2-yl}- |
| | N-methylbenzamide |
| 2200. | tert-butyl N-[(4-{4-[(1,1-dioxo-1λ⁶-thian-4-yl)amino]-1-(2,2,2-trifluoroethyl)- |
| | 1H-indol-2-yl}phenyl)methyl]carbamate |
| 2201. | 2-(5-{[(4-{4-[(1,1-dioxo-1λ⁶-thian-4-yl)amino]-1-(2,2,2-trifluoroethyl)-1H- |
| | indol-2-yl}phenyl)methyl]amino}pyridin-2-yl)-2-methylpropanenitrile |
| 2202. | 4-{[2-(4-{[(4-methanesulfonylphenyl)amino]methyl}phenyl)-1-(2,2,2- |
| | trifluoroethyl)-1H-indol-4-yl]amino}-1λ⁶-thiane-1,1-dione |
| 2203. | 4-[(2-{4-[(phenylamino)methyl]phenyl}-1-(2,2,2-trifluoroethyl)-1H-indol-4- |
| | yl)amino]-1λ⁶-thiane-1,1-dione |
| 2204. | 3-{4-[(1,1-dioxo-1λ⁶-thian-4-yl)amino]-1-(2,2,2-trifluoroethyl)-1H-indol-2- |
| | yl}benzonitrile |
| 2205. | 4-{[2-(2-fluoro-4-methylphenyl)-1-(2,2,2-trifluoroethyl)-1H-indol-4- |
| | yl]amino}-1λ⁶-thiane-1,1-dione |
| 2206. | 4-{[2-(3-chlorophenyl)-1-(2,2,2-trifluoroethyl)-1H-indol-4-yl]amino}-1λ⁶- |
| | thiane-1,1-dione |
| 2207. | -{[2-(3-methoxyphenyl)-1-(2,2,2-trifluoroethyl)-1H-indol-4-yl]amino}-1λ⁶- |
| | thiane-1,1-dione |
| 2208. | 4-{[2-(4-chlorophenyl)-1-(2,2,2-trifluoroethyl)-1H-indol-4-yl]amino}-1λ⁶- |
| | thiane-1,1-dione |
| 2209. | 4-tert-butyl-N-[(4-{4-[(1,1-dioxo-1λ⁶-thian-4-yl)amino]-1-(2,2,2- |
| | trifluoroethyl)-1H-indol-2-yl}phenyl)methyl]benzamide |
| 2210. | 4-cyano-N-[(4-{4-[(1,1-dioxo-1λ⁶-thian-4-yl)amino]-1-(2,2,2-trifluoroethyl)- |
| | 1H-indol-2-yl}phenyl)methyl]benzamide |
| 2211. | 4-chloro-N-[(4-{4-[(1,1-dioxo-1λ⁶-thian-4-yl)amino]-1-(2,2,2-trifluoroethyl)- |
| | 1H-indol-2-yl}phenyl)methyl]benzamide |
| 2212. | 3-[6-(1-cyano-1-methylethyl)pyridin-3-yl]-1-[(4-{4-[(1,1-dioxo-1λ⁶-thian-4- |
| | yl)amino]-1-(2,2,2-trifluoroethyl)-1H-indol-2-yl}phenyl)methyl]urea |
| 2213. | 3-[(4-{4-[(1,1-dioxo-1λ⁶-thian-4-yl)amino]-1-(2,2,2-trifluoroethyl)-1H-indol-2- |
| | yl}phenyl)methyl]-1-phenylurea |
| 2214. | 3-{4-[(1,1-dioxo-1λ⁶-thian-4-yl)amino]-1-(2,2,2-trifluoroethyl)-1H-indol-2- |
| | yl}benzoic acid |
| 2215. | 4-({2-[3-(dimethylamino)phenyl]-1-(2,2,2-trifluoroethyl)-1H-indol-4- |
| | yl}amino)-1λ⁶-thiane-1,1-dione |
| 2216. | 3-{4-[(1,1-dioxo-1λ⁶-thian-4-yl)amino]-1-(2,2,2-trifluoroethyl)-1H-indol-2-yl}- |
| | N-methylbenzamide |
| 2217. | 4-{4-[(11-dioxo-1λ⁶-thian-4-yl)amino]-1-(2,2,2-trifluoroethyl)-1H-indol-2- |
| | yl}benzoic acid |
| 2218. | 4-[(2-{4-[(morpholin-4-yl)methyl]phenyl}-1-(2,2,2-trifluoroethyl)-1H-indol-4- |
| | yl)amino]-1λ⁶-thiane-1,1-dione |
| 2219. | methyl N-(3-{4-[(1,1-dioxo-1λ⁶-thian-4-yl)amino]-1-(2,2,2-trifluoroethyl)-1H- |
| | indol-2-yl}phenyl)carbamate |
| 2220. | 1-(4-{4-[(1,1-dioxo-1λ⁶-thian-4-yl)amino]-1-(2,2,2-trifluoroethyl)-1H-indol-2- |
| | yl}phenyl)cyclopropane-1-carbonitrile |
| 2221. | 4-({2-[4-(hydroxymethyl)phenyl]-1-(2,2,2-trifluoroethyl)-1H-indol-4- |
| | yl}amino)-1λ⁶-thiane-1,1-dione |
| 2222. | 1-[(4-{4-[(1,1-dioxo-1λ⁶-thian-4-yl)amino]-1-(2,2,2-trifluoroethyl)-1H-indol-2- |
| | yl}phenyl)methyl]-3-(4-methanesulfonylphenyl)urea |
| 2223. | 4-{[2-(4-{[(6-methanesulfonylpyridin-3-yl)amino]methyl}phenyl)-1-(2,2,2- |
| | trifluoroethyl)-1H-indol-4-yl]amino}-1λ⁶-thiane-1,1-dione |
| 2224. | 2-(4-{[(4-methanesulfonylphenyl)amino]methyl}phenyl)-N-[1-(oxan-4- |
| | yl)piperidin-4-yl]-1-(2,2,2-trifluoroethyl)-1H-indol-4-amine |
| 2225. | 2-(4-{[(6-methylpyridin-3-yl)amino]methyl}phenyl)-N-[1-(oxan-4- |
| | yl)piperidin-4-yl]-1-(2,2,2-trifluoroethyl)-1H-indol-4-amine |
| 2226. | 2-(4-{[(4-chlorophenyl)amino]methyl}phenyl)-N-[1-(oxan-4-yl)piperidin-4- |
| | yl]-1-(2,2,2-trifluoroethyl)-1H-indol-4-amine |
| 2227. | 2-(4-{[(4-methoxyphenyl)amino]methyl}phenyl)-N-[1-(oxan-4-yl)piperidin-4- |
| | yl]-1-(2,2,2-trifluoroethyl)-1H-indol-4-amine |
| 2228. | 2-(4-{[(3-chlorophenyl)amino]methyl}phenyl)-N-[1-(oxan-4-yl)piperidin-4- |
| | yl]-1-(2,2,2-trifluoroethyl)-1H-indol-4-amine |
| 2229. | 6-methyl-N-{[4-(5-{[(1-methylpiperidin-4-yl)amino]methyl}-1-(2,2,2- |
| | trifluoroethyl)-1H-indol-2-yl)phenyl]methyl}pyridin-3-amine |
| 2230. | N-{[2-(4-{[(4-methanesulfonylphenyl)amino]methyl}phenyl)-1-(2,2,2- |
| | trifluoroethyl)-1H-indol-5-yl]methyl}-1-methylpiperidin-4-amine |
| 2231. | 2-(5-amino-[1,1′-biphenyl]-3-yl)-N-(1-methylpiperidin-4-yl)-1-(2,2,2- |
| | trifluoroethyl)-1H-indol-4-amine |
| 2232. | 2-{4-[amino(phenyl)methyl]phenyl}-N-(1-methylpiperidin-4-yl)-1-(2,2,2- |
| | trifluoroethyl)-1H-indol-4-amine |
| 2233. | 2-(4-(amino(cyclohexyl)methyl)phenyl)-N-(1-methylpiperidin-4-yl)-1-(2,2,2- |
| | trifluoroethyl)-1H-indol-4-amine |
| 2234. | 2-{4-[(cyclopentylamino)methyl]phenyl}-N-(1-methylpiperidin-4-yl)-1-(2,2,2- |
| | trifluoroethyl)-1H-indol-4-amine |
| 2235. | 2-(4-{1-[(4-methanesulfonylphenyl)amino]ethyl}phenyl)-N-(1- |
| | methylpiperidin-4-yl)-1-(2,2,2-trifluoroethyl)-1H-indol-4-amine |
| 2236. | (+/−)-2-{4-[(cyclopropylamino)methyl]phenyl}-N-[(3R,4S)-3-fluoro-1- |
| | methylpiperidin-4-yl]-1-(2,2,2-trifluoroethyl)-1H-indol-4-amine |
| 2237. | (+/−)-N-[(3R,4S)-3-fluoro-1-methylpiperidin-4-yl]-2-(4-{[(4-methanesulfonyl- |
| | 2-methoxyphenyl)amino]methyl}phenyl)-1-(2,2,2-trifluoroethyl)-1H-indol-4- |
| | amine |
| 2238. | N-[(3R,4S)-3-fluoro-1-methylpiperidin-4-yl]-2-(4-{[(4- |
| | methanesulfonylphenyl)amino]methyl}phenyl)-1-(2,2,2-trifluoroethyl)-1H- |
| | indol-4-amine |
| 2239. | (+/−)-N-{[4-(4-{[(3R,4S)-3-fluoro-1-methylpiperidin-4-yl]amino}-1-(2,2,2- |
| | trifluoroethyl)-1H-indol-2-yl)phenyl]methyl}benzamide |
| 2240. | (+/−)-N-{[4-(4-{[(3R,4S)-3-fluoro-1-methylpiperidin-4-yl]amino}-1-(2,2,2- |
| | trifluoroethyl)-1H-indol-2-yl)phenyl]methyl}cyclopropanecarboxamide |
| 2241. | 1-methoxy-3-(4-{[2-(3-methyl-2H-indazol-6-yl)-1-(2,2,2-trifluoroethyl)-1H- |
| | indol-4-yl]amino}piperidin-1-yl)propan-2-ol |
| 2242. | 1-(4-{[2-(2H-indazol-6-yl)-1-(2,2,2-trifluoroethyl)-1H-indol-4- |
| | yl]amino}piperidin-1-yl)-3-methoxypropan-2-ol |
| 2243. | 4-{[1-(2-hydroxy-3-methoxypropyl)piperidin-4-yl]amino}-1-(2,2,2- |
| | trifluoroethyl)-2′,3′-dihydro-1H,1′H-[2,6′-biindol]-2′-one |
| 2244. | 4-(4-((1-(2-hydroxy-3-methoxypropyl)piperidin-4-yl)amino)-1-(2,2,2- |
| | trifluoroethyl)-1H-indol-2-yl)isoindolin-1-one |
| 2245. | N-[3-(4-{[1-(2-hydroxy-3-methoxypropyl)piperidin-4-yl]amino}-1-(2,2,2- |
| | trifluoroethyl)-1H-indol-2-yl)phenyl]acetamide |

TABLE 3
Heteroaryl-linked indole compounds of the disclosure.

| Mol # | IUPAC name |
|---|---|
| 2246. | 4-((2-(6-methoxypyridin-3-yl)-1-(2,2,2-trifluoroethyl)-1H-indol-4- |
| | yl)amino)tetrahydro-2H-thiopyran 1,1-dioxide-indol-4-amine |
| 2247. | 4-((2-(6-methylpyridin-3-yl)-1-(2,2,2-trifluoroethyl)-1H-indol-4- |
| | yl)amino)tetrahydro-2H-thiopyran 1,1-dioxide |
| 2248. | 4-((2-(6-(dimethylamino)pyridin-3-yl)-1-(2,2,2-trifluoroethyl)-1H-indol-4- |
| | yl)amino)tetrahydro-2H-thiopyran 1,1-dioxide |
| 2249. | 4-((2-(quinolin-4-yl)-1-(2,2,2-trifluoroethyl)-1H-indol-4-yl)amino)tetrahydro- |
| | 2H-thiopyran 1,1-dioxide |
| 2250. | 4-((2-(2-fluoropyridin-3-yl)-1-(2,2,2-trifluoroethyl)-1H-indol-4- |
| | yl)amino)tetrahydro-2H-thiopyran 1,1-dioxide |
| 2251. | 1-(4-((2-(5-aminopyridin-3-yl)-1-(2,2,2-trifluoroethyl)-1H-indol-4- |
| | yl)amino)piperidin-1-yl)-3-methoxypropan-2-ol |
| 2252. | 2-(2-amino-6-phenylpyridin-4-yl)-N-(1-methylpiperidin-4-yl)-1-(2,2,2- |
| | trifluoroethyl)-1H-indol-4-amine |
| 2253. | 5-(4-((1,1-dioxidotetrahydro-2H-thiopyran-4-yl)amino)-1-(2,2,2- |
| | trifluoroethyl)-1H-indol-2-yl)-N-methylpicolinamide |
| 2254. | 2-(2-amino-6-phenylpyrimidin-4-yl)-N-(1-methylpiperidin-4-yl)-1-(2,2,2- |
| | trifluoroethyl)-1H-indol-4-amine |
| 2255. | 2-(2-amino-6-(cyclohex-1-en-1-yl)pyrimidin-4-yl)-N-(1-methylpiperidin-4-yl)- |
| | 1-(2,2,2-trifluoroethyl)-1H-indol-4-amine |
| 2256. | 2-(2-amino-6-cyclohexylpyrimidin-4-yl)-N-(1-methylpiperidin-4-yl)-1-(2,2,2- |
| | trifluoroethyl)-1H-indol-4-amine |
| 2257. | 2-(2-(methylamino)pyrimidin-4-yl)-N-(1-methylpiperidin-4-yl)-1-(2,2,2- |
| | trifluoroethyl)-1H-indol-4-amine |
| 2258. | 2-(2-aminopyrimidin-4-yl)-N-(1-methylpiperidin-4-yl)-1-(2,2,2-trifluoroethyl)- |
| | 1H-indol-4-amine |
| 2259. | 4-{[2-(1-methyl-1H-pyrazol-3-yl)-1-(2,2,2-trifluoroethyl)-1H-indol-4- |
| | yl]amino}-1λ⁶-thiane-1,1-dione |
| 2260. | 4-({2-[1-(2-hydroxyethyl)-1H-pyrazol-4-yl]-1-(2,2,2-trifluoroethyl)-1H-indol- |
| | 4-yl}amino)-1λ⁶-thiane-1,1-dione |
| 2261. | 4-[(2-{1-[(pyridin-3-yl)methyl]-1H-pyrazol-4-yl}-1-(2,2,2-trifluoroethyl)-1H- |
| | indol-4-yl)amino]-1λ⁶-thiane-1,1-dione |
| 2262. | 4-((2-(1-(2-methoxyethyl)-1H-pyrazol-4-yl)-1-(2,2,2-trifluoroethyl)-1H-indol- |
| | 4-yl)amino)tetrahydro-2H-thiopyran 1,1-dioxide |
| 2263. | 4-[(2-{1-[(pyridin-4-yl)methyl]-1H-pyrazol-4-yl}-1-(2,2,2-trifluoroethyl)-1H- |
| | indol-4-yl)amino]-1λ⁶-thiane-1,1-dione |
| 2264. | 2-(5-amino-1-phenyl-1H-pyrazol-3-yl)-N-(1-methylpiperidin-4-yl)-1-(2,2,2- |
| | trifluoroethyl)-1H-indol-4-amine |
| 2265. | 2-{5-[amino(phenyl)methyl]-1H-pyrazol-3-yl}-N-(1-methylpiperidin-4-yl)-1- |
| | (2,2,2-trifluoroethyl)-1H-indol-4-amine |
| 2266. | 2-(5-amino-1H-pyrazol-3-yl)-N-(1-methylpiperidin-4-yl)-1-(2,2,2- |
| | trifluoroethyl)-1H-indol-4-amine |
| 2267. | 2-(5-{[(4-methanesulfonylphenyl)amino]methyl}-1H-pyrazol-3-yl)-N-(1- |
| | methylpiperidin-4-yl)-1-(2,2,2-trifluoroethyl)-1H-indol-4-amine |
| 2268. | 2-[5-(methylamino)-1H-pyrazol-3-yl]-N-(1-methylpiperidin-4-yl)-1-(2,2,2- |
| | trifluoroethyl)-1H-indol-4-amine |
| 2269. | 2-(5-{[(4-methanesulfonylphenyl)amino]methyl}-1,3,4-thiadiazol-2-yl)-N-(1- |
| | methylpiperidin-4-yl)-1-(2,2,2-trifluoroethyl)-1H-indol-4-amine |
| 2270. | N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2- |
| | yl]methyl}cyclopropanecarboxamide |
| 2271. | N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}benzamide |
| 2272. | (+/−)-N-[(3R,4S)-3-fluoro-1-methylpiperidin-4-yl]-2-(5-{[(4-methanesulfonyl- |
| | 2-methoxyphenyl)amino]methyl}-1,3,4-thiadiazol-2-yl)-1-(2,2,2- |
| | trifluoroethyl)-1H-indol-4-amine |
| 2273. | (+/−)-N-[(3R,4R)-3-fluoro-1-methylpiperidin-4-yl]-2-(5-{[(4-methanesulfonyl- |
| | 2-methoxyphenyl)amino]methyl}-1,3,4-thiadiazol-2-yl)-1-(2,2,2- |
| | trifluoroethyl)-1H-indol-4-amine |
| 2274. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2- |
| | yl]methyl}cyclopentanecarboxamide |
| 2275. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}thiophene-2- |
| | carboxamide |
| 2276. | 1-fluoro-N-[(5-{4-[(1-methylpiperidin-4-yl)amino]-1-(2,2,2-trifluoroethyl)-1H- |
| | indol-2-yl}-1,3,4-thiadiazol-2-yl)methyl]cyclopropane-1-carboxamide |
| 2277. | (+/−)-2,2-difluoro-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2278. | (+/−)-(1R,2S)-2-methyl-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2279. | (+/−)-(1R,2R)-2-methyl-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2280. | N-[(5-{4-[(1-methylpiperidin-4-yl)amino]-1-(2,2,2-trifluoroethyl)-1H-indol-2- |
| | yl}-1,3,4-thiadiazol-2-yl)methyl]cyclopropanecarboxamide |
| 2281. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}oxetane-3- |
| | carboxamide |
| 2282. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2- |
| | yl]methyl}cyclobutanecarboxamide |
| 2283. | (+/−)-methyl N-{[5-(4-{[(3R,4S)-3-fluoro-1-methylpiperidin-4-yl]amino}-1- |
| | (2,2,2-trifluoroethyl)-1H-indol-2-yl)-1,3,4-thiadiazol-2-yl]methyl}carbamate |
| 2284. | methyl 4-{[(5-{4-[(1-methylpiperidin-4-yl)amino]-1-(2,2,2-trifluoroethyl)-1H- |
| | indol-2-yl}-1,3,4-thiadiazol-2-yl)methyl]carbamoyl}benzoate |
| 2285. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}-1- |
| | methylpiperidine-4-carboxamide |
| 2286. | N-[(5-{4-[(1-methylpiperidin-4-yl)amino]-1-(2,2,2-trifluoroethyl)-1H-indol-2- |
| | yl}-1,3,4-thiadiazol-2-yl)methyl]pyridine-2-carboxamide |
| 2287. | N-[(5-{4-[(1-methylpiperidin-4-yl)amino]-1-(2,2,2-trifluoroethyl)-1H-indol-2- |
| | yl}-1,3,4-thiadiazol-2-yl)methyl]pyridine-3-carboxamide |
| 2288. | (+/−)-N-[(3R,4S)-3-fluoro-1-methylpiperidin-4-yl]-2-{5- |
| | [(methylamino)methyl]-1,3,4-thiadiazol-2-yl}-1-(2,2,2-trifluoroethyl)-1H- |
| | indol-4-amine |
| 2289. | (+/−)-benzyl N-{[5-(4-{[(3R,4S)-3-fluoro-1-methylpiperidin-4-yl]amino}-1- |
| | (2,2,2-trifluoroethyl)-1H-indol-2-yl)-1,3,4-thiadiazol-2-yl]methyl}carbamate |
| 2290. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-4-[(morpholin-4-yl)methyl]benzamide |
| 2291. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-3-[(morpholin-4-yl)methyl]benzamide |
| 2292. | N-{[5-(4-{[(3S,4R)-3-fluoro-1-methylpiperidin-4-yl]amino}-1-(2,2,2- |
| | trifluoroethyl)-1H-indol-2-yl)-1,3,4-thiadiazol-2- |
| | yl]methyl}cyclopropanecarboxamide |
| 2293. | N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2- |
| | yl]methyl}cyclopropanecarboxamide |
| 2294. | N-[(5-{4-[(1-methylpiperidin-4-yl)amino]-1-(2,2,2-trifluoroethyl)-1H-indol-2- |
| | yl}-1,3,4-thiadiazol-2-yl)methyl]pyridine-4-carboxamide |
| 2295. | 2-fluoro-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2296. | 3-fluoro-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2297. | 4-fluoro-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2298. | (+/−)-(1S,2S)-N-[(5-{4-[(1-methylpiperidin-4-yl)amino]-1-(2,2,2- |
| | trifluoroethyl)-1H-indol-2-yl}-1,3,4-thiadiazol-2-yl)methyl]-2- |
| | phenylcyclopropane-1-carboxamide |
| 2299. | 4-{[(5-{4-[(1-methylpiperidin-4-yl)amino]-1-(2,2,2-trifluoroethyl)-1H-indol-2- |
| | yl}-1,3,4-thiadiazol-2-yl)methyl]carbamoyl}benzoic acid |
| 2300. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-1H-indazole-5-carboxamide |
| 2301. | 3-methyl-1-[(5-{4-[(1-methylpiperidin-4-yl)amino]-1-(2,2,2-trifluoroethyl)- |
| | 1H-indol-2-yl}-1,3,4-thiadiazol-2-yl)methyl]urea |
| 2302. | 2-methyl-N-[(5-{4-[(1-methylpiperidin-4-yl)amino]-1-(2,2,2-trifluoroethyl)- |
| | 1H-indol-2-yl}-1,3,4-thiadiazol-2-yl)methyl]propanamide |
| 2303. | N-[(5-{4-[(1-methylpiperidin-4-yl)amino]-1-(2,2,2-trifluoroethyl)-1H-indol-2- |
| | yl}-1,3,4-thiadiazol-2-yl)methyl]acetamide |
| 2304. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-2-phenylacetamide |
| 2305. | 2-methoxy-N-[(5-{4-[(1-methylpiperidin-4-yl)amino]-1-(2,2,2-trifluoroethyl)- |
| | 1H-indol-2-yl}-1,3,4-thiadiazol-2-yl)methyl]acetamide |
| 2306. | N-[(3R,4S)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)- |
| | 1H-indol-4-amine |
| 2307. | N-[(3S,4R)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)- |
| | 1H-indol-4-amine |
| 2308. | 4-methoxy-N-[(5-{4-[(1-methylpiperidin-4-yl)amino]-1-(2,2,2-trifluoroethyl)- |
| | 1H-indol-2-yl}-1,3,4-thiadiazol-2-yl)methyl]benzamide |
| 2309. | 3-methoxy-N-[(5-{4-[(1-methylpiperidin-4-yl)amino]-1-(2,2,2-trifluoroethyl)- |
| | 1H-indol-2-yl}-1,3,4-thiadiazol-2-yl)methyl]benzamide |
| 2310. | N-[(5-{4-[(1-methylpiperidin-4-yl)amino]-1-(2,2,2-trifluoroethyl)-1H-indol-2- |
| | yl}-1,3,4-thiadiazol-2-yl)methyl]butanamide |
| 2311. | 2-methoxy-N-[(5-{4-[(1-methylpiperidin-4-yl)amino]-1-(2,2,2-trifluoroethyl)- |
| | 1H-indol-2-yl}-1,3,4-thiadiazol-2-yl)methyl]benzamide |
| 2312. | 2-[5-(aminomethyl)-1,3,4-thiadiazol-2-yl]-N-(1-methylpiperidin-4-yl)-1-(2,2,2- |
| | trifluoroethyl)-1H-indol-4-amine |
| 2313. | 3,3-dimethyl-1-[(5-{4-[(1-methylpiperidin-4-yl)amino]-1-(2,2,2- |
| | trifluoroethyl)-1H-indol-2-yl}-1,3,4-thiadiazol-2-yl)methyl]urea |
| 2314. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-1H-indazole-6-carboxamide |
| 2315. | benzyl N-{[5-(4-{[(3S,4R)-3-fluoropiperidin-4-yl]amino}-1-(2,2,2- |
| | trifluoroethyl)-1H-indol-2-yl)-1,3,4-thiadiazol-2-yl]methyl}carbamate |
| 2316. | 1-methyl-N-[(5-{4-[(1-methylpiperidin-4-yl)amino]-1-(2,2,2-trifluoroethyl)- |
| | 1H-indol-2-yl}-1,3,4-thiadiazol-2-yl)methyl]cyclopropane-1-carboxamide |
| 2317. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-1H-pyrazole-4-carboxamide |
| 2318. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-1H-pyrazole-5-carboxamide |
| 2319. | 1-ethyl-N-[(5-{4-[(1-methylpiperidin-4-yl)amino]-1-(2,2,2-trifluoroethyl)-1H- |
| | indol-2-yl}-1,3,4-thiadiazol-2-yl)methyl]cyclopropane-1-carboxamide |
| 2320. | (+/−)-methyl(1R,2R)-2-{[(5-{4-[(1-methylpiperidin-4-yl)amino]-1-(2,2,2- |
| | trifluoroethyl)-1H-indol-2-yl}-1,3,4-thiadiazol-2- |
| | yl)methyl]carbamoyl}cyclopropane-1-carboxylate |
| 2321. | (+/−)-(1R,2R)-2-{[(5-{4-[(1-methylpiperidin-4-yl)amino]-1-(2,2,2- |
| | trifluoroethyl)-1H-indol-2-yl}-1,3,4-thiadiazol-2- |
| | yl)methyl]carbamoyl}cyclopropane-1-carboxylic acid |
| 2322. | 1-methyl-N-[(5-{4-[(1-methylpiperidin-4-yl)amino]-1-(2,2,2-trifluoroethyl)- |
| | 1H-indol-2-yl}-1,3,4-thiadiazol-2-yl)methyl]-1H-pyrrole-3-carboxamide |
| 2323. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-1H-pyrrole-3-carboxamide |
| 2324. | N-[(5-{4-[(1-methylpiperidin-4-yl)amino]-1-(2,2,2-trifluoroethyl)-1H-indol-2- |
| | yl}-1,3,4-thiadiazol-2-yl)methyl]-2-(thiophen-2-yl)cyclopropane-1- |
| | carboxamide |
| 2325. | N-(1-methylpiperidin-4-yl)-2-(5-{[(pyrrolidin-3-yl)amino]methyl}-1,3,4- |
| | thiadiazol-2-yl)-1-(2,2,2-trifluoroethyl)-1H-indol-4-amine |
| 2326. | (+/−)-(1R,2S)-2-{[(5-{4-[(1-methylpiperidin-4-yl)amino]-1-(2,2,2- |
| | trifluoroethyl)-1H-indol-2-yl}-1,3,4-thiadiazol-2- |
| | yl)methyl]carbamoyl}cyclopropane-1-carboxylic acid |
| 2327. | N-(1-methylpiperidin-4-yl)-2-(5-{[(1H-pyrazol-4-yl)amino]methyl}-1,3,4- |
| | thiadiazol-2-yl)-1-(2,2,2-trifluoroethyl)-1H-indol-4-amine |
| 2328. | N-((5-(4-(((3S,4R)-3-fluoro-1-methylpiperidin-4-yl)amino)-1-(2,2,2- |
| | trifluoroethyl)-1H-indol-2-yl)-1,3,4-thiadiazol-2- |
| | yl)methyl)cyclopentanecarboxamide |
| 2329. | N-((3S,4R)-3-fluoro-1-methylpiperidin-4-yl)-2-(5-(((2-methoxy-4- |
| | (methylsulfonyl)phenyl)amino)methyl)-1,3,4-thiadiazol-2-yl)-1-(2,2,2- |
| | trifluoroethyl)-1H-indol-4-amine |
| 2330. | N-((5-(4-(((3S,4R)-3-fluoro-1-methylpiperidin-4-yl)amino)-1-(2,2,2- |
| | trifluoroethyl)-1H-indol-2-yl)-1,3,4-thiadiazol-2-yl)methyl)benzamide |
| 2331. | N-((5-(4-(((3S,4R)-3-fluoro-1-methylpiperidin-4-yl)amino)-1-(2,2,2- |
| | trifluoroethyl)-1H-indol-2-yl)-1,3,4-thiadiazol-2- |
| | yl)methyl)cyclopropanecarboxamide |
| 2332. | 2-(5-((dimethylamino)methyl)-1,3,4-thiadiazol-2-yl)-N-((3S,4R)-3-fluoro-1- |
| | methylpiperidin-4-yl)-1-(2,2,2-trifluoroethyl)-1H-indol-4-amine |
| 2333. | N-((3S,4S)-3-fluoro-1-methylpiperidin-4-yl)-2-(5-(((2-methoxy-4- |
| | (methylsulfonyl)phenyl)amino)methyl)-1,3,4-thiadiazol-2-yl)-1-(2,2,2- |
| | trifluoroethyl)-1H-indol-4-amine |
| 2334. | N-{[2-(4-{[(3S,4R)-3-fluoro-1-methylpiperidin-4-yl]amino}-1-(2,2,2- |
| | trifluoroethyl)-1H-indol-2-yl)-1,3-thiazol-4- |
| | yl]methyl}cyclopropanecarboxamide |
| 2335. | N-{[2-(4-{[(3S,4R)-3-fluoro-1-methylpiperidin-4-yl]amino}-1-(2,2,2- |
| | trifluoroethyl)-1H-indol-2-yl)-1,3-thiazol-4-yl]methyl}benzamide |
| 2336. | (+/−)-N-{[2-(4-{[(3R,4S)-3-fluoro-1-methylpiperidin-4-yl]amino}-1-(2,2,2- |
| | trifluoroethyl)-1H-indol-2-yl)-1,3-thiazol-5-yl]methyl}benzamide |
| 2337. | N-[(2-{4-[(1-methylpiperidin-4-yl)amino]-1-(2,2,2-trifluoroethyl)-1H-indol-2- |
| | yl}-1,3-thiazol-5-yl)methyl]benzamide |
| 2338. | (+/−)-N-[(3R,4S)-3-fluoro-1-methylpiperidin-4-yl]-2-(1,3-thiazol-2-yl)-1- |
| | (2,2,2-trifluoroethyl)-1H-indol-4-amine |
| 2339. | N-[(2-{4-[(1-methylpiperidin-4-yl)amino]-1-(2,2,2-trifluoroethyl)-1H-indol-2- |
| | yl}-1,3-thiazol-5-yl)methyl]cyclopropanecarboxamide |
| 2340. | (+/−)-N-{[2-(4-{[(3R,4S)-3-fluoro-1-methylpiperidin-4-yl]amino}-1-(2,2,2- |
| | trifluoroethyl)-1H-indol-2-yl)-1,3-thiazol-5- |
| | yl]methyl}cyclopropanecarboxamide |
| 2341. | (+/−)-2-[5-(aminomethyl)-1,3-thiazol-2-yl]-N-[(3R,4S)-3-fluoro-1- |
| | methylpiperidin-4-yl]-1-(2,2,2-trifluoroethyl)-1H-indol-4-amine |
| 2342. | 2-(4-(aminomethyl)thiazol-2-yl)-N-((3S,4R)-3-fluoro-1-methylpiperidin-4-yl)- |
| | 1-(2,2,2-trifluoroethyl)-1H-indol-4-amine |
| 2343. | (+/−)-N-[(3R,4S)-3-fluoro-1-methylpiperidin-4-yl]-2-{5- |
| | [(phenylamino)methyl]-1,3-thiazol-2-yl}-1-(2,2,2-trifluoroethyl)-1H-indol-4- |
| | amine |
| 2344. | N-[(3S,4R)-3-fluoro-1-methylpiperidin-4-yl]-2-(5-{[(4-methanesulfonyl-2- |
| | methoxyphenyl)amino]methyl}-1,3-thiazol-2-yl)-1-(2,2,2-trifluoroethyl)-1H- |
| | indol-4-amine |
| 2345. | 2-(5-{[(4-methanesulfonylphenyl)amino]methyl}thiophen-2-yl)-N-(1- |
| | methylpiperidin-4-yl)-1-(2,2,2-trifluoroethyl)-1H-indol-4-amine |
| 2346. | (+/−)-N-[(3R,4S)-3-fluoro-1-methylpiperidin-4-yl]-2-(5-{[(4-methanesulfonyl- |
| | 2-methoxyphenyl)amino]methyl}thiophen-2-yl)-1-(2,2,2-trifluoroethyl)-1H- |
| | indol-4-amine |
| 2347. | (+/−)-N-{[5-(4-{[(3R,4S)-3-fluoro-1-methylpiperidin-4-yl]amino}-1-(2,2,2- |
| | trifluoroethyl)-1H-indol-2-yl)thiophen-2-yl]methyl}benzamide |
| 2348. | (+/−)-N-{[5-(4-{[(3R,4S)-3-fluoro-1-methylpiperidin-4-yl]amino}-1-(2,2,2- |
| | trifluoroethyl)-1H-indol-2-yl)thiophen-2-yl]methyl}cyclopropanecarboxamide |
| 2349. | N-(1-methylpiperidin-4-yl)-2-(5-(((4- |
| | (methylsulfonyl)phenyl)amino)methyl)thiophen-2-yl)-1-(2,2,2-trifluoroethyl)- |
| | 1H-indol-4-amine |
| 2350. | N-((5-(4-(((3S,4R)-3-fluoro-1-methylpiperidin-4-yl)amino)-1-(2,2,2- |
| | trifluoroethyl)-1H-indol-2-yl)thiophen-2-yl)methyl)benzamide |
| 2351. | N-((3S,4R)-3-fluoro-1-methylpiperidin-4-yl)-2-(5-(((2-methoxy-4- |
| | (methylsulfonyl)phenyl)amino)methyl)thiophen-2-yl)-1-(2,2,2-trifluoroethyl)- |
| | 1H-indol-4-amine |
| 2352. | 2-(5-(amino(cyclohexyl)methyl)-1,3,4-oxadiazol-2-yl)-N-(1-methylpiperidin-4- |
| | yl)-1-(2,2,2-trifluoroethyl)-1H-indol-4-amine |
| 2353. | N-(1-methylpiperidin-4-yl)-2-{5-[(phenylamino)methyl]-1,3,4-oxadiazol-2- |
| | yl}-1-(2,2,2-trifluoroethyl)-1H-indol-4-amine |
| 2354. | 2-(5-(amino(tetrahydro-2H-pyran-4-yl)methyl)-1,3,4-oxadiazol-2-yl)-N-(1- |
| | methylpiperidin-4-yl)-1-(2,2,2-trifluoroethyl)-1H-indol-4-amine |
| 2355. | N-[(5-{4-[(1-methylpiperidin-4-yl)amino]-1-(2,2,2-trifluoroethyl)-1H-indol-2- |
| | yl}-1,3,4-oxadiazol-2-yl)methyl]cyclopropanecarboxamide |
| 2356. | 2-(5-{[(4-methanesulfonylphenyl)amino]methyl}-1,3,4-oxadiazol-2-yl)-N-(1- |
| | methylpiperidin-4-yl)-1-(2,2,2-trifluoroethyl)-1H-indol-4-amine |
| 2357. | N-(1-methylpiperidin-4-yl)-2-(5-{[(1H-pyrazol-4-yl)amino]methyl}-1,3,4- |
| | oxadiazol-2-yl)-1-(2,2,2-trifluoroethyl)-1H-indol-4-amine |
| 2358. | 2-(5-(aminomethyl)-1,3,4-oxadiazol-2-yl)-N-(1-methylpiperidin-4-yl)-1-(2,2,2- |
| | trifluoroethyl)-1H-indol-4-amine |
| 2359. | (+/−)-N-[(3R,4S)-3-fluoro-1-methylpiperidin-4-yl]-2-(5-{[(4-methanesulfonyl- |
| | 2-methoxyphenyl)amino]methyl}-1,3,4-oxadiazol-2-yl)-1-(2,2,2- |
| | trifluoroethyl)-1H-indol-4-amine |
| 2360. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-2- |
| | methoxybenzamide |
| 2361. | (+/−)-2-[5-(aminomethyl)-1,3,4-oxadiazol-2-yl]-N-[(3R,4S)-3-fluoro-1- |
| | methylpiperidin-4-yl]-1-(2,2,2-trifluoroethyl)-1H-indol-4-amine |
| 2362. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-4- |
| | methoxybenzamide |
| 2363. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}benzamide |
| 2364. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-3- |
| | methoxybenzamide |
| 2365. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}thiophene-2- |
| | carboxamide |
| 2366. | (+/−)-2-(5-{[(cyclopropylmethyl)amino]methyl}-1,3,4-oxadiazol-2-yl)-N- |
| | [(3R,4S)-3-fluoro-1-methylpiperidin-4-yl]-1-(2,2,2-trifluoroethyl)-1H-indol-4- |
| | amine |
| 2367. | (+/−)-N-[(3R,4S)-3-fluoro-1-methylpiperidin-4-yl]-2-(5-{[(3- |
| | methanesulfonylphenyl)amino]methyl}-1,3,4-oxadiazol-2-yl)-1-(2,2,2- |
| | trifluoroethyl)-1H-indol-4-amine |
| 2368. | (+/−)-N-[(3R,4R)-3-fluoro-1-methylpiperidin-4-yl]-2-(5-{[(4-methanesulfonyl- |
| | 2-methoxyphenyl)amino]methyl}-1,3,4-oxadiazol-2-yl)-1-(2,2,2- |
| | trifluoroethyl)-1H-indol-4-amine |
| 2369. | (+/−)-2-(5-((bis(cyclopropylmethyl)amino)methyl)-1,3,4-oxadiazol-2-yl)-N- |
| | ((3R,4S)-3-fluoro-1-methylpiperidin-4-yl)-1-(2,2,2-trifluoroethyl)-1H-indol-4- |
| | amine |
| 2370. | (+/−)-N-((3S,4R)-3-fluoro-1-methylpiperidin-4-yl)-2-(5-(((3- |
| | (methylsulfonyl)phenyl)amino)methyl)-1,3,4-oxadiazol-2-yl)-1-(2,2,2- |
| | trifluoroethyl)-1H-indol-4-amine |
| 2371. | (+/−)-N-((3S,4R)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2- |
| | trifluoroethyl)-1H-indol-4-amine |
| 2372. | (+/−)-2-(5-(aminomethyl)-1,3,4-oxadiazol-2-yl)-N-((3S,4R)-3-fluoro-1- |
| | methylpiperidin-4-yl)-1-(2,2,2-trifluoroethyl)-1H-indol-4-amine |
| 2373. | (+/−)-N-((3S,4S)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2- |
| | trifluoroethyl)-1H-indol-4-amine |
| 2374. | N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)benzamide |
| 2375. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)thiophene-2- |
| | carboxamide |
| 2376. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-2- |
| | methoxybenzamide |
| 2377. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-3- |
| | methoxybenzamide |
| 2378. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-4- |
| | methoxybenzamide |
| 2379. | (+/−)-2-(5-(((cyclopropylmethyl)amino)methyl)-1,3,4-oxadiazol-2-yl)-N- |
| | ((3S,4R)-3-fluoro-1-methylpiperidin-4-yl)-1-(2,2,2-trifluoroethyl)-1H-indol-4- |
| | amine |
| 2380. | (+/−)-N-((3R,4R)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2- |
| | trifluoroethyl)-1H-indol-4-amine |
| 2381. | 2-(3-{[(4-methanesulfonylphenyl)amino]methyl}-1,2,4-oxadiazol-5-yl)-N-(1- |
| | methylpiperidin-4-yl)-1-(2,2,2-trifluoroethyl)-1H-indol-4-amine |
| 2382. | N-[(5-{4-[(1-methylpiperidin-4-yl)amino]-1-(2,2,2-trifluoroethyl)-1H-indol-2- |
| | yl}-1,2,4-oxadiazol-3-yl)methyl]cyclopropanecarboxamide |
| 2383. | N-[(5-{4-[(1-methylpiperidin-4-yl)amino]-1-(2,2,2-trifluoroethyl)-1H-indol-2- |
| | yl}-1,2,4-oxadiazol-3-yl)methyl]benzamide |
| 2384. | N-[(5-{4-[(1-methylpiperidin-4-yl)amino]-1-(2,2,2-trifluoroethyl)-1H-indol-2- |
| | yl}-1,2,4-oxadiazol-3-yl)methyl]thiophene-2-carboxamide |
| 2385. | 1-methyl-N-[(5-{4-[(1-methylpiperidin-4-yl)amino]-1-(2,2,2-trifluoroethyl)- |
| | 1H-indol-2-yl}-1,2,4-oxadiazol-3-yl)methyl]-1H-pyrazole-4-carboxamide |
| 2386. | 1-methyl-N-[(5-{4-[(1-methylpiperidin-4-yl)amino]-1-(2,2,2-trifluoroethyl)- |
| | 1H-indol-2-yl}-1,2,4-oxadiazol-3-yl)methyl]-1H-pyrazole-3-carboxamide |
| 2387. | N-(1-methylpiperidin-4-yl)-2-{5-[(phenylamino)methyl]-1,2,4-oxadiazol-3- |
| | yl}-1-(2,2,2-trifluoroethyl)-1H-indol-4-amine |
| 2388. | N-[(3-{4-[(1-methylpiperidin-4-yl)amino]-1-(2,2,2-trifluoroethyl)-1H-indol-2- |
| | yl}-1,2,4-oxadiazol-5-yl)methyl]thiophene-2-carboxamide |
| 2389. | N-[(3-{4-[(1-methylpiperidin-4-yl)amino]-1-(2,2,2-trifluoroethyl)-1H-indol-2- |
| | yl}-1,2,4-oxadiazol-5-yl)methyl]benzamide |
| 2390. | (+/−)-(1S,2R)-2-fluoro-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1- |
| | carboxamide |
| 2391. | N-[(3-{4-[(1-methylpiperidin-4-yl)amino]-1-(2,2,2-trifluoroethyl)-1H-indol-2- |
| | yl}-1,2,4-oxadiazol-5-yl)methyl]cyclopropanecarboxamide |
| 2392. | (+/−)-(1S,2S)-2-fluoro-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1- |
| | carboxamide |
| 2393. | 4-chloro-N-[(3-{4-[(1-methylpiperidin-4-yl)amino]-1-(2,2,2-trifluoroethyl)- |
| | 1H-indol-2-yl}-1,2,4-oxadiazol-5-yl)methyl]benzamide |
| 2394. | N-[(3-{4-[(1-methylpiperidin-4-yl)amino]-1-(2,2,2-trifluoroethyl)-1H-indol-2- |
| | yl}-1,2,4-oxadiazol-5-yl)methyl]-1,3-thiazole-2-carboxamide |
| 2395. | 4-fluoro-N-[(3-{4-[(1-methylpiperidin-4-yl)amino]-1-(2,2,2-trifluoroethyl)-1H- |
| | indol-2-yl}-1,2,4-oxadiazol-5-yl)methyl]benzamide |
| 2396. | 4-cyano-N-[(3-{4-[(1-methylpiperidin-4-yl)amino]-1-(2,2,2-trifluoroethyl)-1H- |
| | indol-2-yl}-1,2,4-oxadiazol-5-yl)methyl]benzamide |
| 2397. | 1-methyl-N-[(3-{4-[(1-methylpiperidin-4-yl)amino]-1-(2,2,2-trifluoroethyl)- |
| | 1H-indol-2-yl}-1,2,4-oxadiazol-5-yl)methyl]-1H-pyrrole-3-carboxamide |
| 2398. | 3-[(3-{4-[(1-methylpiperidin-4-yl)amino]-1-(2,2,2-trifluoroethyl)-1H-indol-2- |
| | yl}-1,2,4-oxadiazol-5-yl)methyl]-1-phenylurea |
| 2399. | 1-methyl-N-[(3-{4-[(1-methylpiperidin-4-yl)amino]-1-(2,2,2-trifluoroethyl)- |
| | 1H-indol-2-yl}-1,2,4-oxadiazol-5-yl)methyl]-1H-pyrazole-4-carboxamide |
| 2400. | 1-methyl-N-[(3-{4-[(1-methylpiperidin-4-yl)amino]-1-(2,2,2-trifluoroethyl)- |
| | 1H-indol-2-yl}-1,2,4-oxadiazol-5-yl)methyl]-1H-pyrazole-3-carboxamide |
| 2401. | (+/−)-(1R,2R)-N-[(3-{4-[(1-methylpiperidin-4-yl)amino]-1-(2,2,2- |
| | trifluoroethyl)-1H-indol-2-yl}-1,2,4-oxadiazol-5-yl)methyl]-2- |
| | phenylcyclopropane-1-carboxamide |
| 2402. | (+/−)-(1R,2R)-2-methyl-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1- |
| | carboxamide |
| 2403. | (+/−)-(1R,2S)-2-methyl-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1- |
| | carboxamide |
| 2404. | N-[(3-{4-[(oxan-4-yl)amino]-1-(2,2,2-trifluoroethyl)-1H-indol-2-yl}-1,2,4- |
| | oxadiazol-5-yl)methyl]cyclopropanecarboxamide |
| 2405. | N-({3-[4-(benzylamino)-1-(2,2,2-trifluoroethyl)-1H-indol-2-yl]-1,2,4- |
| | oxadiazol-5-yl}methyl)cyclopropanecarboxamide |
| 2406. | N-[(3-{4-[(1-acetylpiperidin-4-yl)amino]-1-(2,2,2-trifluoroethyl)-1H-indol-2- |
| | yl}-1,2,4-oxadiazol-5-yl)methyl]cyclopropanecarboxamide |
| 2407. | N-[(3-{4-[(1,1-dioxo-1λ⁶-thian-4-yl)amino]-1-(2,2,2-trifluoroethyl)-1H-indol- |
| | 2-yl}-1,2,4-oxadiazol-5-yl)methyl]cyclopropanecarboxamide |
| 2408. | N-[(3-{4-[(1-benzylpiperidin-4-yl)amino]-1-(2,2,2-trifluoroethyl)-1H-indol-2- |
| | yl}-1,2,4-oxadiazol-5-yl)methyl]cyclopropanecarboxamide |
| 2409. | N-[(3-{4-[(1-cyclopropylpiperidin-4-yl)amino]-1-(2,2,2-trifluoroethyl)-1H- |
| | indol-2-yl}-1,2,4-oxadiazol-5-yl)methyl]cyclopropanecarboxamide |
| 2410. | N-[(3-{4-[(cyclopropylmethyl)amino]-1-(2,2,2-trifluoroethyl)-1H-indol-2-yl}- |
| | 1,2,4-oxadiazol-5-yl)methyl]cyclopropanecarboxamide |
| 2411. | N-[(3-{4-[(cyclobutylmethyl)amino]-1-(2,2,2-trifluoroethyl)-1H-indol-2-yl}- |
| | 1,2,4-oxadiazol-5-yl)methyl]cyclopropanecarboxamide |
| 2412. | (+/−)-N-[(3-{4-[(pyrrolidin-3-yl)amino]-1-(2,2,2-trifluoroethyl)-1H-indol-2- |
| | yl}-1,2,4-oxadiazol-5-yl)methyl]cyclopropanecarboxamide |
| 2413. | N-[(3-{4-[(1-ethylpiperidin-4-yl)amino]-1-(2,2,2-trifluoroethyl)-1H-indol-2- |
| | yl}-1,2,4-oxadiazol-5-yl)methyl]cyclopropanecarboxamide |
| 2414. | (+/−)-N-[(3-{4-[(1-methylpyrrolidin-3-yl)amino]-1-(2,2,2-trifluoroethyl)-1H- |
| | indol-2-yl}-1,2,4-oxadiazol-5-yl)methyl]cyclopropanecarboxamide |
| 2415. | N-{[3-(4-{[(425zetidine-3-yl)methyl]amino}-1-(2,2,2-trifluoroethyl)-1H-indol- |
| | 2-yl)-1,2,4-oxadiazol-5-yl]methyl}cyclopropanecarboxamide |
| 2416. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5- |
| | yl]methyl}cyclopropanecarboxamide |
| 2417. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}benzamide |
| 2418. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5- |
| | yl]methyl}cyclopropanecarboxamide |
| 2419. | N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5- |
| | yl]methyl}cyclopropanecarboxamide |
| 2420. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-2- |
| | carboxamide |
| 2421. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3- |
| | carboxamide |
| 2422. | (+/−)-N-[(3R,4S)-3-fluoro-1-methylpiperidin-4-yl]-2-(5-{[(4- |
| | methanesulfonylphenyl)amino]methyl}-1,2,4-oxadiazol-3-yl)-1-(2,2,2- |
| | trifluoroethyl)-1H-indol-4-amine |
| 2423. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1,3-thiazole-5- |
| | carboxamide |
| 2424. | (+/−)-1-ethyl-N-{[3-(4-{[(3R,4S)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole- |
| | 3-carboxamide |
| 2425. | (+/−)-(1R,2R)-N-{[3-(4-{[(3R,4S)-3-fluoro-1-methylpiperidin-4-yl]amino}-1- |
| | (2,2,2-trifluoroethyl)-1H-indol-2-yl)-1,2,4-oxadiazol-5-yl]methyl}-2- |
| | phenylcyclopropane-1-carboxamide |
| 2426. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-methyl-1H- |
| | pyrrole-3-carboxamide |
| 2427. | N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1-methyl-1H- |
| | pyrrole-3-carboxamide |
| 2428. | (+/−)-N-[(3R,4S)-3-fluoro-1-methylpiperidin-4-yl]-2-(5-{[(4-methanesulfonyl- |
| | 2-methoxyphenyl)amino]methyl}-1,2,4-oxadiazol-3-yl)-1-(2,2,2- |
| | trifluoroethyl)-1H-indol-4-amine |
| 2429. | (+/−)-1-tert-butyl-N-{[3-(4-{[(3R,4S)-3-fluoro-1-methylpiperidin-4-yl]amino}- |
| | 1-(2,2,2-trifluoroethyl)-1H-indol-2-yl)-1,2,4-oxadiazol-5-yl]methyl}-1H- |
| | pyrrole-3-carboxamide |
| 2430. | (+/−)-(1R,2R)-N-{[3-(4-{[(3RS,4SR)-3-fluoro-1-methylpiperidin-4-yl]amino}- |
| | 1-(2,2,2-trifluoroethyl)-1H-indol-2-yl)-1,2,4-oxadiazol-5-yl]methyl}-2- |
| | phenylcyclopropane-1-carboxamide |
| 2431. | 1-ethyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2432. | 1-ethyl-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2433. | (+/−)-N-[(3R,4S)-3-fluoropiperidin-4-yl]-2-(5-{[(4-methanesulfonyl-2- |
| | methoxyphenyl)amino]methyl}-1,2,4-oxadiazol-3-yl)-1-(2,2,2-trifluoroethyl)- |
| | 1H-indol-4-amine |
| 2434. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3- |
| | carboxamide |
| 2435. | N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3- |
| | carboxamide |
| 2436. | (+/−)-N-[(3R,4S)-3-fluoropiperidin-4-yl]-2-(5-{[methyl(1H-pyrazol-4- |
| | yl)amino]methyl}-1,2,4-oxadiazol-3-yl)-1-(2,2,2-trifluoroethyl)-1H-indol-4- |
| | amine |
| 2437. | (+/−)-N-[(3R,4S)-3-fluoro-1-methylpiperidin-4-yl]-2-(5-{[methyl(1H-pyrazol- |
| | 4-yl)amino]methyl}-1,2,4-oxadiazol-3-yl)-1-(2,2,2-trifluoroethyl)-1H-indol-4- |
| | amine |
| 2438. | (1RS,2RS)-2-cyano-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}cyclopropane-1-carboxamide |
| 2439. | 1-tert-butyl-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole- |
| | 3-carboxamide |
| 2440. | (1RS,2SR)-2-cyano-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}cyclopropane-1-carboxamide |
| 2441. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}indolizine-2- |
| | carboxamide |
| 2442. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-phenyl-1H- |
| | imidazole-4-carboxamide |
| 2443. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2- |
| | methoxyethyl)-1H-pyrrole-3-carboxamide |
| 2444. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(propan-2-yl)- |
| | 1H-pyrrole-3-carboxamide |
| 2445. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}pyridine-4- |
| | carboxamide |
| 2446. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}pyridine-2- |
| | carboxamide |
| 2447. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}pyridine-3- |
| | carboxamide |
| 2448. | N-[(3S,4R)-3-fluoropiperidin-4-yl]-2-(5-{[(1H-pyrazol-4-yl)amino]methyl}- |
| | 1,2,4-oxadiazol-3-yl)-1-(2,2,2-trifluoroethyl)-1H-indol-4-amine |
| 2449. | benzyl N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamate |
| 2450. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2-fluoroethyl)- |
| | 1H-pyrrole-3-carboxamide |
| 2451. | (1S,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2- |
| | phenylcyclopropane-1-carboxamide |
| 2452. | (1R,2S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2- |
| | phenylcyclopropane-1-carboxamide |
| 2453. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-5- |
| | methylthiophene-3-carboxamide |
| 2454. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-4- |
| | methylthiophene-3-carboxamide |
| 2455. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(4- |
| | fluorophenyl)cyclopropane-1-carboxamide |
| 2456. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2- |
| | methylthiophene-3-carboxamide |
| 2457. | (1s,3r)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3- |
| | methylcyclobutane-1-carboxamide |
| 2458. | 5-chloro-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3- |
| | carboxamide |
| 2459. | N-[(3S,4R)-3-fluoropiperidin-4-yl]-2-(5-{[(1H-pyrazol-5-yl)amino]methyl}- |
| | 1,2,4-oxadiazol-3-yl)-1-(2,2,2-trifluoroethyl)-1H-indol-4-amine |
| 2460. | 2-chloro-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3- |
| | carboxamide |
| 2461. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}pyrazolo[1,5- |
| | a]pyridine-2-carboxamide |
| 2462. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}imidazo[1,2- |
| | a]pyridine-2-carboxamide |
| 2463. | 1-cyclopropyl-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole- |
| | 3-carboxamide |
| 2464. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1,5-dimethyl-1H- |
| | pyrrole-3-carboxamide |
| 2465. | 4-(dimethylamino)-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}benzamide |
| 2466. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5- |
| | yl]methyl}benzenesulfonamide |
| 2467. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5- |
| | yl]methyl}cyclobutanecarboxamide |
| 2468. | (1r,3s)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3- |
| | methylcyclobutane-1-carboxamide |
| 2469. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(2- |
| | fluorophenyl)cyclopropane-1-carboxamide |
| 2470. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(1-methyl-1H- |
| | pyrazol-4-yl)cyclopropane-1-carboxamide |
| 2471. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(pyridin-2- |
| | yl)cyclopropane-1-carboxamide |
| 2472. | N-[(3S,4R)-3-fluoro-1-methylpiperidin-4-yl]-2-(5-{[(1H-pyrazol-4- |
| | yl)amino]methyl}-1,2,4-oxadiazol-3-yl)-1-(2,2,2-trifluoroethyl)-1H-indol-4- |
| | amine |
| 2473. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}acetamide |
| 2474. | 1-[2-(dimethylamino)ethyl]-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}-1H-pyrrole-3-carboxamide |
| 2475. | N-[(3S,4R)-3-fluoro-1-methylpiperidin-4-yl]-2-(5-{[(1H-pyrazol-5- |
| | yl)amino]methyl}-1,2,4-oxadiazol-3-yl)-1-(2,2,2-trifluoroethyl)-1H-indol-4- |
| | amine |
| 2476. | 1-tert-butyl-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H- |
| | pyrazole-4-carboxamide |
| 2477. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(propan-2-yl)- |
| | 1H-imidazole-4-carboxamide |
| 2478. | (1S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2,2- |
| | dimethylcyclopropane-1-carboxamide |
| 2479. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(4- |
| | methylpiperazin-1-yl)methyl]cyclopropane-1-carboxamide |
| 2480. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(3- |
| | fluorophenyl)cyclopropane-1-carboxamide |
| 2481. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-methyl-1H- |
| | 1,2,3-triazole-4-carboxamide |
| 2482. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(1- |
| | hydroxypropan-2-yl)-1H-pyrrole-3-carboxamide |
| 2483. | 2-[3-({[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamoyl)-1H- |
| | pyrrol-1-yl]acetic acid |
| 2484. | (1R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2,2- |
| | dimethylcyclopropane-1-carboxamide |
| 2485. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2- |
| | methylpropyl)-1H-pyrrole-3-carboxamide |
| 2486. | 1-(cyclopropylmethyl)-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}-1H-pyrrole-3-carboxamide |
| 2487. | 3-(dimethylamino)-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}benzamide |
| 2488. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-4-(pyrrolidin-1- |
| | yl)benzamide |
| 2489. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2- |
| | hydroxypropyl)-1H-pyrrole-3-carboxamide |
| 2490. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(morpholin-4- |
| | yl)methyl]cyclopropane-1-carboxamide |
| 2491. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-{[(propan-2- |
| | yl)amino]methyl}cyclopropane-1-carboxamide |
| 2492. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2- |
| | [(propylamino)methyl]cyclopropane-1-carboxamide |
| 2493. | 3-[3-({[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamoyl)-1H- |
| | pyrrol-1-yl]propanoic acid |
| 2494. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2- |
| | hydroxyethyl)-1H-pyrrole-3-carboxamide |
| 2495. | 1-(2,2-difluoroethyl)-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}-1H-pyrrole-3-carboxamide |
| 2496. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2-methoxy-2- |
| | methylpropyl)-1H-pyrrole-3-carboxamide |
| 2497. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2-hydroxy-2- |
| | methylpropyl)-1H-pyrrole-3-carboxamide |
| 2498. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2- |
| | methoxypropyl)-1H-pyrrole-3-carboxamide |
| 2499. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-methyl-1H- |
| | indole-6-carboxamide |
| 2500. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(propan-2-yl)- |
| | 1H-pyrazole-4-carboxamide |
| 2501. | 4-(4,4-difluoropiperidin-1-yl)-N-{[3-(4-{[(3S,4R)-3-fluoro-1-methylpiperidin- |
| | 4-yl]amino}-1-(2,2,2-trifluoroethyl)-1H-indol-2-yl)-1,2,4-oxadiazol-5- |
| | yl]methyl}benzamide |
| 2502. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(propan-2-yl)- |
| | 1H-imidazole-5-carboxamide |
| 2503. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-indole-5- |
| | carboxamide |
| 2504. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-methyl-1H- |
| | indole-5-carboxamide |
| 2505. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-4-(morpholin-4- |
| | yl)benzamide |
| 2506. | 2-[5-(aminomethyl)-1,2,4-oxadiazol-3-yl]-N-[(3S,4R)-3-fluoro-1- |
| | methylpiperidin-4-yl]-1-(2,2,2-trifluoroethyl)-1H-indol-4-amine |
| 2507. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-(morpholin-4- |
| | yl)benzamide |
| 2508. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-(pyrrolidin-1- |
| | yl)benzamide |
| 2509. | (1R,2R)-2-[(dimethylamino)methyl]-N-{[3-(4-{[(3S,4R)-3-fluoro-1- |
| | methylpiperidin-4-yl]amino}-1-(2,2,2-trifluoroethyl)-1H-indol-2-yl)-1,2,4- |
| | oxadiazol-5-yl]methyl}cyclopropane-1-carboxamide |
| 2510. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(pyrrolidin-1- |
| | yl)methyl]cyclopropane-1-carboxamide |
| 2511. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-methyl-1H- |
| | indole-5-carboxamide |
| 2512. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-indole-6- |
| | carboxamide |
| 2513. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1-(2- |
| | methoxyethyl)-1H-imidazole-5-carboxamide |
| 2514. | (1R,2R)-2-ethyl-N-{[3-(4-{[(3S,4R)-3-fluoro-1-methylpiperidin-4-yl]amino}- |
| | 1-(2,2,2-trifluoroethyl)-1H-indol-2-yl)-1,2,4-oxadiazol-5- |
| | yl]methyl}cyclopropane-1-carboxamide |
| 2515. | 1-tert-butyl-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H- |
| | imidazole-5-carboxamide |
| 2516. | (1S,2S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-{[(propan-2- |
| | yl)amino]methyl}cyclopropane-1-carboxamide |
| 2517. | N-{[3-(4-{[(3S,4R)-1-ethyl-3-fluoropiperidin-4-yl]amino}-1-(2,2,2- |
| | trifluoroethyl)-1H-indol-2-yl)-1,2,4-oxadiazol-5-yl]methyl}-1-(2- |
| | methoxyethyl)-1H-pyrrole-3-carboxamide |
| 2518. | N-{[3-(4-{[(3R,4S)-1-ethyl-3-fluoropiperidin-4-yl]amino}-1-(2,2,2- |
| | trifluoroethyl)-1H-indol-2-yl)-1,2,4-oxadiazol-5-yl]methyl}-1-(2- |
| | methoxyethyl)-1H-pyrrole-3-carboxamide |
| 2519. | N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-methyl-1H- |
| | indole-6-carboxamide |
| 2520. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3- |
| | yl]methyl}cyclopropanecarboxamide |
| 2521. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}benzamide |
| 2522. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}thiophene-2- |
| | carboxamide |
| 2523. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}thiophene-3- |
| | carboxamide |
| 2524. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}-1,3-thiazole-5- |
| | carboxamide |
| 2525. | N-((3-(4-(((3S,4R)-3-fluoro-1-methylpiperidin-4-yl)amino)-1-(2,2,2- |
| | trifluoroethyl)-1H-indol-2-yl)-1,2,4-oxadiazol-5-yl)methyl)-1-(1-fluoropropan- |
| | 2-yl)-1H-pyrrole-3-carboxamide |
| 2526. | N-((3-(4-(((3S,4R)-1-ethyl-3-fluoropiperidin-4-yl)amino)-1-(2,2,2- |
| | trifluoroethyl)-1H-indol-2-yl)-1,2,4-oxadiazol-5-yl)methyl)-1-(2-fluoroethyl)- |
| | 1H-pyrrole-3-carboxamide |
| 2527. | N-((3-(4-(((3S,4R)-3-fluoro-1-methylpiperidin-4-yl)amino)-1-(2,2,2- |
| | trifluoroethyl)-1H-indol-2-yl)-1,2,4-oxadiazol-5-yl)methyl)-1-(1- |
| | methoxypropan-2-yl)-1H-pyrrole-3-carboxamide |

### Example 2: Combination of Compound 1 and MDM2 Inhibitors in MTT Cell Assays

One of the key functions of activated p53 within cells is to cause growth arrest and potentially cell death. This complex array of activities can be assessed by following cell numbers over a period of time. One well established method is the MTT assay. The MTT assay is a colorimetric assay for assessing cell metabolic activity. NAD(P)H-dependent cellular oxidoreductase enzymes may, under defined conditions, reflect the number of viable cells present. These enzymes can reduce the tetrazolium dye MTT 3-(4,5-dimethylthiazol-2-yl)-2,5-diphenyltetrazolium bromide to its insoluble formazan, which has a purple color. Tetrazolium dye assays can also be used to measure cytotoxicity (loss of viable cells) or cytostatic activity (shift from proliferation to quiescence) of potential medicinal agents and toxic materials. We used this assay to assess the combination activity of Compound 1 (4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide) with MDM2 inhibitors (Nutlin 3a, RG7112, RG7388) in two human tumor lines were used: NUGC3 (JCRB gastric adenocarcinoma) and T3M4 (RIKEN pancreatic adenocarcinoma) which have homozygous TP53 Y220C mutations. MTT was purchased from Sigma and dissolved in PBS (Sigma) at 5 mg/ml and stored at 4 C. Compounds were dissolved in 100% dimethyl sulfoxide (DMSO) (Sigma) at concentration of 10 mM and stored at −20 C. The combination activity of Compound 1 with MDM2 inhibitors (Nutlin 3a, RG7112, RG7388) was evaluated using the MTT assay in 96-well plate format. Cell viability was determined by measuring the reduction of 3-(4,5-Dimethylthiazol-2-yl)-2,5 diphenyltetrazolium bromide) (MTT) to formazan. Briefly, cells were seeded at a density of 750˜1500 cells per well (depending on the growth rate) in 96-well microtiter plates in a volume of 180 μL growth medium (NUGC3 Growth medium RPMI1640+10% FBS; Seeding density 1500; T3M4 Growth medium DMEM-F12 HAM+10% FBS; Seeding density 750). 180 μL of cell free medium was added to wells for MTT background. Plates were incubated at 37° C. with 5% CO₂ for 24 hours before the addition of Compound 1 and MDM2 inhibitors (Nutlin 3a, RG7112, RG7388). For the human cell line treatment, Compound 1 with MDM2 inhibitors (Nutlin 3a, RG7112, RG7388) was tested at 10 μM followed by 6 additional 2-fold serial dilutions in a combinatorial matrix. Compound 1 and MDM2 inhibitors (Nutlin 3a, RG7112, R^{G}7388) was prepared at 20× the final assay concentration in growth medium containing 2% dimethyl sulfoxide (DMSO) on drug dilution plate, 10 μL of appropriate dilution for each of the two compounds in combination was added to cell culture plate in duplicates. 20 μL of medium contained 2% DMSO was added to the wells for control (CTRL) and MTT background (BK). Antiproliferative activity in combination was assessed 5 days later by the addition of MTT. Cells were incubated with 50 μL per well of MTT at 5 mg/ml for 2 hours at 37 C with 5% CO2. Thereafter, gently aspirated medium, 50 μL of 100% ethanol was added to each well to dissolve the formazan crystals. The conversion of MTT into formazan by viable cells was measured by microplate reader for absorbance with the wavelength of 570 nm and reference wavelength of 650 nm. The results were presented as a percentage of the viability of untreated cells (control), which were regarded as 100% viable using the formula:

$$percent viability = \frac{Mean absorance of experimental wells}{Mean absorance of control wells} \times 100$$

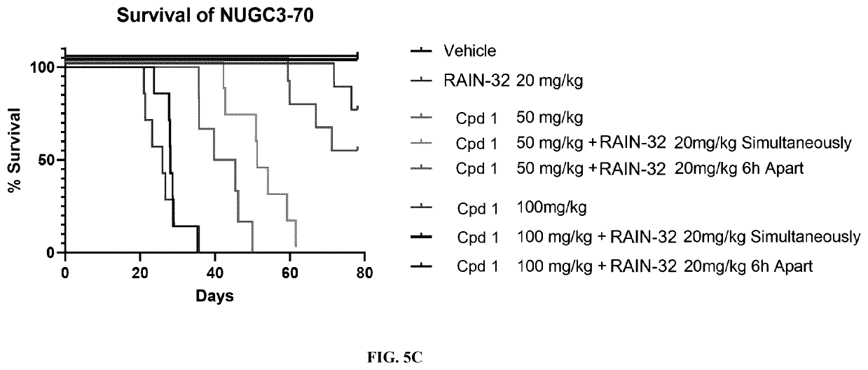

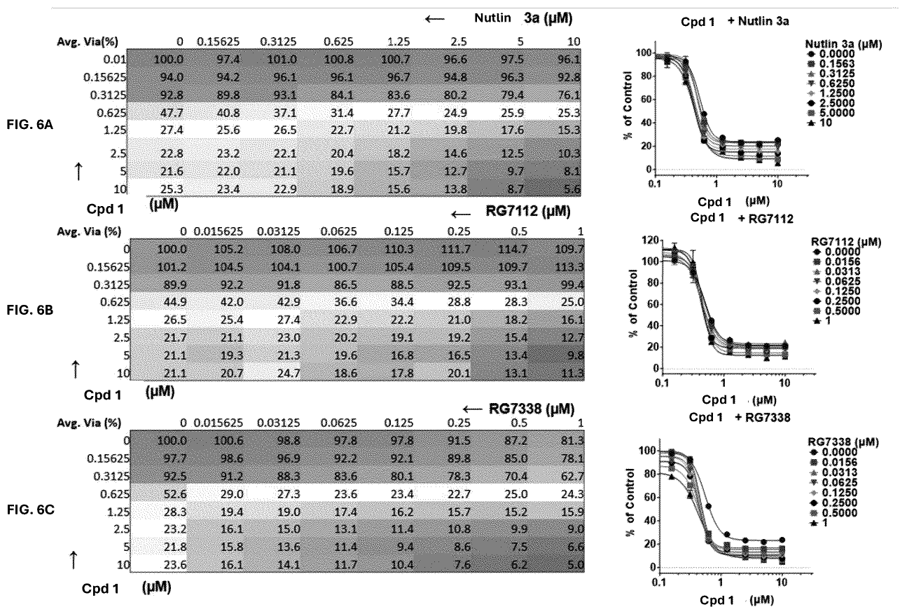

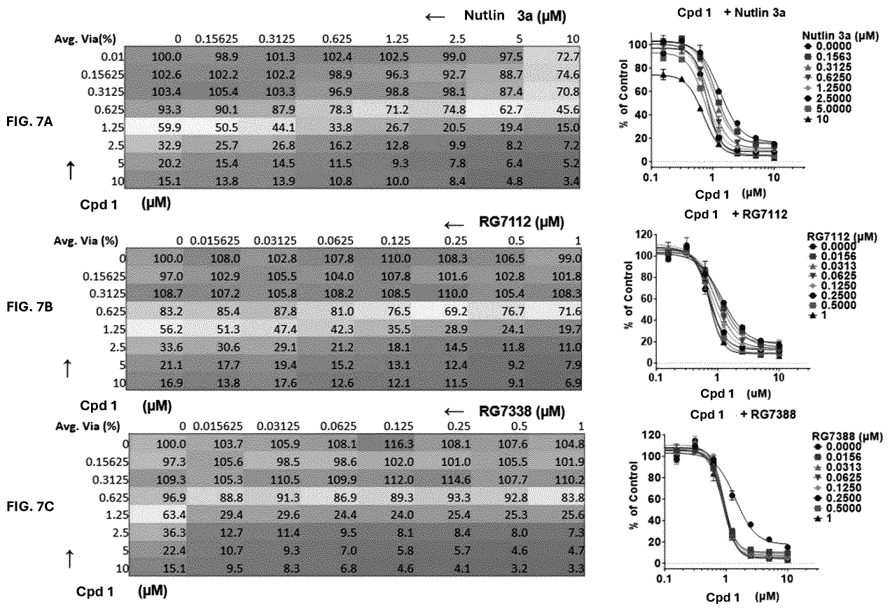

The IC₅₀ was determined from the regression of a plot of the Logarithm of concentration versus percent of viability by XLfit IDBS. Combinations of Compound 1 (4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide) and MDM2 inhibitors shows changes in percent viability (mm³) in an MTT cell model of gastric cancer (NUGC3). When plotted using Compusyn software, the color blue indicates combinations with an additive/synergistic effect (FIGS. 6A-6C). Combinations of Compound 1 and MDM2 inhibitors shows changes in percent viability (mm³) in an MTT cell model of pancreatic cancer (T3M4). When plotted using Compusyn software, the color blue indicates combinations with additive/synergistic effect (FIGS. 7A-7C).

### Example 3: Combination of Compound 1 and MDM2 Inhibitors in Mouse Gastric Cancer Model

Compound 1 (4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide) is an indole compound substituted with a trifluoroethyl group at the 1-position; propynyl amino-methoxy-methylamido phenyl group at the 2-position; and a heterocycle-substituted amino group at the 4-position. When NUGC3 tumors reached ˜200 mm³, mice were administered Vehicle, Compound 1 at 50 mg/kg, or 100 mg/kg QD, Compound 1 at 50 mg/kg simultaneous with MDM2 inhibitors at 20 mg/kg, or 100 mg/kg QD simultaneous with MDM2 inhibitors at 20 mg/kg; Compound 1 at 50 mg/kg six hours separated from dosing with MDM2 inhibitors at 20 mg/kg, or 100 mg/kg QD six hours separated from dosing with MDM2 inhibitors at 20 mg/kg; and MDM2 inhibitors at 20 mg/kg. Tumors were not harvested at the end of the survival study.

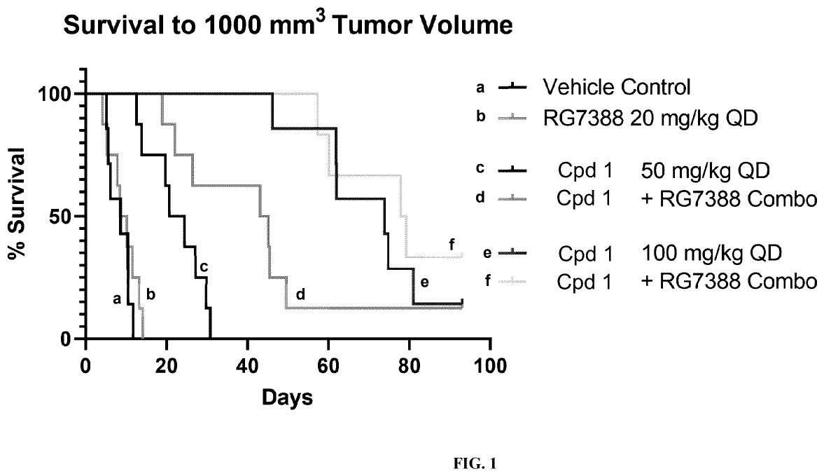

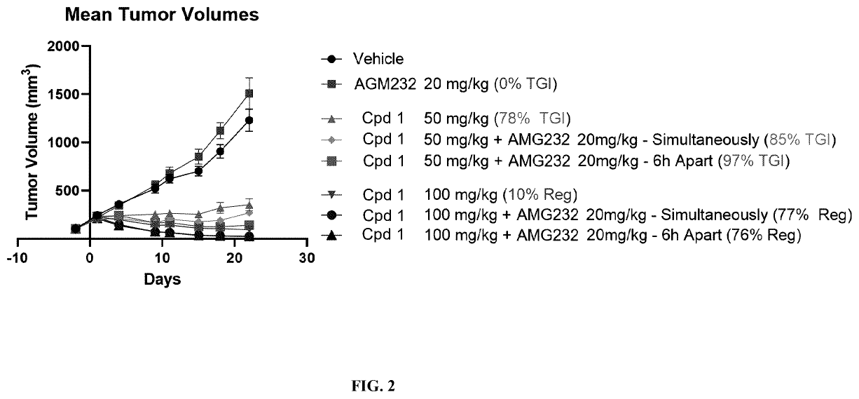

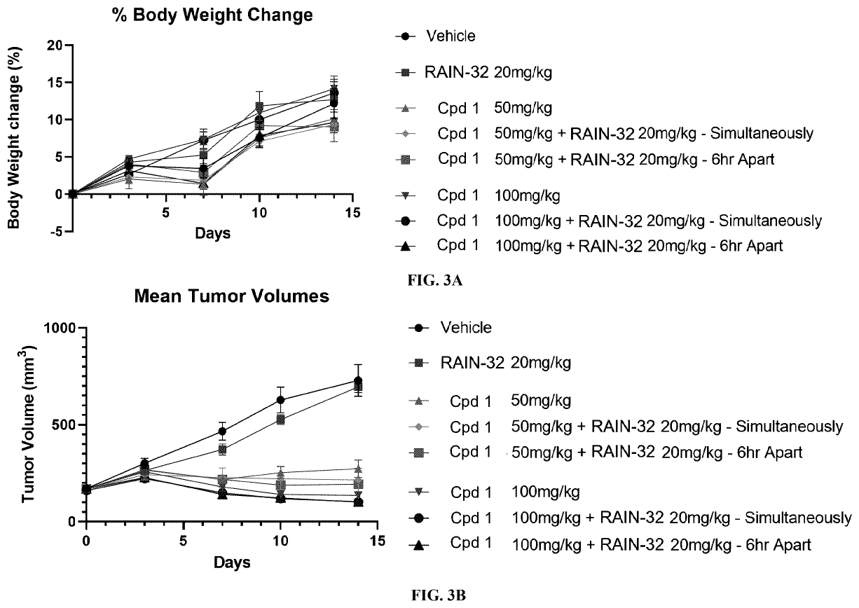

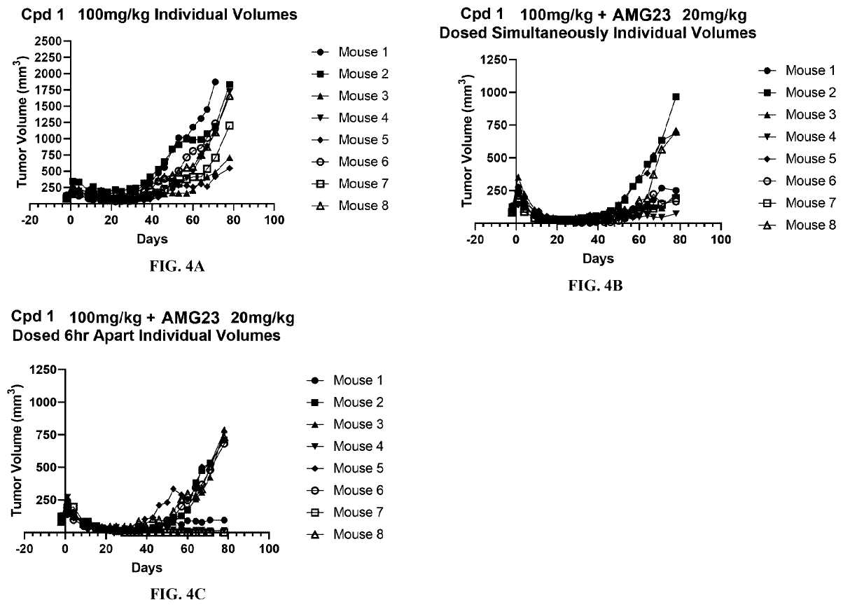

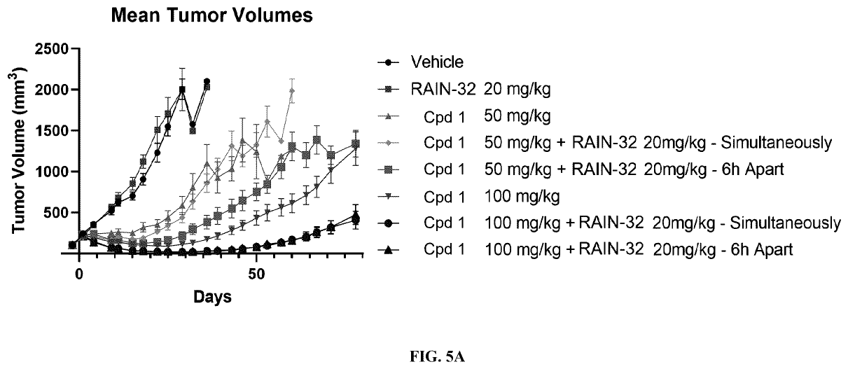

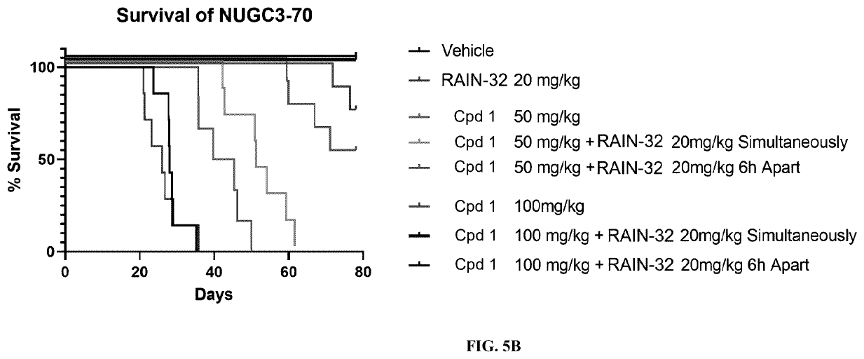

FIG. 1 shows the changes in % survival in a mouse xenograft model of gastric cancer (NUGC3). An increase in Overall Survival was Observed with a combination of Compound 1 and RG7388. When the tumor volumes were measured in a mouse xenograft model of gastric cancer (NUGC3-71), evaluating combinations of Compound 1 and AMG232 (FIG. 2A), reduction of tumor volumes were seen. There was no significant change in body weights (FIG. 2B). Similar results were observed with combinations of Compound 1 and RAIN-32 (FIGS. 3A-3B). Longer evaluation of the combination of Compound 1 (100 mg/kg) and AMG232 identified an improved decrease in tumor volume reduction and an increase in % survival, especially when the two were dosed 6 hour apart (FIGS. 4A-4C and FIGS. 5A-5C).

### Example 4: Combination of Compound 1 and Kinase Inhibitors in CTG Cell Assays

The effect of a combination of Compound 1 and various kinase inhibitors on p53 mediated growth arrest and cell death was assessed by following cell count over a period of time via a CellTiter-Glo® Luminescent Cell Viability Assay (Promega). The CellTiter-Glo® Assay is a homogeneous method to determine the number of viable cells in culture based on quantitation of the ATP present, which signals the presence of metabolically active cells. The CellTiter-Glo® Luminescent Cell Viability Assay was purchased from Promega and used according to manufacturer's instructions. Compounds were dissolved in 100% dimethyl sulfoxide (DMSO) (Sigma) at concentration of 10 mM and stored at −20° C. Briefly, cells [NUGC3 (JCRB gastric adenocarcinoma), T3M4 (RIKEN pancreatic adenocarcinoma), HCC366 (DSMZ, lung adenosquamous carcinoma), HUH-7 (JCRB, hepato cellular carcinoma), MFE-296 (DSMZ, endometrial adenocarcinoma), KON (JCRB, oral squamous carcinoma), SNU—NCC19 (KCLB, intestinal adenocarcinoma), TE-8 (RIKEN, esophagus squamous cell carcinoma), BxPC3 (ATCC, pancreatic adenocarcinoma), COV362 (ECACC, oavarian epithelial-endometroid carcinoma), HCC2935 (ATCC, lung pleural effusion), HCC1419 (ATCC, breast ductal carcinoma)] were seeded at a density of 750˜5000 cells per well (depending on the growth rate) in 96-well microtiter plates in a volume of 120 μL growth medium. (NUGC3 Growth medium: RPMI1640+10% FBS; Seeding density 1500; T3M4 Growth medium: DMEM-F12 HAM+10% FBS; Seeding density 750). 120 μL of cell free medium was added to wells for CTG background. Plates were incubated at 37° C. with 5% CO2 for 24 hours before the addition of Compound 1 and PI3K and AKT inhibitors. For the human cell line treatment, Compound 1 was tested at 10 μM followed by 8 additional 2-fold serial dilutions in a combinatorial matrix. PI3K inhibitors were tested at 10 μM followed by 7 additional 2-fold serial dilutions in a combinatorial matrix. AKT inhibitors were tested at 10 μM followed by 5 additional 3-fold serial dilutions in a combinatorial matrix. Antiproliferative activity in combination was assessed 5 days later by the addition of CTG. 50 μL CTG was added per well, cells incubated for 10 mins with gentle shaking at room temperature, and luminescence was then read at 1000 ms. The results were presented as a percentage of the viability of untreated cells (control), which were regarded as 100% viable.

Results were also analyzed by Combenefit software (Roell K R, Reif D M, Motsinger-Reif A A. An introduction to terminology and methodology of chemical synergy-perspectives from across disciplines. Front Pharmacol. 2017; 8:158), which enables the visualization, analysis and quantification of drug combination effects such as synergy and antagonism using classical Synergy models (Loewe, Bliss, HSA). If the actual effect of a drug combination was greater than the additive effect, the synergy score was larger than zero. If the actual effect of a drug combination was less than the additive effect the synergy score was less than zero. Thus, higher synergy scores imply synergy and lower (negative) scores imply antagonism.

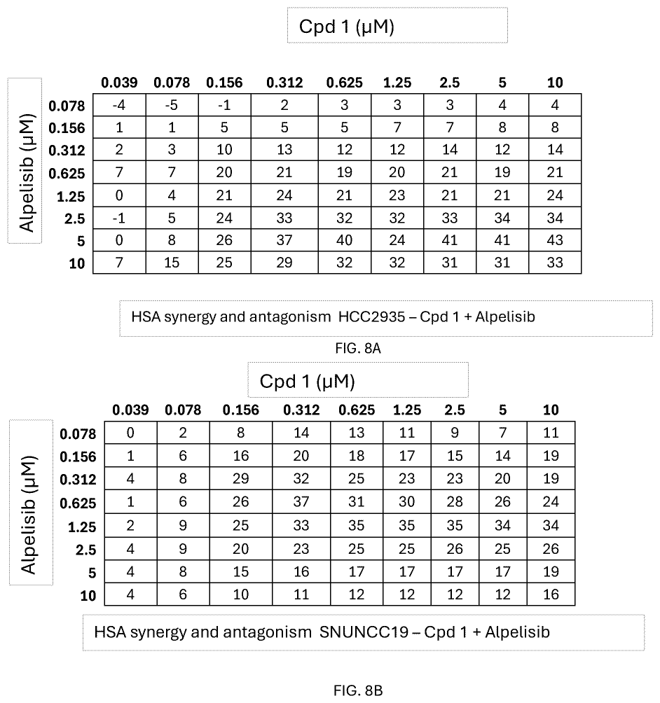

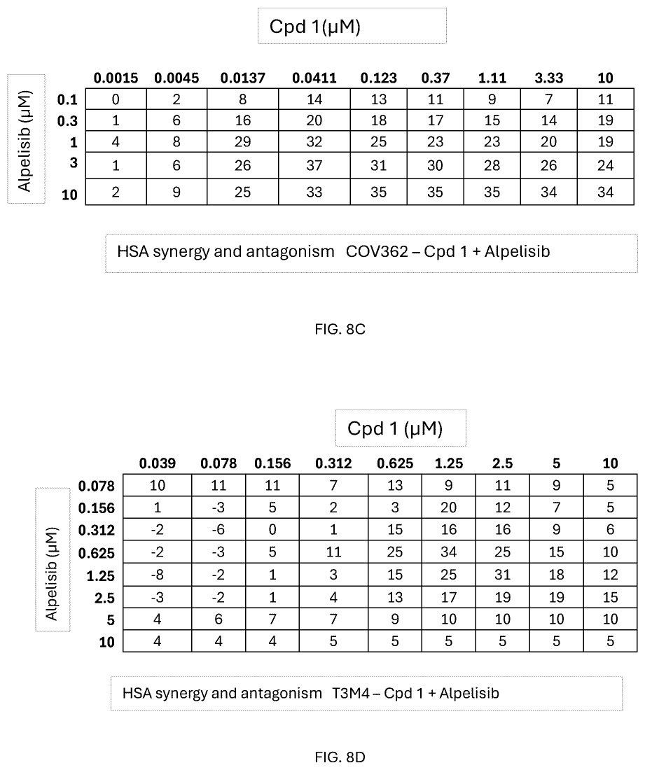

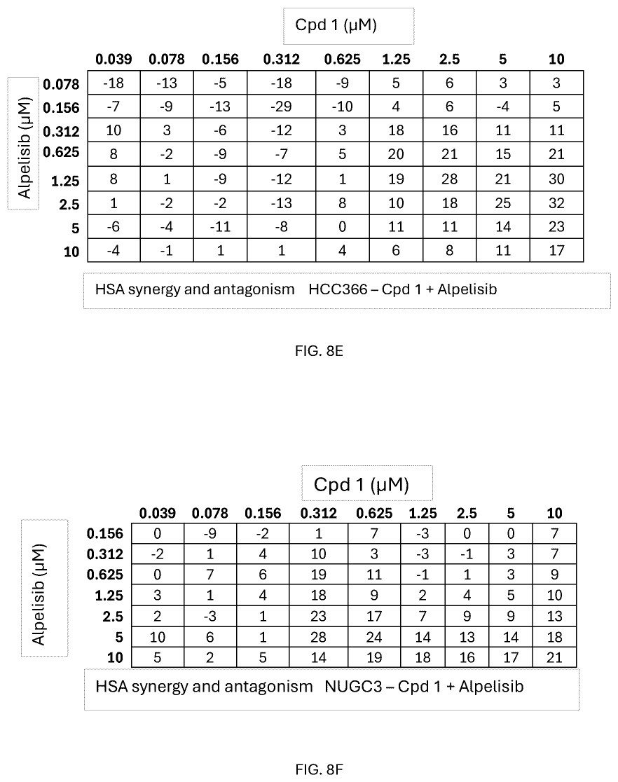

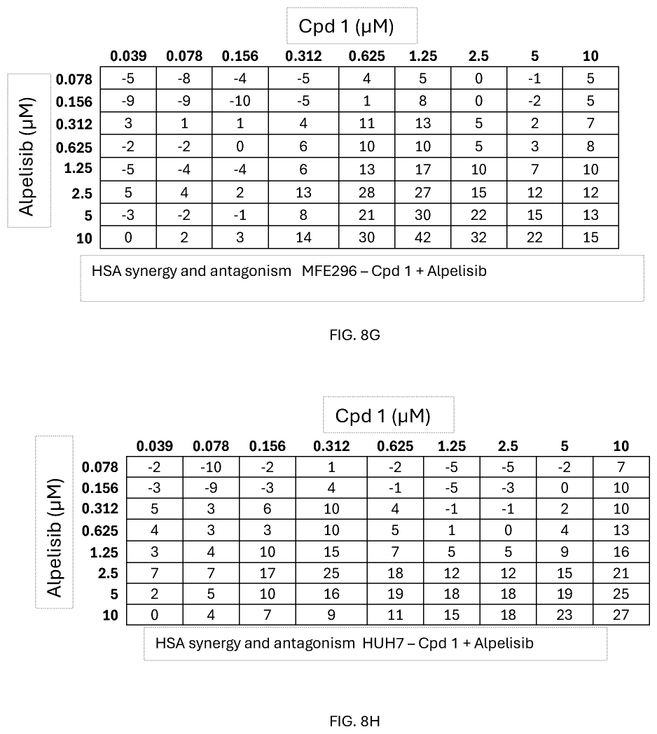

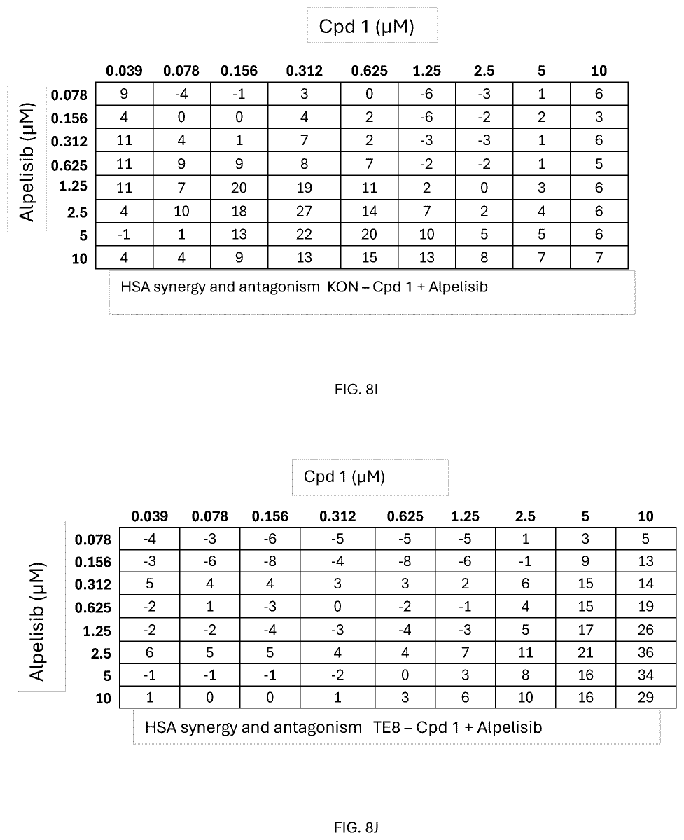

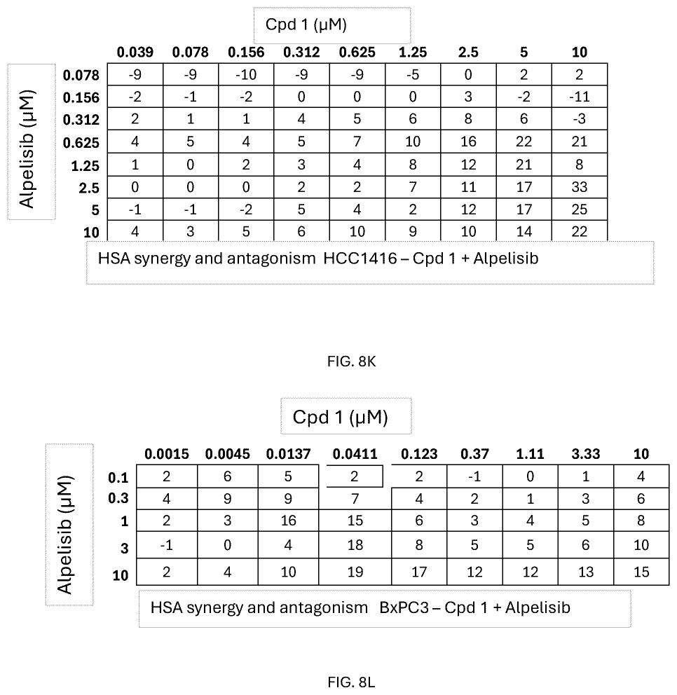

FIGS. 8A-8L show highest single agent (HSA) synergy scores between PI3K inhibitor alpelisib (0.078-10 mM) and Compound 1 (0.039-10 mM) as calculated by Combenefit software in HCC2935 cells (FIG. 8A), SNUNCC19 cells (FIG. 8B), COV362 cells (FIG. 8C), T3M4 cells (FIG. 8D), HCC366 cells (FIG. 8E), NUGC3 cells (FIG. 8F), MFE296 cells (FIG. 8G), HUH7 cells (FIG. 8H), KON cells (FIG. 8I), TE8 cells (FIG. 8J), HCC1416 cells (FIG. 8K), and BxPC3 cells (FIG. 8L).

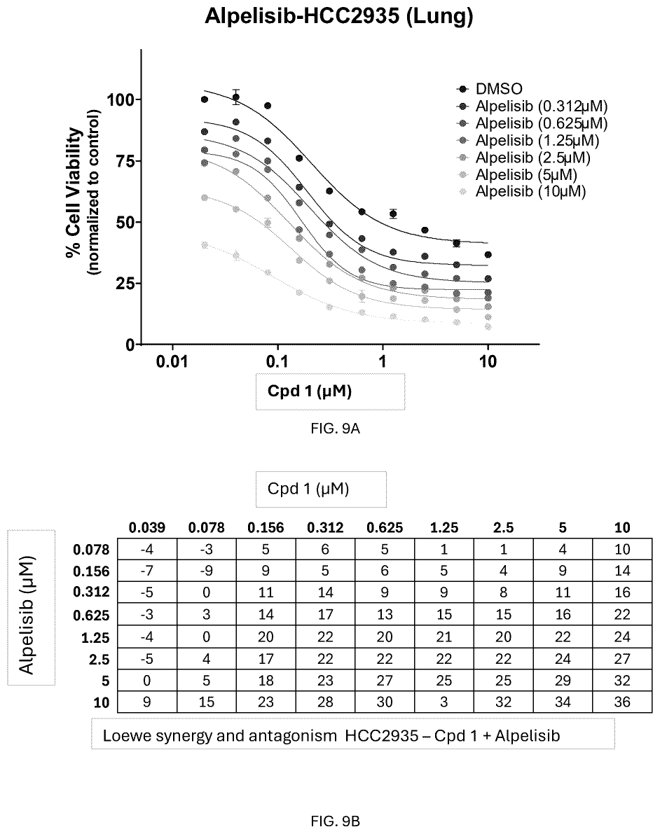

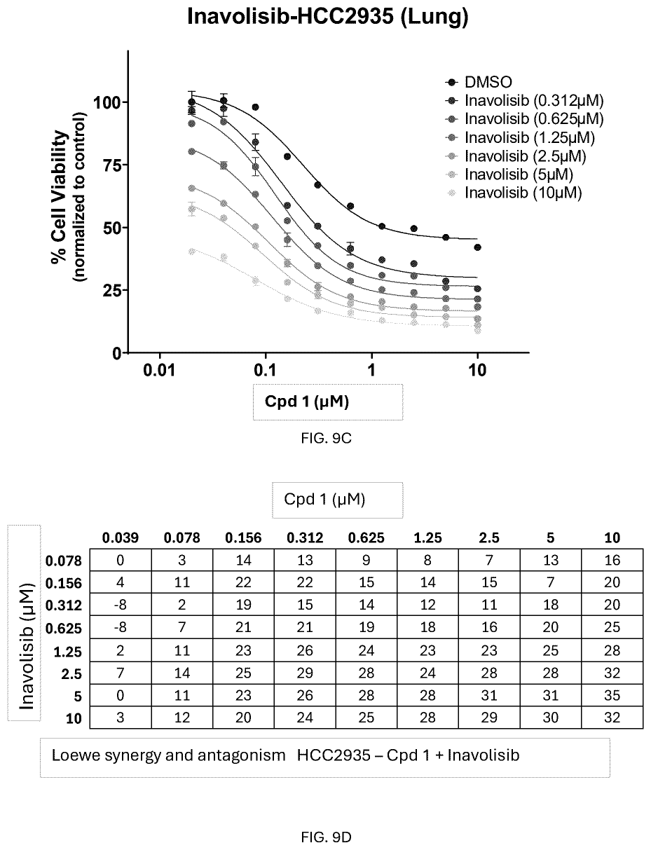

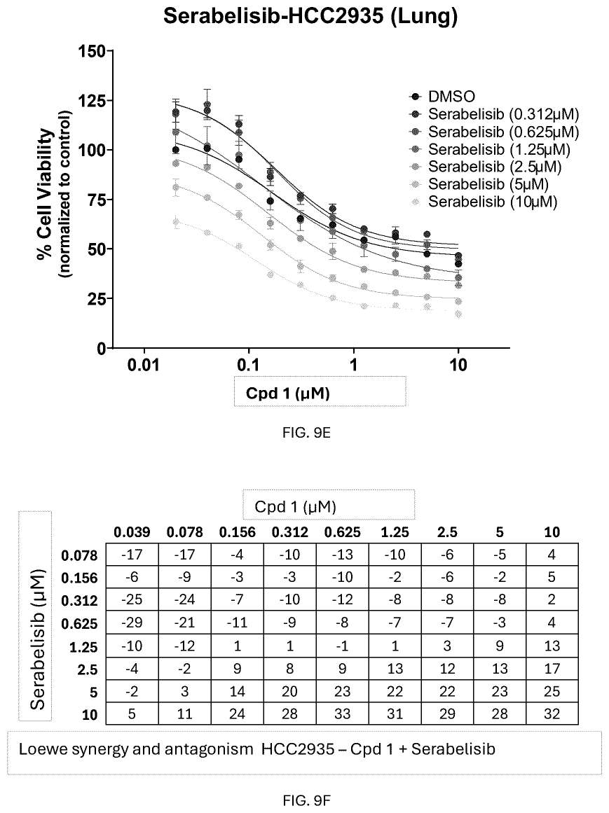

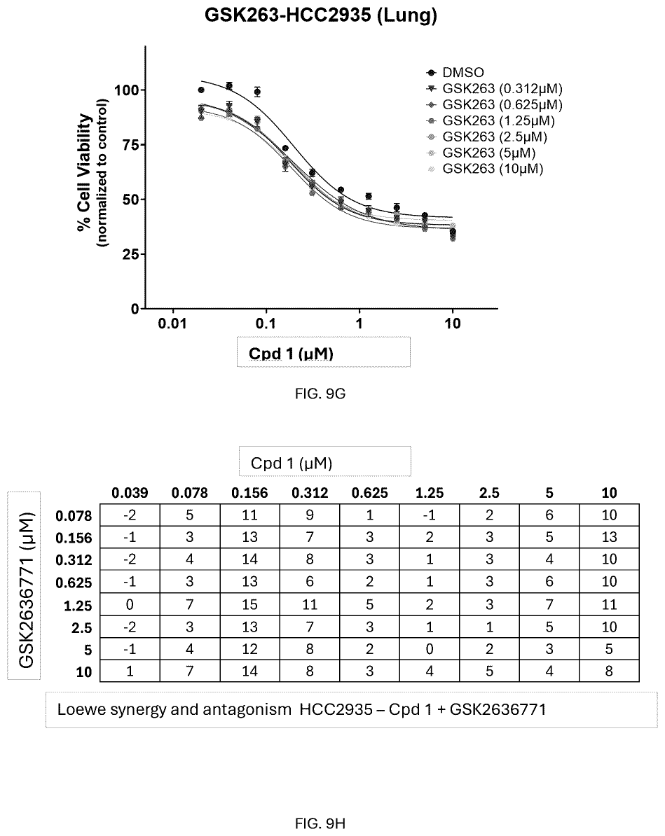

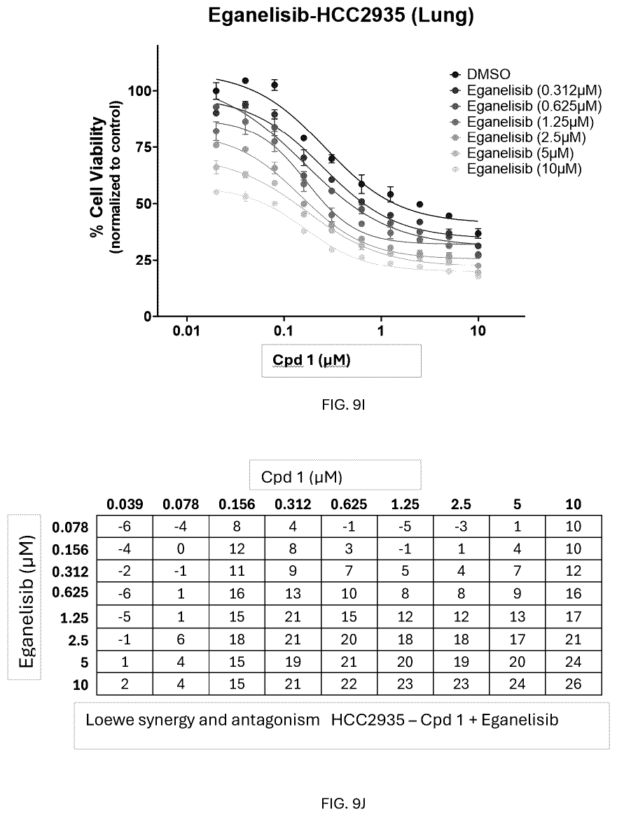

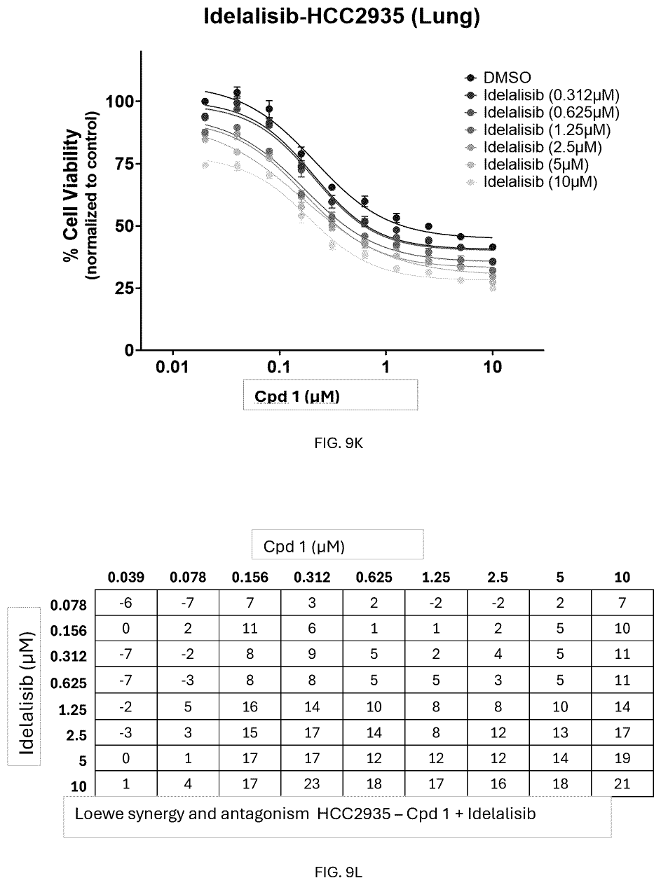

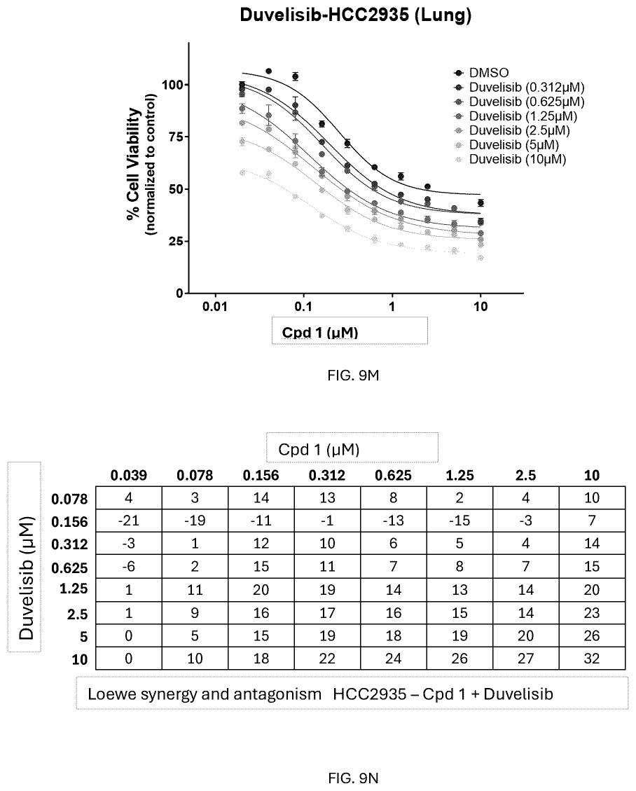

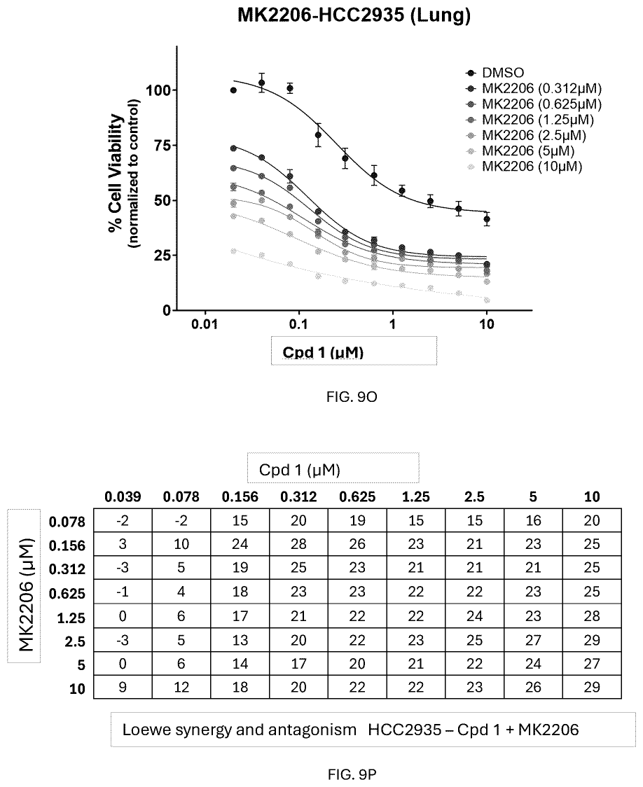

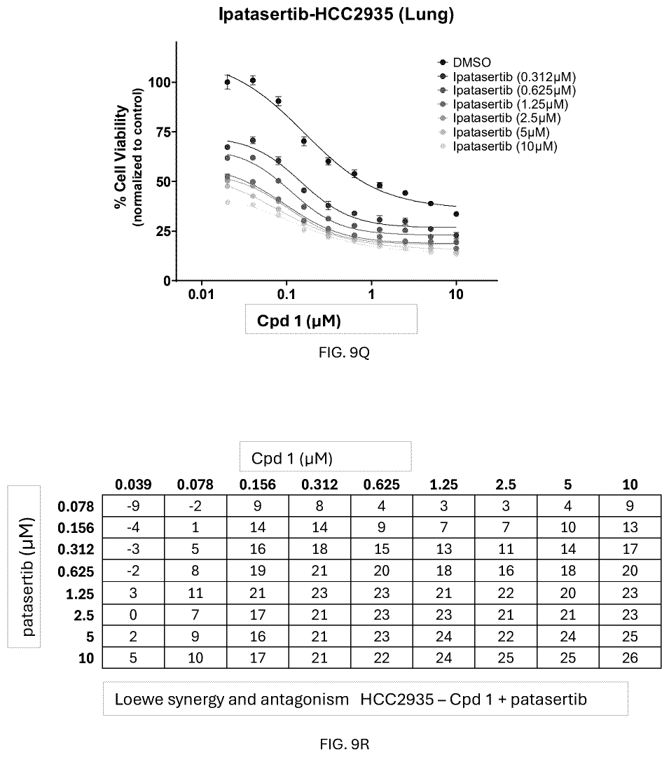

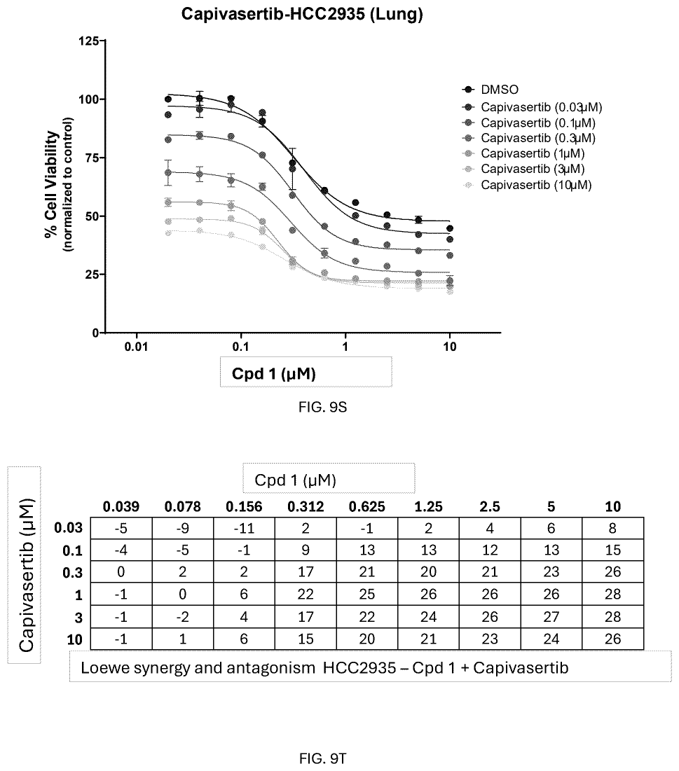

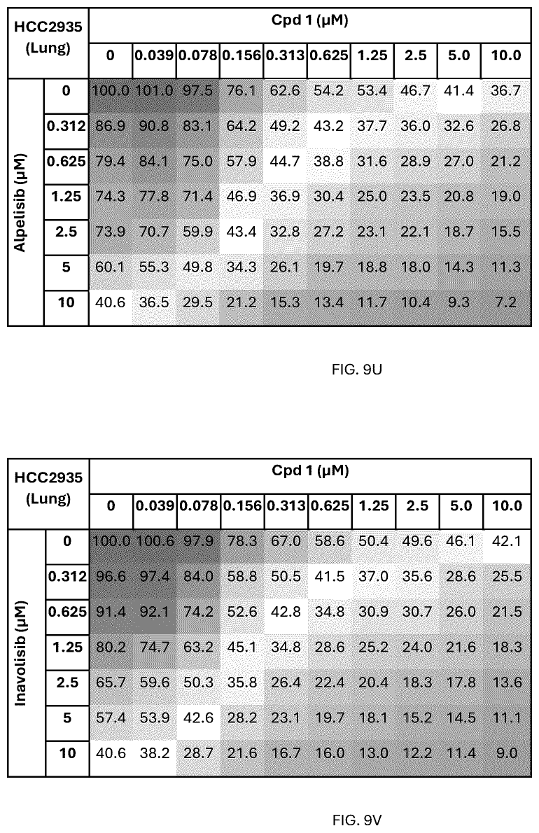

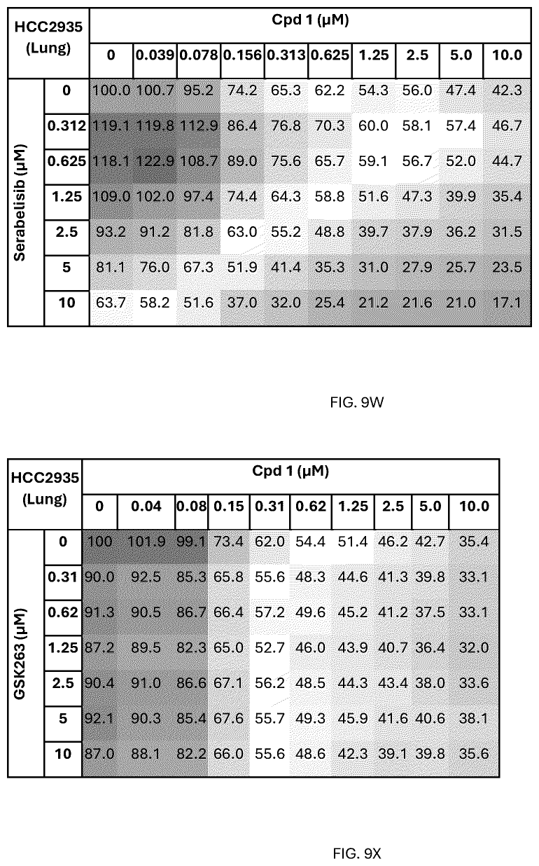

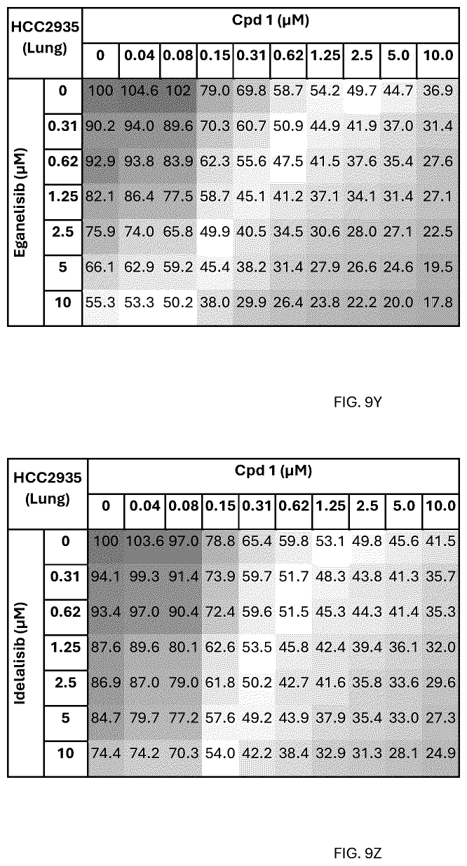

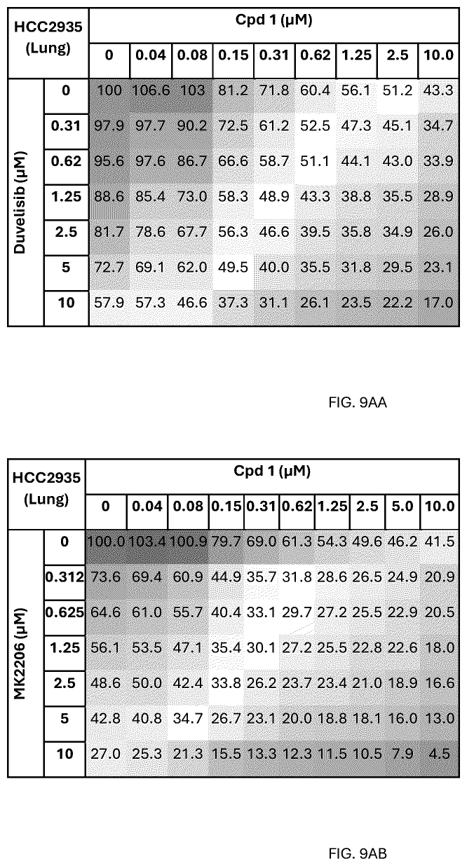

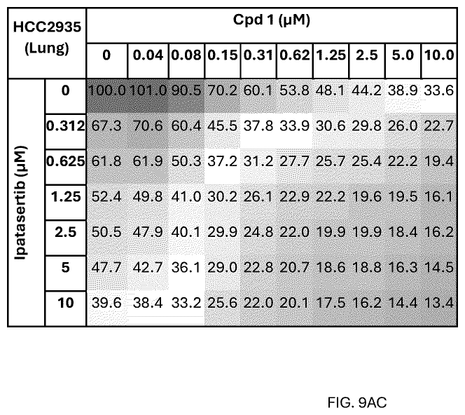

FIGS. 9A-9B show changes in percent viability (FIG. 9A) and Loewe synergy scores as calculated by Combenefit software (FIG. 9B) in a CTG cell model of lung cancer (HCC2935) treated with vehicle, or PI3K inhibitor alpelisib (0.078-10 mM) together with Compound 1 (0.039-10 mM).

FIGS. 9C-9D show changes in percent viability (FIG. 9C) and Loewe synergy scores as calculated by Combenefit software (FIG. 9D) in a CTG cell model of lung cancer (HCC2935) treated with vehicle, or PI3K inhibitor inavolisib (0.078-10 mM) together with Compound 1 (0.039-10 mM).

FIGS. 9E-9F show changes in percent viability (FIG. 9E) and Loewe synergy scores as calculated by Combenefit software (FIG. 9F) in a CTG cell model of lung cancer (HCC2935) treated with vehicle, or PI3K inhibitor serabelisib (0.07810 mM) together with Compound 1 (0.039-10 mM).

FIGS. 9G-9H show changes in percent viability (FIG. 9G) and Loewe synergy scores as calculated by Combenefit software (FIG. 9H) in a CTG cell model of lung cancer (HCC2935) treated with vehicle, or PI3K inhibitor GSK2636771 (GSK263) (0.078-10 mM) together with Compound 1 (0.039-10 mM).

FIGS. 9I-9J show changes in percent viability (FIG. 9I) and Loewe synergy scores as calculated by Combenefit software (FIG. 9J) in a CTG cell model of lung cancer (HCC2935) treated with vehicle, or PI3K inhibitor eganelisib (0.078-10 mM) together with Compound 1 (0.039-10 mM).

FIGS. 9K-9L show changes in percent viability (FIG. 9K) and Loewe synergy scores as calculated by Combenefit software (FIG. 9L) in a CTG cell model of lung cancer (HCC2935) treated with vehicle, or PI3K inhibitor idelalisib (0.078-10 mM) together with Compound 1 (0.039-10 mM).

FIGS. 9M-9N show changes in percent viability (FIG. 9M) and Loewe synergy scores as calculated by Combenefit software (FIG. 9N) in a CTG cell model of lung cancer (HCC2935) treated with vehicle, or PI3K inhibitor duvelisib (0.078-10 mM) together with Compound 1 (0.039-10 mM).

FIGS. 9O-9P show changes in percent viability (FIG. 9O) and Loewe synergy scores as calculated by Combenefit software (FIG. 9P) in a CTG cell model of lung cancer (HCC2935) treated with vehicle, or AKT inhibitor MK2206 (0.078-10 mM) together with Compound 1 (0.039-10 mM).

FIGS. 9Q-9R show changes in percent viability (FIG. 9Q) and Loewe synergy scores as calculated by Combenefit software (FIG. 9R) in a CTG cell model of lung cancer (HCC2935) treated with vehicle, or AKT inhibitor ipataserib (0.078-10 mM) together with Compound 1 (0.039-10 mM).

FIGS. 9S-9T show changes in percent viability (FIG. 9S) and Loewe synergy scores as calculated by Combenefit software (FIG. 9T) in a CTG cell model of lung cancer (HCC2935) treated with vehicle, or AKT inhibitor capivasertib (0.03-10 mM) together with Compound 1 (0.039-10 mM).

FIGS. 9U-AC show percent viability in HCC2935 cells treated with Compound 1 (0.04-10 mM) in combination with alpelisib (FIG. 9U), inavolisib (FIG. 9V), serabelisib (FIG. 9W), GSK2636771 (GSK263) (FIG. 9X), eganelisib (FIG. 9Y), idelalisib (FIG. 9Z), duvelisib (FIG. 9AA), MK2206 (FIG. 9AB), and ipatasertib (FIG. 9AC).

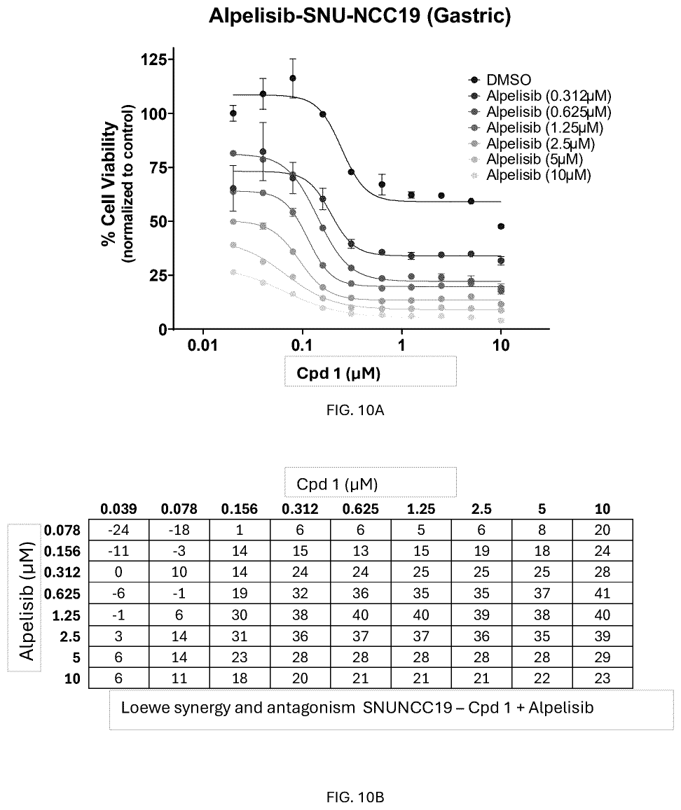

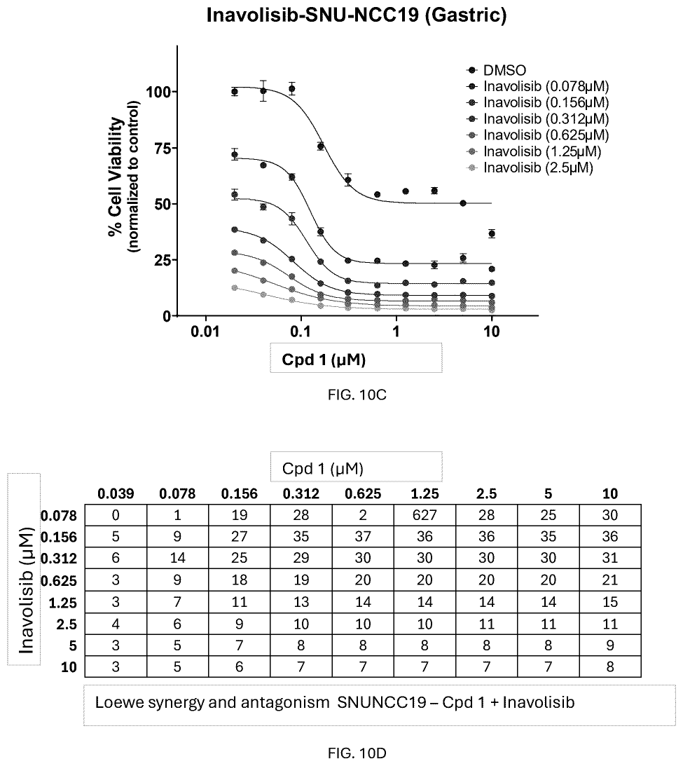

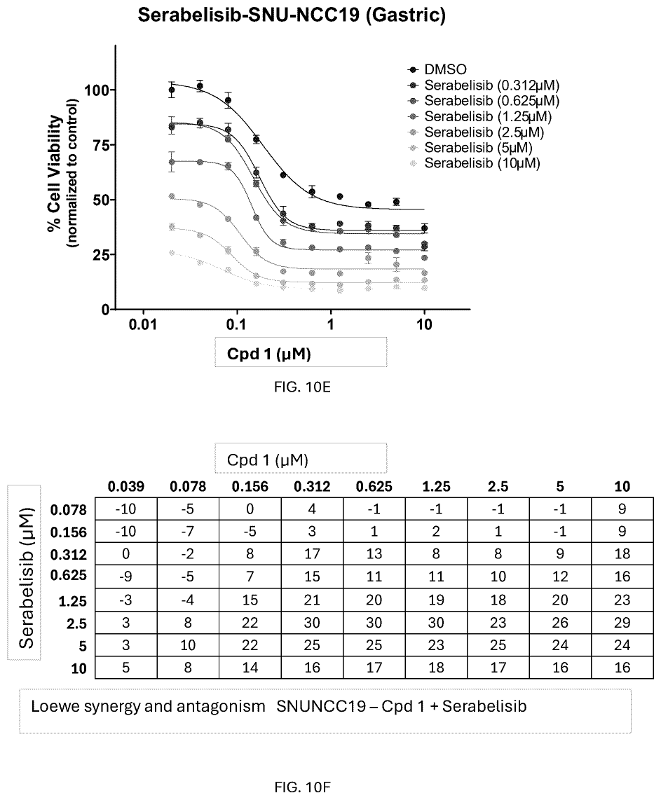

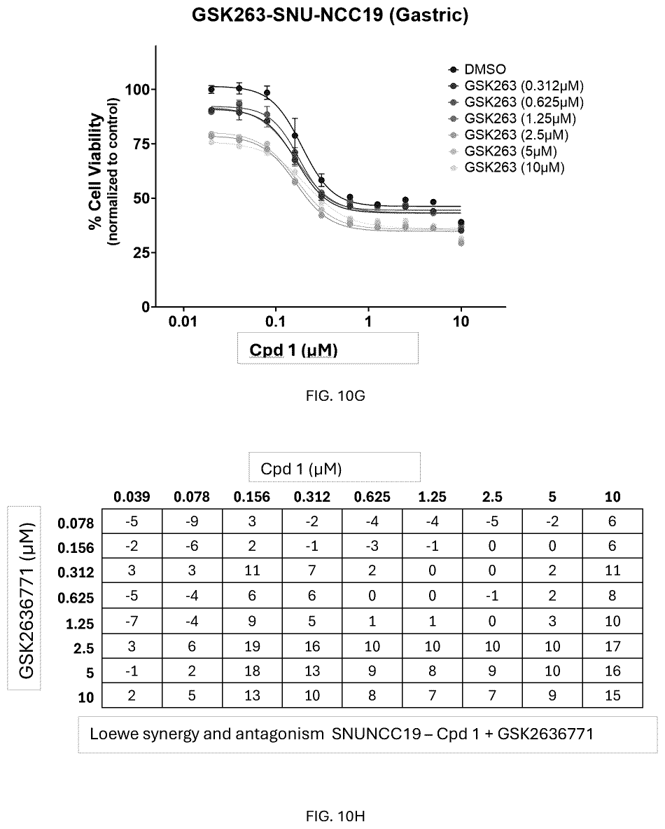

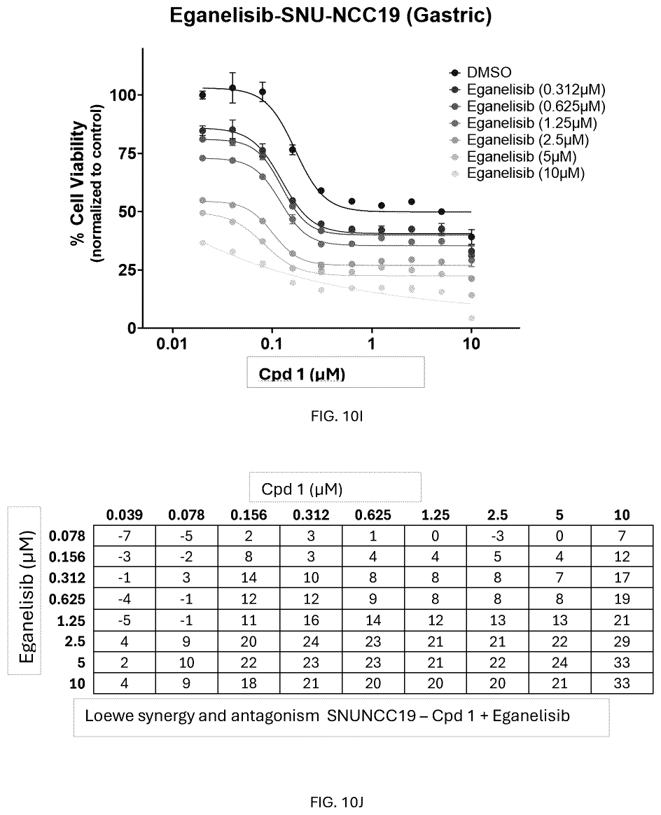

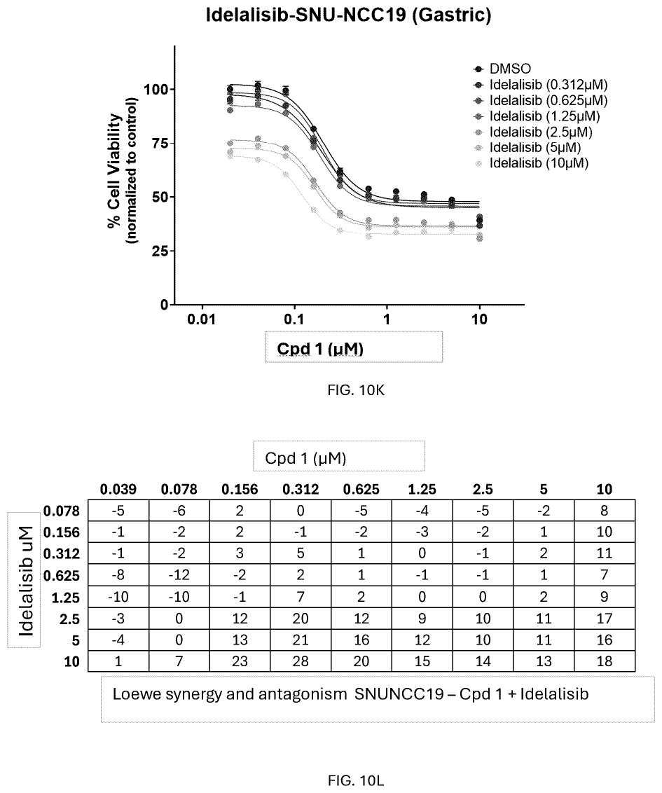

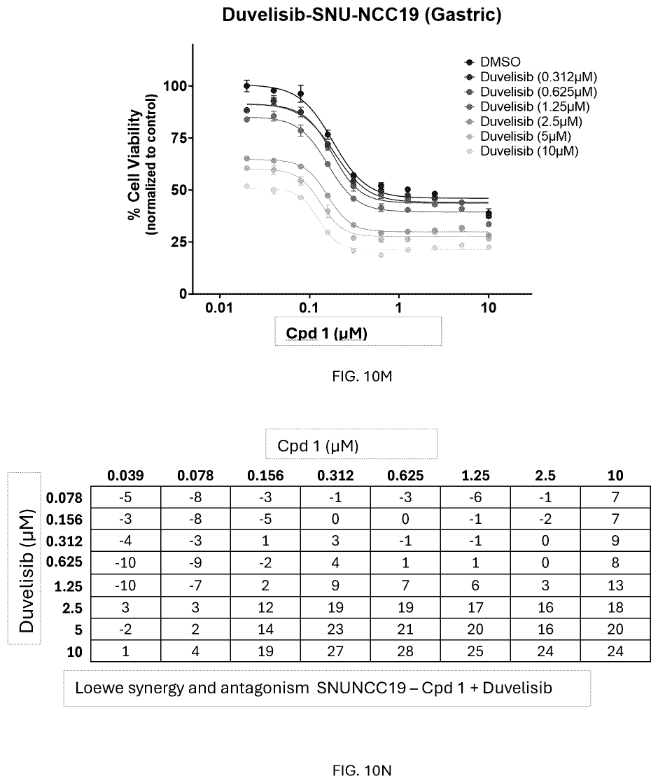

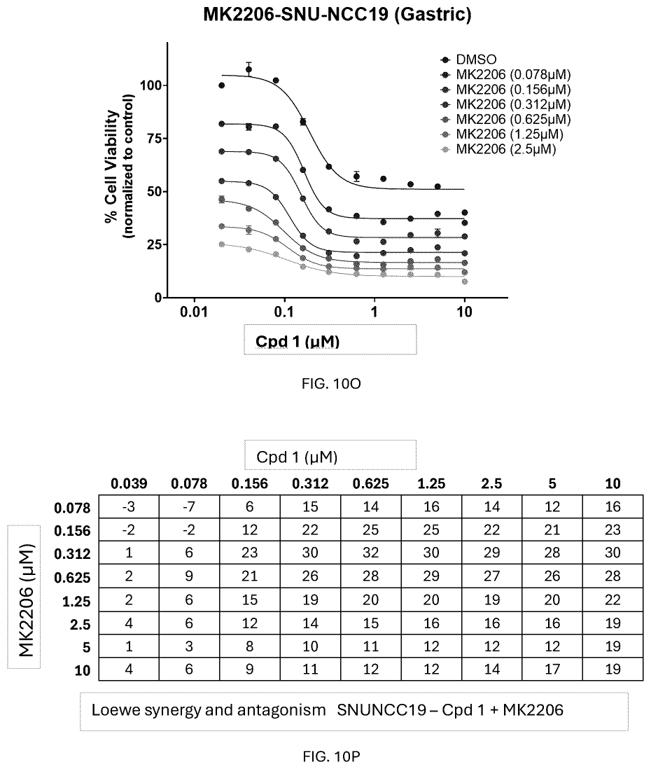

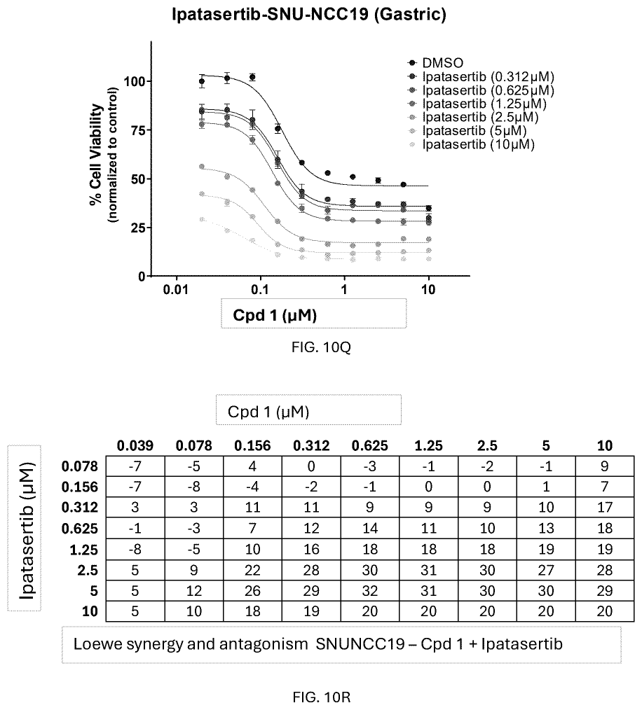

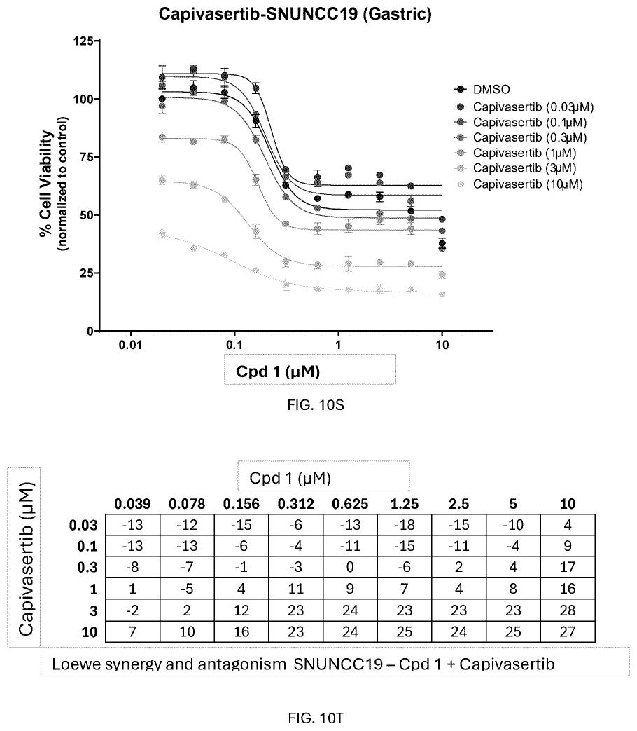

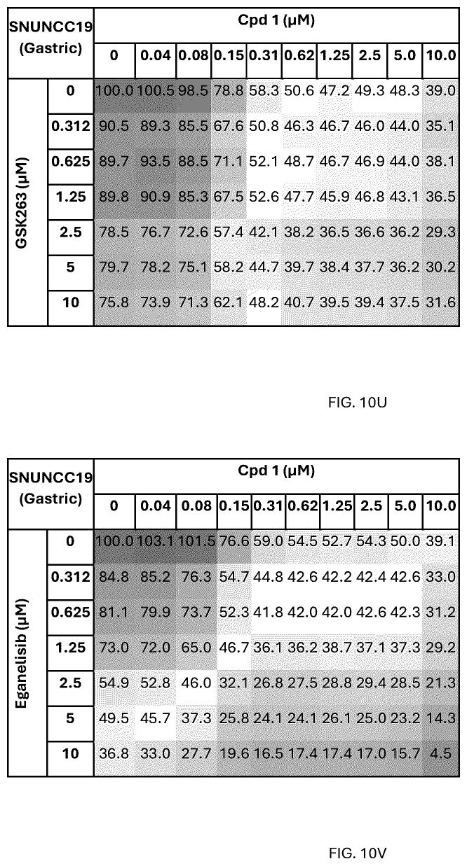

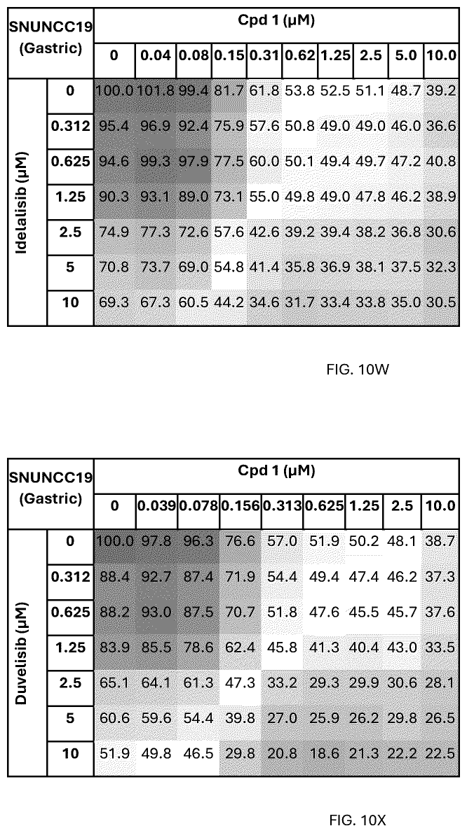

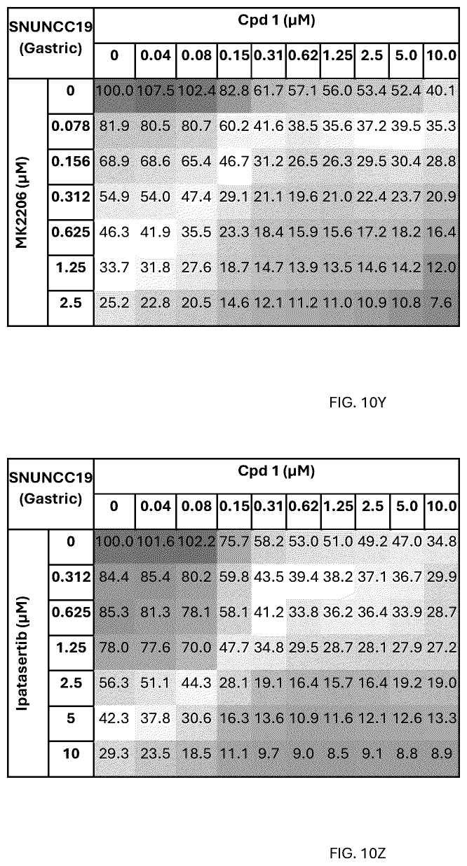

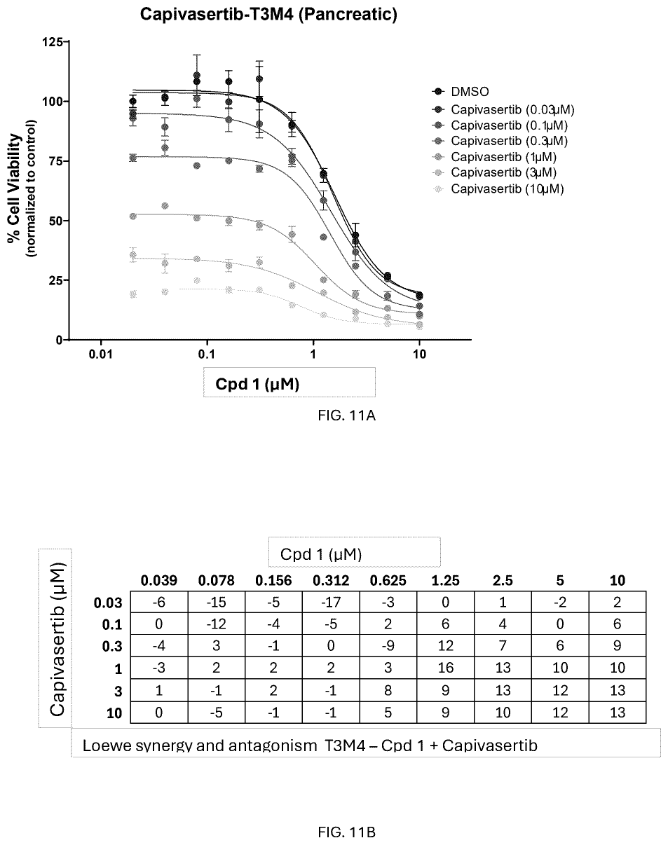

FIGS. 10A-10B show changes in percent viability (FIG. 10A) and Loewe synergy scores as calculated by Combenefit software (FIG. 10B) in a CTG cell model of gastric cancer (SNUNCC19) treated with vehicle, or PI3K inhibitor alpelisib (0.078-10 mM) together with Compound 1 (0.039-10 mM).

FIGS. 10C-10D show changes in percent viability (FIG. 10C) and Loewe synergy scores as calculated by Combenefit software (FIG. 10D) in a CTG cell model of gastric cancer (SNUNCC19) treated with vehicle, or PI3K inhibitor inavolisib (0.078-10 mM) together with Compound 1 (0.039-10 mM).

FIGS. 10E-10F show changes in percent viability (FIG. 10E) and Loewe synergy scores as calculated by Combenefit software (FIG. 10F) in a CTG cell model of gastric cancer (SNUNCC19) treated with vehicle, or PI3K inhibitor serabelisib (0.078-10 mM) together with Compound 1 (0.039-10 mM).

FIGS. 10G-10H show changes in percent viability (FIG. 10G) and Loewe synergy scores as calculated by Combenefit software (FIG. 10H) in a CTG cell model of gastric cancer (SNUNCC19) treated with vehicle, or PI3K inhibitor GSK2636771 (GSK263) (0.078-10 mM) together with Compound 1 (0.039-10 mM).

FIGS. 10I-10J show changes in percent viability (FIG. 10I) and Loewe synergy scores as calculated by Combenefit software (FIG. 10J) in a CTG cell model of gastric cancer (SNUNCC19) treated with vehicle, or PI3K inhibitor eganelisib (0.078-10 mM) together with Compound 1 (0.039-10 mM).

FIGS. 10K-10L show changes in percent viability (FIG. 10K) and Loewe synergy scores as calculated by Combenefit software (FIG. 10L) in a CTG cell model of gastric cancer (SNUNCC19) treated with vehicle, or PI3K inhibitor idelalisib (0.078-10 mM) together with Compound 1 (0.039-10 mM).

FIGS. 10M-10N show changes in percent viability (FIG. 10M) and Loewe synergy scores as calculated by Combenefit software (FIG. 10N) in a CTG cell model of gastric cancer (SNUNCC19) treated with vehicle, or PI3K inhibitor duvelisib (0.078-10 mM) together with Compound 1 (0.039-10 mM).

FIGS. 10O-10P show changes in percent viability (FIG. 10O) and Loewe synergy scores as calculated by Combenefit software (FIG. 10P) in a CTG cell model of gastric cancer (SNUNCC19) treated with vehicle, or AKT inhibitor MK2206 (0.078-10 mM) together with Compound 1 (0.039-10 mM).

FIGS. 10Q-10R show changes in percent viability (FIG. 10Q) and Loewe synergy scores as calculated by Combenefit software (FIG. 10R) in a CTG cell model of gastric cancer (SNUNCC19) treated with vehicle, or AKT inhibitor ipataserib (0.078-10 mM) together with Compound 1 (0.039-10 mM). FIGS. 10S-10T show changes in percent viability (FIG. 10S) and Loewe synergy scores as calculated by Combenefit software (FIG. 10T) in a CTG cell model of gastric cancer (SNUNCC19) treated with vehicle, or AKT inhibitor capivasertib (0.078-10 mM) together with Compound 1 (0.039-10 mM).

FIGS. 10U-Z show percent viability in SNUNCC19 cells treated with Compound 1 (0.04-10 mM) in combination with GSK2636771 (GSK263) (FIG. 10U), eganelisib (FIG. 10V), idelalisib (FIG. 10W), duvelisib (FIG. 10X), MK2206 (FIG. 10Y), and ipatasertib (FIG. 10Z).

FIGS. 11A-11B show changes in percent viability (FIG. 11A) and Loewe synergy scores as calculated by Combenefit software (FIG. 11B) in a CTG cell model of pancreatic cancer (T3M4) treated with vehicle, or AKT inhibitor capivasertib (0.03-10 mM) together with Compound 1 (0.039-10 mM).

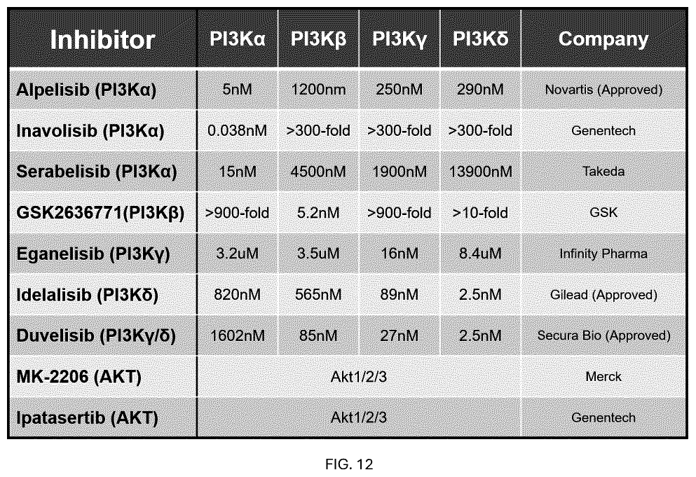

FIG. 12 provides a list of PI3K and AKT inhibitors.

### Example 5: Combination of Compound 1 and Alpesilib in Mouse Gastric Cancer Model and Pancreatic Cancer Model

The effect of a combination of Compound 1 (4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide and alpesilib was assessed in a mouse NUGC-3 gastric cancer model and a T3M-4 pancreatic cancer model.

NUGC-3 model. When NUGC-3 tumors reached ˜200 mm³, mice were administered vehicle, alpelisib at 20 mg/kg, Compound 1 at 50 mg/kg, or 100 mg/kg QD Compound 1 at 50 mg/kg simultaneously with alpelisib at 20 mg/kg, or 100 mg/kg QD Compound 1 simultaneously with alpelisib at 20 mg/kg. Tumors were harvested 4 and 24 h post dose for analysis of downstream biomarkers.

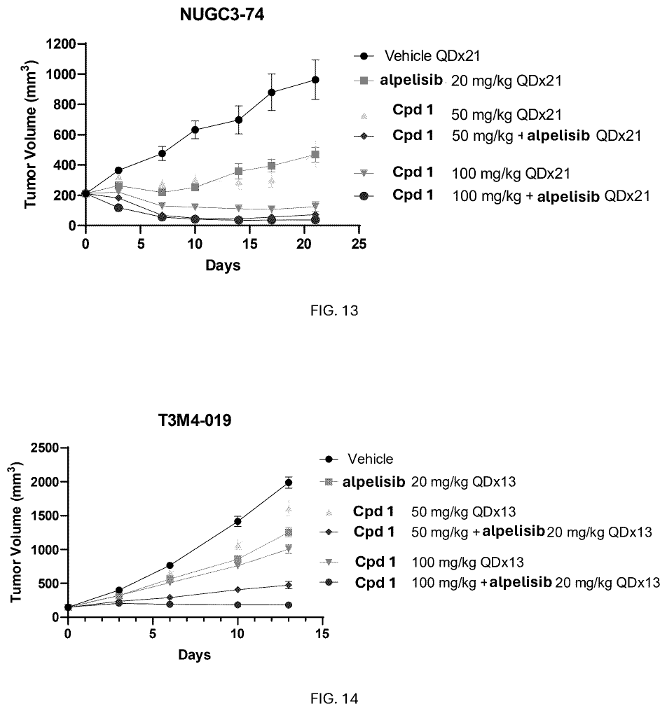

FIG. 13 shows the changes in tumor volume (mm³) in the mouse xenograft NUGC-3 model of gastric cancer. Tumors treated with combinations exhibited decreased volumes compared to tumors treated with single agents. There was no significant decrease in percent body weight change from baseline.

T3M-4 model. When T3M-4 tumors reached an average of −150 mm³, mice were administered vehicle, alpelisib at 20 mg/kg, Compound 1 at 50 mg/kg, or 100 mg/kg QD Compound 1 at 50 mg/kg simultaneously with alpelisib at 20 mg/kg, or 100 mg/kg QD Compound 1 simultaneously with alpelisib at 20 mg/kg. Tumors were harvested 4 and 24 h post dose for analysis of downstream biomarkers.

FIG. 14 shows the changes in tumor volume (mm³) in the mouse xenograft T3M-4 model of pancreatic cancer. Tumors treated with combinations exhibited decreased volumes compared to tumors treated with single agents. There was no significant decrease in percent body weight change from baseline.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs.

1-63. (canceled)

64. A combination comprising:
(a) Compound 1, wherein Compound 1 is: 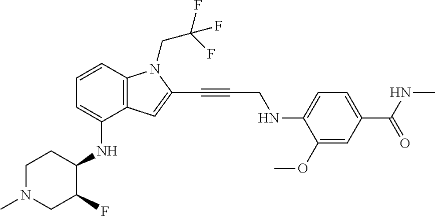
or a pharmaceutically-acceptable salt thereof, and
(b) an AKT inhibitor selected from A6730, B2311, 124018, afuresertib, perifosine, ipatasertib, RX-0201, VQD-002, LY294002, A-443654, A-674563, Akti-1, Akti-2, Akti-1/2, AR-42, API-59CJ-OMe, ATI-13148, AZD-5363, erucylphosphocholine, GSK-2141795, MK2206, KP372-1, L-418, NL-71-101, PBI-05204, PIA5, PX-316, SR13668, triciribine, GSK 690693, FPA 124, Miltefosine, capivasertib, PHT-427, 10-DEBC hydrochloride, Akt inhibitor III, Akt inhibitor VIII, MK-2206 dihydrochloride, SC79, AT7867, CCT128930, A-674563, AGL 2263, 5-benzo[1,3]dioxol-5-ylmethylene-thiazolidine-2,4-dione, BML-257, XL-418, CAS #612847-09-3, CAS #98510-80-6, CAS #127243-85-0, OXY-1 1 1 A, 3-[1-[[4-(7-phenyl-3H-imidazo[4,5-g]quinoxalin-6-yl)phenyl]methyl]piperidin-4-yl]-1H-benzimidazol-2-one, and a pharmaceutically-acceptable salt of any one of the foregoing.

65. The combination of claim 64, wherein Compound 1 and the AKT inhibitor are in the same composition.

66. The combination of claim 64, wherein Compound 1 and the AKT inhibitor are in separate compositions.

67. The combination of claim 64, wherein the AKT inhibitor is MK2206, or a pharmaceutically-acceptable salt thereof.

68. The combination of claim 64, wherein the AKT inhibitor is ipatasertib, or a pharmaceutically-acceptable salt thereof.

69. The combination of claim 64, wherein the AKT inhibitor is capivasertib, or a pharmaceutically-acceptable salt thereof.

70. A method of treating cancer in a subject, the method comprising:
(a) administering to the subject Compound 1, wherein Compound 1 is: 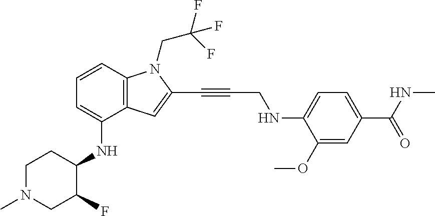
or a pharmaceutically-acceptable salt thereof, and
(b) administering to the subject an AKT inhibitor selected from A6730, B2311, 124018, afuresertib, perifosine, ipatasertib, RX-0201, VQD-002, LY294002, A-443654, A-674563, Akti-1, Akti-2, Akti-1/2, AR-42, API-59CJ-OMe, ATI-13148, AZD-5363, erucylphosphocholine, GSK-2141795, MK2206, KP372-1, L-418, NL-71-101, PBI-05204, PIA5, PX-316, SR13668, triciribine, GSK 690693, FPA 124, Miltefosine, capivasertib, PHT-427, 10-DEBC hydrochloride, Akt inhibitor III, Akt inhibitor VIII, MK-2206 dihydrochloride, SC79, AT7867, CCT128930, A-674563, AGL 2263, 5-benzo[1,3]dioxol-5-ylmethylene-thiazolidine-2,4-dione, BML-257, XL-418, CAS #612847-09-3, CAS #98510-80-6, CAS #127243-85-0, OXY-1 1 1 A, 3-[1-[[4-(7-phenyl-3H-imidazo[4,5-g]quinoxalin-6-yl)phenyl]methyl]piperidin-4-yl]-1H-benzimidazol-2-one, and a pharmaceutically-acceptable salt of any one of the foregoing.

71. The method of claim 70, wherein the cancer is selected from acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilms tumor.

72. The method of claim 70, wherein Compound 1 and the AKT inhibitor are administered sequentially.

73. The method of claim 70, wherein Compound 1 is administered before the AKT inhibitor.

74. The method of claim 70, wherein the AKT inhibitor is administered before Compound 1.

75. The method of claim 70, wherein Compound 1 and the AKT inhibitor are administered concurrently.

76. The method of claim 70, wherein the cancer is ovarian cancer.

77. The method of claim 70, wherein the cancer is endometrial cancer.

78. The method of claim 70, wherein the cancer is lung cancer.

79. The method of claim 70, wherein the cancer is breast cancer.

80. The method of claim 70, wherein the cancer is acute myeloid leukemia.

81. The method of claim 70, wherein the cancer expresses a mutant p53 protein.

82. The method of claim 81, wherein the mutant p53 protein has a mutation at amino acid 220.

83. The method of claim 82, wherein the mutant p53 protein is p53 Y220C.


SEQUENCE LISTING (1 sequence)

SEQ ID NO: 1 - protein, 217 aa, Homo sapiens
SSSVPSQKTYQGSYGFRLGFLHSGTAKSVTCTYSPALNKMFCQLAKTCPVQLWVDSTPPPGTRVRAMAIYKQSQHMTEWRRCPHHERCSDSDGLAPPQHLIRVEGNLRVEYLDDRNTFRHSWVPCEPPEVGSDCTTIHYNYMCNSSCMGGMNRRPILTIITLEDSSGNLLGRNSFEVHVCACPGRDRRTEEENLRKKGEPHHELPPGSTKRALSNNT